(19) United States
(12) Patent Application Publication
(10) Pub. No.: US 2005/0260727 A1
(43) Pub. Date: Nov. 24, 2005

(54) Crystal structure of the mitochondrial protoporphyrinogen IX oxidase

(76) Inventors: Jorg Freigang, Langenfeld (DE); Michael Koch, Munchen (DE); Constanze Breithaupt, Munchen (DE); Reiner Kiefersauer, Neuried (DE); Albrecht Messerschmidt, Munchen (DE); Robert Huber, Germering (DE)

Correspondence Address: BAYER CROPSCIENCE LP;Patent Department, 100 BAYER ROAD, PITTSBURGH, PA, 15205-9741, US

(21) Appl. No.: 11/078,991

(22) Filed: Mar. 11, 2005

(30) Foreign Application Priority Data
Mar. 15, 2004 (DE) 102004012730.1
May 24, 2004 (DE) 102004025412.5

(51) Int. Cl. C12N 9/02; G06F 19/00; G01N 33/48; G01N 33/50

(52) U.S. Cl. 435/189; 702/19

(57) ABSTRACT

The present invention relates to crystals of a protoporphyrinogen IX oxidase, to processes for preparing crystals of a protoporphyrinogen IX oxidase, to processes for determining the structure of protoporphyrinogen IX oxidases alone or complexed with a ligand, and to processes for identifying ligands of the mitochondrial protoporphyrinogen IX oxidase, which may be used as herbicides. The present invention also relates to the use of the crystal structure described here of the Nicotiana tabacum mitochondrial protoporphyrinogen IX oxidase for generating protein models of related enzymes and to the identification of inhibitors of related proteins.

The present invention relates to crystals of a protoporphyrinogen IX oxidase, to processes for preparing crystals of a protoporphyrinogen IX oxidase, to processes for determining the structure of protoporphyrinogen IX oxidases alone or complexed with a ligand, and to processes for identifying ligands of the mitochondrial protoporphyrinogen IX oxidase, which may be used as herbicides. The present invention also relates to the use of the crystal structure described here of the Nicotiana tabacum mitochondrial protoporphyrinogen IX oxidase for generating protein models of related enzymes and to the identification of inhibitors of related proteins.

The enzymic activity of protoporphyrinogen IX oxidase (also referred to as “PPO” hereinbelow, for short) consists of catalyzing the oxidation Of protoporphyrinogen IX (formula I) to protoporphyrin IX (formula II) by molecular oxygen:

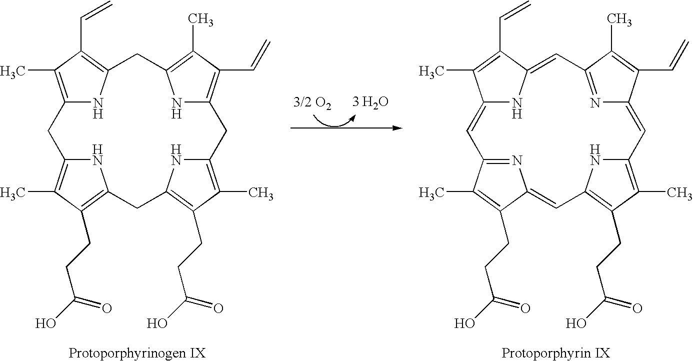

The protein is a representative of a highly conserved enzyme family for which membrane association is presumably achieved by way of a conserved, hydrophobic region in the protein (Arnould et al., 1999). In plants, there are two isoforms of the enzyme, namely the plastidic and the mitochondrial protoporphyrinogen IX oxidase (Lermontova et al., 1997). While the plastidic isoform is located in thylakoid membranes and the inner and outer plasma membranes, the mitochondrial form is located in the inner membrane of mitochondria.

Protoporphyrinogen IX oxidase is known to be an important target of peroxidizing herbicides, such as, for example, acifluorfen. Inhibition of the enzyme results in an accumulation of protoporphyrin (which forms by spontaneous reaction of protoporphyrinogen Ix. The decoupling of protoporphyrin formation and subsequent introduction of an iron ion, catalyzed by the protein ferrochelatase results in a loss of feedback control of haem biosynthesis by the final product haem. The photosensitive protophorphyrin IX accumulated thereby generates singulet oxygen which leads to cell death by way of peroxidation of lipids. The observed bleaching of photosynthetically active plant parts can be attributed to this effect (Lermontova et al., 1997). Protoporphyrinogen IX oxidase is therefore an enzyme particularly suitable for the search for further, improved herbicidal active compounds. In this context, in particular, the spatial structure of this protein is also of particular interest, which structure could make it possible to identify inhibitors of the enzyme and thus potential herbicides without complicated biochemical processes.

### DESCRIPTION OF THE FIGURES AND OF THE SEQUENCE LISTING

FIG. 1: FIG. 1 depicts the structure of the PPO homodimer with the bound inhibitor 4-bromo-3-(5′-carboxy-4′-chloro-2′-fluorophenyl)-1-methyl-5-trifluoromethyl-pyrazole (labelled INH), co-factor FAD and unspecifically bound Triton X-100 detergent molecule.

FIG. 2: FIG. 2 depicts a schematic representation of the PPO ligand-binding pocket with the bound inhibitor 4-bromo-3-(5′-carboxy-4′-chloro-2′-fluorophenyl)-1-methyl-5-trifluoromethylpyrazole.

SEQ ID NO: 1: SEQ ID NO: 1 (PPO) depicts the Nicotiana tabacum PPO amino acid sequence with mutations 1M and L224Q:

It was the object of the present invention to provide the spatial structure of the crystalline form of mitochondrial protoporphyrinogen IX oxidase and, in particular, the spatial structure of the PPO ligand-binding pocket, in order to make it possible for the said structure to be used in the search for novel herbicidal active compounds.

The object was achieved by providing a crystal of the PPO complexed with the inhibitor 4-bromo-3-(5′-carboxy-4′-chloro-2′-fluorophenyl)-1-methyl-5-trifluoromethylpyrazole, the structural coordinates of this crystal and processes for identifying ligands of the PPO or inhibitors of the enzymic activity of this enzyme.

The present invention thus relates to the crystal of a PPO, in particular a plant PPO. The crystalline PPO according to the invention is preferably that from Nicotiana tabacum. Particular preference is given to the PPO according to the invention comprising the amino acid sequence according to SEQ ID NO: 1.

The crystal of a PPO according to the invention is preferably a PPO with the bound inhibitor 4-bromo-3-(5′-carboxy-4′-chloro-2′-fluorophenyl)-1-methyl-5-trifluoromethylpyrazole. The crystal according to the invention of a PPO with the bound inhibitor 4-bromo-3-(5′-carboxy-4′-chloro-2′-fluorophenyl)-1-methyl-5-trifluoromethylpyrazole has preferably the structural coordinates defined in Table 2.

The invention relates in particular to a crystal having the space group C222, and the cell constants a=119.1 Å, b=147.3 Å, c=127.0 Å (see also Table 1). Such a structure also comprises a crystal having the structural coordinates according to Table 2.

The invention also relates to a process for preparing a crystal according to the invention, in which a selenomethionine-substituted protein is used. The invention further relates to a process for preparing a crystal according to the invention, in which a PPO is recombinantly produced in a bacterial host (e.g. E. coli) in the presence of selenomethionine in the medium, the selenomethionine-substituted protein is obtained from the bacterial cells and purified and crystals of this protein are prepared. The crystals may be prepared by any common method, for example in a hanging drop.

The invention therefore also relates to a process for preparing a crystal according to the invention, in which the crystal is generated by vapour diffusion in a sitting drop, the crystallization solution comprising 10% polyethylene glycol 1000, 0.1 M sodium citrate/HCl, pH 4.0 and 0.2 M sodium chloride.

The three-dimensional structure was resolved with the aid of protein crystals accessible to X-ray structural analysis by means of anomalous dispersion at a single wavelength and fully refined at 2.9 Å resolution. The present invention thus also relates to the three-dimensional structure of the complex of PPO and bound inhibitor 4-bromo-3-(5′-carboxy-4′-chloro-2′-fluorophenyl)-1-methyl-5-trifluoromethylpyrazole, determinable on the basis of these structural coordinates.

The present invention therefore relates in particular to a crystal of a PPO as described above, which comprises a ligand-binding pocket which is defined by the following amino acids at a distance of 4.5 Å from the bound ligand: Arg⁹⁸, Ala¹⁷⁴, Gly¹⁷⁵, Thr¹⁷⁶, Cys¹⁷⁷, Gly¹⁷⁸, Leu³³⁴, Phe³⁵³, Gly³⁵⁴, Val³⁵⁵, Leu ³⁵⁶, Leu³⁶⁹ Gly³⁷⁰, Thr³⁷, Leu³⁷² Phe³⁹², Phe⁴³⁹. The sequence and positional information refers to the amino acids according to SEQ ID NO: 1.

The present invention furthermore relates to the crystal of a PPO-ligand complex which comprises a ligand-binding pocket which is defined by the above-described amino acids and in which one or more of these amino acids have been mutated. These mutations are preferably conservative mutations in which a replacement with an amino acid with similar physical properties takes place.

The coordinates of Table 2 represent the coordinates of the atoms in Angstrom. The coordinates are a relative set of data of positions which define a structure in three dimensions. Thus it is possible for a completely different set of data of coordinates, which is based on a different origin and/or axes, to represent nevertheless an equal or identical structure. If the relative positions of the atoms of the structure are changed in a way that, with superposition with the corresponding coordinates according to Table 2, the square root of the mean square deviation of the atoms of the peptide backbone of the protein is less than 1.5 Å, preferably less than 1.0 Å, particularly preferably less than 0.5 Å, then usually a structure is likewise obtained which corresponds essentially to a structure according to Table 2, with respect to its structural characteristics and to its usability for structure-based identification of ligands of the protein. It should also be noted that changing the number and/or positions of the water molecules and/or substrate molecules of Table 2 does not fundamentally impair the usability of the structural coordinates according to Table 2.

It is therefore considered as being encompassed by the scope of the present invention, if the structural coordinates shown in Table 2 are shifted towards a different origin and/or different axes, or if the relative positions of the atoms of the structure are changed in a way that the square root of the mean square deviation of the atoms of the peptide backbone of the protein is, in comparison with the coordinates according to Table 2, less than 1.5 Å, preferably less than 1.0 Å, particularly preferably less than 0.5 Å, or if the number and/or positions of the water molecules and/or the substrate molecules are changed.

The structure of the PPO complex may be changed by binding of a ligand or inhibitor to the PPO complex. These changes are often restricted to side-chain conformations, but it is also possible for entire groups of amino acids to move with their peptide backbone. In addition, typical conformational changes occur in enzymes with unoccupied ligand-binding pocket or in enzymes whose ligand-binding pocket is occupied by inhibitors. Such changes are likewise encompassed by the present invention.

The skilled worker knows that various of the processes according to the invention described herein often need only parts of rather than the whole set of data according to Table 2. For example, it is possible, as will be described hereinbelow, to use selected coordinates of the PPO crystal for modelling a chemical compound based on the structural data of a crystal according to the invention. Frequently, only 5, preferably 10, particularly preferably 50 and very particularly preferably 100, atoms of the PPO structure suffice in order to model a chemical compound capable of interacting with the PPO. Accordingly, it is also possible for the other processes described herein such as, for example, the homology-based modelling and the homology-based structure elucidation, to use all or only parts of the structural coordinates according to Table 2.

The above-described aspects of the invention represent, individually or combined with one another, preferred embodiments of the invention.

In addition to the three-dimensional structure described herein of a crystal of a PPO, which structure is already immediately highly important for the search for ligands and thus also for inhibitors of the enzyme, the present invention also relates to the use of the said structure in determining the structures of proteins homologous to the present PPO. Thus, structural prediction currently works satisfactorily, i.e. without any extraordinary complication, if the structure of a homologous protein, i.e. a protein of the same origin, is known. Then it is possible to assume that a similar 3D structure is present and to use the known structure as a template in order to generate a model for the 3D structure of the other protein by means of homology-based modelling (“homology modelling”). Thus, the three-dimensional PPO structure according to the invention also makes it possible to predict the three-dimensional structure of a PPO from organisms other than Nicotiana tabacum by modelling. Such protein models may be used in the same way as the three-dimensional structure resolved here. It is then also possible, by comparing the differences in the amino acid sequences, to predict differences in the ligand-binding pockets of the PPOs of various organisms. This is useful if specific ligands for specific organisms are searched for or if, in the reverse case, precisely unspecific ligands with broad action are searched for. In addition, the three-dimensional structure of the invention may also serve to generate protein models of other sequence-related enzymes.

If thus X-ray structural data for a PPO, for example from a different organism, for a complex of a PPO and a ligand are available, the structural data according to Table 2, provided here, may be used in order to interpret the obtained data in a simple manner and to produce a structure. For this purpose, processes are used which are familiar to the skilled worker. One process which may be used for this purpose is referred to as molecular replacement. Owing to this method, the new structure may be determined more rapidly and efficiently than when trying to produce this structure ab initio. Examples of suitable computer programs which are commonly used, are CNX (Brunger et al., 1998) and AMORE (Navaza, 1994).

In this context, the degree of homology of two protein structures correlates well with the degree of sequence identity. Usually, the method of molecular replacement may be used already with 40% identity between the sequence of the template and the sequence of the structure to be resolved. The term “homology” or “identity” is to be understood as meaning the number of amino acids corresponding to other proteins (identity), expressed in percentage. The identity is preferably determined by comparing a given sequence to other proteins with the aid of computer programs. If sequences which are compared to one another have different lengths, the identity is to be determined so as for the number of amino acids shared between the shorter sequence and the longer sequence to determine the percentage identity. The identity may be determined in a standard manner by means of known and publicly available computer programs such as, for example, ClustalW (Thompson et al., Nucleic Acids Research 22 (1994), 4673-4680). ClustalW, for example, is made publicly available by Julie Thompson (Thompson@EMBL-Heidelberg.DE) and Toby Gibson (Gibson@EMBL-Heidelberg.DE), European Molecular Biology Laboratory, Meyer-hofstrasse 1, D 69117 Heidelberg, Germany. ClustalW may likewise be downloaded from various Internet sites, inter alia at IGBMC (Institut de Génétique et de Biologie Moléculaire et Cellulaire, B. P.163, 67404 Ilikirch Cedex, France; ftp://ftp-igbmc.u-strasbg.fr/pub/) and at EBI (ftp://ftp.ebi.ac.ukipub/software/) and at all mirrored Internet sites of the EBI (European Bioinformatics Institute, Wellcome Trust Genome Campus, Hinxton, Cambridge CB 10 1SD, UK). If the ClustalW computer program, Version 1.8, is used, in order to determine the identity between, for example, a given reference protein and other proteins, the following parameters are to be set: KTUPLE=1, TOPDIAG=5, WINDOW=5, PAIRGAP=3, GAPOPEN=10, GAPEXTEND=0.05, GAPDIST=8, MAXDIV=40, MATRIX=GONNET, ENDGAPS(OFF), NOPGAP, NOHGAP. One possibility of finding similar sequences is carrying out sequence database searches. Here, one or more sequences are provided in a “query”. This query sequence is then compared to sequences contained in the selected databases by means of statistical computer programs. Such database queries (“blast searches”) are known to the skilled worker and may be carried out with various providers. If such a database query is carried out, for example, with the NCBI (National Center for Biotechnology Information, http://www.ncbi.nlm.nih.gov/), the standard settings given for the particular comparative query should be used. For protein sequence comparisons (“blastp”), these are the following settings: Limit entrez=not activated; Filter=low complexity activated; Expect value=10; word size=3; Matrix=BLOSUM62; Gap costs: Existence=11, Extension=1. The result of such a query lists, besides other parameters, also the proportion of identity between the query sequence and the similar sequences found in the databases. A protein according to the invention is therefore, in connection with the present invention, to be understood as meaning those proteins which, when at least one of the above-described methods for determining identity is used, have an identity of at least 40%, preferably of at least 50%, particularly preferably of at least 60%, further preferably of at least 70%, and in particular of at least 80%.

The present invention therefore also relates to a process for determining the structure of a protein having the enzymic activity of a PPO, which has preferably an identity of at least 40%, preferably of 50%, particularly preferably of 60%, and very particularly preferably of 70%, to the sequence according to SEQ ID NO: 1, which process comprises the following steps:
(a) aligning one of the amino acid sequences of a PPO, having an identity of at least 40% with SEQ ID NO: 1, with the amino acid sequence according to SEQ ID NO: 1,
(b) modelling the structure of the corresponding homologous regions on the basis of the structural data according to Table 2 for these regions, and
(c) determining the conformation of the PPO, with the conformation essentially retaining the structure of the corresponding homologous regions.

The term “homologous region(s)” refers to amino acid residues of two sequences, which are identical or similar (e.g. aliphatic, aromatic, polar, negatively charged, positively charged, etc.). Such residues are frequently referred to as “invariant” or “conserved”.

Preferably, any or all of steps (a) to (c) are carried out by means of computer-assisted modelling. This homology modelling is a process known to the skilled worker (see, for example, Greer, 1985 and Blundell et al., 1988).

The structures of amino acids located in non-conserved regions may be arranged manually by using the usual peptide geometry or simulation techniques, such as, for example, molecular dynamics computer simulation. The final step of the process is the improvement (refinement) of the overall structure by molecular dynamics and/or energy minimization.

The present invention also comprises mutants, with this term also referring to polypeptides which are obtained by replacing at least one amino acid of the PPO according to SEQ ID NO: 1 with another amino acid and/or by insertion and/or deletion of one amino acid and which still have the essentially same three-dimensional structure as the PPO according to Table 2. An essentially same structure here refers to a set of structure coordinates for which the square root of the mean square deviation is less than or equal to 2.0 Å when the structural coordinates are superimposed by the structural coordinates according to Table 2 and when this superposition contains at least 50%, preferably 70% and particularly preferably all of the C_{α} atoms of the PPO of the known structure.

Using the PPO crystal structure according to the invention enables databases which contain the structures of a large number of compounds which are interesting as ligands of a PPO or as inhibitors of the enzymic activity of a PPO (“candidate molecules”) to be screened with the aid of established automated computer processes (virtual screening). These candidate molecules are in general small organic (low-molecular-weight) molecules which are suitable for use as active compounds, for example in crop protection. Such active compounds should be accessible to chemical synthesis. It is possible to use, for example, algorithms such as FLEXX (Rarey et al., 1996) or GOLD (Jones et al., 1997) for virtual screening. This procedure enables compounds to be identified whose three-dimensional structure enables them to get into the binding pocket and to bind there, for example by forming hydrogen bonds, by hydrophobic interaction, by charge interactions, by van-der-Waals interactions or by dipole interactions. The compounds identified in this way may be synthesized and then be used as herbicides, for example.

The present invention therefore also relates to a process for identifying ligands of the PPO or inhibitors of its enzymic activity, which process comprises the following steps:
(a) producing a three-dimensional structure of a PPO,
(b) producing the three-dimensional structure of a candidate molecule, and
(c) fitting the three-dimensional structure of the candidate molecule into the three-dimensional structure of the PPO.

Preference is given to using the data of Table 2 for producing the three-dimensional structure of a PPO. However, it is also possible to use in the same way the structural coordinates of other PPOs homologous to the present PPO according to SEQ ID NO: 1, which have been determined using a process as described above, based on the structural coordinates according to Table 2.

As an alternative to this, it is also possible to use only the coordinates of the atoms of the PPO according to Table 2, which are located in spatial proximity of the binding region of the putative ligand or inhibitor. These may be utilized in order to model a ligand-binding pocket in which the putative ligand or inhibitor can bind. These coordinates are thus used for defining a space which may then be utilized “in silico” for screening candidate molecules with the aid of suitable computer-based processes, as indicated above.

The present invention therefore also relates to a process for identifying ligands of the PPO or inhibitors of its enzymic activity, which process comprises the following steps:
(a) producing the coordinates of at least two atoms of a PPO (“selected coordinates”),
(b) producing the three-dimensional structure of a candidate molecule, and
(c) fitting the three-dimensional structure of the candidate molecule to the selected coordinates of the PPO.

Preference is given to using the data of Table 2 for producing the selected coordinates of a PPO. However, it is also possible to use in the same way the coordinates of other PPOs homologous to the present PPO according to SEQ ID NO: 1, which have been determined by a process as described above, based on the structural coordinates according to Table 2.

Usually, it will be advantageous if the model of the ligand-binding pocket is based on a sufficient number of atoms of a PPO. Preference is therefore given to using the coordinates of atoms of the residues Arg⁹⁸, Phe³⁹², Leu³⁵⁶ and Leu³⁷². Particular preference is given to using the coordinates of atoms of the residues Arg⁹⁸, Ala¹⁷⁴, Gly¹⁷⁵, Thr¹⁷⁶, Cys¹⁷⁷, Gly¹⁷⁸, Leu³³⁴, Phe³⁵³, Gly³⁵⁴, Val³⁵⁵, Leu 6, Leu³⁶⁹, Gly³⁷⁰, Thr³⁷¹, Leu³⁷², Phe³⁹², Phe⁴³⁹ according to SEQ ID NO: 1.

The term “fitting” describes the computer-assisted or partially computer-assisted determination of the interaction between at least one atom of the candidate molecule and at least one atom of the PPO and of calculating the stability of this interaction. Various processes known to the skilled worker are available for this fitting.

Another application of the three-dimensional structure of the PPO complex according to the invention is the generation of new ligands. To this end, structural formulae for new ligands which can enter the binding pocket and bind there, for example by forming hydrogen bonds, by hydrophobic interaction, by charge interactions, by van-der-Waals interactions or by dipole interactions, are generated using the structure and with the aid of established de novo design programs on a computer. Examples of possible de novo design programs are LUDI (Böhm, 1992), LEGEND (Nishibata & Itai, 1991) and GROW (Moon & Howe, 1991). Compounds generated in such a way may be synthesized and then likewise be used as herbicides, for example.

Ligands or potential inhibitors which either have been found in an in silico screening process as described above or have been generated and synthesized de novo as described above may be checked for their herbicidal activity in a subsequent step.

The processes according to the invention therefore comprise, where appropriate, also the following further steps:
(a) synthesis of the identified ligands or potential inhibitors,
(b) contacting these ligands or potential inhibitors with a PPO, and
(c) determining the enzymic activity of the PPO.

Alternatively, the ligands or potential inhibitors may also be contacted directly, where appropriate in a suitable formulation, with a plant or plant cells and the growth-inhibiting or killing action of the compounds on this plant may be determined.

Instead of or in addition to the said in vitro or in vivo processes for checking the identified compounds, the abovementioned processes may also comprise the following further steps:
(a) synthesis of the identified ligands or potential inhibitors,
(b) formation of a complex of a PPO and the ligand or potential inhibitor, and
(c) analysis of the complex by means of X-ray structural analysis to determine the ability of the ligand or potential inhibitor to interact with the PPO.

In this way it is possible to obtain detailed information on binding of the ligand and potential inhibitor. This information may also be utilized in order to adjust the structure of the ligand, for example in order to improve its effectiveness and/or specificity, for example for the PPO of a particular organism. The above-described steps of selecting a ligand by screening or its de novo generation, of testing in a biochemical in vitro or in an in vivo assay, and the analysis of the complex by means of determining the structure may also be repeated several times in order to gradually improve the characteristics of the ligand or inhibitor.

In this context, it is possible to determine the structure of the complex of a PPO and an identified ligand or inhibitor by means of the abovementioned process based on the structural coordinates according to Table 2 for determining the structure of a homologous PPO or of a new complex of a PPO according to SEQ ID NO:1 and a new ligand in an efficient manner. This may involve introducing a new ligand into PPO crystals already present by “soaking” in a manner known to the skilled worker. However, PPO and ligand may also be co-crystallized.

Depending on their particular physical and/or chemical properties, the identified active compounds may be transferred to the usual formulations such as solutions, emulsions, suspensions, powders, foams, pastes, granules, aerosols and microencapsulations in polymeric substances and in coating compositions for seeds, and ULV cool and warm fogging formulations.

These formulations are produced in a known manner, for example by mixing the active compounds with extenders, that is liquid solvents, liquefied gases under pressure, and/or solid carriers, optionally with the use of surfactants, that is emulsifiers and/or dispersants, and/or foam formers. If the extender used is water, it is also possible to employ, for example, organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide or dimethyl sulphoxide, or else water. Liquefied gaseous extenders or carriers are to be understood as meaning liquids which are gaseous at standard temperature and under atmospheric pressure, for example aerosol propellants such as halogenated hydrocarbons, or else butane, propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as finely divided silica, alumina and silicates. Suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks. Suitable emulsifiers and/or foam formers are: for example nonionogenic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates. Suitable dispersants are: for example lignosulphite waste liquors and methylcellulose.

Tackifiers such as carboxymethylcellulose, natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95 percent by weight of active compound, preferably between 0.5 and 90%.

The active compounds according to the invention can, as such or in their formulations, also be used in a mixture with known fungicides, bactericides, acaricides, nematicides or insecticides, to broaden, for example, the activity spectrum or to prevent development of resistance. In many cases, synergistic effects are obtained, i.e. the activity of the mixture is greater than the activity of the individual components.

When using the compounds according to the invention as fungicides, the application rates can be varied within a relatively wide range depending on the application.

According to the invention, it is possible to treat all plants and parts of plants. Plants are to be understood here as meaning all plants and plant populations, such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including plant cultivars which can or cannot be protected by plant breeders' certificates. Parts of plants are to be understood as meaning all above-ground and below-ground parts and organs of plants, such as shoot, leaf, flower and root, examples which may be mentioned being leaves, needles, stems, trunks, flowers, fruit-bodies, fruits and seeds and also roots, tubers and rhizomes. Parts of plants also include harvested material and vegetative and generative propagation material, for example seedlings, tubers, rhizomes, cuttings and seeds.

The treatment of the plants and parts of plants according to the invention with the active compounds is carried out directly or by action on their environment, habitat or storage area according to customary treatment methods, for example by dipping, spraying, evaporating, atomizing, broadcasting, brushing-on and, in the case of propagation material, in particular in the case of seeds, furthermore by one- or multilayer coating.

The following examples illustrate various aspects of the present invention and are not to be construed as limiting.

### EXAMPLES

### Example 1

Protein Preparation and Crystallization

During proteolytic cleavage of the fusion protein used, a further cleavage within the PPO was observed, in addition to the desired removal of the N-terminal part. In order to suppress this undesired secondary reaction, lysine 224 was mutated to glutamine 224. Nicotiana tabacum PPO was expressed in the form of fusion proteins with thioredoxin and a hexahistidine part with the aid of the expression vector pET32a. The bacterial strain used for this purpose was B834(DE3). The cells were cultured in minimal medium which contained selenomethionine instead of methionine. After the cells had been disrupted, a three-stage purification protocol was used to isolate the PPO. For this purpose, the cell lysate was purified in a first step by column chromatography on an Ni-NTA gel. After the amino-terminal fusion part had been proteolytically removed, the protein was subsequently chromatographically purified via a gel filtration column (Sephacryl S-200) and then via an anion exchange column (ResourceQ). For crystallization, the protein was transferred to a buffer containing 5 mM Tris/HCl, pH 8.0, 0.1% Triton X-100 and 50 mM sodium chloride. The crystals were generated by the method of vapour diffusion in a hanging drop. The crystallization solution used contained 10% polyethylene glycol 1000, 0.1 M sodium citrate/HCl, pH 4.0 and 0.2 M sodium chloride. Crystals with dimensions of 150×100×100 μm³ formed within 2 days. The intensely yellow crystals belonged to the C222₁ space group and had axis lengths of a=119.1 Å, b=147.3 Å and c=127.0 Å.

### Example 2

Crystallographic Data Recording and Analysis

The crystals which initially diffracted only to a resolution of approx. 8 Å were transformed into a different crystal form with the aid of a Free-Mounting System (Proteros biostructures GmbH, Martinsried, Germany). X-ray diffraction data were recorded at a wavelength of 0.9790 Å at the ESRF (European Radiotion Synchrotron Facility) in Grenoble, France. The diffraction images were processed using the program XDS (Kabsch, 1993). Anomalous scattering centres were identified by the program SnB (Weeks & Miller, 1999). Heavy metal atom parameters were refined using the program SHARP (Fortell & Bricogne, 1997). The structural model was modelled to the resulting electron density using the program o (Jones et al., 1991). Iterative refinement of the model was carried out using the program CNS (Brünger et al., 1998) (cf. Table 1).

### Example 3

Architecture of the Inhibitor Complex of the PPO

The 503 amino acids of the PPO form a compact overall structure which comprises three domains: an FAD-binding domain, a substrate-binding domain with a topology similar to p-hydroxybenzoate hydrolase and a membrane-binding domain. The structural moieties involved in FAD binding exhibit substantial sequential and structural homologies to other flavoenzymes such as human monoamine oxidase B (Binda et al., 2002), with an rmsd (root mean square deviation) of 1.68 Å over 256 Ca atoms and a sequence identity of 15.1%, polyamine oxidase (Binda et al., 1999), with an rmsd of 1.71 Å over 190 Ca atoms and a sequence identity of 14.9% and D-amino acid oxidase (Mitsutani et al., 1996), with an rmsd of 1.98 Å over 148 Ca atoms and a sequence identity of 14.9%. The model implies that the eight helices, α4 to α11, which include the conserved sequence region from residue 150 to residue 213 represent the membrane anchor and are presumably monotopically embedded in the membrane. In this model, predominantly apolar residues are oriented towards the hydrophobic moiety of the membrane. Charged residues which are likewise located in the membrane-embedded surface region are involved in the formation of salt bridges or may reach the positively charged sugar phosphate region of the membrane. These residues are exclusively lysines or histidines. In the PPO crystal structure, the membrane-binding domains of in each case four protomers are in contact with one another, with two contacts covering in each case 800 Å³ of solvent-accessible surface. Two further contacts each covering 400 Å³ of surface exist perpendicularly thereto. The latter are presumably the contacts which build the biologically relevant dimer. This interaction could be stabilized in vivo additionally by phospholipids of the surrounding membrane. In the dimeric arrangement of two PPO protomers, the active sites are on the outside of the dimer, close to the membrane surface. The dimer contact area is formed by the helices α5 and α14, with substantial contributions from leucine residues which interact with the corresponding leucine residues in the second monomer. This results in a T-shaped dimer in which two membrane-binding domains form a hydrophobic surface area which is extraordinarily suitable for insertion into the membrane. This architecture is similar to the likewise monotopically membrane-bound prostaglandin H₂ synthase (Loll et al., 1995).

### Example 4

The Active Site and the Structural Basis of Catalysis

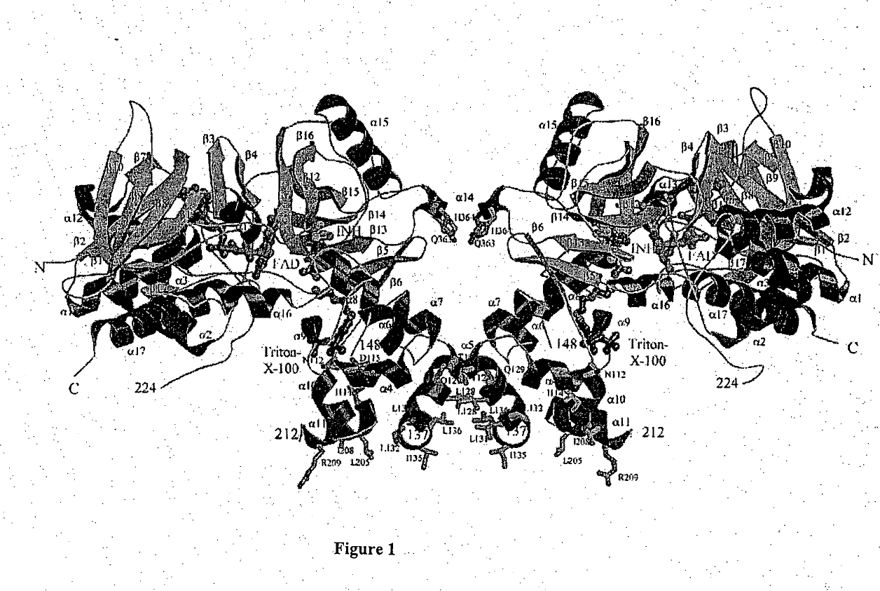

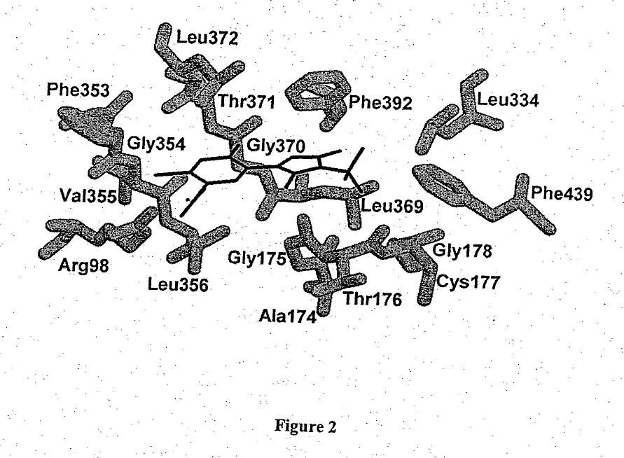

The active site of the PPO is located between the FAD- and the substrate-binding domain. The inhibitor binds to the active site in a tunnel-shaped inversion close to the FAD cofactor in a stretched conformation (FIGS. 1 and 2). The substrate-binding site beside the FAD is formed by a shallow indentation surrounded by a multiplicity of aliphatic and aromatic amino acids and Asn67 and Arg98. The crystal structure implies a model for substrate and product binding, in which the negatively charged propionyl groups are oriented in the direction of the solvent-exposed parts of the indentation at the active site. Arg98 which is highly conserved in all eukaryotic PPOs provides the countercharge to the carboxyl group of the inhibitor and probably also to the propionate group in the C ring of protoporphyrinogen IX. The pyrazole ring of the inhibitor serves as a model for binding of the A ring in protoporphyrinogen IX and is bound by aromatic interaction with the residue Phe392 which is conserved in all plant mitochondrial PPOs. A tyrosine replaces this residue in chloroplastic isoenzymes. The phenyl ring of the inhibitor presumably mimics binding of the protoporphyrinogen ring B and is trapped between the conserved residues Leu356 and Leu372. In the model described, the methylene bridge (C20) between the A and D rings of protoporphyrinogen IX is oriented in the direction of the reactive N5 atom of the FAD cofactor. This orientation implies that oxidation by FAD initially takes place at this site. Further oxidation should proceed from this position, since the narrow indentation at the active site should allow substrate binding only at this position and rule out rotation of the substrate. Hydrogen rearrangement via imine-enamine tautomerization enables all hydrids to be removed via the C20 position. The atom C10 between the A and C rings cannot get close enough to the N5 atom of FAD, since at this site the vinyl group is located on the C10 side of the molecule. FAD is reoxidized three times by molecular oxygen during the reaction, so as to give 3 hydrogen peroxide molecules. There are no residues in the immediate proximity of the substrate, which could act as a base. A plausible mechanism of deprotonation could comprise a reduction of molecular oxygen by a negatively charged FADH⁻, resulting in a peroxide anion which abstracts a proton from the solvent. The resulting hydroxide anion could then act as a general base.

### Example 5

In Vitro Assay for Determining the Enzymic Activity of a PPO in the Presence and Absence of a Ligand

The enzymic activity of the PPO was checked by firstly preparing a solution of freshly reduced protoporphyrinogen IX. For this purpose, 1.5 mg of commercially available protoporphyrin IX were admixed with 8 ml of 10 mM KOH and 2 ml of absolute ethanol. This solution was degassed using argon and added to 1.5 g of 5% strength sodium amalgam weighed under protective gas. The mixture was left at room temperature for 15 minutes and then titrated with 40% strength phosphoric acid to a pH of 7.0. 5 ml of the resulting solution were added through a sterile filter (pore size 0.45 μm, diameter 1 cm) to 25 ml of buffer solution containing 100 mM K₂HPO₄, 100 mM KH₂PO₄. 1 mM EDTA and 0.3% (v/v) TWEEN 80 at a pH of 7.2. The enzymic reaction was started by adding in each case 50 μl of enzymic solution (10 μg/ml in 5 mM Tris/HCl, 50 mM NaCl, pH 8.0) to 250 μl of the resulting reaction solution. The reaction progress was monitored by observing the fluorescence after 0, 5, 15 and 30 minutes, using a fluorescence spectrometer at an excitation wavelength of 410 nm and an emission wavelength of 635 nm. The effect of unspecific oxidation of the substrate by atmospheric oxygen was corrected by measuring a sample which did not contain any protein in parallel in the same manner. The activity resulting from this measurement was subtracted from the total activity in order to obtain the specific activity.

### Example 6

In Vivo Assay for Determining the Herbicidal Activity of a Ligand (Post-Emergence Assay)

| | |
|---|---|
| Solvent: | 5 parts by weight of acetone |
| Emulsifier: | 1 part by weight of alkylaryl polyglycol ether |

To produce a preparation of active compound, containing a candidate compound, 1 part by weight of active compound is mixed with the stated amount of solvent, the stated amount of emulsifier is added and the concentrate is diluted with water to the desired concentration.

Test plants which are from 5 to 15 cm in height are sprayed with the preparation of active compound in such a way that the amounts of active compound per unit area, desired in each case, are applied. The concentration of the spray liquor is chosen in such a way that the amounts of active compound desired in each case are applied in 1000 l of water per ha.

After three weeks, the degree of damage to the plants is rated in % damage in comparison to the development of the untreated control. The figures then denote: 0%=no effect (as untreated control), 100%=total destruction.

TABLE 1
Crystallographic data and refinement statistics (for details, see Example 2)

| Data recording: | |
|---|---|
| Resolution (Å; last shell) | 20-2.9 (3.0-2.9) |
| Space group | C222₁ |
| Cell constants (Å) | 119.1/147.3/127.0 |
| Mean redundancy | 3.8 (3.8) |
| R_{sym} (%; last shell)* | 7.9 (52.8) |
| Completion (%; last shell) | 100.0 (99.3) |
| Mean I/sigma (%; last shell) | 11.6 (2.8) |
| Refinement: | |
| Number of protein atoms | 6962 |
| Number of solvent positions | 143 |
| Number of ligand atoms | 192 |
| Mean square deviation of bond lengths | 0.008 |
| (Å) | |
| Mean square deviation of valence | 1.3 |
| angles (°) | |
| R_{cryst} (R_{free}) (%)§ | 22.7 (29.3) |

Values in parentheses correspond to the last shell of resolution
*R_{sym} (I) = Σₕₖₗ Σᵢ | I_{hkl,i} − < Iₕₖₗ > | / Σₕₖₗ Σᵢ | I_{hkl,i} | with <Iₕₖₗ > mean intensity of multiple I_{hkl,i} observations of symmetry-equivalent reflections.
Mean square deviations based on ideal values.
§R_{cryst} = Σₕₖₗ | F_{obs} − F_{calc} | / Σₕₖₗ |F_{obs} with F_{obs} and F_{calc} observed and calculated structural amplitudes, respectively. R_{free} is R_{cryst}, but calculated with those 10% of data not considered in the refinement.

TABLE 2
Structural coordinates of the PPO complex with 4-bromo-3-(5′-carboxy-4′-chloro-2′-
fluorophenyl)-1-methyl-5-trifluoromethylpyrazole

CRYST1 119.090 147.250 127.040 90.00 90.00 90.00 C 2 2 21

| | | | | | | | | | | | |
|---|---|---|---|---|---|---|---|---|---|---|---|
| ATOM | 1 | CB | ALA | A | 13 | −8.373 | 15.775 | 6.181 | 1.00 | 61.21 | A |
| ATOM | 2 | C | ALA | A | 13 | −7.743 | 14.967 | 8.483 | 1.00 | 62.97 | A |
| ATOM | 3 | O | ALA | A | 13 | −7.035 | 14.013 | 8.126 | 1.00 | 62.44 | A |
| ATOM | 4 | N | ALA | A | 13 | −6.757 | 17.096 | 7.497 | 1.00 | 61.00 | A |
| ATOM | 5 | CA | ALA | A | 13 | −7.955 | 16.206 | 7.583 | 1.00 | 61.88 | A |
| ATOM | 6 | N | LYS | A | 14 | −8.366 | 14.996 | 9.659 | 1.00 | 63.37 | A |
| ATOM | 7 | CA | LYS | A | 14 | −8.269 | 13.903 | 10.612 | 1.00 | 61.70 | A |
| ATOM | 8 | CB | LYS | A | 14 | −8.435 | 14.437 | 12.033 | 1.00 | 63.08 | A |
| ATOM | 9 | CG | LYS | A | 14 | −7.395 | 15.479 | 12.435 | 1.00 | 65.11 | A |
| ATOM | 10 | CD | LYS | A | 14 | −5.974 | 14.925 | 12.301 | 1.00 | 67.47 | A |
| ATOM | 11 | CE | LYS | A | 14 | −4.925 | 15.879 | 12.867 | 1.00 | 68.32 | A |
| ATOM | 12 | NZ | LYS | A | 14 | −5.009 | 16.019 | 14.352 | 1.00 | 69.10 | A |
| ATOM | 13 | C | LYS | A | 14 | −9.360 | 12.888 | 10.303 | 1.00 | 61.27 | A |
| ATOM | 14 | O | LYS | A | 14 | −10.426 | 13.247 | 9.780 | 1.00 | 60.70 | A |
| ATOM | 15 | N | ARG | A | 15 | −9.082 | 11.623 | 10.622 | 1.00 | 60.55 | A |
| ATOM | 16 | CA | ARG | A | 15 | −10.012 | 10.517 | 10.385 | 1.00 | 58.73 | A |
| ATOM | 17 | CB | ARG | A | 15 | −9.470 | 9.574 | 9.308 | 1.00 | 61.50 | A |
| ATOM | 18 | CG | ARG | A | 15 | −9.223 | 10.224 | 7.951 | 1.00 | 65.85 | A |
| ATOM | 19 | CD | ARG | A | 15 | −8.777 | 9.191 | 6.919 | 1.00 | 68.99 | A |
| ATOM | 20 | NE | ARG | A | 15 | −9.423 | 9.403 | 5.624 | 1.00 | 73.88 | A |
| ATOM | 21 | CZ | ARG | A | 15 | −10.724 | 9.226 | 5.395 | 1.00 | 75.89 | A |
| ATOM | 22 | NH1 | ARG | A | 15 | −11.531 | 8.826 | 6.376 | 1.00 | 76.97 | A |
| ATOM | 23 | NH2 | ARG | A | 15 | −11.222 | 9.458 | 4.185 | 1.00 | 76.71 | A |
| ATOM | 24 | C | ARG | A | 15 | −10.206 | 9.725 | 11.666 | 1.00 | 56.31 | A |
| ATOM | 25 | O | ARG | A | 15 | −9.248 | 9.452 | 12.383 | 1.00 | 54.96 | A |
| ATOM | 26 | N | VAL | A | 16 | −11.448 | 9.354 | 11.949 | 1.00 | 54.21 | A |
| ATOM | 27 | CA | VAL | A | 16 | −11.737 | 8.587 | 13.148 | 1.00 | 51.89 | A |
| ATOM | 28 | CB | VAL | A | 16 | −12.647 | 9.359 | 14.099 | 1.00 | 50.36 | A |
| ATOM | 29 | CG1 | VAL | A | 16 | −12.452 | 8.861 | 15.505 | 1.00 | 50.67 | A |
| ATOM | 30 | CG2 | VAL | A | 16 | −12.350 | 10.814 | 14.025 | 1.00 | 51.33 | A |
| ATOM | 31 | C | VAL | A | 16 | −12.406 | 7.248 | 12.842 | 1.00 | 50.71 | A |
| ATOM | 32 | O | VAL | A | 16 | −13.394 | 7.179 | 12.117 | 1.00 | 50.25 | A |
| ATOM | 33 | N | ALA | A | 17 | −11.844 | 6.186 | 13.402 | 1.00 | 49.53 | A |
| ATOM | 34 | CA | ALA | A | 17 | −12.380 | 4.852 | 13.230 | 1.00 | 48.36 | A |
| ATOM | 35 | CB | ALA | A | 17 | −11.285 | 3.836 | 13.401 | 1.00 | 47.70 | A |
| ATOM | 36 | C | ALA | A | 17 | −13.446 | 4.635 | 14.293 | 1.00 | 48.44 | A |
| ATOM | 37 | O | ALA | A | 17 | −13.286 | 5.024 | 15.450 | 1.00 | 50.40 | A |
| ATOM | 38 | N | VAL | A | 18 | −14.540 | 4.013 | 13.896 | 1.00 | 47.51 | A |
| ATOM | 39 | CA | VAL | A | 18 | −15.623 | 3.719 | 14.808 | 1.00 | 46.87 | A |
| ATOM | 40 | CB | VAL | A | 18 | −16.859 | 4.561 | 14.463 | 1.00 | 46.54 | A |
| ATOM | 41 | CG1 | VAL | A | 18 | −18.107 | 3.904 | 15.039 | 1.00 | 48.02 | A |
| ATOM | 42 | CG2 | VAL | A | 18 | −16.691 | 5.975 | 15.004 | 1.00 | 45.71 | A |
| ATOM | 43 | C | VAL | A | 18 | −15.965 | 2.238 | 14.665 | 1.00 | 47.15 | A |
| ATOM | 44 | O | VAL | A | 18 | −16.510 | 1.823 | 13.643 | 1.00 | 47.71 | A |
| ATOM | 45 | N | ILE | A | 19 | −15.638 | 1.433 | 15.673 | 1.00 | 46.44 | A |
| ATOM | 46 | CA | ILE | A | 19 | −15.950 | .006 | 15.605 | 1.00 | 45.53 | A |
| ATOM | 47 | CB | ILE | A | 19 | −14.912 | −.831 | 16.386 | 1.00 | 45.69 | A |
| ATOM | 48 | CG2 | ILE | A | 19 | −15.032 | −2.297 | 15.997 | 1.00 | 44.83 | A |
| ATOM | 49 | CG1 | ILE | A | 19 | −13.495 | −.373 | 16.041 | 1.00 | 45.83 | A |
| ATOM | 50 | CD1 | ILE | A | 19 | −12.415 | −1.150 | 16.783 | 1.00 | 45.66 | A |
| ATOM | 51 | C | ILE | A | 19 | −17.367 | −.284 | 16.151 | 1.00 | 44.58 | A |
| ATOM | 52 | O | ILE | A | 19 | −17.664 | −.071 | 17.327 | 1.00 | 40.96 | A |
| ATOM | 53 | N | GLY | A | 20 | −18.248 | −.754 | 15.273 | 1.00 | 45.16 | A |
| ATOM | 54 | CA | GLY | A | 20 | −19.612 | −1.055 | 15.689 | 1.00 | 44.22 | A |
| ATOM | 55 | C | GLY | A | 20 | −20.621 | −.076 | 15.132 | 1.00 | 43.01 | A |
| ATOM | 56 | O | GLY | A | 20 | −20.484 | 1.137 | 15.330 | 1.00 | 44.14 | A |
| ATOM | 57 | N | ALA | A | 21 | −21.626 | −.590 | 14.432 | 1.00 | 41.17 | A |
| ATOM | 58 | CA | ALA | A | 21 | −22.648 | .275 | 13.854 | 1.00 | 41.16 | A |
| ATOM | 59 | CB | ALA | A | 21 | −22.857 | −.073 | 12.409 | 1.00 | 43.21 | A |
| ATOM | 60 | C | ALA | A | 21 | −23.974 | .188 | 14.604 | 1.00 | 40.56 | A |
| ATOM | 61 | O | ALA | A | 21 | −25.035 | .139 | 13.997 | 1.00 | 39.01 | A |
| ATOM | 62 | N | GLY | A | 22 | −23.900 | .162 | 15.931 | 1.00 | 41.52 | A |
| ATOM | 63 | CA | GLY | A | 22 | −25.099 | .109 | 16.746 | 1.00 | 40.49 | A |
| ATOM | 64 | C | GLY | A | 22 | −25.527 | 1.532 | 16.992 | 1.00 | 40.28 | A |
| ATOM | 65 | O | GLY | A | 22 | −24.876 | 2.447 | 16.511 | 1.00 | 40.90 | A |
| ATOM | 66 | N | VAL | A | 23 | −26.606 | 1.744 | 17.726 | 1.00 | 40.76 | A |
| ATOM | 67 | CA | VAL | A | 23 | −27.042 | 3.112 | 17.972 | 1.00 | 41.78 | A |
| ATOM | 68 | CB | VAL | A | 23 | −28.247 | 3.184 | 18.940 | 1.00 | 40.35 | A |
| ATOM | 69 | CG1 | VAL | A | 23 | −27.794 | 2.921 | 20.368 | 1.00 | 39.78 | A |
| ATOM | 70 | CG2 | VAL | A | 23 | −28.888 | 4.537 | 18.840 | 1.00 | 38.08 | A |
| ATOM | 71 | C | VAL | A | 23 | −25.895 | 3.943 | 18.552 | 1.00 | 42.10 | A |
| ATOM | 72 | O | VAL | A | 23 | −25.756 | 5.113 | 18.234 | 1.00 | 44.27 | A |
| ATOM | 73 | N | SER | A | 24 | −25.064 | 3.338 | 19.387 | 1.00 | 41.46 | A |
| ATOM | 74 | CA | SER | A | 24 | −23.944 | 4.062 | 19.983 | 1.00 | 42.06 | A |
| ATOM | 75 | CB | SER | A | 24 | −23.281 | 3.211 | 21.047 | 1.00 | 42.40 | A |
| ATOM | 76 | OG | SER | A | 24 | −22.135 | 3.875 | 21.496 | 1.00 | 42.77 | A |
| ATOM | 77 | C | SER | A | 24 | −22.871 | 4.501 | 18.989 | 1.00 | 42.52 | A |
| ATOM | 78 | O | SER | A | 24 | −22.370 | 5.617 | 19.062 | 1.00 | 41.16 | A |
| ATOM | 79 | N | GLY | A | 25 | −22.503 | 3.603 | 18.079 | 1.00 | 44.41 | A |
| ATOM | 80 | CA | GLY | A | 25 | −21.495 | 3.914 | 17.081 | 1.00 | 44.77 | A |
| ATOM | 81 | C | GLY | A | 25 | −21.992 | 4.914 | 16.055 | 1.00 | 45.83 | A |
| ATOM | 82 | O | GLY | A | 25 | −21.225 | 5.773 | 15.605 | 1.00 | 45.61 | A |
| ATOM | 83 | N | LEU | A | 26 | −23.272 | 4.804 | 15.688 | 1.00 | 45.62 | A |
| ATOM | 84 | CA | LEU | A | 26 | −23.886 | 5.714 | 14.719 | 1.00 | 45.25 | A |
| ATOM | 85 | CB | LEU | A | 26 | −25.246 | 5.177 | 14.265 | 1.00 | 44.11 | A |
| ATOM | 86 | CG | LEU | A | 26 | −25.090 | 3.878 | 13.481 | 1.00 | 44.91 | A |
| ATOM | 87 | CD1 | LEU | A | 26 | −26.426 | 3.268 | 13.175 | 1.00 | 46.59 | A |
| ATOM | 88 | CD2 | LEU | A | 26 | −24.339 | 4.160 | 12.208 | 1.00 | 46.71 | A |
| ATOM | 89 | C | LEU | A | 26 | −24.039 | 7.100 | 15.326 | 1.00 | 44.72 | A |
| ATOM | 90 | O | LEU | A | 26 | −23.894 | 8.112 | 14.636 | 1.00 | 43.23 | A |
| ATOM | 91 | N | ALA | A | 27 | −24.319 | 7.133 | 16.624 | 1.00 | 44.01 | A |
| ATOM | 92 | CA | ALA | A | 27 | −24.475 | 8.384 | 17.342 | 1.00 | 45.02 | A |
| ATOM | 93 | CB | ALA | A | 27 | −24.866 | 8.117 | 18.789 | 1.00 | 45.77 | A |
| ATOM | 94 | C | ALA | A | 27 | −23.142 | 9.095 | 17.293 | 1.00 | 45.57 | A |
| ATOM | 95 | O | ALA | A | 27 | −23.067 | 10.268 | 16.933 | 1.00 | 45.38 | A |
| ATOM | 96 | N | ALA | A | 28 | −22.094 | 8.365 | 17.662 | 1.00 | 46.15 | A |
| ATOM | 97 | CA | ALA | A | 28 | −20.733 | 8.883 | 17.667 | 1.00 | 47.43 | A |
| ATOM | 98 | CB | ALA | A | 28 | −19.799 | 7.826 | 18.190 | 1.00 | 47.63 | A |
| ATOM | 99 | C | ALA | A | 28 | −20.287 | 9.332 | 16.272 | 1.00 | 48.35 | A |
| ATOM | 100 | O | ALA | A | 28 | −19.699 | 10.401 | 16.110 | 1.00 | 46.20 | A |
| ATOM | 101 | N | ALA | A | 29 | −20.566 | 8.513 | 15.265 | 1.00 | 50.47 | A |
| ATOM | 102 | CA | ALA | A | 29 | −20.200 | 8.852 | 13.891 | 1.00 | 53.79 | A |
| ATOM | 103 | CB | ALA | A | 29 | −20.521 | 7.694 | 12.968 | 1.00 | 51.88 | A |
| ATOM | 104 | C | ALA | A | 29 | −20.905 | 10.118 | 13.383 | 1.00 | 56.14 | A |
| ATOM | 105 | O | ALA | A | 29 | −20.318 | 10.904 | 12.631 | 1.00 | 56.59 | A |
| ATOM | 106 | N | TYR | A | 30 | −22.161 | 10.302 | 13.797 | 1.00 | 57.00 | A |
| ATOM | 107 | CA | TYR | A | 30 | −22.970 | 11.445 | 13.383 | 1.00 | 55.84 | A |
| ATOM | 108 | CB | TYR | A | 30 | −24.422 | 11.195 | 13.740 | 1.00 | 56.70 | A |
| ATOM | 109 | CG | TYR | A | 30 | −25.351 | 12.247 | 13.213 | 1.00 | 58.60 | A |
| ATOM | 110 | CD1 | TYR | A | 30 | −25.790 | 12.209 | 11.897 | 1.00 | 59.44 | A |
| ATOM | 111 | CE1 | TYR | A | 30 | −26.638 | 13.185 | 11.392 | 1.00 | 61.84 | A |
| ATOM | 112 | CD2 | TYR | A | 30 | −25.782 | 13.295 | 14.025 | 1.00 | 60.07 | A |
| ATOM | 113 | CE2 | TYR | A | 30 | −26.630 | 14.282 | 13.531 | 1.00 | 62.42 | A |
| ATOM | 114 | CZ | TYR | A | 30 | −27.057 | 14.216 | 12.210 | 1.00 | 62.76 | A |
| ATOM | 115 | OH | TYR | A | 30 | −27.930 | 15.155 | 11.713 | 1.00 | 63.54 | A |
| ATOM | 116 | C | TYR | A | 30 | −22.517 | 12.737 | 14.039 | 1.00 | 55.88 | A |
| ATOM | 117 | O | TYR | A | 30 | −22.343 | 13.762 | 13.387 | 1.00 | 53.57 | A |
| ATOM | 118 | N | LYS | A | 31 | −22.349 | 12.679 | 15.350 | 1.00 | 57.51 | A |
| ATOM | 119 | CA | LYS | A | 31 | −21.906 | 13.828 | 16.116 | 1.00 | 58.73 | A |
| ATOM | 120 | CB | LYS | A | 31 | −21.784 | 13.439 | 17.587 | 1.00 | 60.03 | A |
| ATOM | 121 | CG | LYS | A | 31 | −21.959 | 14.574 | 18.554 | 1.00 | 63.09 | A |
| ATOM | 122 | CD | LYS | A | 31 | −23.394 | 15.029 | 18.576 | 1.00 | 66.17 | A |
| ATOM | 123 | CE | LYS | A | 31 | −23.635 | 16.009 | 19.712 | 1.00 | 69.89 | A |
| ATOM | 124 | NZ | LYS | A | 31 | −25.094 | 16.190 | 19.989 | 1.00 | 72.01 | A |
| ATOM | 125 | C | LYS | A | 31 | −20.538 | 14.250 | 15.567 | 1.00 | 58.38 | A |
| ATOM | 126 | O | LYS | A | 31 | −20.162 | 15.418 | 15.632 | 1.00 | 59.61 | A |
| ATOM | 127 | N | LEU | A | 32 | −19.798 | 13.281 | 15.033 | 1.00 | 57.55 | A |
| ATOM | 128 | CA | LEU | A | 32 | −18.486 | 13.529 | 14.461 | 1.00 | 55.02 | A |
| ATOM | 129 | CB | LEU | A | 32 | −17.727 | 12.217 | 14.287 | 1.00 | 53.59 | A |
| ATOM | 130 | CG | LEU | A | 32 | −17.005 | 11.639 | 15.507 | 1.00 | 52.80 | A |
| ATOM | 131 | CD1 | LEU | A | 32 | −16.526 | 10.230 | 15.207 | 1.00 | 51.83 | A |
| ATOM | 132 | CD2 | LEU | A | 32 | −15.839 | 12.512 | 15.877 | 1.00 | 50.33 | A |
| ATOM | 133 | C | LEU | A | 32 | −18.586 | 14.231 | 13.120 | 1.00 | 55.47 | A |
| ATOM | 134 | O | LEU | A | 32 | −17.944 | 15.248 | 12.920 | 1.00 | 56.78 | A |
| ATOM | 135 | N | LYS | A | 33 | −19.382 | 13.697 | 12.198 | 1.00 | 56.17 | A |
| ATOM | 136 | CA | LYS | A | 33 | −19.525 | 14.311 | 10.875 | 1.00 | 57.17 | A |
| ATOM | 137 | CB | LYS | A | 33 | −20.506 | 13.527 | 10.008 | 1.00 | 56.42 | A |
| ATOM | 138 | CG | LYS | A | 33 | −19.980 | 12.201 | 9.519 | 1.00 | 59.68 | A |
| ATOM | 139 | CD | LYS | A | 33 | −20.724 | 11.730 | 8.271 | 1.00 | 60.78 | A |
| ATOM | 140 | CE | LYS | A | 33 | −20.348 | 12.573 | 7.052 | 1.00 | 62.78 | A |
| ATOM | 141 | NZ | LYS | A | 33 | −21.040 | 12.158 | 5.789 | 1.00 | 62.80 | A |
| ATOM | 142 | C | LYS | A | 33 | −19.970 | 15.775 | 10.896 | 1.00 | 58.41 | A |
| ATOM | 143 | O | LYS | A | 33 | −19.374 | 16.625 | 10.233 | 1.00 | 59.41 | A |
| ATOM | 144 | N | ILE | A | 34 | −21.013 | 16.076 | 11.659 | 1.00 | 58.65 | A |
| ATOM | 145 | CA | ILE | A | 34 | −21.513 | 17.432 | 11.705 | 1.00 | 56.49 | A |
| ATOM | 146 | CB | ILE | A | 34 | −22.851 | 17.509 | 12.474 | 1.00 | 56.26 | A |
| ATOM | 147 | CG2 | ILE | A | 34 | −23.785 | 16.417 | 11.983 | 1.00 | 55.57 | A |
| ATOM | 148 | CG1 | ILE | A | 34 | −22.622 | 17.354 | 13.971 | 1.00 | 56.30 | A |
| ATOM | 149 | CD1 | ILE | A | 34 | −23.884 | 17.515 | 14.783 | 1.00 | 56.70 | A |
| ATOM | 150 | C | ILE | A | 34 | −20.500 | 18.391 | 12.304 | 1.00 | 55.95 | A |
| ATOM | 151 | O | ILE | A | 34 | −20.803 | 19.564 | 12.528 | 1.00 | 57.52 | A |
| ATOM | 152 | N | HIS | A | 35 | −19.295 | 17.892 | 12.558 | 1.00 | 54.67 | A |
| ATOM | 153 | CA | HIS | A | 35 | −18.222 | 18.722 | 13.101 | 1.00 | 55.73 | A |
| ATOM | 154 | CB | HIS | A | 35 | −17.864 | 18.315 | 14.538 | 1.00 | 57.77 | A |
| ATOM | 155 | CG | HIS | A | 35 | −16.801 | 19.165 | 15.168 | 1.00 | 59.25 | A |
| ATOM | 156 | CD2 | HIS | A | 35 | −16.876 | 20.153 | 16.093 | 1.00 | 61.14 | A |
| ATOM | 157 | ND1 | HIS | A | 35 | −15.464 | 19.031 | 14.865 | 1.00 | 59.17 | A |
| ATOM | 158 | CE1 | HIS | A | 35 | −14.760 | 19.894 | 15.577 | 1.00 | 59.90 | A |
| ATOM | 159 | NE2 | HIS | A | 35 | −15.592 | 20.587 | 16.331 | 1.00 | 60.87 | A |
| ATOM | 160 | C | HIS | A | 35 | −16.999 | 18.612 | 12.205 | 1.00 | 54.99 | A |
| ATOM | 161 | O | HIS | A | 35 | −15.881 | 18.828 | 12.646 | 1.00 | 53.31 | A |
| ATOM | 162 | N | GLY | A | 36 | −17.234 | 18.263 | 10.940 | 1.00 | 54.49 | A |
| ATOM | 163 | CA | GLY | A | 36 | −16.159 | 18.168 | 9.968 | 1.00 | 55.64 | A |
| ATOM | 164 | C | GLY | A | 36 | −15.432 | 16.846 | 9.811 | 1.00 | 56.04 | A |
| ATOM | 165 | O | GLY | A | 36 | −15.339 | 16.304 | 8.712 | 1.00 | 55.40 | A |
| ATOM | 166 | N | LEU | A | 37 | −14.901 | 16.337 | 10.913 | 1.00 | 57.11 | A |
| ATOM | 167 | CA | LEU | A | 37 | −14.168 | 15.080 | 10.923 | 1.00 | 57.40 | A |
| ATOM | 168 | CB | LEU | A | 37 | −14.086 | 14.560 | 12.363 | 1.00 | 55.35 | A |
| ATOM | 169 | CG | LEU | A | 37 | −13.517 | 15.538 | 13.397 | 1.00 | 52.69 | A |
| ATOM | 170 | CD1 | LEU | A | 37 | −13.413 | 14.850 | 14.733 | 1.00 | 53.42 | A |
| ATOM | 171 | CD2 | LEU | A | 37 | −12.155 | 16.019 | 12.969 | 1.00 | 53.78 | A |
| ATOM | 172 | C | LEU | A | 37 | −14.727 | 13.988 | 9.987 | 1.00 | 57.88 | A |
| ATOM | 173 | O | LEU | A | 37 | −15.904 | 13.635 | 10.044 | 1.00 | 57.44 | A |
| ATOM | 174 | N | ASN | A | 38 | −13.858 | 13.480 | 9.117 | 1.00 | 58.43 | A |
| ATOM | 175 | CA | ASN | A | 38 | −14.191 | 12.420 | 8.175 | 1.00 | 60.22 | A |
| ATOM | 176 | CB | ASN | A | 38 | −13.100 | 12.310 | 7.110 | 1.00 | 65.16 | A |
| ATOM | 177 | CG | ASN | A | 38 | −12.765 | 13.650 | 6.464 | 1.00 | 68.12 | A |
| ATOM | 178 | OD1 | ASN | A | 38 | −13.566 | 14.205 | 5.700 | 1.00 | 69.61 | A |
| ATOM | 179 | ND2 | ASN | A | 38 | −11.576 | 14.177 | 6.770 | 1.00 | 67.83 | A |
| ATOM | 180 | C | ASN | A | 38 | −14.229 | 11.122 | 8.989 | 1.00 | 60.60 | A |
| ATOM | 181 | O | ASN | A | 38 | −13.304 | 10.832 | 9.755 | 1.00 | 60.93 | A |
| ATOM | 182 | N | VAL | A | 39 | −15.278 | 10.328 | 8.804 | 1.00 | 59.41 | A |
| ATOM | 183 | CA | VAL | A | 39 | −15.442 | 9.104 | 9.575 | 1.00 | 57.11 | A |
| ATOM | 184 | CB | VAL | A | 39 | −16.680 | 9.212 | 10.467 | 1.00 | 55.85 | A |
| ATOM | 185 | CG1 | VAL | A | 39 | −16.793 | 7.980 | 11.349 | 1.00 | 54.95 | A |
| ATOM | 186 | CG2 | VAL | A | 39 | −16.623 | 10.493 | 11.280 | 1.00 | 52.78 | A |
| ATOM | 187 | C | VAL | A | 39 | −15.597 | 7.830 | 8.768 | 1.00 | 57.35 | A |
| ATOM | 188 | O | VAL | A | 39 | −16.086 | 7.854 | 7.637 | 1.00 | 59.26 | A |
| ATOM | 189 | N | THR | A | 40 | −15.190 | 6.715 | 9.370 | 1.00 | 55.76 | A |
| ATOM | 190 | CA | THR | A | 40 | −15.313 | 5.391 | 8.759 | 1.00 | 54.44 | A |
| ATOM | 191 | CB | THR | A | 40 | −13.951 | 4.817 | 8.305 | 1.00 | 54.99 | A |
| ATOM | 192 | OG1 | THR | A | 40 | −13.442 | 5.584 | 7.212 | 1.00 | 57.09 | A |
| ATOM | 193 | CG2 | THR | A | 40 | −14.107 | 3.395 | 7.838 | 1.00 | 56.37 | A |
| ATOM | 194 | C | THR | A | 40 | −15.887 | 4.486 | 9.839 | 1.00 | 52.95 | A |
| ATOM | 195 | O | THR | A | 40 | −15.395 | 4.463 | 10.962 | 1.00 | 53.20 | A |
| ATOM | 196 | N | VAL | A | 41 | −16.928 | 3.742 | 9.502 | 1.00 | 50.87 | A |
| ATOM | 197 | CA | VAL | A | 41 | −17.561 | 2.864 | 10.467 | 1.00 | 49.92 | A |
| ATOM | 198 | CB | VAL | A | 41 | −19.034 | 3.243 | 10.646 | 1.00 | 49.76 | A |
| ATOM | 199 | CG1 | VAL | A | 41 | −19.718 | 2.236 | 11.569 | 1.00 | 50.56 | A |
| ATOM | 200 | CG2 | VAL | A | 41 | −19.139 | 4.656 | 11.187 | 1.00 | 47.17 | A |
| ATOM | 201 | C | VAL | A | 41 | −17.506 | 1.392 | 10.100 | 1.00 | 49.10 | A |
| ATOM | 202 | O | VAL | A | 41 | −18.093 | .970 | 9.111 | 1.00 | 49.82 | A |
| ATOM | 203 | N | PHE | A | 42 | −16.815 | .602 | 10.901 | 1.00 | 48.61 | A |
| ATOM | 204 | CA | PHE | A | 42 | −16.746 | −.822 | 10.622 | 1.00 | 49.60 | A |
| ATOM | 205 | CB | PHE | A | 42 | −15.415 | −1.402 | 11.094 | 1.00 | 50.00 | A |
| ATOM | 206 | CG | PHE | A | 42 | −14.227 | −.771 | 10.450 | 1.00 | 51.38 | A |
| ATOM | 207 | CD1 | PHE | A | 42 | −13.792 | .482 | 10.850 | 1.00 | 51.26 | A |
| ATOM | 208 | CD2 | PHE | A | 42 | −13.552 | −1.420 | 9.421 | 1.00 | 50.89 | A |
| ATOM | 209 | CE1 | PHE | A | 42 | −12.698 | 1.080 | 10.236 | 1.00 | 51.40 | A |
| ATOM | 210 | CE2 | PHE | A | 42 | −12.459 | −.830 | 8.805 | 1.00 | 50.99 | A |
| ATOM | 211 | CZ | PHE | A | 42 | −12.031 | .422 | 9.213 | 1.00 | 50.63 | A |
| ATOM | 212 | C | PHE | A | 42 | −17.875 | −1.566 | 11.307 | 1.00 | 49.54 | A |
| ATOM | 213 | O | PHE | A | 42 | −18.086 | −1.408 | 12.510 | 1.00 | 49.28 | A |
| ATOM | 214 | N | GLU | A | 43 | −18.591 | −2.378 | 10.538 | 1.00 | 48.52 | A |
| ATOM | 215 | CA | GLU | A | 43 | −19.678 | −3.176 | 11.074 | 1.00 | 50.28 | A |
| ATOM | 216 | CB | GLU | A | 43 | −21.018 | −2.685 | 10.541 | 1.00 | 51.16 | A |
| ATOM | 217 | CG | GLU | A | 43 | −22.147 | −3.633 | 10.884 | 1.00 | 53.05 | A |
| ATOM | 218 | CD | GLU | A | 43 | −22.207 | −3.924 | 12.374 | 1.00 | 55.99 | A |
| ATOM | 219 | OE1 | GLU | A | 43 | −22.614 | −5.047 | 12.735 | 1.00 | 59.06 | A |
| ATOM | 220 | OE2 | GLU | A | 43 | −21.857 | −3.036 | 13.188 | 1.00 | 57.48 | A |
| ATOM | 221 | C | GLU | A | 43 | −19.521 | −4.668 | 10.753 | 1.00 | 51.28 | A |
| ATOM | 222 | O | GLU | A | 43 | −19.536 | −5.072 | 9.596 | 1.00 | 51.43 | A |
| ATOM | 223 | N | ALA | A | 44 | −19.383 | −5.487 | 11.786 | 1.00 | 52.63 | A |
| ATOM | 224 | CA | ALA | A | 44 | −19.222 | −6.923 | 11.597 | 1.00 | 54.12 | A |
| ATOM | 225 | CB | ALA | A | 44 | −19.008 | −7.589 | 12.939 | 1.00 | 51.08 | A |
| ATOM | 226 | C | ALA | A | 44 | −20.397 | −7.582 | 10.860 | 1.00 | 55.69 | A |
| ATOM | 227 | O | ALA | A | 44 | −20.202 | −8.419 | 9.974 | 1.00 | 55.28 | A |
| ATOM | 228 | N | GLU | A | 45 | −21.613 | −7.192 | 11.221 | 1.00 | 58.29 | A |
| ATOM | 229 | CA | GLU | A | 45 | −22.815 | −7.751 | 10.615 | 1.00 | 60.56 | A |
| ATOM | 230 | CB | GLU | A | 45 | −23.996 | −7.496 | 11.548 | 1.00 | 63.70 | A |
| ATOM | 231 | CG | GLU | A | 45 | −24.761 | −8.745 | 11.957 | 1.00 | 71.83 | A |
| ATOM | 232 | CD | GLU | A | 45 | −23.909 | −9.774 | 12.697 | 1.00 | 74.38 | A |
| ATOM | 233 | OE1 | GLU | A | 45 | −24.489 | −10.555 | 13.485 | 1.00 | 74.22 | A |
| ATOM | 234 | OE2 | GLU | A | 45 | −22.674 | −9.815 | 12.484 | 1.00 | 77.18 | A |
| ATOM | 235 | C | GLU | A | 45 | −23.123 | −7.222 | 9.205 | 1.00 | 60.04 | A |
| ATOM | 236 | O | GLU | A | 45 | −22.491 | −6.271 | 8.726 | 1.00 | 59.53 | A |
| ATOM | 237 | N | GLY | A | 46 | −24.102 | −7.845 | 8.551 | 1.00 | 59.04 | A |
| ATOM | 238 | CA | GLY | A | 46 | −24.484 | −7.446 | 7.205 | 1.00 | 58.57 | A |
| ATOM | 239 | C | GLY | A | 46 | −25.400 | −6.239 | 7.155 | 1.00 | 57.59 | A |
| ATOM | 240 | O | GLY | A | 46 | −25.522 | −5.592 | 6.116 | 1.00 | 56.56 | A |
| ATOM | 241 | N | LYS | A | 47 | −26.060 | −5.949 | 8.273 | 1.00 | 56.96 | A |
| ATOM | 242 | CA | LYS | A | 47 | −26.941 | −4.798 | 8.359 | 1.00 | 57.21 | A |
| ATOM | 243 | CB | LYS | A | 47 | −28.373 | −5.243 | 8.670 | 1.00 | 60.87 | A |
| ATOM | 244 | CG | LYS | A | 47 | −29.077 | −5.870 | 7.447 | 1.00 | 65.89 | A |
| ATOM | 245 | CD | LYS | A | 47 | −30.372 | −6.639 | 7.782 | 1.00 | 67.08 | A |
| ATOM | 246 | CE | LYS | A | 47 | −30.939 | −7.350 | 6.527 | 1.00 | 66.82 | A |
| ATOM | 247 | NZ | LYS | A | 47 | −32.098 | −8.263 | 6.811 | 1.00 | 65.01 | A |
| ATOM | 248 | C | LYS | A | 47 | −26.417 | −3.839 | 9.413 | 1.00 | 55.92 | A |
| ATOM | 249 | O | LYS | A | 47 | −25.748 | −4.245 | 10.351 | 1.00 | 55.78 | A |
| ATOM | 250 | N | ALA | A | 48 | −26.709 | −2.559 | 9.230 | 1.00 | 56.66 | A |
| ATOM | 251 | CA | ALA | A | 48 | −26.267 | −1.509 | 10.132 | 1.00 | 56.84 | A |
| ATOM | 252 | CB | ALA | A | 48 | −26.441 | −.156 | 9.470 | 1.00 | 57.67 | A |
| ATOM | 253 | C | ALA | A | 48 | −26.968 | −1.509 | 11.485 | 1.00 | 58.62 | A |
| ATOM | 254 | O | ALA | A | 48 | −26.306 | −1.358 | 12.512 | 1.00 | 61.68 | A |
| ATOM | 255 | N | GLY | A | 49 | −28.287 | −1.661 | 11.519 | 1.00 | 57.07 | A |
| ATOM | 256 | CA | GLY | A | 49 | −28.949 | −1.652 | 12.815 | 1.00 | 60.16 | A |
| ATOM | 257 | C | GLY | A | 49 | −28.113 | −2.314 | 13.916 | 1.00 | 60.95 | A |
| ATOM | 258 | O | GLY | A | 49 | −27.383 | −3.281 | 13.650 | 1.00 | 63.17 | A |
| ATOM | 259 | N | GLY | A | 50 | −28.175 | −1.816 | 15.146 | 1.00 | 58.68 | A |
| ATOM | 260 | CA | GLY | A | 50 | −27.393 | −2.474 | 16.186 | 1.00 | 57.76 | A |
| ATOM | 261 | C | GLY | A | 50 | −28.272 | −3.526 | 16.820 | 1.00 | 56.03 | A |
| ATOM | 262 | O | GLY | A | 50 | −28.624 | −4.524 | 16.196 | 1.00 | 52.94 | A |
| ATOM | 263 | N | LYS | A | 51 | −28.617 | −3.296 | 18.082 | 1.00 | 56.72 | A |
| ATOM | 264 | CA | LYS | A | 51 | −29.532 | −4.168 | 18.797 | 1.00 | 54.69 | A |
| ATOM | 265 | CB | LYS | A | 51 | −29.389 | −4.007 | 20.309 | 1.00 | 55.22 | A |
| ATOM | 266 | CG | LYS | A | 51 | −28.187 | −4.700 | 20.910 | 1.00 | 57.57 | A |
| ATOM | 267 | CD | LYS | A | 51 | −28.250 | −6.206 | 20.697 | 1.00 | 59.89 | A |
| ATOM | 268 | CE | LYS | A | 51 | −27.243 | −6.916 | 21.577 | 1.00 | 60.88 | A |
| ATOM | 269 | NZ | LYS | A | 51 | −25.879 | −6.385 | 21.356 | 1.00 | 62.43 | A |
| ATOM | 270 | C | LYS | A | 51 | −30.900 | −3.653 | 18.356 | 1.00 | 53.76 | A |
| ATOM | 271 | O | LYS | A | 51 | −31.930 | −4.156 | 18.794 | 1.00 | 52.56 | A |
| ATOM | 272 | N | LEU | A | 52 | −30.893 | −2.611 | 17.520 | 1.00 | 52.57 | A |
| ATOM | 273 | CA | LEU | A | 52 | −32.128 | −2.059 | 16.971 | 1.00 | 52.52 | A |
| ATOM | 274 | CB | LEU | A | 52 | −31.995 | −.561 | 16.701 | 1.00 | 50.30 | A |
| ATOM | 275 | CG | LEU | A | 52 | −31.785 | .378 | 17.886 | 1.00 | 49.22 | A |
| ATOM | 276 | CD1 | LEU | A | 52 | −31.916 | 1.828 | 17.429 | 1.00 | 48.30 | A |
| ATOM | 277 | CD2 | LEU | A | 52 | −32.807 | .086 | 18.950 | 1.00 | 48.33 | A |
| ATOM | 278 | C | LEU | A | 52 | −32.331 | −2.820 | 15.660 | 1.00 | 52.56 | A |
| ATOM | 279 | O | LEU | A | 52 | −31.553 | −2.665 | 14.723 | 1.00 | 52.15 | A |
| ATOM | 280 | N | ARG | A | 53 | −33.381 | −3.636 | 15.598 | 1.00 | 53.98 | A |
| ATOM | 281 | CA | ARG | A | 53 | −33.621 | −4.472 | 14.423 | 1.00 | 55.47 | A |
| ATOM | 282 | CB | ARG | A | 53 | −32.944 | −5.813 | 14.675 | 1.00 | 55.68 | A |
| ATOM | 283 | CG | ARG | A | 53 | −32.740 | −6.710 | 13.493 | 1.00 | 58.73 | A |
| ATOM | 284 | CD | ARG | A | 53 | −32.307 | −8.067 | 14.025 | 1.00 | 64.14 | A |
| ATOM | 285 | NE | ARG | A | 53 | −31.766 | −8.956 | 13.003 | 1.00 | 69.14 | A |
| ATOM | 286 | CZ | ARG | A | 53 | −30.543 | −8.847 | 12.492 | 1.00 | 72.43 | A |
| ATOM | 287 | NH1 | ARG | A | 53 | −29.731 | −7.877 | 12.918 | 1.00 | 73.04 | A |
| ATOM | 288 | NH2 | ARG | A | 53 | −30.132 | −9.704 | 11.554 | 1.00 | 71.68 | A |
| ATOM | 289 | C | ARG | A | 53 | −35.105 | −4.699 | 14.097 | 1.00 | 56.16 | A |
| ATOM | 290 | O | ARG | A | 53 | −35.700 | −5.667 | 14.567 | 1.00 | 57.02 | A |
| ATOM | 291 | N | SER | A | 54 | −35.693 | −3.829 | 13.274 | 1.00 | 55.95 | A |
| ATOM | 292 | CA | SER | A | 54 | −37.107 | −3.956 | 12.908 | 1.00 | 54.97 | A |
| ATOM | 293 | CB | SER | A | 54 | −37.702 | −2.575 | 12.646 | 1.00 | 54.82 | A |
| ATOM | 294 | OG | SER | A | 54 | −37.340 | −1.666 | 13.672 | 1.00 | 55.67 | A |
| ATOM | 295 | C | SER | A | 54 | −37.309 | −4.822 | 11.674 | 1.00 | 54.36 | A |
| ATOM | 296 | O | SER | A | 54 | −36.498 | −4.797 | 10.761 | 1.00 | 56.15 | A |
| ATOM | 297 | N | VAL | A | 55 | −38.395 | −5.585 | 11.647 | 1.00 | 54.56 | A |
| ATOM | 298 | CA | VAL | A | 55 | −38.694 | −6.450 | 10.505 | 1.00 | 54.77 | A |
| ATOM | 299 | CB | VAL | A | 55 | −38.528 | −7.934 | 10.860 | 1.00 | 55.34 | A |
| ATOM | 300 | CG1 | VAL | A | 55 | −39.257 | −8.797 | 9.838 | 1.00 | 55.38 | A |
| ATOM | 301 | CG2 | VAL | A | 55 | −37.061 | −8.294 | 10.880 | 1.00 | 55.13 | A |
| ATOM | 302 | C | VAL | A | 55 | −40.102 | −6.251 | 9.952 | 1.00 | 55.08 | A |
| ATOM | 303 | O | VAL | A | 55 | −41.060 | −6.085 | 10.699 | 1.00 | 53.28 | A |
| ATOM | 304 | N | SER | A | 56 | −40.215 | −6.288 | 8.629 | 1.00 | 57.44 | A |
| ATOM | 305 | CA | SER | A | 56 | −41.496 | −6.104 | 7.956 | 1.00 | 58.93 | A |
| ATOM | 306 | CB | SER | A | 56 | −41.401 | −4.942 | 6.978 | 1.00 | 59.66 | A |
| ATOM | 307 | OG | SER | A | 56 | −42.672 | −4.677 | 6.422 | 1.00 | 64.28 | A |
| ATOM | 308 | C | SER | A | 56 | −41.924 | −7.356 | 7.206 | 1.00 | 58.61 | A |
| ATOM | 309 | O | SER | A | 56 | −41.188 | −7.875 | 6.380 | 1.00 | 56.89 | A |
| ATOM | 310 | N | GLN | A | 57 | −43.119 | −7.846 | 7.486 | 1.00 | 60.38 | A |
| ATOM | 311 | CA | GLN | A | 57 | −43.559 | −9.043 | 6.807 | 1.00 | 64.49 | A |
| ATOM | 312 | CB | GLN | A | 57 | −43.290 | −10.281 | 7.663 | 1.00 | 67.79 | A |
| ATOM | 313 | CG | GLN | A | 57 | −41.851 | −10.450 | 8.144 | 1.00 | 72.98 | A |
| ATOM | 314 | CD | GLN | A | 57 | −40.934 | −11.054 | 7.105 | 1.00 | 73.47 | A |
| ATOM | 315 | OE1 | GLN | A | 57 | −40.745 | −10.486 | 6.028 | 1.00 | 74.82 | A |
| ATOM | 316 | NE2 | GLN | A | 57 | −40.351 | −12.211 | 7.426 | 1.00 | 71.52 | A |
| ATOM | 317 | C | GLN | A | 57 | −45.030 | −9.028 | 6.453 | 1.00 | 66.12 | A |
| ATOM | 318 | O | GLN | A | 57 | −45.647 | −7.971 | 6.270 | 1.00 | 65.80 | A |
| ATOM | 319 | N | ASP | A | 58 | −45.563 | −10.244 | 6.360 | 1.00 | 66.86 | A |
| ATOM | 320 | CA | ASP | A | 58 | −46.945 | −10.517 | 6.023 | 1.00 | 66.59 | A |
| ATOM | 321 | CB | ASP | A | 58 | −47.201 | −12.020 | 6.148 | 1.00 | 68.22 | A |
| ATOM | 322 | CG | ASP | A | 58 | −45.936 | −12.802 | 6.544 | 1.00 | 70.64 | A |
| ATOM | 323 | OD1 | ASP | A | 58 | −44.906 | −12.715 | 5.827 | 1.00 | 70.06 | A |
| ATOM | 324 | OD2 | ASP | A | 58 | −45.972 | −13.508 | 7.579 | 1.00 | 72.26 | A |
| ATOM | 325 | C | ASP | A | 58 | −47.880 | −9.737 | 6.926 | 1.00 | 66.93 | A |
| ATOM | 326 | O | ASP | A | 58 | −48.382 | −10.259 | 7.931 | 1.00 | 66.12 | A |
| ATOM | 327 | N | GLY | A | 59 | −48.096 | −8.475 | 6.552 | 1.00 | 67.01 | A |
| ATOM | 328 | CA | GLY | A | 59 | −48.977 | −7.590 | 7.296 | 1.00 | 66.08 | A |
| ATOM | 329 | C | GLY | A | 59 | −48.564 | −7.371 | 8.733 | 1.00 | 64.96 | A |
| ATOM | 330 | O | GLY | A | 59 | −49.388 | −6.986 | 9.565 | 1.00 | 64.17 | A |
| ATOM | 331 | N | LEU | A | 60 | −47.289 | −7.616 | 9.025 | 1.00 | 63.97 | A |
| ATOM | 332 | CA | LEU | A | 60 | −46.767 | −7.449 | 10.373 | 1.00 | 63.01 | A |
| ATOM | 333 | CB | LEU | A | 60 | −46.342 | −8.800 | 10.958 | 1.00 | 62.03 | A |
| ATOM | 334 | CG | LEU | A | 60 | −47.343 | −9.956 | 10.932 | 1.00 | 61.98 | A |
| ATOM | 335 | CD1 | LEU | A | 60 | −46.686 | −11.198 | 11.503 | 1.00 | 60.69 | A |
| ATOM | 336 | CD2 | LEU | A | 60 | −48.600 | −9.586 | 11.722 | 1.00 | 61.48 | A |
| ATOM | 337 | C | LEU | A | 60 | −45.559 | −6.534 | 10.375 | 1.00 | 62.90 | A |
| ATOM | 338 | O | LEU | A | 60 | −44.844 | −6.420 | 9.382 | 1.00 | 64.55 | A |
| ATOM | 339 | N | ILE | A | 61 | −45.339 | −5.885 | 11.507 | 1.00 | 62.16 | A |
| ATOM | 340 | CA | ILE | A | 61 | −44.189 | −5.015 | 11.698 | 1.00 | 61.09 | A |
| ATOM | 341 | CB | ILE | A | 61 | −44.555 | −3.529 | 11.564 | 1.00 | 59.98 | A |
| ATOM | 342 | CG2 | ILE | A | 61 | −43.369 | −2.660 | 11.967 | 1.00 | 59.85 | A |
| ATOM | 343 | CG1 | ILE | A | 61 | −44.985 | −3.226 | 10.132 | 1.00 | 58.02 | A |
| ATOM | 344 | CD1 | ILE | A | 61 | −45.215 | −1.753 | 9.875 | 1.00 | 57.26 | A |
| ATOM | 345 | C | ILE | A | 61 | −43.752 | −5.290 | 13.127 | 1.00 | 61.47 | A |
| ATOM | 346 | O | ILE | A | 61 | −44.525 | −5.084 | 14.066 | 1.00 | 62.18 | A |
| ATOM | 347 | N | TRP | A | 62 | −42.531 | −5.781 | 13.301 | 1.00 | 61.78 | A |
| ATOM | 348 | CA | TRP | A | 62 | −42.055 | −6.066 | 14.644 | 1.00 | 61.55 | A |
| ATOM | 349 | CB | TRP | A | 62 | −42.372 | −7.517 | 15.022 | 1.00 | 63.23 | A |
| ATOM | 350 | CG | TRP | A | 62 | −41.438 | −8.549 | 14.487 | 1.00 | 65.03 | A |
| ATOM | 351 | CD2 | TRP | A | 62 | −41.630 | −9.353 | 13.322 | 1.00 | 65.69 | A |
| ATOM | 352 | CE2 | TRP | A | 62 | −40.519 | −10.217 | 13.223 | 1.00 | 67.98 | A |
| ATOM | 353 | CE3 | TRP | A | 62 | −42.636 | −9.428 | 12.349 | 1.00 | 65.48 | A |
| ATOM | 354 | CD1 | TRP | A | 62 | −40.252 | −8.941 | 15.032 | 1.00 | 67.59 | A |
| ATOM | 355 | NE1 | TRP | A | 62 | −39.691 | −9.947 | 14.281 | 1.00 | 68.61 | A |
| ATOM | 356 | CZ2 | TRP | A | 62 | −40.383 | −11.150 | 12.184 | 1.00 | 69.06 | A |
| ATOM | 357 | CZ3 | TRP | A | 62 | −42.505 | −10.349 | 11.320 | 1.00 | 65.50 | A |
| ATOM | 358 | CH2 | TRP | A | 62 | −41.386 | −11.199 | 11.244 | 1.00 | 67.46 | A |
| ATOM | 359 | C | TRP | A | 62 | −40.583 | −5.782 | 14.858 | 1.00 | 59.91 | A |
| ATOM | 360 | O | TRP | A | 62 | −39.810 | −5.647 | 13.908 | 1.00 | 59.02 | A |
| ATOM | 361 | N | ASP | A | 63 | −40.216 | −5.677 | 16.129 | 1.00 | 58.27 | A |
| ATOM | 362 | CA | ASP | A | 63 | −38.844 | −5.425 | 16.516 | 1.00 | 56.93 | A |
| ATOM | 363 | CB | ASP | A | 63 | −38.779 | −4.232 | 17.465 | 1.00 | 55.40 | A |
| ATOM | 364 | CG | ASP | A | 63 | −38.675 | −2.916 | 16.725 | 1.00 | 56.09 | A |
| ATOM | 365 | OD1 | ASP | A | 63 | −37.678 | −2.744 | 15.990 | 1.00 | 57.39 | A |
| ATOM | 366 | OD2 | ASP | A | 63 | −39.577 | −2.058 | 16.870 | 1.00 | 53.77 | A |
| ATOM | 367 | C | ASP | A | 63 | −38.273 | −6.669 | 17.176 | 1.00 | 56.35 | A |
| ATOM | 368 | O | ASP | A | 63 | −38.855 | −7.213 | 18.108 | 1.00 | 57.70 | A |
| ATOM | 369 | N | GLU | A | 64 | −37.136 | −7.126 | 16.676 | 1.00 | 55.08 | A |
| ATOM | 370 | CA | GLU | A | 64 | −36.499 | −8.306 | 17.216 | 1.00 | 53.97 | A |
| ATOM | 371 | CB | GLU | A | 64 | −35.757 | −9.032 | 16.098 | 1.00 | 56.51 | A |
| ATOM | 372 | CG | GLU | A | 64 | −36.615 | −9.266 | 14.860 | 1.00 | 59.80 | A |
| ATOM | 373 | CD | GLU | A | 64 | −35.895 | −10.078 | 13.789 | 1.00 | 62.72 | A |
| ATOM | 374 | OE1 | GLU | A | 64 | −34.936 | −9.560 | 13.171 | 1.00 | 61.47 | A |
| ATOM | 375 | OE2 | GLU | A | 64 | −36.293 | −11.245 | 13.574 | 1.00 | 63.89 | A |
| ATOM | 376 | C | GLU | A | 64 | −35.543 | −7.905 | 18.330 | 1.00 | 51.47 | A |
| ATOM | 377 | O | GLU | A | 64 | −35.026 | −8.748 | 19.050 | 1.00 | 51.83 | A |
| ATOM | 378 | N | GLY | A | 65 | −35.316 | −6.607 | 18.466 | 1.00 | 48.99 | A |
| ATOM | 379 | CA | GLY | A | 65 | −34.425 | −6.119 | 19.496 | 1.00 | 47.83 | A |
| ATOM | 380 | C | GLY | A | 65 | −35.139 | −5.091 | 20.340 | 1.00 | 47.53 | A |
| ATOM | 381 | O | GLY | A | 65 | −36.237 | −5.340 | 20.825 | 1.00 | 47.14 | A |
| ATOM | 382 | N | ALA | A | 66 | −34.528 | −3.931 | 20.526 | 1.00 | 47.71 | A |
| ATOM | 383 | CA | ALA | A | 66 | −35.169 | −2.892 | 21.314 | 1.00 | 48.09 | A |
| ATOM | 384 | CB | ALA | A | 66 | −34.317 | −1.649 | 21.323 | 1.00 | 46.89 | A |
| ATOM | 385 | C | ALA | A | 66 | −36.548 | −2.590 | 20.731 | 1.00 | 49.68 | A |
| ATOM | 386 | O | ALA | A | 66 | −36.695 | −2.415 | 19.525 | 1.00 | 48.69 | A |
| ATOM | 387 | N | ASN | A | 67 | −37.560 | −2.530 | 21.593 | 1.00 | 52.21 | A |
| ATOM | 388 | CA | ASN | A | 67 | −38.933 | −2.263 | 21.159 | 1.00 | 52.88 | A |
| ATOM | 389 | CB | ASN | A | 67 | −39.889 | −3.228 | 21.845 | 1.00 | 52.31 | A |
| ATOM | 390 | CG | ASN | A | 67 | −40.278 | −4.367 | 20.959 | 1.00 | 54.66 | A |
| ATOM | 391 | OD1 | ASN | A | 67 | −41.337 | −4.342 | 20.329 | 1.00 | 56.38 | A |
| ATOM | 392 | ND2 | ASN | A | 67 | −39.415 | −5.374 | 20.877 | 1.00 | 55.29 | A |
| ATOM | 393 | C | ASN | A | 67 | −39.415 | −.851 | 21.413 | 1.00 | 53.04 | A |
| ATOM | 394 | O | ASN | A | 67 | −40.296 | −.349 | 20.722 | 1.00 | 54.00 | A |
| ATOM | 395 | N | THR | A | 68 | −38.836 | −.209 | 22.411 | 1.00 | 53.19 | A |
| ATOM | 396 | CA | THR | A | 68 | −39.245 | 1.128 | 22.760 | 1.00 | 53.79 | A |
| ATOM | 397 | CB | THR | A | 68 | −40.401 | 1.066 | 23.800 | 1.00 | 55.03 | A |
| ATOM | 398 | OG1 | THR | A | 68 | −40.940 | 2.376 | 23.996 | 1.00 | 57.24 | A |
| ATOM | 399 | CG2 | THR | A | 68 | −39.918 | .489 | 25.136 | 1.00 | 54.79 | A |
| ATOM | 400 | C | THR | A | 68 | −38.033 | 1.875 | 23.295 | 1.00 | 54.56 | A |
| ATOM | 401 | O | THR | A | 68 | −36.968 | 1.293 | 23.481 | 1.00 | 54.25 | A |
| ATOM | 402 | N | MSE | A | 69 | −38.194 | 3.165 | 23.539 | 1.00 | 56.48 | A |
| ATOM | 403 | CA | MSE | A | 69 | −37.092 | 3.991 | 24.001 | 1.00 | 59.20 | A |
| ATOM | 404 | CB | MSE | A | 69 | −36.210 | 4.337 | 22.800 | 1.00 | 61.93 | A |
| ATOM | 405 | CG | MSE | A | 69 | −35.312 | 5.553 | 22.959 | 1.00 | 68.20 | A |
| ATOM | 406 | SE | MSE | A | 69 | −34.929 | 6.353 | 21.230 | 1.00 | 72.30 | A |
| ATOM | 407 | CE | MSE | A | 69 | −36.321 | 5.451 | 20.268 | 1.00 | 70.71 | A |
| ATOM | 408 | C | MSE | A | 69 | −37.646 | 5.252 | 24.646 | 1.00 | 59.18 | A |
| ATOM | 409 | O | MSE | A | 69 | −38.839 | 5.508 | 24.578 | 1.00 | 58.44 | A |
| ATOM | 410 | N | THR | A | 70 | −36.778 | 6.036 | 25.269 | 1.00 | 60.00 | A |
| ATOM | 411 | CA | THR | A | 70 | −37.199 | 7.258 | 25.937 | 1.00 | 61.97 | A |
| ATOM | 412 | CB | THR | A | 70 | −37.274 | 7.047 | 27.463 | 1.00 | 59.99 | A |
| ATOM | 413 | OG1 | THR | A | 70 | −38.255 | 6.056 | 27.758 | 1.00 | 58.12 | A |
| ATOM | 414 | CG2 | THR | A | 70 | −37.647 | 8.331 | 28.166 | 1.00 | 61.55 | A |
| ATOM | 415 | C | THR | A | 70 | −36.195 | 8.363 | 25.681 | 1.00 | 64.20 | A |
| ATOM | 416 | O | THR | A | 70 | −35.066 | 8.091 | 25.275 | 1.00 | 65.78 | A |
| ATOM | 417 | N | GLU | A | 71 | −36.593 | 9.610 | 25.914 | 1.00 | 65.95 | A |
| ATOM | 418 | CA | GLU | A | 71 | −35.654 | 10.702 | 25.746 | 1.00 | 68.21 | A |
| ATOM | 419 | CB | GLU | A | 71 | −36.099 | 11.641 | 24.626 | 1.00 | 68.63 | A |
| ATOM | 420 | CG | GLU | A | 71 | −37.256 | 12.539 | 24.911 | 1.00 | 70.30 | A |
| ATOM | 421 | CD | GLU | A | 71 | −37.639 | 13.338 | 23.678 | 1.00 | 72.84 | A |
| ATOM | 422 | OE1 | GLU | A | 71 | −38.382 | 12.785 | 22.827 | 1.00 | 73.43 | A |
| ATOM | 423 | OE2 | GLU | A | 71 | −37.178 | 14.503 | 23.553 | 1.00 | 70.75 | A |
| ATOM | 424 | C | GLU | A | 71 | −35.433 | 11.450 | 27.061 | 1.00 | 69.28 | A |
| ATOM | 425 | O | GLU | A | 71 | −35.789 | 12.614 | 27.217 | 1.00 | 69.63 | A |
| ATOM | 426 | N | SER | A | 72 | −34.838 | 10.739 | 28.011 | 1.00 | 70.41 | A |
| ATOM | 427 | CA | SER | A | 72 | −34.522 | 11.275 | 29.323 | 1.00 | 70.86 | A |
| ATOM | 428 | CB | SER | A | 72 | −34.546 | 10.158 | 30.362 | 1.00 | 71.28 | A |
| ATOM | 429 | OG | SER | A | 72 | −35.571 | 9.224 | 30.070 | 1.00 | 72.44 | A |
| ATOM | 430 | C | SER | A | 72 | −33.105 | 11.809 | 29.210 | 1.00 | 71.71 | A |
| ATOM | 431 | O | SER | A | 72 | −32.559 | 12.375 | 30.156 | 1.00 | 71.81 | A |
| ATOM | 432 | N | GLU | A | 73 | −32.508 | 11.603 | 28.042 | 1.00 | 72.44 | A |
| ATOM | 433 | CA | GLU | A | 73 | −31.150 | 12.051 | 27.796 | 1.00 | 73.72 | A |
| ATOM | 434 | CB | GLU | A | 73 | −30.322 | 10.920 | 27.182 | 1.00 | 76.26 | A |
| ATOM | 435 | CG | GLU | A | 73 | −30.320 | 9.630 | 27.988 | 1.00 | 78.56 | A |
| ATOM | 436 | CD | GLU | A | 73 | −29.787 | 9.824 | 29.398 | 1.00 | 81.16 | A |
| ATOM | 437 | OE1 | GLU | A | 73 | −28.604 | 10.200 | 29.545 | 1.00 | 81.56 | A |
| ATOM | 438 | OE2 | GLU | A | 73 | −30.557 | 9.601 | 30.363 | 1.00 | 83.52 | A |
| ATOM | 439 | C | GLU | A | 73 | −31.207 | 13.221 | 26.837 | 1.00 | 73.50 | A |
| ATOM | 440 | O | GLU | A | 73 | −31.992 | 13.201 | 25.886 | 1.00 | 74.40 | A |
| ATOM | 441 | N | GLY | A | 74 | −30.380 | 14.235 | 27.087 | 1.00 | 71.99 | A |
| ATOM | 442 | CA | GLY | A | 74 | −30.361 | 15.402 | 26.226 | 1.00 | 70.32 | A |
| ATOM | 443 | C | GLY | A | 74 | −30.019 | 15.022 | 24.799 | 1.00 | 69.94 | A |
| ATOM | 444 | O | GLY | A | 74 | −30.651 | 15.485 | 23.836 | 1.00 | 70.17 | A |
| ATOM | 445 | N | ASP | A | 75 | −29.021 | 14.153 | 24.669 | 1.00 | 68.57 | A |
| ATOM | 446 | CA | ASP | A | 75 | −28.564 | 13.699 | 23.367 | 1.00 | 66.63 | A |
| ATOM | 447 | CB | ASP | A | 75 | −27.383 | 12.754 | 23.518 | 1.00 | 67.38 | A |
| ATOM | 448 | CG | ASP | A | 75 | −26.122 | 13.470 | 23.925 | 1.00 | 67.93 | A |
| ATOM | 449 | OD1 | ASP | A | 75 | −25.765 | 14.470 | 23.253 | 1.00 | 68.54 | A |
| ATOM | 450 | OD2 | ASP | A | 75 | −25.485 | 13.028 | 24.907 | 1.00 | 68.05 | A |
| ATOM | 451 | C | ASP | A | 75 | −29.630 | 13.027 | 22.532 | 1.00 | 65.14 | A |
| ATOM | 452 | O | ASP | A | 75 | −29.555 | 13.069 | 21.308 | 1.00 | 65.07 | A |
| ATOM | 453 | N | VAL | A | 76 | −30.610 | 12.400 | 23.176 | 1.00 | 63.50 | A |
| ATOM | 454 | CA | VAL | A | 76 | −31.676 | 11.741 | 22.432 | 1.00 | 62.78 | A |
| ATOM | 455 | CB | VAL | A | 76 | −32.556 | 10.896 | 23.328 | 1.00 | 62.46 | A |
| ATOM | 456 | CG1 | VAL | A | 76 | −33.714 | 10.314 | 22.511 | 1.00 | 61.19 | A |
| ATOM | 457 | CG2 | VAL | A | 76 | −31.732 | 9.808 | 23.965 | 1.00 | 64.22 | A |
| ATOM | 458 | C | VAL | A | 76 | −32.569 | 12.780 | 21.782 | 1.00 | 63.29 | A |
| ATOM | 459 | O | VAL | A | 76 | −32.965 | 12.638 | 20.630 | 1.00 | 62.22 | A |
| ATOM | 460 | N | THR | A | 77 | −32.886 | 13.826 | 22.539 | 1.00 | 64.07 | A |
| ATOM | 461 | CA | THR | A | 77 | −33.736 | 14.902 | 22.049 | 1.00 | 63.73 | A |
| ATOM | 462 | CB | THR | A | 77 | −34.027 | 15.911 | 23.164 | 1.00 | 64.24 | A |
| ATOM | 463 | OG1 | THR | A | 77 | −34.746 | 15.248 | 24.216 | 1.00 | 64.34 | A |
| ATOM | 464 | CG2 | THR | A | 77 | −34.852 | 17.081 | 22.629 | 1.00 | 63.54 | A |
| ATOM | 465 | C | THR | A | 77 | −33.039 | 15.598 | 20.894 | 1.00 | 63.27 | A |
| ATOM | 466 | O | THR | A | 77 | −33.657 | 15.872 | 19.852 | 1.00 | 62.03 | A |
| ATOM | 467 | N | PHE | A | 78 | −31.749 | 15.872 | 21.091 | 1.00 | 62.02 | A |
| ATOM | 468 | CA | PHE | A | 78 | −30.929 | 16.504 | 20.069 | 1.00 | 61.44 | A |
| ATOM | 469 | CB | PHE | A | 78 | −29.485 | 16.640 | 20.553 | 1.00 | 63.68 | A |
| ATOM | 470 | CG | PHE | A | 78 | −28.507 | 16.982 | 19.457 | 1.00 | 66.00 | A |
| ATOM | 471 | CD1 | PHE | A | 78 | −28.059 | 16.005 | 18.572 | 1.00 | 66.94 | A |
| ATOM | 472 | CD2 | PHE | A | 78 | −28.071 | 18.293 | 19.277 | 1.00 | 67.54 | A |
| ATOM | 473 | CE1 | PHE | A | 78 | −27.199 | 16.327 | 17.523 | 1.00 | 67.61 | A |
| ATOM | 474 | CE2 | PHE | A | 78 | −27.210 | 18.626 | 18.230 | 1.00 | 68.15 | A |
| ATOM | 475 | CZ | PHE | A | 78 | −26.775 | 17.638 | 17.351 | 1.00 | 67.92 | A |
| ATOM | 476 | C | PHE | A | 78 | −30.956 | 15.703 | 18.776 | 1.00 | 60.74 | A |
| ATOM | 477 | O | PHE | A | 78 | −31.240 | 16.252 | 17.725 | 1.00 | 59.98 | A |
| ATOM | 478 | N | LEU | A | 79 | −30.656 | 14.410 | 18.848 | 1.00 | 60.37 | A |
| ATOM | 479 | CA | LEU | A | 79 | −30.656 | 13.586 | 17.652 | 1.00 | 62.21 | A |
| ATOM | 480 | CB | LEU | A | 79 | −30.137 | 12.189 | 17.950 | 1.00 | 62.71 | A |
| ATOM | 481 | CG | LEU | A | 79 | −28.641 | 12.077 | 18.220 | 1.00 | 64.56 | A |
| ATOM | 482 | CD1 | LEU | A | 79 | −28.285 | 10.602 | 18.308 | 1.00 | 65.93 | A |
| ATOM | 483 | CD2 | LEU | A | 79 | −27.841 | 12.752 | 17.108 | 1.00 | 65.48 | A |
| ATOM | 484 | C | LEU | A | 79 | −32.013 | 13.472 | 16.985 | 1.00 | 64.00 | A |
| ATOM | 485 | O | LEU | A | 79 | −32.102 | 13.513 | 15.756 | 1.00 | 65.06 | A |
| ATOM | 486 | N | ILE | A | 80 | −33.070 | 13.310 | 17.779 | 1.00 | 65.46 | A |
| ATOM | 487 | CA | ILE | A | 80 | −34.412 | 13.208 | 17.218 | 1.00 | 65.41 | A |
| ATOM | 488 | CB | ILE | A | 80 | −35.498 | 13.164 | 18.304 | 1.00 | 65.05 | A |
| ATOM | 489 | CG2 | ILE | A | 80 | −36.865 | 13.156 | 17.648 | 1.00 | 66.12 | A |
| ATOM | 490 | CG1 | ILE | A | 80 | −35.346 | 11.915 | 19.170 | 1.00 | 64.87 | A |
| ATOM | 491 | CD1 | ILE | A | 80 | −35.607 | 10.620 | 18.450 | 1.00 | 63.78 | A |
| ATOM | 492 | C | ILE | A | 80 | −34.677 | 14.428 | 16.349 | 1.00 | 65.82 | A |
| ATOM | 493 | O | ILE | A | 80 | −35.310 | 14.321 | 15.301 | 1.00 | 65.27 | A |
| ATOM | 494 | N | ASP | A | 81 | −34.188 | 15.586 | 16.786 | 1.00 | 66.55 | A |
| ATOM | 495 | CA | ASP | A | 81 | −34.380 | 16.825 | 16.031 | 1.00 | 68.29 | A |
| ATOM | 496 | CB | ASP | A | 81 | −34.167 | 18.057 | 16.919 | 1.00 | 70.46 | A |
| ATOM | 497 | CG | ASP | A | 81 | −35.148 | 18.127 | 18.089 | 1.00 | 72.65 | A |
| ATOM | 498 | OD1 | ASP | A | 81 | −36.362 | 17.842 | 17.889 | 1.00 | 71.77 | A |
| ATOM | 499 | OD2 | ASP | A | 81 | −34.689 | 18.488 | 19.202 | 1.00 | 72.06 | A |
| ATOM | 500 | C | ASP | A | 81 | −33.467 | 16.939 | 14.819 | 1.00 | 67.58 | A |
| ATOM | 501 | O | ASP | A | 81 | −33.931 | 17.239 | 13.725 | 1.00 | 68.56 | A |
| ATOM | 502 | N | SER | A | 82 | −32.173 | 16.709 | 15.009 | 1.00 | 67.20 | A |
| ATOM | 503 | CA | SER | A | 82 | −31.227 | 16.801 | 13.905 | 1.00 | 66.56 | A |
| ATOM | 504 | CB | SER | A | 82 | −29.842 | 16.320 | 14.323 | 1.00 | 67.92 | A |
| ATOM | 505 | OG | SER | A | 82 | −29.190 | 17.279 | 15.145 | 1.00 | 70.51 | A |
| ATOM | 506 | C | SER | A | 82 | −31.699 | 15.982 | 12.741 | 1.00 | 66.23 | A |
| ATOM | 507 | O | SER | A | 82 | −31.940 | 16.510 | 11.669 | 1.00 | 67.32 | A |
| ATOM | 508 | N | LEU | A | 83 | −31.842 | 14.684 | 12.955 | 1.00 | 67.16 | A |
| ATOM | 509 | CA | LEU | A | 83 | −32.291 | 13.787 | 11.897 | 1.00 | 68.06 | A |
| ATOM | 510 | CB | LEU | A | 83 | −32.163 | 12.340 | 12.369 | 1.00 | 69.40 | A |
| ATOM | 511 | CG | LEU | A | 83 | −30.768 | 11.924 | 12.847 | 1.00 | 71.25 | A |
| ATOM | 512 | CD1 | LEU | A | 83 | −30.857 | 10.542 | 13.445 | 1.00 | 71.71 | A |
| ATOM | 513 | CD2 | LEU | A | 83 | −29.761 | 11.948 | 11.691 | 1.00 | 70.98 | A |
| ATOM | 514 | C | LEU | A | 83 | −33.728 | 14.064 | 11.464 | 1.00 | 68.14 | A |
| ATOM | 515 | O | LEU | A | 83 | −34.209 | 13.492 | 10.486 | 1.00 | 67.17 | A |
| ATOM | 516 | N | GLY | A | 84 | −34.401 | 14.949 | 12.200 | 1.00 | 69.67 | A |
| ATOM | 517 | CA | GLY | A | 84 | −35.783 | 15.298 | 11.902 | 1.00 | 69.83 | A |
| ATOM | 518 | C | GLY | A | 84 | −36.705 | 14.095 | 11.928 | 1.00 | 69.79 | A |
| ATOM | 519 | O | GLY | A | 84 | −37.383 | 13.821 | 10.946 | 1.00 | 70.35 | A |
| ATOM | 520 | N | LEU | A | 85 | −36.740 | 13.383 | 13.051 | 1.00 | 69.76 | A |
| ATOM | 521 | CA | LEU | A | 85 | −37.576 | 12.192 | 13.186 | 1.00 | 69.64 | A |
| ATOM | 522 | CB | LEU | A | 85 | −36.753 | 11.061 | 13.816 | 1.00 | 69.08 | A |
| ATOM | 523 | CG | LEU | A | 85 | −35.661 | 10.429 | 12.948 | 1.00 | 67.05 | A |
| ATOM | 524 | CD1 | LEU | A | 85 | −34.790 | 9.485 | 13.773 | 1.00 | 65.28 | A |
| ATOM | 525 | CD2 | LEU | A | 85 | −36.322 | 9.689 | 11.808 | 1.00 | 65.50 | A |
| ATOM | 526 | C | LEU | A | 85 | −38.880 | 12.397 | 13.976 | 1.00 | 70.00 | A |
| ATOM | 527 | O | LEU | A | 85 | −39.657 | 11.451 | 14.154 | 1.00 | 69.58 | A |
| ATOM | 528 | N | ARG | A | 86 | −39.121 | 13.626 | 14.434 | 1.00 | 69.83 | A |
| ATOM | 529 | CA | ARG | A | 86 | −40.334 | 13.954 | 15.183 | 1.00 | 70.53 | A |
| ATOM | 530 | CB | ARG | A | 86 | −40.476 | 15.470 | 15.331 | 1.00 | 70.47 | A |
| ATOM | 531 | CG | ARG | A | 86 | −39.501 | 16.103 | 16.298 | 1.00 | 71.16 | A |
| ATOM | 532 | CD | ARG | A | 86 | −39.682 | 15.525 | 17.692 | 1.00 | 72.10 | A |
| ATOM | 533 | NE | ARG | A | 86 | −38.770 | 16.112 | 18.671 | 1.00 | 70.70 | A |
| ATOM | 534 | CZ | ARG | A | 86 | −38.541 | 15.583 | 19.865 | 1.00 | 69.89 | A |
| ATOM | 535 | NH1 | ARG | A | 86 | −39.156 | 14.459 | 20.215 | 1.00 | 69.42 | A |
| ATOM | 536 | NH2 | ARG | A | 86 | −37.700 | 16.170 | 20.702 | 1.00 | 69.73 | A |
| ATOM | 537 | C | ARG | A | 86 | −41.615 | 13.400 | 14.555 | 1.00 | 70.97 | A |
| ATOM | 538 | O | ARG | A | 86 | −42.464 | 12.859 | 15.249 | 1.00 | 71.55 | A |
| ATOM | 539 | N | GLU | A | 87 | −41.767 | 13.550 | 13.246 | 1.00 | 72.21 | A |
| ATOM | 540 | CA | GLU | A | 87 | −42.959 | 13.048 | 12.567 | 1.00 | 73.06 | A |
| ATOM | 541 | CB | GLU | A | 87 | −42.998 | 13.530 | 11.107 | 1.00 | 74.69 | A |
| ATOM | 542 | CG | GLU | A | 87 | −43.089 | 15.049 | 10.937 | 1.00 | 76.01 | A |
| ATOM | 543 | CD | GLU | A | 87 | −43.080 | 15.478 | 9.483 | .00 | 75.55 | A |
| ATOM | 544 | OE1 | GLU | A | 87 | −43.153 | 16.697 | 9.223 | .00 | 75.59 | A |
| ATOM | 545 | OE2 | GLU | A | 87 | −43.001 | 14.598 | 8.600 | .00 | 75.59 | A |
| ATOM | 546 | C | GLU | A | 87 | −43.007 | 11.523 | 12.596 | 1.00 | 72.22 | A |
| ATOM | 547 | O | GLU | A | 87 | −44.083 | 10.925 | 12.564 | 1.00 | 72.74 | A |
| ATOM | 548 | N | LYS | A | 88 | −41.842 | 10.894 | 12.662 | 1.00 | 70.98 | A |
| ATOM | 549 | CA | LYS | A | 88 | −41.786 | 9.444 | 12.679 | 1.00 | 70.66 | A |
| ATOM | 550 | CB | LYS | A | 88 | −40.536 | 8.980 | 11.940 | 1.00 | 71.32 | A |
| ATOM | 551 | CG | LYS | A | 88 | −40.618 | 9.263 | 10.450 | 1.00 | 71.38 | A |
| ATOM | 552 | CD | LYS | A | 88 | −39.342 | 8.899 | 9.728 | 1.00 | 72.18 | A |
| ATOM | 553 | CE | LYS | A | 88 | −39.500 | 9.053 | 8.225 | 1.00 | 71.58 | A |
| ATOM | 554 | NZ | LYS | A | 88 | −38.286 | 8.602 | 7.481 | 1.00 | 73.74 | A |
| ATOM | 555 | C | LYS | A | 88 | −41.850 | 8.844 | 14.080 | 1.00 | 70.47 | A |
| ATOM | 556 | O | LYS | A | 88 | −42.009 | 7.627 | 14.236 | 1.00 | 69.03 | A |
| ATOM | 557 | N | GLN | A | 89 | −41.743 | 9.707 | 15.091 | 1.00 | 70.07 | A |
| ATOM | 558 | CA | GLN | A | 89 | −41.812 | 9.284 | 16.488 | 1.00 | 69.71 | A |
| ATOM | 559 | CB | GLN | A | 89 | −41.524 | 10.465 | 17.411 | 1.00 | 68.68 | A |
| ATOM | 560 | CG | GLN | A | 89 | −41.157 | 10.083 | 18.823 | 1.00 | 70.07 | A |
| ATOM | 561 | CD | GLN | A | 89 | −40.862 | 11.293 | 19.695 | 1.00 | 71.64 | A |
| ATOM | 562 | OE1 | GLN | A | 89 | −40.418 | 11.157 | 20.835 | 1.00 | 72.93 | A |
| ATOM | 563 | NE2 | GLN | A | 89 | −41.113 | 12.486 | 19.162 | 1.00 | 71.03 | A |
| ATOM | 564 | C | GLN | A | 89 | −43.221 | 8.762 | 16.744 | 1.00 | 69.80 | A |
| ATOM | 565 | O | GLN | A | 89 | −44.200 | 9.453 | 16.479 | 1.00 | 70.56 | A |
| ATOM | 566 | N | GLN | A | 90 | −43.320 | 7.539 | 17.254 | 1.00 | 70.40 | A |
| ATOM | 567 | CA | GLN | A | 90 | −44.617 | 6.923 | 17.522 | 1.00 | 70.50 | A |
| ATOM | 568 | CB | GLN | A | 90 | −44.792 | 5.711 | 16.596 | 1.00 | 70.30 | A |
| ATOM | 569 | CG | GLN | A | 90 | −46.002 | 4.850 | 16.895 | 1.00 | 72.43 | A |
| ATOM | 570 | CD | GLN | A | 90 | −46.299 | 3.850 | 15.786 | 1.00 | 73.24 | A |
| ATOM | 571 | OE1 | GLN | A | 90 | −45.392 | 3.224 | 15.233 | 1.00 | 73.74 | A |
| ATOM | 572 | NE2 | GLN | A | 90 | −47.580 | 3.685 | 15.468 | 1.00 | 73.56 | A |
| ATOM | 573 | C | GLN | A | 90 | −44.833 | 6.520 | 18.992 | 1.00 | 69.43 | A |
| ATOM | 574 | O | GLN | A | 90 | −44.028 | 5.806 | 19.585 | 1.00 | 68.52 | A |
| ATOM | 575 | N | PHE | A | 91 | −45.925 | 6.996 | 19.574 | 1.00 | 68.71 | A |
| ATOM | 576 | CA | PHE | A | 91 | −46.240 | 6.673 | 20.954 | 1.00 | 68.85 | A |
| ATOM | 577 | CB | PHE | A | 91 | −46.738 | 7.907 | 21.692 | 1.00 | 68.47 | A |
| ATOM | 578 | CG | PHE | A | 91 | −45.730 | 9.022 | 21.753 | 1.00 | 68.14 | A |
| ATOM | 579 | CD1 | PHE | A | 91 | −45.495 | 9.828 | 20.646 | 1.00 | 66.43 | A |
| ATOM | 580 | CD2 | PHE | A | 91 | −45.001 | 9.257 | 22.923 | 1.00 | 67.72 | A |
| ATOM | 581 | CE1 | PHE | A | 91 | −44.557 | 10.846 | 20.700 | 1.00 | 65.71 | A |
| ATOM | 582 | CE2 | PHE | A | 91 | −44.060 | 10.273 | 22.985 | 1.00 | 66.39 | A |
| ATOM | 583 | CZ | PHE | A | 91 | −43.836 | 11.070 | 21.874 | 1.00 | 66.08 | A |
| ATOM | 584 | C | PHE | A | 91 | −47.299 | 5.586 | 20.995 | 1.00 | 69.47 | A |
| ATOM | 585 | O | PHE | A | 91 | −47.994 | 5.344 | 20.005 | 1.00 | 69.05 | A |
| ATOM | 586 | N | PRO | A | 92 | −47.425 | 4.896 | 22.138 | 1.00 | 69.85 | A |
| ATOM | 587 | CD | PRO | A | 92 | −46.535 | 4.888 | 23.310 | 1.00 | 68.24 | A |
| ATOM | 588 | CA | PRO | A | 92 | −48.430 | 3.830 | 22.232 | 1.00 | 69.98 | A |
| ATOM | 589 | CB | PRO | A | 92 | −48.063 | 3.121 | 23.532 | 1.00 | 70.25 | A |
| ATOM | 590 | CG | PRO | A | 92 | −46.584 | 3.445 | 23.700 | 1.00 | 69.93 | A |
| ATOM | 591 | C | PRO | A | 92 | −49.822 | 4.431 | 22.280 | 1.00 | 70.29 | A |
| ATOM | 592 | O | PRO | A | 92 | −50.027 | 5.475 | 22.901 | 1.00 | 71.15 | A |
| ATOM | 593 | N | ALA | A | 93 | −50.774 | 3.787 | 21.616 | 1.00 | 70.20 | A |
| ATOM | 594 | CA | ALA | A | 93 | −52.146 | 4.284 | 21.608 | 1.00 | 70.45 | A |
| ATOM | 595 | CB | ALA | A | 93 | −52.880 | 3.820 | 20.345 | 1.00 | 68.60 | A |
| ATOM | 596 | C | ALA | A | 93 | −52.859 | 3.769 | 22.852 | 1.00 | 71.22 | A |
| ATOM | 597 | O | ALA | A | 93 | −53.788 | 4.402 | 23.360 | 1.00 | 71.90 | A |
| ATOM | 598 | N | SER | A | 94 | −52.409 | 2.619 | 23.347 | 1.00 | 71.51 | A |
| ATOM | 599 | CA | SER | A | 94 | −53.004 | 2.017 | 24.526 | 1.00 | 70.72 | A |
| ATOM | 600 | CB | SER | A | 94 | −52.643 | .541 | 24.595 | 1.00 | 70.29 | A |
| ATOM | 601 | OG | SER | A | 94 | −51.246 | .393 | 24.691 | 1.00 | 71.67 | A |
| ATOM | 602 | C | SER | A | 94 | −52.541 | 2.714 | 25.795 | 1.00 | 71.02 | A |
| ATOM | 603 | O | SER | A | 94 | −51.478 | 3.334 | 25.832 | 1.00 | 69.93 | A |
| ATOM | 604 | N | GLN | A | 95 | −53.360 | 2.615 | 26.833 | 1.00 | 72.08 | A |
| ATOM | 605 | CA | GLN | A | 95 | −53.048 | 3.222 | 28.114 | 1.00 | 73.28 | A |
| ATOM | 606 | CB | GLN | A | 95 | −54.208 | 2.999 | 29.078 | 1.00 | 74.94 | A |
| ATOM | 607 | CG | GLN | A | 95 | −54.066 | 3.706 | 30.421 | 1.00 | 77.60 | A |
| ATOM | 608 | CD | GLN | A | 95 | −55.301 | 3.530 | 31.285 | 1.00 | 78.33 | A |
| ATOM | 609 | OE1 | GLN | A | 95 | −56.429 | 3.701 | 30.813 | 1.00 | 78.57 | A |
| ATOM | 610 | NE2 | GLN | A | 95 | −55.096 | 3.192 | 32.556 | 1.00 | 78.06 | A |
| ATOM | 611 | C | GLN | A | 95 | −51.790 | 2.577 | 28.670 | 1.00 | 73.08 | A |
| ATOM | 612 | O | GLN | A | 95 | −51.477 | 1.435 | 28.336 | 1.00 | 74.15 | A |
| ATOM | 613 | N | ASN | A | 96 | −51.056 | 3.302 | 29.505 | 1.00 | 72.09 | A |
| ATOM | 614 | CA | ASN | A | 96 | −49.855 | 2.726 | 30.088 | 1.00 | 71.01 | A |
| ATOM | 615 | CB | ASN | A | 96 | −48.852 | 3.817 | 30.455 | 1.00 | 71.98 | A |
| ATOM | 616 | CG | ASN | A | 96 | −49.172 | 4.470 | 31.772 | 1.00 | 72.63 | A |
| ATOM | 617 | OD1 | ASN | A | 96 | −50.288 | 4.953 | 31.982 | 1.00 | 73.02 | A |
| ATOM | 618 | ND2 | ASN | A | 96 | −48.203 | 4.477 | 32.679 | 1.00 | 73.47 | A |
| ATOM | 619 | C | ASN | A | 96 | −50.316 | 1.978 | 31.341 | 1.00 | 69.20 | A |
| ATOM | 620 | O | ASN | A | 96 | −50.634 | 2.581 | 32.367 | 1.00 | 70.02 | A |
| ATOM | 621 | N | LYS | A | 97 | −50.383 | .659 | 31.234 | 1.00 | 65.76 | A |
| ATOM | 622 | CA | LYS | A | 97 | −50.819 | −.185 | 32.335 | 1.00 | 61.96 | A |
| ATOM | 623 | CB | LYS | A | 97 | −52.310 | −.496 | 32.236 | 1.00 | 61.12 | A |
| ATOM | 624 | CG | LYS | A | 97 | −53.208 | .718 | 32.151 | 1.00 | 61.68 | A |
| ATOM | 625 | CD | LYS | A | 97 | −54.625 | .314 | 31.811 | 1.00 | 61.70 | A |
| ATOM | 626 | CE | LYS | A | 97 | −55.193 | −.610 | 32.869 | 1.00 | 64.52 | A |
| ATOM | 627 | NZ | LYS | A | 97 | −56.475 | −1.244 | 32.434 | 1.00 | 66.61 | A |
| ATOM | 628 | C | LYS | A | 97 | −50.063 | −1.461 | 32.143 | 1.00 | 60.58 | A |
| ATOM | 629 | O | LYS | A | 97 | −50.240 | −2.131 | 31.130 | 1.00 | 60.19 | A |
| ATOM | 630 | N | ARG | A | 98 | −49.212 | −1.788 | 33.107 | 1.00 | 58.66 | A |
| ATOM | 631 | CA | ARG | A | 98 | −48.421 | −3.001 | 33.047 | 1.00 | 56.99 | A |
| ATOM | 632 | CB | ARG | A | 98 | −46.984 | −2.702 | 33.468 | 1.00 | 58.72 | A |
| ATOM | 633 | CG | ARG | A | 98 | −46.311 | −1.633 | 32.622 | 1.00 | 61.26 | A |
| ATOM | 634 | CD | ARG | A | 98 | −44.953 | −1.232 | 33.193 | 1.00 | 65.50 | A |
| ATOM | 635 | NE | ARG | A | 98 | −44.147 | −.533 | 32.193 | 1.00 | 69.79 | A |
| ATOM | 636 | CZ | ARG | A | 98 | −44.325 | .731 | 31.816 | 1.00 | 71.32 | A |
| ATOM | 637 | NH1 | ARG | A | 98 | −45.283 | 1.469 | 32.365 | 1.00 | 72.98 | A |
| ATOM | 638 | NH2 | ARG | A | 98 | −43.563 | 1.248 | 30.857 | 1.00 | 70.41 | A |
| ATOM | 639 | C | ARG | A | 98 | −49.065 | −4.001 | 33.992 | 1.00 | 54.77 | A |
| ATOM | 640 | O | ARG | A | 98 | −49.808 | −3.616 | 34.886 | 1.00 | 54.23 | A |
| ATOM | 641 | N | TYR | A | 99 | −48.801 | −5.284 | 33.774 | 1.00 | 53.14 | A |
| ATOM | 642 | CA | TYR | A | 99 | −49.365 | −6.340 | 34.604 | 1.00 | 51.11 | A |
| ATOM | 643 | CB | TYR | A | 99 | −50.353 | −7.199 | 33.820 | 1.00 | 51.78 | A |
| ATOM | 644 | CG | TYR | A | 99 | −51.594 | −6.480 | 33.378 | 1.00 | 50.33 | A |
| ATOM | 645 | CD1 | TYR | A | 99 | −51.591 | −5.694 | 32.236 | 1.00 | 49.28 | A |
| ATOM | 646 | CE1 | TYR | A | 99 | −52.728 | −5.027 | 31.832 | 1.00 | 52.30 | A |
| ATOM | 647 | CD2 | TYR | A | 99 | −52.769 | −6.582 | 34.113 | 1.00 | 49.74 | A |
| ATOM | 648 | CE2 | TYR | A | 99 | −53.915 | −5.918 | 33.725 | 1.00 | 51.63 | A |
| ATOM | 649 | CZ | TYR | A | 99 | −53.895 | −5.141 | 32.582 | 1.00 | 53.63 | A |
| ATOM | 650 | OH | TYR | A | 99 | −55.043 | −4.476 | 32.192 | 1.00 | 54.41 | A |
| ATOM | 651 | C | TYR | A | 99 | −48.269 | −7.233 | 35.089 | 1.00 | 49.90 | A |
| ATOM | 652 | O | TYR | A | 99 | −47.238 | −7.353 | 34.439 | 1.00 | 50.09 | A |
| ATOM | 653 | N | ILE | A | 100 | −48.498 | −7.872 | 36.228 | 1.00 | 49.18 | A |
| ATOM | 654 | CA | ILE | A | 100 | −47.508 | −8.770 | 36.784 | 1.00 | 49.80 | A |
| ATOM | 655 | CB | ILE | A | 100 | −47.011 | −8.295 | 38.167 | 1.00 | 49.62 | A |
| ATOM | 656 | CG2 | ILE | A | 100 | −45.763 | −9.054 | 38.547 | 1.00 | 50.44 | A |
| ATOM | 657 | CG1 | ILE | A | 100 | −46.682 | −6.798 | 38.144 | 1.00 | 49.63 | A |
| ATOM | 658 | CD1 | ILE | A | 100 | −45.457 | −6.419 | 37.349 | 1.00 | 48.91 | A |
| ATOM | 659 | C | ILE | A | 100 | −48.222 | −10.088 | 36.960 | 1.00 | 49.85 | A |
| ATOM | 660 | O | ILE | A | 100 | −49.397 | −10.101 | 37.282 | 1.00 | 50.10 | A |
| ATOM | 661 | N | ALA | A | 101 | −47.533 | −11.195 | 36.726 | 1.00 | 50.67 | A |
| ATOM | 662 | CA | ALA | A | 101 | −48.154 | −12.500 | 36.911 | 1.00 | 52.60 | A |
| ATOM | 663 | CB | ALA | A | 101 | −47.522 | −13.538 | 35.983 | 1.00 | 51.25 | A |
| ATOM | 664 | C | ALA | A | 101 | −47.962 | −12.902 | 38.373 | 1.00 | 54.16 | A |
| ATOM | 665 | O | ALA | A | 101 | −46.842 | −12.899 | 38.894 | 1.00 | 55.16 | A |
| ATOM | 666 | N | ARG | A | 102 | −49.068 | −13.221 | 39.035 | 1.00 | 55.38 | A |
| ATOM | 667 | CA | ARG | A | 102 | −49.062 | −13.627 | 40.439 | 1.00 | 56.20 | A |
| ATOM | 668 | CB | ARG | A | 102 | −49.725 | −12.556 | 41.289 | 1.00 | 57.35 | A |
| ATOM | 669 | CG | ARG | A | 102 | −48.764 | −11.563 | 41.856 | 1.00 | 59.79 | A |
| ATOM | 670 | CD | ARG | A | 102 | −48.330 | −11.977 | 43.232 | 1.00 | 61.30 | A |
| ATOM | 671 | NE | ARG | A | 102 | −48.551 | −10.882 | 44.172 | 1.00 | 64.68 | A |
| ATOM | 672 | CZ | ARG | A | 102 | −48.168 | −10.900 | 45.443 | 1.00 | 66.13 | A |
| ATOM | 673 | NH1 | ARG | A | 102 | −47.536 | −11.969 | 45.936 | 1.00 | 65.97 | A |
| ATOM | 674 | NH2 | ARG | A | 102 | −48.414 | −9.846 | 46.213 | 1.00 | 63.95 | A |
| ATOM | 675 | C | ARG | A | 102 | −49.799 | −14.950 | 40.593 | 1.00 | 56.53 | A |
| ATOM | 676 | O | ARG | A | 102 | −51.000 | −15.051 | 40.327 | 1.00 | 53.61 | A |
| ATOM | 677 | N | ASN | A | 103 | −49.049 | −15.957 | 41.028 | 1.00 | 57.61 | A |
| ATOM | 678 | CA | ASN | A | 103 | −49.528 | −17.326 | 41.194 | 1.00 | 60.00 | A |
| ATOM | 679 | CB | ASN | A | 103 | −50.677 | −17.423 | 42.230 | 1.00 | 64.44 | A |
| ATOM | 680 | CG | ASN | A | 103 | −52.067 | −17.212 | 41.623 | 1.00 | 69.10 | A |
| ATOM | 681 | OD1 | ASN | A | 103 | −52.287 | −17.436 | 40.420 | 1.00 | 68.97 | A |
| ATOM | 682 | ND2 | ASN | A | 103 | −53.025 | −16.800 | 42.470 | 1.00 | 70.03 | A |
| ATOM | 683 | C | ASN | A | 103 | −49.945 | −17.932 | 39.844 | 1.00 | 58.15 | A |
| ATOM | 684 | O | ASN | A | 103 | −50.496 | −19.035 | 39.782 | 1.00 | 58.84 | A |
| ATOM | 685 | N | GLY | A | 104 | −49.683 | −17.207 | 38.763 | 1.00 | 54.94 | A |
| ATOM | 686 | CA | GLY | A | 104 | −50.005 | −17.726 | 37.453 | 1.00 | 52.69 | A |
| ATOM | 687 | C | GLY | A | 104 | −51.076 | −17.005 | 36.666 | 1.00 | 51.17 | A |
| ATOM | 688 | O | GLY | A | 104 | −51.675 | −17.589 | 35.773 | 1.00 | 51.25 | A |
| ATOM | 689 | N | THR | A | 105 | −51.334 | −15.748 | 36.993 | 1.00 | 50.00 | A |
| ATOM | 690 | CA | THR | A | 105 | −52.331 | −14.972 | 36.266 | 1.00 | 48.32 | A |
| ATOM | 691 | CB | THR | A | 105 | −53.773 | −15.316 | 36.735 | 1.00 | 48.67 | A |
| ATOM | 692 | OG1 | THR | A | 105 | −53.791 | −15.549 | 38.151 | 1.00 | 48.55 | A |
| ATOM | 693 | CG2 | THR | A | 105 | −54.288 | −16.549 | 36.006 | 1.00 | 47.14 | A |
| ATOM | 694 | C | THR | A | 105 | −52.050 | −13.473 | 36.391 | 1.00 | 47.21 | A |
| ATOM | 695 | O | THR | A | 105 | −51.542 | −12.996 | 37.404 | 1.00 | 46.07 | A |
| ATOM | 696 | N | PRO | A | 106 | −52.384 | −12.704 | 35.350 | 1.00 | 46.91 | A |
| ATOM | 697 | CD | PRO | A | 106 | −52.958 | −13.048 | 34.041 | 1.00 | 47.27 | A |
| ATOM | 698 | CA | PRO | A | 106 | −52.117 | −11.270 | 35.424 | 1.00 | 46.04 | A |
| ATOM | 699 | CB | PRO | A | 106 | −52.505 | −10.760 | 34.025 | 1.00 | 44.78 | A |
| ATOM | 700 | CG | PRO | A | 106 | −52.388 | −11.944 | 33.166 | 1.00 | 46.79 | A |
| ATOM | 701 | C | PRO | A | 106 | −52.844 | −10.505 | 36.503 | 1.00 | 45.06 | A |
| ATOM | 702 | O | PRO | A | 106 | −54.052 | −10.636 | 36.675 | 1.00 | 46.46 | A |
| ATOM | 703 | N | VAL | A | 107 | −52.087 | −9.699 | 37.228 | 1.00 | 43.99 | A |
| ATOM | 704 | CA | VAL | A | 107 | −52.654 | −8.829 | 38.237 | 1.00 | 43.11 | A |
| ATOM | 705 | CB | VAL | A | 107 | −52.187 | −9.213 | 39.650 | 1.00 | 42.35 | A |
| ATOM | 706 | CG1 | VAL | A | 107 | −50.712 | −8.926 | 39.842 | 1.00 | 41.38 | A |
| ATOM | 707 | CG2 | VAL | A | 107 | −53.000 | −8.452 | 40.638 | 1.00 | 45.83 | A |
| ATOM | 708 | C | VAL | A | 107 | −52.160 | −7.427 | 37.853 | 1.00 | 41.86 | A |
| ATOM | 709 | O | VAL | A | 107 | −50.998 | −7.262 | 37.464 | 1.00 | 41.94 | A |
| ATOM | 710 | N | LEU | A | 108 | −53.028 | −6.427 | 37.920 | 1.00 | 40.40 | A |
| ATOM | 711 | CA | LEU | A | 108 | −52.606 | −5.077 | 37.548 | 1.00 | 43.30 | A |
| ATOM | 712 | CB | LEU | A | 108 | −53.819 | −4.147 | 37.417 | 1.00 | 41.46 | A |
| ATOM | 713 | CG | LEU | A | 108 | −53.545 | −2.706 | 36.980 | 1.00 | 41.40 | A |
| ATOM | 714 | CD1 | LEU | A | 108 | −54.610 | −2.264 | 36.004 | 1.00 | 43.66 | A |
| ATOM | 715 | CD2 | LEU | A | 108 | −53.531 | −1.771 | 38.180 | 1.00 | 40.59 | A |
| ATOM | 716 | C | LEU | A | 108 | −51.586 | −4.478 | 38.524 | 1.00 | 45.12 | A |
| ATOM | 717 | O | LEU | A | 108 | −51.780 | −4.495 | 39.746 | 1.00 | 46.59 | A |
| ATOM | 718 | N | LEU | A | 109 | −50.497 | −3.957 | 37.967 | 1.00 | 47.06 | A |
| ATOM | 719 | CA | LEU | A | 109 | −49.420 | −3.348 | 38.736 | 1.00 | 49.55 | A |
| ATOM | 720 | CB | LEU | A | 109 | −48.190 | −3.185 | 37.845 | 1.00 | 50.11 | A |
| ATOM | 721 | CG | LEU | A | 109 | −46.879 | −2.742 | 38.487 | 1.00 | 53.63 | A |
| ATOM | 722 | CD1 | LEU | A | 109 | −46.273 | −3.897 | 39.275 | 1.00 | 55.57 | A |
| ATOM | 723 | CD2 | LEU | A | 109 | −45.910 | −2.314 | 37.408 | 1.00 | 55.97 | A |
| ATOM | 724 | C | LEU | A | 109 | −49.865 | −1.983 | 39.237 | 1.00 | 51.23 | A |
| ATOM | 725 | O | LEU | A | 109 | −50.667 | −1.323 | 38.601 | 1.00 | 51.44 | A |
| ATOM | 726 | N | PRO | A | 110 | −49.376 | −1.557 | 40.409 | 1.00 | 54.54 | A |
| ATOM | 727 | CD | PRO | A | 110 | −48.894 | −2.432 | 41.491 | 1.00 | 55.48 | A |
| ATOM | 728 | CA | PRO | A | 110 | −49.762 | −.239 | 40.945 | 1.00 | 56.82 | A |
| ATOM | 729 | CB | PRO | A | 110 | −49.327 | −.325 | 42.404 | 1.00 | 56.02 | A |
| ATOM | 730 | CG | PRO | A | 110 | −49.529 | −1.785 | 42.714 | 1.00 | 56.85 | A |
| ATOM | 731 | C | PRO | A | 110 | −49.095 | .929 | 40.202 | 1.00 | 58.36 | A |
| ATOM | 732 | O | PRO | A | 110 | −47.874 | .963 | 40.043 | 1.00 | 57.86 | A |
| ATOM | 733 | N | SER | A | 111 | −49.893 | 1.894 | 39.761 | 1.00 | 60.96 | A |
| ATOM | 734 | CA | SER | A | 111 | −49.343 | 3.025 | 39.015 | 1.00 | 64.65 | A |
| ATOM | 735 | CB | SER | A | 111 | −50.467 | 3.765 | 38.276 | 1.00 | 65.65 | A |
| ATOM | 736 | OG | SER | A | 111 | −51.474 | 4.188 | 39.173 | 1.00 | 68.16 | A |
| ATOM | 737 | C | SER | A | 111 | −48.503 | 4.031 | 39.808 | 1.00 | 65.64 | A |
| ATOM | 738 | O | SER | A | 111 | −47.613 | 4.663 | 39.240 | 1.00 | 65.69 | A |
| ATOM | 739 | N | ASN | A | 112 | −48.784 | 4.189 | 41.103 | 1.00 | 67.10 | A |
| ATOM | 740 | CA | ASN | A | 112 | −48.032 | 5.125 | 41.949 | 1.00 | 68.17 | A |
| ATOM | 741 | CB | ASN | A | 112 | −48.670 | 6.521 | 41.918 | 1.00 | 70.63 | A |
| ATOM | 742 | CG | ASN | A | 112 | −50.183 | 6.484 | 42.099 | 1.00 | 74.26 | A |
| ATOM | 743 | OD1 | ASN | A | 112 | −50.702 | 6.026 | 43.130 | 1.00 | 75.22 | A |
| ATOM | 744 | ND2 | ASN | A | 112 | −50.903 | 6.971 | 41.087 | 1.00 | 74.95 | A |
| ATOM | 745 | C | ASN | A | 112 | −47.886 | 4.672 | 43.401 | 1.00 | 67.77 | A |
| ATOM | 746 | O | ASN | A | 112 | −48.712 | 3.915 | 43.921 | 1.00 | 66.93 | A |
| ATOM | 747 | N | PRO | A | 113 | −46.825 | 5.147 | 44.076 | 1.00 | 67.74 | A |
| ATOM | 748 | CD | PRO | A | 113 | −45.854 | 6.131 | 43.564 | 1.00 | 67.52 | A |
| ATOM | 749 | CA | PRO | A | 113 | −46.517 | 4.824 | 45.466 | 1.00 | 67.30 | A |
| ATOM | 750 | CB | PRO | A | 113 | −45.599 | 5.964 | 45.872 | 1.00 | 67.22 | A |
| ATOM | 751 | CG | PRO | A | 113 | −44.789 | 6.133 | 44.647 | 1.00 | 66.04 | A |
| ATOM | 752 | C | PRO | A | 113 | −47.741 | 4.705 | 46.354 | 1.00 | 67.20 | A |
| ATOM | 753 | O | PRO | A | 113 | −47.890 | 3.717 | 47.063 | 1.00 | 67.04 | A |
| ATOM | 754 | N | ILE | A | 114 | −48.620 | 5.701 | 46.317 | 1.00 | 66.96 | A |
| ATOM | 755 | CA | ILE | A | 114 | −49.822 | 5.654 | 47.144 | 1.00 | 66.77 | A |
| ATOM | 756 | CB | ILE | A | 114 | −50.812 | 6.801 | 46.801 | 1.00 | 66.67 | A |
| ATOM | 757 | CG2 | ILE | A | 114 | −51.994 | 6.796 | 47.779 | 1.00 | 63.09 | A |
| ATOM | 758 | CG1 | ILE | A | 114 | −50.092 | 8.144 | 46.875 | 1.00 | 66.62 | A |
| ATOM | 759 | CD1 | ILE | A | 114 | −49.511 | 8.433 | 48.237 | 1.00 | 69.34 | A |
| ATOM | 760 | C | ILE | A | 114 | −50.555 | 4.319 | 46.988 | 1.00 | 67.47 | A |
| ATOM | 761 | O | ILE | A | 114 | −50.710 | 3.572 | 47.963 | 1.00 | 67.56 | A |
| ATOM | 762 | N | ASP | A | 115 | −50.995 | 4.007 | 45.769 | 1.00 | 66.98 | A |
| ATOM | 763 | CA | ASP | A | 115 | −51.723 | 2.763 | 45.548 | 1.00 | 67.00 | A |
| ATOM | 764 | CB | ASP | A | 115 | −52.038 | 2.589 | 44.062 | 1.00 | 69.90 | A |
| ATOM | 765 | CG | ASP | A | 115 | −53.259 | 3.405 | 43.629 | 1.00 | 75.06 | A |
| ATOM | 766 | OD1 | ASP | A | 115 | −54.331 | 3.270 | 44.279 | 1.00 | 74.86 | A |
| ATOM | 767 | OD2 | ASP | A | 115 | −53.150 | 4.179 | 42.644 | 1.00 | 77.58 | A |
| ATOM | 768 | C | ASP | A | 115 | −51.011 | 1.533 | 46.105 | 1.00 | 65.05 | A |
| ATOM | 769 | O | ASP | A | 115 | −51.636 | .509 | 46.357 | 1.00 | 62.88 | A |
| ATOM | 770 | N | LEU | A | 116 | −49.706 | 1.650 | 46.315 | 1.00 | 64.21 | A |
| ATOM | 771 | CA | LEU | A | 116 | −48.914 | .561 | 46.863 | 1.00 | 64.05 | A |
| ATOM | 772 | CB | LEU | A | 116 | −47.438 | .952 | 46.841 | 1.00 | 63.98 | A |
| ATOM | 773 | CG | LEU | A | 116 | −46.375 | −.101 | 46.530 | 1.00 | 63.95 | A |
| ATOM | 774 | CD1 | LEU | A | 116 | −46.909 | −1.107 | 45.519 | 1.00 | 63.86 | A |
| ATOM | 775 | CD2 | LEU | A | 116 | −45.132 | .603 | 45.984 | 1.00 | 61.90 | A |
| ATOM | 776 | C | LEU | A | 116 | −49.368 | .270 | 48.299 | 1.00 | 65.15 | A |
| ATOM | 777 | O | LEU | A | 116 | −49.545 | −.891 | 48.680 | 1.00 | 67.84 | A |
| ATOM | 778 | N | ILE | A | 117 | −49.568 | 1.321 | 49.091 | 1.00 | 64.34 | A |
| ATOM | 779 | CA | ILE | A | 117 | −50.016 | 1.172 | 50.475 | 1.00 | 62.95 | A |
| ATOM | 780 | CB | ILE | A | 117 | −49.561 | 2.383 | 51.356 | 1.00 | 63.93 | A |
| ATOM | 781 | CG2 | ILE | A | 117 | −50.215 | 2.325 | 52.730 | 1.00 | 63.79 | A |
| ATOM | 782 | CG1 | ILE | A | 117 | −48.036 | 2.385 | 51.525 | 1.00 | 63.92 | A |
| ATOM | 783 | CD1 | ILE | A | 117 | −47.474 | 1.170 | 52.252 | 1.00 | 62.37 | A |
| ATOM | 784 | C | ILE | A | 117 | −51.544 | 1.066 | 50.531 | 1.00 | 62.19 | A |
| ATOM | 785 | O | ILE | A | 117 | −52.102 | .479 | 51.459 | 1.00 | 61.94 | A |
| ATOM | 786 | N | ALA | A | 118 | −52.213 | 1.624 | 49.524 | 1.00 | 61.12 | A |
| ATOM | 787 | CA | ALA | A | 118 | −53.675 | 1.616 | 49.461 | 1.00 | 59.46 | A |
| ATOM | 788 | CB | ALA | A | 118 | −54.169 | 2.855 | 48.697 | 1.00 | 57.70 | A |
| ATOM | 789 | C | ALA | A | 118 | −54.298 | .365 | 48.847 | 1.00 | 58.52 | A |
| ATOM | 790 | O | ALA | A | 118 | −55.469 | .091 | 49.076 | 1.00 | 58.68 | A |
| ATOM | 791 | N | SER | A | 119 | −53.532 | −.390 | 48.063 | 1.00 | 58.06 | A |
| ATOM | 792 | CA | SER | A | 119 | −54.070 | −1.584 | 47.410 | 1.00 | 56.77 | A |
| ATOM | 793 | CB | SER | A | 119 | −53.455 | −1.769 | 46.019 | 1.00 | 57.56 | A |
| ATOM | 794 | OG | SER | A | 119 | −52.129 | −2.266 | 46.101 | 1.00 | 58.80 | A |
| ATOM | 795 | C | SER | A | 119 | −53.790 | −2.817 | 48.227 | 1.00 | 55.63 | A |
| ATOM | 796 | O | SER | A | 119 | −53.363 | −2.721 | 49.372 | 1.00 | 55.66 | A |
| ATOM | 797 | N | ASN | A | 120 | −54.039 | −3.981 | 47.640 | 1.00 | 54.23 | A |
| ATOM | 798 | CA | ASN | A | 120 | −53.778 | −5.219 | 48.345 | 1.00 | 53.91 | A |
| ATOM | 799 | CB | ASN | A | 120 | −55.043 | −6.066 | 48.437 | 1.00 | 55.77 | A |
| ATOM | 800 | CG | ASN | A | 120 | −55.584 | −6.134 | 49.863 | 1.00 | 58.33 | A |
| ATOM | 801 | OD1 | ASN | A | 120 | −55.825 | −5.098 | 50.495 | 1.00 | 59.47 | A |
| ATOM | 802 | ND2 | ASN | A | 120 | −55.768 | −7.351 | 50.378 | 1.00 | 57.88 | A |
| ATOM | 803 | C | ASN | A | 120 | −52.664 | −5.969 | 47.650 | 1.00 | 52.61 | A |
| ATOM | 804 | O | ASN | A | 120 | −52.345 | −7.109 | 47.984 | 1.00 | 52.43 | A |
| ATOM | 805 | N | PHE | A | 121 | −52.053 | −5.302 | 46.685 | 1.00 | 50.82 | A |
| ATOM | 806 | CA | PHE | A | 121 | −50.961 | −5.902 | 45.957 | 1.00 | 50.20 | A |
| ATOM | 807 | CB | PHE | A | 121 | −50.425 | −4.923 | 44.920 | 1.00 | 49.52 | A |
| ATOM | 808 | CG | PHE | A | 121 | −49.243 | −5.437 | 44.174 | 1.00 | 47.49 | A |
| ATOM | 809 | CD1 | PHE | A | 121 | −49.409 | −6.218 | 43.048 | 1.00 | 47.00 | A |
| ATOM | 810 | CD2 | PHE | A | 121 | −47.957 | −5.152 | 44.612 | 1.00 | 47.67 | A |
| ATOM | 811 | CE1 | PHE | A | 121 | −48.319 | −6.706 | 42.363 | 1.00 | 47.69 | A |
| ATOM | 812 | CE2 | PHE | A | 121 | −46.859 | −5.635 | 43.934 | 1.00 | 48.34 | A |
| ATOM | 813 | CZ | PHE | A | 121 | −47.043 | −6.416 | 42.801 | 1.00 | 48.01 | A |
| ATOM | 814 | C | PHE | A | 121 | −49.829 | −6.331 | 46.901 | 1.00 | 50.18 | A |
| ATOM | 815 | O | PHE | A | 121 | −49.049 | −7.228 | 46.575 | 1.00 | 52.48 | A |
| ATOM | 816 | N | LEU | A | 122 | −49.728 | −5.687 | 48.058 | 1.00 | 48.30 | A |
| ATOM | 817 | CA | LEU | A | 122 | −48.696 | −6.038 | 49.034 | 1.00 | 46.47 | A |
| ATOM | 818 | CB | LEU | A | 122 | −47.799 | −4.838 | 49.353 | 1.00 | 45.61 | A |
| ATOM | 819 | CG | LEU | A | 122 | −46.723 | −4.445 | 48.351 | 1.00 | 45.09 | A |
| ATOM | 820 | CD1 | LEU | A | 122 | −46.123 | −3.122 | 48.773 | 1.00 | 43.71 | A |
| ATOM | 821 | CD2 | LEU | A | 122 | −45.671 | −5.530 | 48.281 | 1.00 | 43.44 | A |
| ATOM | 822 | C | LEU | A | 122 | −49.335 | −6.501 | 50.324 | 1.00 | 45.57 | A |
| ATOM | 823 | O | LEU | A | 122 | −50.440 | −6.082 | 50.660 | 1.00 | 45.73 | A |
| ATOM | 824 | N | SER | A | 123 | −48.625 | −7.358 | 51.046 | 1.00 | 45.54 | A |
| ATOM | 825 | CA | SER | A | 123 | −49.084 | −7.882 | 52.331 | 1.00 | 45.57 | A |
| ATOM | 826 | CB | SER | A | 123 | −48.175 | −9.011 | 52.762 | 1.00 | 45.50 | A |
| ATOM | 827 | OG | SER | A | 123 | −46.861 | −8.518 | 52.955 | 1.00 | 45.24 | A |
| ATOM | 828 | C | SER | A | 123 | −49.055 | −6.810 | 53.425 | 1.00 | 46.76 | A |
| ATOM | 829 | O | SER | A | 123 | −48.406 | −5.767 | 53.282 | 1.00 | 46.73 | A |
| ATOM | 830 | N | THR | A | 124 | −49.741 | −7.077 | 54.532 | 1.00 | 47.26 | A |
| ATOM | 831 | CA | THR | A | 124 | −49.766 | −6.128 | 55.649 | 1.00 | 46.02 | A |
| ATOM | 832 | CB | THR | A | 124 | −50.763 | −6.594 | 56.754 | 1.00 | 47.02 | A |
| ATOM | 833 | OG1 | THR | A | 124 | −52.108 | −6.470 | 56.259 | 1.00 | 48.14 | A |
| ATOM | 834 | CG2 | THR | A | 124 | −50.621 | −5.756 | 58.012 | 1.00 | 45.56 | A |
| ATOM | 835 | C | THR | A | 124 | −48.362 | −5.915 | 56.234 | 1.00 | 42.78 | A |
| ATOM | 836 | O | THR | A | 124 | −48.016 | −4.810 | 56.636 | 1.00 | 41.81 | A |
| ATOM | 837 | N | GLY | A | 125 | −47.549 | −6.960 | 56.238 | 1.00 | 41.37 | A |
| ATOM | 838 | CA | GLY | A | 125 | −46.201 | −6.846 | 56.759 | 1.00 | 42.12 | A |
| ATOM | 839 | C | GLY | A | 125 | −45.258 | −6.089 | 55.839 | 1.00 | 44.56 | A |
| ATOM | 840 | O | GLY | A | 125 | −44.240 | −5.547 | 56.282 | 1.00 | 44.77 | A |
| ATOM | 841 | N | SER | A | 126 | −45.583 | −6.037 | 54.551 | 1.00 | 44.63 | A |
| ATOM | 842 | CA | SER | A | 126 | −44.727 | −5.328 | 53.611 | 1.00 | 45.23 | A |
| ATOM | 843 | CB | SER | A | 126 | −44.965 | −5.844 | 52.195 | 1.00 | 46.04 | A |
| ATOM | 844 | OG | SER | A | 126 | −44.520 | −7.178 | 52.075 | 1.00 | 48.89 | A |
| ATOM | 845 | C | SER | A | 126 | −44.951 | −3.817 | 53.653 | 1.00 | 45.68 | A |
| ATOM | 846 | O | SER | A | 126 | −44.008 | −3.034 | 53.503 | 1.00 | 45.31 | A |
| ATOM | 847 | N | LYS | A | 127 | −46.204 | −3.419 | 53.851 | 1.00 | 45.01 | A |
| ATOM | 848 | CA | LYS | A | 127 | −46.568 | −2.012 | 53.908 | 1.00 | 45.45 | A |
| ATOM | 849 | CB | LYS | A | 127 | −48.082 | −1.862 | 53.967 | 1.00 | 42.49 | A |
| ATOM | 850 | CG | LYS | A | 127 | −48.820 | −2.640 | 52.930 | 1.00 | 40.91 | A |
| ATOM | 851 | CD | LYS | A | 127 | −50.261 | −2.223 | 52.905 | 1.00 | 39.06 | A |
| ATOM | 852 | CE | LYS | A | 127 | −51.021 | −3.008 | 51.893 | 1.00 | 37.30 | A |
| ATOM | 853 | NZ | LYS | A | 127 | −52.426 | −2.584 | 51.936 | 1.00 | 38.91 | A |
| ATOM | 854 | C | LYS | A | 127 | −45.976 | −1.370 | 55.152 | 1.00 | 48.04 | A |
| ATOM | 855 | O | LYS | A | 127 | −45.446 | −.252 | 55.123 | 1.00 | 48.64 | A |
| ATOM | 856 | N | LEU | A | 128 | −46.102 | −2.080 | 56.262 | 1.00 | 49.27 | A |
| ATOM | 857 | CA | LEU | A | 128 | −45.580 | −1.585 | 57.515 | 1.00 | 50.44 | A |
| ATOM | 858 | CB | LEU | A | 128 | −46.002 | −2.509 | 58.665 | 1.00 | 50.90 | A |
| ATOM | 859 | CG | LEU | A | 128 | −47.524 | −2.698 | 58.852 | 1.00 | 50.99 | A |
| ATOM | 860 | CD1 | LEU | A | 128 | −47.769 | −3.610 | 60.045 | 1.00 | 48.15 | A |
| ATOM | 861 | CD2 | LEU | A | 128 | −48.223 | −1.356 | 59.054 | 1.00 | 51.07 | A |
| ATOM | 862 | C | LEU | A | 128 | −44.065 | −1.500 | 57.410 | 1.00 | 50.47 | A |
| ATOM | 863 | O | LEU | A | 128 | −43.442 | −.572 | 57.919 | 1.00 | 52.21 | A |
| ATOM | 864 | N | GLN | A | 129 | −43.464 | −2.461 | 56.729 | 1.00 | 49.24 | A |
| ATOM | 865 | CA | GLN | A | 129 | −42.026 | −2.436 | 56.584 | 1.00 | 48.31 | A |
| ATOM | 866 | CB | GLN | A | 129 | −41.533 | −3.785 | 56.072 | 1.00 | 47.95 | A |
| ATOM | 867 | CG | GLN | A | 129 | −40.094 | −3.763 | 55.603 | 1.00 | 49.27 | A |
| ATOM | 868 | CD | GLN | A | 129 | −39.484 | −5.151 | 55.481 | 1.00 | 51.20 | A |
| ATOM | 869 | OE1 | GLN | A | 129 | −40.186 | −6.146 | 55.260 | 1.00 | 53.52 | A |
| ATOM | 870 | NE2 | GLN | A | 129 | −38.162 | −5.225 | 55.612 | 1.00 | 50.93 | A |
| ATOM | 871 | C | GLN | A | 129 | −41.561 | −1.307 | 55.668 | 1.00 | 48.52 | A |
| ATOM | 872 | O | GLN | A | 129 | −40.427 | −.848 | 55.780 | 1.00 | 48.36 | A |
| ATOM | 873 | N | MSE | A | 130 | −42.435 | −.869 | 54.764 | 1.00 | 49.72 | A |
| ATOM | 874 | CA | MSE | A | 130 | −42.132 | .214 | 53.829 | 1.00 | 50.95 | A |
| ATOM | 875 | CB | MSE | A | 130 | −43.148 | .227 | 52.689 | 1.00 | 56.03 | A |
| ATOM | 876 | CG | MSE | A | 130 | −42.826 | 1.121 | 51.494 | 1.00 | 60.37 | A |
| ATOM | 877 | SE | MSE | A | 130 | −43.992 | .686 | 49.964 | 1.00 | 65.83 | A |
| ATOM | 878 | CE | MSE | A | 130 | −44.933 | 2.361 | 49.865 | 1.00 | 65.93 | A |
| ATOM | 879 | C | MSE | A | 130 | −42.255 | 1.500 | 54.609 | 1.00 | 49.80 | A |
| ATOM | 880 | O | MSE | A | 130 | −41.282 | 2.218 | 54.779 | 1.00 | 50.33 | A |
| ATOM | 881 | N | LEU | A | 131 | −43.463 | 1.771 | 55.094 | 1.00 | 48.32 | A |
| ATOM | 882 | CA | LEU | A | 131 | −43.745 | 2.969 | 55.875 | 1.00 | 47.88 | A |
| ATOM | 883 | CB | LEU | A | 131 | −45.147 | 2.888 | 56.466 | 1.00 | 46.29 | A |
| ATOM | 884 | CG | LEU | A | 131 | −46.248 | 3.014 | 55.421 | 1.00 | 47.15 | A |
| ATOM | 885 | CD1 | LEU | A | 131 | −47.569 | 2.708 | 56.061 | 1.00 | 45.61 | A |
| ATOM | 886 | CD2 | LEU | A | 131 | −46.243 | 4.428 | 54.813 | 1.00 | 49.93 | A |
| ATOM | 887 | C | LEU | A | 131 | −42.741 | 3.228 | 56.993 | 1.00 | 47.90 | A |
| ATOM | 888 | O | LEU | A | 131 | −42.377 | 4.379 | 57.260 | 1.00 | 49.16 | A |
| ATOM | 889 | N | LEU | A | 132 | −42.286 | 2.170 | 57.647 | 1.00 | 45.70 | A |
| ATOM | 890 | CA | LEU | A | 132 | −41.319 | 2.341 | 58.719 | 1.00 | 45.68 | A |
| ATOM | 891 | CB | LEU | A | 132 | −41.538 | 1.277 | 59.815 | 1.00 | 41.01 | A |
| ATOM | 892 | CG | LEU | A | 132 | −42.895 | 1.334 | 60.534 | 1.00 | 36.94 | A |
| ATOM | 893 | CD1 | LEU | A | 132 | −42.995 | .217 | 61.532 | 1.00 | 29.80 | A |
| ATOM | 894 | CD2 | LEU | A | 132 | −43.073 | 2.679 | 61.200 | 1.00 | 34.57 | A |
| ATOM | 895 | C | LEU | A | 132 | −39.879 | 2.296 | 58.197 | 1.00 | 46.64 | A |
| ATOM | 896 | O | LEU | A | 132 | −38.920 | 2.327 | 58.973 | 1.00 | 47.47 | A |
| ATOM | 897 | N | GLU | A | 133 | −39.724 | 2.243 | 56.881 | 1.00 | 47.42 | A |
| ATOM | 898 | CA | GLU | A | 133 | −38.390 | 2.189 | 56.289 | 1.00 | 51.09 | A |
| ATOM | 899 | CB | GLU | A | 133 | −38.513 | 2.027 | 54.769 | 1.00 | 50.30 | A |
| ATOM | 900 | CG | GLU | A | 133 | −37.257 | 1.577 | 54.043 | 1.00 | 51.37 | A |
| ATOM | 901 | CD | GLU | A | 133 | −36.322 | 2.724 | 53.713 | 1.00 | 53.45 | A |
| ATOM | 902 | OE1 | GLU | A | 133 | −36.815 | 3.808 | 53.368 | 1.00 | 53.02 | A |
| ATOM | 903 | OE2 | GLU | A | 133 | −35.089 | 2.545 | 53.775 | 1.00 | 56.62 | A |
| ATOM | 904 | C | GLU | A | 133 | −37.552 | 3.436 | 56.650 | 1.00 | 53.23 | A |
| ATOM | 905 | O | GLU | A | 133 | −36.321 | 3.362 | 56.802 | 1.00 | 51.20 | A |
| ATOM | 906 | N | PRO | A | 134 | −38.213 | 4.602 | 56.790 | 1.00 | 55.05 | A |
| ATOM | 907 | CD | PRO | A | 134 | −39.590 | 4.945 | 56.391 | 1.00 | 53.81 | A |
| ATOM | 908 | CA | PRO | A | 134 | −37.460 | 5.815 | 57.137 | 1.00 | 56.26 | A |
| ATOM | 909 | CB | PRO | A | 134 | −38.519 | 6.920 | 57.036 | 1.00 | 54.77 | A |
| ATOM | 910 | CG | PRO | A | 134 | −39.452 | 6.400 | 56.005 | 1.00 | 53.92 | A |
| ATOM | 911 | C | PRO | A | 134 | −36.841 | 5.710 | 58.537 | 1.00 | 57.71 | A |
| ATOM | 912 | O | PRO | A | 134 | −35.787 | 6.274 | 58.800 | 1.00 | 57.60 | A |
| ATOM | 913 | N | ILE | A | 135 | −37.498 | 4.979 | 59.430 | 1.00 | 59.52 | A |
| ATOM | 914 | CA | ILE | A | 135 | −36.988 | 4.803 | 60.784 | 1.00 | 61.54 | A |
| ATOM | 915 | CB | ILE | A | 135 | −38.142 | 4.696 | 61.797 | 1.00 | 60.96 | A |
| ATOM | 916 | CG2 | ILE | A | 135 | −37.689 | 3.971 | 63.061 | 1.00 | 60.76 | A |
| ATOM | 917 | CG1 | ILE | A | 135 | −38.614 | 6.091 | 62.166 | 1.00 | 60.86 | A |
| ATOM | 918 | CD1 | ILE | A | 135 | −37.604 | 6.857 | 62.971 | 1.00 | 60.83 | A |
| ATOM | 919 | C | ILE | A | 135 | −36.081 | 3.579 | 60.940 | 1.00 | 63.54 | A |
| ATOM | 920 | O | ILE | A | 135 | −35.025 | 3.665 | 61.578 | 1.00 | 63.83 | A |
| ATOM | 921 | N | LEU | A | 136 | −36.492 | 2.445 | 60.372 | 1.00 | 64.48 | A |
| ATOM | 922 | CA | LEU | A | 136 | −35.707 | 1.214 | 60.476 | 1.00 | 66.07 | A |
| ATOM | 923 | CB | LEU | A | 136 | −36.545 | .030 | 60.019 | 1.00 | 63.82 | A |
| ATOM | 924 | CG | LEU | A | 136 | −37.765 | −.265 | 60.875 | 1.00 | 64.25 | A |
| ATOM | 925 | CD1 | LEU | A | 136 | −38.863 | −.918 | 60.043 | 1.00 | 63.75 | A |
| ATOM | 926 | CD2 | LEU | A | 136 | −37.336 | −1.152 | 62.024 | 1.00 | 63.01 | A |
| ATOM | 927 | C | LEU | A | 136 | −34.414 | 1.265 | 59.655 | 1.00 | 68.47 | A |
| ATOM | 928 | O | LEU | A | 136 | −33.527 | .415 | 59.807 | 1.00 | 68.56 | A |
| ATOM | 929 | N | TRP | A | 137 | −34.303 | 2.271 | 58.794 | 1.00 | 69.61 | A |
| ATOM | 930 | CA | TRP | A | 137 | −33.137 | 2.404 | 57.942 | 1.00 | 70.74 | A |
| ATOM | 931 | CB | TRP | A | 137 | −33.311 | 1.519 | 56.696 | 1.00 | 72.74 | A |
| ATOM | 932 | CG | TRP | A | 137 | −32.304 | .413 | 56.596 | 1.00 | 74.54 | A |
| ATOM | 933 | CD2 | TRP | A | 137 | −32.506 | −.969 | 56.915 | 1.00 | 75.25 | A |
| ATOM | 934 | CE2 | TRP | A | 137 | −31.250 | −1.615 | 56.791 | 1.00 | 75.73 | A |
| ATOM | 935 | CE3 | TRP | A | 137 | −33.619 | −1.724 | 57.302 | 1.00 | 76.55 | A |
| ATOM | 936 | CD1 | TRP | A | 137 | −30.981 | .543 | 56.288 | 1.00 | 75.56 | A |
| ATOM | 937 | NE1 | TRP | A | 137 | −30.340 | −.668 | 56.405 | 1.00 | 75.59 | A |
| ATOM | 938 | CZ2 | TRP | A | 137 | −31.074 | −2.984 | 57.043 | 1.00 | 75.36 | A |
| ATOM | 939 | CZ3 | TRP | A | 137 | −33.444 | −3.095 | 57.556 | 1.00 | 79.03 | A |
| ATOM | 940 | CH2 | TRP | A | 137 | −32.175 | −3.705 | 57.424 | 1.00 | 76.82 | A |
| ATOM | 941 | C | TRP | A | 137 | −32.970 | 3.860 | 57.534 | 1.00 | 72.10 | A |
| ATOM | 942 | O | TRP | A | 137 | −32.199 | 4.606 | 58.141 | 1.00 | 72.80 | A |
| ATOM | 943 | N | SER | A | 148 | −20.641 | −2.481 | 49.145 | 1.00 | 85.83 | A |
| ATOM | 944 | CA | SER | A | 148 | −19.421 | −2.930 | 48.473 | 1.00 | 86.02 | A |
| ATOM | 945 | CB | SER | A | 148 | −18.554 | −3.782 | 49.423 | 1.00 | 85.74 | A |
| ATOM | 946 | OG | SER | A | 148 | −18.114 | −3.048 | 50.551 | 1.00 | 85.29 | A |
| ATOM | 947 | C | SER | A | 148 | −19.729 | −3.756 | 47.219 | 1.00 | 84.95 | A |
| ATOM | 948 | O | SER | A | 148 | −19.353 | −3.380 | 46.097 | 1.00 | 85.58 | A |
| ATOM | 949 | N | HIS | A | 149 | −20.405 | −4.884 | 47.426 | 1.00 | 81.97 | A |
| ATOM | 950 | CA | HIS | A | 149 | −20.753 | −5.798 | 46.344 | 1.00 | 79.16 | A |
| ATOM | 951 | CB | HIS | A | 149 | −19.860 | −7.045 | 46.406 | 1.00 | 81.07 | A |
| ATOM | 952 | CG | HIS | A | 149 | −18.428 | −6.794 | 46.045 | 1.00 | 82.42 | A |
| ATOM | 953 | CD2 | HIS | A | 149 | −17.821 | −5.720 | 45.483 | 1.00 | 82.85 | A |
| ATOM | 954 | ND1 | HIS | A | 149 | −17.436 | −7.729 | 46.253 | 1.00 | 82.60 | A |
| ATOM | 955 | CE1 | HIS | A | 149 | −16.281 | −7.242 | 45.837 | 1.00 | 82.54 | A |
| ATOM | 956 | NE2 | HIS | A | 149 | −16.486 | −6.025 | 45.366 | 1.00 | 82.75 | A |
| ATOM | 957 | C | HIS | A | 149 | −22.210 | −6.238 | 46.424 | 1.00 | 75.82 | A |
| ATOM | 958 | O | HIS | A | 149 | −22.630 | −7.152 | 45.711 | 1.00 | 76.53 | A |
| ATOM | 959 | N | GLU | A | 150 | −22.977 | −5.590 | 47.291 | 1.00 | 70.39 | A |
| ATOM | 960 | CA | GLU | A | 150 | −24.379 | −5.938 | 47.473 | 1.00 | 65.79 | A |
| ATOM | 961 | CB | GLU | A | 150 | −25.053 | −4.871 | 48.341 | 1.00 | 66.56 | A |
| ATOM | 962 | CG | GLU | A | 150 | −26.481 | −5.199 | 48.699 | 1.00 | 68.55 | A |
| ATOM | 963 | CD | GLU | A | 150 | −26.856 | −4.776 | 50.105 | 1.00 | 68.77 | A |
| ATOM | 964 | OE1 | GLU | A | 150 | −26.897 | −3.551 | 50.366 | 1.00 | 67.77 | A |
| ATOM | 965 | OE2 | GLU | A | 150 | −27.107 | −5.681 | 50.939 | 1.00 | 67.52 | A |
| ATOM | 966 | C | GLU | A | 150 | −25.158 | −6.134 | 46.163 | 1.00 | 61.98 | A |
| ATOM | 967 | O | GLU | A | 150 | −25.034 | −5.349 | 45.229 | 1.00 | 60.61 | A |
| ATOM | 968 | N | SER | A | 151 | −25.948 | −7.201 | 46.094 | 1.00 | 58.51 | A |
| ATOM | 969 | CA | SER | A | 151 | −26.746 | −7.475 | 44.910 | 1.00 | 53.97 | A |
| ATOM | 970 | CB | SER | A | 151 | −27.123 | −8.948 | 44.835 | 1.00 | 52.68 | A |
| ATOM | 971 | OG | SER | A | 151 | −28.097 | −9.271 | 45.801 | 1.00 | 46.86 | A |
| ATOM | 972 | C | SER | A | 151 | −28.016 | −6.667 | 45.004 | 1.00 | 53.21 | A |
| ATOM | 973 | O | SER | A | 151 | −28.421 | −6.283 | 46.088 | 1.00 | 54.15 | A |
| ATOM | 974 | N | VAL | A | 152 | −28.647 | −6.421 | 43.863 | 1.00 | 52.40 | A |
| ATOM | 975 | CA | VAL | A | 152 | −29.897 | −5.667 | 43.813 | 1.00 | 50.46 | A |
| ATOM | 976 | CB | VAL | A | 152 | −30.550 | −5.759 | 42.419 | 1.00 | 48.89 | A |
| ATOM | 977 | CG1 | VAL | A | 152 | −31.910 | −5.123 | 42.450 | 1.00 | 49.82 | A |
| ATOM | 978 | CG2 | VAL | A | 152 | −29.697 | −5.065 | 41.395 | 1.00 | 48.31 | A |
| ATOM | 979 | C | VAL | A | 152 | −30.850 | −6.285 | 44.816 | 1.00 | 49.83 | A |
| ATOM | 980 | O | VAL | A | 152 | −31.467 | −5.593 | 45.621 | 1.00 | 51.08 | A |
| ATOM | 981 | N | SER | A | 153 | −30.933 | −7.608 | 44.744 | 1.00 | 49.25 | A |
| ATOM | 982 | CA | SER | A | 153 | −31.774 | −8.451 | 45.585 | 1.00 | 48.36 | A |
| ATOM | 983 | CB | SER | A | 153 | −31.528 | −9.902 | 45.175 | 1.00 | 47.65 | A |
| ATOM | 984 | OG | SER | A | 153 | −32.384 | −10.796 | 45.837 | 1.00 | 52.43 | A |
| ATOM | 985 | C | SER | A | 153 | −31.530 | −8.267 | 47.091 | 1.00 | 48.73 | A |
| ATOM | 986 | O | SER | A | 153 | −32.470 | −8.080 | 47.862 | 1.00 | 48.63 | A |
| ATOM | 987 | N | GLY | A | 154 | −30.269 | −8.304 | 47.508 | 1.00 | 48.72 | A |
| ATOM | 988 | CA | GLY | A | 154 | −29.953 | −8.153 | 48.918 | 1.00 | 49.04 | A |
| ATOM | 989 | C | GLY | A | 154 | −30.389 | −6.821 | 49.475 | 1.00 | 48.76 | A |
| ATOM | 990 | O | GLY | A | 154 | −31.061 | −6.732 | 50.511 | 1.00 | 49.15 | A |
| ATOM | 991 | N | PHE | A | 155 | −29.990 | −5.774 | 48.775 | 1.00 | 47.63 | A |
| ATOM | 992 | CA | PHE | A | 155 | −30.330 | −4.421 | 49.163 | 1.00 | 48.87 | A |
| ATOM | 993 | CB | PHE | A | 155 | −29.930 | −3.474 | 48.047 | 1.00 | 50.05 | A |
| ATOM | 994 | CG | PHE | A | 155 | −30.282 | −2.038 | 48.288 | 1.00 | 49.78 | A |
| ATOM | 995 | CD1 | PHE | A | 155 | −29.285 | −1.105 | 48.567 | 1.00 | 49.58 | A |
| ATOM | 996 | CD2 | PHE | A | 155 | −31.583 | −1.587 | 48.090 | 1.00 | 50.82 | A |
| ATOM | 997 | CE1 | PHE | A | 155 | −29.573 | .257 | 48.628 | 1.00 | 48.40 | A |
| ATOM | 998 | CE2 | PHE | A | 155 | −31.878 | −.235 | 48.148 | 1.00 | 51.24 | A |
| ATOM | 999 | CZ | PHE | A | 155 | −30.861 | .691 | 48.415 | 1.00 | 50.84 | A |
| ATOM | 1000 | C | PHE | A | 155 | −31.822 | −4.354 | 49.392 | 1.00 | 49.38 | A |
| ATOM | 1001 | O | PHE | A | 155 | −32.270 | −4.324 | 50.539 | 1.00 | 51.32 | A |
| ATOM | 1002 | N | PHE | A | 156 | −32.593 | −4.358 | 48.306 | 1.00 | 47.60 | A |
| ATOM | 1003 | CA | PHE | A | 156 | −34.049 | −4.287 | 48.414 | 1.00 | 46.81 | A |
| ATOM | 1004 | CB | PHE | A | 156 | −34.687 | −4.508 | 47.047 | 1.00 | 44.24 | A |
| ATOM | 1005 | CG | PHE | A | 156 | −34.529 | −3.339 | 46.129 | 1.00 | 42.60 | A |
| ATOM | 1006 | CD1 | PHE | A | 156 | −33.556 | −3.338 | 45.129 | 1.00 | 43.05 | A |
| ATOM | 1007 | CD2 | PHE | A | 156 | −35.309 | −2.206 | 46.301 | 1.00 | 40.20 | A |
| ATOM | 1008 | CE1 | PHE | A | 156 | −33.362 | −2.217 | 44.314 | 1.00 | 40.55 | A |
| ATOM | 1009 | CE2 | PHE | A | 156 | −35.124 | −1.091 | 45.499 | 1.00 | 39.95 | A |
| ATOM | 1010 | CZ | PHE | A | 156 | −34.143 | −1.096 | 44.499 | 1.00 | 39.87 | A |
| ATOM | 1011 | C | PHE | A | 156 | −34.659 | −5.233 | 49.451 | 1.00 | 48.00 | A |
| ATOM | 1012 | O | PHE | A | 156 | −35.618 | −4.869 | 50.142 | 1.00 | 45.71 | A |
| ATOM | 1013 | N | GLN | A | 157 | −34.105 | −6.436 | 49.561 | 1.00 | 49.19 | A |
| ATOM | 1014 | CA | GLN | A | 157 | −34.588 | −7.390 | 50.534 | 1.00 | 52.74 | A |
| ATOM | 1015 | CB | GLN | A | 157 | −33.711 | −8.634 | 50.536 | 1.00 | 54.48 | A |
| ATOM | 1016 | CG | GLN | A | 157 | −33.923 | −9.560 | 51.727 | 1.00 | 57.92 | A |
| ATOM | 1017 | CD | GLN | A | 157 | −35.162 | −10.404 | 51.590 | 1.00 | 63.23 | A |
| ATOM | 1018 | OE1 | GLN | A | 157 | −35.085 | −11.616 | 51.340 | 1.00 | 65.14 | A |
| ATOM | 1019 | NE2 | GLN | A | 157 | −36.327 | −9.770 | 51.742 | 1.00 | 68.18 | A |
| ATOM | 1020 | C | GLN | A | 157 | −34.549 | −6.748 | 51.915 | 1.00 | 55.57 | A |
| ATOM | 1021 | O | GLN | A | 157 | −35.592 | −6.500 | 52.523 | 1.00 | 58.25 | A |
| ATOM | 1022 | N | ARG | A | 158 | −33.348 | −6.454 | 52.405 | 1.00 | 56.95 | A |
| ATOM | 1023 | CA | ARG | A | 158 | −33.219 | −5.878 | 53.740 | 1.00 | 58.18 | A |
| ATOM | 1024 | CB | ARG | A | 158 | −31.746 | −5.670 | 54.120 | 1.00 | 60.47 | A |
| ATOM | 1025 | CG | ARG | A | 158 | −30.974 | −4.795 | 53.183 | 1.00 | 63.47 | A |
| ATOM | 1026 | CD | ARG | A | 158 | −29.637 | −4.363 | 53.767 | .00 | 64.68 | A |
| ATOM | 1027 | NE | ARG | A | 158 | −28.781 | −5.485 | 54.146 | .00 | 66.13 | A |
| ATOM | 1028 | CZ | ARG | A | 158 | −28.460 | −6.495 | 53.343 | .00 | 66.77 | A |
| ATOM | 1029 | NH1 | ARG | A | 158 | −28.929 | −6.540 | 52.104 | .00 | 67.16 | A |
| ATOM | 1030 | NH2 | ARG | A | 158 | −27.658 | −7.457 | 53.777 | .00 | 67.18 | A |
| ATOM | 1031 | C | ARG | A | 158 | −33.981 | −4.583 | 53.955 | 1.00 | 56.33 | A |
| ATOM | 1032 | O | ARG | A | 158 | −34.381 | −4.283 | 55.074 | 1.00 | 57.22 | A |
| ATOM | 1033 | N | HIS | A | 159 | −34.200 | −3.824 | 52.894 | 1.00 | 53.92 | A |
| ATOM | 1034 | CA | HIS | A | 159 | −34.921 | −2.570 | 53.035 | 1.00 | 54.04 | A |
| ATOM | 1035 | CB | HIS | A | 159 | −34.484 | −1.576 | 51.960 | 1.00 | 54.66 | A |
| ATOM | 1036 | CG | HIS | A | 159 | −33.345 | −.697 | 52.370 | 1.00 | 57.91 | A |
| ATOM | 1037 | CD2 | HIS | A | 159 | −32.026 | −.962 | 52.533 | 1.00 | 58.35 | A |
| ATOM | 1038 | ND1 | HIS | A | 159 | −33.513 | .631 | 52.704 | 1.00 | 57.72 | A |
| ATOM | 1039 | CE1 | HIS | A | 159 | −32.348 | 1.142 | 53.059 | 1.00 | 58.36 | A |
| ATOM | 1040 | NE2 | HIS | A | 159 | −31.429 | .196 | 52.964 | 1.00 | 58.52 | A |
| ATOM | 1041 | C | HIS | A | 159 | −36.442 | −2.670 | 53.007 | 1.00 | 53.30 | A |
| ATOM | 1042 | O | HIS | A | 159 | −37.115 | −2.137 | 53.886 | 1.00 | 53.71 | A |
| ATOM | 1043 | N | PHE | A | 160 | −36.994 | −3.351 | 52.007 | 1.00 | 52.82 | A |
| ATOM | 1044 | CA | PHE | A | 160 | −38.441 | −3.421 | 51.898 | 1.00 | 49.84 | A |
| ATOM | 1045 | CB | PHE | A | 160 | −38.889 | −2.764 | 50.600 | 1.00 | 46.06 | A |
| ATOM | 1046 | CG | PHE | A | 160 | −38.498 | −1.320 | 50.495 | 1.00 | 42.91 | A |
| ATOM | 1047 | CD1 | PHE | A | 160 | −37.263 | −.959 | 50.009 | 1.00 | 40.66 | A |
| ATOM | 1048 | CD2 | PHE | A | 160 | −39.367 | −.318 | 50.915 | 1.00 | 44.06 | A |
| ATOM | 1049 | CE1 | PHE | A | 160 | −36.891 | .378 | 49.939 | 1.00 | 40.94 | A |
| ATOM | 1050 | CE2 | PHE | A | 160 | −39.003 | 1.016 | 50.848 | 1.00 | 41.70 | A |
| ATOM | 1051 | CZ | PHE | A | 160 | −37.757 | 1.363 | 50.357 | 1.00 | 40.48 | A |
| ATOM | 1052 | C | PHE | A | 160 | −39.079 | −4.781 | 52.030 | 1.00 | 49.99 | A |
| ATOM | 1053 | O | PHE | A | 160 | −40.307 | −4.884 | 52.043 | 1.00 | 51.75 | A |
| ATOM | 1054 | N | GLY | A | 161 | −38.264 | −5.821 | 52.138 | 1.00 | 48.57 | A |
| ATOM | 1055 | CA | GLY | A | 161 | −38.821 | −7.148 | 52.296 | 1.00 | 49.19 | A |
| ATOM | 1056 | C | GLY | A | 161 | −38.813 | −8.007 | 51.055 | 1.00 | 49.18 | A |
| ATOM | 1057 | O | GLY | A | 161 | −38.579 | −7.519 | 49.963 | 1.00 | 51.43 | A |
| ATOM | 1058 | N | LYS | A | 162 | −39.100 | −9.291 | 51.229 | 1.00 | 48.94 | A |
| ATOM | 1059 | CA | LYS | A | 162 | −39.099 | −10.241 | 50.132 | 1.00 | 49.10 | A |
| ATOM | 1060 | CB | LYS | A | 162 | −39.097 | −11.653 | 50.696 | 1.00 | 51.03 | A |
| ATOM | 1061 | CG | LYS | A | 162 | −38.161 | −12.598 | 49.972 | 1.00 | 53.23 | A |
| ATOM | 1062 | CD | LYS | A | 162 | −38.233 | −14.010 | 50.553 | 1.00 | 57.31 | A |
| ATOM | 1063 | CE | LYS | A | 162 | −39.548 | −14.709 | 50.214 | 1.00 | 58.76 | A |
| ATOM | 1064 | NZ | LYS | A | 162 | −39.684 | −14.932 | 48.750 | 1.00 | 59.79 | A |
| ATOM | 1065 | C | LYS | A | 162 | −40.225 | −10.106 | 49.097 | 1.00 | 48.64 | A |
| ATOM | 1066 | O | LYS | A | 162 | −39.981 | −10.233 | 47.900 | 1.00 | 47.81 | A |
| ATOM | 1067 | N | GLU | A | 163 | −41.453 | −9.867 | 49.542 | 1.00 | 48.44 | A |
| ATOM | 1068 | CA | GLU | A | 163 | −42.561 | −9.736 | 48.603 | 1.00 | 46.94 | A |
| ATOM | 1069 | CB | GLU | A | 163 | −43.844 | −9.375 | 49.349 | 1.00 | 46.69 | A |
| ATOM | 1070 | CG | GLU | A | 163 | −45.077 | −9.331 | 48.487 | 1.00 | 51.84 | A |
| ATOM | 1071 | CD | GLU | A | 163 | −46.286 | −8.791 | 49.246 | 1.00 | 58.11 | A |
| ATOM | 1072 | OE1 | GLU | A | 163 | −47.423 | −8.875 | 48.716 | 1.00 | 60.86 | A |
| ATOM | 1073 | OE2 | GLU | A | 163 | −46.101 | −8.275 | 50.374 | 1.00 | 58.19 | A |
| ATOM | 1074 | C | GLU | A | 163 | −42.209 | −8.651 | 47.585 | 1.00 | 46.29 | A |
| ATOM | 1075 | O | GLU | A | 163 | −42.391 | −8.843 | 46.384 | 1.00 | 44.01 | A |
| ATOM | 1076 | N | VAL | A | 164 | −41.676 | −7.528 | 48.076 | 1.00 | 45.87 | A |
| ATOM | 1077 | CA | VAL | A | 164 | −41.298 | −6.403 | 47.229 | 1.00 | 43.89 | A |
| ATOM | 1078 | CB | VAL | A | 164 | −40.990 | −5.156 | 48.081 | 1.00 | 39.97 | A |
| ATOM | 1079 | CG1 | VAL | A | 164 | −40.234 | −4.122 | 47.267 | 1.00 | 38.68 | A |
| ATOM | 1080 | CG2 | VAL | A | 164 | −42.278 | −4.568 | 48.564 | 1.00 | 35.59 | A |
| ATOM | 1081 | C | VAL | A | 164 | −40.121 | −6.730 | 46.303 | 1.00 | 45.81 | A |
| ATOM | 1082 | O | VAL | A | 164 | −39.948 | −6.099 | 45.247 | 1.00 | 46.86 | A |
| ATOM | 1083 | N | VAL | A | 165 | −39.305 | −7.707 | 46.672 | 1.00 | 45.04 | A |
| ATOM | 1084 | CA | VAL | A | 165 | −38.217 | −8.058 | 45.776 | 1.00 | 46.37 | A |
| ATOM | 1085 | CB | VAL | A | 165 | −37.031 | −8.702 | 46.499 | 1.00 | 45.93 | A |
| ATOM | 1086 | CG1 | VAL | A | 165 | −36.072 | −9.314 | 45.492 | 1.00 | 40.65 | A |
| ATOM | 1087 | CG2 | VAL | A | 165 | −36.299 | −7.638 | 47.315 | 1.00 | 47.35 | A |
| ATOM | 1088 | C | VAL | A | 165 | −38.732 | −9.030 | 44.747 | 1.00 | 48.10 | A |
| ATOM | 1089 | O | VAL | A | 165 | −38.351 | −8.969 | 43.587 | 1.00 | 52.34 | A |
| ATOM | 1090 | N | ASP | A | 166 | −39.622 | −9.911 | 45.166 | 1.00 | 47.89 | A |
| ATOM | 1091 | CA | ASP | A | 166 | −40.167 | −10.902 | 44.267 | 1.00 | 48.14 | A |
| ATOM | 1092 | CB | ASP | A | 166 | −40.816 | −12.031 | 45.067 | 1.00 | 49.59 | A |
| ATOM | 1093 | CG | ASP | A | 166 | −39.797 | −12.862 | 45.836 | 1.00 | 50.22 | A |
| ATOM | 1094 | OD1 | ASP | A | 166 | −40.135 | −13.369 | 46.934 | 1.00 | 50.92 | A |
| ATOM | 1095 | OD2 | ASP | A | 166 | −38.664 | −13.011 | 45.332 | 1.00 | 47.32 | A |
| ATOM | 1096 | C | ASP | A | 166 | −41.160 | −10.380 | 43.250 | 1.00 | 49.17 | A |
| ATOM | 1097 | O | ASP | A | 166 | −41.076 | −10.734 | 42.081 | 1.00 | 51.58 | A |
| ATOM | 1098 | N | TYR | A | 167 | −42.101 | −9.537 | 43.659 | 1.00 | 49.45 | A |
| ATOM | 1099 | CA | TYR | A | 167 | −43.101 | −9.078 | 42.698 | 1.00 | 47.07 | A |
| ATOM | 1100 | CB | TYR | A | 167 | −44.506 | −9.415 | 43.199 | 1.00 | 47.73 | A |
| ATOM | 1101 | CG | TYR | A | 167 | −44.669 | −10.900 | 43.418 | 1.00 | 50.80 | A |
| ATOM | 1102 | CD1 | TYR | A | 167 | −44.474 | −11.466 | 44.676 | 1.00 | 52.84 | A |
| ATOM | 1103 | CE1 | TYR | A | 167 | −44.528 | −12.842 | 44.869 | 1.00 | 53.24 | A |
| ATOM | 1104 | CD2 | TYR | A | 167 | −44.930 | −11.755 | 42.353 | 1.00 | 52.75 | A |
| ATOM | 1105 | CE2 | TYR | A | 167 | −44.990 | −13.138 | 42.533 | 1.00 | 53.29 | A |
| ATOM | 1106 | CZ | TYR | A | 167 | −44.786 | −13.676 | 43.797 | 1.00 | 54.66 | A |
| ATOM | 1107 | OH | TYR | A | 167 | −44.840 | −15.047 | 43.992 | 1.00 | 55.97 | A |
| ATOM | 1108 | C | TYR | A | 167 | −43.019 | −7.641 | 42.273 | 1.00 | 45.33 | A |
| ATOM | 1109 | O | TYR | A | 167 | −43.904 | −7.145 | 41.610 | 1.00 | 44.86 | A |
| ATOM | 1110 | N | LEU | A | 168 | −41.940 | −6.972 | 42.635 | 1.00 | 46.00 | A |
| ATOM | 1111 | CA | LEU | A | 168 | −41.757 | −5.593 | 42.215 | 1.00 | 45.87 | A |
| ATOM | 1112 | CB | LEU | A | 168 | −41.949 | −4.630 | 43.378 | 1.00 | 44.01 | A |
| ATOM | 1113 | CG | LEU | A | 168 | −43.409 | −4.339 | 43.690 | 1.00 | 41.85 | A |
| ATOM | 1114 | CD1 | LEU | A | 168 | −43.486 | −3.364 | 44.836 | 1.00 | 42.73 | A |
| ATOM | 1115 | CD2 | LEU | A | 168 | −44.081 | −3.765 | 42.467 | 1.00 | 41.26 | A |
| ATOM | 1116 | C | LEU | A | 168 | −40.396 | −5.376 | 41.579 | 1.00 | 46.72 | A |
| ATOM | 1117 | O | LEU | A | 168 | −40.298 | −5.253 | 40.360 | 1.00 | 47.27 | A |
| ATOM | 1118 | N | ILE | A | 169 | −39.332 | −5.364 | 42.366 | 1.00 | 47.35 | A |
| ATOM | 1119 | CA | ILE | A | 169 | −38.057 | −5.114 | 41.727 | 1.00 | 48.57 | A |
| ATOM | 1120 | CB | ILE | A | 169 | −36.932 | −4.762 | 42.755 | 1.00 | 47.43 | A |
| ATOM | 1121 | CG2 | ILE | A | 169 | −37.440 | −3.792 | 43.785 | 1.00 | 46.94 | A |
| ATOM | 1122 | CG1 | ILE | A | 169 | −36.378 | −6.018 | 43.376 | 1.00 | 47.27 | A |
| ATOM | 1123 | CD1 | ILE | A | 169 | −35.239 | −6.546 | 42.573 | 1.00 | 50.46 | A |
| ATOM | 1124 | C | ILE | A | 169 | −37.625 | −6.248 | 40.793 | 1.00 | 48.40 | A |
| ATOM | 1125 | O | ILE | A | 169 | −36.928 | −6.000 | 39.816 | 1.00 | 48.80 | A |
| ATOM | 1126 | N | ASP | A | 170 | −38.039 | −7.482 | 41.058 | 1.00 | 49.30 | A |
| ATOM | 1127 | CA | ASP | A | 170 | −37.653 | −8.583 | 40.164 | 1.00 | 50.50 | A |
| ATOM | 1128 | CB | ASP | A | 170 | −38.051 | −9.936 | 40.760 | 1.00 | 51.71 | A |
| ATOM | 1129 | CG | ASP | A | 170 | −37.794 | −11.095 | 39.806 | 1.00 | 54.14 | A |
| ATOM | 1130 | OD1 | ASP | A | 170 | −38.476 | −11.184 | 38.765 | 1.00 | 56.54 | A |
| ATOM | 1131 | OD2 | ASP | A | 170 | −36.905 | −11.920 | 40.087 | 1.00 | 54.53 | A |
| ATOM | 1132 | C | ASP | A | 170 | −38.275 | −8.419 | 38.765 | 1.00 | 50.76 | A |
| ATOM | 1133 | O | ASP | A | 170 | −37.583 | −8.516 | 37.755 | 1.00 | 49.76 | A |
| ATOM | 1134 | N | PRO | A | 171 | −39.595 | −8.193 | 38.688 | 1.00 | 50.62 | A |
| ATOM | 1135 | CD | PRO | A | 171 | −40.593 | −8.166 | 39.770 | 1.00 | 50.59 | A |
| ATOM | 1136 | CA | PRO | A | 171 | −40.235 | −8.015 | 37.382 | 1.00 | 49.28 | A |
| ATOM | 1137 | CB | PRO | A | 171 | −41.685 | −7.708 | 37.740 | 1.00 | 49.76 | A |
| ATOM | 1138 | CG | PRO | A | 171 | −41.877 | −8.393 | 39.024 | 1.00 | 51.07 | A |
| ATOM | 1139 | C | PRO | A | 171 | −39.596 | −6.830 | 36.659 | 1.00 | 49.16 | A |
| ATOM | 1140 | O | PRO | A | 171 | −39.510 | −6.815 | 35.436 | 1.00 | 50.31 | A |
| ATOM | 1141 | N | PHE | A | 172 | −39.173 | −5.825 | 37.422 | 1.00 | 47.69 | A |
| ATOM | 1142 | CA | PHE | A | 172 | −38.555 | −4.641 | 36.840 | 1.00 | 47.09 | A |
| ATOM | 1143 | CB | PHE | A | 172 | −38.392 | −3.550 | 37.897 | 1.00 | 47.14 | A |
| ATOM | 1144 | CG | PHE | A | 172 | −37.580 | −2.364 | 37.441 | 1.00 | 47.32 | A |
| ATOM | 1145 | CD1 | PHE | A | 172 | −36.185 | −2.388 | 37.485 | 1.00 | 47.80 | A |
| ATOM | 1146 | CD2 | PHE | A | 172 | −38.205 | −1.222 | 36.959 | 1.00 | 47.92 | A |
| ATOM | 1147 | CE1 | PHE | A | 172 | −35.435 | −1.296 | 37.056 | 1.00 | 46.09 | A |
| ATOM | 1148 | CE2 | PHE | A | 172 | −37.452 | −.121 | 36.527 | 1.00 | 46.51 | A |
| ATOM | 1149 | CZ | PHE | A | 172 | −36.072 | −.164 | 36.578 | 1.00 | 44.34 | A |
| ATOM | 1150 | C | PHE | A | 172 | −37.212 | −4.971 | 36.211 | 1.00 | 46.11 | A |
| ATOM | 1151 | O | PHE | A | 172 | −36.896 | −4.496 | 35.129 | 1.00 | 47.65 | A |
| ATOM | 1152 | N | VAL | A | 173 | −36.412 | −5.771 | 36.892 | 1.00 | 44.55 | A |
| ATOM | 1153 | CA | VAL | A | 173 | −35.128 | −6.156 | 36.348 | 1.00 | 43.83 | A |
| ATOM | 1154 | CB | VAL | A | 173 | −34.281 | −6.814 | 37.430 | 1.00 | 41.91 | A |
| ATOM | 1155 | CG1 | VAL | A | 173 | −33.112 | −7.531 | 36.826 | 1.00 | 41.98 | A |
| ATOM | 1156 | CG2 | VAL | A | 173 | −33.810 | −5.741 | 38.393 | 1.00 | 40.87 | A |
| ATOM | 1157 | C | VAL | A | 173 | −35.348 | −7.095 | 35.155 | 1.00 | 44.84 | A |
| ATOM | 1158 | O | VAL | A | 173 | −34.669 | −6.987 | 34.131 | 1.00 | 45.85 | A |
| ATOM | 1159 | N | ALA | A | 174 | −36.316 | −7.995 | 35.266 | 1.00 | 44.85 | A |
| ATOM | 1160 | CA | ALA | A | 174 | −36.600 | −8.906 | 34.166 | 1.00 | 46.65 | A |
| ATOM | 1161 | CB | ALA | A | 174 | −37.718 | −9.873 | 34.540 | 1.00 | 47.85 | A |
| ATOM | 1162 | C | ALA | A | 174 | −37.002 | −8.111 | 32.933 | 1.00 | 46.74 | A |
| ATOM | 1163 | O | ALA | A | 174 | −36.649 | −8.457 | 31.813 | 1.00 | 46.60 | A |
| ATOM | 1164 | N | GLY | A | 175 | −37.741 | −7.037 | 33.145 | 1.00 | 47.41 | A |
| ATOM | 1165 | CA | GLY | A | 175 | −38.178 | −6.232 | 32.024 | 1.00 | 49.86 | A |
| ATOM | 1166 | C | GLY | A | 175 | −37.130 | −5.280 | 31.485 | 1.00 | 51.07 | A |
| ATOM | 1167 | O | GLY | A | 175 | −37.352 | −4.621 | 30.468 | 1.00 | 50.75 | A |
| ATOM | 1168 | N | THR | A | 176 | −35.986 | −5.202 | 32.153 | 1.00 | 52.03 | A |
| ATOM | 1169 | CA | THR | A | 176 | −34.937 | −4.301 | 31.701 | 1.00 | 54.12 | A |
| ATOM | 1170 | CB | THR | A | 176 | −34.386 | −3.459 | 32.866 | 1.00 | 55.03 | A |
| ATOM | 1171 | OG1 | THR | A | 176 | −35.458 | −2.729 | 33.474 | 1.00 | 57.05 | A |
| ATOM | 1172 | CG2 | THR | A | 176 | −33.343 | −2.476 | 32.362 | 1.00 | 55.43 | A |
| ATOM | 1173 | C | THR | A | 176 | −33.765 | −5.006 | 31.014 | 1.00 | 54.51 | A |
| ATOM | 1174 | O | THR | A | 176 | −33.183 | −4.456 | 30.082 | 1.00 | 54.42 | A |
| ATOM | 1175 | N | CYS | A | 177 | −33.427 | −6.213 | 31.473 | 1.00 | 53.39 | A |
| ATOM | 1176 | CA | CYS | A | 177 | −32.320 | −6.967 | 30.901 | 1.00 | 51.24 | A |
| ATOM | 1177 | CB | CYS | A | 177 | −31.045 | −6.655 | 31.669 | 1.00 | 51.22 | A |
| ATOM | 1178 | SG | CYS | A | 177 | −31.162 | −7.023 | 33.435 | 1.00 | 50.94 | A |
| ATOM | 1179 | C | CYS | A | 177 | −32.561 | −8.470 | 30.926 | 1.00 | 51.53 | A |
| ATOM | 1180 | O | CYS | A | 177 | −31.727 | −9.236 | 30.450 | 1.00 | 52.90 | A |
| ATOM | 1181 | N | GLY | A | 178 | −33.694 | −8.887 | 31.487 | 1.00 | 50.77 | A |
| ATOM | 1182 | CA | GLY | A | 178 | −34.022 | −10.300 | 31.571 | 1.00 | 49.44 | A |
| ATOM | 1183 | C | GLY | A | 178 | −33.184 | −11.002 | 32.617 | 1.00 | 50.01 | A |
| ATOM | 1184 | O | GLY | A | 178 | −33.178 | −12.226 | 32.709 | 1.00 | 49.95 | A |
| ATOM | 1185 | N | GLY | A | 179 | −32.486 | −10.212 | 33.427 | 1.00 | 51.17 | A |
| ATOM | 1186 | CA | GLY | A | 179 | −31.611 | −10.762 | 34.450 | 1.00 | 52.67 | A |
| ATOM | 1187 | C | GLY | A | 179 | −32.253 | −11.138 | 35.767 | 1.00 | 53.42 | A |
| ATOM | 1188 | O | GLY | A | 179 | −33.477 | −11.092 | 35.919 | 1.00 | 53.10 | A |
| ATOM | 1189 | N | ASP | A | 180 | −31.413 | −11.510 | 36.728 | 1.00 | 53.28 | A |
| ATOM | 1190 | CA | ASP | A | 180 | −31.890 | −11.914 | 38.036 | 1.00 | 54.48 | A |
| ATOM | 1191 | CB | ASP | A | 180 | −31.541 | −13.396 | 38.258 | 1.00 | 56.97 | A |
| ATOM | 1192 | CG | ASP | A | 180 | −31.400 | −13.767 | 39.731 | 1.00 | 60.15 | A |
| ATOM | 1193 | OD1 | ASP | A | 180 | −30.278 | −13.632 | 40.258 | 1.00 | 61.14 | A |
| ATOM | 1194 | OD2 | ASP | A | 180 | −32.399 | −14.191 | 40.364 | 1.00 | 63.18 | A |
| ATOM | 1195 | C | ASP | A | 180 | −31.321 | −11.018 | 39.137 | 1.00 | 54.23 | A |
| ATOM | 1196 | O | ASP | A | 180 | −30.108 | −10.851 | 39.253 | 1.00 | 55.56 | A |
| ATOM | 1197 | N | PRO | A | 181 | −32.200 | −10.438 | 39.970 | 1.00 | 53.03 | A |
| ATOM | 1198 | CD | PRO | A | 181 | −33.625 | −10.787 | 40.058 | 1.00 | 51.78 | A |
| ATOM | 1199 | CA | PRO | A | 181 | −32.836 | −9.545 | 41.072 | 1.00 | 52.78 | A |
| ATOM | 1200 | CB | PRO | A | 181 | −33.120 | −9.466 | 41.884 | 1.00 | 50.50 | A |
| ATOM | 1201 | CG | PRO | A | 181 | −34.162 | −9.663 | 40.880 | 1.00 | 51.51 | A |
| ATOM | 1202 | C | PRO | A | 181 | −30.669 | −10.032 | 41.918 | 1.00 | 53.52 | A |
| ATOM | 1203 | O | PRO | A | 181 | −29.830 | −9.242 | 42.340 | 1.00 | 53.28 | A |
| ATOM | 1204 | N | ASP | A | 182 | −30.627 | −11.338 | 42.159 | 1.00 | 54.86 | A |
| ATOM | 1205 | CA | ASP | A | 182 | −29.592 | −11.943 | 42.986 | 1.00 | 56.46 | A |
| ATOM | 1206 | CB | ASP | A | 182 | −29.925 | −13.420 | 43.274 | 1.00 | 55.42 | A |
| ATOM | 1207 | CG | ASP | A | 182 | −31.282 | −13.603 | 43.933 | .00 | 56.51 | A |
| ATOM | 1208 | OD1 | ASP | A | 182 | −31.475 | −14.632 | 44.615 | .00 | 56.59 | A |
| ATOM | 1209 | OD2 | ASP | A | 182 | −32.159 | −12.731 | 43.761 | .00 | 56.68 | A |
| ATOM | 1210 | C | ASP | A | 182 | −28.193 | −11.849 | 42.392 | 1.00 | 57.46 | A |
| ATOM | 1211 | O | ASP | A | 182 | −27.199 | −11.997 | 43.103 | 1.00 | 59.25 | A |
| ATOM | 1212 | N | SER | A | 183 | −28.097 | −11.584 | 41.100 | 1.00 | 57.95 | A |
| ATOM | 1213 | CA | SER | A | 183 | −26.785 | −11.520 | 40.498 | 1.00 | 59.59 | A |
| ATOM | 1214 | CB | SER | A | 183 | −26.620 | −12.670 | 39.516 | 1.00 | 61.09 | A |
| ATOM | 1215 | OG | SER | A | 183 | −27.688 | −12.680 | 38.584 | 1.00 | 63.75 | A |
| ATOM | 1216 | C | SER | A | 183 | −26.475 | −10.210 | 39.816 | 1.00 | 59.60 | A |
| ATOM | 1217 | O | SER | A | 183 | −25.833 | −10.184 | 38.774 | 1.00 | 60.76 | A |
| ATOM | 1218 | N | LEU | A | 184 | −26.924 | −9.118 | 40.413 | 1.00 | 60.67 | A |
| ATOM | 1219 | CA | LEU | A | 184 | −26.673 | −7.785 | 39.875 | 1.00 | 60.91 | A |
| ATOM | 1220 | CB | LEU | A | 184 | −27.957 | −7.171 | 39.325 | 1.00 | 58.98 | A |
| ATOM | 1221 | CG | LEU | A | 184 | −28.253 | −7.396 | 37.851 | 1.00 | 57.86 | A |
| ATOM | 1222 | CD1 | LEU | A | 184 | −28.210 | −8.861 | 37.536 | 1.00 | 60.18 | A |
| ATOM | 1223 | CD2 | LEU | A | 184 | −29.609 | −6.832 | 37.527 | 1.00 | 58.56 | A |
| ATOM | 1224 | C | LEU | A | 184 | −26.113 | −6.866 | 40.945 | 1.00 | 61.74 | A |
| ATOM | 1225 | O | LEU | A | 184 | −26.614 | −6.829 | 42.063 | 1.00 | 61.26 | A |
| ATOM | 1226 | N | SER | A | 185 | −25.066 | −6.130 | 40.605 | 1.00 | 63.92 | A |
| ATOM | 1227 | CA | SER | A | 185 | −24.487 | −5.206 | 41.559 | 1.00 | 67.00 | A |
| ATOM | 1228 | CB | SER | A | 185 | −23.236 | −4.537 | 40.980 | 1.00 | 68.14 | A |
| ATOM | 1229 | OG | SER | A | 185 | −22.760 | −3.499 | 41.828 | 1.00 | 70.03 | A |
| ATOM | 1230 | C | SER | A | 185 | −25.551 | −4.159 | 41.826 | 1.00 | 67.96 | A |
| ATOM | 1231 | O | SER | A | 185 | −26.096 | −3.573 | 40.902 | 1.00 | 67.31 | A |
| ATOM | 1232 | N | MSE | A | 186 | −25.868 | −3.945 | 43.092 | 1.00 | 70.19 | A |
| ATOM | 1233 | CA | MSE | A | 186 | −26.861 | −2.946 | 43.438 | 1.00 | 71.88 | A |
| ATOM | 1234 | CB | MSE | A | 186 | −27.134 | −2.991 | 44.938 | 1.00 | 74.08 | A |
| ATOM | 1235 | CG | MSE | A | 186 | −28.004 | −1.873 | 45.442 | 1.00 | 76.57 | A |
| ATOM | 1236 | SE | MSE | A | 186 | −29.709 | −1.793 | 44.546 | 1.00 | 81.72 | A |
| ATOM | 1237 | CE | MSE | A | 186 | −30.106 | .058 | 44.942 | 1.00 | 78.12 | A |
| ATOM | 1238 | C | MSE | A | 186 | −26.266 | −1.602 | 43.043 | 1.00 | 71.81 | A |
| ATOM | 1239 | O | MSE | A | 186 | −26.915 | −.772 | 42.403 | 1.00 | 69.67 | A |
| ATOM | 1240 | N | HIS | A | 187 | −25.004 | −1.424 | 43.416 | 1.00 | 72.90 | A |
| ATOM | 1241 | CA | HIS | A | 187 | −24.267 | −.205 | 43.131 | 1.00 | 73.98 | A |
| ATOM | 1242 | CB | HIS | A | 187 | −22.814 | −.374 | 43.591 | 1.00 | 76.04 | A |
| ATOM | 1243 | CG | HIS | A | 187 | −21.960 | .833 | 43.355 | 1.00 | 78.10 | A |
| ATOM | 1244 | CD2 | HIS | A | 187 | −20.695 | .951 | 42.888 | 1.00 | 79.43 | A |
| ATOM | 1245 | ND1 | HIS | A | 187 | −22.387 | 2.116 | 43.630 | 1.00 | 78.63 | A |
| ATOM | 1246 | CE1 | HIS | A | 187 | −21.424 | 2.971 | 43.341 | 1.00 | 78.97 | A |
| ATOM | 1247 | NE2 | HIS | A | 187 | −20.386 | 2.290 | 42.889 | 1.00 | 80.63 | A |
| ATOM | 1248 | C | HIS | A | 187 | −24.306 | .158 | 41.654 | 1.00 | 73.21 | A |
| ATOM | 1249 | O | HIS | A | 187 | −24.706 | 1.261 | 41.278 | 1.00 | 71.85 | A |
| ATOM | 1250 | N | ALA | A | 188 | −23.903 | −.784 | 40.817 | 1.00 | 73.52 | A |
| ATOM | 1251 | CA | ALA | A | 188 | −23.860 | −.544 | 39.386 | 1.00 | 74.36 | A |
| ATOM | 1252 | CB | ALA | A | 188 | −22.899 | −1.534 | 38.740 | 1.00 | 75.17 | A |
| ATOM | 1253 | C | ALA | A | 188 | −25.211 | −.552 | 38.661 | 1.00 | 74.03 | A |
| ATOM | 1254 | O | ALA | A | 188 | −25.356 | .083 | 37.628 | 1.00 | 74.24 | A |
| ATOM | 1255 | N | SER | A | 189 | −26.202 | −1.260 | 39.185 | 1.00 | 74.79 | A |
| ATOM | 1256 | CA | SER | A | 189 | −27.507 | −1.300 | 38.531 | 1.00 | 74.29 | A |
| ATOM | 1257 | CB | SER | A | 189 | −28.270 | −2.562 | 38.924 | 1.00 | 74.12 | A |
| ATOM | 1258 | OG | SER | A | 189 | −27.649 | −3.712 | 38.388 | 1.00 | 74.41 | A |
| ATOM | 1259 | C | SER | A | 189 | −28.356 | −.080 | 38.852 | 1.00 | 75.16 | A |
| ATOM | 1260 | O | SER | A | 189 | −28.939 | .511 | 37.952 | 1.00 | 75.51 | A |
| ATOM | 1261 | N | PHE | A | 190 | −28.438 | .292 | 40.129 | 1.00 | 75.93 | A |
| ATOM | 1262 | CA | PHE | A | 190 | −29.219 | 1.466 | 40.537 | 1.00 | 76.39 | A |
| ATOM | 1263 | CB | PHE | A | 190 | −30.323 | 1.087 | 41.519 | 1.00 | 75.66 | A |
| ATOM | 1264 | CG | PHE | A | 190 | −31.293 | .089 | 40.990 | 1.00 | 74.81 | A |
| ATOM | 1265 | CD1 | PHE | A | 190 | −30.936 | −1.241 | 40.857 | 1.00 | 74.46 | A |
| ATOM | 1266 | CD2 | PHE | A | 190 | −32.579 | .475 | 40.646 | 1.00 | 75.18 | A |
| ATOM | 1267 | CE1 | PHE | A | 190 | −31.848 | −2.177 | 40.390 | 1.00 | 74.95 | A |
| ATOM | 1268 | CE2 | PHE | A | 190 | −33.502 | −.453 | 40.178 | 1.00 | 75.35 | A |
| ATOM | 1269 | CZ | PHE | A | 190 | −33.137 | −1.781 | 40.049 | 1.00 | 75.06 | A |
| ATOM | 1270 | C | PHE | A | 190 | −28.324 | 2.498 | 41.215 | 1.00 | 77.10 | A |
| ATOM | 1271 | O | PHE | A | 190 | −28.320 | 2.616 | 42.443 | 1.00 | 77.15 | A |
| ATOM | 1272 | N | PRO | A | 191 | −27.568 | 3.272 | 40.426 | 1.00 | 77.44 | A |
| ATOM | 1273 | CD | PRO | A | 191 | −27.469 | 3.240 | 38.961 | 1.00 | 77.02 | A |
| ATOM | 1274 | CA | PRO | A | 191 | −26.670 | 4.287 | 40.982 | 1.00 | 78.29 | A |
| ATOM | 1275 | CB | PRO | A | 191 | −25.967 | 4.836 | 39.747 | 1.00 | 78.28 | A |
| ATOM | 1276 | CG | PRO | A | 191 | −26.065 | 3.705 | 38.758 | 1.00 | 78.46 | A |
| ATOM | 1277 | C | PRO | A | 191 | −27.435 | 5.368 | 41.726 | 1.00 | 79.04 | A |
| ATOM | 1278 | O | PRO | A | 191 | −27.065 | 5.764 | 42.837 | 1.00 | 78.95 | A |
| ATOM | 1279 | N | ALA | A | 192 | −28.512 | 5.827 | 41.097 | 1.00 | 80.06 | A |
| ATOM | 1280 | CA | ALA | A | 192 | −29.363 | 6.873 | 41.650 | 1.00 | 80.72 | A |
| ATOM | 1281 | CB | ALA | A | 192 | −30.510 | 7.165 | 40.694 | 1.00 | 81.59 | A |
| ATOM | 1282 | C | ALA | A | 192 | −29.914 | 6.510 | 43.020 | 1.00 | 81.35 | A |
| ATOM | 1283 | O | ALA | A | 192 | −30.500 | 7.352 | 43.697 | 1.00 | 81.16 | A |
| ATOM | 1284 | N | LEU | A | 193 | −29.742 | 5.258 | 43.430 | 1.00 | 81.60 | A |
| ATOM | 1285 | CA | LEU | A | 193 | −30.230 | 4.840 | 44.733 | 1.00 | 80.12 | A |
| ATOM | 1286 | CB | LEU | A | 193 | −31.223 | 3.686 | 44.594 | 1.00 | 79.05 | A |
| ATOM | 1287 | CG | LEU | A | 193 | −32.565 | 3.973 | 43.930 | 1.00 | 76.87 | A |
| ATOM | 1288 | CD1 | LEU | A | 193 | −33.444 | 2.754 | 44.032 | 1.00 | 77.33 | A |
| ATOM | 1289 | CD2 | LEU | A | 193 | −33.231 | 5.128 | 44.615 | 1.00 | 76.65 | A |
| ATOM | 1290 | C | LEU | A | 193 | −29.100 | 4.436 | 45.660 | 1.00 | 80.25 | A |
| ATOM | 1291 | O | LEU | A | 193 | −29.207 | 4.604 | 46.872 | 1.00 | 79.30 | A |
| ATOM | 1292 | N | TRP | A | 194 | −28.018 | 3.901 | 45.102 | 1.00 | 81.49 | A |
| ATOM | 1293 | CA | TRP | A | 194 | −26.902 | 3.493 | 45.945 | 1.00 | 84.42 | A |
| ATOM | 1294 | CB | TRP | A | 194 | −25.789 | 2.841 | 45.104 | 1.00 | 84.86 | A |
| ATOM | 1295 | CG | TRP | A | 194 | −24.499 | 2.589 | 45.861 | 1.00 | 86.81 | A |
| ATOM | 1296 | CD2 | TRP | A | 194 | −24.170 | 1.445 | 46.664 | 1.00 | 87.14 | A |
| ATOM | 1297 | CE2 | TRP | A | 194 | −22.866 | 1.656 | 47.179 | 1.00 | 88.10 | A |
| ATOM | 1298 | CE3 | TRP | A | 194 | −24.845 | .263 | 47.000 | 1.00 | 88.25 | A |
| ATOM | 1299 | CD1 | TRP | A | 194 | −23.411 | 3.423 | 45.927 | 1.00 | 88.00 | A |
| ATOM | 1300 | NE1 | TRP | A | 194 | −22.430 | 2.869 | 46.712 | 1.00 | 87.45 | A |
| ATOM | 1301 | CZ2 | TRP | A | 194 | −22.222 | .725 | 48.013 | 1.00 | 88.51 | A |
| ATOM | 1302 | CZ3 | TRP | A | 194 | −24.205 | −.666 | 47.832 | 1.00 | 89.03 | A |
| ATOM | 1303 | CH2 | TRP | A | 194 | −22.904 | −.425 | 48.327 | 1.00 | 89.20 | A |
| ATOM | 1304 | C | TRP | A | 194 | −26.398 | 4.734 | 46.671 | 1.00 | 85.22 | A |
| ATOM | 1305 | O | TRP | A | 194 | −26.063 | 4.697 | 47.862 | 1.00 | 83.85 | A |
| ATOM | 1306 | N | ASN | A | 195 | −26.380 | 5.844 | 45.945 | 1.00 | 86.66 | A |
| ATOM | 1307 | CA | ASN | A | 195 | −25.934 | 7.105 | 46.510 | 1.00 | 87.77 | A |
| ATOM | 1308 | CB | ASN | A | 195 | −25.964 | 8.192 | 45.437 | 1.00 | 88.78 | A |
| ATOM | 1309 | CG | ASN | A | 195 | −24.871 | 8.002 | 44.392 | 1.00 | 89.91 | A |
| ATOM | 1310 | OD1 | ASN | A | 195 | −23.681 | 8.105 | 44.701 | 1.00 | 90.64 | A |
| ATOM | 1311 | ND2 | ASN | A | 195 | −25.268 | 7.716 | 43.155 | 1.00 | 89.71 | A |
| ATOM | 1312 | C | ASN | A | 195 | −26.799 | 7.498 | 47.699 | 1.00 | 87.71 | A |
| ATOM | 1313 | O | ASN | A | 195 | −26.315 | 7.534 | 48.828 | 1.00 | 88.14 | A |
| ATOM | 1314 | N | LEU | A | 196 | −28.076 | 7.774 | 47.448 | 1.00 | 87.37 | A |
| ATOM | 1315 | CA | LEU | A | 196 | −28.996 | 8.158 | 48.514 | 1.00 | 87.82 | A |
| ATOM | 1316 | CB | LEU | A | 196 | −30.438 | 8.047 | 48.034 | 1.00 | 86.75 | A |
| ATOM | 1317 | CG | LEU | A | 196 | −31.002 | 9.334 | 47.444 | 1.00 | 87.31 | A |
| ATOM | 1318 | CD1 | LEU | A | 196 | −30.055 | 9.860 | 46.392 | 1.00 | 88.69 | A |
| ATOM | 1319 | CD2 | LEU | A | 196 | −32.375 | 9.076 | 46.859 | 1.00 | 87.30 | A |
| ATOM | 1320 | C | LEU | A | 196 | −28.835 | 7.369 | 49.812 | 1.00 | 88.94 | A |
| ATOM | 1321 | O | LEU | A | 196 | −28.942 | 7.939 | 50.901 | 1.00 | 88.97 | A |
| ATOM | 1322 | N | GLU | A | 197 | −28.586 | 6.067 | 49.718 | 1.00 | 89.25 | A |
| ATOM | 1323 | CA | GLU | A | 197 | −28.419 | 5.299 | 50.938 | 1.00 | 89.71 | A |
| ATOM | 1324 | CB | GLU | A | 197 | −28.326 | 3.800 | 50.661 | 1.00 | 89.32 | A |
| ATOM | 1325 | CG | GLU | A | 197 | −28.143 | 2.987 | 51.942 | 1.00 | 90.40 | A |
| ATOM | 1326 | CD | GLU | A | 197 | −27.637 | 1.576 | 51.705 | 1.00 | 91.69 | A |
| ATOM | 1327 | OE1 | GLU | A | 197 | −26.556 | 1.423 | 51.089 | 1.00 | 92.77 | A |
| ATOM | 1328 | OE2 | GLU | A | 197 | −28.314 | .617 | 52.142 | 1.00 | 91.82 | A |
| ATOM | 1329 | C | GLU | A | 197 | −27.146 | 5.756 | 51.639 | 1.00 | 90.23 | A |
| ATOM | 1330 | O | GLU | A | 197 | −27.135 | 5.949 | 52.859 | 1.00 | 91.49 | A |
| ATOM | 1331 | N | LYS | A | 198 | −26.073 | 5.929 | 50.870 | 1.00 | 89.82 | A |
| ATOM | 1332 | CA | LYS | A | 198 | −24.797 | 6.357 | 51.437 | 1.00 | 88.99 | A |
| ATOM | 1333 | CB | LYS | A | 198 | −23.769 | 6.572 | 50.323 | 1.00 | 89.00 | A |
| ATOM | 1334 | CG | LYS | A | 198 | −22.409 | 7.081 | 50.799 | 1.00 | 90.44 | A |
| ATOM | 1335 | CD | LYS | A | 198 | −22.424 | 8.584 | 51.079 | 1.00 | 91.43 | A |
| ATOM | 1336 | CE | LYS | A | 198 | −21.090 | 9.078 | 51.633 | 1.00 | 91.23 | A |
| ATOM | 1337 | NZ | LYS | A | 198 | −21.112 | 10.539 | 51.939 | 1.00 | 90.97 | A |
| ATOM | 1338 | C | LYS | A | 198 | −24.971 | 7.639 | 52.242 | 1.00 | 88.61 | A |
| ATOM | 1339 | O | LYS | A | 198 | −24.460 | 7.754 | 53.359 | 1.00 | 88.93 | A |
| ATOM | 1340 | N | ALA | A | 199 | −25.704 | 8.590 | 51.666 | 1.00 | 87.68 | A |
| ATOM | 1341 | CA | ALA | A | 199 | −25.964 | 9.882 | 52.297 | 1.00 | 86.12 | A |
| ATOM | 1342 | CB | ALA | A | 199 | −26.250 | 10.932 | 51.222 | 1.00 | 85.55 | A |
| ATOM | 1343 | C | ALA | A | 199 | −27.132 | 9.818 | 53.285 | 1.00 | 85.09 | A |
| ATOM | 1344 | O | ALA | A | 199 | −26.935 | 9.695 | 54.501 | 1.00 | 85.17 | A |
| ATOM | 1345 | N | PHE | A | 200 | −28.346 | 9.894 | 52.747 | 1.00 | 83.00 | A |
| ATOM | 1346 | CA | PHE | A | 200 | −29.562 | 9.865 | 53.550 | 1.00 | 80.37 | A |
| ATOM | 1347 | CB | PHE | A | 200 | −30.760 | 10.122 | 52.656 | 1.00 | 79.86 | A |
| ATOM | 1348 | CG | PHE | A | 200 | −30.736 | 11.470 | 52.010 | 1.00 | 80.04 | A |
| ATOM | 1349 | CD1 | PHE | A | 200 | −30.892 | 12.618 | 52.772 | 1.00 | 79.87 | A |
| ATOM | 1350 | CD2 | PHE | A | 200 | −30.559 | 11.593 | 50.637 | 1.00 | 81.00 | A |
| ATOM | 1351 | CE1 | PHE | A | 200 | −30.875 | 13.873 | 52.178 | 1.00 | 80.68 | A |
| ATOM | 1352 | CE2 | PHE | A | 200 | −30.540 | 12.839 | 50.030 | 1.00 | 81.04 | A |
| ATOM | 1353 | CZ | PHE | A | 200 | −30.700 | 13.985 | 50.804 | 1.00 | 81.40 | A |
| ATOM | 1354 | C | PHE | A | 200 | −29.799 | 8.608 | 54.377 | 1.00 | 78.91 | A |
| ATOM | 1355 | O | PHE | A | 200 | −30.462 | 8.676 | 55.407 | 1.00 | 79.58 | A |
| ATOM | 1356 | N | GLY | A | 201 | −29.285 | 7.466 | 53.925 | 1.00 | 76.87 | A |
| ATOM | 1357 | CA | GLY | A | 201 | −29.440 | 6.228 | 54.676 | 1.00 | 73.23 | A |
| ATOM | 1358 | C | GLY | A | 201 | −30.722 | 5.428 | 54.498 | 1.00 | 71.08 | A |
| ATOM | 1359 | O | GLY | A | 201 | −30.720 | 4.205 | 54.615 | 1.00 | 70.65 | A |
| ATOM | 1360 | N | SER | A | 202 | −31.828 | 6.103 | 54.232 | 1.00 | 68.97 | A |
| ATOM | 1361 | CA | SER | A | 202 | −33.081 | 5.400 | 54.057 | 1.00 | 67.80 | A |
| ATOM | 1362 | CB | SER | A | 202 | −34.061 | 5.770 | 55.160 | 1.00 | 68.77 | A |
| ATOM | 1363 | OG | SER | A | 202 | −34.504 | 7.105 | 55.003 | 1.00 | 71.14 | A |
| ATOM | 1364 | C | SER | A | 202 | −33.654 | 5.787 | 52.714 | 1.00 | 67.54 | A |
| ATOM | 1365 | O | SER | A | 202 | −34.021 | 6.940 | 52.490 | 1.00 | 67.36 | A |
| ATOM | 1366 | N | VAL | A | 203 | −33.719 | 4.808 | 51.821 | 1.00 | 66.29 | A |
| ATOM | 1367 | CA | VAL | A | 203 | −34.228 | 5.007 | 50.477 | 1.00 | 64.09 | A |
| ATOM | 1368 | CB | VAL | A | 203 | −34.515 | 3.666 | 49.821 | 1.00 | 63.83 | A |
| ATOM | 1369 | CG1 | VAL | A | 203 | −35.014 | 3.883 | 48.412 | 1.00 | 63.72 | A |
| ATOM | 1370 | CG2 | VAL | A | 203 | −33.266 | 2.815 | 49.841 | 1.00 | 61.84 | A |
| ATOM | 1371 | C | VAL | A | 203 | −35.487 | 5.864 | 50.408 | 1.00 | 63.52 | A |
| ATOM | 1372 | O | VAL | A | 203 | −35.473 | 6.924 | 49.794 | 1.00 | 63.71 | A |
| ATOM | 1373 | N | ILE | A | 204 | −36.569 | 5.398 | 51.028 | 1.00 | 62.59 | A |
| ATOM | 1374 | CA | ILE | A | 204 | −37.845 | 6.119 | 51.025 | 1.00 | 62.57 | A |
| ATOM | 1375 | CB | ILE | A | 204 | −38.844 | 5.537 | 52.067 | 1.00 | 62.03 | A |
| ATOM | 1376 | CG2 | ILE | A | 204 | −39.880 | 6.589 | 52.454 | 1.00 | 61.08 | A |
| ATOM | 1377 | CG1 | ILE | A | 204 | −39.549 | 4.299 | 51.503 | 1.00 | 60.70 | A |
| ATOM | 1378 | CD1 | ILE | A | 204 | −40.504 | 4.582 | 50.362 | 1.00 | 58.51 | A |
| ATOM | 1379 | C | ILE | A | 204 | −37.705 | 7.603 | 51.308 | 1.00 | 64.09 | A |
| ATOM | 1380 | O | ILE | A | 204 | −38.298 | 8.433 | 50.624 | 1.00 | 65.20 | A |
| ATOM | 1381 | N | LEU | A | 205 | −36.934 | 7.936 | 52.333 | 1.00 | 65.50 | A |
| ATOM | 1382 | CA | LEU | A | 205 | −36.732 | 9.328 | 52.701 | 1.00 | 66.35 | A |
| ATOM | 1383 | CB | LEU | A | 205 | −35.953 | 9.406 | 54.004 | 1.00 | 65.95 | A |
| ATOM | 1384 | CG | LEU | A | 205 | −36.470 | 10.463 | 54.964 | 1.00 | 66.51 | A |
| ATOM | 1385 | CD1 | LEU | A | 205 | −37.950 | 10.213 | 55.271 | 1.00 | 66.15 | A |
| ATOM | 1386 | CD2 | LEU | A | 205 | −35.630 | 10.412 | 56.224 | 1.00 | 67.48 | A |
| ATOM | 1387 | C | LEU | A | 205 | −35.982 | 10.067 | 51.602 | 1.00 | 67.36 | A |
| ATOM | 1388 | O | LEU | A | 205 | −36.413 | 11.121 | 51.155 | 1.00 | 66.96 | A |
| ATOM | 1389 | N | GLY | A | 206 | −34.858 | 9.500 | 51.170 | 1.00 | 69.84 | A |
| ATOM | 1390 | CA | GLY | A | 206 | −34.067 | 10.109 | 50.116 | 1.00 | 71.82 | A |
| ATOM | 1391 | C | GLY | A | 206 | −34.879 | 10.398 | 48.866 | 1.00 | 73.75 | A |
| ATOM | 1392 | O | GLY | A | 206 | −34.962 | 11.546 | 48.427 | 1.00 | 74.56 | A |
| ATOM | 1393 | N | ALA | A | 207 | −35.479 | 9.363 | 48.285 | 1.00 | 75.29 | A |
| ATOM | 1394 | CA | ALA | A | 207 | −36.281 | 9.539 | 47.079 | 1.00 | 76.82 | A |
| ATOM | 1395 | CB | ALA | A | 207 | −37.123 | 8.295 | 46.817 | 1.00 | 76.91 | A |
| ATOM | 1396 | C | ALA | A | 207 | −37.180 | 10.758 | 47.241 | 1.00 | 77.73 | A |
| ATOM | 1397 | O | ALA | A | 207 | −37.247 | 11.603 | 46.355 | 1.00 | 77.84 | A |
| ATOM | 1398 | N | ILE | A | 208 | −37.855 | 10.845 | 48.385 | 1.00 | 79.24 | A |
| ATOM | 1399 | CA | ILE | A | 208 | −38.755 | 11.963 | 48.687 | 1.00 | 80.73 | A |
| ATOM | 1400 | CB | ILE | A | 208 | −39.441 | 11.785 | 50.077 | 1.00 | 80.51 | A |
| ATOM | 1401 | CG2 | ILE | A | 208 | −39.995 | 13.119 | 50.570 | 1.00 | 79.25 | A |
| ATOM | 1402 | CG1 | ILE | A | 208 | −40.545 | 10.727 | 49.988 | 1.00 | 80.51 | A |
| ATOM | 1403 | CD1 | ILE | A | 208 | −41.645 | 11.051 | 48.976 | 1.00 | 81.04 | A |
| ATOM | 1404 | C | ILE | A | 208 | −38.018 | 13.299 | 48.689 | 1.00 | 81.58 | A |
| ATOM | 1405 | O | ILE | A | 208 | −38.470 | 14.267 | 48.074 | 1.00 | 81.56 | A |
| ATOM | 1406 | N | ARG | A | 209 | −36.894 | 13.337 | 49.403 | 1.00 | 81.98 | A |
| ATOM | 1407 | CA | ARG | A | 209 | −36.072 | 14.534 | 49.509 | 1.00 | 82.11 | A |
| ATOM | 1408 | CB | ARG | A | 209 | −34.697 | 14.184 | 50.090 | 1.00 | 80.96 | A |
| ATOM | 1409 | CG | ARG | A | 209 | −34.107 | 15.243 | 51.019 | 1.00 | 80.28 | A |
| ATOM | 1410 | CD | ARG | A | 209 | −34.311 | 16.652 | 50.477 | 1.00 | 80.15 | A |
| ATOM | 1411 | NE | ARG | A | 209 | −33.866 | 17.693 | 51.403 | 1.00 | 82.03 | A |
| ATOM | 1412 | CZ | ARG | A | 209 | −34.174 | 17.744 | 52.700 | 1.00 | 82.28 | A |
| ATOM | 1413 | NH1 | ARG | A | 209 | −34.932 | 16.803 | 53.250 | 1.00 | 82.85 | A |
| ATOM | 1414 | NH2 | ARG | A | 209 | −33.728 | 18.747 | 53.452 | 1.00 | 81.82 | A |
| ATOM | 1415 | C | ARG | A | 209 | −35.896 | 15.137 | 48.123 | 1.00 | 83.06 | A |
| ATOM | 1416 | O | ARG | A | 209 | −36.351 | 16.251 | 47.850 | 1.00 | 83.03 | A |
| ATOM | 1417 | N | SER | A | 210 | −35.251 | 14.375 | 47.247 | 1.00 | 83.96 | A |
| ATOM | 1418 | CA | SER | A | 210 | −34.987 | 14.805 | 45.879 | 1.00 | 85.24 | A |
| ATOM | 1419 | CB | SER | A | 210 | −34.144 | 13.747 | 45.176 | 1.00 | 85.59 | A |
| ATOM | 1420 | OG | SER | A | 210 | −32.953 | 13.517 | 45.903 | 1.00 | 87.55 | A |
| ATOM | 1421 | C | SER | A | 210 | −36.223 | 15.107 | 45.032 | 1.00 | 85.42 | A |
| ATOM | 1422 | O | SER | A | 210 | −36.717 | 16.236 | 45.015 | 1.00 | 85.30 | A |
| ATOM | 1423 | N | ALA | A | 211 | −36.708 | 14.088 | 44.329 | 1.00 | 85.85 | A |
| ATOM | 1424 | CA | ALA | A | 211 | −37.863 | 14.208 | 43.438 | 1.00 | 86.43 | A |
| ATOM | 1425 | CB | ALA | A | 211 | −38.085 | 12.872 | 42.714 | 1.00 | 86.13 | A |
| ATOM | 1426 | C | ALA | A | 211 | −39.180 | 14.672 | 44.064 | 1.00 | 86.40 | A |
| ATOM | 1427 | O | ALA | A | 211 | −40.235 | 14.141 | 43.724 | 1.00 | 86.26 | A |
| ATOM | 1428 | N | ALA | A | 212 | −39.131 | 15.666 | 44.950 | 1.00 | 87.02 | A |
| ATOM | 1429 | CA | ALA | A | 212 | −40.351 | 16.166 | 45.595 | .00 | 87.20 | A |
| ATOM | 1430 | CB | ALA | A | 212 | −40.944 | 15.073 | 46.494 | 1.00 | 86.67 | A |
| ATOM | 1431 | C | ALA | A | 212 | −40.181 | 17.464 | 46.401 | 1.00 | 87.76 | A |
| ATOM | 1432 | O | ALA | A | 212 | −41.009 | 17.776 | 47.265 | 1.00 | 87.23 | A |
| ATOM | 1433 | N | ALA | A | 213 | −39.118 | 18.216 | 46.116 | 1.00 | 87.91 | A |
| ATOM | 1434 | CA | ALA | A | 213 | −38.850 | 19.470 | 46.815 | 1.00 | 87.67 | A |
| ATOM | 1435 | CB | ALA | A | 213 | −38.472 | 19.189 | 48.262 | 1.00 | 87.74 | A |
| ATOM | 1436 | C | ALA | A | 213 | −37.731 | 20.241 | 46.130 | 1.00 | 87.87 | A |
| ATOM | 1437 | O | ALA | A | 213 | −36.553 | 19.948 | 46.333 | 1.00 | 88.51 | A |
| ATOM | 1438 | N | ALA | A | 224 | −27.138 | 20.797 | 29.818 | .50 | 73.06 | A |
| ATOM | 1439 | CA | ALA | A | 224 | −28.003 | 19.680 | 29.456 | .50 | 72.95 | A |
| ATOM | 1440 | CB | ALA | A | 224 | −28.273 | 18.801 | 30.681 | .50 | 72.80 | A |
| ATOM | 1441 | C | ALA | A | 224 | −29.314 | 20.200 | 28.885 | .50 | 72.70 | A |
| ATOM | 1442 | O | ALA | A | 224 | −29.994 | 19.496 | 28.135 | .50 | 72.83 | A |
| ATOM | 1443 | N | ALA | A | 225 | −29.657 | 21.435 | 29.246 | .50 | 72.39 | A |
| ATOM | 1444 | CA | ALA | A | 225 | −30.886 | 22.073 | 28.782 | .50 | 72.88 | A |
| ATOM | 1445 | CB | ALA | A | 225 | −30.899 | 23.543 | 29.194 | .50 | 71.61 | A |
| ATOM | 1446 | C | ALA | A | 225 | −31.041 | 21.946 | 27.266 | .50 | 73.89 | A |
| ATOM | 1447 | O | ALA | A | 225 | −30.146 | 22.319 | 26.499 | .50 | 73.39 | A |
| ATOM | 1448 | N | ALA | A | 226 | −32.187 | 21.409 | 26.846 | .50 | 75.01 | A |
| ATOM | 1449 | CA | ALA | A | 226 | −32.489 | 21.206 | 25.432 | .50 | 75.80 | A |
| ATOM | 1450 | CB | ALA | A | 226 | −32.502 | 19.711 | 25.113 | .50 | 75.25 | A |
| ATOM | 1451 | C | ALA | A | 226 | −33.835 | 21.829 | 25.069 | .50 | 76.51 | A |
| ATOM | 1452 | O | ALA | A | 226 | −34.241 | 22.837 | 25.653 | .50 | 76.21 | A |
| ATOM | 1453 | N | ALA | A | 227 | −34.518 | 21.220 | 24.103 | .50 | 77.48 | A |
| ATOM | 1454 | CA | ALA | A | 227 | −35.824 | 21.699 | 23.649 | .50 | 78.46 | A |
| ATOM | 1455 | CB | ALA | A | 227 | −36.009 | 21.390 | 22.155 | .50 | 77.48 | A |
| ATOM | 1456 | C | ALA | A | 227 | −36.959 | 21.065 | 24.460 | .50 | 78.98 | A |
| ATOM | 1457 | O | ALA | A | 227 | −37.037 | 21.235 | 25.681 | .50 | 78.83 | A |
| ATOM | 1458 | N | ALA | A | 228 | −37.834 | 20.340 | 23.766 | .50 | 79.11 | A |
| ATOM | 1459 | CA | ALA | A | 228 | −38.966 | 19.669 | 24.392 | .50 | 79.12 | A |
| ATOM | 1460 | CB | ALA | A | 228 | −38.473 | 18.503 | 25.250 | .50 | 78.73 | A |
| ATOM | 1461 | C | ALA | A | 228 | −39.827 | 20.612 | 25.230 | .50 | 79.39 | A |
| ATOM | 1462 | O | ALA | A | 228 | −39.661 | 21.833 | 25.194 | .50 | 78.77 | A |
| ATOM | 1463 | N | ALA | A | 229 | −40.756 | 20.025 | 25.975 | .50 | 80.01 | A |
| ATOM | 1464 | CA | ALA | A | 229 | −41.664 | 20.772 | 26.835 | .50 | 81.30 | A |
| ATOM | 1465 | CB | ALA | A | 229 | −42.642 | 21.583 | 25.988 | .50 | 80.67 | A |
| ATOM | 1466 | C | ALA | A | 229 | −42.419 | 19.762 | 27.699 | .50 | 82.42 | A |
| ATOM | 1467 | O | ALA | A | 229 | −43.642 | 19.856 | 27.871 | .50 | 82.33 | A |
| ATOM | 1468 | N | ALA | A | 230 | −41.672 | 18.794 | 28.233 | .50 | 83.00 | A |
| ATOM | 1469 | CA | ALA | A | 230 | −42.233 | 17.739 | 29.069 | .50 | 82.75 | A |
| ATOM | 1470 | CB | ALA | A | 230 | −42.784 | 18.326 | 30.362 | .50 | 82.42 | A |
| ATOM | 1471 | C | ALA | A | 230 | −43.338 | 17.047 | 28.290 | .50 | 83.06 | A |
| ATOM | 1472 | O | ALA | A | 230 | −44.175 | 16.351 | 28.861 | .50 | 83.03 | A |
| ATOM | 1473 | N | ALA | A | 231 | −43.328 | 17.249 | 26.975 | 1.00 | 83.94 | A |
| ATOM | 1474 | CA | ALA | A | 231 | −44.325 | 16.660 | 26.085 | 1.00 | 84.89 | A |
| ATOM | 1475 | CB | ALA | A | 231 | −44.251 | 17.321 | 24.700 | 1.00 | 84.01 | A |
| ATOM | 1476 | C | ALA | A | 231 | −44.133 | 15.146 | 25.961 | 1.00 | 85.05 | A |
| ATOM | 1477 | O | ALA | A | 231 | −43.089 | 14.673 | 25.502 | 1.00 | 85.19 | A |
| ATOM | 1478 | N | ALA | A | 232 | −45.154 | 14.400 | 26.380 | 1.00 | 85.34 | A |
| ATOM | 1479 | CA | ALA | A | 232 | −45.149 | 12.938 | 26.340 | 1.00 | 84.53 | A |
| ATOM | 1480 | CB | ALA | A | 232 | −45.437 | 12.451 | 24.912 | 1.00 | 84.18 | A |
| ATOM | 1481 | C | ALA | A | 232 | −43.814 | 12.389 | 26.840 | 1.00 | 83.93 | A |
| ATOM | 1482 | O | ALA | A | 232 | −42.990 | 11.909 | 26.061 | 1.00 | 85.22 | A |
| ATOM | 1483 | N | ALA | A | 233 | −43.599 | 12.470 | 28.148 | 1.00 | 82.68 | A |
| ATOM | 1484 | CA | ALA | A | 233 | −42.358 | 11.985 | 28.735 | 1.00 | 80.84 | A |
| ATOM | 1485 | CB | ALA | A | 233 | −42.253 | 12.446 | 30.195 | 1.00 | 80.93 | A |
| ATOM | 1486 | C | ALA | A | 233 | −42.271 | 10.460 | 28.655 | 1.00 | 79.29 | A |
| ATOM | 1487 | O | ALA | A | 233 | −41.194 | 9.885 | 28.856 | 1.00 | 80.96 | A |
| ATOM | 1488 | N | GLY | A | 234 | −43.400 | 9.817 | 28.349 | 1.00 | 75.37 | A |
| ATOM | 1489 | CA | GLY | A | 234 | −43.439 | 8.367 | 28.266 | 1.00 | 71.08 | A |
| ATOM | 1490 | C | GLY | A | 234 | −42.455 | 7.776 | 27.277 | 1.00 | 68.67 | A |
| ATOM | 1491 | O | GLY | A | 234 | −41.659 | 8.498 | 26.668 | 1.00 | 69.64 | A |
| ATOM | 1492 | N | SER | A | 235 | −42.492 | 6.457 | 27.121 | 1.00 | 64.72 | A |
| ATOM | 1493 | CA | SER | A | 235 | −41.605 | 5.800 | 26.177 | 1.00 | 61.31 | A |
| ATOM | 1494 | CB | SER | A | 235 | −41.447 | 4.314 | 26.523 | 1.00 | 61.14 | A |
| ATOM | 1495 | OG | SER | A | 235 | −42.616 | 3.573 | 26.230 | 1.00 | 60.70 | A |
| ATOM | 1496 | C | SER | A | 235 | −42.230 | 5.973 | 24.790 | 1.00 | 60.47 | A |
| ATOM | 1497 | O | SER | A | 235 | −43.266 | 6.628 | 24.653 | 1.00 | 61.47 | A |
| ATOM | 1498 | N | PHE | A | 236 | −41.609 | 5.401 | 23.762 | 1.00 | 57.28 | A |
| ATOM | 1499 | CA | PHE | A | 236 | −42.131 | 5.531 | 22.408 | 1.00 | 52.78 | A |
| ATOM | 1500 | CB | PHE | A | 236 | −42.148 | 7.005 | 21.995 | 1.00 | 49.10 | A |
| ATOM | 1501 | CG | PHE | A | 236 | −40.781 | 7.608 | 21.859 | 1.00 | 44.98 | A |
| ATOM | 1502 | CD1 | PHE | A | 236 | −39.963 | 7.277 | 20.790 | 1.00 | 44.07 | A |
| ATOM | 1503 | CD2 | PHE | A | 236 | −40.288 | 8.461 | 22.834 | 1.00 | 44.28 | A |
| ATOM | 1504 | CE1 | PHE | A | 236 | −38.682 | 7.778 | 20.702 | 1.00 | 42.76 | A |
| ATOM | 1505 | CE2 | PHE | A | 236 | −38.994 | 8.969 | 22.754 | 1.00 | 40.96 | A |
| ATOM | 1506 | CZ | PHE | A | 236 | −38.197 | 8.625 | 21.691 | 1.00 | 42.11 | A |
| ATOM | 1507 | C | PHE | A | 236 | −41.268 | 4.752 | 21.437 | 1.00 | 51.51 | A |
| ATOM | 1508 | O | PHE | A | 236 | −40.286 | 4.142 | 21.825 | 1.00 | 52.57 | A |
| ATOM | 1509 | N | SER | A | 237 | −41.637 | 4.801 | 20.164 | 1.00 | 50.23 | A |
| ATOM | 1510 | CA | SER | A | 237 | −40.905 | 4.117 | 19.112 | 1.00 | 47.37 | A |
| ATOM | 1511 | CB | SER | A | 237 | −41.552 | 2.753 | 18.853 | 1.00 | 45.96 | A |
| ATOM | 1512 | OG | SER | A | 237 | −40.872 | 2.035 | 17.851 | 1.00 | 41.87 | A |
| ATOM | 1513 | C | SER | A | 237 | −40.973 | 5.008 | 17.868 | 1.00 | 46.77 | A |
| ATOM | 1514 | O | SER | A | 237 | −41.077 | 6.224 | 17.984 | 1.00 | 44.08 | A |
| ATOM | 1515 | N | PHE | A | 238 | −40.888 | 4.401 | 16.689 | 1.00 | 47.90 | A |
| ATOM | 1516 | CA | PHE | A | 238 | −40.973 | 5.124 | 15.423 | 1.00 | 48.39 | A |
| ATOM | 1517 | CB | PHE | A | 238 | −39.587 | 5.391 | 14.834 | 1.00 | 47.01 | A |
| ATOM | 1518 | CG | PHE | A | 238 | −38.685 | 6.190 | 15.725 | 1.00 | 45.95 | A |
| ATOM | 1519 | CD1 | PHE | A | 238 | −37.802 | 5.553 | 16.586 | 1.00 | 43.07 | A |
| ATOM | 1520 | CD2 | PHE | A | 238 | −38.732 | 7.584 | 15.717 | 1.00 | 45.63 | A |
| ATOM | 1521 | CE1 | PHE | A | 238 | −36.990 | 6.280 | 17.421 | 1.00 | 41.89 | A |
| ATOM | 1522 | CE2 | PHE | A | 238 | −37.920 | 8.325 | 16.557 | 1.00 | 45.42 | A |
| ATOM | 1523 | CZ | PHE | A | 238 | −37.044 | 7.665 | 17.416 | 1.00 | 45.74 | A |
| ATOM | 1524 | C | PHE | A | 238 | −41.766 | 4.273 | 14.437 | 1.00 | 50.14 | A |
| ATOM | 1525 | O | PHE | A | 238 | −41.722 | 3.038 | 14.489 | 1.00 | 50.51 | A |
| ATOM | 1526 | N | ALA | A | 239 | −42.493 | 4.923 | 13.537 | 1.00 | 51.76 | A |
| ATOM | 1527 | CA | ALA | A | 239 | −43.277 | 4.186 | 12.554 | 1.00 | 52.99 | A |
| ATOM | 1528 | CB | ALA | A | 239 | −43.989 | 5.164 | 11.610 | 1.00 | 53.28 | A |
| ATOM | 1529 | C | ALA | A | 239 | −42.334 | 3.263 | 11.775 | 1.00 | 54.16 | A |
| ATOM | 1530 | O | ALA | A | 239 | −41.359 | 3.721 | 11.168 | 1.00 | 54.00 | A |
| ATOM | 1531 | N | GLY | A | 240 | −42.618 | 1.963 | 11.814 | 1.00 | 55.32 | A |
| ATOM | 1532 | CA | GLY | A | 240 | −41.784 | .995 | 11.124 | 1.00 | 54.98 | A |
| ATOM | 1533 | C | GLY | A | 240 | −40.886 | .208 | 12.066 | 1.00 | 56.75 | A |
| ATOM | 1534 | O | GLY | A | 240 | −40.347 | −.827 | 11.680 | 1.00 | 58.11 | A |
| ATOM | 1535 | N | GLY | A | 241 | −40.720 | .681 | 13.300 | 1.00 | 55.92 | A |
| ATOM | 1536 | CA | GLY | A | 241 | −39.868 | −.023 | 14.238 | 1.00 | 55.24 | A |
| ATOM | 1537 | C | GLY | A | 241 | −38.742 | .838 | 14.786 | 1.00 | 55.94 | A |
| ATOM | 1538 | O | GLY | A | 241 | −38.513 | 1.963 | 14.341 | 1.00 | 54.46 | A |
| ATOM | 1539 | N | MSE | A | 242 | −38.025 | .306 | 15.765 | 1.00 | 56.05 | A |
| ATOM | 1540 | CA | MSE | A | 242 | −36.942 | 1.046 | 16.372 | 1.00 | 55.41 | A |
| ATOM | 1541 | CB | MSE | A | 242 | −36.404 | .290 | 17.589 | 1.00 | 56.50 | A |
| ATOM | 1542 | CG | MSE | A | 242 | −37.219 | .477 | 18.855 | 1.00 | 57.58 | A |
| ATOM | 1543 | SE | MSE | A | 242 | −37.281 | 2.335 | 19.450 | 1.00 | 63.46 | A |
| ATOM | 1544 | CE | MSE | A | 242 | −35.453 | 2.473 | 20.083 | 1.00 | 60.51 | A |
| ATOM | 1545 | C | MSE | A | 242 | −35.824 | 1.295 | 15.390 | 1.00 | 55.51 | A |
| ATOM | 1546 | O | MSE | A | 242 | −35.066 | 2.246 | 15.548 | 1.00 | 54.44 | A |
| ATOM | 1547 | N | GLN | A | 243 | −35.731 | .454 | 14.365 | 1.00 | 56.39 | A |
| ATOM | 1548 | CA | GLN | A | 243 | −34.655 | .585 | 13.391 | 1.00 | 57.64 | A |
| ATOM | 1549 | CB | GLN | A | 243 | −34.640 | −.614 | 12.428 | 1.00 | 59.46 | A |
| ATOM | 1550 | CG | GLN | A | 243 | −33.215 | −.978 | 11.964 | 1.00 | 61.76 | A |
| ATOM | 1551 | CD | GLN | A | 243 | −33.160 | −2.043 | 10.882 | 1.00 | 61.93 | A |
| ATOM | 1552 | OE1 | GLN | A | 243 | −33.657 | −3.167 | 11.049 | 1.00 | 60.05 | A |
| ATOM | 1553 | NE2 | GLN | A | 243 | −32.537 | −1.693 | 9.762 | 1.00 | 62.19 | A |
| ATOM | 1554 | C | GLN | A | 243 | −34.691 | 1.888 | 12.604 | 1.00 | 57.35 | A |
| ATOM | 1555 | O | GLN | A | 243 | −33.675 | 2.329 | 12.065 | 1.00 | 57.21 | A |
| ATOM | 1556 | N | THR | A | 244 | −35.860 | 2.514 | 12.554 | 1.00 | 58.01 | A |
| ATOM | 1557 | CA | THR | A | 244 | −36.020 | 3.768 | 11.830 | 1.00 | 56.63 | A |
| ATOM | 1558 | CB | THR | A | 244 | −37.428 | 4.338 | 12.012 | 1.00 | 56.36 | A |
| ATOM | 1559 | OG1 | THR | A | 244 | −38.380 | 3.452 | 11.403 | 1.00 | 57.44 | A |
| ATOM | 1560 | CG2 | THR | A | 244 | −37.527 | 5.718 | 11.380 | 1.00 | 56.62 | A |
| ATOM | 1561 | C | THR | A | 244 | −35.014 | 4.806 | 12.275 | 1.00 | 55.83 | A |
| ATOM | 1562 | O | THR | A | 244 | −34.521 | 5.571 | 11.460 | 1.00 | 56.94 | A |
| ATOM | 1563 | N | LEU | A | 245 | −34.714 | 4.832 | 13.568 | 1.00 | 55.53 | A |
| ATOM | 1564 | CA | LEU | A | 245 | −33.744 | 5.778 | 14.119 | 1.00 | 55.88 | A |
| ATOM | 1565 | CB | LEU | A | 245 | −33.687 | 5.622 | 15.643 | 1.00 | 54.34 | A |
| ATOM | 1566 | CG | LEU | A | 245 | −32.531 | 6.275 | 16.416 | 1.00 | 54.58 | A |
| ATOM | 1567 | CD1 | LEU | A | 245 | −32.455 | 7.773 | 16.139 | 1.00 | 52.75 | A |
| ATOM | 1568 | CD2 | LEU | A | 245 | −32.736 | 6.015 | 17.906 | 1.00 | 54.73 | A |
| ATOM | 1569 | C | LEU | A | 245 | −32.350 | 5.555 | 13.517 | 1.00 | 56.39 | A |
| ATOM | 1570 | O | LEU | A | 245 | −31.699 | 6.482 | 13.038 | 1.00 | 55.27 | A |
| ATOM | 1571 | N | THR | A | 246 | −31.909 | 4.306 | 13.557 | 1.00 | 58.43 | A |
| ATOM | 1572 | CA | THR | A | 246 | −30.622 | 3.891 | 13.030 | 1.00 | 59.63 | A |
| ATOM | 1573 | CB | THR | A | 246 | −30.450 | 2.405 | 13.276 | 1.00 | 58.63 | A |
| ATOM | 1574 | OG1 | THR | A | 246 | −29.714 | 2.234 | 14.486 | 1.00 | 58.60 | A |
| ATOM | 1575 | CG2 | THR | A | 246 | −29.745 | 1.729 | 12.110 | 1.00 | 60.77 | A |
| ATOM | 1576 | C | THR | A | 246 | −30.467 | 4.200 | 11.538 | 1.00 | 61.80 | A |
| ATOM | 1577 | O | THR | A | 246 | −29.401 | 4.642 | 11.095 | 1.00 | 62.73 | A |
| ATOM | 1578 | N | ASP | A | 247 | −31.530 | 3.959 | 10.772 | 1.00 | 62.36 | A |
| ATOM | 1579 | CA | ASP | A | 247 | −31.549 | 4.230 | 9.336 | 1.00 | 62.33 | A |
| ATOM | 1580 | CB | ASP | A | 247 | −32.870 | 3.761 | 8.745 | 1.00 | 63.74 | A |
| ATOM | 1581 | CG | ASP | A | 247 | −32.995 | 2.259 | 8.735 | 1.00 | 65.46 | A |
| ATOM | 1582 | OD1 | ASP | A | 247 | −32.160 | 1.582 | 9.378 | 1.00 | 68.06 | A |
| ATOM | 1583 | OD2 | ASP | A | 247 | −33.932 | 1.757 | 8.086 | 1.00 | 64.74 | A |
| ATOM | 1584 | C | ASP | A | 247 | −31.366 | 5.718 | 9.035 | 1.00 | 62.47 | A |
| ATOM | 1585 | O | ASP | A | 247 | −30.672 | 6.085 | 8.094 | 1.00 | 63.22 | A |
| ATOM | 1586 | N | ALA | A | 248 | −32.003 | 6.569 | 9.829 | 1.00 | 61.81 | A |
| ATOM | 1587 | CA | ALA | A | 248 | −31.889 | 8.006 | 9.651 | 1.00 | 61.70 | A |
| ATOM | 1588 | CB | ALA | A | 248 | −32.706 | 8.722 | 10.711 | 1.00 | 62.83 | A |
| ATOM | 1589 | C | ALA | A | 248 | −30.431 | 8.451 | 9.734 | 1.00 | 62.09 | A |
| ATOM | 1590 | O | ALA | A | 248 | −30.003 | 9.330 | 8.988 | 1.00 | 62.23 | A |
| ATOM | 1591 | N | ILE | A | 249 | −29.670 | 7.849 | 10.645 | 1.00 | 61.72 | A |
| ATOM | 1592 | CA | ILE | A | 249 | −28.269 | 8.210 | 10.806 | 1.00 | 61.70 | A |
| ATOM | 1593 | CB | ILE | A | 249 | −27.673 | 7.627 | 12.107 | 1.00 | 61.12 | A |
| ATOM | 1594 | CG2 | ILE | A | 249 | −26.204 | 8.023 | 12.237 | 1.00 | 58.39 | A |
| ATOM | 1595 | CG1 | ILE | A | 249 | −28.451 | 8.155 | 13.315 | 1.00 | 60.88 | A |
| ATOM | 1596 | CD1 | ILE | A | 249 | −28.033 | 7.558 | 14.642 | 1.00 | 61.28 | A |
| ATOM | 1597 | C | ILE | A | 249 | −27.470 | 7.702 | 9.625 | 1.00 | 62.92 | A |
| ATOM | 1598 | O | ILE | A | 249 | −26.538 | 8.354 | 9.166 | 1.00 | 63.03 | A |
| ATOM | 1599 | N | CYS | A | 250 | −27.849 | 6.537 | 9.123 | 1.00 | 64.67 | A |
| ATOM | 1600 | CA | CYS | A | 250 | −27.159 | 5.938 | 7.993 | 1.00 | 67.07 | A |
| ATOM | 1601 | CB | CYS | A | 250 | −27.570 | 4.477 | 7.857 | 1.00 | 66.86 | A |
| ATOM | 1602 | SG | CYS | A | 250 | −26.733 | 3.406 | 9.029 | 1.00 | 68.00 | A |
| ATOM | 1603 | C | CYS | A | 250 | −27.353 | 6.639 | 6.652 | 1.00 | 69.00 | A |
| ATOM | 1604 | O | CYS | A | 250 | −26.544 | 6.459 | 5.741 | 1.00 | 69.96 | A |
| ATOM | 1605 | N | LYS | A | 251 | −28.422 | 7.421 | 6.521 | 1.00 | 70.74 | A |
| ATOM | 1606 | CA | LYS | A | 251 | −28.690 | 8.129 | 5.274 | 1.00 | 71.15 | A |
| ATOM | 1607 | CB | LYS | A | 251 | −30.171 | 8.520 | 5.174 | 1.00 | 72.31 | A |
| ATOM | 1608 | CG | LYS | A | 251 | −31.154 | 7.346 | 5.101 | 1.00 | 72.63 | A |
| ATOM | 1609 | CD | LYS | A | 251 | −30.873 | 6.423 | 3.917 | 1.00 | 74.21 | A |
| ATOM | 1610 | CE | LYS | A | 251 | −29.758 | 5.429 | 4.219 | 1.00 | 74.30 | A |
| ATOM | 1611 | NZ | LYS | A | 251 | −29.470 | 4.539 | 3.059 | 1.00 | 75.05 | A |
| ATOM | 1612 | C | LYS | A | 251 | −27.824 | 9.367 | 5.166 | 1.00 | 70.84 | A |
| ATOM | 1613 | O | LYS | A | 251 | −27.932 | 10.119 | 4.208 | 1.00 | 72.01 | A |
| ATOM | 1614 | N | ASP | A | 252 | −26.967 | 9.576 | 6.158 | 1.00 | 71.24 | A |
| ATOM | 1615 | CA | ASP | A | 252 | −26.062 | 10.723 | 6.162 | 1.00 | 71.90 | A |
| ATOM | 1616 | CB | ASP | A | 252 | −26.191 | 11.522 | 7.464 | 1.00 | 72.92 | A |
| ATOM | 1617 | CG | ASP | A | 252 | −27.569 | 12.124 | 7.648 | 1.00 | 74.69 | A |
| ATOM | 1618 | OD1 | ASP | A | 252 | −28.546 | 11.342 | 7.706 | 1.00 | 76.34 | A |
| ATOM | 1619 | OD2 | ASP | A | 252 | −27.674 | 13.372 | 7.738 | 1.00 | 74.87 | A |
| ATOM | 1620 | C | ASP | A | 252 | −24.636 | 10.214 | 6.034 | 1.00 | 71.36 | A |
| ATOM | 1621 | O | ASP | A | 252 | −23.675 | 10.969 | 6.172 | 1.00 | 71.25 | A |
| ATOM | 1622 | N | LEU | A | 253 | −24.508 | 8.920 | 5.779 | 1.00 | 70.88 | A |
| ATOM | 1623 | CA | LEU | A | 253 | −23.202 | 8.303 | 5.643 | 1.00 | 70.63 | A |
| ATOM | 1624 | CB | LEU | A | 253 | −23.012 | 7.235 | 6.730 | 1.00 | 70.65 | A |
| ATOM | 1625 | CG | LEU | A | 253 | −22.979 | 7.697 | 8.195 | 1.00 | 69.91 | A |
| ATOM | 1626 | CD1 | LEU | A | 253 | −22.838 | 6.490 | 9.100 | 1.00 | 68.58 | A |
| ATOM | 1627 | CD2 | LEU | A | 253 | −21.823 | 8.656 | 8.426 | 1.00 | 68.83 | A |
| ATOM | 1628 | C | LEU | A | 253 | −23.053 | 7.684 | 4.262 | 1.00 | 70.35 | A |
| ATOM | 1629 | O | LEU | A | 253 | −23.941 | 6.975 | 3.790 | 1.00 | 69.55 | A |
| ATOM | 1630 | N | ARG | A | 254 | −21.924 | 7.967 | 3.618 | 1.00 | 70.96 | A |
| ATOM | 1631 | CA | ARG | A | 254 | −21.644 | 7.447 | 2.284 | 1.00 | 71.48 | A |
| ATOM | 1632 | CB | ARG | A | 254 | −20.446 | 8.187 | 1.687 | 1.00 | 70.98 | A |
| ATOM | 1633 | CG | ARG | A | 254 | −20.451 | 9.666 | 2.019 | 1.00 | 72.19 | A |
| ATOM | 1634 | CD | ARG | A | 254 | −19.424 | 10.458 | 1.222 | 1.00 | 74.34 | A |
| ATOM | 1635 | NE | ARG | A | 254 | −19.029 | 11.678 | 1.931 | 1.00 | 77.18 | A |
| ATOM | 1636 | CZ | ARG | A | 254 | −17.922 | 11.796 | 2.669 | 1.00 | 77.75 | A |
| ATOM | 1637 | NH1 | ARG | A | 254 | −17.086 | 10.769 | 2.787 | 1.00 | 76.14 | A |
| ATOM | 1638 | NH2 | ARG | A | 254 | −17.668 | 12.928 | 3.318 | 1.00 | 76.91 | A |
| ATOM | 1639 | C | ARG | A | 254 | −21.373 | 5.947 | 2.352 | 1.00 | 72.08 | A |
| ATOM | 1640 | O | ARG | A | 254 | −21.216 | 5.391 | 3.436 | 1.00 | 71.82 | A |
| ATOM | 1641 | N | GLU | A | 255 | −21.335 | 5.289 | 1.198 | 1.00 | 73.81 | A |
| ATOM | 1642 | CA | GLU | A | 255 | −21.086 | 3.851 | 1.157 | 1.00 | 76.17 | A |
| ATOM | 1643 | CB | GLU | A | 255 | −21.219 | 3.333 | −.277 | 1.00 | 77.35 | A |
| ATOM | 1644 | CG | GLU | A | 255 | −21.087 | 1.818 | −.405 | 1.00 | 79.94 | A |
| ATOM | 1645 | CD | GLU | A | 255 | −20.505 | 1.398 | −1.745 | 1.00 | 81.38 | A |
| ATOM | 1646 | OE1 | GLU | A | 255 | −21.098 | 1.750 | −2.789 | 1.00 | 82.40 | A |
| ATOM | 1647 | OE2 | GLU | A | 255 | −19.452 | .718 | −1.751 | 1.00 | 81.80 | A |
| ATOM | 1648 | C | GLU | A | 255 | −19.685 | 3.538 | 1.691 | 1.00 | 77.19 | A |
| ATOM | 1649 | O | GLU | A | 255 | −19.404 | 2.420 | 2.138 | 1.00 | 77.49 | A |
| ATOM | 1650 | N | ASP | A | 256 | −18.812 | 4.538 | 1.632 | 1.00 | 77.66 | A |
| ATOM | 1651 | CA | ASP | A | 256 | −17.440 | 4.415 | 2.104 | 1.00 | 78.14 | A |
| ATOM | 1652 | CB | ASP | A | 256 | −16.544 | 5.343 | 1.283 | 1.00 | 79.74 | A |
| ATOM | 1653 | CG | ASP | A | 256 | −17.100 | 6.764 | 1.189 | 1.00 | 81.73 | A |
| ATOM | 1654 | OD1 | ASP | A | 256 | −16.990 | 7.526 | 2.186 | 1.00 | 80.93 | A |
| ATOM | 1655 | OD2 | ASP | A | 256 | −17.658 | 7.106 | .116 | 1.00 | 81.41 | A |
| ATOM | 1656 | C | ASP | A | 256 | −17.340 | 4.754 | 3.595 | 1.00 | 78.03 | A |
| ATOM | 1657 | O | ASP | A | 256 | −16.316 | 4.484 | 4.237 | 1.00 | 78.04 | A |
| ATOM | 1658 | N | GLU | A | 257 | −18.406 | 5.359 | 4.126 | 1.00 | 77.55 | A |
| ATOM | 1659 | CA | GLU | A | 257 | −18.497 | 5.730 | 5.538 | 1.00 | 76.71 | A |
| ATOM | 1660 | CB | GLU | A | 257 | −19.610 | 6.752 | 5.767 | 1.00 | 77.91 | A |
| ATOM | 1661 | CG | GLU | A | 257 | −19.193 | 8.005 | 6.500 | 1.00 | 79.91 | A |
| ATOM | 1662 | CD | GLU | A | 257 | −18.602 | 9.021 | 5.568 | 1.00 | 80.15 | A |
| ATOM | 1663 | OE1 | GLU | A | 257 | −17.616 | 8.678 | 4.881 | 1.00 | 82.94 | A |
| ATOM | 1664 | OE2 | GLU | A | 257 | −19.127 | 10.152 | 5.519 | 1.00 | 78.71 | A |
| ATOM | 1665 | C | GLU | A | 257 | −18.862 | 4.474 | 6.313 | 1.00 | 75.42 | A |
| ATOM | 1666 | O | GLU | A | 257 | −18.216 | 4.127 | 7.299 | 1.00 | 76.62 | A |
| ATOM | 1667 | N | LEU | A | 258 | −19.907 | 3.799 | 5.847 | 1.00 | 72.61 | A |
| ATOM | 1668 | CA | LEU | A | 258 | −20.406 | 2.587 | 6.487 | 1.00 | 70.78 | A |
| ATOM | 1669 | CB | LEU | A | 258 | −21.938 | 2.598 | 6.476 | 1.00 | 70.68 | A |
| ATOM | 1670 | CG | LEU | A | 258 | −22.632 | 1.459 | 7.219 | 1.00 | 68.68 | A |
| ATOM | 1671 | CD1 | LEU | A | 258 | −22.196 | 1.501 | 8.676 | 1.00 | 68.74 | A |
| ATOM | 1672 | CD2 | LEU | A | 258 | −24.135 | 1.591 | 7.092 | 1.00 | 65.03 | A |
| ATOM | 1673 | C | LEU | A | 258 | −19.915 | 1.308 | 5.820 | 1.00 | 69.07 | A |
| ATOM | 1674 | O | LEU | A | 258 | −20.369 | .970 | 4.727 | 1.00 | 68.85 | A |
| ATOM | 1675 | N | ARG | A | 259 | −19.012 | .588 | 6.485 | 1.00 | 67.26 | A |
| ATOM | 1676 | CA | ARG | A | 259 | −18.474 | −.654 | 5.938 | 1.00 | 65.91 | A |
| ATOM | 1677 | CB | ARG | A | 259 | −16.954 | −.674 | 6.066 | 1.00 | 67.76 | A |
| ATOM | 1678 | CG | ARG | A | 259 | −16.283 | .558 | 5.540 | 1.00 | 71.10 | A |
| ATOM | 1679 | CD | ARG | A | 259 | −14.799 | .337 | 5.385 | 1.00 | 74.92 | A |
| ATOM | 1680 | NE | ARG | A | 259 | −14.484 | −.432 | 4.185 | 1.00 | 77.83 | A |
| ATOM | 1681 | CZ | ARG | A | 259 | −13.281 | −.939 | 3.921 | 1.00 | 80.04 | A |
| ATOM | 1682 | NH1 | ARG | A | 259 | −12.283 | −.760 | 4.778 | 1.00 | 79.54 | A |
| ATOM | 1683 | NH2 | ARG | A | 259 | −13.070 | −1.619 | 2.795 | 1.00 | 81.15 | A |
| ATOM | 1684 | C | ARG | A | 259 | −19.055 | −1.882 | 6.628 | 1.00 | 63.94 | A |
| ATOM | 1685 | O | ARG | A | 259 | −18.601 | −2.285 | 7.691 | 1.00 | 62.56 | A |
| ATOM | 1686 | N | LEU | A | 260 | −20.056 | −2.483 | 6.007 | 1.00 | 63.04 | A |
| ATOM | 1687 | CA | LEU | A | 260 | −20.705 | −3.657 | 6.565 | 1.00 | 62.65 | A |
| ATOM | 1688 | CB | LEU | A | 260 | −22.098 | −3.810 | 5.935 | 1.00 | 61.27 | A |
| ATOM | 1689 | CG | LEU | A | 260 | −22.961 | −2.532 | 6.017 | 1.00 | 59.09 | A |
| ATOM | 1690 | CD1 | LEU | A | 260 | −24.163 | −2.621 | 5.098 | 1.00 | 56.19 | A |
| ATOM | 1691 | CD2 | LEU | A | 260 | −23.384 | −2.296 | 7.444 | 1.00 | 55.07 | A |
| ATOM | 1692 | C | LEU | A | 260 | −19.855 | −4.903 | 6.326 | 1.00 | 63.47 | A |
| ATOM | 1693 | O | LEU | A | 260 | −18.916 | −4.880 | 5.534 | 1.00 | 64.12 | A |
| ATOM | 1694 | N | ASN | A | 261 | −20.173 | −5.981 | 7.036 | 1.00 | 64.76 | A |
| ATOM | 1695 | CA | ASN | A | 261 | −19.452 | −7.249 | 6.910 | 1.00 | 65.33 | A |
| ATOM | 1696 | CB | ASN | A | 261 | −19.696 | −7.856 | 5.539 | 1.00 | 65.30 | A |
| ATOM | 1697 | CG | ASN | A | 261 | −21.160 | −8.049 | 5.266 | 1.00 | 68.31 | A |
| ATOM | 1698 | OD1 | ASN | A | 261 | −21.803 | −8.913 | 5.862 | 1.00 | 68.51 | A |
| ATOM | 1699 | ND2 | ASN | A | 261 | −21.714 | −7.223 | 4.379 | 1.00 | 71.48 | A |
| ATOM | 1700 | C | ASN | A | 261 | −17.967 | −7.149 | 7.142 | 1.00 | 65.10 | A |
| ATOM | 1701 | O | ASN | A | 261 | −17.260 | −8.138 | 6.999 | 1.00 | 66.08 | A |
| ATOM | 1702 | N | ALA | A | 262 | −17.489 | −5.963 | 7.502 | 1.00 | 64.52 | A |
| ATOM | 1703 | CA | ALA | A | 262 | −16.065 | −5.780 | 7.744 | 1.00 | 63.89 | A |
| ATOM | 1704 | CB | ALA | A | 262 | −15.589 | −4.469 | 7.109 | 1.00 | 63.36 | A |
| ATOM | 1705 | C | ALA | A | 262 | −15.749 | −5.807 | 9.239 | 1.00 | 63.18 | A |
| ATOM | 1706 | O | ALA | A | 262 | −15.545 | −4.767 | 9.861 | 1.00 | 63.66 | A |
| ATOM | 1707 | N | ARG | A | 263 | −15.713 | −7.006 | 9.812 | 1.00 | 62.57 | A |
| ATOM | 1708 | CA | ARG | A | 263 | −15.411 | −7.170 | 11.233 | 1.00 | 63.29 | A |
| ATOM | 1709 | CB | ARG | A | 263 | −15.745 | −8.590 | 11.683 | 1.00 | 63.59 | A |
| ATOM | 1710 | CG | ARG | A | 263 | −14.729 | −9.175 | 12.667 | 1.00 | 66.17 | A |
| ATOM | 1711 | CD | ARG | A | 263 | −15.241 | −10.442 | 13.357 | 1.00 | 67.91 | A |
| ATOM | 1712 | NE | ARG | A | 263 | −15.914 | −10.139 | 14.620 | 1.00 | 69.52 | A |
| ATOM | 1713 | CZ | ARG | A | 263 | −17.181 | −10.443 | 14.905 | 1.00 | 71.20 | A |
| ATOM | 1714 | NH1 | ARG | A | 263 | −17.941 | −11.073 | 14.015 | 1.00 | 72.00 | A |
| ATOM | 1715 | NH2 | ARG | A | 263 | −17.699 | −10.102 | 16.081 | 1.00 | 71.14 | A |
| ATOM | 1716 | C | ARG | A | 263 | −13.952 | −6.865 | 11.580 | 1.00 | 63.17 | A |
| ATOM | 1717 | O | ARG | A | 263 | −13.037 | −7.353 | 10.928 | 1.00 | 63.33 | A |
| ATOM | 1718 | N | VAL | A | 264 | −13.742 | −6.063 | 12.617 | 1.00 | 62.83 | A |
| ATOM | 1719 | CA | VAL | A | 264 | −12.394 | −5.699 | 13.044 | 1.00 | 62.38 | A |
| ATOM | 1720 | CB | VAL | A | 264 | −12.428 | −4.470 | 13.950 | 1.00 | 61.31 | A |
| ATOM | 1721 | CG1 | VAL | A | 264 | −11.036 | −4.158 | 14.456 | 1.00 | 60.69 | A |
| ATOM | 1722 | CG2 | VAL | A | 264 | −12.991 | −3.298 | 13.188 | 1.00 | 61.53 | A |
| ATOM | 1723 | C | VAL | A | 264 | −11.713 | −6.845 | 13.791 | 1.00 | 62.89 | A |
| ATOM | 1724 | O | VAL | A | 264 | −12.255 | −7.366 | 14.770 | 1.00 | 61.90 | A |
| ATOM | 1725 | N | LEU | A | 265 | −10.516 | −7.218 | 13.337 | 1.00 | 62.50 | A |
| ATOM | 1726 | CA | LEU | A | 265 | −9.778 | −8.325 | 13.941 | 1.00 | 62.84 | A |
| ATOM | 1727 | CB | LEU | A | 265 | −9.248 | −9.252 | 12.848 | 1.00 | 61.45 | A |
| ATOM | 1728 | CG | LEU | A | 265 | −10.306 | −9.702 | 11.845 | 1.00 | 63.37 | A |
| ATOM | 1729 | CD1 | LEU | A | 265 | −9.653 | −10.413 | 10.680 | 1.00 | 62.64 | A |
| ATOM | 1730 | CD2 | LEU | A | 265 | −11.327 | −10.583 | 12.540 | 1.00 | 63.12 | A |
| ATOM | 1731 | C | LEU | A | 265 | −8.619 | −7.872 | 14.802 | 1.00 | 63.60 | A |
| ATOM | 1732 | O | LEU | A | 265 | −8.363 | −8.425 | 15.865 | 1.00 | 60.89 | A |
| ATOM | 1733 | N | ALA | A | 266 | −7.908 | −6.863 | 14.327 | 1.00 | 67.03 | A |
| ATOM | 1734 | CA | ALA | A | 266 | −6.756 | −6.358 | 15.052 | 1.00 | 69.95 | A |
| ATOM | 1735 | CB | ALA | A | 266 | −5.475 | −6.986 | 14.485 | 1.00 | 69.42 | A |
| ATOM | 1736 | C | ALA | A | 266 | −6.652 | −4.833 | 15.021 | 1.00 | 71.81 | A |
| ATOM | 1737 | O | ALA | A | 266 | −7.204 | −4.166 | 14.138 | 1.00 | 72.60 | A |
| ATOM | 1738 | N | LEU | A | 267 | −5.948 | −4.300 | 16.014 | 1.00 | 73.05 | A |
| ATOM | 1739 | CA | LEU | A | 267 | −5.706 | −2.872 | 16.150 | 1.00 | 74.82 | A |
| ATOM | 1740 | CB | LEU | A | 267 | −6.443 | −2.305 | 17.360 | 1.00 | 74.39 | A |
| ATOM | 1741 | CG | LEU | A | 267 | −7.954 | −2.135 | 17.276 | 1.00 | 73.36 | A |
| ATOM | 1742 | CD1 | LEU | A | 267 | −8.466 | −1.640 | 18.611 | 1.00 | 73.25 | A |
| ATOM | 1743 | CD2 | LEU | A | 267 | −8.300 | −1.149 | 16.173 | 1.00 | 73.15 | A |
| ATOM | 1744 | C | LEU | A | 267 | −4.208 | −2.706 | 16.353 | 1.00 | 76.79 | A |
| ATOM | 1745 | O | LEU | A | 267 | −3.554 | −3.574 | 16.933 | 1.00 | 77.89 | A |
| ATOM | 1746 | N | SER | A | 268 | −3.661 | −1.597 | 15.877 | 1.00 | 78.64 | A |
| ATOM | 1747 | CA | SER | A | 268 | −2.231 | −1.354 | 16.007 | 1.00 | 80.04 | A |
| ATOM | 1748 | CB | SER | A | 268 | −1.474 | −1.960 | 14.827 | 1.00 | 79.09 | A |
| ATOM | 1749 | OG | SER | A | 268 | −1.997 | −3.227 | 14.479 | 1.00 | 81.23 | A |
| ATOM | 1750 | C | SER | A | 268 | −1.975 | .132 | 16.017 | 1.00 | 81.61 | A |
| ATOM | 1751 | O | SER | A | 268 | −2.355 | .843 | 15.083 | 1.00 | 82.86 | A |
| ATOM | 1752 | N | CYS | A | 269 | −1.340 | .617 | 17.070 | 1.00 | 82.68 | A |
| ATOM | 1753 | CA | CYS | A | 269 | −1.031 | 2.029 | 17.120 | 1.00 | 85.83 | A |
| ATOM | 1754 | CB | CYS | A | 269 | −1.704 | 2.690 | 18.319 | 1.00 | 85.06 | A |
| ATOM | 1755 | SG | CYS | A | 269 | −1.180 | 2.040 | 19.882 | 1.00 | 83.60 | A |
| ATOM | 1756 | C | CYS | A | 269 | .482 | 2.242 | 17.169 | 1.00 | 88.36 | A |
| ATOM | 1757 | O | CYS | A | 269 | 1.184 | 1.683 | 18.016 | 1.00 | 88.80 | A |
| ATOM | 1758 | N | SER | A | 270 | .972 | 3.042 | 16.228 | 1.00 | 90.78 | A |
| ATOM | 1759 | CA | SER | A | 270 | 2.385 | 3.370 | 16.121 | 1.00 | 92.20 | A |
| ATOM | 1760 | CB | SER | A | 270 | 2.827 | 3.266 | 14.664 | 1.00 | 92.28 | A |
| ATOM | 1761 | OG | SER | A | 270 | 2.061 | 4.129 | 13.841 | 1.00 | 92.76 | A |
| ATOM | 1762 | C | SER | A | 270 | 2.596 | 4.794 | 16.631 | 1.00 | 93.38 | A |
| ATOM | 1763 | O | SER | A | 270 | 1.804 | 5.689 | 16.344 | 1.00 | 93.84 | A |
| ATOM | 1764 | N | CYS | A | 271 | 3.663 | 4.997 | 17.394 | 1.00 | 94.99 | A |
| ATOM | 1765 | CA | CYS | A | 271 | 3.959 | 6.309 | 17.948 | 1.00 | 95.99 | A |
| ATOM | 1766 | CB | CYS | A | 271 | 4.125 | 6.213 | 19.474 | 1.00 | 96.90 | A |
| ATOM | 1767 | SG | CYS | A | 271 | 2.696 | 5.529 | 20.390 | 1.00 | 98.52 | A |
| ATOM | 1768 | C | CYS | A | 271 | 5.232 | 6.860 | 17.317 | 1.00 | 96.27 | A |
| ATOM | 1769 | O | CYS | A | 271 | 5.489 | 6.646 | 16.127 | 1.00 | 95.07 | A |
| ATOM | 1770 | N | ALA | A | 272 | 6.015 | 7.570 | 18.129 | 1.00 | 97.28 | A |
| ATOM | 1771 | CA | ALA | A | 272 | 7.279 | 8.175 | 17.710 | 1.00 | 98.26 | A |
| ATOM | 1772 | CB | ALA | A | 272 | 8.431 | 7.205 | 17.976 | 1.00 | 98.81 | A |
| ATOM | 1773 | C | ALA | A | 272 | 7.295 | 8.616 | 16.247 | .00 | 98.47 | A |
| ATOM | 1774 | O | ALA | A | 272 | 6.266 | 9.003 | 15.692 | 1.00 | 98.80 | A |
| ATOM | 1775 | N | ALA | A | 273 | 8.474 | 8.549 | 15.633 | .00 | 98.77 | A |
| ATOM | 1776 | CA | ALA | A | 273 | 8.664 | 8.943 | 14.239 | 1.00 | 98.99 | A |
| ATOM | 1777 | CB | ALA | A | 273 | 7.612 | 8.274 | 13.346 | 1.00 | 99.01 | A |
| ATOM | 1778 | C | ALA | A | 273 | 8.595 | 10.465 | 14.092 | 1.00 | 99.45 | A |
| ATOM | 1779 | O | ALA | A | 273 | 8.939 | 11.202 | 15.024 | 1.00 | 99.71 | A |
| ATOM | 1780 | N | ALA | A | 274 | 8.153 | 10.924 | 12.919 | 1.00 | 99.52 | A |
| ATOM | 1781 | CA | ALA | A | 274 | 8.034 | 12.356 | 12.621 | 1.00 | 98.52 | A |
| ATOM | 1782 | CB | ALA | A | 274 | 8.843 | 12.701 | 11.361 | 1.00 | 97.99 | A |
| ATOM | 1783 | C | ALA | A | 274 | 6.574 | 12.784 | 12.441 | 1.00 | 97.73 | A |
| ATOM | 1784 | O | ALA | A | 274 | 5.998 | 12.642 | 11.360 | 1.00 | 97.69 | A |
| ATOM | 1785 | N | ALA | A | 275 | 5.984 | 13.305 | 13.510 | .00 | 96.95 | A |
| ATOM | 1786 | CA | ALA | A | 275 | 4.599 | 13.757 | 13.478 | .00 | 96.10 | A |
| ATOM | 1787 | CB | ALA | A | 275 | 3.690 | 12.633 | 12.983 | .00 | 96.11 | A |
| ATOM | 1788 | C | ALA | A | 275 | 4.157 | 14.218 | 14.863 | .00 | 95.52 | A |
| ATOM | 1789 | O | ALA | A | 275 | 4.671 | 15.201 | 15.395 | .00 | 95.46 | A |
| ATOM | 1790 | N | ALA | A | 276 | 3.202 | 13.497 | 15.440 | 1.00 | 94.67 | A |
| ATOM | 1791 | CA | ALA | A | 276 | 2.672 | 13.812 | 16.764 | 1.00 | 94.17 | A |
| ATOM | 1792 | CB | ALA | A | 276 | 1.991 | 15.170 | 16.745 | 1.00 | 94.42 | A |
| ATOM | 1793 | C | ALA | A | 276 | 1.683 | 12.725 | 17.191 | 1.00 | 93.76 | A |
| ATOM | 1794 | O | ALA | A | 276 | 2.053 | 11.553 | 17.250 | 1.00 | 95.59 | A |
| ATOM | 1795 | N | ALA | A | 277 | .439 | 13.108 | 17.490 | 1.00 | 91.55 | A |
| ATOM | 1796 | CA | ALA | A | 277 | −.598 | 12.152 | 17.897 | 1.00 | 88.74 | A |
| ATOM | 1797 | CB | ALA | A | 277 | −1.983 | 12.700 | 17.535 | 1.00 | 89.02 | A |
| ATOM | 1798 | C | ALA | A | 277 | −.376 | 10.798 | 17.214 | 1.00 | 86.78 | A |
| ATOM | 1799 | O | ALA | A | 277 | −.240 | 10.734 | 15.989 | 1.00 | 87.51 | A |
| ATOM | 1800 | N | ALA | A | 278 | −.339 | 9.724 | 18.004 | 1.00 | 83.58 | A |
| ATOM | 1801 | CA | ALA | A | 278 | −.111 | 8.371 | 17.481 | 1.00 | 79.61 | A |
| ATOM | 1802 | CB | ALA | A | 278 | −.291 | 7.331 | 18.605 | 1.00 | 79.90 | A |
| ATOM | 1803 | C | ALA | A | 278 | −1.005 | 8.024 | 16.290 | 1.00 | 76.42 | A |
| ATOM | 1804 | O | ALA | A | 278 | −1.965 | 8.728 | 15.992 | 1.00 | 75.78 | A |
| ATOM | 1805 | N | SER | A | 279 | −.688 | 6.931 | 15.607 | 1.00 | 73.01 | A |
| ATOM | 1806 | CA | SER | A | 279 | −1.459 | 6.520 | 14.444 | 1.00 | 70.17 | A |
| ATOM | 1807 | CB | SER | A | 279 | −.564 | 6.569 | 13.204 | 1.00 | 69.56 | A |
| ATOM | 1808 | OG | SER | A | 279 | −1.316 | 6.382 | 12.016 | 1.00 | 69.76 | A |
| ATOM | 1809 | C | SER | A | 279 | −2.029 | 5.115 | 14.620 | 1.00 | 68.35 | A |
| ATOM | 1810 | O | SER | A | 279 | −1.304 | 4.189 | 14.956 | 1.00 | 68.24 | A |
| ATOM | 1811 | N | TRP | A | 280 | −3.326 | 4.946 | 14.392 | 1.00 | 65.70 | A |
| ATOM | 1812 | CA | TRP | A | 280 | −3.928 | 3.631 | 14.551 | 1.00 | 64.16 | A |
| ATOM | 1813 | CB | TRP | A | 280 | −5.217 | 3.721 | 15.358 | 1.00 | 59.66 | A |
| ATOM | 1814 | CG | TRP | A | 280 | −5.055 | 4.089 | 16.799 | 1.00 | 55.21 | A |
| ATOM | 1815 | CD2 | TRP | A | 280 | −5.030 | 3.187 | 17.907 | 1.00 | 52.55 | A |
| ATOM | 1816 | CE2 | TRP | A | 280 | −4.961 | 3.972 | 19.083 | 1.00 | 52.33 | A |
| ATOM | 1817 | CE3 | TRP | A | 280 | −5.064 | 1.792 | 18.022 | 1.00 | 50.93 | A |
| ATOM | 1818 | CD1 | TRP | A | 280 | −4.988 | 5.350 | 17.329 | 1.00 | 53.17 | A |
| ATOM | 1819 | NE1 | TRP | A | 280 | −4.939 | 5.287 | 18.699 | 1.00 | 51.36 | A |
| ATOM | 1820 | CZ2 | TRP | A | 280 | −4.925 | 3.402 | 20.368 | 1.00 | 52.04 | A |
| ATOM | 1821 | CZ3 | TRP | A | 280 | −5.029 | 1.225 | 19.300 | 1.00 | 52.57 | A |
| ATOM | 1822 | CH2 | TRP | A | 280 | −4.960 | 2.033 | 20.455 | 1.00 | 51.36 | A |
| ATOM | 1823 | C | TRP | A | 280 | −4.254 | 3.006 | 13.214 | 1.00 | 66.89 | A |
| ATOM | 1824 | O | TRP | A | 280 | −4.602 | 3.714 | 12.270 | 1.00 | 67.78 | A |
| ATOM | 1825 | N | SER | A | 281 | −4.152 | 1.679 | 13.136 | 1.00 | 69.69 | A |
| ATOM | 1826 | CA | SER | A | 281 | −4.469 | .948 | 11.903 | 1.00 | 72.25 | A |
| ATOM | 1827 | CB | SER | A | 281 | −3.186 | .444 | 11.222 | 1.00 | 73.41 | A |
| ATOM | 1828 | OG | SER | A | 281 | −2.482 | −.472 | 12.042 | 1.00 | 75.64 | A |
| ATOM | 1829 | C | SER | A | 281 | −5.410 | −.221 | 12.208 | 1.00 | 72.28 | A |
| ATOM | 1830 | O | SER | A | 281 | −5.263 | −.910 | 13.209 | 1.00 | 71.66 | A |
| ATOM | 1831 | N | ILE | A | 282 | −6.376 | −.447 | 11.335 | 1.00 | 73.83 | A |
| ATOM | 1832 | CA | ILE | A | 282 | −7.343 | −1.506 | 11.566 | 1.00 | 75.65 | A |
| ATOM | 1833 | CB | ILE | A | 282 | −8.773 | −.932 | 11.569 | 1.00 | 76.14 | A |
| ATOM | 1834 | CG2 | ILE | A | 282 | −9.795 | −2.043 | 11.700 | 1.00 | 75.42 | A |
| ATOM | 1835 | CG1 | ILE | A | 282 | −8.909 | .078 | 12.707 | 1.00 | 76.11 | A |
| ATOM | 1836 | CD1 | ILE | A | 282 | −10.246 | .760 | 12.747 | 1.00 | 77.97 | A |
| ATOM | 1837 | C | ILE | A | 282 | −7.275 | −2.624 | 10.550 | 1.00 | 76.68 | A |
| ATOM | 1838 | O | ILE | A | 282 | −7.221 | −2.381 | 9.349 | 1.00 | 76.36 | A |
| ATOM | 1839 | N | ILE | A | 283 | −7.279 | −3.855 | 11.052 | 1.00 | 78.29 | A |
| ATOM | 1840 | CA | ILE | A | 283 | −7.245 | −5.032 | 10.198 | 1.00 | 79.63 | A |
| ATOM | 1841 | CB | ILE | A | 283 | −6.164 | −6.062 | 10.644 | 1.00 | 80.13 | A |
| ATOM | 1842 | CG2 | ILE | A | 283 | −5.967 | −7.096 | 9.550 | 1.00 | 79.51 | A |
| ATOM | 1843 | CG1 | ILE | A | 283 | −4.822 | −5.373 | 10.916 | 1.00 | 80.65 | A |
| ATOM | 1844 | CD1 | ILE | A | 283 | −4.744 | −4.657 | 12.258 | 1.00 | 81.05 | A |
| ATOM | 1845 | C | ILE | A | 283 | −8.613 | −5.694 | 10.302 | 1.00 | 79.90 | A |
| ATOM | 1846 | O | ILE | A | 283 | −8.907 | −6.381 | 11.277 | 1.00 | 79.61 | A |
| ATOM | 1847 | N | SER | A | 284 | −9.459 | −5.462 | 9.309 | 1.00 | 80.92 | A |
| ATOM | 1848 | CA | SER | A | 284 | −10.789 | −6.050 | 9.314 | 1.00 | 82.43 | A |
| ATOM | 1849 | CB | SER | A | 284 | −11.858 | −4.986 | 9.061 | 1.00 | 82.16 | A |
| ATOM | 1850 | OG | SER | A | 284 | −11.703 | −4.415 | 7.774 | 1.00 | 83.19 | A |
| ATOM | 1851 | C | SER | A | 284 | −10.830 | −7.081 | 8.209 | 1.00 | 82.87 | A |
| ATOM | 1852 | O | SER | A | 284 | −10.155 | −6.927 | 7.194 | 1.00 | 83.40 | A |
| ATOM | 1853 | N | ALA | A | 285 | −11.619 | −8.130 | 8.410 | 1.00 | 83.41 | A |
| ATOM | 1854 | CA | ALA | A | 285 | −11.735 | −9.193 | 7.425 | 1.00 | 84.29 | A |
| ATOM | 1855 | CB | ALA | A | 285 | −12.092 | −10.497 | 8.108 | 1.00 | 83.81 | A |
| ATOM | 1856 | C | ALA | A | 285 | −12.774 | −8.863 | 6.367 | 1.00 | 85.02 | A |
| ATOM | 1857 | O | ALA | A | 285 | −13.372 | −7.782 | 6.368 | 1.00 | 85.19 | A |
| ATOM | 1858 | N | SER | A | 286 | −12.980 | −9.814 | 5.461 | .50 | 85.06 | A |
| ATOM | 1859 | CA | SER | A | 286 | −13.942 | −9.657 | 4.380 | .50 | 84.01 | A |
| ATOM | 1860 | CB | SER | A | 286 | −15.357 | −9.595 | 4.948 | .50 | 83.93 | A |
| ATOM | 1861 | OG | SER | A | 286 | −15.611 | −10.727 | 5.762 | .50 | 84.30 | A |
| ATOM | 1862 | C | SER | A | 286 | −13.646 | −8.399 | 3.579 | .50 | 83.35 | A |
| ATOM | 1863 | O | SER | A | 286 | −14.547 | −7.621 | 3.270 | .50 | 83.08 | A |
| ATOM | 1864 | N | ALA | A | 287 | −12.371 | −8.197 | 3.263 | .50 | 82.79 | A |
| ATOM | 1865 | CA | ALA | A | 287 | −11.956 | −7.046 | 2.475 | .50 | 82.85 | A |
| ATOM | 1866 | CB | ALA | A | 287 | −10.445 | −7.042 | 2.317 | .50 | 82.67 | A |
| ATOM | 1867 | C | ALA | A | 287 | −12.635 | −7.220 | 1.123 | .50 | 83.14 | A |
| ATOM | 1868 | O | ALA | A | 287 | −12.793 | −6.274 | .347 | .50 | 82.95 | A |
| ATOM | 1869 | N | ALA | A | 288 | −13.028 | −8.464 | .867 | .50 | 83.32 | A |
| ATOM | 1870 | CA | ALA | A | 288 | −13.715 | −8.871 | −.351 | .50 | 83.34 | A |
| ATOM | 1871 | CB | ALA | A | 288 | −12.705 | −9.178 | −1.456 | .50 | 82.85 | A |
| ATOM | 1872 | C | ALA | A | 288 | −14.461 | −10.137 | .058 | .50 | 83.64 | A |
| ATOM | 1873 | O | ALA | A | 288 | −15.515 | −10.071 | .694 | .50 | 83.49 | A |
| ATOM | 1874 | N | ALA | A | 289 | −13.899 | −11.286 | −.303 | .50 | 84.00 | A |
| ATOM | 1875 | CA | ALA | A | 289 | −14.475 | −12.577 | .047 | .50 | 84.12 | A |
| ATOM | 1876 | CB | ALA | A | 289 | −15.015 | −13.267 | −1.187 | .50 | 83.42 | A |
| ATOM | 1877 | C | ALA | A | 289 | −13.330 | −13.380 | .646 | .50 | 84.87 | A |
| ATOM | 1878 | O | ALA | A | 289 | −12.520 | −13.952 | −.085 | .50 | 84.50 | A |
| ATOM | 1879 | N | ALA | A | 290 | −13.259 | −13.401 | 1.976 | .50 | 85.91 | A |
| ATOM | 1880 | CA | ALA | A | 290 | −12.198 | −14.110 | 2.688 | .50 | 87.01 | A |
| ATOM | 1881 | CB | ALA | A | 290 | −12.128 | −15.565 | 2.227 | .50 | 86.85 | A |
| ATOM | 1882 | C | ALA | A | 290 | −10.869 | −13.400 | 2.424 | .50 | 87.96 | A |
| ATOM | 1883 | O | ALA | A | 290 | −9.856 | −14.027 | 2.097 | .50 | 87.60 | A |
| ATOM | 1884 | N | ALA | A | 291 | −10.891 | −12.078 | 2.564 | .50 | 88.93 | A |
| ATOM | 1885 | CA | ALA | A | 291 | −9.708 | −11.256 | 2.348 | .50 | 89.87 | A |
| ATOM | 1886 | CB | ALA | A | 291 | −9.898 | −10.387 | 1.103 | .50 | 88.63 | A |
| ATOM | 1887 | C | ALA | A | 291 | −9.452 | −10.380 | 3.579 | .50 | 90.86 | A |
| ATOM | 1888 | O | ALA | A | 291 | −10.329 | −10.221 | 4.437 | .50 | 90.55 | A |
| ATOM | 1889 | N | ALA | A | 292 | −8.246 | −9.824 | 3.666 | .50 | 91.85 | A |
| ATOM | 1890 | CA | ALA | A | 292 | −7.875 | −8.967 | 4.786 | .00 | 93.01 | A |
| ATOM | 1891 | CB | ALA | A | 292 | −6.922 | −9.707 | 5.716 | .00 | 92.91 | A |
| ATOM | 1892 | C | ALA | A | 292 | −7.222 | −7.687 | 4.277 | .00 | 93.95 | A |
| ATOM | 1893 | O | ALA | A | 292 | −6.636 | −7.670 | 3.194 | .00 | 93.98 | A |
| ATOM | 1894 | N | GLU | A | 293 | −7.323 | −6.616 | 5.059 | 1.00 | 95.01 | A |
| ATOM | 1895 | CA | GLU | A | 293 | −6.736 | −5.335 | 4.666 | 1.00 | 96.79 | A |
| ATOM | 1896 | CB | GLU | A | 293 | −7.629 | −4.628 | 3.636 | 1.00 | 98.07 | A |
| ATOM | 1897 | CG | GLU | A | 293 | −9.057 | −4.357 | 4.094 | 1.00 | 98.84 | A |
| ATOM | 1898 | CD | GLU | A | 293 | −9.920 | −3.810 | 2.972 | 1.00 | 99.31 | A |
| ATOM | 1899 | OE1 | GLU | A | 293 | −9.474 | −2.857 | 2.297 | 1.00 | 100.00 | A |
| ATOM | 1900 | OE2 | GLU | A | 293 | −11.041 | −4.325 | 2.767 | 1.00 | 98.68 | A |
| ATOM | 1901 | C | GLU | A | 293 | −6.476 | −4.410 | 5.850 | 1.00 | 96.59 | A |
| ATOM | 1902 | O | GLU | A | 293 | −6.616 | −4.813 | 7.006 | 1.00 | 97.21 | A |
| ATOM | 1903 | N | GLU | A | 294 | −6.109 | −3.164 | 5.563 | 1.00 | 96.24 | A |
| ATOM | 1904 | CA | GLU | A | 294 | −5.805 | −2.211 | 6.627 | 1.00 | 95.24 | A |
| ATOM | 1905 | CB | GLU | A | 294 | −4.338 | −2.377 | 7.027 | 1.00 | 95.80 | A |
| ATOM | 1906 | CG | GLU | A | 294 | −3.900 | −1.520 | 8.184 | 1.00 | 97.46 | A |
| ATOM | 1907 | CD | GLU | A | 294 | −2.487 | −1.824 | 8.615 | 1.00 | 98.30 | A |
| ATOM | 1908 | OE1 | GLU | A | 294 | −1.556 | −1.599 | 7.808 | 1.00 | 98.82 | A |
| ATOM | 1909 | OE2 | GLU | A | 294 | −2.313 | −2.296 | 9.761 | 1.00 | 98.28 | A |
| ATOM | 1910 | C | GLU | A | 294 | −6.085 | −.750 | 6.267 | 1.00 | 93.57 | A |
| ATOM | 1911 | O | GLU | A | 294 | −6.395 | −.431 | 5.120 | 1.00 | 93.93 | A |
| ATOM | 1912 | N | GLU | A | 295 | −5.981 | .122 | 7.268 | 1.00 | 91.64 | A |
| ATOM | 1913 | CA | GLU | A | 295 | −6.192 | 1.560 | 7.118 | 1.00 | 89.96 | A |
| ATOM | 1914 | CB | GLU | A | 295 | −7.680 | 1.872 | 6.954 | 1.00 | 91.57 | A |
| ATOM | 1915 | CG | GLU | A | 295 | −8.293 | 1.416 | 5.640 | 1.00 | 92.37 | A |
| ATOM | 1916 | CD | GLU | A | 295 | −9.735 | 1.848 | 5.502 | 1.00 | 93.38 | A |
| ATOM | 1917 | OE1 | GLU | A | 295 | −9.994 | 3.072 | 5.533 | 1.00 | 93.94 | A |
| ATOM | 1918 | OE2 | GLU | A | 295 | −10.609 | .966 | 5.365 | 1.00 | 94.09 | A |
| ATOM | 1919 | C | GLU | A | 295 | −5.662 | 2.270 | 8.365 | 1.00 | 88.24 | A |
| ATOM | 1920 | O | GLU | A | 295 | −5.702 | 1.707 | 9.461 | 1.00 | 88.46 | A |
| ATOM | 1921 | N | ALA | A | 296 | −5.174 | 3.500 | 8.202 | 1.00 | 85.10 | A |
| ATOM | 1922 | CA | ALA | A | 296 | −4.635 | 4.268 | 9.330 | 1.00 | 81.51 | A |
| ATOM | 1923 | CB | ALA | A | 296 | −3.263 | 4.828 | 8.961 | 1.00 | 81.36 | A |
| ATOM | 1924 | C | ALA | A | 296 | −5.576 | 5.405 | 9.754 | 1.00 | 79.07 | A |
| ATOM | 1925 | O | ALA | A | 296 | −6.128 | 6.093 | 8.894 | 1.00 | 79.42 | A |
| ATOM | 1926 | N | PHE | A | 297 | −5.753 | 5.592 | 11.069 | 1.00 | 75.17 | A |
| ATOM | 1927 | CA | PHE | A | 297 | −6.632 | 6.638 | 11.622 | 1.00 | 71.06 | A |
| ATOM | 1928 | CB | PHE | A | 297 | −7.936 | 6.031 | 12.169 | 1.00 | 68.77 | A |
| ATOM | 1929 | CG | PHE | A | 297 | −8.713 | 5.231 | 11.164 | 1.00 | 65.58 | A |
| ATOM | 1930 | CD1 | PHE | A | 297 | −8.354 | 3.928 | 10.859 | 1.00 | 65.37 | A |
| ATOM | 1931 | CD2 | PHE | A | 297 | −9.794 | 5.789 | 10.502 | 1.00 | 65.41 | A |
| ATOM | 1932 | CE1 | PHE | A | 297 | −9.059 | 3.197 | 9.906 | 1.00 | 63.71 | A |
| ATOM | 1933 | CE2 | PHE | A | 297 | −10.504 | 5.061 | 9.549 | 1.00 | 63.67 | A |
| ATOM | 1934 | CZ | PHE | A | 297 | −10.135 | 3.771 | 9.252 | 1.00 | 62.91 | A |
| ATOM | 1935 | C | PHE | A | 297 | −5.939 | 7.394 | 12.755 | 1.00 | 69.60 | A |
| ATOM | 1936 | O | PHE | A | 297 | −4.844 | 7.022 | 13.163 | 1.00 | 69.79 | A |
| ATOM | 1937 | N | ASP | A | 298 | −6.590 | 8.439 | 13.269 | 1.00 | 67.82 | A |
| ATOM | 1938 | CA | ASP | A | 298 | −6.044 | 9.254 | 14.361 | 1.00 | 65.69 | A |
| ATOM | 1939 | CB | ASP | A | 298 | −6.281 | 10.744 | 14.115 | 1.00 | 68.55 | A |
| ATOM | 1940 | CG | ASP | A | 298 | −5.603 | 11.242 | 12.881 | 1.00 | 70.59 | A |
| ATOM | 1941 | OD1 | ASP | A | 298 | −4.413 | 10.909 | 12.691 | 1.00 | 73.29 | A |
| ATOM | 1942 | OD2 | ASP | A | 298 | −6.260 | 11.975 | 12.114 | 1.00 | 71.72 | A |
| ATOM | 1943 | C | ASP | A | 298 | −6.658 | 8.936 | 15.711 | 1.00 | 62.93 | A |
| ATOM | 1944 | O | ASP | A | 298 | −6.065 | 9.188 | 16.751 | 1.00 | 62.97 | A |
| ATOM | 1945 | N | ALA | A | 299 | −7.871 | 8.421 | 15.693 | 1.00 | 60.72 | A |
| ATOM | 1946 | CA | ALA | A | 299 | −8.562 | 8.089 | 16.922 | 1.00 | 59.18 | A |
| ATOM | 1947 | CB | ALA | A | 299 | −9.385 | 9.275 | 17.403 | 1.00 | 58.72 | A |
| ATOM | 1948 | C | ALA | A | 299 | −9.458 | 6.915 | 16.599 | 1.00 | 58.36 | A |
| ATOM | 1949 | O | ALA | A | 299 | −9.764 | 6.664 | 15.429 | 1.00 | 58.50 | A |
| ATOM | 1950 | N | VAL | A | 300 | −9.878 | 6.196 | 17.631 | 1.00 | 56.41 | A |
| ATOM | 1951 | CA | VAL | A | 300 | −10.721 | 5.031 | 17.443 | 1.00 | 53.99 | A |
| ATOM | 1952 | CB | VAL | A | 300 | −9.843 | 3.747 | 17.444 | 1.00 | 54.35 | A |
| ATOM | 1953 | CG1 | VAL | A | 300 | −10.700 | 2.494 | 17.474 | 1.00 | 55.84 | A |
| ATOM | 1954 | CG2 | VAL | A | 300 | −8.974 | 3.728 | 16.215 | 1.00 | 54.39 | A |
| ATOM | 1955 | C | VAL | A | 300 | −11.774 | 4.939 | 18.535 | 1.00 | 52.27 | A |
| ATOM | 1956 | O | VAL | A | 300 | −11.445 | 4.941 | 19.715 | 1.00 | 50.38 | A |
| ATOM | 1957 | N | ILE | A | 301 | −13.043 | 4.888 | 18.151 | 1.00 | 52.40 | A |
| ATOM | 1958 | CA | ILE | A | 301 | −14.102 | 4.749 | 19.142 | 1.00 | 54.34 | A |
| ATOM | 1959 | CB | ILE | A | 301 | −15.300 | 5.643 | 18.842 | 1.00 | 52.94 | A |
| ATOM | 1960 | CG2 | ILE | A | 301 | −16.430 | 5.331 | 19.807 | 1.00 | 52.03 | A |
| ATOM | 1961 | CG1 | ILE | A | 301 | −14.883 | 7.106 | 18.988 | 1.00 | 52.26 | A |
| ATOM | 1962 | CD1 | ILE | A | 301 | −15.966 | 8.087 | 18.687 | 1.00 | 51.92 | A |
| ATOM | 1963 | C | ILE | A | 301 | −14.553 | 3.300 | 19.160 | 1.00 | 57.58 | A |
| ATOM | 1964 | O | ILE | A | 301 | −15.030 | 2.772 | 18.156 | 1.00 | 59.47 | A |
| ATOM | 1965 | N | MSE | A | 302 | −14.368 | 2.661 | 20.310 | 1.00 | 60.03 | A |
| ATOM | 1966 | CA | MSE | A | 302 | −14.714 | 1.263 | 20.526 | 1.00 | 62.14 | A |
| ATOM | 1967 | CB | MSE | A | 302 | −13.789 | .700 | 21.605 | 1.00 | 67.97 | A |
| ATOM | 1968 | CG | MSE | A | 302 | −13.913 | −.781 | 21.844 | 1.00 | 74.62 | A |
| ATOM | 1969 | SE | MSE | A | 302 | −13.239 | −1.752 | 20.345 | 1.00 | 85.18 | A |
| ATOM | 1970 | CE | MSE | A | 302 | −11.364 | −1.769 | 20.863 | 1.00 | 78.31 | A |
| ATOM | 1971 | C | MSE | A | 302 | −16.174 | 1.138 | 20.970 | 1.00 | 61.43 | A |
| ATOM | 1972 | O | MSE | A | 302 | −16.568 | 1.656 | 22.013 | 1.00 | 61.86 | A |
| ATOM | 1973 | N | THR | A | 303 | −16.973 | .424 | 20.197 | 1.00 | 59.80 | A |
| ATOM | 1974 | CA | THR | A | 303 | −18.373 | .291 | 20.527 | 1.00 | 59.36 | A |
| ATOM | 1975 | CB | THR | A | 303 | −19.197 | 1.112 | 19.506 | 1.00 | 60.60 | A |
| ATOM | 1976 | OG1 | THR | A | 303 | −19.252 | 2.477 | 19.956 | 1.00 | 58.61 | A |
| ATOM | 1977 | CG2 | THR | A | 303 | −20.617 | .541 | 19.295 | 1.00 | 60.40 | A |
| ATOM | 1978 | C | THR | A | 303 | −18.876 | −1.145 | 20.641 | 1.00 | 60.03 | A |
| ATOM | 1979 | O | THR | A | 303 | −19.999 | −1.379 | 21.078 | 1.00 | 60.73 | A |
| ATOM | 1980 | N | ALA | A | 304 | −18.047 | −2.115 | 20.280 | 1.00 | 60.14 | A |
| ATOM | 1981 | CA | ALA | A | 304 | −18.474 | −3.503 | 20.368 | 1.00 | 60.27 | A |
| ATOM | 1982 | CB | ALA | A | 304 | −17.531 | −4.406 | 19.572 | 1.00 | 60.13 | A |
| ATOM | 1983 | C | ALA | A | 304 | −18.501 | −3.927 | 21.827 | 1.00 | 60.55 | A |
| ATOM | 1984 | O | ALA | A | 304 | −17.917 | −3.267 | 22.684 | 1.00 | 59.51 | A |
| ATOM | 1985 | N | PRO | A | 305 | −19.201 | −5.028 | 22.133 | 1.00 | 61.32 | A |
| ATOM | 1986 | CD | PRO | A | 305 | −20.196 | −5.717 | 21.296 | 1.00 | 62.73 | A |
| ATOM | 1987 | CA | PRO | A | 305 | −19.278 | −5.512 | 23.510 | 1.00 | 61.18 | A |
| ATOM | 1986 | CB | PRO | A | 305 | −20.188 | −6.724 | 23.393 | 1.00 | 60.95 | A |
| ATOM | 1989 | CG | PRO | A | 305 | −21.149 | −6.279 | 22.349 | 1.00 | 63.23 | A |
| ATOM | 1990 | C | PRO | A | 305 | −17.908 | −5.864 | 24.056 | 1.00 | 61.29 | A |
| ATOM | 1991 | O | PRO | A | 305 | −17.008 | −6.249 | 23.313 | 1.00 | 60.81 | A |
| ATOM | 1992 | N | LEU | A | 306 | −17.761 | −5.731 | 25.367 | 1.00 | 62.04 | A |
| ATOM | 1993 | CA | LEU | A | 306 | −16.505 | −6.014 | 26.017 | 1.00 | 62.68 | A |
| ATOM | 1994 | CB | LEU | A | 306 | −16.623 | −5.723 | 27.508 | 1.00 | 60.88 | A |
| ATOM | 1995 | CG | LEU | A | 306 | −15.401 | −5.023 | 28.096 | 1.00 | 59.89 | A |
| ATOM | 1996 | CD1 | LEU | A | 306 | −14.818 | −4.046 | 27.102 | 1.00 | 58.58 | A |
| ATOM | 1997 | CD2 | LEU | A | 306 | −15.806 | −4.307 | 29.364 | 1.00 | 60.19 | A |
| ATOM | 1998 | C | LEU | A | 306 | −16.046 | −7.443 | 25.769 | 1.00 | 63.98 | A |
| ATOM | 1999 | O | LEU | A | 306 | −14.848 | −7.713 | 25.764 | 1.00 | 65.24 | A |
| ATOM | 2000 | N | CYS | A | 307 | −16.980 | −8.361 | 25.544 | 1.00 | 64.24 | A |
| ATOM | 2001 | CA | CYS | A | 307 | −16.587 | −9.738 | 25.275 | 1.00 | 64.89 | A |
| ATOM | 2002 | CB | CYS | A | 307 | −17.775 | −10.683 | 25.425 | 1.00 | 64.10 | A |
| ATOM | 2003 | SG | CYS | A | 307 | −18.880 | −10.736 | 24.000 | 1.00 | 63.26 | A |
| ATOM | 2004 | C | CYS | A | 307 | −16.013 | −9.869 | 23.861 | 1.00 | 66.36 | A |
| ATOM | 2005 | O | CYS | A | 307 | −15.407 | −10.880 | 23.540 | 1.00 | 66.81 | A |
| ATOM | 2006 | N | ASP | A | 308 | −16.212 | −8.850 | 23.021 | 1.00 | 68.67 | A |
| ATOM | 2007 | CA | ASP | A | 308 | −15.701 | −8.854 | 21.644 | 1.00 | 69.93 | A |
| ATOM | 2008 | CB | ASP | A | 308 | −16.687 | −8.208 | 20.672 | 1.00 | 69.96 | A |
| ATOM | 2009 | CG | ASP | A | 308 | −17.720 | −9.172 | 20.173 | 1.00 | 70.09 | A |
| ATOM | 2010 | OD1 | ASP | A | 308 | −17.447 | −10.391 | 20.173 | 1.00 | 70.91 | A |
| ATOM | 2011 | OD2 | ASP | A | 308 | −18.800 | −8.708 | 19.766 | 1.00 | 70.98 | A |
| ATOM | 2012 | C | ASP | A | 308 | −14.385 | −8.111 | 21.536 | 1.00 | 71.20 | A |
| ATOM | 2013 | O | ASP | A | 308 | −13.632 | −8.294 | 20.578 | 1.00 | 70.81 | A |
| ATOM | 2014 | N | VAL | A | 309 | −14.125 | −7.250 | 22.508 | 1.00 | 72.19 | A |
| ATOM | 2015 | CA | VAL | A | 309 | −12.889 | −6.501 | 22.524 | 1.00 | 73.74 | A |
| ATOM | 2016 | CB | VAL | A | 309 | −12.940 | −5.381 | 23.575 | 1.00 | 73.80 | A |
| ATOM | 2017 | CG1 | VAL | A | 309 | −11.642 | −4.590 | 23.569 | 1.00 | 74.03 | A |
| ATOM | 2018 | CG2 | VAL | A | 309 | −14.120 | −4.473 | 23.294 | 1.00 | 74.25 | A |
| ATOM | 2019 | C | VAL | A | 309 | −11.785 | −7.484 | 22.880 | 1.00 | 75.06 | A |
| ATOM | 2020 | O | VAL | A | 309 | −10.646 | −7.338 | 22.447 | 1.00 | 75.32 | A |
| ATOM | 2021 | N | LYS | A | 310 | −12.140 | −8.502 | 23.656 | 1.00 | 77.60 | A |
| ATOM | 2022 | CA | LYS | A | 310 | −11.184 | −9.514 | 24.084 | 1.00 | 80.16 | A |
| ATOM | 2023 | CB | LYS | A | 310 | −11.847 | −10.484 | 25.068 | 1.00 | 82.84 | A |
| ATOM | 2024 | CG | LYS | A | 310 | −12.592 | −11.671 | 24.424 | 1.00 | 86.00 | A |
| ATOM | 2025 | CD | LYS | A | 310 | −11.635 | −12.817 | 24.008 | 1.00 | 87.90 | A |
| ATOM | 2026 | CE | LYS | A | 310 | −12.366 | −13.958 | 23.291 | 1.00 | 88.05 | A |
| ATOM | 2027 | NZ | LYS | A | 310 | −11.441 | −15.041 | 22.834 | 1.00 | 87.08 | A |
| ATOM | 2028 | C | LYS | A | 310 | −10.628 | −10.294 | 22.900 | 1.00 | 80.89 | A |
| ATOM | 2029 | O | LYS | A | 310 | −9.420 | −10.506 | 22.800 | 1.00 | 81.39 | A |
| ATOM | 2030 | N | SER | A | 311 | −11.517 | −10.730 | 22.011 | 1.00 | 81.26 | A |
| ATOM | 2031 | CA | SER | A | 311 | −11.118 | −11.497 | 20.836 | 1.00 | 81.23 | A |
| ATOM | 2032 | CB | SER | A | 311 | −12.305 | −12.306 | 20.311 | 1.00 | 80.50 | A |
| ATOM | 2033 | OG | SER | A | 311 | −13.444 | −11.485 | 20.121 | 1.00 | 78.76 | A |
| ATOM | 2034 | C | SER | A | 311 | −10.587 | −10.586 | 19.743 | 1.00 | 82.01 | A |
| ATOM | 2035 | O | SER | A | 311 | −10.864 | −10.791 | 18.568 | 1.00 | 83.24 | A |
| ATOM | 2036 | N | MSE | A | 312 | −9.827 | −9.574 | 20.144 | 1.00 | 82.79 | A |
| ATOM | 2037 | CA | MSE | A | 312 | −9.251 | −8.622 | 19.209 | 1.00 | 83.75 | A |
| ATOM | 2038 | CB | MSE | A | 312 | −9.882 | −7.243 | 19.391 | 1.00 | 85.42 | A |
| ATOM | 2039 | CG | MSE | A | 312 | −11.134 | −7.004 | 18.577 | 1.00 | 88.20 | A |
| ATOM | 2040 | SE | MSE | A | 312 | −11.619 | −5.127 | 18.595 | 1.00 | 92.99 | A |
| ATOM | 2041 | CE | MSE | A | 312 | −13.412 | −5.242 | 19.352 | 1.00 | 90.12 | A |
| ATOM | 2042 | C | MSE | A | 312 | −7.754 | −8.513 | 19.427 | 1.00 | 83.41 | A |
| ATOM | 2043 | O | MSE | A | 312 | −7.308 | −8.121 | 20.499 | 1.00 | 83.70 | A |
| ATOM | 2044 | N | LYS | A | 313 | −6.973 | −8.862 | 18.413 | 1.00 | 83.53 | A |
| ATOM | 2045 | CA | LYS | A | 313 | −5.528 | −8.778 | 18.539 | 1.00 | 83.49 | A |
| ATOM | 2046 | CB | LYS | A | 313 | −4.838 | −9.576 | 17.431 | 1.00 | 82.26 | A |
| ATOM | 2047 | CG | LYS | A | 313 | −4.950 | −11.079 | 17.598 | 1.00 | 82.17 | A |
| ATOM | 2048 | CD | LYS | A | 313 | −4.173 | −11.825 | 16.522 | 1.00 | 81.84 | A |
| ATOM | 2049 | CE | LYS | A | 313 | −4.194 | −13.322 | 16.771 | 1.00 | 80.84 | A |
| ATOM | 2050 | NZ | LYS | A | 313 | −3.448 | −14.057 | 15.719 | 1.00 | 81.46 | A |
| ATOM | 2051 | C | LYS | A | 313 | −5.087 | −7.324 | 18.488 | 1.00 | 84.09 | A |
| ATOM | 2052 | O | LYS | A | 313 | −4.944 | −6.740 | 17.417 | 1.00 | 84.61 | A |
| ATOM | 2053 | N | ILE | A | 314 | −4.872 | −6.746 | 19.664 | 1.00 | 84.82 | A |
| ATOM | 2054 | CA | ILE | A | 314 | −4.446 | −5.357 | 19.783 | 1.00 | 85.81 | A |
| ATOM | 2055 | CB | ILE | A | 314 | −5.420 | −4.589 | 20.704 | 1.00 | 84.35 | A |
| ATOM | 2056 | CG2 | ILE | A | 314 | −5.545 | −5.289 | 22.024 | 1.00 | 84.89 | A |
| ATOM | 2057 | CG1 | ILE | A | 314 | −4.948 | −3.164 | 20.920 | 1.00 | 83.74 | A |
| ATOM | 2058 | CD1 | ILE | A | 314 | −5.966 | −2.343 | 21.634 | 1.00 | 83.94 | A |
| ATOM | 2059 | C | ILE | A | 314 | −3.004 | −5.257 | 20.305 | 1.00 | 87.68 | A |
| ATOM | 2060 | O | ILE | A | 314 | −2.606 | −5.988 | 21.219 | 1.00 | 87.76 | A |
| ATOM | 2061 | N | ALA | A | 315 | −2.222 | −4.356 | 19.713 | 1.00 | 89.14 | A |
| ATOM | 2062 | CA | ALA | A | 315 | −.822 | −4.177 | 20.100 | 1.00 | 90.50 | A |
| ATOM | 2063 | CB | ALA | A | 315 | .078 | −4.879 | 19.089 | 1.00 | 90.90 | A |
| ATOM | 2064 | C | ALA | A | 315 | −.402 | −2.712 | 20.235 | 1.00 | 90.94 | A |
| ATOM | 2065 | O | ALA | A | 315 | −.860 | −1.855 | 19.483 | 1.00 | 91.28 | A |
| ATOM | 2066 | N | LYS | A | 316 | .485 | −2.434 | 21.187 | 1.00 | 91.93 | A |
| ATOM | 2067 | CA | LYS | A | 316 | .968 | −1.073 | 21.426 | 1.00 | 92.79 | A |
| ATOM | 2068 | CB | LYS | A | 316 | 1.531 | −.946 | 22.856 | 1.00 | 93.35 | A |
| ATOM | 2069 | CG | LYS | A | 316 | 2.734 | −1.853 | 23.168 | 1.00 | 94.29 | A |
| ATOM | 2070 | CD | LYS | A | 316 | 3.054 | −1.907 | 24.668 | 1.00 | 94.52 | A |
| ATOM | 2071 | CE | LYS | A | 316 | 1.951 | −2.630 | 25.454 | 1.00 | 93.86 | A |
| ATOM | 2072 | NZ | LYS | A | 316 | 2.230 | −2.698 | 26.920 | 1.00 | 93.77 | A |
| ATOM | 2073 | C | LYS | A | 316 | 2.027 | −.675 | 20.398 | 1.00 | 93.08 | A |
| ATOM | 2074 | O | LYS | A | 316 | 1.734 | −.601 | 19.203 | 1.00 | 92.43 | A |
| ATOM | 2075 | N | ARG | A | 317 | 3.251 | −.423 | 20.864 | 1.00 | 93.49 | A |
| ATOM | 2076 | CA | ARG | A | 317 | 4.355 | −.033 | 19.988 | 1.00 | 93.43 | A |
| ATOM | 2077 | CB | ARG | A | 317 | 5.635 | .153 | 20.808 | 1.00 | 93.39 | A |
| ATOM | 2078 | CG | ARG | A | 317 | 6.859 | .574 | 20.002 | 1.00 | 94.44 | A |
| ATOM | 2079 | CD | ARG | A | 317 | 7.668 | −.620 | 19.527 | .00 | 94.86 | A |
| ATOM | 2080 | NE | ARG | A | 317 | 8.881 | −.220 | 18.821 | .00 | 95.35 | A |
| ATOM | 2081 | CZ | ARG | A | 317 | 9.815 | −1.070 | 18.408 | .00 | 95.54 | A |
| ATOM | 2082 | NH1 | ARG | A | 317 | 9.676 | −2.369 | 18.632 | .00 | 95.67 | A |
| ATOM | 2083 | NH2 | ARG | A | 317 | 10.890 | −.622 | 17.773 | .00 | 95.62 | A |
| ATOM | 2084 | C | ARG | A | 317 | 4.577 | −1.086 | 18.917 | 1.00 | 93.38 | A |
| ATOM | 2085 | O | ARG | A | 317 | 4.099 | −.956 | 17.788 | 1.00 | 93.56 | A |
| ATOM | 2086 | N | GLY | A | 318 | 5.305 | −2.131 | 19.284 | 1.00 | 93.19 | A |
| ATOM | 2087 | CA | GLY | A | 318 | 5.582 | −3.207 | 18.355 | 1.00 | 92.86 | A |
| ATOM | 2088 | C | GLY | A | 318 | 5.181 | −4.502 | 19.018 | 1.00 | 92.67 | A |
| ATOM | 2089 | O | GLY | A | 318 | 4.855 | −5.485 | 18.351 | 1.00 | 93.44 | A |
| ATOM | 2090 | N | ASN | A | 319 | 5.208 | −4.496 | 20.346 | 1.00 | 92.07 | A |
| ATOM | 2091 | CA | ASN | A | 319 | 4.832 | −5.672 | 21.116 | 1.00 | 91.88 | A |
| ATOM | 2092 | CB | ASN | A | 319 | 5.677 | −5.778 | 22.393 | 1.00 | 93.88 | A |
| ATOM | 2093 | CG | ASN | A | 319 | 6.052 | −4.422 | 22.961 | 1.00 | 94.90 | A |
| ATOM | 2094 | OD1 | ASN | A | 319 | 6.962 | −3.754 | 22.457 | 1.00 | 95.24 | A |
| ATOM | 2095 | ND2 | ASN | A | 319 | 5.348 | −4.003 | 24.009 | 1.00 | 94.90 | A |
| ATOM | 2096 | C | ASN | A | 319 | 3.352 | −5.610 | 21.469 | 1.00 | 89.43 | A |
| ATOM | 2097 | O | ASN | A | 319 | 2.808 | −4.536 | 21.709 | 1.00 | 88.27 | A |
| ATOM | 2098 | N | PRO | A | 320 | 2.684 | −6.774 | 21.486 | 1.00 | 87.34 | A |
| ATOM | 2099 | CD | PRO | A | 320 | 3.248 | −8.091 | 21.136 | 1.00 | 87.45 | A |
| ATOM | 2100 | CA | PRO | A | 320 | 1.263 | −6.897 | 21.803 | 1.00 | 85.17 | A |
| ATOM | 2101 | CB | PRO | A | 320 | 1.085 | −8.405 | 21.956 | 1.00 | 86.24 | A |
| ATOM | 2102 | CG | PRO | A | 320 | 2.002 | −8.928 | 20.907 | 1.00 | 87.40 | A |
| ATOM | 2103 | C | PRO | A | 320 | .856 | −6.144 | 23.052 | 1.00 | 81.76 | A |
| ATOM | 2104 | O | PRO | A | 320 | 1.693 | −5.803 | 23.879 | 1.00 | 81.32 | A |
| ATOM | 2105 | N | PHE | A | 321 | −.434 | −5.856 | 23.158 | 1.00 | 78.17 | A |
| ATOM | 2106 | CA | PHE | A | 321 | −.979 | −5.181 | 24.323 | 1.00 | 75.27 | A |
| ATOM | 2107 | CB | PHE | A | 321 | −1.692 | −3.893 | 23.929 | 1.00 | 71.80 | A |
| ATOM | 2108 | CG | PHE | A | 321 | −2.159 | −3.093 | 25.101 | 1.00 | 69.03 | A |
| ATOM | 2109 | CD1 | PHE | A | 321 | −1.323 | −2.153 | 25.694 | 1.00 | 67.68 | A |
| ATOM | 2110 | CD2 | PHE | A | 321 | −3.417 | −3.317 | 25.653 | 1.00 | 67.28 | A |
| ATOM | 2111 | CE1 | PHE | A | 321 | −1.732 | −1.448 | 26.826 | 1.00 | 66.81 | A |
| ATOM | 2112 | CE2 | PHE | A | 321 | −3.835 | −2.618 | 26.786 | 1.00 | 66.90 | A |
| ATOM | 2113 | CZ | PHE | A | 321 | −2.990 | −1.682 | 27.373 | 1.00 | 66.54 | A |
| ATOM | 2114 | C | PHE | A | 321 | −1.990 | −6.183 | 24.865 | 1.00 | 75.48 | A |
| ATOM | 2115 | O | PHE | A | 321 | −3.108 | −6.274 | 24.361 | 1.00 | 75.77 | A |
| ATOM | 2116 | N | LEU | A | 322 | −1.598 | −6.940 | 25.883 | 1.00 | 74.77 | A |
| ATOM | 2117 | CA | LEU | A | 322 | −2.471 | −7.966 | 26.435 | 1.00 | 74.72 | A |
| ATOM | 2118 | CB | LEU | A | 322 | −1.653 | −8.931 | 27.287 | 1.00 | 74.98 | A |
| ATOM | 2119 | CG | LEU | A | 322 | −.425 | −9.481 | 26.562 | 1.00 | 76.20 | A |
| ATOM | 2120 | CD1 | LEU | A | 322 | .157 | −10.629 | 27.369 | 1.00 | 77.46 | A |
| ATOM | 2121 | CD2 | LEU | A | 322 | −.806 | −9.958 | 25.159 | 1.00 | 76.99 | A |
| ATOM | 2122 | C | LEU | A | 322 | −3.667 | −7.474 | 27.229 | 1.00 | 75.09 | A |
| ATOM | 2123 | O | LEU | A | 322 | −3.532 | −6.722 | 28.195 | 1.00 | 75.79 | A |
| ATOM | 2124 | N | LEU | A | 323 | −4.843 | −7.922 | 26.808 | 1.00 | 74.79 | A |
| ATOM | 2125 | CA | LEU | A | 323 | −6.093 | −7.563 | 27.453 | 1.00 | 75.00 | A |
| ATOM | 2126 | CB | LEU | A | 323 | −7.215 | −7.578 | 26.424 | 1.00 | 74.65 | A |
| ATOM | 2127 | CG | LEU | A | 323 | −7.409 | −6.316 | 25.578 | 1.00 | 75.10 | A |
| ATOM | 2128 | CD1 | LEU | A | 323 | −6.139 | −5.478 | 25.546 | 1.00 | 75.43 | A |
| ATOM | 2129 | CD2 | LEU | A | 323 | −7.845 | −6.727 | 24.179 | 1.00 | 74.55 | A |
| ATOM | 2130 | C | LEU | A | 323 | −6.431 | −8.510 | 28.593 | 1.00 | 75.84 | A |
| ATOM | 2131 | O | LEU | A | 323 | −7.560 | −8.544 | 29.061 | 1.00 | 75.91 | A |
| ATOM | 2132 | N | ASN | A | 324 | −5.445 | −9.276 | 29.040 | 1.00 | 76.74 | A |
| ATOM | 2133 | CA | ASN | A | 324 | −5.646 | −10.232 | 30.124 | 1.00 | 77.13 | A |
| ATOM | 2134 | CB | ASN | A | 324 | −4.289 | −10.698 | 30.666 | 1.00 | 79.72 | A |
| ATOM | 2135 | CG | ASN | A | 324 | −3.505 | −9.566 | 31.335 | 1.00 | 82.54 | A |
| ATOM | 2136 | OD1 | ASN | A | 324 | −3.265 | −8.512 | 30.730 | 1.00 | 83.61 | A |
| ATOM | 2137 | ND2 | ASN | A | 324 | −3.104 | −9.782 | 32.589 | 1.00 | 82.24 | A |
| ATOM | 2138 | C | ASN | A | 324 | −6.462 | −9.627 | 31.264 | 1.00 | 75.56 | A |
| ATOM | 2139 | O | ASN | A | 324 | −7.355 | −10.268 | 31.808 | 1.00 | 73.72 | A |
| ATOM | 2140 | N | PHE | A | 325 | −6.148 | −8.383 | 31.609 | 1.00 | 75.06 | A |
| ATOM | 2141 | CA | PHE | A | 325 | −6.821 | −7.690 | 32.701 | 1.00 | 75.56 | A |
| ATOM | 2142 | CB | PHE | A | 325 | −6.183 | −6.312 | 32.933 | 1.00 | 74.82 | A |
| ATOM | 2143 | CG | PHE | A | 325 | −6.098 | −5.455 | 31.702 | 1.00 | 74.16 | A |
| ATOM | 2144 | CD1 | PHE | A | 325 | −6.591 | −4.159 | 31.723 | 1.00 | 72.64 | A |
| ATOM | 2145 | CD2 | PHE | A | 325 | −5.515 | −5.933 | 30.533 | 1.00 | 74.40 | A |
| ATOM | 2146 | CE1 | PHE | A | 325 | −6.509 | −3.355 | 30.605 | 1.00 | 73.91 | A |
| ATOM | 2147 | CE2 | PHE | A | 325 | −5.428 | −5.131 | 29.402 | 1.00 | 74.58 | A |
| ATOM | 2148 | CZ | PHE | A | 325 | −5.925 | −3.842 | 29.435 | 1.00 | 74.68 | A |
| ATOM | 2149 | C | PHE | A | 325 | −8.338 | −7.549 | 32.574 | 1.00 | 75.87 | A |
| ATOM | 2150 | O | PHE | A | 325 | −9.028 | −7.324 | 33.573 | 1.00 | 75.26 | A |
| ATOM | 2151 | N | ILE | A | 326 | −8.852 | −7.693 | 31.355 | 1.00 | 75.81 | A |
| ATOM | 2152 | CA | ILE | A | 326 | −10.286 | −7.601 | 31.109 | 1.00 | 74.94 | A |
| ATOM | 2153 | CB | ILE | A | 326 | −10.599 | −7.722 | 29.608 | 1.00 | 76.10 | A |
| ATOM | 2154 | CG2 | ILE | A | 326 | −9.854 | −6.615 | 28.850 | 1.00 | 76.06 | A |
| ATOM | 2155 | CG1 | ILE | A | 326 | −10.201 | −9.122 | 29.081 | 1.00 | 77.00 | A |
| ATOM | 2156 | CD1 | ILE | A | 326 | −11.343 | −10.148 | 28.940 | 1.00 | 74.12 | A |
| ATOM | 2157 | C | ILE | A | 326 | −11.012 | −8.719 | 31.837 | 1.00 | 74.71 | A |
| ATOM | 2158 | O | ILE | A | 326 | −10.805 | −9.902 | 31.550 | 1.00 | 74.27 | A |
| ATOM | 2159 | N | PRO | A | 327 | −11.861 | −8.366 | 32.809 | 1.00 | 74.02 | A |
| ATOM | 2160 | CD | PRO | A | 327 | −12.115 | −7.046 | 33.399 | 1.00 | 72.80 | A |
| ATOM | 2161 | CA | PRO | A | 327 | −12.585 | −9.413 | 33.531 | 1.00 | 73.97 | A |
| ATOM | 2162 | CB | PRO | A | 327 | −13.332 | −8.634 | 34.601 | 1.00 | 73.72 | A |
| ATOM | 2163 | CG | PRO | A | 327 | −12.469 | −7.414 | 34.802 | 1.00 | 73.40 | A |
| ATOM | 2164 | C | PRO | A | 327 | −13.528 | −10.076 | 32.542 | 1.00 | 74.47 | A |
| ATOM | 2165 | O | PRO | A | 327 | −13.866 | −9.485 | 31.520 | 1.00 | 74.52 | A |
| ATOM | 2166 | N | GLU | A | 328 | −13.948 | −11.301 | 32.826 | 1.00 | 75.54 | A |
| ATOM | 2167 | CA | GLU | A | 328 | −14.858 | −11.999 | 31.915 | 1.00 | 76.21 | A |
| ATOM | 2168 | CB | GLU | A | 328 | −14.632 | −13.516 | 32.010 | 1.00 | 78.09 | A |
| ATOM | 2169 | CG | GLU | A | 328 | −15.401 | −14.354 | 31.001 | 1.00 | 79.85 | A |
| ATOM | 2170 | CD | GLU | A | 328 | −15.025 | −15.832 | 31.060 | 1.00 | 81.26 | A |
| ATOM | 2171 | OE1 | GLU | A | 328 | −15.120 | −16.434 | 32.154 | 1.00 | 80.99 | A |
| ATOM | 2172 | OE2 | GLU | A | 328 | −14.636 | −16.392 | 30.009 | 1.00 | 81.76 | A |
| ATOM | 2173 | C | GLU | A | 328 | −16.305 | −11.631 | 32.270 | 1.00 | 74.58 | A |
| ATOM | 2174 | O | GLU | A | 328 | −16.846 | −12.056 | 33.297 | 1.00 | 73.80 | A |
| ATOM | 2175 | N | VAL | A | 329 | −16.920 | −10.814 | 31.421 | 1.00 | 72.53 | A |
| ATOM | 2176 | CA | VAL | A | 329 | −18.286 | −10.382 | 31.664 | 1.00 | 69.58 | A |
| ATOM | 2177 | CB | VAL | A | 329 | −18.562 | −9.000 | 31.021 | 1.00 | 69.40 | A |
| ATOM | 2178 | CG1 | VAL | A | 329 | −19.982 | −8.556 | 31.336 | 1.00 | 69.60 | A |
| ATOM | 2179 | CG2 | VAL | A | 329 | −17.574 | −7.976 | 31.547 | 1.00 | 68.88 | A |
| ATOM | 2180 | C | VAL | A | 329 | −19.256 | −11.403 | 31.100 | 1.00 | 66.80 | A |
| ATOM | 2181 | O | VAL | A | 329 | −19.067 | −11.919 | 30.006 | 1.00 | 66.58 | A |
| ATOM | 2182 | N | ASP | A | 330 | −20.300 | −11.700 | 31.853 | 1.00 | 63.62 | A |
| ATOM | 2183 | CA | ASP | A | 330 | −21.271 | −12.662 | 31.391 | 1.00 | 62.48 | A |
| ATOM | 2184 | CB | ASP | A | 330 | −21.712 | −13.529 | 32.572 | 1.00 | 65.77 | A |
| ATOM | 2185 | CG | ASP | A | 330 | −20.528 | −14.243 | 33.245 | 1.00 | 68.23 | A |
| ATOM | 2186 | OD1 | ASP | A | 330 | −19.765 | −14.952 | 32.541 | 1.00 | 68.53 | A |
| ATOM | 2187 | OD2 | ASP | A | 330 | −20.357 | −14.096 | 34.478 | 1.00 | 69.45 | A |
| ATOM | 2188 | C | ASP | A | 330 | −22.450 | −11.954 | 30.723 | 1.00 | 59.67 | A |
| ATOM | 2189 | O | ASP | A | 330 | −22.571 | −10.742 | 30.803 | 1.00 | 59.34 | A |
| ATOM | 2190 | N | TYR | A | 331 | −23.311 | −12.697 | 30.042 | 1.00 | 57.01 | A |
| ATOM | 2191 | CA | TYR | A | 331 | −24.444 | −12.068 | 29.367 | 1.00 | 54.42 | A |
| ATOM | 2192 | CB | TYR | A | 331 | −24.186 | −11.974 | 27.853 | 1.00 | 51.28 | A |
| ATOM | 2193 | CG | TYR | A | 331 | −23.019 | −11.105 | 27.494 | 1.00 | 48.96 | A |
| ATOM | 2194 | CD1 | TYR | A | 331 | −21.722 | −11.600 | 27.535 | 1.00 | 47.89 | A |
| ATOM | 2195 | CE1 | TYR | A | 331 | −20.641 | −10.770 | 27.294 | 1.00 | 47.64 | A |
| ATOM | 2196 | CD2 | TYR | A | 331 | −23.204 | −9.757 | 27.192 | 1.00 | 47.80 | A |
| ATOM | 2197 | CE2 | TYR | A | 331 | −22.128 | −8.915 | 26.949 | 1.00 | 45.25 | A |
| ATOM | 2198 | CZ | TYR | A | 331 | −20.854 | −9.425 | 27.007 | 1.00 | 46.15 | A |
| ATOM | 2199 | OH | TYR | A | 331 | −19.783 | −8.590 | 26.821 | 1.00 | 46.94 | A |
| ATOM | 2200 | C | TYR | A | 331 | −25.778 | −12.769 | 29.572 | 1.00 | 53.85 | A |
| ATOM | 2201 | O | TYR | A | 331 | −25.836 | −13.970 | 29.855 | 1.00 | 55.22 | A |
| ATOM | 2202 | N | VAL | A | 332 | −26.853 | −12.006 | 29.422 | 1.00 | 52.05 | A |
| ATOM | 2203 | CA | VAL | A | 332 | −28.194 | −12.560 | 29.532 | 1.00 | 51.21 | A |
| ATOM | 2204 | CB | VAL | A | 332 | −29.222 | −11.520 | 30.055 | 1.00 | 51.94 | A |
| ATOM | 2205 | CG1 | VAL | A | 332 | −30.591 | −12.155 | 30.153 | 1.00 | 49.43 | A |
| ATOM | 2206 | CG2 | VAL | A | 332 | −28.802 | −10.987 | 31.405 | 1.00 | 52.51 | A |
| ATOM | 2207 | C | VAL | A | 332 | −28.623 | −12.976 | 28.124 | 1.00 | 49.69 | A |
| ATOM | 2208 | O | VAL | A | 332 | −28.809 | −12.139 | 27.245 | 1.00 | 49.59 | A |
| ATOM | 2209 | N | PRO | A | 333 | −28.764 | −14.277 | 27.892 | 1.00 | 48.89 | A |
| ATOM | 2210 | CD | PRO | A | 333 | −28.333 | −15.354 | 28.797 | 1.00 | 50.73 | A |
| ATOM | 2211 | CA | PRO | A | 333 | −29.171 | −14.829 | 26.603 | 1.00 | 49.60 | A |
| ATOM | 2212 | CB | PRO | A | 333 | −28.545 | −16.206 | 26.628 | 1.00 | 51.00 | A |
| ATOM | 2213 | CG | PRO | A | 333 | −28.778 | −16.607 | 28.059 | 1.00 | 50.16 | A |
| ATOM | 2214 | C | PRO | A | 333 | −30.684 | −14.919 | 26.555 | 1.00 | 50.29 | A |
| ATOM | 2215 | O | PRO | A | 333 | −31.317 | −15.274 | 27.554 | 1.00 | 51.63 | A |
| ATOM | 2216 | N | LEU | A | 334 | −31.271 | −14.609 | 25.406 | 1.00 | 49.52 | A |
| ATOM | 2217 | CA | LEU | A | 334 | −32.720 | −14.677 | 25.283 | 1.00 | 48.88 | A |
| ATOM | 2218 | CB | LEU | A | 334 | −33.377 | −13.604 | 26.175 | 1.00 | 50.88 | A |
| ATOM | 2219 | CG | LEU | A | 334 | −33.818 | −12.217 | 25.673 | 1.00 | 50.95 | A |
| ATOM | 2220 | CD1 | LEU | A | 334 | −33.872 | −11.274 | 26.851 | 1.00 | 49.49 | A |
| ATOM | 2221 | CD2 | LEU | A | 334 | −32.857 | −11.664 | 24.672 | 1.00 | 51.98 | A |
| ATOM | 2222 | C | LEU | A | 334 | −33.132 | −14.494 | 23.842 | 1.00 | 46.70 | A |
| ATOM | 2223 | O | LEU | A | 334 | −32.387 | −13.973 | 23.038 | 1.00 | 46.82 | A |
| ATOM | 2224 | N | SER | A | 335 | −34.318 | −14.939 | 23.498 | 1.00 | 45.88 | A |
| ATOM | 2225 | CA | SER | A | 335 | −34.743 | −14.761 | 22.133 | 1.00 | 46.96 | A |
| ATOM | 2226 | CB | SER | A | 335 | −34.818 | −16.118 | 21.421 | 1.00 | 48.12 | A |
| ATOM | 2227 | OG | SER | A | 335 | −35.664 | −17.012 | 22.113 | 1.00 | 50.68 | A |
| ATOM | 2228 | C | SER | A | 335 | −36.090 | −14.058 | 22.120 | 1.00 | 45.84 | A |
| ATOM | 2229 | O | SER | A | 335 | −36.895 | −14.236 | 23.040 | 1.00 | 47.10 | A |
| ATOM | 2230 | N | VAL | A | 336 | −36.314 | −13.236 | 21.102 | 1.00 | 41.82 | A |
| ATOM | 2231 | CA | VAL | A | 336 | −37.572 | −12.528 | 20.966 | 1.00 | 41.34 | A |
| ATOM | 2232 | CB | VAL | A | 336 | −37.357 | −11.159 | 20.316 | 1.00 | 41.49 | A |
| ATOM | 2233 | CG1 | VAL | A | 336 | −38.703 | −10.511 | 19.952 | 1.00 | 38.96 | A |
| ATOM | 2234 | CG2 | VAL | A | 336 | −36.572 | −10.282 | 21.259 | 1.00 | 41.60 | A |
| ATOM | 2235 | C | VAL | A | 336 | −38.498 | −13.340 | 20.078 | 1.00 | 41.66 | A |
| ATOM | 2236 | O | VAL | A | 336 | −38.195 | −13.560 | 18.910 | 1.00 | 42.87 | A |
| ATOM | 2237 | N | VAL | A | 337 | −39.620 | −13.791 | 20.619 | 1.00 | 41.10 | A |
| ATOM | 2238 | CA | VAL | A | 337 | −40.561 | −14.570 | 19.822 | 1.00 | 43.03 | A |
| ATOM | 2239 | CB | VAL | A | 337 | −41.020 | −15.838 | 20.559 | 1.00 | 42.70 | A |
| ATOM | 2240 | CG1 | VAL | A | 337 | −42.133 | −16.505 | 19.782 | 1.00 | 40.10 | A |
| ATOM | 2241 | CG2 | VAL | A | 337 | −39.834 | −16.790 | 20.745 | 1.00 | 42.76 | A |
| ATOM | 2242 | C | VAL | A | 337 | −41.796 | −13.771 | 19.443 | 1.00 | 44.88 | A |
| ATOM | 2243 | O | VAL | A | 337 | −42.381 | −13.083 | 20.275 | 1.00 | 46.00 | A |
| ATOM | 2244 | N | ILE | A | 338 | −42.186 | −13.854 | 18.177 | 1.00 | 47.42 | A |
| ATOM | 2245 | CA | ILE | A | 338 | −43.371 | −13.146 | 17.693 | 1.00 | 48.81 | A |
| ATOM | 2246 | CB | ILE | A | 338 | −43.055 | −12.287 | 16.439 | 1.00 | 49.56 | A |
| ATOM | 2247 | CG2 | ILE | A | 338 | −44.280 | −12.179 | 15.555 | 1.00 | 51.04 | A |
| ATOM | 2248 | CG1 | ILE | A | 338 | −42.634 | −10.878 | 16.848 | 1.00 | 49.20 | A |
| ATOM | 2249 | CD1 | ILE | A | 338 | −41.434 | −10.832 | 17.729 | 1.00 | 50.30 | A |
| ATOM | 2250 | C | ILE | A | 338 | −44.464 | −14.149 | 17.340 | 1.00 | 49.08 | A |
| ATOM | 2251 | O | ILE | A | 338 | −44.200 | −15.175 | 16.702 | 1.00 | 49.86 | A |
| ATOM | 2252 | N | THR | A | 339 | −45.682 | −13.863 | 17.785 | 1.00 | 48.44 | A |
| ATOM | 2253 | CA | THR | A | 339 | −46.824 | −14.714 | 17.495 | 1.00 | 49.34 | A |
| ATOM | 2254 | CB | THR | A | 339 | −47.222 | −15.614 | 18.670 | 1.00 | 49.60 | A |
| ATOM | 2255 | OG1 | THR | A | 339 | −47.641 | −14.791 | 19.764 | 1.00 | 52.16 | A |
| ATOM | 2256 | CG2 | THR | A | 339 | −46.077 | −16.512 | 19.083 | 1.00 | 46.94 | A |
| ATOM | 2257 | C | THR | A | 339 | −48.000 | −13.814 | 17.214 | 1.00 | 49.87 | A |
| ATOM | 2258 | O | THR | A | 339 | −48.004 | −12.641 | 17.581 | 1.00 | 49.38 | A |
| ATOM | 2259 | N | THR | A | 340 | −49.021 | −14.374 | 16.582 | 1.00 | 52.58 | A |
| ATOM | 2260 | CA | THR | A | 340 | −50.197 | −13.586 | 16.241 | 1.00 | 54.78 | A |
| ATOM | 2261 | CB | THR | A | 340 | −50.038 | −13.041 | 14.789 | 1.00 | 54.95 | A |
| ATOM | 2262 | OG1 | THR | A | 340 | −50.859 | −11.885 | 14.612 | 1.00 | 56.39 | A |
| ATOM | 2263 | CG2 | THR | A | 340 | −50.406 | −14.102 | 13.763 | 1.00 | 53.48 | A |
| ATOM | 2264 | C | THR | A | 340 | −51.479 | −14.408 | 16.371 | 1.00 | 55.17 | A |
| ATOM | 2265 | O | THR | A | 340 | −51.637 | −15.408 | 15.698 | 1.00 | 56.61 | A |
| ATOM | 2266 | N | PHE | A | 341 | −52.393 | −14.014 | 17.248 | 1.00 | 55.58 | A |
| ATOM | 2267 | CA | PHE | A | 341 | −53.635 | −14.782 | 17.369 | 1.00 | 56.53 | A |
| ATOM | 2268 | CB | PHE | A | 341 | −53.905 | −15.191 | 18.821 | 1.00 | 53.42 | A |
| ATOM | 2269 | CG | PHE | A | 341 | −52.916 | −16.177 | 19.376 | 1.00 | 52.38 | A |
| ATOM | 2270 | CD1 | PHE | A | 341 | −51.579 | −15.818 | 19.563 | 1.00 | 51.93 | A |
| ATOM | 2271 | CD2 | PHE | A | 341 | −53.325 | −17.461 | 19.746 | 1.00 | 50.15 | A |
| ATOM | 2272 | CE1 | PHE | A | 341 | −50.658 | −16.725 | 20.118 | 1.00 | 51.53 | A |
| ATOM | 2273 | CE2 | PHE | A | 341 | −52.419 | −18.374 | 20.299 | 1.00 | 49.24 | A |
| ATOM | 2274 | CZ | PHE | A | 341 | −51.081 | −18.008 | 20.488 | 1.00 | 49.75 | A |
| ATOM | 2275 | C | PHE | A | 341 | −54.822 | −13.984 | 16.852 | 1.00 | 58.01 | A |
| ATOM | 2276 | O | PHE | A | 341 | −54.846 | −12.751 | 16.958 | 1.00 | 56.86 | A |
| ATOM | 2277 | N | LYS | A | 342 | −55.800 | −14.682 | 16.278 | 1.00 | 60.09 | A |
| ATOM | 2278 | CA | LYS | A | 342 | −56.992 | −14.001 | 15.784 | 1.00 | 62.17 | A |
| ATOM | 2279 | CB | LYS | A | 342 | −57.876 | −14.959 | 14.974 | 1.00 | 62.77 | A |
| ATOM | 2280 | CG | LYS | A | 342 | −57.877 | −14.652 | 13.468 | 1.00 | 66.87 | A |
| ATOM | 2281 | CD | LYS | A | 342 | −58.644 | −15.698 | 12.644 | 1.00 | 68.85 | A |
| ATOM | 2282 | CE | LYS | A | 342 | −57.972 | −17.079 | 12.717 | 1.00 | 69.94 | A |
| ATOM | 2283 | NZ | LYS | A | 342 | −58.761 | −18.167 | 12.061 | 1.00 | 69.13 | A |
| ATOM | 2284 | C | LYS | A | 342 | −57.719 | −13.519 | 17.024 | 1.00 | 61.94 | A |
| ATOM | 2285 | O | LYS | A | 342 | −57.901 | −14.275 | 17.970 | 1.00 | 60.22 | A |
| ATOM | 2286 | N | ARG | A | 343 | −58.120 | −12.257 | 17.035 | 1.00 | 64.28 | A |
| ATOM | 2287 | CA | ARG | A | 343 | −58.801 | −11.719 | 18.205 | 1.00 | 67.57 | A |
| ATOM | 2288 | CB | ARG | A | 343 | −59.361 | −10.328 | 17.904 | 1.00 | 66.70 | A |
| ATOM | 2289 | CG | ARG | A | 343 | −58.267 | −9.335 | 17.583 | 1.00 | 66.47 | A |
| ATOM | 2290 | CD | ARG | A | 343 | −58.676 | −7.899 | 17.794 | 1.00 | 67.35 | A |
| ATOM | 2291 | NE | ARG | A | 343 | −57.532 | −7.028 | 17.532 | 1.00 | 70.33 | A |
| ATOM | 2292 | CZ | ARG | A | 343 | −57.556 | −5.699 | 17.596 | 1.00 | 70.86 | A |
| ATOM | 2293 | NH1 | ARG | A | 343 | −58.678 | −5.067 | 17.918 | 1.00 | 69.70 | A |
| ATOM | 2294 | NH2 | ARG | A | 343 | −56.451 | −5.004 | 17.331 | 1.00 | 70.86 | A |
| ATOM | 2295 | C | ARG | A | 343 | −59.899 | −12.630 | 18.722 | 1.00 | 70.28 | A |
| ATOM | 2296 | O | ARG | A | 343 | −60.092 | −12.759 | 19.929 | 1.00 | 71.38 | A |
| ATOM | 2297 | N | GLU | A | 344 | −60.608 | −13.280 | 17.810 | 1.00 | 73.59 | A |
| ATOM | 2298 | CA | GLU | A | 344 | −61.687 | −14.182 | 18.198 | 1.00 | 75.93 | A |
| ATOM | 2299 | CB | GLU | A | 344 | −62.374 | −14.758 | 16.945 | 1.00 | 79.00 | A |
| ATOM | 2300 | CG | GLU | A | 344 | −63.074 | −13.713 | 16.088 | 1.00 | 84.24 | A |
| ATOM | 2301 | CD | GLU | A | 344 | −64.023 | −14.307 | 15.055 | 1.00 | 87.26 | A |
| ATOM | 2302 | OE1 | GLU | A | 344 | −63.544 | −14.960 | 14.099 | 1.00 | 89.32 | A |
| ATOM | 2303 | OE2 | GLU | A | 344 | −65.253 | −14.115 | 15.202 | 1.00 | 88.34 | A |
| ATOM | 2304 | C | GLU | A | 344 | −61.242 | −15.333 | 19.107 | 1.00 | 75.13 | A |
| ATOM | 2305 | O | GLU | A | 344 | −62.081 | −16.017 | 19.688 | 1.00 | 76.59 | A |
| ATOM | 2306 | N | ASN | A | 345 | −59.937 | −15.552 | 19.243 | 1.00 | 73.81 | A |
| ATOM | 2307 | CA | ASN | A | 345 | −59.465 | −16.657 | 20.075 | 1.00 | 72.73 | A |
| ATOM | 2308 | CB | ASN | A | 345 | −58.574 | −17.589 | 19.254 | 1.00 | 73.33 | A |
| ATOM | 2309 | CG | ASN | A | 345 | −59.318 | −18.231 | 18.106 | 1.00 | 72.74 | A |
| ATOM | 2310 | OD1 | ASN | A | 345 | −60.411 | −18.760 | 18.291 | 1.00 | 72.60 | A |
| ATOM | 2311 | ND2 | ASN | A | 345 | −58.727 | −18.197 | 16.916 | 1.00 | 71.84 | A |
| ATOM | 2312 | C | ASN | A | 345 | −58.742 | −16.265 | 21.360 | 1.00 | 71.56 | A |
| ATOM | 2313 | O | ASN | A | 345 | −58.242 | −17.128 | 22.085 | 1.00 | 71.50 | A |
| ATOM | 2314 | N | VAL | A | 346 | −58.681 | −14.969 | 21.638 | 1.00 | 69.39 | A |
| ATOM | 2315 | CA | VAL | A | 346 | −58.042 | −14.489 | 22.856 | 1.00 | 67.76 | A |
| ATOM | 2316 | CB | VAL | A | 346 | −57.200 | −13.220 | 22.583 | 1.00 | 69.25 | A |
| ATOM | 2317 | CG1 | VAL | A | 346 | −56.823 | −12.546 | 23.899 | 1.00 | 68.94 | A |
| ATOM | 2318 | CG2 | VAL | A | 346 | −55.933 | −13.598 | 21.801 | 1.00 | 68.54 | A |
| ATOM | 2319 | C | VAL | A | 346 | −59.145 | −14.186 | 23.869 | 1.00 | 65.93 | A |
| ATOM | 2320 | O | VAL | A | 346 | −59.691 | −13.087 | 23.918 | 1.00 | 64.76 | A |
| ATOM | 2321 | N | LYS | A | 347 | −59.469 | −15.190 | 24.673 | 1.00 | 64.71 | A |
| ATOM | 2322 | CA | LYS | A | 347 | −60.520 | −15.086 | 25.678 | 1.00 | 63.44 | A |
| ATOM | 2323 | CB | LYS | A | 347 | −60.627 | −16.403 | 26.441 | 1.00 | 63.72 | A |
| ATOM | 2324 | CG | LYS | A | 347 | −61.173 | −17.533 | 25.599 | 1.00 | 65.67 | A |
| ATOM | 2325 | CD | LYS | A | 347 | −60.943 | −18.883 | 26.255 | 1.00 | 68.55 | A |
| ATOM | 2326 | CE | LYS | A | 347 | −61.570 | −18.964 | 27.637 | 1.00 | 69.63 | A |
| ATOM | 2327 | NZ | LYS | A | 347 | −61.270 | −20.276 | 28.287 | 1.00 | 71.94 | A |
| ATOM | 2328 | C | LYS | A | 347 | −60.431 | −13.950 | 26.682 | 1.00 | 61.43 | A |
| ATOM | 2329 | O | LYS | A | 347 | −61.415 | −13.251 | 26.908 | 1.00 | 60.17 | A |
| ATOM | 2330 | N | TYR | A | 348 | −59.266 | −13.770 | 27.294 | 1.00 | 59.89 | A |
| ATOM | 2331 | CA | TYR | A | 348 | −59.111 | −12.727 | 28.295 | 1.00 | 58.22 | A |
| ATOM | 2332 | CB | TYR | A | 348 | −58.799 | −13.378 | 29.635 | 1.00 | 57.87 | A |
| ATOM | 2333 | CG | TYR | A | 348 | −59.726 | −14.531 | 29.979 | 1.00 | 57.73 | A |
| ATOM | 2334 | CD1 | TYR | A | 348 | −59.230 | −15.820 | 30.164 | 1.00 | 59.10 | A |
| ATOM | 2335 | CE1 | TYR | A | 348 | −60.077 | −16.888 | 30.502 | 1.00 | 57.47 | A |
| ATOM | 2336 | CD2 | TYR | A | 348 | −61.092 | −14.332 | 30.138 | 1.00 | 58.25 | A |
| ATOM | 2337 | CE2 | TYR | A | 348 | −61.950 | −15.389 | 30.478 | 1.00 | 57.77 | A |
| ATOM | 2338 | CZ | TYR | A | 348 | −61.439 | −16.660 | 30.657 | 1.00 | 58.14 | A |
| ATOM | 2339 | OH | TYR | A | 348 | −62.301 | −17.691 | 30.977 | 1.00 | 56.50 | A |
| ATOM | 2340 | C | TYR | A | 348 | −58.055 | −11.674 | 27.963 | 1.00 | 56.88 | A |
| ATOM | 2341 | O | TYR | A | 348 | −56.970 | −11.677 | 28.528 | 1.00 | 56.64 | A |
| ATOM | 2342 | N | PRO | A | 349 | −58.370 | −10.744 | 27.050 | 1.00 | 56.55 | A |
| ATOM | 2343 | CD | PRO | A | 349 | −59.575 | −10.604 | 26.221 | 1.00 | 55.90 | A |
| ATOM | 2344 | CA | PRO | A | 349 | −57.391 | −9.713 | 26.699 | 1.00 | 56.86 | A |
| ATOM | 2345 | CB | PRO | A | 349 | −58.026 | −9.006 | 25.494 | 1.00 | 55.24 | A |
| ATOM | 2346 | CG | PRO | A | 349 | −59.002 | −9.992 | 24.972 | 1.00 | 56.74 | A |
| ATOM | 2347 | C | PRO | A | 349 | −57.195 | −8.739 | 27.850 | 1.00 | 56.95 | A |
| ATOM | 2348 | O | PRO | A | 349 | −58.059 | −8.604 | 28.726 | 1.00 | 58.07 | A |
| ATOM | 2349 | N | LEU | A | 350 | −56.060 | −8.057 | 27.840 | 1.00 | 55.22 | A |
| ATOM | 2350 | CA | LEU | A | 350 | −55.785 | −7.065 | 28.849 | 1.00 | 54.28 | A |
| ATOM | 2351 | CB | LEU | A | 350 | −54.681 | −7.550 | 29.787 | 1.00 | 54.06 | A |
| ATOM | 2352 | CG | LEU | A | 350 | −55.090 | −8.604 | 30.821 | 1.00 | 54.18 | A |
| ATOM | 2353 | CD1 | LEU | A | 350 | −53.845 | −9.101 | 31.525 | 1.00 | 53.93 | A |
| ATOM | 2354 | CD2 | LEU | A | 350 | −56.091 | −8.018 | 31.831 | 1.00 | 51.69 | A |
| ATOM | 2355 | C | LEU | A | 350 | −55.378 | −5.788 | 28.137 | 1.00 | 53.65 | A |
| ATOM | 2356 | O | LEU | A | 350 | −54.721 | −5.833 | 27.107 | 1.00 | 53.22 | A |
| ATOM | 2357 | N | GLU | A | 351 | −55.793 | −4.652 | 28.679 | 1.00 | 54.23 | A |
| ATOM | 2358 | CA | GLU | A | 351 | −55.462 | −3.358 | 28.103 | 1.00 | 54.95 | A |
| ATOM | 2359 | CB | GLU | A | 351 | −56.612 | −2.370 | 28.349 | 1.00 | 55.23 | A |
| ATOM | 2360 | CG | GLU | A | 351 | −56.481 | −1.042 | 27.604 | 1.00 | 56.44 | A |
| ATOM | 2361 | CD | GLU | A | 351 | −56.437 | −1.226 | 26.099 | .00 | 56.23 | A |
| ATOM | 2362 | OE1 | GLU | A | 351 | −57.418 | −1.756 | 25.536 | .00 | 56.37 | A |
| ATOM | 2363 | OE2 | GLU | A | 351 | −55.422 | −.843 | 25.479 | .00 | 56.37 | A |
| ATOM | 2364 | C | GLU | A | 351 | −54.176 | −2.835 | 28.747 | 1.00 | 54.90 | A |
| ATOM | 2365 | O | GLU | A | 351 | −54.020 | −2.902 | 29.964 | 1.00 | 55.65 | A |
| ATOM | 2366 | N | GLY | A | 352 | −53.259 | −2.321 | 27.931 | 1.00 | 55.22 | A |
| ATOM | 2367 | CA | GLY | A | 352 | −52.007 | −1.798 | 28.458 | 1.00 | 54.39 | A |
| ATOM | 2368 | C | GLY | A | 352 | −50.817 | −2.073 | 27.558 | 1.00 | 53.97 | A |
| ATOM | 2369 | O | GLY | A | 352 | −50.983 | −2.261 | 26.355 | 1.00 | 54.64 | A |
| ATOM | 2370 | N | PHE | A | 353 | −49.616 | −2.105 | 28.133 | 1.00 | 52.49 | A |
| ATOM | 2371 | CA | PHE | A | 353 | −48.411 | −2.357 | 27.347 | 1.00 | 49.89 | A |
| ATOM | 2372 | CB | PHE | A | 353 | −47.245 | −1.493 | 27.830 | 1.00 | 49.07 | A |
| ATOM | 2373 | CG | PHE | A | 353 | −47.422 | −.023 | 27.582 | 1.00 | 48.59 | A |
| ATOM | 2374 | CD1 | PHE | A | 353 | −48.605 | .479 | 27.047 | 1.00 | 50.09 | A |
| ATOM | 2375 | CD2 | PHE | A | 353 | −46.413 | .872 | 27.925 | 1.00 | 49.70 | A |
| ATOM | 2376 | CE1 | PHE | A | 353 | −48.784 | 1.853 | 26.864 | 1.00 | 50.20 | A |
| ATOM | 2377 | CE2 | PHE | A | 353 | −46.579 | 2.249 | 27.746 | 1.00 | 49.27 | A |
| ATOM | 2378 | CZ | PHE | A | 353 | −47.767 | 2.737 | 27.217 | 1.00 | 49.99 | A |
| ATOM | 2379 | C | PHE | A | 353 | −47.991 | −3.812 | 27.389 | 1.00 | 48.95 | A |
| ATOM | 2380 | O | PHE | A | 353 | −47.392 | −4.305 | 26.439 | 1.00 | 49.49 | A |
| ATOM | 2381 | N | GLY | A | 354 | −48.283 | −4.500 | 28.487 | 1.00 | 47.97 | A |
| ATOM | 2382 | CA | GLY | A | 354 | −47.912 | −5.903 | 28.564 | 1.00 | 48.37 | A |
| ATOM | 2383 | C | GLY | A | 354 | −47.994 | −6.580 | 29.926 | 1.00 | 49.26 | A |
| ATOM | 2384 | O | GLY | A | 354 | −48.711 | −6.138 | 30.826 | 1.00 | 48.86 | A |
| ATOM | 2385 | N | VAL | A | 355 | −47.245 | −7.673 | 30.062 | 1.00 | 49.42 | A |
| ATOM | 2386 | CA | VAL | A | 355 | −47.200 | −8.465 | 31.281 | 1.00 | 48.57 | A |
| ATOM | 2387 | CB | VAL | A | 355 | −48.057 | −9.729 | 31.156 | 1.00 | 49.15 | A |
| ATOM | 2388 | CG1 | VAL | A | 355 | −48.053 | −10.481 | 32.470 | 1.00 | 50.00 | A |
| ATOM | 2389 | CG2 | VAL | A | 355 | −49.458 | −9.372 | 30.755 | 1.00 | 49.74 | A |
| ATOM | 2390 | C | VAL | A | 355 | −45.780 | −8.937 | 31.550 | 1.00 | 49.02 | A |
| ATOM | 2391 | O | VAL | A | 355 | −45.112 | −9.447 | 30.653 | 1.00 | 50.75 | A |
| ATOM | 2392 | N | LEU | A | 356 | −45.328 | −8.782 | 32.788 | 1.00 | 47.92 | A |
| ATOM | 2393 | CA | LEU | A | 356 | −43.999 | −9.227 | 33.182 | 1.00 | 47.26 | A |
| ATOM | 2394 | CB | LEU | A | 356 | −43.342 | −8.159 | 34.026 | 1.00 | 48.09 | A |
| ATOM | 2395 | CG | LEU | A | 356 | −43.015 | −6.878 | 33.297 | 1.00 | 47.50 | A |
| ATOM | 2396 | CD1 | LEU | A | 356 | −42.651 | −5.825 | 34.303 | 1.00 | 49.11 | A |
| ATOM | 2397 | CD2 | LEU | A | 356 | −41.876 | −7.134 | 32.347 | 1.00 | 47.83 | A |
| ATOM | 2398 | C | LEU | A | 356 | −44.160 | −10.496 | 34.011 | 1.00 | 47.56 | A |
| ATOM | 2399 | O | LEU | A | 356 | −45.090 | −10.598 | 34.804 | 1.00 | 48.71 | A |
| ATOM | 2400 | N | VAL | A | 357 | −43.259 | −11.457 | 33.847 | 1.00 | 47.04 | A |
| ATOM | 2401 | CA | VAL | A | 357 | −43.360 | −12.705 | 34.598 | 1.00 | 46.37 | A |
| ATOM | 2402 | CB | VAL | A | 357 | −43.314 | −13.936 | 33.652 | 1.00 | 46.87 | A |
| ATOM | 2403 | CG1 | VAL | A | 357 | −43.315 | −15.217 | 34.454 | 1.00 | 45.17 | A |
| ATOM | 2404 | CG2 | VAL | A | 357 | −44.512 | −13.919 | 32.722 | 1.00 | 44.47 | A |
| ATOM | 2405 | C | VAL | A | 357 | −42.259 | −12.847 | 35.635 | 1.00 | 46.98 | A |
| ATOM | 2406 | O | VAL | A | 357 | −41.115 | −13.135 | 35.294 | 1.00 | 47.30 | A |
| ATOM | 2407 | N | PRO | A | 358 | −42.594 | −12.638 | 36.923 | 1.00 | 48.00 | A |
| ATOM | 2408 | CD | PRO | A | 358 | −43.932 | −12.295 | 37.437 | 1.00 | 47.94 | A |
| ATOM | 2409 | CA | PRO | A | 358 | −41.635 | −12.745 | 38.027 | 1.00 | 47.36 | A |
| ATOM | 2410 | CB | PRO | A | 358 | −42.501 | −12.508 | 39.256 | 1.00 | 46.21 | A |
| ATOM | 2411 | CG | PRO | A | 358 | −43.603 | −11.667 | 38.756 | 1.00 | 45.92 | A |
| ATOM | 2412 | C | PRO | A | 358 | −41.022 | −14.144 | 38.039 | 1.00 | 49.12 | A |
| ATOM | 2413 | O | PRO | A | 358 | −41.744 | −15.135 | 38.048 | 1.00 | 48.23 | A |
| ATOM | 2414 | N | SER | A | 359 | −39.696 | −14.224 | 38.044 | 1.00 | 51.42 | A |
| ATOM | 2415 | CA | SER | A | 359 | −39.005 | −15.508 | 38.042 | 1.00 | 53.33 | A |
| ATOM | 2416 | CB | SER | A | 359 | −37.547 | −15.309 | 38.405 | 1.00 | 53.05 | A |
| ATOM | 2417 | OG | SER | A | 359 | −37.464 | −14.598 | 39.618 | 1.00 | 57.38 | A |
| ATOM | 2418 | C | SER | A | 359 | −39.623 | −16.516 | 38.998 | 1.00 | 55.45 | A |
| ATOM | 2419 | O | SER | A | 359 | −39.527 | −17.725 | 38.785 | 1.00 | 57.22 | A |
| ATOM | 2420 | N | LYS | A | 360 | −40.257 | −16.022 | 40.055 | 1.00 | 57.46 | A |
| ATOM | 2421 | CA | LYS | A | 360 | −40.891 | −16.900 | 41.032 | 1.00 | 57.19 | A |
| ATOM | 2422 | CB | LYS | A | 360 | −41.322 | −16.103 | 42.268 | 1.00 | 58.45 | A |
| ATOM | 2423 | CG | LYS | A | 360 | −41.950 | −16.957 | 43.343 | 1.00 | 62.62 | A |
| ATOM | 2424 | CD | LYS | A | 360 | −41.919 | −16.274 | 44.693 | 1.00 | 66.88 | A |
| ATOM | 2425 | CE | LYS | A | 360 | −42.340 | −17.234 | 45.812 | 1.00 | 70.44 | A |
| ATOM | 2426 | NZ | LYS | A | 360 | −42.254 | −16.619 | 47.182 | 1.00 | 72.16 | A |
| ATOM | 2427 | C | LYS | A | 360 | −42.103 | −17.587 | 40.418 | 1.00 | 56.27 | A |
| ATOM | 2428 | O | LYS | A | 360 | −42.543 | −18.620 | 40.896 | 1.00 | 57.17 | A |
| ATOM | 2429 | N | GLU | A | 361 | −42.640 | −17.019 | 39.349 | 1.00 | 54.22 | A |
| ATOM | 2430 | CA | GLU | A | 361 | −43.802 | −17.609 | 38.726 | 1.00 | 52.31 | A |
| ATOM | 2431 | CB | GLU | A | 361 | −44.512 | −16.569 | 37.867 | 1.00 | 51.84 | A |
| ATOM | 2432 | CG | GLU | A | 361 | −45.216 | −15.535 | 38.692 | 1.00 | 53.25 | A |
| ATOM | 2433 | CD | GLU | A | 361 | −46.305 | −16.138 | 39.554 | 1.00 | 55.30 | A |
| ATOM | 2434 | OE1 | GLU | A | 361 | −47.289 | −16.646 | 38.982 | 1.00 | 55.98 | A |
| ATOM | 2435 | OE2 | GLU | A | 361 | −46.182 | −16.112 | 40.801 | 1.00 | 56.47 | A |
| ATOM | 2436 | C | GLU | A | 361 | −43.502 | −18.868 | 37.926 | 1.00 | 51.59 | A |
| ATOM | 2437 | O | GLU | A | 361 | −44.400 | −19.462 | 37.325 | 1.00 | 52.53 | A |
| ATOM | 2438 | N | GLN | A | 362 | −42.248 | −19.291 | 37.901 | 1.00 | 50.24 | A |
| ATOM | 2439 | CA | GLN | A | 362 | −41.947 | −20.513 | 37.172 | 1.00 | 50.52 | A |
| ATOM | 2440 | CB | GLN | A | 362 | −40.441 | −20.727 | 37.082 | 1.00 | 46.02 | A |
| ATOM | 2441 | CG | GLN | A | 362 | −39.820 | −19.815 | 36.084 | 1.00 | 44.20 | A |
| ATOM | 2442 | CD | GLN | A | 362 | −38.320 | −19.912 | 36.032 | 1.00 | 45.88 | A |
| ATOM | 2443 | OE1 | GLN | A | 362 | −37.640 | −19.536 | 36.973 | 1.00 | 49.04 | A |
| ATOM | 2444 | NE2 | GLN | A | 362 | −37.789 | −20.409 | 34.922 | 1.00 | 45.80 | A |
| ATOM | 2445 | C | GLN | A | 362 | −42.637 | −21.673 | 37.892 | 1.00 | 52.25 | A |
| ATOM | 2446 | O | GLN | A | 362 | −43.084 | −22.628 | 37.274 | 1.00 | 52.41 | A |
| ATOM | 2447 | N | GLN | A | 363 | −42.754 | −21.566 | 39.207 | 1.00 | 55.22 | A |
| ATOM | 2448 | CA | GLN | A | 363 | −43.410 | −22.598 | 39.990 | 1.00 | 58.33 | A |
| ATOM | 2449 | CB | GLN | A | 363 | −43.057 | −22.437 | 41.472 | 1.00 | 61.87 | A |
| ATOM | 2450 | CG | GLN | A | 363 | −43.304 | −21.041 | 42.018 | 1.00 | 66.31 | A |
| ATOM | 2451 | CD | GLN | A | 363 | −42.782 | −20.848 | 43.433 | 1.00 | 68.07 | A |
| ATOM | 2452 | OE1 | GLN | A | 363 | −41.629 | −21.177 | 43.740 | 1.00 | 67.77 | A |
| ATOM | 2453 | NE2 | GLN | A | 363 | −43.628 | −20.294 | 44.299 | 1.00 | 69.15 | A |
| ATOM | 2454 | C | GLN | A | 363 | −44.921 | −22.527 | 39.794 | 1.00 | 58.98 | A |
| ATOM | 2455 | O | GLN | A | 363 | −45.691 | −23.038 | 40.608 | 1.00 | 60.45 | A |
| ATOM | 2456 | N | HIS | A | 364 | −45.344 | −21.890 | 38.709 | 1.00 | 58.27 | A |
| ATOM | 2457 | CA | HIS | A | 364 | −46.759 | −21.775 | 38.411 | 1.00 | 57.24 | A |
| ATOM | 2458 | CB | HIS | A | 364 | −47.276 | −20.390 | 38.785 | 1.00 | 60.54 | A |
| ATOM | 2459 | CG | HIS | A | 364 | −47.687 | −20.277 | 40.218 | 1.00 | 63.32 | A |
| ATOM | 2460 | CD2 | HIS | A | 364 | −47.093 | −19.673 | 41.273 | 1.00 | 63.98 | A |
| ATOM | 2461 | ND1 | HIS | A | 364 | −48.823 | −20.882 | 40.712 | 1.00 | 64.86 | A |
| ATOM | 2462 | CE1 | HIS | A | 364 | −48.910 | −20.656 | 42.011 | 1.00 | 65.50 | A |
| ATOM | 2463 | NE2 | HIS | A | 364 | −47.872 | −19.926 | 42.376 | 1.00 | 65.08 | A |
| ATOM | 2464 | C | HIS | A | 364 | −47.013 | −22.060 | 36.957 | 1.00 | 55.27 | A |
| ATOM | 2465 | O | HIS | A | 364 | −48.112 | −21.845 | 36.449 | 1.00 | 55.07 | A |
| ATOM | 2466 | N | GLY | A | 365 | −45.977 | −22.547 | 36.289 | 1.00 | 53.58 | A |
| ATOM | 2467 | CA | GLY | A | 365 | −46.101 | −22.891 | 34.887 | 1.00 | 53.30 | A |
| ATOM | 2468 | C | GLY | A | 365 | −45.703 | −21.831 | 33.881 | 1.00 | 52.25 | A |
| ATOM | 2469 | O | GLY | A | 365 | −45.615 | −22.126 | 32.693 | 1.00 | 54.12 | A |
| ATOM | 2470 | N | LEU | A | 366 | −45.468 | −20.605 | 34.331 | 1.00 | 49.61 | A |
| ATOM | 2471 | CA | LEU | A | 366 | −45.082 | −19.562 | 33.407 | 1.00 | 47.29 | A |
| ATOM | 2472 | CB | LEU | A | 366 | −45.458 | −18.204 | 33.984 | 1.00 | 47.17 | A |
| ATOM | 2473 | CG | LEU | A | 366 | −46.971 | −17.981 | 34.031 | 1.00 | 47.43 | A |
| ATOM | 2474 | CD1 | LEU | A | 366 | −47.280 | −16.657 | 34.695 | 1.00 | 46.56 | A |
| ATOM | 2475 | CD2 | LEU | A | 366 | −47.538 | −18.022 | 32.614 | 1.00 | 45.35 | A |
| ATOM | 2476 | C | LEU | A | 366 | −43.585 | −19.665 | 33.134 | 1.00 | 46.97 | A |
| ATOM | 2477 | O | LEU | A | 366 | −42.768 | −19.473 | 34.031 | 1.00 | 46.42 | A |
| ATOM | 2478 | N | ALA | A | 367 | −43.225 | −19.971 | 31.890 | 1.00 | 44.94 | A |
| ATOM | 2479 | CA | ALA | A | 367 | −41.824 | −20.129 | 31.541 | 1.00 | 44.43 | A |
| ATOM | 2480 | CB | ALA | A | 367 | −41.646 | −21.428 | 30.757 | 1.00 | 43.04 | A |
| ATOM | 2481 | C | ALA | A | 367 | −41.165 | −18.955 | 30.795 | 1.00 | 44.47 | A |
| ATOM | 2482 | O | ALA | A | 367 | −39.945 | −18.933 | 30.644 | 1.00 | 46.06 | A |
| ATOM | 2483 | N | THR | A | 368 | −41.955 | −17.984 | 30.338 | 1.00 | 43.20 | A |
| ATOM | 2484 | CA | THR | A | 368 | −41.426 | −16.813 | 29.630 | 1.00 | 41.33 | A |
| ATOM | 2485 | CB | THR | A | 368 | −42.453 | −16.270 | 28.617 | 1.00 | 41.77 | A |
| ATOM | 2486 | OG1 | THR | A | 368 | −43.561 | −15.655 | 29.300 | 1.00 | 40.83 | A |
| ATOM | 2487 | CG2 | THR | A | 368 | −42.969 | −17.396 | 27.768 | 1.00 | 41.99 | A |
| ATOM | 2488 | C | THR | A | 368 | −41.049 | −15.669 | 30.578 | 1.00 | 41.95 | A |
| ATOM | 2489 | O | THR | A | 368 | −41.343 | −15.711 | 31.776 | 1.00 | 43.10 | A |
| ATOM | 2490 | N | LEU | A | 369 | −40.405 | −14.636 | 30.039 | 1.00 | 42.08 | A |
| ATOM | 2491 | CA | LEU | A | 369 | −40.001 | −13.480 | 30.841 | 1.00 | 41.74 | A |
| ATOM | 2492 | CB | LEU | A | 369 | −38.754 | −12.828 | 30.257 | 1.00 | 39.32 | A |
| ATOM | 2493 | CG | LEU | A | 369 | −37.529 | −13.748 | 30.253 | 1.00 | 41.22 | A |
| ATOM | 2494 | CD1 | LEU | A | 369 | −36.377 | −13.100 | 29.480 | 1.00 | 40.10 | A |
| ATOM | 2495 | CD2 | LEU | A | 369 | −37.127 | −14.056 | 31.690 | 1.00 | 40.88 | A |
| ATOM | 2496 | C | LEU | A | 369 | −41.112 | −12.458 | 30.909 | 1.00 | 42.57 | A |
| ATOM | 2497 | O | LEU | A | 369 | −41.334 | −11.832 | 31.939 | 1.00 | 44.90 | A |
| ATOM | 2498 | N | GLY | A | 370 | −41.822 | −12.310 | 29.804 | 1.00 | 43.33 | A |
| ATOM | 2499 | CA | GLY | A | 370 | −42.915 | −11.359 | 29.730 | 1.00 | 44.61 | A |
| ATOM | 2500 | C | GLY | A | 370 | −43.259 | −11.102 | 28.273 | 1.00 | 45.37 | A |
| ATOM | 2501 | O | GLY | A | 370 | −42.466 | −11.419 | 27.369 | 1.00 | 45.99 | A |
| ATOM | 2502 | N | THR | A | 371 | −44.432 | −10.535 | 28.019 | 1.00 | 43.78 | A |
| ATOM | 2503 | CA | THR | A | 371 | −44.806 | −10.283 | 26.644 | 1.00 | 43.88 | A |
| ATOM | 2504 | CB | THR | A | 371 | −45.742 | −11.414 | 26.094 | 1.00 | 43.99 | A |
| ATOM | 2505 | OG1 | THR | A | 371 | −47.056 | −10.894 | 25.903 | 1.00 | 44.23 | A |
| ATOM | 2506 | CG2 | THR | A | 371 | −45.812 | −12.618 | 27.059 | 1.00 | 42.70 | A |
| ATOM | 2507 | C | THR | A | 371 | −45.449 | −8.917 | 26.427 | 1.00 | 44.02 | A |
| ATOM | 2508 | O | THR | A | 371 | −46.262 | −8.471 | 27.221 | 1.00 | 44.17 | A |
| ATOM | 2509 | N | LEU | A | 372 | −45.048 | −8.247 | 25.354 | 1.00 | 44.63 | A |
| ATOM | 2510 | CA | LEU | A | 372 | −45.594 | −6.947 | 25.005 | 1.00 | 46.38 | A |
| ATOM | 2511 | CB | LEU | A | 372 | −44.570 | −6.157 | 24.200 | 1.00 | 46.26 | A |
| ATOM | 2512 | CG | LEU | A | 372 | −43.308 | −5.709 | 24.932 | 1.00 | 46.99 | A |
| ATOM | 2513 | CD1 | LEU | A | 372 | −42.261 | −5.283 | 23.912 | 1.00 | 44.01 | A |
| ATOM | 2514 | CD2 | LEU | A | 372 | −43.644 | −4.579 | 25.905 | 1.00 | 44.60 | A |
| ATOM | 2515 | C | LEU | A | 372 | −46.873 | −7.086 | 24.165 | 1.00 | 48.29 | A |
| ATOM | 2516 | O | LEU | A | 372 | −47.024 | −8.020 | 23.380 | 1.00 | 48.64 | A |
| ATOM | 2517 | N | PHE | A | 373 | −47.790 | −6.144 | 24.339 | 1.00 | 50.33 | A |
| ATOM | 2518 | CA | PHE | A | 373 | −49.041 | −6.116 | 23.593 | 1.00 | 52.15 | A |
| ATOM | 2519 | CB | PHE | A | 373 | −50.143 | −5.571 | 24.492 | 1.00 | 51.89 | A |
| ATOM | 2520 | CG | PHE | A | 373 | −50.378 | −6.398 | 25.724 | 1.00 | 52.31 | A |
| ATOM | 2521 | CD1 | PHE | A | 373 | −51.164 | −5.913 | 26.765 | 1.00 | 53.13 | A |
| ATOM | 2522 | CD2 | PHE | A | 373 | −49.828 | −7.670 | 25.838 | 1.00 | 51.32 | A |
| ATOM | 2523 | CE1 | PHE | A | 373 | −51.398 | −6.686 | 27.899 | 1.00 | 52.70 | A |
| ATOM | 2524 | CE2 | PHE | A | 373 | −50.059 | −8.449 | 26.967 | 1.00 | 52.54 | A |
| ATOM | 2525 | CZ | PHE | A | 373 | −50.845 | −7.958 | 27.999 | 1.00 | 51.78 | A |
| ATOM | 2526 | C | PHE | A | 373 | −48.781 | −5.179 | 22.419 | 1.00 | 54.55 | A |
| ATOM | 2527 | O | PHE | A | 373 | −49.226 | −4.025 | 22.402 | 1.00 | 54.94 | A |
| ATOM | 2528 | N | SER | A | 374 | −48.040 | −5.700 | 21.446 | 1.00 | 57.26 | A |
| ATOM | 2529 | CA | SER | A | 374 | −47.610 | −4.965 | 20.260 | 1.00 | 58.86 | A |
| ATOM | 2530 | CB | SER | A | 374 | −46.807 | −5.905 | 19.375 | 1.00 | 59.85 | A |
| ATOM | 2531 | OG | SER | A | 374 | −45.847 | −6.593 | 20.163 | 1.00 | 63.14 | A |
| ATOM | 2532 | C | SER | A | 374 | −48.667 | −4.260 | 19.432 | 1.00 | 59.68 | A |
| ATOM | 2533 | O | SER | A | 374 | −48.615 | −3.048 | 19.273 | 1.00 | 59.04 | A |
| ATOM | 2534 | N | SER | A | 375 | −49.620 | −5.012 | 18.894 | 1.00 | 62.76 | A |
| ATOM | 2535 | CA | SER | A | 375 | −50.674 | −4.416 | 18.078 | 1.00 | 64.54 | A |
| ATOM | 2536 | CB | SER | A | 375 | −51.529 | −5.504 | 17.437 | 1.00 | 64.09 | A |
| ATOM | 2537 | OG | SER | A | 375 | −52.036 | −6.393 | 18.413 | 1.00 | 62.50 | A |
| ATOM | 2538 | C | SER | A | 375 | −51.554 | −3.479 | 18.897 | 1.00 | 66.62 | A |
| ATOM | 2539 | O | SER | A | 375 | −52.192 | −2.587 | 18.348 | 1.00 | 67.31 | A |
| ATOM | 2540 | N | MSE | A | 376 | −51.598 | −3.676 | 20.209 | 1.00 | 68.40 | A |
| ATOM | 2541 | CA | MSE | A | 376 | −52.393 | −2.798 | 21.051 | 1.00 | 70.23 | A |
| ATOM | 2542 | CB | MSE | A | 376 | −52.649 | −3.427 | 22.407 | 1.00 | 73.95 | A |
| ATOM | 2543 | CG | MSE | A | 376 | −53.917 | −4.236 | 22.441 | 1.00 | 79.99 | A |
| ATOM | 2544 | SE | MSE | A | 376 | −54.479 | −4.651 | 24.233 | 1.00 | 88.63 | A |
| ATOM | 2545 | CE | MSE | A | 376 | −54.369 | −6.590 | 24.104 | 1.00 | 85.55 | A |
| ATOM | 2546 | C | MSE | A | 376 | −51.702 | −1.465 | 21.243 | 1.00 | 70.45 | A |
| ATOM | 2547 | O | MSE | A | 376 | −52.362 | −.439 | 21.327 | 1.00 | 70.89 | A |
| ATOM | 2548 | N | MSE | A | 377 | −50.373 | −1.484 | 21.320 | 1.00 | 70.85 | A |
| ATOM | 2549 | CA | MSE | A | 377 | −49.591 | −.260 | 21.486 | 1.00 | 70.71 | A |
| ATOM | 2550 | CB | MSE | A | 377 | −48.184 | −.582 | 21.983 | 1.00 | 72.62 | A |
| ATOM | 2551 | CG | MSE | A | 377 | −48.096 | −1.171 | 23.376 | 1.00 | 75.08 | A |
| ATOM | 2552 | SE | MSE | A | 377 | −46.241 | −1.261 | 23.968 | 1.00 | 78.31 | A |
| ATOM | 2553 | CE | MSE | A | 377 | −46.007 | −3.176 | 23.851 | 1.00 | 76.05 | A |
| ATOM | 2554 | C | MSE | A | 377 | −49.468 | .469 | 20.154 | 1.00 | 70.00 | A |
| ATOM | 2555 | O | MSE | A | 377 | −49.510 | 1.695 | 20.093 | 1.00 | 69.65 | A |
| ATOM | 2556 | N | PHE | A | 378 | −49.297 | −.311 | 19.094 | 1.00 | 69.70 | A |
| ATOM | 2557 | CA | PHE | A | 378 | −49.149 | .203 | 17.735 | 1.00 | 69.58 | A |
| ATOM | 2558 | CB | PHE | A | 378 | −47.701 | .032 | 17.268 | 1.00 | 67.20 | A |
| ATOM | 2559 | CG | PHE | A | 378 | −46.689 | .516 | 18.253 | 1.00 | 65.72 | A |
| ATOM | 2560 | CD1 | PHE | A | 378 | −46.499 | 1.876 | 18.465 | 1.00 | 65.98 | A |
| ATOM | 2561 | CD2 | PHE | A | 378 | −45.937 | −.388 | 18.990 | 1.00 | 64.82 | A |
| ATOM | 2562 | CE1 | PHE | A | 378 | −45.573 | 2.331 | 19.400 | 1.00 | 66.24 | A |
| ATOM | 2563 | CE2 | PHE | A | 378 | −45.011 | .047 | 19.924 | 1.00 | 64.47 | A |
| ATOM | 2564 | CZ | PHE | A | 378 | −44.825 | 1.409 | 20.133 | 1.00 | 66.36 | A |
| ATOM | 2565 | C | PHE | A | 378 | −50.054 | −.623 | 16.827 | 1.00 | 70.40 | A |
| ATOM | 2566 | O | PHE | A | 378 | −49.591 | −1.574 | 16.192 | 1.00 | 70.39 | A |
| ATOM | 2567 | N | PRO | A | 379 | −51.355 | −.277 | 16.754 | 1.00 | 70.85 | A |
| ATOM | 2568 | CD | PRO | A | 379 | −52.044 | .793 | 17.494 | 1.00 | 70.34 | A |
| ATOM | 2569 | CA | PRO | A | 379 | −52.304 | −1.020 | 15.909 | 1.00 | 70.53 | A |
| ATOM | 2570 | CB | PRO | A | 379 | −53.643 | −.322 | 16.179 | 1.00 | 70.10 | A |
| ATOM | 2571 | CG | PRO | A | 379 | −53.242 | 1.049 | 16.614 | 1.00 | 70.94 | A |
| ATOM | 2572 | C | PRO | A | 379 | −51.964 | −1.115 | 14.423 | 1.00 | 69.96 | A |
| ATOM | 2573 | O | PRO | A | 379 | −52.455 | −2.006 | 13.727 | 1.00 | 69.06 | A |
| ATOM | 2574 | N | ASP | A | 380 | −51.112 | −.218 | 13.939 | 1.00 | 69.91 | A |
| ATOM | 2575 | CA | ASP | A | 380 | −50.722 | −.254 | 12.532 | 1.00 | 70.44 | A |
| ATOM | 2576 | CB | ASP | A | 380 | −50.045 | 1.055 | 12.128 | 1.00 | 72.28 | A |
| ATOM | 2577 | CG | ASP | A | 380 | −48.865 | 1.399 | 13.019 | 1.00 | 74.73 | A |
| ATOM | 2578 | OD1 | ASP | A | 380 | −48.056 | 2.270 | 12.622 | 1.00 | 75.17 | A |
| ATOM | 2579 | OD2 | ASP | A | 380 | −48.757 | .802 | 14.117 | 1.00 | 76.37 | A |
| ATOM | 2580 | C | ASP | A | 380 | −49.771 | −1.420 | 12.245 | 1.00 | 69.55 | A |
| ATOM | 2581 | O | ASP | A | 380 | −49.614 | −1.840 | 11.093 | 1.00 | 70.33 | A |
| ATOM | 2582 | N | ARG | A | 381 | −49.138 | −1.944 | 13.289 | 1.00 | 66.83 | A |
| ATOM | 2583 | CA | ARG | A | 381 | −48.207 | −3.050 | 13.119 | 1.00 | 64.70 | A |
| ATOM | 2584 | CB | ARG | A | 381 | −47.419 | −3.283 | 14.409 | 1.00 | 63.09 | A |
| ATOM | 2585 | CG | ARG | A | 381 | −46.721 | −2.055 | 14.973 | 1.00 | 60.19 | A |
| ATOM | 2586 | CD | ARG | A | 381 | −45.448 | −1.739 | 14.232 | 1.00 | 56.81 | A |
| ATOM | 2587 | NE | ARG | A | 381 | −44.671 | −.692 | 14.895 | 1.00 | 56.00 | A |
| ATOM | 2588 | CZ | ARG | A | 381 | −44.028 | −.842 | 16.054 | 1.00 | 54.80 | A |
| ATOM | 2589 | NH1 | ARG | A | 381 | −44.064 | −2.003 | 16.693 | 1.00 | 52.20 | A |
| ATOM | 2590 | NH2 | ARG | A | 381 | −43.338 | .173 | 16.570 | 1.00 | 54.33 | A |
| ATOM | 2591 | C | ARG | A | 381 | −48.889 | −4.359 | 12.711 | 1.00 | 65.08 | A |
| ATOM | 2592 | O | ARG | A | 381 | −48.201 | −5.362 | 12.485 | 1.00 | 64.38 | A |
| ATOM | 2593 | N | ALA | A | 382 | −50.223 | −4.368 | 12.613 | 1.00 | 65.21 | A |
| ATOM | 2594 | CA | ALA | A | 382 | −50.928 | −5.603 | 12.242 | 1.00 | 66.00 | A |
| ATOM | 2595 | CB | ALA | A | 382 | −50.747 | −6.649 | 13.349 | 1.00 | 64.03 | A |
| ATOM | 2596 | C | ALA | A | 382 | −52.417 | −5.466 | 11.912 | 1.00 | 66.34 | A |
| ATOM | 2597 | O | ALA | A | 382 | −53.050 | −4.474 | 12.267 | 1.00 | 66.09 | A |
| ATOM | 2598 | N | PRO | A | 383 | −52.988 | −6.474 | 11.214 | 1.00 | 67.29 | A |
| ATOM | 2599 | CD | PRO | A | 383 | −52.279 | −7.632 | 10.637 | 1.00 | 67.33 | A |
| ATOM | 2600 | CA | PRO | A | 383 | −54.402 | −6.508 | 10.823 | 1.00 | 68.26 | A |
| ATOM | 2601 | CB | PRO | A | 383 | −54.545 | −7.881 | 10.178 | 1.00 | 67.13 | A |
| ATOM | 2602 | CG | PRO | A | 383 | −53.225 | −8.087 | 9.557 | 1.00 | 66.90 | A |
| ATOM | 2603 | C | PRO | A | 383 | −55.314 | −6.357 | 12.040 | 1.00 | 69.16 | A |
| ATOM | 2604 | O | PRO | A | 383 | −55.137 | −7.053 | 13.046 | 1.00 | 69.21 | A |
| ATOM | 2605 | N | ASN | A | 384 | −56.293 | −5.461 | 11.934 | 1.00 | 69.58 | A |
| ATOM | 2606 | CA | ASN | A | 384 | −57.234 | −5.189 | 13.024 | 1.00 | 69.59 | A |
| ATOM | 2607 | CB | ASN | A | 384 | −58.178 | −4.056 | 12.617 | 1.00 | 70.28 | A |
| ATOM | 2608 | CG | ASN | A | 384 | −58.885 | −4.338 | 11.308 | 1.00 | 71.93 | A |
| ATOM | 2609 | OD1 | ASN | A | 384 | −58.245 | −4.489 | 10.260 | 1.00 | 71.96 | A |
| ATOM | 2610 | ND2 | ASN | A | 384 | −60.212 | −4.413 | 11.357 | 1.00 | 72.09 | A |
| ATOM | 2611 | C | ASN | A | 384 | −58.064 | −6.372 | 13.538 | 1.00 | 68.05 | A |
| ATOM | 2612 | O | ASN | A | 384 | −58.840 | −6.208 | 14.468 | 1.00 | 68.07 | A |
| ATOM | 2613 | N | ASN | A | 385 | −57.912 | −7.552 | 12.951 | 1.00 | 66.86 | A |
| ATOM | 2614 | CA | ASN | A | 385 | −58.674 | −8.714 | 13.406 | 1.00 | 66.85 | A |
| ATOM | 2615 | CB | ASN | A | 385 | −59.381 | −9.384 | 12.227 | 1.00 | 68.55 | A |
| ATOM | 2616 | CG | ASN | A | 385 | −58.405 | −10.010 | 11.238 | 1.00 | 69.12 | A |
| ATOM | 2617 | OD1 | ASN | A | 385 | −57.611 | −9.309 | 10.604 | 1.00 | 70.33 | A |
| ATOM | 2618 | ND2 | ASN | A | 385 | −58.459 | −11.334 | 11.106 | 1.00 | 68.91 | A |
| ATOM | 2619 | C | ASN | A | 385 | −57.733 | −9.720 | 14.054 | 1.00 | 66.28 | A |
| ATOM | 2620 | O | ASN | A | 385 | −58.043 | −10.918 | 14.163 | 1.00 | 65.36 | A |
| ATOM | 2621 | N | VAL | A | 386 | −56.580 | −9.221 | 14.493 | 1.00 | 65.56 | A |
| ATOM | 2622 | CA | VAL | A | 386 | −55.563 | −10.074 | 15.109 | 1.00 | 63.10 | A |
| ATOM | 2623 | CB | VAL | A | 386 | −54.611 | −10.627 | 13.984 | 1.00 | 61.21 | A |
| ATOM | 2624 | CG1 | VAL | A | 386 | −53.686 | −9.538 | 13.500 | 1.00 | 59.46 | A |
| ATOM | 2625 | CG2 | VAL | A | 386 | −53.855 | −11.831 | 14.467 | 1.00 | 60.91 | A |
| ATOM | 2626 | C | VAL | A | 386 | −54.771 | −9.310 | 16.195 | 1.00 | 60.17 | A |
| ATOM | 2627 | O | VAL | A | 386 | −54.816 | −8.081 | 16.258 | 1.00 | 59.27 | A |
| ATOM | 2628 | N | TYR | A | 387 | −54.079 | −10.055 | 17.056 | 1.00 | 57.52 | A |
| ATOM | 2629 | CA | TYR | A | 387 | −53.261 | −9.491 | 18.136 | 1.00 | 54.09 | A |
| ATOM | 2630 | CB | TYR | A | 387 | −53.658 | −10.089 | 19.482 | 1.00 | 54.61 | A |
| ATOM | 2631 | CG | TYR | A | 387 | −54.961 | −9.593 | 20.051 | 1.00 | 53.78 | A |
| ATOM | 2632 | CD1 | TYR | A | 387 | −55.854 | −10.473 | 20.664 | 1.00 | 51.70 | A |
| ATOM | 2633 | CE1 | TYR | A | 387 | −57.037 | −10.021 | 21.226 | 1.00 | 51.99 | A |
| ATOM | 2634 | CD2 | TYR | A | 387 | −55.285 | −8.243 | 20.016 | 1.00 | 54.25 | A |
| ATOM | 2635 | CE2 | TYR | A | 387 | −56.466 | −7.775 | 20.581 | 1.00 | 54.78 | A |
| ATOM | 2636 | CZ | TYR | A | 387 | −57.339 | −8.670 | 21.184 | 1.00 | 53.57 | A |
| ATOM | 2637 | OH | TYR | A | 387 | −58.507 | −8.202 | 21.753 | 1.00 | 55.41 | A |
| ATOM | 2638 | C | TYR | A | 387 | −51.808 | −9.858 | 17.899 | 1.00 | 52.38 | A |
| ATOM | 2639 | O | TYR | A | 387 | −51.490 | −11.033 | 17.696 | 1.00 | 50.35 | A |
| ATOM | 2640 | N | LEU | A | 388 | −50.927 | −8.865 | 17.928 | 1.00 | 50.82 | A |
| ATOM | 2641 | CA | LEU | A | 388 | −49.503 | −9.128 | 17.733 | 1.00 | 50.79 | A |
| ATOM | 2642 | CB | LEU | A | 388 | −48.882 | −8.083 | 16.802 | 1.00 | 48.14 | A |
| ATOM | 2643 | CG | LEU | A | 388 | −47.410 | −8.380 | 16.500 | 1.00 | 48.54 | A |
| ATOM | 2644 | CD1 | LEU | A | 388 | −47.276 | −9.799 | 15.964 | 1.00 | 47.79 | A |
| ATOM | 2645 | CD2 | LEU | A | 388 | −46.855 | −7.368 | 15.524 | 1.00 | 47.68 | A |
| ATOM | 2646 | C | LEU | A | 388 | −48.802 | −9.102 | 19.098 | 1.00 | 51.74 | A |
| ATOM | 2647 | O | LEU | A | 388 | −48.758 | −8.056 | 19.762 | 1.00 | 51.70 | A |
| ATOM | 2648 | N | TYR | A | 389 | −48.249 | −10.247 | 19.505 | 1.00 | 50.42 | A |
| ATOM | 2649 | CA | TYR | A | 389 | −47.588 | −10.369 | 20.807 | 1.00 | 49.75 | A |
| ATOM | 2650 | CB | TYR | A | 389 | −48.215 | −11.527 | 21.594 | 1.00 | 48.91 | A |
| ATOM | 2651 | CG | TYR | A | 389 | −49.660 | −11.341 | 21.967 | 1.00 | 47.97 | A |
| ATOM | 2652 | CD1 | TYR | A | 389 | −50.533 | −12.420 | 21.982 | 1.00 | 48.36 | A |
| ATOM | 2653 | CE1 | TYR | A | 389 | −51.878 | −12.259 | 22.329 | 1.00 | 48.24 | A |
| ATOM | 2654 | CD2 | TYR | A | 389 | −50.157 | −10.089 | 22.314 | 1.00 | 49.07 | A |
| ATOM | 2655 | CE2 | TYR | A | 389 | −51.493 | −9.916 | 22.667 | 1.00 | 49.60 | A |
| ATOM | 2656 | CZ | TYR | A | 389 | −52.348 | −11.011 | 22.669 | 1.00 | 48.87 | A |
| ATOM | 2657 | OH | TYR | A | 389 | −53.669 | −10.855 | 23.009 | 1.00 | 47.68 | A |
| ATOM | 2658 | C | TYR | A | 389 | −46.084 | −10.606 | 20.770 | 1.00 | 49.84 | A |
| ATOM | 2659 | O | TYR | A | 389 | −45.637 | −11.673 | 20.337 | 1.00 | 52.88 | A |
| ATOM | 2660 | N | THR | A | 390 | −45.294 | −9.647 | 21.241 | 1.00 | 47.84 | A |
| ATOM | 2661 | CA | THR | A | 390 | −43.845 | −9.860 | 21.275 | 1.00 | 46.50 | A |
| ATOM | 2662 | CB | THR | A | 390 | −43.048 | −8.557 | 21.070 | 1.00 | 45.30 | A |
| ATOM | 2663 | OG1 | THR | A | 390 | −43.222 | −8.079 | 19.737 | 1.00 | 44.09 | A |
| ATOM | 2664 | CG2 | THR | A | 390 | −41.579 | −8.810 | 21.270 | 1.00 | 46.34 | A |
| ATOM | 2665 | C | THR | A | 390 | −43.461 | −10.460 | 22.635 | 1.00 | 46.76 | A |
| ATOM | 2666 | O | THR | A | 390 | −43.567 | −9.789 | 23.665 | 1.00 | 46.08 | A |
| ATOM | 2667 | N | THR | A | 391 | −43.014 | −11.717 | 22.625 | 1.00 | 45.86 | A |
| ATOM | 2668 | CA | THR | A | 391 | −42.626 | −12.428 | 23.835 | 1.00 | 44.75 | A |
| ATOM | 2669 | CB | THR | A | 391 | −43.309 | −13.782 | 23.895 | 1.00 | 45.35 | A |
| ATOM | 2670 | OG1 | THR | A | 391 | −44.723 | −13.584 | 23.876 | 1.00 | 48.01 | A |
| ATOM | 2671 | CG2 | THR | A | 391 | −42.909 | −14.539 | 25.159 | 1.00 | 46.29 | A |
| ATOM | 2672 | C | THR | A | 391 | −41.129 | −12.666 | 23.951 | 1.00 | 45.58 | A |
| ATOM | 2673 | O | THR | A | 391 | −40.460 | −12.991 | 22.970 | 1.00 | 43.84 | A |
| ATOM | 2674 | N | PHE | A | 392 | −40.614 | −12.515 | 25.172 | 1.00 | 46.94 | A |
| ATOM | 2675 | CA | PHE | A | 392 | −39.194 | −12.729 | 25.453 | 1.00 | 45.90 | A |
| ATOM | 2676 | CB | PHE | A | 392 | −38.640 | −11.545 | 26.227 | 1.00 | 42.09 | A |
| ATOM | 2677 | CG | PHE | A | 392 | −38.819 | −10.248 | 25.514 | 1.00 | 43.60 | A |
| ATOM | 2678 | CD1 | PHE | A | 392 | −39.934 | −9.453 | 25.761 | 1.00 | 44.61 | A |
| ATOM | 2679 | CD2 | PHE | A | 392 | −37.904 | −9.835 | 24.550 | 1.00 | 43.13 | A |
| ATOM | 2680 | CE1 | PHE | A | 392 | −40.138 | −8.269 | 25.063 | 1.00 | 42.85 | A |
| ATOM | 2681 | CE2 | PHE | A | 392 | −38.099 | −8.655 | 23.848 | 1.00 | 42.37 | A |
| ATOM | 2682 | CZ | PHE | A | 392 | −39.221 | −7.873 | 24.107 | 1.00 | 43.34 | A |
| ATOM | 2683 | C | PHE | A | 392 | −38.991 | −14.024 | 26.230 | 1.00 | 46.12 | A |
| ATOM | 2684 | O | PHE | A | 392 | −39.594 | −14.224 | 27.275 | 1.00 | 46.62 | A |
| ATOM | 2685 | N | VAL | A | 393 | −38.150 | −14.907 | 25.703 | 1.00 | 46.83 | A |
| ATOM | 2686 | CA | VAL | A | 393 | −37.897 | −16.187 | 26.349 | 1.00 | 48.34 | A |
| ATOM | 2687 | CB | VAL | A | 393 | −38.260 | −17.346 | 25.415 | 1.00 | 47.89 | A |
| ATOM | 2688 | CG1 | VAL | A | 393 | −38.163 | −18.665 | 26.157 | 1.00 | 48.15 | A |
| ATOM | 2689 | CG2 | VAL | A | 393 | −39.657 | −17.147 | 24.871 | 1.00 | 50.59 | A |
| ATOM | 2690 | C | VAL | A | 393 | −36.441 | −16.360 | 26.762 | 1.00 | 49.26 | A |
| ATOM | 2691 | O | VAL | A | 393 | −35.545 | −15.746 | 26.180 | 1.00 | 50.23 | A |
| ATOM | 2692 | N | GLY | A | 394 | −36.209 | −17.195 | 27.769 | 1.00 | 48.91 | A |
| ATOM | 2693 | CA | GLY | A | 394 | −34.847 | −17.440 | 28.208 | 1.00 | 49.61 | A |
| ATOM | 2694 | C | GLY | A | 394 | −34.437 | −16.611 | 29.401 | 1.00 | 49.58 | A |
| ATOM | 2695 | O | GLY | A | 394 | −35.063 | −16.682 | 30.448 | 1.00 | 51.10 | A |
| ATOM | 2696 | N | GLY | A | 395 | −33.382 | −15.825 | 29.253 | 1.00 | 48.68 | A |
| ATOM | 2697 | CA | GLY | A | 395 | −32.950 | −14.998 | 30.359 | 1.00 | 48.14 | A |
| ATOM | 2698 | C | GLY | A | 395 | −32.186 | −15.776 | 31.405 | 1.00 | 47.50 | A |
| ATOM | 2699 | O | GLY | A | 395 | −32.077 | −16.984 | 31.331 | 1.00 | 47.39 | A |
| ATOM | 2700 | N | SER | A | 396 | −31.664 | −15.070 | 32.397 | 1.00 | 49.41 | A |
| ATOM | 2701 | CA | SER | A | 396 | −30.893 | −15.694 | 33.458 | 1.00 | 50.98 | A |
| ATOM | 2702 | CB | SER | A | 396 | −30.491 | −14.654 | 34.506 | 1.00 | 50.57 | A |
| ATOM | 2703 | OG | SER | A | 396 | −29.606 | −13.695 | 33.957 | 1.00 | 50.88 | A |
| ATOM | 2704 | C | SER | A | 396 | −31.612 | −16.836 | 34.145 | 1.00 | 51.21 | A |
| ATOM | 2705 | O | SER | A | 396 | −30.979 | −17.806 | 34.528 | 1.00 | 53.42 | A |
| ATOM | 2706 | N | ARG | A | 397 | −32.927 | −16.733 | 34.294 | 1.00 | 51.37 | A |
| ATOM | 2707 | CA | ARG | A | 397 | −33.687 | −17.780 | 34.963 | 1.00 | 51.64 | A |
| ATOM | 2708 | CB | ARG | A | 397 | −35.129 | −17.324 | 35.210 | 1.00 | 50.23 | A |
| ATOM | 2709 | CG | ARG | A | 397 | −36.028 | −17.334 | 33.988 | 1.00 | 49.28 | A |
| ATOM | 2710 | CD | ARG | A | 397 | −37.384 | −16.681 | 34.274 | 1.00 | 48.98 | A |
| ATOM | 2711 | NE | ARG | A | 397 | −37.248 | −15.261 | 34.614 | 1.00 | 48.94 | A |
| ATOM | 2712 | CZ | ARG | A | 397 | −38.274 | −14.462 | 34.885 | 1.00 | 46.85 | A |
| ATOM | 2713 | NH1 | ARG | A | 397 | −39.499 | −14.953 | 34.850 | 1.00 | 46.93 | A |
| ATOM | 2714 | NH2 | ARG | A | 397 | −38.082 | −13.186 | 35.196 | 1.00 | 44.39 | A |
| ATOM | 2715 | C | ARG | A | 397 | −33.706 | −19.084 | 34.185 | 1.00 | 53.60 | A |
| ATOM | 2716 | O | ARG | A | 397 | −34.041 | −20.135 | 34.731 | 1.00 | 54.77 | A |
| ATOM | 2717 | N | ASN | A | 398 | −33.334 | −19.024 | 32.914 | 1.00 | 54.39 | A |
| ATOM | 2718 | CA | ASN | A | 398 | −33.372 | −20.212 | 32.094 | 1.00 | 55.65 | A |
| ATOM | 2719 | CB | ASN | A | 398 | −34.830 | −20.608 | 31.875 | 1.00 | 55.66 | A |
| ATOM | 2720 | CG | ASN | A | 398 | −34.977 | −21.990 | 31.304 | 1.00 | 55.52 | A |
| ATOM | 2721 | OD1 | ASN | A | 398 | −34.083 | −22.824 | 31.425 | 1.00 | 54.90 | A |
| ATOM | 2722 | ND2 | ASN | A | 398 | −36.123 | −22.252 | 30.697 | 1.00 | 56.78 | A |
| ATOM | 2723 | C | ASN | A | 398 | −32.690 | −19.983 | 30.760 | 1.00 | 57.04 | A |
| ATOM | 2724 | O | ASN | A | 398 | −33.340 | −19.642 | 29.768 | 1.00 | 57.38 | A |
| ATOM | 2725 | N | ARG | A | 399 | −31.379 | −20.194 | 30.746 | 1.00 | 57.10 | A |
| ATOM | 2726 | CA | ARG | A | 399 | −30.571 | −20.009 | 29.554 | 1.00 | 57.82 | A |
| ATOM | 2727 | CB | ARG | A | 399 | −29.105 | −20.021 | 29.945 | 1.00 | 58.10 | A |
| ATOM | 2728 | CG | ARG | A | 399 | −28.712 | −18.844 | 30.814 | 1.00 | 57.78 | A |
| ATOM | 2729 | CD | ARG | A | 399 | −27.358 | −19.049 | 31.471 | 1.00 | 58.77 | A |
| ATOM | 2730 | NE | ARG | A | 399 | −26.883 | −17.815 | 32.079 | 1.00 | 60.50 | A |
| ATOM | 2731 | CZ | ARG | A | 399 | −26.161 | −16.895 | 31.444 | 1.00 | 63.05 | A |
| ATOM | 2732 | NH1 | ARG | A | 399 | −25.813 | −17.069 | 30.174 | 1.00 | 64.49 | A |
| ATOM | 2733 | NH2 | ARG | A | 399 | −25.806 | −15.781 | 32.070 | 1.00 | 64.35 | A |
| ATOM | 2734 | C | ARG | A | 399 | −30.818 | −21.015 | 28.439 | 1.00 | 59.24 | A |
| ATOM | 2735 | O | ARG | A | 399 | −30.893 | −20.640 | 27.272 | 1.00 | 59.56 | A |
| ATOM | 2736 | N | GLU | A | 400 | −30.940 | −22.291 | 28.781 | 1.00 | 61.56 | A |
| ATOM | 2737 | CA | GLU | A | 400 | −31.182 | −23.309 | 27.760 | 1.00 | 64.68 | A |
| ATOM | 2738 | CB | GLU | A | 400 | −30.907 | −24.718 | 28.311 | 1.00 | 67.84 | A |
| ATOM | 2739 | CG | GLU | A | 400 | −29.430 | −25.071 | 28.448 | 1.00 | 73.15 | A |
| ATOM | 2740 | CD | GLU | A | 400 | −28.629 | −24.758 | 27.187 | 1.00 | 76.21 | A |
| ATOM | 2741 | OE1 | GLU | A | 400 | −29.081 | −25.134 | 26.079 | 1.00 | 77.10 | A |
| ATOM | 2742 | OE2 | GLU | A | 400 | −27.544 | −24.138 | 27.308 | 1.00 | 77.97 | A |
| ATOM | 2743 | C | GLU | A | 400 | −32.600 | −23.270 | 27.186 | 1.00 | 64.54 | A |
| ATOM | 2744 | O | GLU | A | 400 | −33.073 | −24.255 | 26.631 | 1.00 | 65.76 | A |
| ATOM | 2745 | N | LEU | A | 401 | −33.285 | −22.144 | 27.333 | 1.00 | 63.62 | A |
| ATOM | 2746 | CA | LEU | A | 401 | −34.631 | −22.019 | 26.805 | 1.00 | 61.99 | A |
| ATOM | 2747 | CB | LEU | A | 401 | −35.576 | −21.530 | 27.890 | 1.00 | 60.59 | A |
| ATOM | 2748 | CG | LEU | A | 401 | −37.040 | −21.626 | 27.473 | 1.00 | 60.40 | A |
| ATOM | 2749 | CD1 | LEU | A | 401 | −37.361 | −23.090 | 27.252 | 1.00 | 60.63 | A |
| ATOM | 2750 | CD2 | LEU | A | 401 | −37.957 | −21.028 | 28.528 | 1.00 | 58.43 | A |
| ATOM | 2751 | C | LEU | A | 401 | −34.588 | −21.008 | 25.668 | 1.00 | 62.87 | A |
| ATOM | 2752 | O | LEU | A | 401 | −35.465 | −20.972 | 24.796 | 1.00 | 62.93 | A |
| ATOM | 2753 | N | ALA | A | 402 | −33.543 | −20.188 | 25.689 | 1.00 | 62.25 | A |
| ATOM | 2754 | CA | ALA | A | 402 | −33.342 | −19.164 | 24.687 | 1.00 | 61.40 | A |
| ATOM | 2755 | CB | ALA | A | 402 | −32.304 | −18.175 | 25.178 | 1.00 | 60.70 | A |
| ATOM | 2756 | C | ALA | A | 402 | −32.890 | −19.802 | 23.384 | 1.00 | 61.74 | A |
| ATOM | 2757 | O | ALA | A | 402 | −33.147 | −19.276 | 22.303 | 1.00 | 61.40 | A |
| ATOM | 2758 | N | LYS | A | 403 | −32.226 | −20.948 | 23.497 | 1.00 | 63.19 | A |
| ATOM | 2759 | CA | LYS | A | 403 | −31.720 | −21.668 | 22.330 | 1.00 | 65.11 | A |
| ATOM | 2760 | CB | LYS | A | 403 | −30.345 | −22.260 | 22.641 | 1.00 | 65.02 | A |
| ATOM | 2761 | CG | LYS | A | 403 | −29.389 | −21.266 | 23.256 | 1.00 | 67.24 | A |
| ATOM | 2762 | CD | LYS | A | 403 | −28.003 | −21.842 | 23.380 | 1.00 | 69.45 | A |
| ATOM | 2763 | CE | LYS | A | 403 | −28.014 | −23.129 | 24.184 | 1.00 | 71.23 | A |
| ATOM | 2764 | NZ | LYS | A | 403 | −26.629 | −23.660 | 24.354 | 1.00 | 73.08 | A |
| ATOM | 2765 | C | LYS | A | 403 | −32.638 | −22.780 | 21.805 | 1.00 | 66.00 | A |
| ATOM | 2766 | O | LYS | A | 403 | −32.203 | −23.621 | 21.018 | 1.00 | 67.20 | A |
| ATOM | 2767 | N | ALA | A | 404 | −33.898 | −22.781 | 22.231 | 1.00 | 66.38 | A |
| ATOM | 2768 | CA | ALA | A | 404 | −34.854 | −23.788 | 21.784 | 1.00 | 66.59 | A |
| ATOM | 2769 | CB | ALA | A | 404 | −36.130 | −23.709 | 22.616 | 1.00 | 67.25 | A |
| ATOM | 2770 | C | ALA | A | 404 | −35.195 | −23.609 | 20.312 | 1.00 | 66.67 | A |
| ATOM | 2771 | O | ALA | A | 404 | −35.014 | −22.524 | 19.750 | 1.00 | 66.46 | A |
| ATOM | 2772 | N | SER | A | 405 | −35.682 | −24.687 | 19.698 | 1.00 | 67.34 | A |
| ATOM | 2773 | CA | SER | A | 405 | −36.083 | −24.678 | 18.290 | 1.00 | 67.23 | A |
| ATOM | 2774 | CB | SER | A | 405 | −36.415 | −26.099 | 17.833 | 1.00 | 66.96 | A |
| ATOM | 2775 | OG | SER | A | 405 | −37.519 | −26.632 | 18.555 | 1.00 | 65.54 | A |
| ATOM | 2776 | C | SER | A | 405 | −37.324 | −23.796 | 18.155 | 1.00 | 67.86 | A |
| ATOM | 2777 | O | SER | A | 405 | −38.088 | −23.652 | 19.119 | 1.00 | 68.30 | A |
| ATOM | 2778 | N | ARG | A | 406 | −37.538 | −23.208 | 16.978 | 1.00 | 67.80 | A |
| ATOM | 2779 | CA | ARG | A | 406 | −38.709 | −22.351 | 16.811 | 1.00 | 67.68 | A |
| ATOM | 2780 | CB | ARG | A | 406 | −38.851 | −21.852 | 15.367 | 1.00 | 67.93 | A |
| ATOM | 2781 | CG | ARG | A | 406 | −40.095 | −20.979 | 15.160 | 1.00 | 69.27 | A |
| ATOM | 2782 | CD | ARG | A | 406 | −40.076 | −20.222 | 13.836 | 1.00 | 71.62 | A |
| ATOM | 2783 | NE | ARG | A | 406 | −38.967 | −19.270 | 13.769 | 1.00 | 74.16 | A |
| ATOM | 2784 | CZ | ARG | A | 406 | −38.705 | −18.486 | 12.724 | 1.00 | 74.37 | A |
| ATOM | 2785 | NH1 | ARG | A | 406 | −39.478 | −18.531 | 11.646 | 1.00 | 72.40 | A |
| ATOM | 2786 | NH2 | ARG | A | 406 | −37.658 | −17.662 | 12.755 | 1.00 | 75.07 | A |
| ATOM | 2787 | C | ARG | A | 406 | −39.970 | −23.101 | 17.234 | 1.00 | 66.40 | A |
| ATOM | 2788 | O | ARG | A | 406 | −40.915 | −22.501 | 17.752 | 1.00 | 65.35 | A |
| ATOM | 2789 | N | THR | A | 407 | −39.981 | −24.413 | 17.029 | 1.00 | 65.49 | A |
| ATOM | 2790 | CA | THR | A | 407 | −41.139 | −25.189 | 17.425 | 1.00 | 66.10 | A |
| ATOM | 2791 | CB | THR | A | 407 | −41.026 | −26.674 | 17.017 | 1.00 | 66.06 | A |
| ATOM | 2792 | OG1 | THR | A | 407 | −40.951 | −26.771 | 15.590 | 1.00 | 65.68 | A |
| ATOM | 2793 | CG2 | THR | A | 407 | −42.250 | −27.452 | 17.495 | 1.00 | 64.80 | A |
| ATOM | 2794 | C | THR | A | 407 | −41.259 | −25.084 | 18.935 | 1.00 | 66.86 | A |
| ATOM | 2795 | O | THR | A | 407 | −42.214 | −24.475 | 19.429 | 1.00 | 68.25 | A |
| ATOM | 2796 | N | GLU | A | 408 | −40.290 | −25.648 | 19.663 | 1.00 | 66.00 | A |
| ATOM | 2797 | CA | GLU | A | 408 | −40.302 | −25.602 | 21.132 | 1.00 | 65.88 | A |
| ATOM | 2798 | CB | GLU | A | 408 | −38.961 | −26.082 | 21.701 | 1.00 | 67.78 | A |
| ATOM | 2799 | CG | GLU | A | 408 | −38.909 | −27.593 | 21.914 | 1.00 | 71.24 | A |
| ATOM | 2800 | CD | GLU | A | 408 | −37.514 | −28.199 | 21.729 | 1.00 | 74.06 | A |
| ATOM | 2801 | OE1 | GLU | A | 408 | −37.385 | −29.426 | 21.949 | 1.00 | 74.79 | A |
| ATOM | 2802 | OE2 | GLU | A | 408 | −36.557 | −27.468 | 21.362 | 1.00 | 74.43 | A |
| ATOM | 2803 | C | GLU | A | 408 | −40.625 | −24.209 | 21.659 | 1.00 | 63.90 | A |
| ATOM | 2804 | O | GLU | A | 408 | −41.348 | −24.062 | 22.642 | 1.00 | 62.90 | A |
| ATOM | 2805 | N | LEU | A | 409 | −40.088 | −23.186 | 21.004 | 1.00 | 62.20 | A |
| ATOM | 2806 | CA | LEU | A | 409 | −40.369 | −21.818 | 21.408 | 1.00 | 60.44 | A |
| ATOM | 2807 | CB | LEU | A | 409 | −39.519 | −20.824 | 20.604 | 1.00 | 59.56 | A |
| ATOM | 2808 | CG | LEU | A | 409 | −38.028 | −20.781 | 20.964 | 1.00 | 58.85 | A |
| ATOM | 2809 | CD1 | LEU | A | 409 | −37.275 | −19.911 | 19.985 | 1.00 | 55.21 | A |
| ATOM | 2810 | CD2 | LEU | A | 409 | −37.861 | −20.262 | 22.390 | 1.00 | 57.51 | A |
| ATOM | 2811 | C | LEU | A | 409 | −41.844 | −21.552 | 21.171 | 1.00 | 59.10 | A |
| ATOM | 2812 | O | LEU | A | 409 | −42.523 | −21.027 | 22.040 | 1.00 | 59.87 | A |
| ATOM | 2813 | N | LYS | A | 410 | −42.339 | −21.934 | 19.998 | 1.00 | 58.11 | A |
| ATOM | 2814 | CA | LYS | A | 410 | −43.742 | −21.732 | 19.660 | 1.00 | 57.54 | A |
| ATOM | 2815 | CB | LYS | A | 410 | −44.077 | −22.434 | 18.342 | 1.00 | 58.16 | A |
| ATOM | 2816 | CG | LYS | A | 410 | −45.454 | −22.085 | 17.808 | 1.00 | 61.02 | A |
| ATOM | 2817 | CD | LYS | A | 410 | −45.926 | −23.046 | 16.724 | 1.00 | 64.12 | A |
| ATOM | 2818 | CE | LYS | A | 410 | −47.368 | −22.720 | 16.305 | 1.00 | 67.43 | A |
| ATOM | 2819 | NZ | LYS | A | 410 | −47.939 | −23.574 | 15.209 | 1.00 | 68.06 | A |
| ATOM | 2820 | C | LYS | A | 410 | −44.667 | −22.247 | 20.777 | 1.00 | 56.58 | A |
| ATOM | 2821 | O | LYS | A | 410 | −45.604 | −21.562 | 21.189 | 1.00 | 54.99 | A |
| ATOM | 2822 | N | GLU | A | 411 | −44.413 | −23.448 | 21.276 | 1.00 | 55.57 | A |
| ATOM | 2823 | CA | GLU | A | 411 | −45.256 | −23.966 | 22.339 | 1.00 | 57.01 | A |
| ATOM | 2824 | CB | GLU | A | 411 | −45.002 | −25.458 | 22.558 | 1.00 | 58.25 | A |
| ATOM | 2825 | CG | GLU | A | 411 | −45.729 | −26.356 | 21.554 | 1.00 | 59.41 | A |
| ATOM | 2826 | CD | GLU | A | 411 | −47.232 | −26.302 | 21.713 | .00 | 58.79 | A |
| ATOM | 2827 | OE1 | GLU | A | 411 | −47.725 | −26.616 | 22.817 | .00 | 58.86 | A |
| ATOM | 2828 | OE2 | GLU | A | 411 | −47.920 | −25.947 | 20.735 | .00 | 58.86 | A |
| ATOM | 2829 | C | GLU | A | 411 | −45.082 | −23.215 | 23.659 | 1.00 | 57.02 | A |
| ATOM | 2830 | O | GLU | A | 411 | −46.066 | −22.886 | 24.310 | 1.00 | 58.00 | A |
| ATOM | 2831 | N | ILE | A | 412 | −43.838 | −22.947 | 24.056 | 1.00 | 56.82 | A |
| ATOM | 2832 | CA | ILE | A | 412 | −43.579 | −22.232 | 25.307 | 1.00 | 55.05 | A |
| ATOM | 2833 | CB | ILE | A | 412 | −42.087 | −21.822 | 25.457 | 1.00 | 54.61 | A |
| ATOM | 2834 | CG2 | ILE | A | 412 | −41.888 | −21.066 | 26.765 | 1.00 | 53.25 | A |
| ATOM | 2835 | CG1 | ILE | A | 412 | −41.184 | −23.051 | 25.487 | 1.00 | 52.88 | A |
| ATOM | 2836 | CD1 | ILE | A | 412 | −41.208 | −23.771 | 26.800 | 1.00 | 52.16 | A |
| ATOM | 2837 | C | ILE | A | 412 | −44.410 | −20.960 | 25.317 | 1.00 | 54.70 | A |
| ATOM | 2838 | O | ILE | A | 412 | −45.144 | −20.692 | 26.264 | 1.00 | 54.20 | A |
| ATOM | 2839 | N | VAL | A | 413 | −44.290 | −20.179 | 24.253 | 1.00 | 53.42 | A |
| ATOM | 2840 | CA | VAL | A | 413 | −45.030 | −18.941 | 24.161 | 1.00 | 54.36 | A |
| ATOM | 2841 | CB | VAL | A | 413 | −44.667 | −18.174 | 22.866 | 1.00 | 54.43 | A |
| ATOM | 2842 | CG1 | VAL | A | 413 | −45.751 | −17.129 | 22.540 | 1.00 | 53.39 | A |
| ATOM | 2843 | CG2 | VAL | A | 413 | −43.311 | −17.483 | 23.053 | 1.00 | 53.66 | A |
| ATOM | 2844 | C | VAL | A | 413 | −46.535 | −19.164 | 24.237 | 1.00 | 54.92 | A |
| ATOM | 2845 | O | VAL | A | 413 | −47.221 | −18.507 | 25.009 | 1.00 | 56.61 | A |
| ATOM | 2846 | N | THR | A | 414 | −47.050 | −20.088 | 23.440 | 1.00 | 55.54 | A |
| ATOM | 2847 | CA | THR | A | 414 | −48.484 | −20.372 | 23.441 | 1.00 | 55.90 | A |
| ATOM | 2848 | CB | THR | A | 414 | −48.813 | −21.531 | 22.458 | 1.00 | 58.29 | A |
| ATOM | 2849 | OG1 | THR | A | 414 | −48.720 | −21.048 | 21.110 | 1.00 | 60.35 | A |
| ATOM | 2850 | CG2 | THR | A | 414 | −50.213 | −22.082 | 22.697 | 1.00 | 59.49 | A |
| ATOM | 2851 | C | THR | A | 414 | −48.968 | −20.737 | 24.839 | 1.00 | 54.36 | A |
| ATOM | 2852 | O | THR | A | 414 | −49.966 | −20.200 | 25.334 | 1.00 | 53.06 | A |
| ATOM | 2853 | N | SER | A | 415 | −48.242 | −21.652 | 25.468 | 1.00 | 53.77 | A |
| ATOM | 2854 | CA | SER | A | 415 | −48.567 | −22.115 | 26.803 | 1.00 | 51.90 | A |
| ATOM | 2855 | CB | SER | A | 415 | −47.468 | −23.002 | 27.332 | 1.00 | 48.34 | A |
| ATOM | 2856 | OG | SER | A | 415 | −47.808 | −23.379 | 28.636 | 1.00 | 49.88 | A |
| ATOM | 2857 | C | SER | A | 415 | −48.790 | −20.980 | 27.783 | 1.00 | 52.29 | A |
| ATOM | 2858 | O | SER | A | 415 | −49.784 | −20.963 | 28.499 | 1.00 | 53.98 | A |
| ATOM | 2859 | N | ASP | A | 416 | −47.864 | −20.036 | 27.833 | 1.00 | 51.93 | A |
| ATOM | 2860 | CA | ASP | A | 416 | −48.022 | −18.911 | 28.736 | 1.00 | 52.36 | A |
| ATOM | 2861 | CB | ASP | A | 416 | −46.727 | −18.104 | 28.839 | 1.00 | 53.84 | A |
| ATOM | 2862 | CG | ASP | A | 416 | −45.672 | −18.810 | 29.669 | 1.00 | 57.59 | A |
| ATOM | 2863 | OD1 | ASP | A | 416 | −44.644 | −18.168 | 29.978 | 1.00 | 56.93 | A |
| ATOM | 2864 | OD2 | ASP | A | 416 | −45.871 | −20.007 | 30.008 | 1.00 | 59.68 | A |
| ATOM | 2865 | C | ASP | A | 416 | −49.157 | −18.009 | 28.288 | 1.00 | 51.96 | A |
| ATOM | 2866 | O | ASP | A | 416 | −49.969 | −17.579 | 29.110 | 1.00 | 52.25 | A |
| ATOM | 2867 | N | LEU | A | 417 | −49.225 | −17.730 | 26.990 | 1.00 | 50.31 | A |
| ATOM | 2868 | CA | LEU | A | 417 | −50.274 | −16.871 | 26.474 | 1.00 | 50.04 | A |
| ATOM | 2869 | CB | LEU | A | 417 | −50.065 | −16.619 | 24.990 | 1.00 | 48.41 | A |
| ATOM | 2870 | CG | LEU | A | 417 | −48.919 | −15.644 | 24.736 | 1.00 | 48.67 | A |
| ATOM | 2871 | CD1 | LEU | A | 417 | −48.804 | −15.321 | 23.253 | 1.00 | 47.44 | A |
| ATOM | 2872 | CD2 | LEU | A | 417 | −49.161 | −14.378 | 25.543 | 1.00 | 48.97 | A |
| ATOM | 2873 | C | LEU | A | 417 | −51.668 | −17.428 | 26.734 | 1.00 | 50.89 | A |
| ATOM | 2874 | O | LEU | A | 417 | −52.638 | −16.681 | 26.902 | 1.00 | 50.61 | A |
| ATOM | 2875 | N | LYS | A | 418 | −51.775 | −18.742 | 26.777 | 1.00 | 51.25 | A |
| ATOM | 2876 | CA | LYS | A | 418 | −53.061 | −19.348 | 27.053 | 1.00 | 53.00 | A |
| ATOM | 2877 | CB | LYS | A | 418 | −52.968 | −20.858 | 26.836 | 1.00 | 53.13 | A |
| ATOM | 2878 | CG | LYS | A | 418 | −54.284 | −21.591 | 27.023 | 1.00 | 53.79 | A |
| ATOM | 2879 | CD | LYS | A | 418 | −54.064 | −23.088 | 27.196 | 1.00 | 52.77 | A |
| ATOM | 2880 | CE | LYS | A | 418 | −55.388 | −23.815 | 27.385 | 1.00 | 53.20 | A |
| ATOM | 2881 | NZ | LYS | A | 418 | −55.174 | −25.257 | 27.678 | 1.00 | 51.74 | A |
| ATOM | 2882 | C | LYS | A | 418 | −53.458 | −19.047 | 28.515 | 1.00 | 54.00 | A |
| ATOM | 2883 | O | LYS | A | 418 | −54.556 | −18.543 | 28.799 | 1.00 | 54.34 | A |
| ATOM | 2884 | N | GLN | A | 419 | −52.542 | −19.351 | 29.433 | 1.00 | 54.22 | A |
| ATOM | 2885 | CA | GLN | A | 419 | −52.751 | −19.157 | 30.865 | 1.00 | 53.87 | A |
| ATOM | 2886 | CB | GLN | A | 419 | −51.633 | −19.849 | 31.637 | 1.00 | 53.47 | A |
| ATOM | 2887 | CG | GLN | A | 419 | −51.606 | −19.513 | 33.094 | 1.00 | 56.37 | A |
| ATOM | 2888 | CD | GLN | A | 419 | −50.627 | −20.366 | 33.866 | 1.00 | 59.33 | A |
| ATOM | 2889 | OE1 | GLN | A | 419 | −49.564 | −20.730 | 33.359 | 1.00 | 61.03 | A |
| ATOM | 2890 | NE2 | GLN | A | 419 | −50.970 | −20.677 | 35.112 | 1.00 | 60.61 | A |
| ATOM | 2891 | C | GLN | A | 419 | −52.851 | −17.703 | 31.306 | 1.00 | 53.92 | A |
| ATOM | 2892 | O | GLN | A | 419 | −53.522 | −17.392 | 32.291 | 1.00 | 54.23 | A |
| ATOM | 2893 | N | LEU | A | 420 | −52.191 | −16.812 | 30.579 | 1.00 | 53.43 | A |
| ATOM | 2894 | CA | LEU | A | 420 | −52.218 | −15.408 | 30.936 | 1.00 | 52.81 | A |
| ATOM | 2895 | CB | LEU | A | 420 | −50.880 | −14.743 | 30.574 | 1.00 | 51.39 | A |
| ATOM | 2896 | CG | LEU | A | 420 | −49.653 | −15.090 | 31.435 | 1.00 | 48.97 | A |
| ATOM | 2897 | CD1 | LEU | A | 420 | −48.406 | −14.565 | 30.790 | 1.00 | 46.28 | A |
| ATOM | 2898 | CD2 | LEU | A | 420 | −49.809 | −14.506 | 32.825 | 1.00 | 47.20 | A |
| ATOM | 2899 | C | LEU | A | 420 | −53.366 | −14.666 | 30.281 | 1.00 | 54.28 | A |
| ATOM | 2900 | O | LEU | A | 420 | −54.125 | −13.983 | 30.963 | 1.00 | 56.16 | A |
| ATOM | 2901 | N | LEU | A | 421 | −53.518 | −14.815 | 28.969 | 1.00 | 55.66 | A |
| ATOM | 2902 | CA | LEU | A | 421 | −54.568 | −14.100 | 28.253 | 1.00 | 56.84 | A |
| ATOM | 2903 | CB | LEU | A | 421 | −53.976 | −13.361 | 27.054 | 1.00 | 53.84 | A |
| ATOM | 2904 | CG | LEU | A | 421 | −52.668 | −12.627 | 27.305 | 1.00 | 53.40 | A |
| ATOM | 2905 | CD1 | LEU | A | 421 | −52.335 | −11.786 | 26.109 | 1.00 | 53.84 | A |
| ATOM | 2906 | CD2 | LEU | A | 421 | −52.790 | −11.752 | 28.532 | 1.00 | 53.94 | A |
| ATOM | 2907 | C | LEU | A | 421 | −55.717 | −14.956 | 27.763 | 1.00 | 59.21 | A |
| ATOM | 2908 | O | LEU | A | 421 | −56.676 | −14.435 | 27.191 | 1.00 | 60.30 | A |
| ATOM | 2909 | N | GLY | A | 422 | −55.646 | −16.260 | 27.990 | 1.00 | 60.88 | A |
| ATOM | 2910 | CA | GLY | A | 422 | −56.707 | −17.114 | 27.497 | 1.00 | 62.79 | A |
| ATOM | 2911 | C | GLY | A | 422 | −56.609 | −17.223 | 25.978 | 1.00 | 64.76 | A |
| ATOM | 2912 | O | GLY | A | 422 | −57.611 | −17.135 | 25.268 | 1.00 | 65.00 | A |
| ATOM | 2913 | N | ALA | A | 423 | −55.387 | −17.382 | 25.473 | 1.00 | 65.72 | A |
| ATOM | 2914 | CA | ALA | A | 423 | −55.157 | −17.538 | 24.041 | 1.00 | 65.99 | A |
| ATOM | 2915 | CB | ALA | A | 423 | −53.701 | −17.266 | 23.705 | 1.00 | 65.25 | A |
| ATOM | 2916 | C | ALA | A | 423 | −55.503 | −18.984 | 23.708 | 1.00 | 66.80 | A |
| ATOM | 2917 | O | ALA | A | 423 | −54.979 | −19.910 | 24.327 | 1.00 | 65.80 | A |
| ATOM | 2918 | N | GLU | A | 424 | −56.389 | −19.172 | 22.734 | 1.00 | 68.13 | A |
| ATOM | 2919 | CA | GLU | A | 424 | −56.822 | −20.507 | 22.324 | 1.00 | 68.29 | A |
| ATOM | 2920 | CB | GLU | A | 424 | −58.349 | −20.545 | 22.206 | 1.00 | 69.54 | A |
| ATOM | 2921 | CG | GLU | A | 424 | −59.107 | −20.314 | 23.509 | 1.00 | 71.11 | A |
| ATOM | 2922 | CD | GLU | A | 424 | −60.575 | −20.008 | 23.275 | .00 | 72.08 | A |
| ATOM | 2923 | OE1 | GLU | A | 424 | −60.875 | −19.002 | 22.595 | .00 | 72.53 | A |
| ATOM | 2924 | OE2 | GLU | A | 424 | −61.430 | −20.770 | 23.770 | .00 | 72.54 | A |
| ATOM | 2925 | C | GLU | A | 424 | −56.205 | −20.927 | 20.992 | 1.00 | 67.68 | A |
| ATOM | 2926 | O | GLU | A | 424 | −56.202 | −20.161 | 20.027 | 1.00 | 67.32 | A |
| ATOM | 2927 | N | GLY | A | 425 | −55.675 | −22.143 | 20.949 | 1.00 | 66.71 | A |
| ATOM | 2928 | CA | GLY | A | 425 | −55.086 | −22.642 | 19.719 | 1.00 | 68.87 | A |
| ATOM | 2929 | C | GLY | A | 425 | −53.648 | −22.262 | 19.403 | 1.00 | 69.46 | A |
| ATOM | 2930 | O | GLY | A | 425 | −52.906 | −21.792 | 20.271 | 1.00 | 70.85 | A |
| ATOM | 2931 | N | GLU | A | 426 | −53.253 | −22.473 | 18.147 | 1.00 | 68.95 | A |
| ATOM | 2932 | CA | GLU | A | 426 | −51.900 | −22.171 | 17.706 | 1.00 | 67.97 | A |
| ATOM | 2933 | CB | GLU | A | 426 | −51.465 | −23.129 | 16.598 | 1.00 | 70.20 | A |
| ATOM | 2934 | CG | GLU | A | 426 | −51.388 | −24.591 | 17.031 | 1.00 | 74.36 | A |
| ATOM | 2935 | CD | GLU | A | 426 | −50.654 | −24.799 | 18.359 | 1.00 | 77.31 | A |
| ATOM | 2936 | OE1 | GLU | A | 426 | −51.245 | −24.494 | 19.426 | 1.00 | 77.69 | A |
| ATOM | 2937 | OE2 | GLU | A | 426 | −49.489 | −25.269 | 18.332 | 1.00 | 79.32 | A |
| ATOM | 2938 | C | GLU | A | 426 | −51.766 | −20.742 | 17.236 | 1.00 | 65.93 | A |
| ATOM | 2939 | O | GLU | A | 426 | −52.698 | −20.163 | 16.708 | 1.00 | 65.84 | A |
| ATOM | 2940 | N | PRO | A | 427 | −50.578 | −20.164 | 17.411 | 1.00 | 65.35 | A |
| ATOM | 2941 | CD | PRO | A | 427 | −49.430 | −20.883 | 17.992 | 1.00 | 64.97 | A |
| ATOM | 2942 | CA | PRO | A | 427 | −50.196 | −18.798 | 17.058 | 1.00 | 66.01 | A |
| ATOM | 2943 | CB | PRO | A | 427 | −48.683 | −18.802 | 17.269 | 1.00 | 65.73 | A |
| ATOM | 2944 | CG | PRO | A | 427 | −48.510 | −19.755 | 18.379 | 1.00 | 65.96 | A |
| ATOM | 2945 | C | PRO | A | 427 | −50.561 | −18.182 | 15.713 | 1.00 | 66.89 | A |
| ATOM | 2946 | O | PRO | A | 427 | −50.289 | −16.999 | 15.523 | 1.00 | 69.00 | A |
| ATOM | 2947 | N | THR | A | 428 | −51.144 | −18.931 | 14.778 | 1.00 | 65.90 | A |
| ATOM | 2948 | CA | THR | A | 428 | −51.479 | −18.350 | 13.465 | 1.00 | 66.09 | A |
| ATOM | 2949 | CB | THR | A | 428 | −52.434 | −17.136 | 13.592 | 1.00 | 64.74 | A |
| ATOM | 2950 | OG1 | THR | A | 428 | −53.787 | −17.591 | 13.642 | 1.00 | 67.00 | A |
| ATOM | 2951 | CG2 | THR | A | 428 | −52.282 | −16.196 | 12.422 | 1.00 | 63.15 | A |
| ATOM | 2952 | C | THR | A | 428 | −50.249 | −17.878 | 12.683 | 1.00 | 67.08 | A |
| ATOM | 2953 | O | THR | A | 428 | −50.297 | −17.768 | 11.457 | 1.00 | 69.42 | A |
| ATOM | 2954 | N | TYR | A | 429 | −49.164 | −17.585 | 13.398 | 1.00 | 67.35 | A |
| ATOM | 2955 | CA | TYR | A | 429 | −47.901 | −17.131 | 12.806 | 1.00 | 66.97 | A |
| ATOM | 2956 | CB | TYR | A | 429 | −48.062 | −15.742 | 12.173 | 1.00 | 67.48 | A |
| ATOM | 2957 | CG | TYR | A | 429 | −46.794 | −15.239 | 11.509 | 1.00 | 69.21 | A |
| ATOM | 2958 | CD1 | TYR | A | 429 | −46.359 | −15.779 | 10.299 | 1.00 | 70.11 | A |
| ATOM | 2959 | CE1 | TYR | A | 429 | −45.165 | −15.364 | 9.704 | 1.00 | 70.88 | A |
| ATOM | 2960 | CD2 | TYR | A | 429 | −46.004 | −14.261 | 12.114 | 1.00 | 69.78 | A |
| ATOM | 2961 | CE2 | TYR | A | 429 | −44.806 | −13.836 | 11.526 | 1.00 | 71.24 | A |
| ATOM | 2962 | CZ | TYR | A | 429 | −44.392 | −14.398 | 10.318 | 1.00 | 71.40 | A |
| ATOM | 2963 | OH | TYR | A | 429 | −43.202 | −14.025 | 9.725 | 1.00 | 71.51 | A |
| ATOM | 2964 | C | TYR | A | 429 | −46.820 | −17.072 | 13.893 | 1.00 | 65.50 | A |
| ATOM | 2965 | O | TYR | A | 429 | −47.076 | −16.606 | 15.002 | 1.00 | 65.36 | A |
| ATOM | 2966 | N | VAL | A | 430 | −45.617 | −17.536 | 13.578 | 1.00 | 63.85 | A |
| ATOM | 2967 | CA | VAL | A | 430 | −44.539 | −17.524 | 14.556 | 1.00 | 62.44 | A |
| ATOM | 2968 | CB | VAL | A | 430 | −44.411 | −18.878 | 15.273 | 1.00 | 61.91 | A |
| ATOM | 2969 | CG1 | VAL | A | 430 | −43.441 | −18.759 | 16.418 | 1.00 | 62.02 | A |
| ATOM | 2970 | CG2 | VAL | A | 430 | −45.754 | −19.342 | 15.767 | 1.00 | 61.94 | A |
| ATOM | 2971 | C | VAL | A | 430 | −43.189 | −17.242 | 13.929 | 1.00 | 62.67 | A |
| ATOM | 2972 | O | VAL | A | 430 | −42.826 | −17.843 | 12.921 | 1.00 | 64.62 | A |
| ATOM | 2973 | N | ASN | A | 431 | −42.442 | −16.333 | 14.538 | 1.00 | 62.21 | A |
| ATOM | 2974 | CA | ASN | A | 431 | −41.104 | −15.998 | 14.066 | 1.00 | 61.54 | A |
| ATOM | 2975 | CB | ASN | A | 431 | −41.160 | −14.791 | 13.134 | 1.00 | 62.69 | A |
| ATOM | 2976 | CG | ASN | A | 431 | −40.094 | −14.836 | 12.051 | 1.00 | 63.32 | A |
| ATOM | 2977 | OD1 | ASN | A | 431 | −38.914 | −15.086 | 12.322 | 1.00 | 63.63 | A |
| ATOM | 2978 | ND2 | ASN | A | 431 | −40.508 | −14.585 | 10.813 | 1.00 | 62.70 | A |
| ATOM | 2979 | C | ASN | A | 431 | −40.290 | −15.663 | 15.314 | 1.00 | 60.21 | A |
| ATOM | 2980 | O | ASN | A | 431 | −40.858 | −15.445 | 16.377 | 1.00 | 61.47 | A |
| ATOM | 2981 | N | HIS | A | 432 | −38.972 | −15.616 | 15.210 | 1.00 | 58.22 | A |
| ATOM | 2982 | CA | HIS | A | 432 | −38.190 | −15.297 | 16.392 | 1.00 | 57.34 | A |
| ATOM | 2983 | CB | HIS | A | 432 | −38.250 | −16.447 | 17.375 | 1.00 | 57.95 | A |
| ATOM | 2984 | CG | HIS | A | 432 | −37.302 | −17.552 | 17.040 | 1.00 | 59.29 | A |
| ATOM | 2985 | CD2 | HIS | A | 432 | −36.189 | −17.992 | 17.672 | 1.00 | 60.48 | A |
| ATOM | 2986 | ND1 | HIS | A | 432 | −37.440 | −18.336 | 15.915 | 1.00 | 58.88 | A |
| ATOM | 2987 | CE1 | HIS | A | 432 | −36.457 | −19.217 | 15.872 | 1.00 | 58.67 | A |
| ATOM | 2988 | NE2 | HIS | A | 432 | −35.684 | −19.031 | 16.926 | 1.00 | 59.83 | A |
| ATOM | 2989 | C | HIS | A | 432 | −36.736 | −15.060 | 16.060 | 1.00 | 56.29 | A |
| ATOM | 2990 | O | HIS | A | 432 | −36.262 | −15.465 | 15.011 | 1.00 | 58.55 | A |
| ATOM | 2991 | N | LEU | A | 433 | −36.024 | −14.415 | 16.967 | 1.00 | 54.33 | A |
| ATOM | 2992 | CA | LEU | A | 433 | −34.609 | −14.167 | 16.769 | 1.00 | 53.72 | A |
| ATOM | 2993 | CB | LEU | A | 433 | −34.330 | −12.706 | 16.418 | 1.00 | 54.11 | A |
| ATOM | 2994 | CG | LEU | A | 433 | −32.862 | −12.450 | 16.068 | 1.00 | 54.98 | A |
| ATOM | 2995 | CD1 | LEU | A | 433 | −32.515 | −13.258 | 14.819 | 1.00 | 54.74 | A |
| ATOM | 2996 | CD2 | LEU | A | 433 | −32.611 | −10.961 | 15.840 | 1.00 | 53.69 | A |
| ATOM | 2997 | C | LEU | A | 433 | −33.948 | −14.504 | 18.082 | 1.00 | 52.98 | A |
| ATOM | 2998 | O | LEU | A | 433 | −34.469 | −14.174 | 19.141 | 1.00 | 52.97 | A |
| ATOM | 2999 | N | TYR | A | 434 | −32.807 | −15.168 | 18.011 | 1.00 | 52.79 | A |
| ATOM | 3000 | CA | TYR | A | 434 | −32.094 | −15.560 | 19.207 | 1.00 | 52.83 | A |
| ATOM | 3001 | CB | TYR | A | 434 | −31.715 | −17.038 | 19.128 | 1.00 | 52.75 | A |
| ATOM | 3002 | CG | TYR | A | 434 | −30.651 | −17.468 | 20.121 | 1.00 | 55.50 | A |
| ATOM | 3003 | CD1 | TYR | A | 434 | −30.882 | −17.423 | 21.495 | 1.00 | 57.37 | A |
| ATOM | 3004 | CE1 | TYR | A | 434 | −29.908 | −17.860 | 22.413 | 1.00 | 58.71 | A |
| ATOM | 3005 | CD2 | TYR | A | 434 | −29.419 | −17.953 | 19.681 | 1.00 | 56.94 | A |
| ATOM | 3006 | CE2 | TYR | A | 434 | −28.439 | −18.395 | 20.583 | 1.00 | 59.00 | A |
| ATOM | 3007 | CZ | TYR | A | 434 | −28.687 | −18.349 | 21.948 | 1.00 | 59.85 | A |
| ATOM | 3008 | OH | TYR | A | 434 | −27.726 | −18.808 | 22.834 | 1.00 | 59.01 | A |
| ATOM | 3009 | C | TYR | A | 434 | −30.849 | −14.726 | 19.430 | 1.00 | 52.50 | A |
| ATOM | 3010 | O | TYR | A | 434 | −29.971 | −14.668 | 18.569 | 1.00 | 52.90 | A |
| ATOM | 3011 | N | TRP | A | 435 | −30.789 | −14.077 | 20.589 | 1.00 | 51.08 | A |
| ATOM | 3012 | CA | TRP | A | 435 | −29.632 | −13.278 | 20.957 | 1.00 | 50.93 | A |
| ATOM | 3013 | CB | TRP | A | 435 | −30.036 | −11.927 | 21.546 | 1.00 | 47.93 | A |
| ATOM | 3014 | CG | TRP | A | 435 | −30.892 | −11.077 | 20.678 | 1.00 | 45.96 | A |
| ATOM | 3015 | CD2 | TRP | A | 435 | −30.454 | −10.099 | 19.728 | 1.00 | 45.86 | A |
| ATOM | 3016 | CE2 | TRP | A | 435 | −31.611 | −9.523 | 19.160 | 1.00 | 44.63 | A |
| ATOM | 3017 | CE3 | TRP | A | 435 | −29.196 | −9.653 | 19.302 | 1.00 | 46.06 | A |
| ATOM | 3018 | CD1 | TRP | A | 435 | −32.250 | −11.056 | 20.648 | 1.00 | 45.44 | A |
| ATOM | 3019 | NE1 | TRP | A | 435 | −32.693 | −10.124 | 19.741 | 1.00 | 44.83 | A |
| ATOM | 3020 | CZ2 | TRP | A | 435 | −31.552 | −8.528 | 18.189 | 1.00 | 43.35 | A |
| ATOM | 3021 | CZ3 | TRP | A | 435 | −29.136 | −8.662 | 18.335 | 1.00 | 44.85 | A |
| ATOM | 3022 | CH2 | TRP | A | 435 | −30.310 | −8.111 | 17.790 | 1.00 | 45.86 | A |
| ATOM | 3023 | C | TRP | A | 435 | −28.872 | −14.051 | 22.028 | 1.00 | 51.53 | A |
| ATOM | 3024 | O | TRP | A | 435 | −29.353 | −14.178 | 23.150 | 1.00 | 51.86 | A |
| ATOM | 3025 | N | SER | A | 436 | −27.693 | −14.564 | 21.688 | 1.00 | 52.23 | A |
| ATOM | 3026 | CA | SER | A | 436 | −26.899 | −15.311 | 22.658 | 1.00 | 52.71 | A |
| ATOM | 3027 | CB | SER | A | 436 | −25.772 | −16.052 | 21.953 | 1.00 | 54.89 | A |
| ATOM | 3028 | OG | SER | A | 436 | −24.898 | −15.124 | 21.327 | 1.00 | 58.93 | A |
| ATOM | 3029 | C | SER | A | 436 | −26.302 | −14.377 | 23.713 | 1.00 | 50.75 | A |
| ATOM | 3030 | O | SER | A | 436 | −25.899 | −14.826 | 24.779 | 1.00 | 49.41 | A |
| ATOM | 3031 | N | LYS | A | 437 | −26.228 | −13.088 | 23.393 | 1.00 | 48.88 | A |
| ATOM | 3032 | CA | LYS | A | 437 | −25.697 | −12.086 | 24.312 | 1.00 | 49.16 | A |
| ATOM | 3033 | CB | LYS | A | 437 | −24.223 | −11.815 | 24.020 | 1.00 | 50.27 | A |
| ATOM | 3034 | CG | LYS | A | 437 | −23.354 | −13.025 | 24.258 | 1.00 | 54.74 | A |
| ATOM | 3035 | CD | LYS | A | 437 | −21.879 | −12.704 | 24.165 | 1.00 | 58.11 | A |
| ATOM | 3036 | CE | LYS | A | 437 | −21.066 | −13.997 | 24.169 | 1.00 | 60.60 | A |
| ATOM | 3037 | NZ | LYS | A | 437 | −21.397 | −14.852 | 22.973 | 1.00 | 62.53 | A |
| ATOM | 3038 | C | LYS | A | 437 | −26.506 | −10.795 | 24.206 | 1.00 | 48.24 | A |
| ATOM | 3039 | O | LYS | A | 437 | −26.019 | −9.767 | 23.730 | 1.00 | 46.02 | A |
| ATOM | 3040 | N | ALA | A | 438 | −27.747 | −10.876 | 24.679 | 1.00 | 47.85 | A |
| ATOM | 3041 | CA | ALA | A | 438 | −28.705 | −9.783 | 24.655 | 1.00 | 46.75 | A |
| ATOM | 3042 | CB | ALA | A | 438 | −30.065 | −10.305 | 25.054 | 1.00 | 47.90 | A |
| ATOM | 3043 | C | ALA | A | 438 | −28.354 | −8.592 | 25.521 | 1.00 | 46.97 | A |
| ATOM | 3044 | O | ALA | A | 438 | −28.465 | −7.462 | 25.067 | 1.00 | 45.99 | A |
| ATOM | 3045 | N | PHE | A | 439 | −27.967 | −8.837 | 26.773 | 1.00 | 47.32 | A |
| ATOM | 3046 | CA | PHE | A | 439 | −27.621 | −7.750 | 27.691 | 1.00 | 47.74 | A |
| ATOM | 3047 | CB | PHE | A | 439 | −28.749 | −7.461 | 28.688 | 1.00 | 47.02 | A |
| ATOM | 3048 | CG | PHE | A | 439 | −30.022 | −6.968 | 28.079 | 1.00 | 44.00 | A |
| ATOM | 3049 | CD1 | PHE | A | 439 | −30.969 | −7.858 | 27.608 | 1.00 | 43.57 | A |
| ATOM | 3050 | CD2 | PHE | A | 439 | −30.305 | −5.607 | 28.032 | 1.00 | 43.73 | A |
| ATOM | 3051 | CE1 | PHE | A | 439 | −32.178 | −7.412 | 27.105 | 1.00 | 40.25 | A |
| ATOM | 3052 | CE2 | PHE | A | 439 | −31.512 | −5.150 | 27.530 | 1.00 | 39.65 | A |
| ATOM | 3053 | CZ | PHE | A | 439 | −32.448 | −6.062 | 27.069 | 1.00 | 40.53 | A |
| ATOM | 3054 | C | PHE | A | 439 | −26.406 | −8.067 | 28.540 | 1.00 | 49.73 | A |
| ATOM | 3055 | O | PHE | A | 439 | −26.218 | −9.208 | 28.968 | 1.00 | 49.35 | A |
| ATOM | 3056 | N | PRO | A | 440 | −25.591 | −7.043 | 28.842 | 1.00 | 50.86 | A |
| ATOM | 3057 | CD | PRO | A | 440 | −25.709 | −5.678 | 28.327 | 1.00 | 50.70 | A |
| ATOM | 3058 | CA | PRO | A | 440 | −24.383 | −7.168 | 29.659 | 1.00 | 52.59 | A |
| ATOM | 3059 | CB | PRO | A | 440 | −23.703 | −5.813 | 29.503 | 1.00 | 49.43 | A |
| ATOM | 3060 | CG | PRO | A | 440 | −24.271 | −5.269 | 28.269 | 1.00 | 50.81 | A |
| ATOM | 3061 | C | PRO | A | 440 | −24.846 | −7.380 | 31.088 | 1.00 | 55.28 | A |
| ATOM | 3062 | O | PRO | A | 440 | −25.724 | −6.674 | 31.572 | 1.00 | 56.43 | A |
| ATOM | 3063 | N | LEU | A | 441 | −24.265 | −8.345 | 31.771 | 1.00 | 58.33 | A |
| ATOM | 3064 | CA | LEU | A | 441 | −24.677 | −8.585 | 33.132 | 1.00 | 62.06 | A |
| ATOM | 3065 | CB | LEU | A | 441 | −24.660 | −10.087 | 33.413 | 1.00 | 61.36 | A |
| ATOM | 3066 | CG | LEU | A | 441 | −25.820 | −10.588 | 34.275 | 1.00 | 62.10 | A |
| ATOM | 3067 | CD1 | LEU | A | 441 | −27.096 | −9.866 | 33.863 | 1.00 | 61.86 | A |
| ATOM | 3068 | CD2 | LEU | A | 441 | −25.979 | −12.096 | 34.131 | 1.00 | 60.83 | A |
| ATOM | 3069 | C | LEU | A | 441 | −23.784 | −7.829 | 34.112 | 1.00 | 66.23 | A |
| ATOM | 3070 | O | LEU | A | 441 | −22.635 | −8.220 | 34.363 | 1.00 | 67.78 | A |
| ATOM | 3071 | N | TYR | A | 442 | −24.311 | −6.724 | 34.637 | 1.00 | 69.01 | A |
| ATOM | 3072 | CA | TYR | A | 442 | −23.595 | −5.911 | 35.612 | 1.00 | 71.58 | A |
| ATOM | 3073 | CB | TYR | A | 442 | −24.352 | −4.609 | 35.914 | 1.00 | 72.73 | A |
| ATOM | 3074 | CG | TYR | A | 442 | −24.424 | −3.611 | 34.782 | 1.00 | 73.63 | A |
| ATOM | 3075 | CD1 | TYR | A | 442 | −25.153 | −2.435 | 34.917 | 1.00 | 73.91 | A |
| ATOM | 3076 | CE1 | TYR | A | 442 | −25.215 | −1.500 | 33.874 | 1.00 | 75.91 | A |
| ATOM | 3077 | CD2 | TYR | A | 442 | −23.756 | −3.838 | 33.578 | 1.00 | 75.58 | A |
| ATOM | 3078 | CE2 | TYR | A | 442 | −23.805 | −2.916 | 32.530 | 1.00 | 75.65 | A |
| ATOM | 3079 | CZ | TYR | A | 442 | −24.533 | −1.749 | 32.682 | 1.00 | 76.48 | A |
| ATOM | 3080 | OH | TYR | A | 442 | −24.546 | −.833 | 31.647 | 1.00 | 76.14 | A |
| ATOM | 3081 | C | TYR | A | 442 | −23.574 | −6.757 | 36.866 | 1.00 | 73.58 | A |
| ATOM | 3082 | O | TYR | A | 442 | −24.458 | −6.632 | 37.705 | 1.00 | 74.15 | A |
| ATOM | 3083 | N | GLY | A | 443 | −22.571 | −7.620 | 36.989 | 1.00 | 76.54 | A |
| ATOM | 3084 | CA | GLY | A | 443 | −22.479 | −8.494 | 38.149 | 1.00 | 79.15 | A |
| ATOM | 3085 | C | GLY | A | 443 | −21.681 | −7.959 | 39.321 | 1.00 | 80.78 | A |
| ATOM | 3086 | O | GLY | A | 443 | −21.543 | −6.750 | 39.496 | 1.00 | 81.04 | A |
| ATOM | 3087 | N | ALA | A | 444 | −21.151 | −8.875 | 40.125 | 1.00 | 82.87 | A |
| ATOM | 3088 | CA | ALA | A | 444 | −20.366 | −8.519 | 41.304 | 1.00 | 84.89 | A |
| ATOM | 3089 | CB | ALA | A | 444 | −20.029 | −9.779 | 42.101 | 1.00 | 83.78 | A |
| ATOM | 3090 | C | ALA | A | 444 | −19.084 | −7.757 | 40.963 | 1.00 | 86.83 | A |
| ATOM | 3091 | O | ALA | A | 444 | −18.889 | −6.618 | 41.400 | 1.00 | 87.64 | A |
| ATOM | 3092 | N | ASN | A | 445 | −18.212 | −8.386 | 40.182 | 1.00 | 88.30 | A |
| ATOM | 3093 | CA | ASN | A | 445 | −16.950 | −7.772 | 39.800 | 1.00 | 89.37 | A |
| ATOM | 3094 | CB | ASN | A | 445 | −15.999 | −8.839 | 39.264 | 1.00 | 91.41 | A |
| ATOM | 3095 | CG | ASN | A | 445 | −16.491 | −9.461 | 37.969 | 1.00 | 93.34 | A |
| ATOM | 3096 | OD1 | ASN | A | 445 | −15.849 | −10.358 | 37.419 | 1.00 | 95.31 | A |
| ATOM | 3097 | ND2 | ASN | A | 445 | −17.633 | −8.988 | 37.473 | 1.00 | 92.95 | A |
| ATOM | 3098 | C | ASN | A | 445 | −17.098 | −6.668 | 38.756 | 1.00 | 89.29 | A |
| ATOM | 3099 | O | ASN | A | 445 | −16.132 | −6.333 | 38.071 | 1.00 | 89.20 | A |
| ATOM | 3100 | N | TYR | A | 446 | −18.291 | −6.096 | 38.627 | 1.00 | 89.58 | A |
| ATOM | 3101 | CA | TYR | A | 446 | −18.479 | −5.041 | 37.638 | 1.00 | 90.56 | A |
| ATOM | 3102 | CB | TYR | A | 446 | −19.969 | −4.817 | 37.346 | 1.00 | 91.11 | A |
| ATOM | 3103 | CG | TYR | A | 446 | −20.232 | −4.723 | 35.856 | 1.00 | 92.41 | A |
| ATOM | 3104 | CD1 | TYR | A | 446 | −20.131 | −5.857 | 35.044 | 1.00 | 92.22 | A |
| ATOM | 3105 | CE1 | TYR | A | 446 | −20.262 | −5.774 | 33.660 | 1.00 | 92.08 | A |
| ATOM | 3106 | CD2 | TYR | A | 446 | −20.483 | −3.494 | 35.240 | 1.00 | 92.60 | A |
| ATOM | 3107 | CE2 | TYR | A | 446 | −20.615 | −3.400 | 33.851 | 1.00 | 92.73 | A |
| ATOM | 3108 | CZ | TYR | A | 446 | −20.501 | −4.548 | 33.068 | 1.00 | 92.26 | A |
| ATOM | 3109 | OH | TYR | A | 446 | −20.613 | −4.476 | 31.697 | 1.00 | 90.82 | A |
| ATOM | 3110 | C | TYR | A | 446 | −17.817 | −3.725 | 38.048 | 1.00 | 90.44 | A |
| ATOM | 3111 | O | TYR | A | 446 | −17.707 | −2.791 | 37.247 | 1.00 | 90.33 | A |
| ATOM | 3112 | N | ASP | A | 447 | −17.374 | −3.661 | 39.298 | 1.00 | 90.57 | A |
| ATOM | 3113 | CA | ASP | A | 447 | −16.712 | −2.476 | 39.818 | 1.00 | 89.85 | A |
| ATOM | 3114 | CB | ASP | A | 447 | −16.581 | −2.559 | 41.346 | 1.00 | 92.38 | A |
| ATOM | 3115 | CG | ASP | A | 447 | −17.914 | −2.822 | 42.044 | 1.00 | 95.02 | A |
| ATOM | 3116 | OD1 | ASP | A | 447 | −17.910 | −3.105 | 43.267 | 1.00 | 96.46 | A |
| ATOM | 3117 | OD2 | ASP | A | 447 | −18.968 | −2.744 | 41.377 | 1.00 | 96.57 | A |
| ATOM | 3118 | C | ASP | A | 447 | −15.328 | −2.463 | 39.190 | 1.00 | 88.35 | A |
| ATOM | 3119 | O | ASP | A | 447 | −14.740 | −1.406 | 38.985 | 1.00 | 88.21 | A |
| ATOM | 3120 | N | SER | A | 448 | −14.827 | −3.654 | 38.875 | 1.00 | 86.82 | A |
| ATOM | 3121 | CA | SER | A | 448 | −13.502 | −3.818 | 38.291 | 1.00 | 86.50 | A |
| ATOM | 3122 | CB | SER | A | 448 | −12.898 | −5.165 | 38.699 | 1.00 | 87.83 | A |
| ATOM | 3123 | OG | SER | A | 448 | −13.475 | −6.234 | 37.962 | 1.00 | 87.39 | A |
| ATOM | 3124 | C | SER | A | 448 | −13.521 | −3.738 | 36.776 | 1.00 | 86.32 | A |
| ATOM | 3125 | O | SER | A | 448 | −12.529 | −3.336 | 36.159 | 1.00 | 86.37 | A |
| ATOM | 3126 | N | VAL | A | 449 | −14.636 | −4.150 | 36.176 | 1.00 | 85.15 | A |
| ATOM | 3127 | CA | VAL | A | 449 | −14.771 | −4.100 | 34.724 | 1.00 | 83.12 | A |
| ATOM | 3128 | CB | VAL | A | 449 | −16.174 | −4.525 | 34.272 | 1.00 | 82.47 | A |
| ATOM | 3129 | CG1 | VAL | A | 449 | −16.296 | −4.387 | 32.773 | 1.00 | 83.53 | A |
| ATOM | 3130 | CG2 | VAL | A | 449 | −16.434 | −5.945 | 34.671 | 1.00 | 82.27 | A |
| ATOM | 3131 | C | VAL | A | 449 | −14.543 | −2.663 | 34.281 | 1.00 | 82.18 | A |
| ATOM | 3132 | O | VAL | A | 449 | −13.754 | −2.389 | 33.377 | 1.00 | 80.55 | A |
| ATOM | 3133 | N | LEU | A | 450 | −15.241 | −1.748 | 34.939 | 1.00 | 81.78 | A |
| ATOM | 3134 | CA | LEU | A | 450 | −15.114 | −.339 | 34.630 | 1.00 | 83.01 | A |
| ATOM | 3135 | CB | LEU | A | 450 | −15.974 | .475 | 35.600 | 1.00 | 84.22 | A |
| ATOM | 3136 | CG | LEU | A | 450 | −17.476 | .155 | 35.629 | 1.00 | 85.37 | A |
| ATOM | 3137 | CD1 | LEU | A | 450 | −18.161 | 1.041 | 36.671 | 1.00 | 85.22 | A |
| ATOM | 3138 | CD2 | LEU | A | 450 | −18.093 | .380 | 34.252 | 1.00 | 86.00 | A |
| ATOM | 3139 | C | LEU | A | 450 | −13.649 | .115 | 34.694 | 1.00 | 82.80 | A |
| ATOM | 3140 | O | LEU | A | 450 | −13.200 | .903 | 33.856 | 1.00 | 82.73 | A |
| ATOM | 3141 | N | ASP | A | 451 | −12.911 | −.392 | 35.680 | 1.00 | 82.32 | A |
| ATOM | 3142 | CA | ASP | A | 451 | −11.500 | −.045 | 35.865 | 1.00 | 81.47 | A |
| ATOM | 3143 | CB | ASP | A | 451 | −10.922 | −.755 | 37.085 | 1.00 | 84.10 | A |
| ATOM | 3144 | CG | ASP | A | 451 | −11.534 | −.286 | 38.377 | 1.00 | 86.81 | A |
| ATOM | 3145 | OD1 | ASP | A | 451 | −11.344 | .896 | 38.742 | 1.00 | 88.20 | A |
| ATOM | 3146 | OD2 | ASP | A | 451 | −12.210 | −1.107 | 39.029 | 1.00 | 89.73 | A |
| ATOM | 3147 | C | ASP | A | 451 | −10.654 | −.421 | 34.668 | 1.00 | 79.72 | A |
| ATOM | 3148 | O | ASP | A | 451 | −9.734 | .302 | 34.292 | 1.00 | 79.31 | A |
| ATOM | 3149 | N | ALA | A | 452 | −10.957 | −1.574 | 34.089 | 1.00 | 77.85 | A |
| ATOM | 3150 | CA | ALA | A | 452 | −10.224 | −2.062 | 32.936 | 1.00 | 76.17 | A |
| ATOM | 3151 | CB | ALA | A | 452 | −10.620 | −3.495 | 32.646 | 1.00 | 76.13 | A |
| ATOM | 3152 | C | ALA | A | 452 | −10.496 | −1.188 | 31.725 | 1.00 | 75.15 | A |
| ATOM | 3153 | O | ALA | A | 452 | −9.581 | −.838 | 30.986 | 1.00 | 74.65 | A |
| ATOM | 3154 | N | ILE | A | 453 | −11.760 | −.841 | 31.520 | 1.00 | 74.94 | A |
| ATOM | 3155 | CA | ILE | A | 453 | −12.129 | −.003 | 30.395 | 1.00 | 73.93 | A |
| ATOM | 3156 | CB | ILE | A | 453 | −13.638 | .327 | 30.391 | 1.00 | 73.31 | A |
| ATOM | 3157 | CG2 | ILE | A | 453 | −13.953 | 1.292 | 29.261 | 1.00 | 72.17 | A |
| ATOM | 3158 | CG1 | ILE | A | 453 | −14.452 | −.959 | 30.238 | 1.00 | 72.52 | A |
| ATOM | 3159 | CD1 | ILE | A | 453 | −15.945 | −.748 | 30.268 | 1.00 | 72.55 | A |
| ATOM | 3160 | C | ILE | A | 453 | −11.340 | 1.286 | 30.496 | 1.00 | 73.61 | A |
| ATOM | 3161 | O | ILE | A | 453 | −10.911 | 1.831 | 29.482 | 1.00 | 75.07 | A |
| ATOM | 3162 | N | ASP | A | 454 | −11.133 | 1.762 | 31.721 | 1.00 | 72.46 | A |
| ATOM | 3163 | CA | ASP | A | 454 | −10.384 | 2.993 | 31.927 | 1.00 | 72.43 | A |
| ATOM | 3164 | CB | ASP | A | 454 | −10.690 | 3.584 | 33.297 | 1.00 | 72.75 | A |
| ATOM | 3165 | CG | ASP | A | 454 | −12.107 | 4.071 | 33.409 | 1.00 | 73.70 | A |
| ATOM | 3166 | OD1 | ASP | A | 454 | −12.579 | 4.726 | 32.456 | 1.00 | 75.73 | A |
| ATOM | 3167 | OD2 | ASP | A | 454 | −12.742 | 3.808 | 34.449 | 1.00 | 73.40 | A |
| ATOM | 3168 | C | ASP | A | 454 | −8.882 | 2.787 | 31.783 | 1.00 | 72.40 | A |
| ATOM | 3169 | O | ASP | A | 454 | −8.179 | 3.645 | 31.250 | 1.00 | 72.43 | A |
| ATOM | 3170 | N | LYS | A | 455 | −8.390 | 1.649 | 32.262 | 1.00 | 72.75 | A |
| ATOM | 3171 | CA | LYS | A | 455 | −6.969 | 1.332 | 32.171 | 1.00 | 72.16 | A |
| ATOM | 3172 | CB | LYS | A | 455 | −6.685 | −.016 | 32.821 | 1.00 | 71.77 | A |
| ATOM | 3173 | CG | LYS | A | 455 | −5.224 | −.414 | 32.803 | .00 | 73.26 | A |
| ATOM | 3174 | CD | LYS | A | 455 | −5.001 | −1.714 | 33.554 | .00 | 73.85 | A |
| ATOM | 3175 | CE | LYS | A | 455 | −5.375 | −1.577 | 35.023 | .00 | 74.37 | A |
| ATOM | 3176 | NZ | LYS | A | 455 | −4.575 | −.520 | 35.700 | .00 | 74.60 | A |
| ATOM | 3177 | C | LYS | A | 455 | −6.573 | 1.283 | 30.709 | 1.00 | 72.71 | A |
| ATOM | 3178 | O | LYS | A | 455 | −5.534 | 1.805 | 30.319 | 1.00 | 72.11 | A |
| ATOM | 3179 | N | MSE | A | 456 | −7.422 | .646 | 29.908 | 1.00 | 73.99 | A |
| ATOM | 3180 | CA | MSE | A | 456 | −7.222 | .511 | 28.472 | 1.00 | 74.46 | A |
| ATOM | 3181 | CB | MSE | A | 456 | −8.336 | −.357 | 27.912 | 1.00 | 75.00 | A |
| ATOM | 3182 | CG | MSE | A | 456 | −8.299 | −.553 | 26.439 | 1.00 | 78.12 | A |
| ATOM | 3183 | SE | MSE | A | 456 | −9.828 | −1.571 | 25.946 | 1.00 | 82.37 | A |
| ATOM | 3184 | CE | MSE | A | 456 | −8.901 | −3.170 | 25.357 | 1.00 | 83.38 | A |
| ATOM | 3185 | C | MSE | A | 456 | −7.215 | 1.891 | 27.792 | 1.00 | 75.02 | A |
| ATOM | 3186 | O | MSE | A | 456 | −6.309 | 2.217 | 27.011 | 1.00 | 73.81 | A |
| ATOM | 3187 | N | GLU | A | 457 | −8.223 | 2.705 | 28.103 | 1.00 | 75.05 | A |
| ATOM | 3188 | CA | GLU | A | 457 | −8.318 | 4.047 | 27.538 | 1.00 | 74.43 | A |
| ATOM | 3189 | CB | GLU | A | 457 | −9.634 | 4.712 | 27.953 | 1.00 | 72.04 | A |
| ATOM | 3190 | CG | GLU | A | 457 | −10.840 | 4.105 | 27.298 | 1.00 | 71.27 | A |
| ATOM | 3191 | CD | GLU | A | 457 | −12.129 | 4.773 | 27.698 | 1.00 | 72.26 | A |
| ATOM | 3192 | OE1 | GLU | A | 457 | −12.454 | 4.777 | 28.908 | 1.00 | 72.12 | A |
| ATOM | 3193 | OE2 | GLU | A | 457 | −12.822 | 5.290 | 26.794 | 1.00 | 73.28 | A |
| ATOM | 3194 | C | GLU | A | 457 | −7.145 | 4.936 | 27.937 | 1.00 | 74.58 | A |
| ATOM | 3195 | O | GLU | A | 457 | −6.773 | 5.831 | 27.187 | 1.00 | 75.09 | A |
| ATOM | 3196 | N | LYS | A | 458 | −6.559 | 4.689 | 29.107 | 1.00 | 75.00 | A |
| ATOM | 3197 | CA | LYS | A | 458 | −5.440 | 5.500 | 29.570 | 1.00 | 76.24 | A |
| ATOM | 3198 | CB | LYS | A | 458 | −5.336 | 5.458 | 31.097 | 1.00 | 77.67 | A |
| ATOM | 3199 | CG | LYS | A | 458 | −6.537 | 6.093 | 31.809 | 1.00 | 80.68 | A |
| ATOM | 3200 | CD | LYS | A | 458 | −6.308 | 6.280 | 33.319 | 1.00 | 82.18 | A |
| ATOM | 3201 | CE | LYS | A | 458 | −7.522 | 6.947 | 33.986 | 1.00 | 82.83 | A |
| ATOM | 3202 | NZ | LYS | A | 458 | −7.334 | 7.249 | 35.440 | 1.00 | 82.04 | A |
| ATOM | 3203 | C | LYS | A | 458 | −4.114 | 5.096 | 28.960 | 1.00 | 76.79 | A |
| ATOM | 3204 | O | LYS | A | 458 | −3.301 | 5.956 | 28.626 | 1.00 | 76.19 | A |
| ATOM | 3205 | N | ASN | A | 459 | −3.908 | 3.790 | 28.793 | 1.00 | 78.18 | A |
| ATOM | 3206 | CA | ASN | A | 459 | −2.664 | 3.260 | 28.227 | 1.00 | 78.65 | A |
| ATOM | 3207 | CB | ASN | A | 459 | −2.398 | 1.861 | 28.772 | 1.00 | 79.98 | A |
| ATOM | 3208 | CG | ASN | A | 459 | −2.252 | 1.847 | 30.278 | 1.00 | 81.05 | A |
| ATOM | 3209 | OD1 | ASN | A | 459 | −1.485 | 2.630 | 30.839 | 1.00 | 80.73 | A |
| ATOM | 3210 | ND2 | ASN | A | 459 | −2.984 | .954 | 30.942 | 1.00 | 81.31 | A |
| ATOM | 3211 | C | ASN | A | 459 | −2.582 | 3.225 | 26.706 | 1.00 | 78.42 | A |
| ATOM | 3212 | O | ASN | A | 459 | −1.498 | 3.059 | 26.152 | 1.00 | 78.57 | A |
| ATOM | 3213 | N | LEU | A | 460 | −3.719 | 3.351 | 26.033 | 1.00 | 78.88 | A |
| ATOM | 3214 | CA | LEU | A | 460 | −3.741 | 3.372 | 24.574 | 1.00 | 79.29 | A |
| ATOM | 3215 | CB | LEU | A | 460 | −4.490 | 2.156 | 24.035 | 1.00 | 79.58 | A |
| ATOM | 3216 | CG | LEU | A | 460 | −3.654 | 1.032 | 23.415 | 1.00 | 79.80 | A |
| ATOM | 3217 | CD1 | LEU | A | 460 | −2.432 | .723 | 24.255 | 1.00 | 79.63 | A |
| ATOM | 3218 | CD2 | LEU | A | 460 | −4.524 | −.187 | 23.270 | 1.00 | 79.46 | A |
| ATOM | 3219 | C | LEU | A | 460 | −4.445 | 4.651 | 24.151 | 1.00 | 79.39 | A |
| ATOM | 3220 | O | LEU | A | 460 | −5.654 | 4.654 | 23.960 | 1.00 | 80.34 | A |
| ATOM | 3221 | N | PRO | A | 461 | −3.694 | 5.758 | 24.001 | 1.00 | 79.16 | A |
| ATOM | 3222 | CD | PRO | A | 461 | −2.235 | 5.812 | 24.170 | 1.00 | 79.90 | A |
| ATOM | 3223 | CA | PRO | A | 461 | −4.203 | 7.081 | 23.610 | 1.00 | 78.93 | A |
| ATOM | 3224 | CB | PRO | A | 461 | −2.936 | 7.933 | 23.530 | 1.00 | 79.01 | A |
| ATOM | 3225 | CG | PRO | A | 461 | −2.018 | 7.274 | 24.485 | 1.00 | 79.15 | A |
| ATOM | 3226 | C | PRO | A | 461 | −4.992 | 7.111 | 22.305 | 1.00 | 77.89 | A |
| ATOM | 3227 | O | PRO | A | 461 | −4.654 | 6.417 | 21.344 | 1.00 | 78.36 | A |
| ATOM | 3228 | N | GLY | A | 462 | −6.040 | 7.929 | 22.275 | 1.00 | 75.76 | A |
| ATOM | 3229 | CA | GLY | A | 462 | −6.857 | 8.025 | 21.079 | 1.00 | 74.22 | A |
| ATOM | 3230 | C | GLY | A | 462 | −8.008 | 7.031 | 21.049 | 1.00 | 72.71 | A |
| ATOM | 3231 | O | GLY | A | 462 | −9.036 | 7.271 | 20.411 | 1.00 | 73.59 | A |
| ATOM | 3232 | N | LEU | A | 463 | −7.833 | 5.910 | 21.739 | 1.00 | 70.00 | A |
| ATOM | 3233 | CA | LEU | A | 463 | −8.852 | 4.880 | 21.800 | 1.00 | 66.70 | A |
| ATOM | 3234 | CB | LEU | A | 463 | −8.221 | 3.535 | 22.131 | 1.00 | 68.06 | A |
| ATOM | 3235 | CG | LEU | A | 463 | −9.210 | 2.369 | 22.161 | 1.00 | 70.15 | A |
| ATOM | 3236 | CD1 | LEU | A | 463 | −9.647 | 2.058 | 20.733 | 1.00 | 71.04 | A |
| ATOM | 3237 | CD2 | LEU | A | 463 | −8.570 | 1.144 | 22.801 | 1.00 | 70.71 | A |
| ATOM | 3238 | C | LEU | A | 463 | −9.863 | 5.225 | 22.873 | 1.00 | 65.02 | A |
| ATOM | 3239 | O | LEU | A | 463 | −9.500 | 5.405 | 24.032 | 1.00 | 64.46 | A |
| ATOM | 3240 | N | PHE | A | 464 | −11.132 | 5.318 | 22.488 | 1.00 | 63.04 | A |
| ATOM | 3241 | CA | PHE | A | 464 | −12.195 | 5.632 | 23.431 | 1.00 | 60.91 | A |
| ATOM | 3242 | CB | PHE | A | 464 | −12.802 | 7.003 | 23.132 | 1.00 | 59.27 | A |
| ATOM | 3243 | CG | PHE | A | 464 | −11.827 | 8.129 | 23.247 | 1.00 | 59.05 | A |
| ATOM | 3244 | CD1 | PHE | A | 464 | −10.897 | 8.367 | 22.244 | 1.00 | 59.25 | A |
| ATOM | 3245 | CD2 | PHE | A | 464 | −11.789 | 8.915 | 24.389 | 1.00 | 59.09 | A |
| ATOM | 3246 | CE1 | PHE | A | 464 | −9.939 | 9.365 | 22.382 | 1.00 | 57.55 | A |
| ATOM | 3247 | CE2 | PHE | A | 464 | −10.832 | 9.915 | 24.532 | 1.00 | 57.72 | A |
| ATOM | 3248 | CZ | PHE | A | 464 | −9.908 | 10.137 | 23.530 | 1.00 | 57.36 | A |
| ATOM | 3249 | C | PHE | A | 464 | −13.275 | 4.574 | 23.344 | 1.00 | 61.53 | A |
| ATOM | 3250 | O | PHE | A | 464 | −13.774 | 4.270 | 22.267 | 1.00 | 61.11 | A |
| ATOM | 3251 | N | TYR | A | 465 | −13.632 | 4.006 | 24.485 | 1.00 | 62.54 | A |
| ATOM | 3252 | CA | TYR | A | 465 | −14.656 | 2.983 | 24.511 | 1.00 | 64.05 | A |
| ATOM | 3253 | CB | TYR | A | 465 | −14.347 | 1.954 | 25.611 | 1.00 | 63.11 | A |
| ATOM | 3254 | CG | TYR | A | 465 | −15.291 | .767 | 25.656 | 1.00 | 63.81 | A |
| ATOM | 3255 | CD1 | TYR | A | 465 | −16.500 | .835 | 26.348 | 1.00 | 65.03 | A |
| ATOM | 3256 | CE1 | TYR | A | 465 | −17.387 | −.248 | 26.365 | 1.00 | 65.58 | A |
| ATOM | 3257 | CD2 | TYR | A | 465 | −14.988 | −.413 | 24.986 | 1.00 | 63.54 | A |
| ATOM | 3258 | CE2 | TYR | A | 465 | −15.861 | −1.497 | 24.998 | 1.00 | 64.37 | A |
| ATOM | 3259 | CZ | TYR | A | 465 | −17.060 | −1.408 | 25.688 | 1.00 | 65.08 | A |
| ATOM | 3260 | OH | TYR | A | 465 | −17.937 | −2.466 | 25.693 | 1.00 | 64.21 | A |
| ATOM | 3261 | C | TYR | A | 465 | −15.967 | 3.692 | 24.778 | 1.00 | 65.59 | A |
| ATOM | 3262 | O | TYR | A | 465 | −15.989 | 4.710 | 25.453 | 1.00 | 65.56 | A |
| ATOM | 3263 | N | ALA | A | 466 | −17.052 | 3.167 | 24.227 | 1.00 | 68.27 | A |
| ATOM | 3264 | CA | ALA | A | 466 | −18.362 | 3.756 | 24.430 | 1.00 | 70.93 | A |
| ATOM | 3265 | CB | ALA | A | 466 | −18.571 | 4.887 | 23.477 | 1.00 | 72.01 | A |
| ATOM | 3266 | C | ALA | A | 466 | −19.446 | 2.724 | 24.232 | 1.00 | 74.27 | A |
| ATOM | 3267 | O | ALA | A | 466 | −20.467 | 3.007 | 23.615 | 1.00 | 75.74 | A |
| ATOM | 3268 | N | GLY | A | 467 | −19.228 | 1.522 | 24.751 | 1.00 | 77.53 | A |
| ATOM | 3269 | CA | GLY | A | 467 | −20.229 | .480 | 24.609 | 1.00 | 80.89 | A |
| ATOM | 3270 | C | GLY | A | 467 | −21.470 | .782 | 25.437 | 1.00 | 82.73 | A |
| ATOM | 3271 | O | GLY | A | 467 | −21.931 | 1.932 | 25.514 | 1.00 | 81.82 | A |
| ATOM | 3272 | N | ASN | A | 468 | −22.027 | −.262 | 26.041 | 1.00 | 83.79 | A |
| ATOM | 3273 | CA | ASN | A | 468 | −23.205 | −.112 | 26.883 | 1.00 | 84.51 | A |
| ATOM | 3274 | CB | ASN | A | 468 | −24.470 | −.614 | 26.163 | 1.00 | 85.29 | A |
| ATOM | 3275 | CG | ASN | A | 468 | −24.590 | −2.123 | 26.159 | 1.00 | 86.98 | A |
| ATOM | 3276 | OD1 | ASN | A | 468 | −23.604 | −2.842 | 25.984 | 1.00 | 87.63 | A |
| ATOM | 3277 | ND2 | ASN | A | 468 | −25.814 | −2.614 | 26.337 | 1.00 | 87.84 | A |
| ATOM | 3278 | C | ASN | A | 468 | −22.923 | −.913 | 28.146 | 1.00 | 84.24 | A |
| ATOM | 3279 | O | ASN | A | 468 | −23.816 | −1.192 | 28.953 | 1.00 | 84.83 | A |
| ATOM | 3280 | N | HIS | A | 469 | −21.656 | −1.283 | 28.299 | 1.00 | 83.09 | A |
| ATOM | 3281 | CA | HIS | A | 469 | −21.212 | −2.010 | 29.472 | 1.00 | 81.98 | A |
| ATOM | 3282 | CB | HIS | A | 469 | −20.039 | −2.934 | 29.146 | 1.00 | 82.25 | A |
| ATOM | 3283 | CG | HIS | A | 469 | −20.363 | −4.001 | 28.147 | 1.00 | 83.39 | A |
| ATOM | 3284 | CD2 | HIS | A | 469 | −21.161 | −3.989 | 27.051 | 1.00 | 84.10 | A |
| ATOM | 3285 | ND1 | HIS | A | 469 | −19.814 | −5.264 | 28.207 | 1.00 | 81.93 | A |
| ATOM | 3286 | CE1 | HIS | A | 469 | −20.261 | −5.983 | 27.193 | 1.00 | 82.32 | A |
| ATOM | 3287 | NE2 | HIS | A | 469 | −21.080 | −5.234 | 26.476 | 1.00 | 83.45 | A |
| ATOM | 3288 | C | HIS | A | 469 | −20.753 | −.959 | 30.467 | 1.00 | 81.29 | A |
| ATOM | 3289 | O | HIS | A | 469 | −20.219 | −1.285 | 31.522 | 1.00 | 81.98 | A |
| ATOM | 3290 | N | ARG | A | 470 | −20.950 | .308 | 30.115 | 1.00 | 80.22 | A |
| ATOM | 3291 | CA | ARG | A | 470 | −20.556 | 1.396 | 30.996 | 1.00 | 79.96 | A |
| ATOM | 3292 | CB | ARG | A | 470 | −19.495 | 2.275 | 30.322 | 1.00 | 79.07 | A |
| ATOM | 3293 | CG | ARG | A | 470 | −18.689 | 3.140 | 31.294 | 1.00 | 76.67 | A |
| ATOM | 3294 | CD | ARG | A | 470 | −18.410 | 4.539 | 30.739 | 1.00 | 76.89 | A |
| ATOM | 3295 | NE | ARG | A | 470 | −17.445 | 4.569 | 29.635 | 1.00 | 76.11 | A |
| ATOM | 3296 | CZ | ARG | A | 470 | −16.127 | 4.445 | 29.774 | 1.00 | 75.32 | A |
| ATOM | 3297 | NH1 | ARG | A | 470 | −15.591 | 4.276 | 30.976 | 1.00 | 75.36 | A |
| ATOM | 3298 | NH2 | ARG | A | 470 | −15.339 | 4.506 | 28.710 | 1.00 | 73.44 | A |
| ATOM | 3299 | C | ARG | A | 470 | −21.767 | 2.251 | 31.394 | 1.00 | 80.60 | A |
| ATOM | 3300 | O | ARG | A | 470 | −22.344 | 2.075 | 32.473 | 1.00 | 81.10 | A |
| ATOM | 3301 | N | GLY | A | 471 | −22.159 | 3.167 | 30.516 | 1.00 | 80.13 | A |
| ATOM | 3302 | CA | GLY | A | 471 | −23.282 | 4.038 | 30.818 | 1.00 | 79.98 | A |
| ATOM | 3303 | C | GLY | A | 471 | −24.582 | 3.390 | 31.272 | 1.00 | 79.15 | A |
| ATOM | 3304 | O | GLY | A | 471 | −25.281 | 3.921 | 32.137 | 1.00 | 78.66 | A |
| ATOM | 3305 | N | GLY | A | 472 | −24.919 | 2.247 | 30.687 | 1.00 | 78.15 | A |
| ATOM | 3306 | CA | GLY | A | 472 | −26.157 | 1.585 | 31.047 | 1.00 | 77.47 | A |
| ATOM | 3307 | C | GLY | A | 472 | −26.783 | .938 | 29.830 | 1.00 | 76.71 | A |
| ATOM | 3308 | O | GLY | A | 472 | −26.477 | 1.306 | 28.698 | 1.00 | 77.67 | A |
| ATOM | 3309 | N | LEU | A | 473 | −27.670 | −.021 | 30.053 | 1.00 | 75.43 | A |
| ATOM | 3310 | CA | LEU | A | 473 | −28.298 | −.720 | 28.950 | 1.00 | 73.59 | A |
| ATOM | 3311 | CB | LEU | A | 473 | −28.344 | −2.226 | 29.245 | 1.00 | 73.79 | A |
| ATOM | 3312 | CG | LEU | A | 473 | −28.491 | −2.698 | 30.694 | 1.00 | 73.05 | A |
| ATOM | 3313 | CD1 | LEU | A | 473 | −29.673 | −2.035 | 31.367 | 1.00 | 72.63 | A |
| ATOM | 3314 | CD2 | LEU | A | 473 | −28.652 | −4.200 | 30.695 | 1.00 | 73.17 | A |
| ATOM | 3315 | C | LEU | A | 473 | −29.684 | −.210 | 28.584 | 1.00 | 72.13 | A |
| ATOM | 3316 | O | LEU | A | 473 | −30.685 | −.621 | 29.162 | 1.00 | 73.48 | A |
| ATOM | 3317 | N | SER | A | 474 | −29.717 | .690 | 27.606 | 1.00 | 69.39 | A |
| ATOM | 3318 | CA | SER | A | 474 | −30.948 | 1.284 | 27.088 | 1.00 | 64.95 | A |
| ATOM | 3319 | CB | SER | A | 474 | −31.617 | 2.170 | 28.134 | 1.00 | 62.53 | A |
| ATOM | 3320 | OG | SER | A | 474 | −30.965 | 3.424 | 28.222 | 1.00 | 58.92 | A |
| ATOM | 3321 | C | SER | A | 474 | −30.571 | 2.142 | 25.879 | 1.00 | 63.73 | A |
| ATOM | 3322 | O | SER | A | 474 | −29.583 | 2.881 | 25.913 | 1.00 | 63.08 | A |
| ATOM | 3323 | N | VAL | A | 475 | −31.352 | 2.039 | 24.812 | 1.00 | 61.00 | A |
| ATOM | 3324 | CA | VAL | A | 475 | −31.078 | 2.808 | 23.615 | 1.00 | 58.84 | A |
| ATOM | 3325 | CB | VAL | A | 475 | −32.319 | 2.932 | 22.758 | 1.00 | 56.99 | A |
| ATOM | 3326 | CG1 | VAL | A | 475 | −31.959 | 3.521 | 21.428 | 1.00 | 55.26 | A |
| ATOM | 3327 | CG2 | VAL | A | 475 | −32.951 | 1.579 | 22.595 | 1.00 | 57.79 | A |
| ATOM | 3328 | C | VAL | A | 475 | −30.606 | 4.206 | 23.977 | 1.00 | 58.60 | A |
| ATOM | 3329 | O | VAL | A | 475 | −29.523 | 4.631 | 23.587 | 1.00 | 58.04 | A |
| ATOM | 3330 | N | GLY | A | 476 | −31.426 | 4.912 | 24.742 | 1.00 | 58.69 | A |
| ATOM | 3331 | CA | GLY | A | 476 | −31.088 | 6.260 | 25.142 | 1.00 | 58.17 | A |
| ATOM | 3332 | C | GLY | A | 476 | −29.701 | 6.423 | 25.726 | 1.00 | 58.13 | A |
| ATOM | 3333 | O | GLY | A | 476 | −28.980 | 7.330 | 25.325 | 1.00 | 58.60 | A |
| ATOM | 3334 | N | LYS | A | 477 | −29.324 | 5.563 | 26.668 | 1.00 | 58.56 | A |
| ATOM | 3335 | CA | LYS | A | 477 | −28.005 | 5.655 | 27.307 | 1.00 | 59.49 | A |
| ATOM | 3336 | CB | LYS | A | 477 | −27.907 | 4.682 | 28.492 | 1.00 | 61.47 | A |
| ATOM | 3337 | CG | LYS | A | 477 | −28.617 | 5.119 | 29.775 | 1.00 | 64.03 | A |
| ATOM | 3338 | CD | LYS | A | 477 | −27.797 | 6.149 | 30.545 | 1.00 | 67.39 | A |
| ATOM | 3339 | CE | LYS | A | 477 | −28.466 | 6.564 | 31.857 | 1.00 | 68.34 | A |
| ATOM | 3340 | NZ | LYS | A | 477 | −29.741 | 7.330 | 31.672 | 1.00 | 67.87 | A |
| ATOM | 3341 | C | LYS | A | 477 | −26.839 | 5.400 | 26.346 | 1.00 | 58.10 | A |
| ATOM | 3342 | O | LYS | A | 477 | −25.863 | 6.150 | 26.353 | 1.00 | 58.13 | A |
| ATOM | 3343 | N | ALA | A | 478 | −26.932 | 4.346 | 25.537 | 1.00 | 55.53 | A |
| ATOM | 3344 | CA | ALA | A | 478 | −25.879 | 4.031 | 24.578 | 1.00 | 54.65 | A |
| ATOM | 3345 | CB | ALA | A | 478 | −26.231 | 2.778 | 23.796 | 1.00 | 54.51 | A |
| ATOM | 3346 | C | ALA | A | 478 | −25.708 | 5.205 | 23.626 | 1.00 | 54.59 | A |
| ATOM | 3347 | O | ALA | A | 478 | −24.597 | 5.620 | 23.313 | 1.00 | 54.62 | A |
| ATOM | 3348 | N | LEU | A | 479 | −26.828 | 5.740 | 23.167 | 1.00 | 55.22 | A |
| ATOM | 3349 | CA | LEU | A | 479 | −26.822 | 6.879 | 22.262 | 1.00 | 54.58 | A |
| ATOM | 3350 | CB | LEU | A | 479 | −28.251 | 7.391 | 22.084 | 1.00 | 52.73 | A |
| ATOM | 3351 | CG | LEU | A | 479 | −28.480 | 8.423 | 20.991 | 1.00 | 52.60 | A |
| ATOM | 3352 | CD1 | LEU | A | 479 | −29.928 | 8.361 | 20.528 | 1.00 | 54.85 | A |
| ATOM | 3353 | CD2 | LEU | A | 479 | −28.120 | 9.795 | 21.505 | 1.00 | 51.21 | A |
| ATOM | 3354 | C | LEU | A | 479 | −25.934 | 7.972 | 22.846 | 1.00 | 55.16 | A |
| ATOM | 3355 | O | LEU | A | 479 | −25.101 | 8.547 | 22.154 | 1.00 | 55.20 | A |
| ATOM | 3356 | N | SER | A | 480 | −26.116 | 8.242 | 24.131 | 1.00 | 56.48 | A |
| ATOM | 3357 | CA | SER | A | 480 | −25.343 | 9.258 | 24.826 | 1.00 | 57.99 | A |
| ATOM | 3358 | CB | SER | A | 480 | −25.931 | 9.508 | 26.209 | 1.00 | 57.90 | A |
| ATOM | 3359 | OG | SER | A | 480 | −27.151 | 10.216 | 26.117 | 1.00 | 63.19 | A |
| ATOM | 3360 | C | SER | A | 480 | −23.868 | 8.912 | 24.981 | 1.00. | 58.73 | A |
| ATOM | 3361 | O | SER | A | 480 | −23.015 | 9.795 | 24.930 | 1.00 | 59.91 | A |
| ATOM | 3362 | N | SER | A | 481 | −23.552 | 7.643 | 25.198 | 1.00 | 58.20 | A |
| ATOM | 3363 | CA | SER | A | 481 | −22.157 | 7.296 | 25.353 | 1.00 | 59.63 | A |
| ATOM | 3364 | CB | SER | A | 481 | −22.007 | 5.878 | 25.898 | 1.00 | 59.50 | A |
| ATOM | 3365 | OG | SER | A | 481 | −22.665 | 4.939 | 25.074 | 1.00 | 61.59 | A |
| ATOM | 3366 | C | SER | A | 481 | −21.486 | 7.434 | 23.994 | 1.00 | 61.18 | A |
| ATOM | 3367 | O | SER | A | 481 | −20.272 | 7.614 | 23.901 | 1.00 | 61.79 | A |
| ATOM | 3368 | N | GLY | A | 482 | −22.289 | 7.360 | 22.939 | 1.00 | 61.34 | A |
| ATOM | 3369 | CA | GLY | A | 482 | −21.756 | 7.508 | 21.601 | 1.00 | 61.28 | A |
| ATOM | 3370 | C | GLY | A | 482 | −21.401 | 8.965 | 21.352 | 1.00 | 61.89 | A |
| ATOM | 3371 | O | GLY | A | 482 | −20.305 | 9.280 | 20.881 | 1.00 | 62.12 | A |
| ATOM | 3372 | N | CYS | A | 483 | −22.331 | 9.861 | 21.667 | 1.00 | 61.64 | A |
| ATOM | 3373 | CA | CYS | A | 483 | −22.097 | 11.290 | 21.487 | 1.00 | 61.27 | A |
| ATOM | 3374 | CB | CYS | A | 483 | −23.311 | 12.105 | 21.910 | 1.00 | 59.45 | A |
| ATOM | 3375 | SG | CYS | A | 483 | −24.675 | 12.033 | 20.780 | 1.00 | 62.73 | A |
| ATOM | 3376 | C | CYS | A | 483 | −20.930 | 11.728 | 22.334 | 1.00 | 61.45 | A |
| ATOM | 3377 | O | CYS | A | 483 | −20.011 | 12.390 | 21.857 | 1.00 | 62.26 | A |
| ATOM | 3378 | N | ASN | A | 484 | −20.984 | 11.354 | 23.603 | 1.00 | 61.70 | A |
| ATOM | 3379 | CA | ASN | A | 484 | −19.959 | 11.718 | 24.551 | 1.00 | 61.61 | A |
| ATOM | 3380 | CB | ASN | A | 484 | −20.342 | 11.207 | 25.924 | 1.00 | 63.89 | A |
| ATOM | 3381 | CG | ASN | A | 484 | −19.659 | 11.970 | 27.015 | 1.00 | 68.57 | A |
| ATOM | 3382 | OD1 | ASN | A | 484 | −19.956 | 13.150 | 27.242 | 1.00 | 71.37 | A |
| ATOM | 3383 | ND2 | ASN | A | 484 | −18.719 | 11.317 | 27.697 | 1.00 | 70.18 | A |
| ATOM | 3384 | C | ASN | A | 484 | −18.587 | 11.185 | 24.161 | 1.00 | 61.15 | A |
| ATOM | 3385 | O | ASN | A | 484 | −17.564 | 11.695 | 24.595 | 1.00 | 59.17 | A |
| ATOM | 3386 | N | ALA | A | 485 | −18.568 | 10.152 | 23.333 | 1.00 | 62.18 | A |
| ATOM | 3387 | CA | ALA | A | 485 | −17.310 | 9.570 | 22.884 | 1.00 | 62.07 | A |
| ATOM | 3388 | CB | ALA | A | 485 | −17.527 | 8.132 | 22.427 | 1.00 | 62.05 | A |
| ATOM | 3389 | C | ALA | A | 485 | −16.774 | 10.397 | 21.732 | 1.00 | 61.68 | A |
| ATOM | 3390 | O | ALA | A | 485 | −15.571 | 10.591 | 21.593 | 1.00 | 61.18 | A |
| ATOM | 3391 | N | ALA | A | 486 | −17.685 | 10.880 | 20.900 | 1.00 | 61.93 | A |
| ATOM | 3392 | CA | ALA | A | 486 | −17.303 | 11.678 | 19.752 | 1.00 | 62.12 | A |
| ATOM | 3393 | CB | ALA | A | 486 | −18.519 | 11.962 | 18.870 | 1.00 | 61.93 | A |
| ATOM | 3394 | C | ALA | A | 486 | −16.633 | 12.979 | 20.148 | 1.00 | 62.37 | A |
| ATOM | 3395 | O | ALA | A | 486 | −15.577 | 13.314 | 19.624 | 1.00 | 63.63 | A |
| ATOM | 3396 | N | ASP | A | 487 | −17.216 | 13.732 | 21.067 | 1.00 | 62.50 | A |
| ATOM | 3397 | CA | ASP | A | 487 | −16.566 | 14.988 | 21.410 | 1.00 | 63.60 | A |
| ATOM | 3398 | CB | ASP | A | 487 | −17.570 | 16.004 | 21.951 | 1.00 | 65.85 | A |
| ATOM | 3399 | CG | ASP | A | 487 | −18.374 | 15.463 | 23.087 | 1.00 | 67.06 | A |
| ATOM | 3400 | OD1 | ASP | A | 487 | −17.843 | 14.595 | 23.816 | 1.00 | 68.67 | A |
| ATOM | 3401 | OD2 | ASP | A | 487 | −19.526 | 15.918 | 23.254 | 1.00 | 67.14 | A |
| ATOM | 3402 | C | ASP | A | 487 | −15.402 | 14.825 | 22.372 | 1.00 | 62.18 | A |
| ATOM | 3403 | O | ASP | A | 487 | −14.757 | 15.805 | 22.751 | 1.00 | 61.70 | A |
| ATOM | 3404 | N | LEU | A | 488 | −15.143 | 13.585 | 22.767 | 1.00 | 61.39 | A |
| ATOM | 3405 | CA | LEU | A | 488 | −14.015 | 13.284 | 23.642 | 1.00 | 60.67 | A |
| ATOM | 3406 | CB | LEU | A | 488 | −14.262 | 11.987 | 24.426 | 1.00 | 61.76 | A |
| ATOM | 3407 | CG | LEU | A | 488 | −14.067 | 11.994 | 25.952 | 1.00 | 60.58 | A |
| ATOM | 3408 | CD1 | LEU | A | 48.8 | −15.143 | 12.820 | 26.643 | 1.00 | 59.79 | A |
| ATOM | 3409 | CD2 | LEU | A | 488 | −14.126 | 10.568 | 26.453 | 1.00 | 60.21 | A |
| ATOM | 3410 | C | LEU | A | 488 | −12.871 | 13.101 | 22.643 | 1.00 | 59.88 | A |
| ATOM | 3411 | O | LEU | A | 488 | −11.747 | 13.541 | 22.868 | 1.00 | 58.40 | A |
| ATOM | 3412 | N | VAL | A | 489 | −13.187 | 12.452 | 21.528 | 1.00 | 59.87 | A |
| ATOM | 3413 | CA | VAL | A | 489 | −12.232 | 12.239 | 20.444 | 1.00 | 61.25 | A |
| ATOM | 3414 | CB | VAL | A | 489 | −12.873 | 11.451 | 19.265 | 1.00 | 61.82 | A |
| ATOM | 3415 | CG1 | VAL | A | 489 | −12.031 | 11.611 | 18.003 | 1.00 | 61.37 | A |
| ATOM | 3416 | CG2 | VAL | A | 489 | −13.026 | 9.993 | 19.627 | 1.00 | 62.78 | A |
| ATOM | 3417 | C | VAL | A | 489 | −11.847 | 13.615 | 19.911 | 1.00 | 61.87 | A |
| ATOM | 3418 | O | VAL | A | 489 | −10.683 | 13.883 | 19.614 | 1.00 | 61.75 | A |
| ATOM | 3419 | N | ILE | A | 490 | −12.855 | 14.478 | 19.788 | 1.00 | 62.35 | A |
| ATOM | 3420 | CA | ILE | A | 490 | −12.688 | 15.836 | 19.282 | 1.00 | 60.62 | A |
| ATOM | 3421 | CB | ILE | A | 490 | −14.071 | 16.540 | 19.162 | 1.00 | 60.63 | A |
| ATOM | 3422 | CG2 | ILE | A | 490 | −13.894 | 18.019 | 18.881 | 1.00 | 61.54 | A |
| ATOM | 3423 | CG1 | ILE | A | 490 | −14.888 | 15.889 | 18.041 | 1.00 | 59.62 | A |
| ATOM | 3424 | CD1 | ILE | A | 490 | −16.213 | 16.574 | 17.769 | 1.00 | 58.38 | A |
| ATOM | 3425 | C | ILE | A | 490 | −11.720 | 16.718 | 20.083 | 1.00 | 58.45 | A |
| ATOM | 3426 | O | ILE | A | 490 | −10.829 | 17.330 | 19.506 | 1.00 | 57.48 | A |
| ATOM | 3427 | N | SER | A | 491 | −11.867 | 16.790 | 21.399 | 1.00 | 55.86 | A |
| ATOM | 3428 | CA | SER | A | 491 | −10.963 | 17.641 | 22.157 | 1.00 | 55.85 | A |
| ATOM | 3429 | CB | SER | A | 491 | −11.572 | 18.024 | 23.507 | 1.00 | 54.64 | A |
| ATOM | 3.430 | OG | SER | A | 491 | −11.677 | 16.909 | 24.358 | 1.00 | 51.19 | A |
| ATOM | 3431 | C | SER | A | 491 | −9.609 | 16.988 | 22.373 | 1.00 | 57.33 | A |
| ATOM | 3432 | O | SER | A | 491 | −8.774 | 17.503 | 23.111 | 1.00 | 58.33 | A |
| ATOM | 3433 | N | TYR | A | 492 | −9.398 | 15.849 | 21.727 | 1.00 | 58.05 | A |
| ATOM | 3434 | CA | TYR | A | 492 | −8.145 | 15.115 | 21.840 | 1.00 | 57.42 | A |
| ATOM | 3435 | CB | TYR | A | 492 | −8.420 | 13.617 | 22.029 | 1.00 | 58.04 | A |
| ATOM | 3436 | CG | TYR | A | 492 | −7.363 | 12.690 | 21.456 | 1.00 | 57.71 | A |
| ATOM | 3437 | CD1 | TYR | A | 492 | −6.159 | 12.452 | 22.124 | 1.00 | 57.09 | A |
| ATOM | 3438 | CE1 | TYR | A | 492 | −5.191 | 11.605 | 21.587 | 1.00 | 56.52 | A |
| ATOM | 3439 | CD2 | TYR | A | 492 | −7.571 | 12.060 | 20.238 | 1.00 | 57.27 | A |
| ATOM | 3440 | CE2 | TYR | A | 492 | −6.620 | 11.222 | 19.697 | 1.00 | 58.68 | A |
| ATOM | 3441 | CZ | TYR | A | 492 | −5.433 | 10.994 | 20.371 | 1.00 | 58.86 | A |
| ATOM | 3442 | OH | TYR | A | 492 | −4.510 | 10.139 | 19.804 | 1.00 | 62.17 | A |
| ATOM | 3443 | C | TYR | A | 492 | −7.397 | 15.356 | 20.553 | 1.00 | 56.92 | A |
| ATOM | 3444 | O | TYR | A | 492 | −6.184 | 15.537 | 20.556 | 1.00 | 57.88 | A |
| ATOM | 3445 | N | LEU | A | 493 | −8.131 | 15.340 | 19.451 | 1.00 | 56.50 | A |
| ATOM | 3446 | CA | LEU | A | 493 | −7.542 | 15.598 | 18.154 | 1.00 | 57.85 | A |
| ATOM | 3447 | CB | LEU | A | 493 | −8.541 | 15.288 | 17.047 | 1.00 | 53.63 | A |
| ATOM | 3448 | CG | LEU | A | 493 | −6.808 | 13.837 | 16.703 | 1.00 | 51.77 | A |
| ATOM | 3449 | CD1 | LEU | A | 493 | −9.961 | 13.744 | 15.716 | 1.00 | 48.91 | A |
| ATOM | 3450 | CD2 | LEU | A | 493 | −7.551 | 13.234 | 16.124 | 1.00 | 50.06 | A |
| ATOM | 3451 | C | LEU | A | 493 | −7.146 | 17.080 | 18.067 | 1.00 | 61.21 | A |
| ATOM | 3452 | O | LEU | A | 493 | −6.269 | 17.445 | 17.280 | 1.00 | 62.10 | A |
| ATOM | 3453 | N | GLU | A | 494 | −7.795 | 17.921 | 18.879 | 1.00 | 64.13 | A |
| ATOM | 3454 | CA | GLU | A | 494 | −7.544 | 19.365 | 18.890 | 1.00 | 66.05 | A |
| ATOM | 3455 | CB | GLU | A | 494 | −8.857 | 20.138 | 18.956 | 1.00 | 65.54 | A |
| ATOM | 3456 | CG | GLU | A | 494 | −9.837 | 19.868 | 17.840 | 1.00 | 66.77 | A |
| ATOM | 3457 | CD | GLU | A | 494 | −11.130 | 20.648 | 18.025 | 1.00 | 67.51 | A |
| ATOM | 3458 | OE1 | GLU | A | 494 | −11.351 | 21.153 | 19.152 | 1.00 | 67.26 | A |
| ATOM | 3459 | OE2 | GLU | A | 494 | −11.926 | 20.746 | 17.057 | 1.00 | 67.38 | A |
| ATOM | 3460 | C | GLU | A | 494 | −6.683 | 19.838 | 20.051 | 1.00 | 68.91 | A |
| ATOM | 3461 | O | GLU | A | 494 | −6.365 | 21.021 | 20.143 | 1.00 | 69.98 | A |
| ATOM | 3462 | N | SER | A | 495 | −6.326 | 18.945 | 20.961 | 1.00 | 71.83 | A |
| ATOM | 3463 | CA | SER | A | 495 | −5.499 | 19.370 | 22.078 | 1.00 | 75.01 | A |
| ATOM | 3464 | CB | SER | A | 495 | −5.518 | 18.330 | 23.205 | 1.00 | 73.34 | A |
| ATOM | 3465 | OG | SER | A | 495 | −5.123 | 17.056 | 22.735 | 1.00 | 74.14 | A |
| ATOM | 3466 | C | SER | A | 495 | −4.088 | 19.557 | 21.554 | 1.00 | 77.38 | A |
| ATOM | 3467 | O | SER | A | 495 | −3.242 | 20.177 | 22.204 | 1.00 | 78.40 | A |
| ATOM | 3468 | N | VAL | A | 496 | −3.854 | 19.032 | 20.358 | 1.00 | 79.64 | A |
| ATOM | 3469 | CA | VAL | A | 496 | −2.549 | 19.108 | 19.724 | 1.00 | 84.10 | A |
| ATOM | 3470 | CB | VAL | A | 496 | −2.658 | 18.668 | 18.237 | 1.00 | 85.50 | A |
| ATOM | 3471 | CG1 | VAL | A | 496 | −3.094 | 17.192 | 18.169 | 1.00 | 85.80 | A |
| ATOM | 3472 | CG2 | VAL | A | 496 | −3.666 | 19.553 | 17.484 | 1.00 | 86.85 | A |
| ATOM | 3473 | C | VAL | A | 496 | −1.871 | 20.490 | 19.844 | 1.00 | 86.23 | A |
| ATOM | 3474 | O | VAL | A | 496 | −1.304 | 20.816 | 20.900 | 1.00 | 86.44 | A |
| ATOM | 3475 | N | SER | A | 497 | −1.919 | 21.284 | 18.769 | 1.00 | 88.07 | A |
| ATOM | 3476 | CA | SER | A | 497 | −1.322 | 22.629 | 18.730 | 1.00 | 89.18 | A |
| ATOM | 3477 | CB | SER | A | 497 | −1.717 | 23.430 | 19.987 | 1.00 | 88.98 | A |
| ATOM | 3478 | OG | SER | A | 497 | −3.127 | 23.562 | 20.113 | 1.00 | 88.34 | A |
| ATOM | 3479 | C | SER | A | 497 | .212 | 22.620 | 18.580 | 1.00 | 89.81 | A |
| ATOM | 3480 | O | SER | A | 497 | .703 | 23.154 | 17.556 | 1.00 | 89.42 | A |
| ATOM | 3481 | OXT | SER | A | 497 | .906 | 22.085 | 19.482 | 1.00 | 89.75 | A |
| ATOM | 3482 | CB | ALA | B | 13 | −74.110 | 58.322 | 36.617 | 1.00 | 100.00 | B |
| ATOM | 3483 | C | ALA | B | 13 | −73.160 | 56.222 | 37.631 | 1.00 | 100.00 | B |
| ATOM | 3484 | O | ALA | B | 13 | −74.104 | 56.252 | 38.429 | 1.00 | 99.52 | B |
| ATOM | 3485 | N | ALA | B | 13 | −72.473 | 56.978 | 35.335 | 1.00 | 100.00 | B |
| ATOM | 3486 | CA | ALA | B | 13 | −72.876 | 57.416 | 36.706 | 1.00 | 100.00 | B |
| ATOM | 3487 | N | LYS | B | 14 | −72.329 | 55.183 | 37.518 | 1.00 | 99.20 | B |
| ATOM | 3488 | CA | LYS | B | 14 | −72.458 | 53.965 | 38.329 | 1.00 | 97.76 | B |
| ATOM | 3489 | CB | LYS | B | 14 | −71.861 | 52.785 | 37.552 | 1.00 | 98.08 | B |
| ATOM | 3490 | CG | LYS | B | 14 | −72.218 | 51.402 | 38.080 | 1.00 | 98.96 | B |
| ATOM | 3491 | CD | LYS | B | 14 | −73.665 | 51.036 | 37.784 | 1.00 | 98.72 | B |
| ATOM | 3492 | CE | LYS | B | 14 | −73.911 | 49.533 | 37.976 | 1.00 | 99.50 | B |
| ATOM | 3493 | NZ | LYS | B | 14 | −73.680 | 49.040 | 39.369 | 1.00 | 99.47 | B |
| ATOM | 3494 | C | LYS | B | 14 | −71.755 | 54.126 | 39.701 | 1.00 | 96.10 | B |
| ATOM | 3495 | O | LYS | B | 14 | −70.523 | 54.168 | 39.789 | 1.00 | 95.62 | B |
| ATOM | 3496 | N | ARG | B | 15 | −72.552 | 54.207 | 40.765 | 1.00 | 93.72 | B |
| ATOM | 3497 | CA | ARG | B | 15 | −72.039 | 54.389 | 42.123 | 1.00 | 91.02 | B |
| ATOM | 3498 | CB | ARG | B | 15 | −73.040 | 55.226 | 42.938 | 1.00 | 93.42 | B |
| ATOM | 3499 | CG | ARG | B | 15 | −73.357 | 56.611 | 42.347 | 1.00 | 95.36 | B |
| ATOM | 3500 | CD | ARG | B | 15 | −72.692 | 57.769 | 43.118 | 1.00 | 96.45 | B |
| ATOM | 3501 | NE | ARG | B | 15 | −71.238 | 57.647 | 43.210 | 1.00 | 98.79 | B |
| ATOM | 3502 | CZ | ARG | B | 15 | −70.415 | 57.570 | 42.165 | 1.00 | 99.97 | B |
| ATOM | 3503 | NH1 | ARG | B | 15 | −70.897 | 57.602 | 40.926 | 1.00 | 100.00 | B |
| ATOM | 3504 | NH2 | ARG | B | 15 | −69.105 | 57.449 | 42.357 | 1.00 | 99.71 | B |
| ATOM | 3505 | C | ARG | B | 15 | −71.738 | 53.083 | 42.867 | 1.00 | 87.92 | B |
| ATOM | 3506 | O | ARG | B | 15 | −72.629 | 52.272 | 43.117 | 1.00 | 87.30 | B |
| ATOM | 3507 | N | VAL | B | 16 | −70.474 | 52.895 | 43.227 | 1.00 | 84.42 | B |
| ATOM | 3508 | CA | VAL | B | 16 | −70.044 | 51.699 | 43.946 | 1.00 | 81.21 | B |
| ATOM | 3509 | CB | VAL | B | 16 | −69.004 | 50.891 | 43.133 | 1.00 | 80.66 | B |
| ATOM | 3510 | CG1 | VAL | B | 16 | −68.715 | 49.568 | 43.824 | 1.00 | 79.05 | B |
| ATOM | 3511 | CG2 | VAL | B | 16 | −69.510 | 50.660 | 41.724 | 1.00 | 81.46 | B |
| ATOM | 3512 | C | VAL | B | 16 | −69.396 | 52.086 | 45.273 | 1.00 | 78.77 | B |
| ATOM | 3513 | O | VAL | B | 16 | −68.303 | 52.651 | 45.284 | 1.00 | 78.44 | B |
| ATOM | 3514 | N | ALA | B | 17 | −70.068 | 51.786 | 46.385 | 1.00 | 75.74 | B |
| ATOM | 3515 | CA | ALA | B | 17 | −69.536 | 52.095 | 47.715 | 1.00 | 71.93 | B |
| ATOM | 3516 | CB | ALA | B | 17 | −70.643 | 52.602 | 48.647 | 1.00 | 70.29 | B |
| ATOM | 3517 | C | ALA | B | 17 | −68.888 | 50.851 | 48.308 | 1.00 | 69.82 | B |
| ATOM | 3518 | O | ALA | B | 17 | −69.423 | 49.741 | 48.204 | 1.00 | 69.55 | B |
| ATOM | 3519 | N | VAL | B | 18 | −67.717 | 51.049 | 48.908 | 1.00 | 66.82 | B |
| ATOM | 3520 | CA | VAL | B | 18 | −66.966 | 49.975 | 49.537 | 1.00 | 63.07 | B |
| ATOM | 3521 | CB | VAL | B | 18 | −65.580 | 49.815 | 48.868 | 1.00 | 62.71 | B |
| ATOM | 3522 | CG1 | VAL | B | 18 | −64.923 | 48.521 | 49.306 | 1.00 | 63.34 | B |
| ATOM | 3523 | CG2 | VAL | B | 18 | −65.731 | 49.826 | 47.362 | 1.00 | 61.92 | B |
| ATOM | 3524 | C | VAL | B | 18 | −66.809 | 50.424 | 50.984 | 1.00 | 60.90 | B |
| ATOM | 3525 | O | VAL | B | 18 | −66.280 | 51.496 | 51.236 | 1.00 | 60.97 | B |
| ATOM | 3526 | N | ILE | B | 19 | −67.294 | 49.628 | 51.929 | 1.00 | 59.28 | B |
| ATOM | 3527 | CA | ILE | B | 19 | −67.199 | 49.992 | 53.340 | 1.00 | 59.18 | B |
| ATOM | 3528 | CB | ILE | B | 19 | −68.471 | 49.577 | 54.090 | 1.00 | 60.23 | B |
| ATOM | 3529 | CG2 | ILE | B | 19 | −68.313 | 49.818 | 55.587 | 1.00 | 58.99 | B |
| ATOM | 3530 | CG1 | ILE | B | 19 | −69.652 | 50.372 | 53.542 | 1.00 | 62.07 | B |
| ATOM | 3531 | CD1 | ILE | B | 19 | −70.956 | 50.087 | 54.262 | 1.00 | 65.56 | B |
| ATOM | 3532 | C | ILE | B | 19 | −65.969 | 49.411 | 54.053 | 1.00 | 57.98 | B |
| ATOM | 3533 | O | ILE | B | 19 | −65.906 | 48.217 | 54.369 | 1.00 | 57.11 | B |
| ATOM | 3534 | N | GLY | B | 20 | −65.000 | 50.282 | 54.320 | 1.00 | 55.75 | B |
| ATOM | 3535 | CA | GLY | B | 20 | −63.772 | 49.856 | 54.965 | 1.00 | 53.30 | B |
| ATOM | 3536 | C | GLY | B | 20 | −62.601 | 50.000 | 54.009 | 1.00 | 50.27 | B |
| ATOM | 3537 | O | GLY | B | 20 | −62.631 | 49.510 | 52.880 | 1.00 | 49.61 | B |
| ATOM | 3538 | N | ALA | B | 21 | −61.555 | 50.669 | 54.460 | 1.00 | 48.81 | B |
| ATOM | 3539 | CA | ALA | B | 21 | −60.400 | 50.873 | 53.603 | 1.00 | 48.41 | B |
| ATOM | 3540 | CB | ALA | B | 21 | −59.957 | 52.324 | 53.673 | 1.00 | 48.13 | B |
| ATOM | 3541 | C | ALA | B | 21 | −59.229 | 49.945 | 53.913 | 1.00 | 48.12 | B |
| ATOM | 3542 | O | ALA | B | 21 | −58.074 | 50.325 | 53.725 | 1.00 | 46.19 | B |
| ATOM | 3543 | N | GLY | B | 22 | −59.533 | 48.732 | 54.380 | 1.00 | 48.17 | B |
| ATOM | 3544 | CA | GLY | B | 22 | −58.483 | 47.768 | 54.673 | 1.00 | 48.02 | B |
| ATOM | 3545 | C | GLY | B | 22 | −57.880 | 47.265 | 53.371 | 1.00 | 48.01 | B |
| ATOM | 3546 | O | GLY | B | 22 | −58.263 | 47.733 | 52.296 | 1.00 | 48.13 | B |
| ATOM | 3547 | N | VAL | B | 23 | −56.942 | 46.326 | 53.432 | 1.00 | 47.49 | B |
| ATOM | 3548 | CA | VAL | B | 23 | −56.372 | 45.834 | 52.188 | 1.00 | 47.55 | B |
| ATOM | 3549 | CB | VAL | B | 23 | −55.383 | 44.707 | 52.403 | 1.00 | 45.23 | B |
| ATOM | 3550 | CG1 | VAL | B | 23 | −56.105 | 43.485 | 52.907 | 1.00 | 44.53 | B |
| ATOM | 3551 | CG2 | VAL | B | 23 | −54.673 | 44.411 | 51.098 | 1.00 | 43.79 | B |
| ATOM | 3552 | C | VAL | B | 23 | −57.495 | 45.308 | 51.297 | 1.00 | 48.58 | B |
| ATOM | 3553 | O | VAL | B | 23 | −57.499 | 45.554 | 50.091 | 1.00 | 49.40 | B |
| ATOM | 3554 | N | SER | B | 24 | −58.448 | 44.600 | 51.903 | 1.00 | 49.27 | B |
| ATOM | 3555 | CA | SER | B | 24 | −59.590 | 44.037 | 51.184 | 1.00 | 49.92 | B |
| ATOM | 3556 | CB | SER | B | 24 | −60.504 | 43.310 | 52.158 | 1.00 | 52.54 | B |
| ATOM | 3557 | OG | SER | B | 24 | −61.673 | 42.864 | 51.496 | 1.00 | 58.66 | B |
| ATOM | 3558 | C | SER | B | 24 | −60.404 | 45.093 | 50.442 | 1.00 | 49.01 | B |
| ATOM | 3559 | O | SER | B | 24 | −60.697 | 44.949 | 49.254 | 1.00 | 46.28 | B |
| ATOM | 3560 | N | GLY | B | 25 | −60.781 | 46.148 | 51.156 | 1.00 | 50.21 | B |
| ATOM | 3561 | CA | GLY | B | 25 | −61.543 | 47.226 | 50.539 | 1.00 | 52.06 | B |
| ATOM | 3562 | C | GLY | B | 25 | −60.771 | 47.923 | 49.424 | 1.00 | 52.59 | B |
| ATOM | 3563 | O | GLY | B | 25 | −61.302 | 48.148 | 48.331 | 1.00 | 49.78 | B |
| ATOM | 3564 | N | LEU | B | 26 | −59.509 | 48.254 | 49.710 | 1.00 | 53.60 | B |
| ATOM | 3565 | CA | LEU | B | 26 | −58.629 | 48.920 | 48.756 | 1.00 | 54.09 | B |
| ATOM | 3566 | CB | LEU | B | 26 | −57.317 | 49.290 | 49.440 | 1.00 | 54.00 | B |
| ATOM | 3567 | CG | LEU | B | 26 | −57.557 | 50.321 | 50.559 | 1.00 | 55.83 | B |
| ATOM | 3568 | CD1 | LEU | B | 26 | −56.293 | 50.581 | 51.356 | 1.00 | 55.88 | B |
| ATOM | 3569 | CD2 | LEU | B | 26 | −58.062 | 51.618 | 49.948 | 1.00 | 55.96 | B |
| ATOM | 3570 | C | LEU | B | 26 | −58.375 | 48.059 | 47.524 | 1.00 | 54.61 | B |
| ATOM | 3571 | O | LEU | B | 26 | −58.223 | 48.568 | 46.404 | 1.00 | 55.03 | B |
| ATOM | 3572 | N | ALA | B | 27 | −58.336 | 46.752 | 47.726 | 1.00 | 54.37 | B |
| ATOM | 3573 | CA | ALA | B | 27 | −58.144 | 45.842 | 46.610 | 1.00 | 55.71 | B |
| ATOM | 3574 | CB | ALA | B | 27 | −57.955 | 44.413 | 47.124 | 1.00 | 55.61 | B |
| ATOM | 3575 | C | ALA | B | 27 | −59.400 | 45.931 | 45.744 | 1.00 | 56.60 | B |
| ATOM | 3576 | O | ALA | B | 27 | −59.327 | 46.079 | 44.525 | 1.00 | 56.36 | B |
| ATOM | 3577 | N | ALA | B | 28 | −60.555 | 45.857 | 46.397 | 1.00 | 57.69 | B |
| ATOM | 3578 | CA | ALA | B | 28 | −61.832 | 45.914 | 45.707 | 1.00 | 58.86 | B |
| ATOM | 3579 | CB | ALA | B | 28 | −62.957 | 45.673 | 46.702 | 1.00 | 58.40 | B |
| ATOM | 3580 | C | ALA | B | 28 | −62.043 | 47.245 | 44.980 | 1.00 | 60.13 | B |
| ATOM | 3581 | O | ALA | B | 28 | −62.480 | 47.266 | 43.832 | 1.00 | 60.18 | B |
| ATOM | 3582 | N | ALA | B | 29 | −61.733 | 48.351 | 45.655 | 1.00 | 61.72 | B |
| ATOM | 3583 | CA | ALA | B | 29 | −61.901 | 49.680 | 45.076 | 1.00 | 62.27 | B |
| ATOM | 3584 | CB | ALA | B | 29 | −61.694 | 50.737 | 46.138 | 1.00 | 60.45 | B |
| ATOM | 3585 | C | ALA | B | 29 | −60.933 | 49.900 | 43.920 | 1.00 | 64.72 | B |
| ATOM | 3586 | O | ALA | B | 29 | −61.222 | 50.656 | 42.994 | 1.00 | 65.52 | B |
| ATOM | 3587 | N | TYR | B | 30 | −59.779 | 49.247 | 43.967 | 1.00 | 66.69 | B |
| ATOM | 3588 | CA | TYR | B | 30 | −58.810 | 49.386 | 42.886 | 1.00 | 69.38 | B |
| ATOM | 3589 | CB | TYR | B | 30 | −57.448 | 48.876 | 43.330 | 1.00 | 69.38 | B |
| ATOM | 3590 | CG | TYR | B | 30 | −56.350 | 49.122 | 42.327 | 1.00 | 69.27 | B |
| ATOM | 3591 | CD1 | TYR | B | 30 | −55.774 | 50.386 | 42.190 | 1.00 | 69.30 | B |
| ATOM | 3592 | CE1 | TYR | B | 30 | −54.729 | 50.605 | 41.304 | 1.00 | 71.31 | B |
| ATOM | 3593 | CD2 | TYR | B | 30 | −55.861 | 48.083 | 41.541 | 1.00 | 69.65 | B |
| ATOM | 3594 | CE2 | TYR | B | 30 | −54.819 | 48.285 | 40.650 | 1.00 | 71.35 | B |
| ATOM | 3595 | CZ | TYR | B | 30 | −54.253 | 49.546 | 40.537 | 1.00 | 73.06 | B |
| ATOM | 3596 | OH | TYR | B | 30 | −53.191 | 49.735 | 39.674 | 1.00 | 75.99 | B |
| ATOM | 3597 | C | TYR | B | 30 | −59.260 | 48.594 | 41.652 | 1.00 | 70.52 | B |
| ATOM | 3598 | O | TYR | B | 30 | −59.313 | 49.130 | 40.541 | 1.00 | 71.06 | B |
| ATOM | 3599 | N | LYS | B | 31 | −59.576 | 47.317 | 41.855 | 1.00 | 71.32 | B |
| ATOM | 3600 | CA | LYS | B | 31 | −60.022 | 46.450 | 40.767 | 1.00 | 72.74 | B |
| ATOM | 3601 | CB | LYS | B | 31 | −60.478 | 45.099 | 41.332 | 1.00 | 74.03 | B |
| ATOM | 3602 | CG | LYS | B | 31 | −60.307 | 43.910 | 40.388 | 1.00 | 75.89 | B |
| ATOM | 3603 | CD | LYS | B | 31 | −58.838 | 43.572 | 40.205 | 1.00 | 78.17 | B |
| ATOM | 3604 | CE | LYS | B | 31 | −58.657 | 42.266 | 39.444 | 1.00 | 79.82 | B |
| ATOM | 3605 | NZ | LYS | B | 31 | −57.223 | 41.832 | 39.416 | 1.00 | 80.60 | B |
| ATOM | 3606 | C | LYS | B | 31 | −61.187 | 47.132 | 40.037 | 1.00 | 73.05 | B |
| ATOM | 3607 | O | LYS | B | 31 | −61.297 | 47.067 | 38.803 | 1.00 | 74.03 | B |
| ATOM | 3608 | N | LEU | B | 32 | −62.047 | 47.789 | 40.812 | 1.00 | 71.64 | B |
| ATOM | 3609 | CA | LEU | B | 32 | −63.196 | 48.497 | 40.270 | 1.00 | 70.76 | B |
| ATOM | 3610 | CB | LEU | B | 32 | −64.052 | 49.052 | 41.404 | 1.00 | 69.09 | B |
| ATOM | 3611 | CG | LEU | B | 32 | −64.878 | 48.013 | 42.152 | 1.00 | 69.56 | B |
| ATOM | 3612 | CD1 | LEU | B | 32 | −65.417 | 48.604 | 43.439 | 1.00 | 70.14 | B |
| ATOM | 3613 | CD2 | LEU | B | 32 | −66.008 | 47.541 | 41.260 | 1.00 | 69.49 | B |
| ATOM | 3614 | C | LEU | B | 32 | −62.766 | 49.643 | 39.373 | 1.00 | 71.51 | B |
| ATOM | 3615 | O | LEU | B | 32 | −63.078 | 49.673 | 38.190 | 1.00 | 71.71 | B |
| ATOM | 3616 | N | LYS | B | 33 | −62.037 | 50.584 | 39.952 | 1.00 | 73.03 | B |
| ATOM | 3617 | CA | LYS | B | 33 | −61.570 | 51.764 | 39.235 | 1.00 | 74.08 | B |
| ATOM | 3618 | CB | LYS | B | 33 | −60.606 | 52.552 | 40.136 | 1.00 | 73.60 | B |
| ATOM | 3619 | CG | LYS | B | 33 | −60.207 | 53.908 | 39.610 | 1.00 | 72.97 | B |
| ATOM | 3620 | CD | LYS | B | 33 | −59.407 | 54.655 | 40.651 | 1.00 | 74.36 | B |
| ATOM | 3621 | CE | LYS | B | 33 | −58.727 | 55.888 | 40.068 | 1.00 | 76.05 | B |
| ATOM | 3622 | NZ | LYS | B | 33 | −59.695 | 56.936 | 39.648 | 1.00 | 76.45 | B |
| ATOM | 3623 | C | LYS | B | 33 | −60.927 | 51.496 | 37.866 | 1.00 | 74.16 | B |
| ATOM | 3624 | O | LYS | B | 33 | −61.220 | 52.202 | 36.895 | 1.00 | 74.64 | B |
| ATOM | 3625 | N | ILE | B | 34 | −60.067 | 50.485 | 37.769 | 1.00 | 73.51 | B |
| ATOM | 3626 | CA | ILE | B | 34 | −59.419 | 50.209 | 36.487 | 1.00 | 73.71 | B |
| ATOM | 3627 | CB | ILE | B | 34 | −58.161 | 49.323 | 36.643 | 1.00 | 73.34 | B |
| ATOM | 3628 | CG2 | ILE | B | 34 | −57.263 | 49.897 | 37.741 | 1.00 | 72.70 | B |
| ATOM | 3629 | CG1 | ILE | B | 34 | −58.565 | 47.872 | 36.931 | 1.00 | 72.54 | B |
| ATOM | 3630 | CD1 | ILE | B | 34 | −57.393 | 46.919 | 36.959 | 1.00 | 71.28 | B |
| ATOM | 3631 | C | ILE | B | 34 | −60.360 | 49.539 | 35.488 | 1.00 | 74.06 | B |
| ATOM | 3632 | O | ILE | B | 34 | −59.916 | 48.984 | 34.483 | 1.00 | 74.59 | B |
| ATOM | 3633 | N | HIS | B | 35 | −61.655 | 49.580 | 35.784 | 1.00 | 73.64 | B |
| ATOM | 3634 | CA | HIS | B | 35 | −62.682 | 49.024 | 34.908 | 1.00 | 73.29 | B |
| ATOM | 3635 | CB | HIS | B | 35 | −63.180 | 47.673 | 35.438 | 1.00 | 74.96 | B |
| ATOM | 3636 | CG | HIS | B | 35 | −64.253 | 47.047 | 34.596 | 1.00 | 76.52 | B |
| ATOM | 3637 | CD2 | HIS | B | 35 | −64.211 | 46.012 | 33.723 | 1.00 | 76.91 | B |
| ATOM | 3638 | ND1 | HIS | B | 35 | −65.554 | 47.509 | 34.576 | 1.00 | 76.72 | B |
| ATOM | 3639 | CE1 | HIS | B | 35 | −66.266 | 46.787 | 33.727 | 1.00 | 76.63 | B |
| ATOM | 3640 | NE2 | HIS | B | 35 | −65.474 | 45.872 | 33.196 | 1.00 | 77.33 | B |
| ATOM | 3641 | C | HIS | B | 35 | −63.813 | 50.058 | 34.877 | 1.00 | 73.54 | B |
| ATOM | 3642 | O | HIS | B | 35 | −65.005 | 49.736 | 34.933 | 1.00 | 71.65 | B |
| ATOM | 3643 | N | GLY | B | 36 | −63.410 | 51.323 | 34.815 | 1.00 | 73.42 | B |
| ATOM | 3644 | CA | GLY | B | 36 | −64.375 | 52.399 | 34.761 | 1.00 | 72.44 | B |
| ATOM | 3645 | C | GLY | B | 36 | −65.015 | 52.736 | 36.086 | 1.00 | 72.04 | B |
| ATOM | 3646 | O | GLY | B | 36 | −64.568 | 53.646 | 36.773 | 1.00 | 71.86 | B |
| ATOM | 3647 | N | LEU | B | 37 | −66.059 | 51.996 | 36.441 | 1.00 | 72.45 | B |
| ATOM | 3648 | CA | LEU | B | 37 | −66.816 | 52.217 | 37.675 | 1.00 | 73.11 | B |
| ATOM | 3649 | CB | LEU | B | 37 | −67.053 | 50.876 | 38.383 | 1.00 | 74.40 | B |
| ATOM | 3650 | CG | LEU | B | 37 | −67.791 | 49.813 | 37.556 | 1.00 | 75.61 | B |
| ATOM | 3651 | CD1 | LEU | B | 37 | −68.156 | 48.626 | 38.447 | 1.00 | 75.31 | B |
| ATOM | 3652 | CD2 | LEU | B | 37 | −69.057 | 50.414 | 36.942 | 1.00 | 75.62 | B |
| ATOM | 3653 | C | LEU | B | 37 | −66.264 | 53.240 | 38.684 | 1.00 | 72.41 | B |
| ATOM | 3654 | O | LEU | B | 37 | −65.162 | 53.080 | 39.231 | 1.00 | 71.88 | B |
| ATOM | 3655 | N | ASN | B | 38 | −67.047 | 54.292 | 38.915 | 1.00 | 71.05 | B |
| ATOM | 3656 | CA | ASN | B | 38 | −66.681 | 55.333 | 39.866 | 1.00 | 70.10 | B |
| ATOM | 3657 | CB | ASN | B | 38 | −67.618 | 56.532 | 39.705 | 1.00 | 69.33 | B |
| ATOM | 3658 | CG | ASN | B | 38 | −67.678 | 57.038 | 38.277 | .00 | 69.47 | B |
| ATOM | 3659 | OD1 | ASN | B | 38 | −66.695 | 57.556 | 37.747 | .00 | 69.41 | B |
| ATOM | 3660 | ND2 | ASN | B | 38 | −68.835 | 56.883 | 37.644 | .00 | 69.33 | B |
| ATOM | 3661 | C | ASN | B | 38 | −66.845 | 54.705 | 41.258 | 1.00 | 69.74 | B |
| ATOM | 3662 | O | ASN | B | 38 | −67.794 | 53.946 | 41.485 | 1.00 | 69.83 | B |
| ATOM | 3663 | N | VAL | B | 39 | −65.931 | 55.008 | 42.181 | 1.00 | 68.27 | B |
| ATOM | 3664 | CA | VAL | B | 39 | −66.002 | 54.429 | 43.518 | 1.00 | 66.60 | B |
| ATOM | 3665 | CB | VAL | B | 39 | −64.898 | 53.361 | 43.720 | 1.00 | 66.44 | B |
| ATOM | 3666 | CG1 | VAL | B | 39 | −65.074 | 52.672 | 45.067 | 1.00 | 67.38 | B |
| ATOM | 3667 | CG2 | VAL | B | 39 | −64.945 | 52.337 | 42.609 | 1.00 | 66.14 | B |
| ATOM | 3668 | C | VAL | B | 39 | −65.876 | 55.438 | 44.651 | 1.00 | 66.06 | B |
| ATOM | 3669 | O | VAL | B | 39 | −65.348 | 56.532 | 44.470 | 1.00 | 66.35 | B |
| ATOM | 3670 | N | THR | B | 40 | −66.377 | 55.042 | 45.819 | 1.00 | 65.25 | B |
| ATOM | 3671 | CA | THR | B | 40 | −66.318 | 55.834 | 47.039 | 1.00 | 65.10 | B |
| ATOM | 3672 | CB | THR | B | 40 | −67.658 | 56.535 | 47.337 | 1.00 | 65.22 | B |
| ATOM | 3673 | OG1 | THR | B | 40 | −67.875 | 57.578 | 46.380 | 1.00 | 68.13 | B |
| ATOM | 3674 | CG2 | THR | B | 40 | −67.644 | 57.152 | 48.715 | 1.00 | 66.12 | B |
| ATOM | 3675 | C | THR | B | 40 | −66.001 | 54.846 | 48.161 | 1.00 | 65.26 | B |
| ATOM | 3676 | O | THR | B | 40 | −66.619 | 53.791 | 48.257 | 1.00 | 66.47 | B |
| ATOM | 3677 | N | VAL | B | 41 | −65.028 | 55.184 | 49.001 | 1.00 | 64.67 | B |
| ATOM | 3678 | CA | VAL | B | 41 | −64.624 | 54.313 | 50.097 | 1.00 | 63.18 | B |
| ATOM | 3679 | CB | VAL | B | 41 | −63.150 | 53.885 | 49.936 | 1.00 | 62.22 | B |
| ATOM | 3680 | CG1 | VAL | B | 41 | −62.755 | 52.953 | 51.057 | 1.00 | 61.36 | B |
| ATOM | 3681 | CG2 | VAL | B | 41 | −62.937 | 53.227 | 48.593 | 1.00 | 61.33 | B |
| ATOM | 3682 | C | VAL | B | 41 | −64.778 | 54.969 | 51.470 | 1.00 | 64.04 | B |
| ATOM | 3683 | O | VAL | B | 41 | −64.123 | 55.971 | 51.768 | 1.00 | 63.72 | B |
| ATOM | 3684 | N | PHE | B | 42 | −65.640 | 54.403 | 52.306 | 1.00 | 64.59 | B |
| ATOM | 3685 | CA | PHE | B | 42 | −65.846 | 54.932 | 53.649 | 1.00 | 65.35 | B |
| ATOM | 3686 | CB | PHE | B | 42 | −67.299 | 54.739 | 54.081 | 1.00 | 63.93 | B |
| ATOM | 3687 | CG | PHE | B | 42 | −68.280 | 55.412 | 53.172 | 1.00 | 64.41 | B |
| ATOM | 3688 | CD1 | PHE | B | 42 | −68.541 | 54.897 | 51.902 | 1.00 | 62.98 | B |
| ATOM | 3689 | CD2 | PHE | B | 42 | −68.914 | 56.593 | 53.564 | 1.00 | 63.68 | B |
| ATOM | 3690 | CE1 | PHE | B | 42 | −69.412 | 55.544 | 51.038 | 1.00 | 61.93 | B |
| ATOM | 3691 | CE2 | PHE | B | 42 | −69.786 | 57.247 | 52.706 | 1.00 | 62.25 | B |
| ATOM | 3692 | CZ | PHE | B | 42 | −70.035 | 56.720 | 51.437 | 1.00 | 61.72 | B |
| ATOM | 3693 | C | PHE | B | 42 | −64.907 | 54.214 | 54.608 | 1.00 | 66.88 | B |
| ATOM | 3694 | O | PHE | B | 42 | −64.868 | 52.984 | 54.655 | 1.00 | 67.95 | B |
| ATOM | 3695 | N | GLU | B | 43 | −64.146 | 54.992 | 55.369 | 1.00 | 68.46 | B |
| ATOM | 3696 | CA | GLU | B | 43 | −63.181 | 54.446 | 56.315 | 1.00 | 69.25 | B |
| ATOM | 3697 | CB | GLU | B | 43 | −61.762 | 54.684 | 55.773 | 1.00 | 69.60 | B |
| ATOM | 3698 | CG | GLU | B | 43 | −60.634 | 54.551 | 56.783 | 1.00 | 70.27 | B |
| ATOM | 3699 | CD | GLU | B | 43 | −60.741 | 53.303 | 57.618 | 1.00 | 70.61 | B |
| ATOM | 3700 | OE1 | GLU | B | 43 | −61.452 | 53.340 | 58.638 | 1.00 | 70.43 | B |
| ATOM | 3701 | OE2 | GLU | B | 43 | −60.124 | 52.285 | 57.250 | 1.00 | 71.29 | B |
| ATOM | 3702 | C | GLU | B | 43 | −63.363 | 55.071 | 57.695 | 1.00 | 69.22 | B |
| ATOM | 3703 | O | GLU | B | 43 | −63.002 | 56.214 | 57.921 | 1.00 | 69.47 | B |
| ATOM | 3704 | N | ALA | B | 44 | −63.926 | 54.301 | 58.616 | 1.00 | 69.46 | B |
| ATOM | 3705 | CA | ALA | B | 44 | −64.187 | 54.766 | 59.971 | 1.00 | 70.00 | B |
| ATOM | 3706 | CB | ALA | B | 44 | −64.718 | 53.597 | 60.824 | 1.00 | 70.92 | B |
| ATOM | 3707 | C | ALA | B | 44 | −63.000 | 55.421 | 60.675 | 1.00 | 69.97 | B |
| ATOM | 3708 | O | ALA | B | 44 | −63.171 | 56.398 | 61.409 | 1.00 | 70.33 | B |
| ATOM | 3709 | N | GLU | B | 45 | −61.801 | 54.888 | 60.457 | 1.00 | 70.24 | B |
| ATOM | 3710 | CA | GLU | B | 45 | −60.593 | 55.410 | 61.108 | 1.00 | 70.06 | B |
| ATOM | 3711 | CB | GLU | B | 45 | −59.558 | 54.286 | 61.244 | 1.00 | 71.23 | B |
| ATOM | 3712 | CG | GLU | B | 45 | −59.039 | 54.047 | 62.667 | 1.00 | 74.57 | B |
| ATOM | 3713 | CD | GLU | B | 45 | −60.115 | 53.571 | 63.653 | 1.00 | 77.70 | B |
| ATOM | 3714 | OE1 | GLU | B | 45 | −59.754 | 52.825 | 64.597 | 1.00 | 78.58 | B |
| ATOM | 3715 | OE2 | GLU | B | 45 | −61.308 | 53.944 | 63.502 | 1.00 | 78.90 | B |
| ATOM | 3716 | C | GLU | B | 45 | −59.957 | 56.618 | 60.406 | 1.00 | 68.47 | B |
| ATOM | 3717 | O | GLU | B | 45 | −60.269 | 56.927 | 59.260 | 1.00 | 67.69 | B |
| ATOM | 3718 | N | GLY | B | 46 | −59.056 | 57.293 | 61.111 | 1.00 | 66.97 | B |
| ATOM | 3719 | CA | GLY | B | 46 | −58.390 | 58.457 | 60.552 | 1.00 | 66.52 | B |
| ATOM | 3720 | C | GLY | B | 46 | −57.421 | 58.201 | 59.403 | 1.00 | 66.86 | B |
| ATOM | 3721 | O | GLY | B | 46 | −57.235 | 59.062 | 58.547 | 1.00 | 66.53 | B |
| ATOM | 3722 | N | LYS | B | 47 | −56.775 | 57.039 | 59.385 | 1.00 | 67.03 | B |
| ATOM | 3723 | CA | LYS | B | 47 | −55.847 | 56.715 | 58.306 | 1.00 | 66.29 | B |
| ATOM | 3724 | CB | LYS | B | 47 | −54.417 | 56.512 | 58.852 | 1.00 | 67.23 | B |
| ATOM | 3725 | CG | LYS | B | 47 | −54.314 | 55.832 | 60.216 | 1.00 | 68.48 | B |
| ATOM | 3726 | CD | LYS | B | 47 | −52.879 | 55.874 | 60.790 | 1.00 | 71.30 | B |
| ATOM | 3727 | CE | LYS | B | 47 | −51.856 | 55.094 | 59.927 | 1.00 | 72.73 | B |
| ATOM | 3728 | NZ | LYS | B | 47 | −50.496 | 54.927 | 60.557 | 1.00 | 69.65 | B |
| ATOM | 3729 | C | LYS | B | 47 | −56.335 | 55.486 | 57.532 | 1.00 | 65.62 | B |
| ATOM | 3730 | O | LYS | B | 47 | −56.954 | 54.574 | 58.093 | 1.00 | 64.96 | B |
| ATOM | 3731 | N | ALA | B | 48 | −56.068 | 55.480 | 56.231 | 1.00 | 64.62 | B |
| ATOM | 3732 | CA | ALA | B | 48 | −56.504 | 54.390 | 55.369 | 1.00 | 63.91 | B |
| ATOM | 3733 | CB | ALA | B | 48 | −56.011 | 54.616 | 53.939 | 1.00 | 62.58 | B |
| ATOM | 3734 | C | ALA | B | 48 | −56.098 | 53.007 | 55.858 | 1.00 | 63.34 | B |
| ATOM | 3735 | O | ALA | B | 48 | −56.968 | 52.183 | 56.128 | 1.00 | 64.36 | B |
| ATOM | 3736 | N | GLY | B | 49 | −54.798 | 52.748 | 55.971 | 1.00 | 61.77 | B |
| ATOM | 3737 | CA | GLY | B | 49 | −54.342 | 51.443 | 56.427 | 1.00 | 62.32 | B |
| ATOM | 3738 | C | GLY | B | 49 | −55.385 | 50.546 | 57.111 | 1.00 | 61.71 | B |
| ATOM | 3739 | O | GLY | B | 49 | −56.129 | 50.994 | 57.988 | 1.00 | 63.01 | B |
| ATOM | 3740 | N | GLY | B | 50 | −55.458 | 49.278 | 56.725 | 1.00 | 59.07 | B |
| ATOM | 3741 | CA | GLY | B | 50 | −56.422 | 48.408 | 57.370 | 1.00 | 59.05 | B |
| ATOM | 3742 | C | GLY | B | 50 | −55.764 | 47.851 | 58.611 | 1.00 | 58.90 | B |
| ATOM | 3743 | O | GLY | B | 50 | −55.427 | 48.593 | 59.537 | 1.00 | 58.99 | B |
| ATOM | 3744 | N | LYS | B | 51 | −55.594 | 46.537 | 58.650 | 1.00 | 58.20 | B |
| ATOM | 3745 | CA | LYS | B | 51 | −54.902 | 45.930 | 59.769 | 1.00 | 56.58 | B |
| ATOM | 3746 | CB | LYS | B | 51 | −55.210 | 44.435 | 59.879 | 1.00 | 57.45 | B |
| ATOM | 3747 | CG | LYS | B | 51 | −56.532 | 44.100 | 60.534 | 1.00 | 56.85 | B |
| ATOM | 3748 | CD | LYS | B | 51 | −56.581 | 44.615 | 61.963 | 1.00 | 58.15 | B |
| ATOM | 3749 | CE | LYS | B | 51 | −57.780 | 44.044 | 62.716 | 1.00 | 58.85 | B |
| ATOM | 3750 | NZ | LYS | B | 51 | −59.070 | 44.330 | 62.024 | 1.00 | 62.31 | B |
| ATOM | 3751 | C | LYS | B | 51 | −53.438 | 46.140 | 59.408 | 1.00 | 54.94 | B |
| ATOM | 3752 | O | LYS | B | 51 | −52.559 | 45.779 | 60.171 | 1.00 | 55.15 | B |
| ATOM | 3753 | N | LEU | B | 52 | −53.194 | 46.693 | 58.215 | 1.00 | 53.00 | B |
| ATOM | 3754 | CA | LEU | B | 52 | −51.842 | 47.010 | 57.778 | 1.00 | 52.26 | B |
| ATOM | 3755 | CB | LEU | B | 52 | −51.748 | 47.056 | 56.248 | 1.00 | 51.51 | B |
| ATOM | 3756 | CG | LEU | B | 52 | −51.973 | 45.756 | 55.447 | 1.00 | 53.18 | B |
| ATOM | 3757 | CD1 | LEU | B | 52 | −51.385 | 45.915 | 54.036 | 1.00 | 51.70 | B |
| ATOM | 3758 | CD2 | LEU | B | 52 | −51.314 | 44.578 | 56.118 | 1.00 | 52.38 | B |
| ATOM | 3759 | C | LEU | B | 52 | −51.569 | 48.392 | 58.394 | 1.00 | 51.79 | B |
| ATOM | 3760 | O | LEU | B | 52 | −52.256 | 49.360 | 58.090 | 1.00 | 50.93 | B |
| ATOM | 3761 | N | ARG | B | 53 | −50.566 | 48.476 | 59.267 | 1.00 | 52.07 | B |
| ATOM | 3762 | CA | ARG | B | 53 | −50.272 | 49.714 | 59.971 | 1.00 | 51.52 | B |
| ATOM | 3763 | CB | ARG | B | 53 | −51.054 | 49.691 | 61.281 | 1.00 | 50.85 | B |
| ATOM | 3764 | CG | ARG | B | 53 | −51.288 | 51.011 | 61.942 | 1.00 | 52.98 | B |
| ATOM | 3765 | CD | ARG | B | 53 | −51.853 | 50.776 | 63.334 | 1.00 | 56.02 | B |
| ATOM | 3766 | NE | ARG | B | 53 | −52.292 | 52.012 | 63.981 | 1.00 | 58.88 | B |
| ATOM | 3767 | CZ | ARG | B | 53 | −53.416 | 52.652 | 63.679 | 1.00 | 58.35 | B |
| ATOM | 3768 | NH1 | ARG | B | 53 | −54.220 | 52.160 | 62.737 | 1.00 | 59.70 | B |
| ATOM | 3769 | NH2 | ARG | B | 53 | −53.727 | 53.785 | 64.303 | 1.00 | 54.66 | B |
| ATOM | 3770 | C | ARG | B | 53 | −48.778 | 49.893 | 60.266 | 1.00 | 52.44 | B |
| ATOM | 3771 | O | ARG | B | 53 | −48.271 | 49.374 | 61.266 | 1.00 | 53.64 | B |
| ATOM | 3772 | N | SER | B | 54 | −48.079 | 50.635 | 59.410 | 1.00 | 51.86 | B |
| ATOM | 3773 | CA | SER | B | 54 | −46.651 | 50.875 | 59.601 | 1.00 | 53.10 | B |
| ATOM | 3774 | CB | SER | B | 54 | −45.936 | 50.874 | 58.250 | 1.00 | 52.95 | B |
| ATOM | 3775 | OG | SER | B | 54 | −46.095 | 49.633 | 57.582 | 1.00 | 56.07 | B |
| ATOM | 3776 | C | SER | B | 54 | −46.353 | 52.194 | 60.327 | 1.00 | 54.30 | B |
| ATOM | 3777 | O | SER | B | 54 | −47.012 | 53.215 | 60.106 | 1.00 | 55.22 | B |
| ATOM | 3778 | N | VAL | B | 55 | −45.353 | 52.175 | 61.198 | 1.00 | 54.12 | B |
| ATOM | 3779 | CA | VAL | B | 55 | −44.974 | 53.376 | 61.922 | 1.00 | 53.89 | B |
| ATOM | 3780 | CB | VAL | B | 55 | −45.309 | 53.245 | 63.414 | 1.00 | 53.32 | B |
| ATOM | 3781 | CG1 | VAL | B | 55 | −44.532 | 54.252 | 64.227 | 1.00 | 52.81 | B |
| ATOM | 3782 | CG2 | VAL | B | 55 | −46.781 | 53.476 | 63.605 | 1.00 | 53.93 | B |
| ATOM | 3783 | C | VAL | B | 55 | −43.492 | 53.627 | 61.725 | 1.00 | 54.99 | B |
| ATOM | 3784 | O | VAL | B | 55 | −42.658 | 52.770 | 62.005 | 1.00 | 55.61 | B |
| ATOM | 3785 | N | SER | B | 56 | −43.163 | 54.809 | 61.228 | 1.00 | 57.01 | B |
| ATOM | 3786 | CA | SER | B | 56 | −41.772 | 55.140 | 60.972 | 1.00 | 59.04 | B |
| ATOM | 3787 | CB | SER | B | 56 | −41.592 | 55.471 | 59.484 | 1.00 | 60.91 | B |
| ATOM | 3788 | OG | SER | B | 56 | −40.230 | 55.413 | 59.086 | 1.00 | 64.35 | B |
| ATOM | 3789 | C | SER | B | 56 | −41.269 | 56.296 | 61.826 | 1.00 | 58.58 | B |
| ATOM | 3790 | O | SER | B | 56 | −41.239 | 57.431 | 61.367 | 1.00 | 58.25 | B |
| ATOM | 3791 | N | GLN | B | 57 | −40.875 | 56.001 | 63.062 | 1.00 | 59.16 | B |
| ATOM | 3792 | CA | GLN | B | 57 | −40.345 | 57.019 | 63.968 | 1.00 | 61.11 | B |
| ATOM | 3793 | CB | GLN | B | 57 | −41.454 | 57.581 | 64.848 | 1.00 | 60.33 | B |
| ATOM | 3794 | CG | GLN | B | 57 | −42.175 | 56.544 | 65.678 | 1.00 | 62.01 | B |
| ATOM | 3795 | CD | GLN | B | 57 | −43.212 | 57.163 | 66.589 | 1.00 | 62.68 | B |
| ATOM | 3796 | OE1 | GLN | B | 57 | −44.093 | 57.904 | 66.142 | 1.00 | 61.75 | B |
| ATOM | 3797 | NE2 | GLN | B | 57 | −43.113 | 56.862 | 67.879 | 1.00 | 64.87 | B |
| ATOM | 3798 | C | GLN | B | 57 | −39.218 | 56.475 | 64.853 | 1.00 | 62.77 | B |
| ATOM | 3799 | O | GLN | B | 57 | −39.110 | 55.266 | 65.051 | 1.00 | 64.17 | B |
| ATOM | 3800 | N | ASP | B | 58 | −38.378 | 57.377 | 65.364 | 1.00 | 63.99 | B |
| ATOM | 3801 | CA | ASP | B | 58 | −37.251 | 57.035 | 66.247 | 1.00 | 64.75 | B |
| ATOM | 3802 | CB | ASP | B | 58 | −37.773 | 56.430 | 67.556 | 1.00 | 66.84 | B |
| ATOM | 3803 | CG | ASP | B | 58 | −39.066 | 57.099 | 68.047 | 1.00 | 69.34 | B |
| ATOM | 3804 | OD1 | ASP | B | 58 | −40.126 | 56.869 | 67.421 | 1.00 | 70.76 | B |
| ATOM | 3805 | OD2 | ASP | B | 58 | −39.027 | 57.854 | 69.049 | 1.00 | 68.35 | B |
| ATOM | 3806 | C | ASP | B | 58 | −36.212 | 56.095 | 65.643 | .00 | 64.57 | B |
| ATOM | 3807 | O | ASP | B | 58 | −35.837 | 55.099 | 66.261 | .00 | 64.37 | B |
| ATOM | 3808 | N | GLY | B | 59 | −35.735 | 56.420 | 64.446 | 1.00 | 63.88 | B |
| ATOM | 3809 | CA | GLY | B | 59 | −34.746 | 55.583 | 63.788 | .00 | 64.55 | B |
| ATOM | 3810 | C | GLY | B | 59 | −35.247 | 54.164 | 63.607 | 1.00 | 65.16 | B |
| ATOM | 3811 | O | GLY | B | 59 | −34.541 | 53.298 | 63.070 | 1.00 | 66.25 | B |
| ATOM | 3812 | N | LEU | B | 60 | −36.478 | 53.926 | 64.049 | 1.00 | 62.89 | B |
| ATOM | 3813 | CA | LEU | B | 60 | −37.088 | 52.614 | 63.950 | 1.00 | 59.96 | B |
| ATOM | 3814 | CB | LEU | B | 60 | −37.694 | 52.226 | 65.296 | 1.00 | 58.77 | B |
| ATOM | 3815 | CG | LEU | B | 60 | −36.838 | 52.435 | 66.546 | 1.00 | 57.86 | B |
| ATOM | 3816 | CD1 | LEU | B | 60 | −37.655 | 52.121 | 67.792 | 1.00 | 54.26 | B |
| ATOM | 3817 | CD2 | LEU | B | 60 | −35.604 | 51.564 | 66.467 | 1.00 | 57.14 | B |
| ATOM | 3818 | C | LEU | B | 60 | −38.187 | 52.627 | 62.894 | 1.00 | 59.47 | B |
| ATOM | 3819 | O | LEU | B | 60 | −38.712 | 53.678 | 62.536 | 1.00 | 58.86 | B |
| ATOM | 3820 | N | ILE | B | 61 | −38.513 | 51.446 | 62.389 | 1.00 | 58.71 | B |
| ATOM | 3821 | CA | ILE | B | 61 | −39.575 | 51.280 | 61.408 | 1.00 | 59.32 | B |
| ATOM | 3822 | CB | ILE | B | 61 | −39.045 | 51.124 | 59.983 | 1.00 | 58.41 | B |
| ATOM | 3823 | CG2 | ILE | B | 61 | −40.178 | 50.711 | 59.058 | 1.00 | 57.50 | B |
| ATOM | 3824 | CG1 | ILE | B | 61 | −38.410 | 52.425 | 59.518 | 1.00 | 58.13 | B |
| ATOM | 3825 | CD1 | ILE | B | 61 | −37.959 | 52.391 | 58.077 | 1.00 | 58.70 | B |
| ATOM | 3826 | C | ILE | B | 61 | −40.244 | 49.973 | 61.782 | 1.00 | 60.48 | B |
| ATOM | 3827 | O | ILE | B | 61 | −39.619 | 48.911 | 61.692 | 1.00 | 62.67 | B |
| ATOM | 3828 | N | TRP | B | 62 | −41.499 | 50.032 | 62.212 | 1.00 | 59.31 | B |
| ATOM | 3829 | CA | TRP | B | 62 | −42.168 | 48.804 | 62.591 | 1.00 | 59.11 | B |
| ATOM | 3830 | CB | TRP | B | 62 | −42.071 | 48.598 | 64.111 | 1.00 | 59.98 | B |
| ATOM | 3831 | CG | TRP | B | 62 | −43.022 | 49.405 | 64.939 | 1.00 | 61.71 | B |
| ATOM | 3832 | CD2 | TRP | B | 62 | −42.745 | 50.648 | 65.594 | 1.00 | 63.44 | B |
| ATOM | 3833 | CE2 | TRP | B | 62 | −43.917 | 51.025 | 66.287 | 1.00 | 63.69 | B |
| ATOM | 3834 | CE3 | TRP | B | 62 | −41.617 | 51.482 | 65.663 | 1.00 | 63.55 | B |
| ATOM | 3835 | CD1 | TRP | B | 62 | −44.312 | 49.088 | 65.249 | 1.00 | 61.57 | B |
| ATOM | 3836 | NE1 | TRP | B | 62 | −44.857 | 50.054 | 66.060 | 1.00 | 63.09 | B |
| ATOM | 3837 | CZ2 | TRP | B | 62 | −43.994 | 52.201 | 67.043 | 1.00 | 64.68 | B |
| ATOM | 3838 | CZ3 | TRP | B | 62 | −41.694 | 52.651 | 66.414 | 1.00 | 64.59 | B |
| ATOM | 3839 | CH2 | TRP | B | 62 | −42.875 | 52.999 | 67.095 | 1.00 | 65.04 | B |
| ATOM | 3840 | C | TRP | B | 62 | −43.610 | 48.730 | 62.126 | 1.00 | 57.83 | B |
| ATOM | 3841 | O | TRP | B | 62 | −44.168 | 49.717 | 61.650 | 1.00 | 56.68 | B |
| ATOM | 3842 | N | ASP | B | 63 | −44.179 | 47.530 | 62.233 | 1.00 | 56.20 | B |
| ATOM | 3843 | CA | ASP | B | 63 | −45.555 | 47.265 | 61.855 | 1.00 | 55.38 | B |
| ATOM | 3844 | CB | ASP | B | 63 | −45.612 | 46.148 | 60.806 | 1.00 | 54.77 | B |
| ATOM | 3845 | CG | ASP | B | 63 | −45.461 | 46.671 | 59.374 | 1.00 | 55.77 | B |
| ATOM | 3846 | OD1 | ASP | B | 63 | −46.218 | 47.581 | 58.995 | 1.00 | 54.29 | B |
| ATOM | 3847 | OD2 | ASP | B | 63 | −44.602 | 46.174 | 58.610 | 1.00 | 56.94 | B |
| ATOM | 3848 | C | ASP | B | 63 | −46.341 | 46.859 | 63.104 | 1.00 | 55.75 | B |
| ATOM | 3849 | O | ASP | B | 63 | −45.983 | 45.907 | 63.804 | 1.00 | 56.09 | B |
| ATOM | 3850 | N | GLU | B | 64 | −47.408 | 47.596 | 63.392 | 1.00 | 55.40 | B |
| ATOM | 3851 | CA | GLU | B | 64 | −48.253 | 47.312 | 64.551 | 1.00 | 54.56 | B |
| ATOM | 3852 | CB | GLU | B | 64 | −48.922 | 48.595 | 65.025 | 1.00 | 53.75 | B |
| ATOM | 3853 | CG | GLU | B | 64 | −47.938 | 49.726 | 65.169 | 1.00 | 56.32 | B |
| ATOM | 3854 | CD | GLU | B | 64 | −48.555 | 50.999 | 65.684 | 1.00 | 57.01 | B |
| ATOM | 3855 | OE1 | GLU | B | 64 | −49.364 | 51.632 | 64.961 | 1.00 | 57.05 | B |
| ATOM | 3856 | OE2 | GLU | B | 64 | −48.217 | 51.364 | 66.825 | 1.00 | 58.36 | B |
| ATOM | 3857 | C | GLU | B | 64 | −49.314 | 46.286 | 64.167 | 1.00 | 55.14 | B |
| ATOM | 3858 | O | GLU | B | 64 | −50.021 | 45.761 | 65.028 | 1.00 | 55.05 | B |
| ATOM | 3859 | N | GLY | B | 65 | −49.419 | 46.018 | 62.862 | 1.00 | 55.06 | B |
| ATOM | 3860 | CA | GLY | B | 65 | −50.373 | 45.051 | 62.361 | 1.00 | 54.52 | B |
| ATOM | 3861 | C | GLY | B | 65 | −49.684 | 43.911 | 61.631 | 1.00 | 55.16 | B |
| ATOM | 3862 | O | GLY | B | 65 | −48.762 | 43.294 | 62.161 | 1.00 | 55.80 | B |
| ATOM | 3863 | N | ALA | B | 66 | −50.123 | 43.620 | 60.410 | 1.00 | 55.64 | B |
| ATOM | 3864 | CA | ALA | B | 66 | −49.523 | 42.540 | 59.639 | 1.00 | 55.41 | B |
| ATOM | 3865 | CB | ALA | B | 66 | −50.260 | 42.367 | 58.332 | 1.00 | 56.61 | B |
| ATOM | 3866 | C | ALA | B | 66 | −48.060 | 42.851 | 59.387 | 1.00 | 55.56 | B |
| ATOM | 3867 | O | ALA | B | 66 | −47.731 | 43.937 | 58.930 | 1.00 | 55.46 | B |
| ATOM | 3868 | N | ASN | B | 67 | −47.188 | 41.895 | 59.679 | 1.00 | 56.49 | B |
| ATOM | 3869 | CA | ASN | B | 67 | −45.750 | 42.100 | 59.510 | 1.00 | 59.65 | B |
| ATOM | 3870 | CB | ASN | B | 67 | −44.996 | 41.465 | 60.676 | 1.00 | 60.40 | B |
| ATOM | 3871 | CG | ASN | B | 67 | −44.580 | 42.475 | 61.710 | 1.00 | 62.03 | B |
| ATOM | 3872 | OD1 | ASN | B | 67 | −43.405 | 42.846 | 61.789 | 1.00 | 62.97 | B |
| ATOM | 3873 | ND2 | ASN | B | 67 | −45.543 | 42.941 | 62.508 | 1.00 | 61.37 | B |
| ATOM | 3874 | C | ASN | B | 67 | −45.172 | 41.566 | 58.218 | 1.00 | 59.97 | B |
| ATOM | 3875 | O | ASN | B | 67 | −44.203 | 42.098 | 57.681 | 1.00 | 60.20 | B |
| ATOM | 3876 | N | THR | B | 68 | −45.768 | 40.502 | 57.722 | 1.00 | 61.28 | B |
| ATOM | 3877 | CA | THR | B | 68 | −45.280 | 39.911 | 56.509 | 1.00 | 62.87 | B |
| ATOM | 3878 | CB | THR | B | 68 | −44.312 | 38.781 | 56.847 | 1.00 | 62.58 | B |
| ATOM | 3879 | OG1 | THR | B | 68 | −43.647 | 38.372 | 55.652 | 1.00 | 66.70 | B |
| ATOM | 3880 | CG2 | THR | B | 68 | −45.048 | 37.600 | 57.468 | 1.00 | 61.61 | B |
| ATOM | 3881 | C | THR | B | 68 | −46.452 | 39.411 | 55.680 | 1.00 | 64.13 | B |
| ATOM | 3882 | O | THR | B | 68 | −47.586 | 39.430 | 56.135 | 1.00 | 63.52 | B |
| ATOM | 3883 | N | MSE | B | 69 | −46.181 | 38.966 | 54.462 | 1.00 | 66.75 | B |
| ATOM | 3884 | CA | MSE | B | 69 | −47.239 | 38.506 | 53.577 | 1.00 | 69.72 | B |
| ATOM | 3885 | CB | MSE | B | 69 | −47.904 | 39.733 | 52.955 | 1.00 | 72.00 | B |
| ATOM | 3886 | CG | MSE | B | 69 | −48.671 | 39.495 | 51.676 | 1.00 | 76.92 | B |
| ATOM | 3887 | SE | MSE | B | 69 | −48.821 | 41.135 | 50.695 | 1.00 | 82.86 | B |
| ATOM | 3888 | CE | MSE | B | 69 | −47.215 | 41.942 | 51.352 | 1.00 | 79.42 | B |
| ATOM | 3889 | C | MSE | B | 69 | −46.656 | 37.603 | 52.502 | 1.00 | 70.57 | B |
| ATOM | 3890 | O | MSE | B | 69 | −45.441 | 37.440 | 52.419 | 1.00 | 71.22 | B |
| ATOM | 3891 | N | THR | B | 70 | −47.517 | 37.004 | 51.686 | 1.00 | 72.29 | B |
| ATOM | 3892 | CA | THR | B | 70 | −47.058 | 36.128 | 50.605 | 1.00 | 74.05 | B |
| ATOM | 3893 | CB | THR | B | 70 | −47.067 | 34.663 | 51.054 | 1.00 | 72.73 | B |
| ATOM | 3894 | OG1 | THR | B | 70 | −46.106 | 34.487 | 52.103 | 1.00 | 71.53 | B |
| ATOM | 3895 | CG2 | THR | B | 70 | −46.717 | 33.750 | 49.898 | 1.00 | 73.43 | B |
| ATOM | 3896 | C | THR | B | 70 | −47.932 | 36.266 | 49.361 | 1.00 | 75.32 | B |
| ATOM | 3897 | O | THR | B | 70 | −49.050 | 36.769 | 49.436 | 1.00 | 77.63 | B |
| ATOM | 3898 | N | GLU | B | 71 | −47.432 | 35.844 | 48.207 | 1.00 | 75.88 | B |
| ATOM | 3899 | CA | GLU | B | 71 | −48.251 | 35.940 | 47.013 | 1.00 | 76.72 | B |
| ATOM | 3900 | CB | GLU | B | 71 | −47.573 | 36.808 | 45.945 | 1.00 | 76.31 | B |
| ATOM | 3901 | CG | GLU | B | 71 | −46.266 | 36.300 | 45.389 | 1.00 | 77.07 | B |
| ATOM | 3902 | CD | GLU | B | 71 | −45.600 | 37.341 | 44.507 | 1.00 | 78.85 | B |
| ATOM | 3903 | OE1 | GLU | B | 71 | −44.778 | 38.121 | 45.038 | 1.00 | 79.17 | B |
| ATOM | 3904 | OE2 | GLU | B | 71 | −45.914 | 37.399 | 43.291 | 1.00 | 79.32 | B |
| ATOM | 3905 | C | GLU | B | 71 | −48.563 | 34.552 | 46.490 | 1.00 | 77.55 | B |
| ATOM | 3906 | O | GLU | B | 71 | −48.101 | 34.140 | 45.428 | 1.00 | 78.55 | B |
| ATOM | 3907 | N | SER | B | 72 | −49.343 | 33.825 | 47.281 | 1.00 | 78.72 | B |
| ATOM | 3908 | CA | SER | B | 72 | −49.769 | 32.473 | 46.938 | 1.00 | 79.47 | B |
| ATOM | 3909 | CB | SER | B | 72 | −50.003 | 31.647 | 48.206 | 1.00 | 80.70 | B |
| ATOM | 3910 | OG | SER | B | 72 | −48.930 | 31.782 | 49.128 | 1.00 | 82.79 | B |
| ATOM | 3911 | C | SER | B | 72 | −51.089 | 32.644 | 46.203 | 1.00 | 79.54 | B |
| ATOM | 3912 | O | SER | B | 72 | −51.693 | 31.680 | 45.738 | 1.00 | 79.45 | B |
| ATOM | 3913 | N | GLU | B | 73 | −51.530 | 33.896 | 46.121 | 1.00 | 79.63 | B |
| ATOM | 3914 | CA | GLU | B | 73 | −52.768 | 34.247 | 45.445 | 1.00 | 79.13 | B |
| ATOM | 3915 | CB | GLU | B | 73 | −53.612 | 35.141 | 46.346 | 1.00 | 79.71 | B |
| ATOM | 3916 | CG | GLU | B | 73 | −53.806 | 34.582 | 47.730 | 1.00 | 81.19 | B |
| ATOM | 3917 | CD | GLU | B | 73 | −54.461 | 33.222 | 47.700 | 1.00 | 83.67 | B |
| ATOM | 3918 | OE1 | GLU | B | 73 | −55.606 | 33.128 | 47.201 | 1.00 | 84.54 | B |
| ATOM | 3919 | OE2 | GLU | B | 73 | −53.830 | 32.249 | 48.171 | 1.00 | 84.48 | B |
| ATOM | 3920 | C | GLU | B | 73 | −52.427 | 34.987 | 44.154 | 1.00 | 78.59 | B |
| ATOM | 3921 | O | GLU | B | 73 | −51.527 | 35.834 | 44.128 | 1.00 | 77.63 | B |
| ATOM | 3922 | N | GLY | B | 74 | −53.146 | 34.659 | 43.086 | 1.00 | 78.11 | B |
| ATOM | 3923 | CA | GLY | B | 74 | −52.898 | 35.305 | 41.813 | 1.00 | 77.86 | B |
| ATOM | 3924 | C | GLY | B | 74 | −53.092 | 36.801 | 41.917 | 1.00 | 78.34 | B |
| ATOM | 3925 | O | GLY | B | 74 | −52.291 | 37.591 | 41.395 | 1.00 | 78.30 | B |
| ATOM | 3926 | N | ASP | B | 75 | −54.157 | 37.195 | 42.605 | 1.00 | 77.54 | B |
| ATOM | 3927 | CA | ASP | B | 75 | −54.456 | 38.605 | 42.775 | 1.00 | 76.95 | B |
| ATOM | 3928 | CB | ASP | B | 75 | −55.759 | 38.769 | 43.546 | 1.00 | 79.29 | B |
| ATOM | 3929 | CG | ASP | B | 75 | −56.960 | 38.302 | 42.755 | 1.00 | 81.65 | B |
| ATOM | 3930 | OD1 | ASP | B | 75 | −57.153 | 38.794 | 41.616 | 1.00 | 82.99 | B |
| ATOM | 3931 | OD2 | ASP | B | 75 | −57.710 | 37.448 | 43.276 | 1.00 | 82.48 | B |
| ATOM | 3932 | C | ASP | B | 75 | −53.348 | 39.392 | 43.467 | 1.00 | 75.86 | B |
| ATOM | 3933 | O | ASP | B | 75 | −53.177 | 40.583 | 43.203 | 1.00 | 76.01 | B |
| ATOM | 3934 | N | VAL | B | 76 | −52.599 | 38.745 | 44.357 | 1.00 | 73.99 | B |
| ATOM | 3935 | CA | VAL | B | 76 | −51.526 | 39.447 | 45.050 | 1.00 | 71.79 | B |
| ATOM | 3936 | CB | VAL | B | 76 | −50.880 | 38.609 | 46.164 | 1.00 | 70.27 | B |
| ATOM | 3937 | CG1 | VAL | B | 76 | −49.677 | 39.346 | 46.713 | 1.00 | 68.04 | B |
| ATOM | 3938 | CG2 | VAL | B | 76 | −51.877 | 38.347 | 47.272 | 1.00 | 68.91 | B |
| ATOM | 3939 | C | VAL | B | 76 | −50.443 | 39.797 | 44.062 | 1.00 | 72.23 | B |
| ATOM | 3940 | O | VAL | B | 76 | −49.908 | 40.899 | 44.095 | 1.00 | 72.64 | B |
| ATOM | 3941 | N | THR | B | 77 | −50.116 | 38.854 | 43.183 | 1.00 | 72.74 | B |
| ATOM | 3942 | CA | THR | B | 77 | −49.074 | 39.097 | 42.193 | 1.00 | 73.35 | B |
| ATOM | 3943 | CB | THR | B | 77 | −48.730 | 37.823 | 41.403 | 1.00 | 73.33 | B |
| ATOM | 3944 | OG1 | THR | B | 77 | −48.249 | 36.820 | 42.307 | 1.00 | 73.09 | B |
| ATOM | 3945 | CG2 | THR | B | 77 | −47.658 | 38.123 | 40.360 | 1.00 | 71.74 | B |
| ATOM | 3946 | C | THR | B | 77 | −49.534 | 40.176 | 41.230 | 1.00 | 73.21 | B |
| ATOM | 3947 | O | THR | B | 77 | −48.757 | 41.062 | 40.859 | 1.00 | 73.42 | B |
| ATOM | 3948 | N | PHE | B | 78 | −50.803 | 40.098 | 40.838 | 1.00 | 72.46 | B |
| ATOM | 3949 | CA | PHE | B | 78 | −51.380 | 41.077 | 39.931 | 1.00 | 72.19 | B |
| ATOM | 3950 | CB | PHE | B | 78 | −52.856 | 40.761 | 39.661 | 1.00 | 72.54 | B |
| ATOM | 3951 | CG | PHE | B | 78 | −53.609 | 41.901 | 39.031 | 1.00 | 72.50 | B |
| ATOM | 3952 | CD1 | PHE | B | 78 | −54.094 | 42.947 | 39.811 | 1.00 | 71.76 | B |
| ATOM | 3953 | CD2 | PHE | B | 78 | −53.783 | 41.956 | 37.653 | 1.00 | 71.90 | B |
| ATOM | 3954 | CE1 | PHE | B | 78 | −54.738 | 44.033 | 39.225 | 1.00 | 73.09 | B |
| ATOM | 3955 | CE2 | PHE | B | 78 | −54.427 | 43.042 | 37.060 | 1.00 | 73.06 | B |
| ATOM | 3956 | CZ | PHE | B | 78 | −54.905 | 44.083 | 37.847 | 1.00 | 72.11 | B |
| ATOM | 3957 | C | PHE | B | 78 | −51.258 | 42.485 | 40.501 | 1.00 | 72.14 | B |
| ATOM | 3958 | O | PHE | B | 78 | −50.806 | 43.399 | 39.808 | 1.00 | 72.00 | B |
| ATOM | 3959 | N | LEU | B | 79 | −51.667 | 42.662 | 41.758 | 1.00 | 71.78 | B |
| ATOM | 3960 | CA | LEU | B | 79 | −51.590 | 43.975 | 42.392 | 1.00 | 71.86 | B |
| ATOM | 3961 | CB | LEU | B | 79 | −52.303 | 43.980 | 43.744 | 1.00 | 71.50 | B |
| ATOM | 3962 | CG | LEU | B | 79 | −53.826 | 43.921 | 43.645 | 1.00 | 73.12 | B |
| ATOM | 3963 | CD1 | LEU | B | 79 | −54.446 | 44.087 | 45.026 | 1.00 | 73.47 | B |
| ATOM | 3964 | CD2 | LEU | B | 79 | −54.310 | 45.018 | 42.713 | 1.00 | 72.72 | B |
| ATOM | 3965 | C | LEU | B | 79 | −50.164 | 44.465 | 42.572 | 1.00 | 71.41 | B |
| ATOM | 3966 | O | LEU | B | 79 | −49.885 | 45.643 | 42.360 | 1.00 | 71.39 | B |
| ATOM | 3967 | N | ILE | B | 80 | −49.258 | 43.574 | 42.960 | 1.00 | 71.38 | B |
| ATOM | 3968 | CA | ILE | B | 80 | −47.874 | 43.981 | 43.141 | 1.00 | 72.39 | B |
| ATOM | 3969 | CB | ILE | B | 80 | −46.961 | 42.804 | 43.503 | 1.00 | 72.60 | B |
| ATOM | 3970 | CG2 | ILE | B | 80 | −45.522 | 43.298 | 43.656 | 1.00 | 71.20 | B |
| ATOM | 3971 | CG1 | ILE | B | 80 | −47.431 | 42.158 | 44.806 | 1.00 | 73.44 | B |
| ATOM | 3972 | CD1 | ILE | B | 80 | −47.373 | 43.083 | 46.010 | 1.00 | 74.84 | B |
| ATOM | 3973 | C | ILE | B | 80 | −47.359 | 44.584 | 41.846 | 1.00 | 73.69 | B |
| ATOM | 3974 | O | ILE | B | 80 | −46.552 | 45.512 | 41.875 | 1.00 | 74.40 | B |
| ATOM | 3975 | N | ASP | B | 81 | −47.832 | 44.054 | 40.715 | 1.00 | 74.56 | B |
| ATOM | 3976 | CA | ASP | B | 81 | −47.426 | 44.532 | 39.389 | 1.00 | 74.59 | B |
| ATOM | 3977 | CB | ASP | B | 81 | −47.634 | 43.428 | 38.354 | 1.00 | 75.54 | B |
| ATOM | 3978 | CG | ASP | B | 81 | −46.849 | 42.167 | 38.685 | 1.00 | 78.18 | B |
| ATOM | 3979 | OD1 | ASP | B | 81 | −45.718 | 42.294 | 39.210 | 1.00 | 78.20 | B |
| ATOM | 3980 | OD2 | ASP | B | 81 | −47.353 | 41.051 | 38.413 | 1.00 | 79.22 | B |
| ATOM | 3981 | C | ASP | B | 81 | −48.138 | 45.815 | 38.933 | 1.00 | 74.07 | B |
| ATOM | 3982 | O | ASP | B | 81 | −47.488 | 46.764 | 38.491 | 1.00 | 72.18 | B |
| ATOM | 3983 | N | SER | B | 82 | −49.465 | 45.845 | 39.038 | 1.00 | 74.34 | B |
| ATOM | 3984 | CA | SER | B | 82 | −50.219 | 47.032 | 38.640 | 1.00 | 75.09 | B |
| ATOM | 3985 | CB | SER | B | 82 | −51.695 | 46.909 | 39.026 | 1.00 | 75.42 | B |
| ATOM | 3986 | OG | SER | B | 82 | −52.404 | 46.073 | 38.125 | 1.00 | 77.99 | B |
| ATOM | 3987 | C | SER | B | 82 | −49.632 | 48.245 | 39.327 | 1.00 | 75.44 | B |
| ATOM | 3988 | O | SER | B | 82 | −49.121 | 49.151 | 38.679 | 1.00 | 76.74 | B |
| ATOM | 3989 | N | LEU | B | 83 | −49.698 | 48.252 | 40.651 | 1.00 | 75.68 | B |
| ATOM | 3990 | CA | LEU | B | 83 | −49.176 | 49.358 | 41.437 | 1.00 | 75.09 | B |
| ATOM | 3991 | CB | LEU | B | 83 | −49.552 | 49.160 | 42.905 | 1.00 | 75.15 | B |
| ATOM | 3992 | CG | LEU | B | 83 | −51.040 | 48.924 | 43.181 | 1.00 | 75.26 | B |
| ATOM | 3993 | CD1 | LEU | B | 83 | −51.227 | 48.513 | 44.644 | 1.00 | 76.49 | B |
| ATOM | 3994 | CD2 | LEU | B | 83 | −51.834 | 50.177 | 42.857 | 1.00 | 74.32 | B |
| ATOM | 3995 | C | LEU | B | 83 | −47.655 | 49.505 | 41.301 | 1.00 | 75.00 | B |
| ATOM | 3996 | O | LEU | B | 83 | −47.077 | 50.469 | 41.803 | 1.00 | 75.92 | B |
| ATOM | 3997 | N | GLY | B | 84 | −47.012 | 48.558 | 40.621 | 1.00 | 74.12 | B |
| ATOM | 3998 | CA | GLY | B | 84 | −45.567 | 48.620 | 40.446 | 1.00 | 74.17 | B |
| ATOM | 3999 | C | GLY | B | 84 | −44.788 | 48.696 | 41.754 | 1.00 | 74.42 | B |
| ATOM | 4000 | O | GLY | B | 84 | −44.001 | 49.620 | 41.973 | 1.00 | 74.25 | B |
| ATOM | 4001 | N | LEU | B | 85 | −44.991 | 47.709 | 42.620 | 1.00 | 74.09 | B |
| ATOM | 4002 | CA | LEU | B | 85 | −44.333 | 47.674 | 43.916 | 1.00 | 73.95 | B |
| ATOM | 4003 | CB | LEU | B | 85 | −45.363 | 47.324 | 45.005 | 1.00 | 73.73 | B |
| ATOM | 4004 | CG | LEU | B | 85 | −46.446 | 48.388 | 45.255 | 1.00 | 73.40 | B |
| ATOM | 4005 | CD1 | LEU | B | 85 | −47.560 | 47.858 | 46.150 | 1.00 | 71.76 | B |
| ATOM | 4006 | CD2 | LEU | B | 85 | −45.784 | 49.605 | 45.888 | 1.00 | 74.62 | B |
| ATOM | 4007 | C | LEU | B | 85 | −43.160 | 46.704 | 43.984 | 1.00 | 74.13 | B |
| ATOM | 4008 | O | LEU | B | 85 | −42.580 | 46.509 | 45.054 | 1.00 | 74.04 | B |
| ATOM | 4009 | N | ARG | B | 86 | −42.799 | 46.107 | 42.852 | 1.00 | 74.26 | B |
| ATOM | 4010 | CA | ARG | B | 86 | −41.693 | 45.148 | 42.834 | 1.00 | 74.74 | B |
| ATOM | 4011 | CB | ARG | B | 86 | −41.370 | 44.695 | 41.411 | 1.00 | 75.37 | B |
| ATOM | 4012 | CG | ARG | B | 86 | −42.376 | 43.734 | 40.823 | 1.00 | 77.70 | B |
| ATOM | 4013 | CD | ARG | B | 86 | −42.467 | 42.456 | 41.648 | 1.00 | 78.93 | B |
| ATOM | 4014 | NE | ARG | B | 86 | −43.512 | 41.566 | 41.146 | 1.00 | 80.42 | B |
| ATOM | 4015 | CZ | ARG | B | 86 | −43.932 | 40.475 | 41.777 | 1.00 | 81.40 | B |
| ATOM | 4016 | NH1 | ARG | B | 86 | −43.392 | 40.130 | 42.941 | 1.00 | 80.81 | B |
| ATOM | 4017 | NH2 | ARG | B | 86 | −44.907 | 39.738 | 41.251 | 1.00 | 82.27 | B |
| ATOM | 4018 | C | ARG | B | 86 | −40.427 | 45.685 | 43.461 | 1.00 | 74.19 | B |
| ATOM | 4019 | O | ARG | B | 86 | −39.777 | 44.984 | 44.232 | 1.00 | 74.33 | B |
| ATOM | 4020 | N | GLU | B | 87 | −40.078 | 46.925 | 43.127 | 1.00 | 74.08 | B |
| ATOM | 4021 | CA | GLU | B | 87 | −38.871 | 47.550 | 43.663 | 1.00 | 73.63 | B |
| ATOM | 4022 | CB | GLU | B | 87 | −38.618 | 48.893 | 42.974 | 1.00 | 73.85 | B |
| ATOM | 4023 | CG | GLU | B | 87 | −38.337 | 48.789 | 41.484 | 1.00 | 74.07 | B |
| ATOM | 4024 | CD | GLU | B | 87 | −38.073 | 50.138 | 40.844 | 1.00 | 74.17 | B |
| ATOM | 4025 | OE1 | GLU | B | 87 | −37.823 | 50.180 | 39.621 | 1.00 | 74.22 | B |
| ATOM | 4026 | OE2 | GLU | B | 87 | −38.114 | 51.158 | 41.564 | 1.00 | 74.22 | B |
| ATOM | 4027 | C | GLU | B | 87 | −38.951 | 47.762 | 45.173 | 1.00 | 73.33 | B |
| ATOM | 4028 | O | GLU | B | 87 | −37.925 | 47.794 | 45.856 | 1.00 | 71.37 | B |
| ATOM | 4029 | N | LYS | B | 88 | −40.175 | 47.898 | 45.688 | 1.00 | 74.03 | B |
| ATOM | 4030 | CA | LYS | B | 88 | −40.402 | 48.118 | 47.118 | 1.00 | 72.89 | B |
| ATOM | 4031 | CB | LYS | B | 88 | −41.579 | 49.082 | 47.322 | 1.00 | 72.50 | B |
| ATOM | 4032 | CG | LYS | B | 88 | −41.269 | 50.512 | 46.873 | 1.00 | 72.99 | B |
| ATOM | 4033 | CD | LYS | B | 88 | −42.492 | 51.406 | 46.941 | 1.00 | 73.63 | B |
| ATOM | 4034 | CE | LYS | B | 88 | −42.164 | 52.842 | 46.557 | 1.00 | 72.62 | B |
| ATOM | 4035 | NZ | LYS | B | 88 | −43.298 | 53.775 | 46.852 | 1.00 | 71.06 | B |
| ATOM | 4036 | C | LYS | B | 88 | −40.629 | 46.826 | 47.892 | 1.00 | 71.79 | B |
| ATOM | 4037 | O | LYS | B | 88 | −40.700 | 46.837 | 49.117 | 1.00 | 71.18 | B |
| ATOM | 4038 | N | GLN | B | 89 | −40.733 | 45.715 | 47.172 | 1.00 | 71.50 | B |
| ATOM | 4039 | CA | GLN | B | 89 | −40.919 | 44.417 | 47.805 | 1.00 | 71.71 | B |
| ATOM | 4040 | CB | GLN | B | 89 | −41.217 | 43.352 | 46.756 | 1.00 | 72.88 | B |
| ATOM | 4041 | CG | GLN | B | 89 | −41.825 | 42.080 | 47.334 | 1.00 | 75.03 | B |
| ATOM | 4042 | CD | GLN | B | 89 | −42.114 | 41.034 | 46.272 | 1.00 | 75.52 | B |
| ATOM | 4043 | OE1 | GLN | B | 89 | −42.849 | 40.075 | 46.511 | 1.00 | 75.84 | B |
| ATOM | 4044 | NE2 | GLN | B | 89 | −41.530 | 41.213 | 45.091 | 1.00 | 76.33 | B |
| ATOM | 4045 | C | GLN | B | 89 | −39.620 | 44.071 | 48.522 | 1.00 | 71.40 | B |
| ATOM | 4046 | O | GLN | B | 89 | −38.544 | 44.094 | 47.917 | 1.00 | 71.92 | B |
| ATOM | 4047 | N | GLN | B | 90 | −39.724 | 43.737 | 49.803 | 1.00 | 70.30 | B |
| ATOM | 4048 | CA | GLN | B | 90 | −38.552 | 43.427 | 50.609 | 1.00 | 69.58 | B |
| ATOM | 4049 | CB | GLN | B | 90 | −38.339 | 44.564 | 51.631 | 1.00 | 70.69 | B |
| ATOM | 4050 | CG | GLN | B | 90 | −37.334 | 44.313 | 52.759 | 1.00 | 72.49 | B |
| ATOM | 4051 | CD | GLN | B | 90 | −37.030 | 45.578 | 53.574 | 1.00 | 74.70 | B |
| ATOM | 4052 | OE1 | GLN | B | 90 | −37.932 | 46.351 | 53.916 | 1.00 | 75.01 | B |
| ATOM | 4053 | NE2 | GLN | B | 90 | −35.753 | 45.785 | 53.894 | 1.00 | 75.54 | B |
| ATOM | 4054 | C | GLN | B | 90 | −38.624 | 42.063 | 51.302 | 1.00 | 68.82 | B |
| ATOM | 4055 | O | GLN | B | 90 | −39.575 | 41.762 | 52.030 | 1.00 | 67.73 | B |
| ATOM | 4056 | N | PHE | B | 91 | −37.606 | 41.240 | 51.052 | 1.00 | 67.78 | B |
| ATOM | 4057 | CA | PHE | B | 91 | −37.505 | 39.915 | 51.646 | 1.00 | 67.03 | B |
| ATOM | 4058 | CB | PHE | B | 91 | −36.977 | 38.908 | 50.629 | 1.00 | 66.80 | B |
| ATOM | 4059 | CG | PHE | B | 91 | −37.843 | 38.771 | 49.424 | 1.00 | 68.28 | B |
| ATOM | 4060 | CD1 | PHE | B | 91 | −37.748 | 39.685 | 48.378 | 1.00 | 70.14 | B |
| ATOM | 4061 | CD2 | PHE | B | 91 | −38.790 | 37.753 | 49.347 | 1.00 | 68.20 | B |
| ATOM | 4062 | CE1 | PHE | B | 91 | −38.591 | 39.587 | 47.264 | 1.00 | 71.20 | B |
| ATOM | 4063 | CE2 | PHE | B | 91 | −39.637 | 37.644 | 48.244 | 1.00 | 68.65 | B |
| ATOM | 4064 | CZ | PHE | B | 91 | −39.539 | 38.561 | 47.200 | 1.00 | 69.84 | B |
| ATOM | 4065 | C | PHE | B | 91 | −36.579 | 39.941 | 52.852 | 1.00 | 66.83 | B |
| ATOM | 4066 | O | PHE | B | 91 | −35.780 | 40.864 | 53.020 | 1.00 | 67.49 | B |
| ATOM | 4067 | N | PRO | B | 92 | −36.680 | 38.928 | 53.721 | 1.00 | 66.21 | B |
| ATOM | 4068 | CD | PRO | B | 92 | −37.718 | 37.887 | 53.801 | 1.00 | 64.67 | B |
| ATOM | 4069 | CA | PRO | B | 92 | −35.817 | 38.897 | 54.905 | 1.00 | 66.55 | B |
| ATOM | 4070 | CB | PRO | B | 92 | −36.410 | 37.757 | 55.734 | 1.00 | 66.15 | B |
| ATOM | 4071 | CG | PRO | B | 92 | −37.849 | 37.707 | 55.280 | 1.00 | 65.28 | B |
| ATOM | 4072 | C | PRO | B | 92 | −34.372 | 38.629 | 54.504 | 1.00 | 67.01 | B |
| ATOM | 4073 | O | PRO | B | 92 | −34.120 | 37.894 | 53.551 | 1.00 | 67.13 | B |
| ATOM | 4074 | N | ALA | B | 93 | −33.426 | 39.230 | 55.217 | 1.00 | 67.68 | B |
| ATOM | 4075 | CA | ALA | B | 93 | −32.020 | 39.021 | 54.906 | 1.00 | 68.60 | B |
| ATOM | 4076 | CB | ALA | B | 93 | −31.202 | 40.251 | 55.257 | 1.00 | 67.34 | B |
| ATOM | 4077 | C | ALA | B | 93 | −31.536 | 37.818 | 55.698 | 1.00 | 70.66 | B |
| ATOM | 4078 | O | ALA | B | 93 | −30.595 | 37.130 | 55.293 | 1.00 | 71.92 | B |
| ATOM | 4079 | N | SER | B | 94 | −32.190 | 37.550 | 56.824 | 1.00 | 71.90 | B |
| ATOM | 4080 | CA | SER | B | 94 | −31.809 | 36.409 | 57.656 | 1.00 | 73.01 | B |
| ATOM | 4081 | CB | SER | B | 94 | −32.300 | 36.607 | 59.093 | 1.00 | 72.53 | B |
| ATOM | 4082 | OG | SER | B | 94 | −33.702 | 36.802 | 59.133 | 1.00 | 73.89 | B |
| ATOM | 4083 | C | SER | B | 94 | −32.347 | 35.087 | 57.106 | 1.00 | 73.82 | B |
| ATOM | 4084 | O | SER | B | 94 | −33.251 | 35.065 | 56.266 | 1.00 | 73.57 | B |
| ATOM | 4085 | N | GLN | B | 95 | −31.776 | 33.988 | 57.589 | 1.00 | 75.46 | B |
| ATOM | 4086 | CA | GLN | B | 95 | −32.174 | 32.648 | 57.168 | 1.00 | 77.02 | B |
| ATOM | 4087 | CB | GLN | B | 95 | −31.208 | 31.614 | 57.722 | 1.00 | 80.58 | B |
| ATOM | 4088 | CG | GLN | B | 95 | −29.760 | 31.801 | 57.347 | 1.00 | 85.63 | B |
| ATOM | 4089 | CD | GLN | B | 95 | −28.873 | 30.835 | 58.112 | 1.00 | 88.88 | B |
| ATOM | 4090 | OE1 | GLN | B | 95 | −28.644 | 31.007 | 59.319 | 1.00 | 90.19 | B |
| ATOM | 4091 | NE2 | GLN | B | 95 | −28.391 | 29.796 | 57.425 | 1.00 | 88.75 | B |
| ATOM | 4092 | C | GLN | B | 95 | −33.560 | 32.290 | 57.688 | 1.00 | 76.24 | B |
| ATOM | 4093 | O | GLN | B | 95 | −33.877 | 32.563 | 58.848 | 1.00 | 75.63 | B |
| ATOM | 4094 | N | ASN | B | 96 | −34.377 | 31.661 | 56.846 | 1.00 | 74.67 | B |
| ATOM | 4095 | CA | ASN | B | 96 | −35.702 | 31.264 | 57.288 | 1.00 | 72.87 | B |
| ATOM | 4096 | CB | ASN | B | 96 | −36.591 | 30.890 | 56.091 | 1.00 | 74.31 | B |
| ATOM | 4097 | CG | ASN | B | 96 | −36.417 | 29.446 | 55.648 | 1.00 | 76.28 | B |
| ATOM | 4098 | OD1 | ASN | B | 96 | −35.298 | 28.974 | 55.433 | 1.00 | 76.91 | B |
| ATOM | 4099 | ND2 | ASN | B | 96 | −37.533 | 28.737 | 55.502 | 1.00 | 76.35 | B |
| ATOM | 4100 | C | ASN | B | 96 | −35.537 | 30.088 | 58.268 | 1.00 | 71.09 | B |
| ATOM | 4101 | O | ASN | B | 96 | −35.405 | 28.923 | 57.887 | 1.00 | 70.21 | B |
| ATOM | 4102 | N | LYS | B | 97 | −35.502 | 30.431 | 59.548 | 1.00 | 68.43 | B |
| ATOM | 4103 | CA | LYS | B | 97 | −35.355 | 29.460 | 60.610 | 1.00 | 65.86 | B |
| ATOM | 4104 | CB | LYS | B | 97 | −33.906 | 29.421 | 61.100 | 1.00 | 66.26 | B |
| ATOM | 4105 | CG | LYS | B | 97 | −32.882 | 29.259 | 59.986 | 1.00 | 68.90 | B |
| ATOM | 4106 | CD | LYS | B | 97 | −31.453 | 29.457 | 60.488 | 1.00 | 71.40 | B |
| ATOM | 4107 | CE | LYS | B | 97 | −31.024 | 28.368 | 61.474 | 1.00 | 72.33 | B |
| ATOM | 4108 | NZ | LYS | B | 97 | −29.701 | 28.673 | 62.110 | 1.00 | 72.92 | B |
| ATOM | 4109 | C | LYS | B | 97 | −36.256 | 29.976 | 61.710 | 1.00 | 64.19 | B |
| ATOM | 4110 | O | LYS | B | 97 | −36.133 | 31.122 | 62.131 | 1.00 | 64.42 | B |
| ATOM | 4111 | N | ARG | B | 98 | −37.186 | 29.147 | 62.154 | 1.00 | 61.78 | B |
| ATOM | 4112 | CA | ARG | B | 98 | −38.089 | 29.543 | 63.215 | 1.00 | 58.56 | B |
| ATOM | 4113 | CB | ARG | B | 98 | −39.538 | 29.289 | 62.806 | 1.00 | 58.90 | B |
| ATOM | 4114 | CG | ARG | B | 98 | −39.960 | 29.995 | 61.542 | 1.00 | 61.82 | B |
| ATOM | 4115 | CD | ARG | B | 98 | −41.370 | 29.582 | 61.144 | 1.00 | 68.45 | B |
| ATOM | 4116 | NE | ARG | B | 98 | −41.918 | 30.443 | 60.099 | 1.00 | 73.22 | B |
| ATOM | 4117 | CZ | ARG | B | 98 | −41.524 | 30.429 | 58.829 | 1.00 | 75.77 | B |
| ATOM | 4118 | NH1 | ARG | B | 98 | −40.577 | 29.583 | 58.437 | 1.00 | 76.70 | B |
| ATOM | 4119 | NH2 | ARG | B | 98 | −42.060 | 31.280 | 57.954 | 1.00 | 76.27 | B |
| ATOM | 4120 | C | ARG | B | 98 | −37.736 | 28.687 | 64.410 | 1.00 | 56.53 | B |
| ATOM | 4121 | O | ARG | B | 98 | −37.181 | 27.609 | 64.256 | 1.00 | 55.93 | B |
| ATOM | 4122 | N | TYR | B | 99 | −38.044 | 29.173 | 65.600 | 1.00 | 55.14 | B |
| ATOM | 4123 | CA | TYR | B | 99 | −37.766 | 28.417 | 66.802 | 1.00 | 55.11 | B |
| ATOM | 4124 | CB | TYR | B | 99 | −36.775 | 29.159 | 67.694 | 1.00 | 55.97 | B |
| ATOM | 4125 | CG | TYR | B | 99 | −35.427 | 29.333 | 67.061 | 1.00 | 57.53 | B |
| ATOM | 4126 | CD1 | TYR | B | 99 | −35.188 | 30.366 | 66.158 | 1.00 | 56.63 | B |
| ATOM | 4127 | CE1 | TYR | B | 99 | −33.970 | 30.483 | 65.516 | 1.00 | 57.52 | B |
| ATOM | 4128 | CD2 | TYR | B | 99 | −34.406 | 28.422 | 67.310 | 1.00 | 58.11 | B |
| ATOM | 4129 | CE2 | TYR | B | 99 | −33.190 | 28.533 | 66.673 | 1.00 | 58.51 | B |
| ATOM | 4130 | CZ | TYR | B | 99 | −32.976 | 29.562 | 65.778 | 1.00 | 58.43 | B |
| ATOM | 4131 | OH | TYR | B | 99 | −31.761 | 29.664 | 65.137 | 1.00 | 61.82 | B |
| ATOM | 4132 | C | TYR | B | 99 | −39.051 | 28.208 | 67.567 | 1.00 | 55.71 | B |
| ATOM | 4133 | O | TYR | B | 99 | −40.014 | 28.949 | 67.389 | 1.00 | 56.08 | B |
| ATOM | 4134 | N | ILE | B | 100 | −39.061 | 27.190 | 68.415 | 1.00 | 55.07 | B |
| ATOM | 4135 | CA | ILE | B | 100 | −40.217 | 26.904 | 69.235 | 1.00 | 54.83 | B |
| ATOM | 4136 | CB | ILE | B | 100 | −40.813 | 25.528 | 68.909 | 1.00 | 54.15 | B |
| ATOM | 4137 | CG2 | ILE | B | 100 | −42.158 | 25.394 | 69.558 | 1.00 | 53.86 | B |
| ATOM | 4138 | CG1 | ILE | B | 100 | −40.939 | 25.340 | 67.393 | 1.00 | 54.26 | B |
| ATOM | 4139 | CD1 | ILE | B | 100 | −41.871 | 26.288 | 66.703 | 1.00 | 53.39 | B |
| ATOM | 4140 | C | ILE | B | 100 | −39.698 | 26.887 | 70.668 | 1.00 | 56.08 | B |
| ATOM | 4141 | O | ILE | B | 100 | −38.591 | 26.426 | 70.924 | 1.00 | 57.33 | B |
| ATOM | 4142 | N | ALA | B | 101 | −40.477 | 27.414 | 71.598 | 1.00 | 56.59 | B |
| ATOM | 4143 | CA | ALA | B | 101 | −40.063 | 27.416 | 72.985 | 1.00 | 56.80 | B |
| ATOM | 4144 | CB | ALA | B | 101 | −40.786 | 28.509 | 73.738 | 1.00 | 55.74 | B |
| ATOM | 4145 | C | ALA | B | 101 | −40.427 | 26.038 | 73.531 | 1.00 | 57.70 | B |
| ATOM | 4146 | O | ALA | B | 101 | −41.574 | 25.608 | 73.442 | 1.00 | 57.75 | B |
| ATOM | 4147 | N | ARG | B | 102 | −39.440 | 25.346 | 74.086 | 1.00 | 59.37 | B |
| ATOM | 4148 | CA | ARG | B | 102 | −39.649 | 24.009 | 74.609 | 1.00 | 60.85 | B |
| ATOM | 4149 | CB | ARG | B | 102 | −39.038 | 22.980 | 73.657 | 1.00 | 62.63 | B |
| ATOM | 4150 | CG | ARG | B | 102 | −40.027 | 22.243 | 72.778 | 1.00 | 62.84 | B |
| ATOM | 4151 | CD | ARG | B | 102 | −40.588 | 21.004 | 73.463 | 1.00 | 62.10 | B |
| ATOM | 4152 | NE | ARG | B | 102 | −40.506 | 19.847 | 72.571 | 1.00 | 63.69 | B |
| ATOM | 4153 | CZ | ARG | B | 102 | −41.034 | 18.656 | 72.836 | 1.00 | 64.27 | B |
| ATOM | 4154 | NH1 | ARG | B | 102 | −41.694 | 18.454 | 73.969 | 1.00 | 64.47 | B |
| ATOM | 4155 | NH2 | ARG | B | 102 | −40.893 | 17.660 | 71.973 | 1.00 | 64.12 | B |
| ATOM | 4156 | C | ARG | B | 102 | −39.024 | 23.828 | 75.967 | 1.00 | 61.59 | B |
| ATOM | 4157 | O | ARG | B | 102 | −37.814 | 24.030 | 76.131 | 1.00 | 61.08 | B |
| ATOM | 4158 | N | ASN | B | 103 | −39.848 | 23.427 | 76.930 | 1.00 | 63.03 | B |
| ATOM | 4159 | CA | ASN | B | 103 | −39.374 | 23.190 | 78.284 | 1.00 | 65.88 | B |
| ATOM | 4160 | CB | ASN | B | 103 | −38.371 | 22.030 | 78.278 | 1.00 | 68.91 | B |
| ATOM | 4161 | CG | ASN | B | 103 | −37.842 | 21.707 | 79.660 | 1.00 | 73.34 | B |
| ATOM | 4162 | OD1 | ASN | B | 103 | −38.601 | 21.682 | 80.641 | 1.00 | 75.48 | B |
| ATOM | 4163 | ND2 | ASN | B | 103 | −36.534 | 21.441 | 79.748 | 1.00 | 74.19 | B |
| ATOM | 4164 | C | ASN | B | 103 | −38.734 | 24.449 | 78.874 | 1.00 | 65.86 | B |
| ATOM | 4165 | O | ASN | B | 103 | −38.342 | 24.467 | 80.046 | 1.00 | 66.33 | B |
| ATOM | 4166 | N | GLY | B | 104 | −38.629 | 25.494 | 78.052 | 1.00 | 64.70 | B |
| ATOM | 4167 | CA | GLY | B | 104 | −38.061 | 26.741 | 78.508 | 1.00 | 60.47 | B |
| ATOM | 4168 | C | GLY | B | 104 | −37.134 | 27.445 | 77.554 | 1.00 | 58.07 | B |
| ATOM | 4169 | O | GLY | B | 104 | −36.759 | 28.586 | 77.812 | 1.00 | 59.48 | B |
| ATOM | 4170 | N | THR | B | 105 | −36.771 | 26.808 | 76.449 | 1.00 | 56.34 | B |
| ATOM | 4171 | CA | THR | B | 105 | −35.845 | 27.445 | 75.511 | 1.00 | 55.26 | B |
| ATOM | 4172 | CB | THR | B | 105 | −34.413 | 26.946 | 75.759 | 1.00 | 56.56 | B |
| ATOM | 4173 | OG1 | THR | B | 105 | −34.011 | 26.101 | 74.674 | 1.00 | 57.15 | B |
| ATOM | 4174 | CG2 | THR | B | 105 | −34.344 | 26.143 | 77.065 | 1.00 | 56.81 | B |
| ATOM | 4175 | C | THR | B | 105 | −36.153 | 27.234 | 74.029 | 1.00 | 53.29 | B |
| ATOM | 4176 | O | THR | B | 105 | −36.884 | 26.316 | 73.662 | 1.00 | 53.19 | B |
| ATOM | 4177 | N | PRO | B | 106 | −35.570 | 28.073 | 73.154 | 1.00 | 51.71 | B |
| ATOM | 4178 | CD | PRO | B | 106 | −34.576 | 29.126 | 73.405 | 1.00 | 50.34 | B |
| ATOM | 4179 | CA | PRO | B | 106 | −35.810 | 27.937 | 71.714 | 1.00 | 51.06 | B |
| ATOM | 4180 | CB | PRO | B | 106 | −35.004 | 29.088 | 71.102 | 1.00 | 49.42 | B |
| ATOM | 4181 | CG | PRO | B | 106 | −34.787 | 30.029 | 72.219 | 1.00 | 49.15 | B |
| ATOM | 4182 | C | PRO | B | 106 | −35.283 | 26.588 | 71.231 | 1.00 | 50.74 | B |
| ATOM | 4183 | O | PRO | B | 106 | −34.341 | 26.036 | 71.803 | 1.00 | 50.71 | B |
| ATOM | 4184 | N | VAL | B | 107 | −35.894 | 26.063 | 70.177 | 1.00 | 50.45 | B |
| ATOM | 4185 | CA | VAL | B | 107 | −35.489 | 24.789 | 69.596 | 1.00 | 50.11 | B |
| ATOM | 4186 | CB | VAL | B | 107 | −36.377 | 23.611 | 70.064 | 1.00 | 49.44 | B |
| ATOM | 4187 | CG1 | VAL | B | 107 | −35.905 | 22.322 | 69.420 | 1.00 | 46.91 | B |
| ATOM | 4188 | CG2 | VAL | B | 107 | −36.353 | 23.493 | 71.574 | 1.00 | 48.19 | B |
| ATOM | 4189 | C | VAL | B | 107 | −35.726 | 24.965 | 68.130 | 1.00 | 50.04 | B |
| ATOM | 4190 | O | VAL | B | 107 | −36.864 | 25.183 | 67.736 | 1.00 | 51.54 | B |
| ATOM | 4191 | N | LEU | B | 108 | −34.679 | 24.891 | 67.319 | 1.00 | 50.33 | B |
| ATOM | 4192 | CA | LEU | B | 108 | −34.876 | 25.049 | 65.879 | 1.00 | 51.44 | B |
| ATOM | 4193 | CB | LEU | B | 108 | −33.598 | 24.731 | 65.088 | 1.00 | 48.92 | B |
| ATOM | 4194 | CG | LEU | B | 108 | −33.802 | 24.943 | 63.586 | 1.00 | 46.83 | B |
| ATOM | 4195 | CD1 | LEU | B | 108 | −34.009 | 26.418 | 63.337 | 1.00 | 51.14 | B |
| ATOM | 4196 | CD2 | LEU | B | 108 | −32.626 | 24.478 | 62.799 | 1.00 | 46.89 | B |
| ATOM | 4197 | C | LEU | B | 108 | −36.010 | 24.144 | 65.385 | 1.00 | 52.87 | B |
| ATOM | 4198 | O | LEU | B | 108 | −36.162 | 22.990 | 65.803 | 1.00 | 54.41 | B |
| ATOM | 4199 | N | LEU | B | 109 | −36.817 | 24.684 | 64.494 | 1.00 | 54.05 | B |
| ATOM | 4200 | CA | LEU | B | 109 | −37.926 | 23.942 | 63.950 | 1.00 | 56.76 | B |
| ATOM | 4201 | CB | LEU | B | 109 | −39.127 | 24.868 | 63.821 | 1.00 | 58.66 | B |
| ATOM | 4202 | CG | LEU | B | 109 | −40.340 | 24.371 | 63.036 | 1.00 | 60.75 | B |
| ATOM | 4203 | CD1 | LEU | B | 109 | −41.146 | 23.352 | 63.868 | 1.00 | 59.45 | B |
| ATOM | 4204 | CD2 | LEU | B | 109 | −41.195 | 25.587 | 62.671 | 1.00 | 60.97 | B |
| ATOM | 4205 | C | LEU | B | 109 | −37.527 | 23.440 | 62.578 | 1.00 | 58.26 | B |
| ATOM | 4206 | O | LEU | B | 109 | −36.800 | 24.123 | 61.859 | 1.00 | 59.58 | B |
| ATOM | 4207 | N | PRO | B | 110 | −37.968 | 22.227 | 62.208 | 1.00 | 58.98 | B |
| ATOM | 4208 | CD | PRO | B | 110 | −38.530 | 21.220 | 63.122 | 1.00 | 59.45 | B |
| ATOM | 4209 | CA | PRO | B | 110 | −37.673 | 21.621 | 60.905 | 1.00 | 59.86 | B |
| ATOM | 4210 | CB | PRO | B | 110 | −38.424 | 20.297 | 60.977 | 1.00 | 58.94 | B |
| ATOM | 4211 | CG | PRO | B | 110 | −38.267 | 19.922 | 62.380 | 1.00 | 58.49 | B |
| ATOM | 4212 | C | PRO | B | 110 | −38.213 | 22.523 | 59.793 | 1.00 | 61.04 | B |
| ATOM | 4213 | O | PRO | B | 110 | −39.430 | 22.726 | 59.700 | 1.00 | 60.55 | B |
| ATOM | 4214 | N | SER | B | 111 | −37.323 | 23.059 | 58.957 | 1.00 | 62.75 | B |
| ATOM | 4215 | CA | SER | B | 111 | −37.746 | 23.961 | 57.879 | 1.00 | 65.70 | B |
| ATOM | 4216 | CB | SER | B | 111 | −36.570 | 24.332 | 56.974 | 1.00 | 65.14 | B |
| ATOM | 4217 | OG | SER | B | 111 | −36.143 | 23.213 | 56.221 | 1.00 | 67.10 | B |
| ATOM | 4218 | C | SER | B | 111 | −38.859 | 23.350 | 57.039 | 1.00 | 66.86 | B |
| ATOM | 4219 | O | SER | B | 111 | −39.940 | 23.921 | 56.919 | 1.00 | 67.57 | B |
| ATOM | 4220 | N | ASN | B | 112 | −38.600 | 22.187 | 56.463 | 1.00 | 68.20 | B |
| ATOM | 4221 | CA | ASN | B | 112 | −39.609 | 21.529 | 55.654 | 1.00 | 70.73 | B |
| ATOM | 4222 | CB | ASN | B | 112 | −39.187 | 21.527 | 54.193 | 1.00 | 72.37 | B |
| ATOM | 4223 | CG | ASN | B | 112 | −37.712 | 21.277 | 54.029 | 1.00 | 74.53 | B |
| ATOM | 4224 | OD1 | ASN | B | 112 | −37.139 | 20.405 | 54.693 | 1.00 | 73.47 | B |
| ATOM | 4225 | ND2 | ASN | B | 112 | −37.079 | 22.041 | 53.139 | 1.00 | 75.26 | B |
| ATOM | 4226 | C | ASN | B | 112 | −39.855 | 20.101 | 56.123 | 1.00 | 71.31 | B |
| ATOM | 4227 | O | ASN | B | 112 | −38.911 | 19.345 | 56.371 | 1.00 | 69.79 | B |
| ATOM | 4228 | N | PRO | B | 113 | −41.142 | 19.720 | 56.260 | 1.00 | 72.24 | B |
| ATOM | 4229 | CD | PRO | B | 113 | −42.313 | 20.609 | 56.099 | 1.00 | 71.27 | B |
| ATOM | 4230 | CA | PRO | B | 113 | −41.563 | 18.384 | 56.700 | 1.00 | 72.55 | B |
| ATOM | 4231 | CB | PRO | B | 113 | −43.078 | 18.434 | 56.499 | 1.00 | 73.02 | B |
| ATOM | 4232 | CG | PRO | B | 113 | −43.397 | 19.874 | 56.851 | 1.00 | 71.22 | B |
| ATOM | 4233 | C | PRO | B | 113 | −40.893 | 17.224 | 55.945 | 1.00 | 71.70 | B |
| ATOM | 4234 | O | PRO | B | 113 | −41.374 | 16.798 | 54.893 | 1.00 | 71.69 | B |
| ATOM | 4235 | N | ILE | B | 114 | −39.783 | 16.732 | 56.498 | 1.00 | 70.19 | B |
| ATOM | 4236 | CA | ILE | B | 114 | −39.010 | 15.627 | 55.921 | 1.00 | 69.47 | B |
| ATOM | 4237 | CB | ILE | B | 114 | −38.664 | 15.895 | 54.416 | 1.00 | 70.14 | B |
| ATOM | 4238 | CG2 | ILE | B | 114 | −37.696 | 17.057 | 54.291 | 1.00 | 68.37 | B |
| ATOM | 4239 | CG1 | ILE | B | 114 | −38.119 | 14.617 | 53.756 | 1.00 | 71.04 | B |
| ATOM | 4240 | CD1 | ILE | B | 114 | −36.711 | 14.202 | 54.178 | 1.00 | 72.05 | B |
| ATOM | 4241 | C | ILE | B | 114 | −37.738 | 15.495 | 56.775 | 1.00 | 69.23 | B |
| ATOM | 4242 | O | ILE | B | 114 | −37.133 | 14.415 | 56.897 | 1.00 | 68.60 | B |
| ATOM | 4243 | N | ASP | B | 115 | −37.334 | 16.616 | 57.361 | 1.00 | 67.87 | B |
| ATOM | 4244 | CA | ASP | B | 115 | −36.193 | 16.629 | 58.262 | 1.00 | 66.35 | B |
| ATOM | 4245 | CB | ASP | B | 115 | −35.539 | 18.014 | 58.302 | 1.00 | 69.18 | B |
| ATOM | 4246 | CG | ASP | B | 115 | −35.675 | 18.765 | 56.988 | 1.00 | 73.26 | B |
| ATOM | 4247 | OD1 | ASP | B | 115 | −35.177 | 18.266 | 55.956 | 1.00 | 75.72 | B |
| ATOM | 4248 | OD2 | ASP | B | 115 | −36.286 | 19.860 | 56.985 | 1.00 | 75.84 | B |
| ATOM | 4249 | C | ASP | B | 115 | −36.940 | 16.372 | 59.567 | 1.00 | 63.74 | B |
| ATOM | 4250 | O | ASP | B | 115 | −36.362 | 16.250 | 60.644 | 1.00 | 61.75 | B |
| ATOM | 4251 | N | LEU | B | 116 | −38.259 | 16.292 | 59.422 | 1.00 | 61.45 | B |
| ATOM | 4252 | CA | LEU | B | 116 | −39.172 | 16.071 | 60.524 | 1.00 | 60.15 | B |
| ATOM | 4253 | CB | LEU | B | 116 | −40.594 | 16.290 | 60.053 | 1.00 | 59.21 | B |
| ATOM | 4254 | CG | LEU | B | 116 | −41.558 | 16.755 | 61.125 | 1.00 | 58.50 | B |
| ATOM | 4255 | CD1 | LEU | B | 116 | −40.989 | 18.000 | 61.804 | 1.00 | 58.10 | B |
| ATOM | 4256 | CD2 | LEU | B | 116 | −42.904 | 17.056 | 60.470 | 1.00 | 58.31 | B |
| ATOM | 4257 | C | LEU | B | 116 | −39.053 | 14.670 | 61.060 | 1.00 | 60.37 | B |
| ATOM | 4258 | O | LEU | B | 116 | −39.739 | 14.316 | 62.011 | 1.00 | 61.93 | B |
| ATOM | 4259 | N | ILE | B | 117 | −38.187 | 13.875 | 60.445 | 1.00 | 59.24 | B |
| ATOM | 4260 | CA | ILE | B | 117 | −37.974 | 12.496 | 60.858 | 1.00 | 58.66 | B |
| ATOM | 4261 | CB | ILE | B | 117 | −38.539 | 11.516 | 59.775 | 1.00 | 58.26 | B |
| ATOM | 4262 | CG2 | ILE | B | 117 | −37.900 | 10.144 | 59.890 | 1.00 | 55.46 | B |
| ATOM | 4263 | CG1 | ILE | B | 117 | −40.059 | 11.422 | 59.929 | 1.00 | 56.85 | B |
| ATOM | 4264 | CD1 | ILE | B | 117 | −40.741 | 10.763 | 58.800 | 1.00 | 56.22 | B |
| ATOM | 4265 | C | ILE | B | 117 | −36.485 | 12.254 | 61.094 | 1.00 | 59.39 | B |
| ATOM | 4266 | O | ILE | B | 117 | −36.103 | 11.361 | 61.859 | 1.00 | 59.59 | B |
| ATOM | 4267 | N | ALA | B | 118 | −35.649 | 13.062 | 60.440 | 1.00 | 59.35 | B |
| ATOM | 4268 | CA | ALA | B | 118 | −34.194 | 12.952 | 60.581 | 1.00 | 59.15 | B |
| ATOM | 4269 | CB | ALA | B | 118 | −33.485 | 13.479 | 59.310 | 1.00 | 55.72 | B |
| ATOM | 4270 | C | ALA | B | 118 | −33.760 | 13.759 | 61.801 | 1.00 | 59.61 | B |
| ATOM | 4271 | O | ALA | B | 118 | −32.850 | 13.363 | 62.531 | 1.00 | 59.64 | B |
| ATOM | 4272 | N | SER | B | 119 | −34.437 | 14.889 | 62.011 | 1.00 | 60.40 | B |
| ATOM | 4273 | CA | SER | B | 119 | −34.164 | 15.799 | 63.122 | 1.00 | 59.93 | B |
| ATOM | 4274 | CB | SER | B | 119 | −34.775 | 17.168 | 62.821 | 1.00 | 59.26 | B |
| ATOM | 4275 | OG | SER | B | 119 | −34.701 | 18.035 | 63.935 | 1.00 | 58.78 | B |
| ATOM | 4276 | C | SER | B | 119 | −34.745 | 15.244 | 64.409 | 1.00 | 60.22 | B |
| ATOM | 4277 | O | SER | B | 119 | −35.536 | 14.307 | 64.378 | 1.00 | 61.78 | B |
| ATOM | 4278 | N | ASN | B | 120 | −34.359 | 15.820 | 65.542 | 1.00 | 60.34 | B |
| ATOM | 4279 | CA | ASN | B | 120 | −34.860 | 15.354 | 66.831 | 1.00 | 59.94 | B |
| ATOM | 4280 | CB | ASN | B | 120 | −33.703 | 15.199 | 67.808 | 1.00 | 63.34 | B |
| ATOM | 4281 | CG | ASN | B | 120 | −33.355 | 13.753 | 68.059 | 1.00 | 67.21 | B |
| ATOM | 4282 | OD1 | ASN | B | 120 | −33.298 | 12.942 | 67.121 | 1.00 | 69.90 | B |
| ATOM | 4283 | ND2 | ASN | B | 120 | −33.112 | 13.413 | 69.325 | 1.00 | 68.09 | B |
| ATOM | 4284 | C | ASN | B | 120 | −35.914 | 16.259 | 67.439 | 1.00 | 58.34 | B |
| ATOM | 4285 | O | ASN | B | 120 | −36.038 | 16.344 | 68.656 | 1.00 | 58.58 | B |
| ATOM | 4286 | N | PHE | B | 121 | −36.678 | 16.942 | 66.599 | 1.00 | 57.13 | B |
| ATOM | 4287 | CA | PHE | B | 121 | −37.700 | 17.822 | 67.118 | 1.00 | 55.57 | B |
| ATOM | 4288 | CB | PHE | B | 121 | −38.306 | 18.676 | 66.015 | 1.00 | 54.20 | B |
| ATOM | 4289 | CG | PHE | B | 121 | −39.247 | 19.725 | 66.527 | 1.00 | 55.97 | B |
| ATOM | 4290 | CD1 | PHE | B | 121 | −38.757 | 20.868 | 67.156 | 1.00 | 55.92 | B |
| ATOM | 4291 | CD2 | PHE | B | 121 | −40.624 | 19.559 | 66.418 | 1.00 | 55.76 | B |
| ATOM | 4292 | CE1 | PHE | B | 121 | −39.623 | 21.821 | 67.666 | 1.00 | 56.15 | B |
| ATOM | 4293 | CE2 | PHE | B | 121 | −41.501 | 20.510 | 66.927 | 1.00 | 55.24 | B |
| ATOM | 4294 | CZ | PHE | B | 121 | −41.003 | 21.641 | 67.552 | 1.00 | 56.13 | B |
| ATOM | 4295 | C | PHE | B | 121 | −38.780 | 16.967 | 67.748 | 1.00 | 56.39 | B |
| ATOM | 4296 | O | PHE | B | 121 | −39.337 | 17.324 | 68.794 | 1.00 | 57.29 | B |
| ATOM | 4297 | N | LEU | B | 122 | −39.068 | 15.828 | 67.113 | 1.00 | 55.25 | B |
| ATOM | 4298 | CA | LEU | B | 122 | −40.096 | 14.906 | 67.611 | 1.00 | 52.02 | B |
| ATOM | 4299 | CB | LEU | B | 122 | −41.026 | 14.463 | 66.473 | 1.00 | 50.77 | B |
| ATOM | 4300 | CG | LEU | B | 122 | −41.772 | 15.527 | 65.672 | 1.00 | 49.52 | B |
| ATOM | 4301 | CD1 | LEU | B | 122 | −42.571 | 14.860 | 64.557 | 1.00 | 50.96 | B |
| ATOM | 4302 | CD2 | LEU | B | 122 | −42.677 | 16.305 | 66.592 | 1.00 | 48.74 | B |
| ATOM | 4303 | C | LEU | B | 122 | −39.481 | 13.672 | 68.246 | 1.00 | 48.85 | B |
| ATOM | 4304 | O | LEU | B | 122 | −38.325 | 13.350 | 68.023 | 1.00 | 50.16 | B |
| ATOM | 4305 | N | SER | B | 123 | −40.268 | 12.982 | 69.045 | 1.00 | 45.68 | B |
| ATOM | 4306 | CA | SER | B | 123 | −39.806 | 11.766 | 69.682 | 1.00 | 43.50 | B |
| ATOM | 4307 | CB | SER | B | 123 | −40.684 | 11.481 | 70.886 | 1.00 | 44.36 | B |
| ATOM | 4308 | OG | SER | B | 123 | −42.052 | 11.438 | 70.500 | 1.00 | 43.25 | B |
| ATOM | 4309 | C | SER | B | 123 | −39.901 | 10.577 | 68.714 | 1.00 | 43.53 | B |
| ATOM | 4310 | O | SER | B | 123 | −40.654 | 10.598 | 67.733 | 1.00 | 43.42 | B |
| ATOM | 4311 | N | THR | B | 124 | −39.149 | 9.526 | 68.995 | 1.00 | 41.88 | B |
| ATOM | 4312 | CA | THR | B | 124 | −39.210 | 8.348 | 68.161 | 1.00 | 40.79 | B |
| ATOM | 4313 | CB | THR | B | 124 | −38.356 | 7.251 | 68.727 | 1.00 | 40.01 | B |
| ATOM | 4314 | OG1 | THR | B | 124 | −36.982 | 7.653 | 68.671 | 1.00 | 42.23 | B |
| ATOM | 4315 | CG2 | THR | B | 124 | −38.559 | 5.974 | 67.944 | 1.00 | 40.70 | B |
| ATOM | 4316 | C | THR | B | 124 | −40.651 | 7.895 | 68.173 | 1.00 | 41.74 | B |
| ATOM | 4317 | O | THR | B | 124 | −41.204 | 7.484 | 67.151 | 1.00 | 42.92 | B |
| ATOM | 4318 | N | GLY | B | 125 | −41.266 | 7.974 | 69.345 | 1.00 | 42.17 | B |
| ATOM | 4319 | CA | GLY | B | 125 | −42.655 | 7.582 | 69.452 | 1.00 | 42.46 | B |
| ATOM | 4320 | C | GLY | B | 125 | −43.518 | 8.388 | 68.502 | 1.00 | 43.21 | B |
| ATOM | 4321 | O | GLY | B | 125 | −44.291 | 7.820 | 67.750 | 1.00 | 42.27 | B |
| ATOM | 4322 | N | SER | B | 126 | −43.374 | 9.714 | 68.524 | 1.00 | 44.71 | B |
| ATOM | 4323 | CA | SER | B | 126 | −44.165 | 10.593 | 67.657 | 1.00 | 45.72 | B |
| ATOM | 4324 | CB | SER | B | 126 | −43.935 | 12.060 | 68.029 | 1.00 | 45.32 | B |
| ATOM | 4325 | OG | SER | B | 126 | −45.126 | 12.621 | 68.559 | 1.00 | 44.99 | B |
| ATOM | 4326 | C | SER | B | 126 | −43.905 | 10.389 | 66.161 | 1.00 | 46.45 | B |
| ATOM | 4327 | O | SER | B | 126 | −44.667 | 10.851 | 65.310 | 1.00 | 46.85 | B |
| ATOM | 4328 | N | LYS | B | 127 | −42.823 | 9.702 | 65.834 | 1.00 | 47.00 | B |
| ATOM | 4329 | CA | LYS | B | 127 | −42.538 | 9.435 | 64.439 | 1.00 | 46.06 | B |
| ATOM | 4330 | CB | LYS | B | 127 | −41.031 | 9.341 | 64.212 | 1.00 | 44.66 | B |
| ATOM | 4331 | CG | LYS | B | 127 | −40.261 | 10.539 | 64.725 | 1.00 | 41.90 | B |
| ATOM | 4332 | CD | LYS | B | 127 | −38.819 | 10.472 | 64.270 | 1.00 | 41.97 | B |
| ATOM | 4333 | CE | LYS | B | 127 | −37.979 | 11.533 | 64.937 | 1.00 | 40.71 | B |
| ATOM | 4334 | NZ | LYS | B | 127 | −36.572 | 11.453 | 64.490 | 1.00 | 40.61 | B |
| ATOM | 4335 | C | LYS | B | 127 | −43.220 | 8.114 | 64.089 | 1.00 | 46.40 | B |
| ATOM | 4336 | O | LYS | B | 127 | −43.727 | 7.961 | 62.992 | 1.00 | 46.43 | B |
| ATOM | 4337 | N | LEU | B | 128 | −43.241 | 7.166 | 65.025 | 1.00 | 46.31 | B |
| ATOM | 4338 | CA | LEU | B | 128 | −43.885 | 5.896 | 64.754 | 1.00 | 48.52 | B |
| ATOM | 4339 | CB | LEU | B | 128 | −43.733 | 4.942 | 65.948 | 1.00 | 49.43 | B |
| ATOM | 4340 | CG | LEU | B | 128 | −42.302 | 4.578 | 66.371 | 1.00 | 50.40 | B |
| ATOM | 4341 | CD1 | LEU | B | 128 | −42.339 | 3.554 | 67.517 | 1.00 | 51.73 | B |
| ATOM | 4342 | CD2 | LEU | B | 128 | −41.541 | 4.010 | 65.182 | 1.00 | 48.42 | B |
| ATOM | 4343 | C | LEU | B | 128 | −45.368 | 6.158 | 64.477 | 1.00 | 50.14 | B |
| ATOM | 4344 | O | LEU | B | 128 | −45.914 | 5.723 | 63.457 | 1.00 | 50.75 | B |
| ATOM | 4345 | N | GLN | B | 129 | −46.019 | 6.878 | 65.385 | 1.00 | 50.24 | B |
| ATOM | 4346 | CA | GLN | B | 129 | −47.432 | 7.177 | 65.226 | 1.00 | 49.58 | B |
| ATOM | 4347 | CB | GLN | B | 129 | −47.920 | 8.020 | 66.408 | 1.00 | 47.35 | B |
| ATOM | 4348 | CG | GLN | B | 129 | −49.283 | 8.605 | 66.210 | 1.00 | 47.78 | B |
| ATOM | 4349 | CD | GLN | B | 129 | −50.009 | 8.810 | 67.506 | 1.00 | 50.62 | B |
| ATOM | 4350 | OE1 | GLN | B | 129 | −49.403 | 9.185 | 68.514 | 1.00 | 52.33 | B |
| ATOM | 4351 | NE2 | GLN | B | 129 | −51.323 | 8.575 | 67.495 | 1.00 | 50.83 | B |
| ATOM | 4352 | C | GLN | B | 129 | −47.685 | 7.894 | 63.897 | 1.00 | 48.73 | B |
| ATOM | 4353 | O | GLN | B | 129 | −48.744 | 7.739 | 63.278 | 1.00 | 47.60 | B |
| ATOM | 4354 | N | MSE | B | 130 | −46.691 | 8.657 | 63.459 | 1.00 | 49.12 | B |
| ATOM | 4355 | CA | MSE | B | 130 | −46.781 | 9.423 | 62.220 | 1.00 | 50.37 | B |
| ATOM | 4356 | CB | MSE | B | 130 | −45.724 | 10.529 | 62.203 | 1.00 | 54.28 | B |
| ATOM | 4357 | CG | MSE | B | 130 | −45.865 | 11.498 | 61.028 | 1.00 | 59.21 | B |
| ATOM | 4358 | SE | MSE | B | 130 | −44.782 | 13.120 | 61.184 | 1.00 | 69.51 | B |
| ATOM | 4359 | CE | MSE | B | 130 | −43.231 | 12.581 | 60.142 | 1.00 | 64.77 | B |
| ATOM | 4360 | C | MSE | B | 130 | −46.619 | 8.552 | 60.988 | 1.00 | 48.16 | B |
| ATOM | 4361 | O | MSE | B | 130 | −47.462 | 8.561 | 60.100 | 1.00 | 47.34 | B |
| ATOM | 4362 | N | LEU | B | 131 | −45.518 | 7.819 | 60.931 | 1.00 | 46.50 | B |
| ATOM | 4363 | CA | LEU | B | 131 | −45.251 | 6.920 | 59.823 | 1.00 | 44.99 | B |
| ATOM | 4364 | CB | LEU | B | 131 | −43.913 | 6.233 | 60.045 | 1.00 | 44.56 | B |
| ATOM | 4365 | CG | LEU | B | 131 | −42.710 | 7.174 | 59.956 | 1.00 | 44.43 | B |
| ATOM | 4366 | CD1 | LEU | B | 131 | −41.483 | 6.534 | 60.612 | 1.00 | 44.47 | B |
| ATOM | 4367 | CD2 | LEU | B | 131 | −42.452 | 7.504 | 58.500 | 1.00 | 39.91 | B |
| ATOM | 4368 | C | LEU | B | 131 | −46.358 | 5.868 | 59.657 | 1.00 | 45.34 | B |
| ATOM | 4369 | O | LEU | B | 131 | −46.740 | 5.542 | 58.528 | 1.00 | 45.53 | B |
| ATOM | 4370 | N | LEU | B | 132 | −46.873 | 5.345 | 60.770 | 1.00 | 43.54 | B |
| ATOM | 4371 | CA | LEU | B | 132 | −47.931 | 4.348 | 60.724 | 1.00 | 44.08 | B |
| ATOM | 4372 | CB | LEU | B | 132 | −47.894 | 3.496 | 61.995 | 1.00 | 43.37 | B |
| ATOM | 4373 | CG | LEU | B | 132 | −46.708 | 2.538 | 62.127 | 1.00 | 41.28 | B |
| ATOM | 4374 | CD1 | LEU | B | 132 | −46.794 | 1.739 | 63.407 | 1.00 | 38.78 | B |
| ATOM | 4375 | CD2 | LEU | B | 132 | −46.716 | 1.603 | 60.948 | 1.00 | 40.11 | B |
| ATOM | 4376 | C | LEU | B | 132 | −49.318 | 4.967 | 60.551 | 1.00 | 46.19 | B |
| ATOM | 4377 | O | LEU | B | 132 | −50.318 | 4.271 | 60.430 | 1.00 | 46.70 | B |
| ATOM | 4378 | N | GLU | B | 133 | −49.374 | 6.288 | 60.542 | 1.00 | 50.79 | B |
| ATOM | 4379 | CA | GLU | B | 133 | −50.635 | 7.010 | 60.384 | 1.00 | 53.77 | B |
| ATOM | 4380 | CB | GLU | B | 133 | −50.331 | 8.508 | 60.284 | 1.00 | 54.79 | B |
| ATOM | 4381 | CG | GLU | B | 133 | −51.513 | 9.434 | 60.463 | 1.00 | 57.93 | B |
| ATOM | 4382 | CD | GLU | B | 133 | −52.337 | 9.581 | 59.209 | 1.00 | 58.48 | B |
| ATOM | 4383 | OE1 | GLU | B | 133 | −51.739 | 9.789 | 58.126 | 1.00 | 60.19 | B |
| ATOM | 4384 | OE2 | GLU | B | 133 | −53.581 | 9.504 | 59.315 | 1.00 | 57.97 | B |
| ATOM | 4385 | C | GLU | B | 133 | −51.402 | 6.521 | 59.147 | 1.00 | 55.62 | B |
| ATOM | 4386 | O | GLU | B | 133 | −52.624 | 6.401 | 59.178 | 1.00 | 53.66 | B |
| ATOM | 4387 | N | PRO | B | 134 | −50.684 | 6.223 | 58.043 | 1.00 | 58.51 | B |
| ATOM | 4388 | CD | PRO | B | 134 | −49.231 | 6.395 | 57.844 | 1.00 | 58.83 | B |
| ATOM | 4389 | CA | PRO | B | 134 | −51.309 | 5.748 | 56.807 | 1.00 | 59.55 | B |
| ATOM | 4390 | CB | PRO | B | 134 | −50.118 | 5.536 | 55.885 | 1.00 | 60.96 | B |
| ATOM | 4391 | CG | PRO | B | 134 | −49.130 | 6.566 | 56.367 | 1.00 | 59.67 | B |
| ATOM | 4392 | C | PRO | B | 134 | −52.100 | 4.468 | 57.008 | 1.00 | 60.68 | B |
| ATOM | 4393 | O | PRO | B | 134 | −53.082 | 4.241 | 56.317 | 1.00 | 61.96 | B |
| ATOM | 4394 | N | ILE | B | 135 | −51.661 | 3.634 | 57.947 | 1.00 | 61.48 | B |
| ATOM | 4395 | CA | ILE | B | 135 | −52.342 | 2.377 | 58.252 | 1.00 | 63.44 | B |
| ATOM | 4396 | CB | ILE | B | 135 | −51.347 | 1.275 | 58.686 | 1.00 | 63.22 | B |
| ATOM | 4397 | CG2 | ILE | B | 135 | −52.075 | .187 | 59.457 | 1.00 | 62.14 | B |
| ATOM | 4398 | CG1 | ILE | B | 135 | −50.683 | .652 | 57.465 | 1.00 | 62.49 | B |
| ATOM | 4399 | CD1 | ILE | B | 135 | −51.635 | −.159 | 56.623 | 1.00 | 62.22 | B |
| ATOM | 4400 | C | ILE | B | 135 | −53.384 | 2.522 | 59.363 | 1.00 | 65.78 | B |
| ATOM | 4401 | O | ILE | B | 135 | −54.511 | 2.049 | 59.230 | 1.00 | 66.63 | B |
| ATOM | 4402 | N | LEU | B | 136 | −53.003 | 3.161 | 60.465 | 1.00 | 67.87 | B |
| ATOM | 4403 | CA | LEU | B | 136 | −53.912 | 3.357 | 61.597 | 1.00 | 69.22 | B |
| ATOM | 4404 | CB | LEU | B | 136 | −53.128 | 3.862 | 62.800 | 1.00 | 66.45 | B |
| ATOM | 4405 | CG | LEU | B | 136 | −51.971 | 2.965 | 63.227 | 1.00 | 65.73 | B |
| ATOM | 4406 | CD1 | LEU | B | 136 | −50.869 | 3.774 | 63.882 | 1.00 | 63.81 | B |
| ATOM | 4407 | CD2 | LEU | B | 136 | −52.504 | 1.911 | 64.158 | 1.00 | 65.79 | B |
| ATOM | 4408 | C | LEU | B | 136 | −55.026 | 4.352 | 61.274 | 1.00 | 71.74 | B |
| ATOM | 4409 | O | LEU | B | 136 | −55.949 | 4.528 | 62.062 | 1.00 | 71.75 | B |
| ATOM | 4410 | N | TRP | B | 137 | −54.932 | 5.000 | 60.116 | 1.00 | 75.24 | B |
| ATOM | 4411 | CA | TRP | B | 137 | −55.932 | 5.982 | 59.700 | 1.00 | 77.96 | B |
| ATOM | 4412 | CB | TRP | B | 137 | −55.698 | 7.291 | 60.451 | 1.00 | 78.33 | B |
| ATOM | 4413 | CG | TRP | B | 137 | −56.824 | 7.615 | 61.355 | 1.00 | 80.23 | B |
| ATOM | 4414 | CD2 | TRP | B | 137 | −56.826 | 7.533 | 62.784 | 1.00 | 79.91 | B |
| ATOM | 4415 | CE2 | TRP | B | 137 | −58.128 | 7.878 | 63.221 | 1.00 | 80.43 | B |
| ATOM | 4416 | CE3 | TRP | B | 137 | −55.858 | 7.200 | 63.737 | 1.00 | 78.32 | B |
| ATOM | 4417 | CD1 | TRP | B | 137 | −58.088 | 8.000 | 60.989 | 1.00 | 81.01 | B |
| ATOM | 4418 | NE1 | TRP | B | 137 | −58.876 | 8.159 | 62.106 | 1.00 | 80.92 | B |
| ATOM | 4419 | CZ2 | TRP | B | 137 | −58.485 | 7.898 | 64.576 | 1.00 | 79.11 | B |
| ATOM | 4420 | CZ3 | TRP | B | 137 | −56.213 | 7.221 | 65.081 | 1.00 | 78.38 | B |
| ATOM | 4421 | CH2 | TRP | B | 137 | −57.517 | 7.567 | 65.487 | 1.00 | 78.30 | B |
| ATOM | 4422 | C | TRP | B | 137 | −55.948 | 6.246 | 58.187 | 1.00 | 79.13 | B |
| ATOM | 4423 | O | TRP | B | 137 | −56.816 | 5.748 | 57.463 | 1.00 | 80.03 | B |
| ATOM | 4424 | N | SER | B | 148 | −67.694 | 17.050 | 61.331 | 1.00 | 88.37 | B |
| ATOM | 4425 | CA | SER | B | 148 | −68.851 | 17.909 | 61.562 | 1.00 | 88.37 | B |
| ATOM | 4426 | CB | SER | B | 148 | −69.868 | 17.188 | 62.451 | 1.00 | 89.18 | B |
| ATOM | 4427 | OG | SER | B | 148 | −70.239 | 15.938 | 61.892 | 1.00 | 89.82 | B |
| ATOM | 4428 | C | SER | B | 148 | −68.377 | 19.185 | 62.248 | 1.00 | 87.84 | B |
| ATOM | 4429 | O | SER | B | 148 | −68.690 | 20.305 | 61.828 | 1.00 | 87.75 | B |
| ATOM | 4430 | N | HIS | B | 149 | −67.616 | 18.989 | 63.314 | .00 | 86.43 | B |
| ATOM | 4431 | CA | HIS | B | 149 | −67.048 | 20.074 | 64.093 | .00 | 85.11 | B |
| ATOM | 4432 | CB | HIS | B | 149 | −67.781 | 20.216 | 65.421 | 1.00 | 86.23 | B |
| ATOM | 4433 | CG | HIS | B | 149 | −69.143 | 20.821 | 65.299 | 1.00 | 89.19 | B |
| ATOM | 4434 | CD2 | HIS | B | 149 | −69.723 | 21.851 | 65.961 | 1.00 | 89.88 | B |
| ATOM | 4435 | ND1 | HIS | B | 149 | −70.092 | 20.350 | 64.417 | 1.00 | 90.12 | B |
| ATOM | 4436 | CE1 | HIS | B | 149 | −71.198 | 21.062 | 64.543 | 1.00 | 90.78 | B |
| ATOM | 4437 | NE2 | HIS | B | 149 | −71.001 | 21.979 | 65.474 | 1.00 | 90.68 | B |
| ATOM | 4438 | C | HIS | B | 149 | −65.613 | 19.673 | 64.349 | .00 | 83.19 | B |
| ATOM | 4439 | O | HIS | B | 149 | −64.726 | 19.954 | 63.545 | .00 | 83.07 | B |
| ATOM | 4440 | N | GLU | B | 150 | −65.402 | 18.997 | 65.472 | 1.00 | 80.85 | B |
| ATOM | 4441 | CA | GLU | B | 150 | −64.080 | 18.521 | 65.852 | 1.00 | 78.35 | B |
| ATOM | 4442 | CB | GLU | B | 150 | −63.727 | 17.264 | 65.053 | 1.00 | 80.57 | B |
| ATOM | 4443 | CG | GLU | B | 150 | −62.298 | 16.800 | 65.242 | 1.00 | 83.97 | B |
| ATOM | 4444 | CD | GLU | B | 150 | −61.925 | 15.713 | 64.264 | 1.00 | 85.84 | B |
| ATOM | 4445 | OE1 | GLU | B | 150 | −62.377 | 15.809 | 63.101 | 1.00 | 86.67 | B |
| ATOM | 4446 | OE2 | GLU | B | 150 | −61.182 | 14.778 | 64.647 | 1.00 | 86.58 | B |
| ATOM | 4447 | C | GLU | B | 150 | −63.002 | 19.580 | 65.659 | 1.00 | 74.63 | B |
| ATOM | 4448 | O | GLU | B | 150 | −62.621 | 19.936 | 64.541 | 1.00 | 73.87 | B |
| ATOM | 4449 | N | SER | B | 151 | −62.501 | 20.062 | 66.781 | 1.00 | 70.35 | B |
| ATOM | 4450 | CA | SER | B | 151 | −61.478 | 21.087 | 66.804 | 1.00 | 66.66 | B |
| ATOM | 4451 | CB | SER | B | 151 | −61.190 | 21.471 | 68.253 | 1.00 | 66.53 | B |
| ATOM | 4452 | OG | SER | B | 151 | −60.666 | 20.363 | 68.969 | 1.00 | 63.90 | B |
| ATOM | 4453 | C | SER | B | 151 | −60.169 | 20.688 | 66.133 | 1.00 | 63.87 | B |
| ATOM | 4454 | O | SER | B | 151 | −59.844 | 19.507 | 66.027 | 1.00 | 61.67 | B |
| ATOM | 4455 | N | VAL | B | 152 | −59.430 | 21.701 | 65.686 | 1.00 | 61.09 | B |
| ATOM | 4456 | CA | VAL | B | 152 | −58.133 | 21.511 | 65.064 | 1.00 | 58.60 | B |
| ATOM | 4457 | CB | VAL | B | 152 | −57.356 | 22.842 | 65.015 | 1.00 | 58.15 | B |
| ATOM | 4458 | CG1 | VAL | B | 152 | −55.907 | 22.600 | 64.591 | 1.00 | 56.28 | B |
| ATOM | 4459 | CG2 | VAL | B | 152 | −58.062 | 23.816 | 64.086 | 1.00 | 56.77 | B |
| ATOM | 4460 | C | VAL | B | 152 | −57.369 | 20.566 | 65.972 | 1.00 | 58.33 | B |
| ATOM | 4461 | O | VAL | B | 152 | −56.701 | 19.639 | 65.517 | 1.00 | 59.59 | B |
| ATOM | 4462 | N | SER | B | 153 | −57.495 | 20.820 | 67.270 | 1.00 | 56.75 | B |
| ATOM | 4463 | CA | SER | B | 153 | −56.829 | 20.057 | 68.306 | 1.00 | 55.49 | B |
| ATOM | 4464 | CB | SER | B | 153 | −57.094 | 20.714 | 69.650 | 1.00 | 55.70 | B |
| ATOM | 4465 | OG | SER | B | 153 | −56.298 | 20.136 | 70.669 | 1.00 | 57.28 | B |
| ATOM | 4466 | C | SER | B | 153 | −57.240 | 18.592 | 68.370 | 1.00 | 55.65 | B |
| ATOM | 4467 | O | SER | B | 153 | −56.392 | 17.710 | 68.470 | 1.00 | 56.00 | B |
| ATOM | 4468 | N | GLY | B | 154 | −58.537 | 18.324 | 68.325 | 1.00 | 55.60 | B |
| ATOM | 4469 | CA | GLY | B | 154 | −58.991 | 16.940 | 68.393 | 1.00 | 55.26 | B |
| ATOM | 4470 | C | GLY | B | 154 | −58.516 | 16.113 | 67.213 | 1.00 | 55.18 | B |
| ATOM | 4471 | O | GLY | B | 154 | −57.980 | 15.013 | 67.369 | 1.00 | 52.89 | B |
| ATOM | 4472 | N | PHE | B | 155 | −58.740 | 16.654 | 66.022 | 1.00 | 55.24 | B |
| ATOM | 4473 | CA | PHE | B | 155 | −58.332 | 16.012 | 64.791 | 1.00 | 54.90 | B |
| ATOM | 4474 | CB | PHE | B | 155 | −58.529 | 16.992 | 63.643 | 1.00 | 56.11 | B |
| ATOM | 4475 | CG | PHE | B | 155 | −58.030 | 16.511 | 62.313 | 1.00 | 58.44 | B |
| ATOM | 4476 | CD1 | PHE | B | 155 | −58.924 | 16.328 | 61.260 | 1.00 | 58.41 | B |
| ATOM | 4477 | CD2 | PHE | B | 155 | −56.662 | 16.365 | 62.071 | 1.00 | 58.14 | B |
| ATOM | 4478 | CE1 | PHE | B | 155 | −58.469 | 16.024 | 59.990 | 1.00 | 59.90 | B |
| ATOM | 4479 | CE2 | PHE | B | 155 | −56.196 | 16.060 | 60.806 | 1.00 | 59.80 | B |
| ATOM | 4480 | CZ | PHE | B | 155 | −57.100 | 15.891 | 59.758 | 1.00 | 61.08 | B |
| ATOM | 4481 | C | PHE | B | 155 | −56.864 | 15.632 | 64.929 | 1.00 | 55.11 | B |
| ATOM | 4482 | O | PHE | B | 155 | −56.537 | 14.451 | 65.086 | 1.00 | 55.69 | B |
| ATOM | 4483 | N | PHE | B | 156 | −55.983 | 16.630 | 64.897 | 1.00 | 53.13 | B |
| ATOM | 4484 | CA | PHE | B | 156 | −54.558 | 16.359 | 64.997 | 1.00 | 51.55 | B |
| ATOM | 4485 | CB | PHE | B | 156 | −53.775 | 17.657 | 65.068 | 1.00 | 47.89 | B |
| ATOM | 4486 | CG | PHE | B | 156 | −53.681 | 18.362 | 63.758 | 1.00 | 46.46 | B |
| ATOM | 4487 | CD1 | PHE | B | 156 | −54.558 | 19.378 | 63.437 | 1.00 | 46.11 | B |
| ATOM | 4488 | CD2 | PHE | B | 156 | −52.731 | 17.987 | 62.829 | 1.00 | 46.64 | B |
| ATOM | 4489 | CE1 | PHE | B | 156 | −54.489 | 20.008 | 62.213 | 1.00 | 46.75 | B |
| ATOM | 4490 | CE2 | PHE | B | 156 | −52.656 | 18.615 | 61.597 | 1.00 | 47.41 | B |
| ATOM | 4491 | CZ | PHE | B | 156 | −53.535 | 19.626 | 61.288 | 1.00 | 46.76 | B |
| ATOM | 4492 | C | PHE | B | 156 | −54.175 | 15.447 | 66.149 | 1.00 | 52.73 | B |
| ATOM | 4493 | O | PHE | B | 156 | −53.267 | 14.637 | 66.026 | 1.00 | 53.30 | B |
| ATOM | 4494 | N | GLN | B | 157 | −54.871 | 15.562 | 67.269 | 1.00 | 55.37 | B |
| ATOM | 4495 | CA | GLN | B | 157 | −54.575 | 14.715 | 68.419 | 1.00 | 57.57 | B |
| ATOM | 4496 | CB | GLN | B | 157 | −55.540 | 15.022 | 69.562 | 1.00 | 57.58 | B |
| ATOM | 4497 | CG | GLN | B | 157 | −55.542 | 13.981 | 70.671 | 1.00 | 58.15 | B |
| ATOM | 4498 | CD | GLN | B | 157 | −54.320 | 14.064 | 71.559 | 1.00 | 60.70 | B |
| ATOM | 4499 | OE1 | GLN | B | 157 | −54.335 | 14.703 | 72.618 | 1.00 | 63.80 | B |
| ATOM | 4500 | NE2 | GLN | B | 157 | −53.247 | 13.432 | 71.129 | 1.00 | 60.63 | B |
| ATOM | 4501 | C | GLN | B | 157 | −54.686 | 13.237 | 68.058 | 1.00 | 59.49 | B |
| ATOM | 4502 | O | GLN | B | 157 | −53.701 | 12.505 | 68.120 | 1.00 | 59.60 | B |
| ATOM | 4503 | N | ARG | B | 158 | −55.891 | 12.814 | 67.674 | 1.00 | 62.21 | B |
| ATOM | 4504 | CA | ARG | B | 158 | −56.165 | 11.421 | 67.327 | 1.00 | 63.69 | B |
| ATOM | 4505 | CB | ARG | B | 158 | −57.652 | 11.226 | 66.935 | 1.00 | 66.46 | B |
| ATOM | 4506 | CG | ARG | B | 158 | −58.143 | 12.076 | 65.744 | 1.00 | 70.76 | B |
| ATOM | 4507 | CD | ARG | B | 158 | −59.549 | 11.669 | 65.210 | 1.00 | 73.20 | B |
| ATOM | 4508 | NE | ARG | B | 158 | −60.607 | 11.626 | 66.232 | 1.00 | 75.92 | B |
| ATOM | 4509 | CZ | ARG | B | 158 | −60.981 | 12.644 | 67.013 | 1.00 | 76.12 | B |
| ATOM | 4510 | NH1 | ARG | B | 158 | −60.394 | 13.832 | 66.920 | 1.00 | 75.65 | B |
| ATOM | 4511 | NH2 | ARG | B | 158 | −61.955 | 12.468 | 67.900 | 1.00 | 76.52 | B |
| ATOM | 4512 | C | ARG | B | 158 | −55.274 | 10.891 | 66.215 | 1.00 | 63.73 | B |
| ATOM | 4513 | O | ARG | B | 158 | −54.909 | 9.714 | 66.221 | 1.00 | 65.64 | B |
| ATOM | 4514 | N | HIS | B | 159 | −54.907 | 11.748 | 65.270 | 1.00 | 61.67 | B |
| ATOM | 4515 | CA | HIS | B | 159 | −54.073 | 11.308 | 64.163 | 1.00 | 59.72 | B |
| ATOM | 4516 | CB | HIS | B | 159 | −54.295 | 12.211 | 62.953 | 1.00 | 62.16 | B |
| ATOM | 4517 | CG | HIS | B | 159 | −55.385 | 11.744 | 62.043 | 1.00 | 63.34 | B |
| ATOM | 4518 | CD2 | HIS | B | 159 | −56.731 | 11.859 | 62.131 | 1.00 | 64.44 | B |
| ATOM | 4519 | ND1 | HIS | B | 159 | −55.132 | 11.065 | 60.872 | 1.00 | 63.30 | B |
| ATOM | 4520 | CE1 | HIS | B | 159 | −56.276 | 10.784 | 60.276 | 1.00 | 64.04 | B |
| ATOM | 4521 | NE2 | HIS | B | 159 | −57.262 | 11.254 | 61.020 | 1.00 | 64.51 | B |
| ATOM | 4522 | C | HIS | B | 159 | −52.582 | 11.212 | 64.429 | 1.00 | 57.23 | B |
| ATOM | 4523 | O | HIS | B | 159 | −51.987 | 10.178 | 64.184 | 1.00 | 56.76 | B |
| ATOM | 4524 | N | PHE | B | 160 | −51.975 | 12.283 | 64.923 | 1.00 | 55.46 | B |
| ATOM | 4525 | CA | PHE | B | 160 | −50.538 | 12.278 | 65.145 | 1.00 | 53.26 | B |
| ATOM | 4526 | CB | PHE | B | 160 | −49.909 | 13.429 | 64.375 | 1.00 | 51.52 | B |
| ATOM | 4527 | CG | PHE | B | 160 | −50.131 | 13.343 | 62.902 | 1.00 | 51.93 | B |
| ATOM | 4528 | CD1 | PHE | B | 160 | −51.329 | 13.758 | 62.343 | 1.00 | 50.46 | B |
| ATOM | 4529 | CD2 | PHE | B | 160 | −49.158 | 12.787 | 62.073 | 1.00 | 51.55 | B |
| ATOM | 4530 | CE1 | PHE | B | 160 | −51.556 | 13.614 | 60.981 | 1.00 | 50.01 | B |
| ATOM | 4531 | CE2 | PHE | B | 160 | −49.378 | 12.642 | 60.718 | 1.00 | 49.43 | B |
| ATOM | 4532 | CZ | PHE | B | 160 | −50.578 | 13.054 | 60.170 | 1.00 | 49.37 | B |
| ATOM | 4533 | C | PHE | B | 160 | −50.039 | 12.297 | 66.580 | 1.00 | 52.69 | B |
| ATOM | 4534 | O | PHE | B | 160 | −48.828 | 12.199 | 66.815 | 1.00 | 52.63 | B |
| ATOM | 4535 | N | GLY | B | 161 | −50.944 | 12.427 | 67.539 | 1.00 | 49.86 | B |
| ATOM | 4536 | CA | GLY | B | 161 | −50.492 | 12.434 | 68.908 | 1.00 | 50.49 | B |
| ATOM | 4537 | C | GLY | B | 161 | −50.447 | 13.799 | 69.552 | 1.00 | 51.62 | B |
| ATOM | 4538 | O | GLY | B | 161 | −50.558 | 14.820 | 68.863 | 1.00 | 53.16 | B |
| ATOM | 4539 | N | LYS | B | 162 | −50.266 | 13.805 | 70.875 | 1.00 | 51.31 | B |
| ATOM | 4540 | CA | LYS | B | 162 | −50.246 | 15.032 | 71.671 | 1.00 | 51.96 | B |
| ATOM | 4541 | CB | LYS | B | 162 | −50.426 | 14.698 | 73.153 | 1.00 | 53.43 | B |
| ATOM | 4542 | CG | LYS | B | 162 | −51.409 | 15.620 | 73.845 | 1.00 | 58.08 | B |
| ATOM | 4543 | CD | LYS | B | 162 | −51.584 | 15.272 | 75.315 | 1.00 | 61.48 | B |
| ATOM | 4544 | CE | LYS | B | 162 | −50.358 | 15.663 | 76.131 | 1.00 | 62.51 | B |
| ATOM | 4545 | NZ | LYS | B | 162 | −50.178 | 17.142 | 76.215 | 1.00 | 61.91 | B |
| ATOM | 4546 | C | LYS | B | 162 | −49.016 | 15.918 | 71.499 | 1.00 | 50.31 | B |
| ATOM | 4547 | O | LYS | B | 162 | −49.130 | 17.146 | 71.498 | 1.00 | 48.04 | B |
| ATOM | 4548 | N | GLU | B | 163 | −47.845 | 15.303 | 71.363 | 1.00 | 49.04 | B |
| ATOM | 4549 | CA | GLU | B | 163 | −46.625 | 16.074 | 71.183 | 1.00 | 48.42 | B |
| ATOM | 4550 | CB | GLU | B | 163 | −45.415 | 15.165 | 71.078 | 1.00 | 48.95 | B |
| ATOM | 4551 | CG | GLU | B | 163 | −44.131 | 15.945 | 70.891 | 1.00 | 51.58 | B |
| ATOM | 4552 | CD | GLU | B | 163 | −42.951 | 15.075 | 70.509 | 1.00 | 54.87 | B |
| ATOM | 4553 | OE1 | GLU | B | 163 | −41.801 | 15.573 | 70.572 | 1.00 | 54.99 | B |
| ATOM | 4554 | OE2 | GLU | B | 163 | −43.170 | 13.897 | 70.136 | 1.00 | 55.71 | B |
| ATOM | 4555 | C | GLU | B | 163 | −46.705 | 16.916 | 69.917 | 1.00 | 47.59 | B |
| ATOM | 4556 | O | GLU | B | 163 | −46.292 | 18.066 | 69.889 | 1.00 | 48.49 | B |
| ATOM | 4557 | N | VAL | B | 164 | −47.233 | 16.335 | 68.854 | 1.00 | 47.38 | B |
| ATOM | 4558 | CA | VAL | B | 164 | −47.355 | 17.062 | 67.608 | 1.00 | 45.82 | B |
| ATOM | 4559 | CB | VAL | B | 164 | −47.693 | 16.113 | 66.448 | 1.00 | 41.93 | B |
| ATOM | 4560 | CG1 | VAL | B | 164 | −48.214 | 16.881 | 65.280 | 1.00 | 39.49 | B |
| ATOM | 4561 | CG2 | VAL | B | 164 | −46.440 | 15.386 | 66.024 | 1.00 | 39.69 | B |
| ATOM | 4562 | C | VAL | B | 164 | −48.390 | 18.170 | 67.720 | 1.00 | 47.96 | B |
| ATOM | 4563 | O | VAL | B | 164 | −48.277 | 19.193 | 67.047 | 1.00 | 49.46 | B |
| ATOM | 4564 | N | VAL | B | 165 | −49.400 | 17.986 | 68.564 | 1.00 | 49.38 | B |
| ATOM | 4565 | CA | VAL | B | 165 | −50.400 | 19.040 | 68.734 | 1.00 | 50.42 | B |
| ATOM | 4566 | CB | VAL | B | 165 | −51.647 | 18.533 | 69.458 | 1.00 | 50.30 | B |
| ATOM | 4567 | CG1 | VAL | B | 165 | −52.508 | 19.718 | 69.883 | 1.00 | 49.85 | B |
| ATOM | 4568 | CG2 | VAL | B | 165 | −52.423 | 17.609 | 68.550 | 1.00 | 49.50 | B |
| ATOM | 4569 | C | VAL | B | 165 | −49.801 | 20.181 | 69.560 | 1.00 | 50.20 | B |
| ATOM | 4570 | O | VAL | B | 165 | −49.904 | 21.351 | 69.199 | 1.00 | 49.20 | B |
| ATOM | 4571 | N | ASP | B | 166 | −49.153 | 19.811 | 70.659 | 1.00 | 51.16 | B |
| ATOM | 4572 | CA | ASP | B | 166 | −48.523 | 20.755 | 71.569 | 1.00 | 51.80 | B |
| ATOM | 4573 | CB | ASP | B | 166 | −48.068 | 20.031 | 72.824 | 1.00 | 52.55 | B |
| ATOM | 4574 | CG | ASP | B | 166 | −49.225 | 19.509 | 73.635 | 1.00 | 56.40 | B |
| ATOM | 4575 | OD1 | ASP | B | 166 | −48.990 | 18.619 | 74.478 | 1.00 | 58.83 | B |
| ATOM | 4576 | OD2 | ASP | B | 166 | −50.366 | 19.991 | 73.441 | 1.00 | 57.80 | B |
| ATOM | 4577 | C | ASP | B | 166 | −47.350 | 21.544 | 71.017 | 1.00 | 51.96 | B |
| ATOM | 4578 | O | ASP | B | 166 | −47.299 | 22.764 | 71.171 | 1.00 | 54.52 | B |
| ATOM | 4579 | N | TYR | B | 167 | −46.403 | 20.880 | 70.373 | 1.00 | 50.21 | B |
| ATOM | 4580 | CA | TYR | B | 167 | −45.269 | 21.622 | 69.880 | 1.00 | 49.95 | B |
| ATOM | 4581 | CB | TYR | B | 167 | −43.994 | 21.040 | 70.463 | 1.00 | 50.23 | B |
| ATOM | 4582 | CG | TYR | B | 167 | −44.014 | 21.059 | 71.969 | 1.00 | 54.08 | B |
| ATOM | 4583 | CD1 | TYR | B | 167 | −44.471 | 19.959 | 72.690 | 1.00 | 56.26 | B |
| ATOM | 4584 | CE1 | TYR | B | 167 | −44.515 | 19.977 | 74.090 | 1.00 | 57.59 | B |
| ATOM | 4585 | CD2 | TYR | B | 167 | −43.604 | 22.186 | 72.679 | 1.00 | 54.71 | B |
| ATOM | 4586 | CE2 | TYR | B | 167 | −43.645 | 22.220 | 74.075 | 1.00 | 56.82 | B |
| ATOM | 4587 | CZ | TYR | B | 167 | −44.102 | 21.109 | 74.775 | 1.00 | 58.18 | B |
| ATOM | 4588 | OH | TYR | B | 167 | −44.151 | 21.127 | 76.155 | 1.00 | 59.74 | B |
| ATOM | 4589 | C | TYR | B | 167 | −45.143 | 21.798 | 68.381 | 1.00 | 50.74 | B |
| ATOM | 4590 | O | TYR | B | 167 | −44.075 | 22.142 | 67.876 | 1.00 | 52.69 | B |
| ATOM | 4591 | N | LEU | B | 168 | −46.230 | 21.582 | 67.658 | 1.00 | 50.62 | B |
| ATOM | 4592 | CA | LEU | B | 168 | −46.192 | 21.776 | 66.219 | 1.00 | 50.31 | B |
| ATOM | 4593 | CB | LEU | B | 168 | −46.044 | 20.452 | 65.482 | 1.00 | 49.65 | B |
| ATOM | 4594 | CG | LEU | B | 168 | −44.609 | 19.989 | 65.273 | 1.00 | 46.31 | B |
| ATOM | 4595 | CD1 | LEU | B | 168 | −44.573 | 18.742 | 64.381 | 1.00 | 44.34 | B |
| ATOM | 4596 | CD2 | LEU | B | 168 | −43.843 | 21.120 | 64.639 | 1.00 | 43.99 | B |
| ATOM | 4597 | C | LEU | B | 168 | −47.417 | 22.499 | 65.720 | 1.00 | 51.53 | B |
| ATOM | 4598 | O | LEU | B | 168 | −47.334 | 23.678 | 65.396 | 1.00 | 52.69 | B |
| ATOM | 4599 | N | ILE | B | 169 | −48.565 | 21.836 | 65.672 | 1.00 | 52.24 | B |
| ATOM | 4600 | CA | ILE | B | 169 | −49.723 | 22.552 | 65.165 | 1.00 | 54.62 | B |
| ATOM | 4601 | CB | ILE | B | 169 | −50.929 | 21.610 | 64.844 | 1.00 | 54.22 | B |
| ATOM | 4602 | CG2 | ILE | B | 169 | −50.446 | 20.347 | 64.142 | 1.00 | 53.18 | B |
| ATOM | 4603 | CG1 | ILE | B | 169 | −51.719 | 21.315 | 66.104 | 1.00 | 54.21 | B |
| ATOM | 4604 | CD1 | ILE | B | 169 | −52.789 | 22.345 | 66.336 | 1.00 | 55.37 | B |
| ATOM | 4605 | C | ILE | B | 169 | −50.163 | 23.711 | 66.071 | 1.00 | 55.35 | B |
| ATOM | 4606 | O | ILE | B | 169 | −50.714 | 24.688 | 65.574 | 1.00 | 57.42 | B |
| ATOM | 4607 | N | ASP | B | 170 | −49.923 | 23.639 | 67.379 | 1.00 | 55.52 | B |
| ATOM | 4608 | CA | ASP | B | 170 | −50.308 | 24.769 | 68.237 | 1.00 | 55.97 | B |
| ATOM | 4609 | CB | ASP | B | 170 | −50.182 | 24.422 | 69.721 | 1.00 | 59.74 | B |
| ATOM | 4610 | CG | ASP | B | 170 | −50.431 | 25.631 | 70.633 | 1.00 | 62.88 | B |
| ATOM | 4611 | OD1 | ASP | B | 170 | −49.575 | 26.549 | 70.662 | 1.00 | 64.53 | B |
| ATOM | 4612 | OD2 | ASP | B | 170 | −51.482 | 25.663 | 71.319 | 1.00 | 63.91 | B |
| ATOM | 4613 | C | ASP | B | 170 | −49.449 | 26.005 | 67.937 | 1.00 | 54.67 | B |
| ATOM | 4614 | O | ASP | B | 170 | −49.979 | 27.080 | 67.675 | 1.00 | 54.69 | B |
| ATOM | 4615 | N | PRO | B | 171 | −48.113 | 25.874 | 67.980 | 1.00 | 52.77 | B |
| ATOM | 4616 | CD | PRO | B | 171 | −47.261 | 24.711 | 68.270 | 1.00 | 52.82 | B |
| ATOM | 4617 | CA | PRO | B | 171 | −47.294 | 27.049 | 67.689 | 1.00 | 52.38 | B |
| ATOM | 4618 | CB | PRO | B | 171 | −45.887 | 26.468 | 67.610 | 1.00 | 52.79 | B |
| ATOM | 4619 | CG | PRO | B | 171 | −45.943 | 25.351 | 68.582 | 1.00 | 51.66 | B |
| ATOM | 4620 | C | PRO | B | 171 | −47.742 | 27.663 | 66.365 | 1.00 | 52.13 | B |
| ATOM | 4621 | O | PRO | B | 171 | −47.762 | 28.883 | 66.198 | 1.00 | 50.72 | B |
| ATOM | 4622 | N | PHE | B | 172 | −48.113 | 26.796 | 65.429 | 1.00 | 52.42 | B |
| ATOM | 4623 | CA | PHE | B | 172 | −48.569 | 27.237 | 64.119 | 1.00 | 52.68 | B |
| ATOM | 4624 | CB | PHE | B | 172 | −48.855 | 26.032 | 63.229 | 1.00 | 51.20 | B |
| ATOM | 4625 | CG | PHE | B | 172 | −49.412 | 26.394 | 61.881 | 1.00 | 51.11 | B |
| ATOM | 4626 | CD1 | PHE | B | 172 | −50.788 | 26.467 | 61.680 | 1.00 | 50.37 | B |
| ATOM | 4627 | CD2 | PHE | B | 172 | −48.559 | 26.688 | 60.815 | 1.00 | 51.87 | B |
| ATOM | 4628 | CE1 | PHE | B | 172 | −51.317 | 26.827 | 60.449 | 1.00 | 49.74 | B |
| ATOM | 4629 | CE2 | PHE | B | 172 | −49.081 | 27.052 | 59.569 | 1.00 | 53.40 | B |
| ATOM | 4630 | CZ | PHE | B | 172 | −50.467 | 27.122 | 59.390 | 1.00 | 51.93 | B |
| ATOM | 4631 | C | PHE | B | 172 | −49.823 | 28.105 | 64.222 | 1.00 | 53.69 | B |
| ATOM | 4632 | O | PHE | B | 172 | −49.903 | 29.193 | 63.632 | 1.00 | 54.75 | B |
| ATOM | 4633 | N | VAL | B | 173 | −50.811 | 27.625 | 64.962 | 1.00 | 52.10 | B |
| ATOM | 4634 | CA | VAL | B | 173 | −52.031 | 28.390 | 65.111 | 1.00 | 52.35 | B |
| ATOM | 4635 | CB | VAL | B | 173 | −53.101 | 27.538 | 65.830 | 1.00 | 52.49 | B |
| ATOM | 4636 | CG1 | VAL | B | 173 | −54.301 | 28.382 | 66.217 | 1.00 | 53.16 | B |
| ATOM | 4637 | CG2 | VAL | B | 173 | −53.544 | 26.415 | 64.910 | 1.00 | 51.58 | B |
| ATOM | 4638 | C | VAL | B | 173 | −51.707 | 29.682 | 65.877 | 1.00 | 52.71 | B |
| ATOM | 4639 | O | VAL | B | 173 | −52.152 | 30.773 | 65.505 | 1.00 | 53.25 | B |
| ATOM | 4640 | N | ALA | B | 174 | −50.905 | 29.566 | 66.926 | 1.00 | 51.60 | B |
| ATOM | 4641 | CA | ALA | B | 174 | −50.540 | 30.738 | 67.696 | 1.00 | 52.32 | B |
| ATOM | 4642 | CB | ALA | B | 174 | −49.561 | 30.365 | 68.804 | 1.00 | 50.49 | B |
| ATOM | 4643 | C | ALA | B | 174 | −49.912 | 31.768 | 66.763 | 1.00 | 53.59 | B |
| ATOM | 4644 | O | ALA | B | 174 | −50.176 | 32.969 | 66.881 | 1.00 | 54.33 | B |
| ATOM | 4645 | N | GLY | B | 175 | −49.091 | 31.297 | 65.829 | 1.00 | 53.63 | B |
| ATOM | 4646 | CA | GLY | B | 175 | −48.438 | 32.208 | 64.907 | 1.00 | 54.19 | B |
| ATOM | 4647 | C | GLY | B | 175 | −49.328 | 32.706 | 63.781 | 1.00 | 54.98 | B |
| ATOM | 4648 | O | GLY | B | 175 | −48.934 | 33.588 | 63.019 | 1.00 | 54.68 | B |
| ATOM | 4649 | N | THR | B | 176 | −50.533 | 32.163 | 63.662 | 1.00 | 55.15 | B |
| ATOM | 4650 | CA | THR | B | 176 | −51.398 | 32.605 | 62.579 | 1.00 | 56.61 | B |
| ATOM | 4651 | CB | THR | B | 176 | −51.988 | 31.412 | 61.799 | 1.00 | 56.65 | B |
| ATOM | 4652 | OG1 | THR | B | 176 | −50.930 | 30.551 | 61.354 | 1.00 | 55.87 | B |
| ATOM | 4653 | CG2 | THR | B | 176 | −52.762 | 31.916 | 60.579 | 1.00 | 56.14 | B |
| ATOM | 4654 | C | THR | B | 176 | −52.544 | 33.508 | 63.014 | 1.00 | 57.43 | B |
| ATOM | 4655 | O | THR | B | 176 | −53.003 | 34.348 | 62.240 | 1.00 | 59.60 | B |
| ATOM | 4656 | N | CYS | B | 177 | −53.013 | 33.343 | 64.244 | 1.00 | 56.72 | B |
| ATOM | 4657 | CA | CYS | B | 177 | −54.113 | 34.166 | 64.738 | 1.00 | 55.30 | B |
| ATOM | 4658 | CB | CYS | B | 177 | −55.454 | 33.553 | 64.333 | 1.00 | 55.47 | B |
| ATOM | 4659 | SG | CYS | B | 177 | −55.789 | 31.930 | 65.086 | 1.00 | 53.08 | B |
| ATOM | 4660 | C | CYS | B | 177 | −54.053 | 34.302 | 66.254 | 1.00 | 55.09 | B |
| ATOM | 4661 | O | CYS | B | 177 | −54.945 | 34.880 | 66.868 | 1.00 | 53.97 | B |
| ATOM | 4662 | N | GLY | B | 178 | −52.995 | 33.758 | 66.845 | 1.00 | 55.05 | B |
| ATOM | 4663 | CA | GLY | B | 178 | −52.823 | 33.834 | 68.280 | 1.00 | 55.51 | B |
| ATOM | 4664 | C | GLY | B | 178 | −53.887 | 33.043 | 68.995 | 1.00 | 57.01 | B |
| ATOM | 4665 | O | GLY | B | 178 | −54.045 | 33.160 | 70.216 | 1.00 | 57.61 | B |
| ATOM | 4666 | N | GLY | B | 179 | −54.609 | 32.220 | 68.235 | 1.00 | 57.98 | B |
| ATOM | 4667 | CA | GLY | B | 179 | −55.680 | 31.417 | 68.807 | 1.00 | 57.98 | B |
| ATOM | 4668 | C | GLY | B | 179 | −55.244 | 30.118 | 69.448 | 1.00 | 57.76 | B |
| ATOM | 4669 | O | GLY | B | 179 | −54.053 | 29.825 | 69.536 | 1.00 | 56.43 | B |
| ATOM | 4670 | N | ASP | B | 180 | −56.223 | 29.341 | 69.901 | 1.00 | 59.50 | B |
| ATOM | 4671 | CA | ASP | B | 180 | −55.969 | 28.049 | 70.541 | 1.00 | 60.10 | B |
| ATOM | 4672 | CB | ASP | B | 180 | −56.438 | 28.094 | 71.996 | 1.00 | 63.11 | B |
| ATOM | 4673 | CG | ASP | B | 180 | −56.798 | 26.726 | 72.538 | 1.00 | 67.29 | B |
| ATOM | 4674 | OD1 | ASP | B | 180 | −57.987 | 26.343 | 72.423 | 1.00 | 70.24 | B |
| ATOM | 4675 | OD2 | ASP | B | 180 | −55.899 | 26.030 | 73.065 | 1.00 | 68.08 | B |
| ATOM | 4676 | C | ASP | B | 180 | −56.634 | 26.879 | 69.790 | 1.00 | 59.27 | B |
| ATOM | 4677 | O | ASP | B | 180 | −57.842 | 26.887 | 69.531 | 1.00 | 57.37 | B |
| ATOM | 4678 | N | PRO | B | 181 | −55.840 | 25.850 | 69.451 | 1.00 | 58.66 | B |
| ATOM | 4679 | CD | PRO | B | 181 | −54.475 | 25.708 | 69.983 | 1.00 | 58.94 | B |
| ATOM | 4680 | CA | PRO | B | 181 | −56.235 | 24.632 | 68.732 | 1.00 | 58.48 | B |
| ATOM | 4681 | CB | PRO | B | 181 | −55.073 | 23.685 | 68.980 | 1.00 | 57.50 | B |
| ATOM | 4682 | CG | PRO | B | 181 | −53.912 | 24.608 | 69.116 | 1.00 | 59.55 | B |
| ATOM | 4683 | C | PRO | B | 181 | −57.533 | 24.017 | 69.199 | 1.00 | 58.65 | B |
| ATOM | 4684 | O | PRO | B | 181 | −58.310 | 23.521 | 68.396 | 1.00 | 59.93 | B |
| ATOM | 4685 | N | ASP | B | 182 | −57.765 | 24.050 | 70.503 | 1.00 | 59.41 | B |
| ATOM | 4686 | CA | ASP | B | 182 | −58.965 | 23.458 | 71.075 | 1.00 | 60.53 | B |
| ATOM | 4687 | CB | ASP | B | 182 | −58.851 | 23.395 | 72.601 | 1.00 | 62.23 | B |
| ATOM | 4688 | CG | ASP | B | 182 | −57.608 | 22.638 | 73.075 | 1.00 | 64.81 | B |
| ATOM | 4689 | OD1 | ASP | B | 182 | −57.680 | 21.993 | 74.149 | 1.00 | 64.34 | B |
| ATOM | 4690 | OD2 | ASP | B | 182 | −56.557 | 22.697 | 72.391 | 1.00 | 66.45 | B |
| ATOM | 4691 | C | ASP | B | 182 | −60.275 | 24.138 | 70.707 | 1.00 | 60.99 | B |
| ATOM | 4692 | O | ASP | B | 182 | −61.331 | 23.522 | 70.801 | 1.00 | 60.91 | B |
| ATOM | 4693 | N | SER | B | 183 | −60.222 | 25.399 | 70.292 | 1.00 | 62.58 | B |
| ATOM | 4694 | CA | SER | B | 183 | −61.447 | 26.120 | 69.941 | 1.00 | 63.90 | B |
| ATOM | 4695 | CB | SER | B | 183 | −61.649 | 27.288 | 70.907 | 1.00 | 64.42 | B |
| ATOM | 4696 | OG | SER | B | 183 | −60.471 | 28.072 | 71.001 | 1.00 | 69.04 | B |
| ATOM | 4697 | C | SER | B | 183 | −61.471 | 26.623 | 68.501 | 1.00 | 64.02 | B |
| ATOM | 4698 | O | SER | B | 183 | −61.871 | 27.755 | 68.228 | 1.00 | 63.92 | B |
| ATOM | 4699 | N | LEU | B | 184 | −61.040 | 25.774 | 67.579 | 1.00 | 64.21 | B |
| ATOM | 4700 | CA | LEU | B | 184 | −61.022 | 26.136 | 66.180 | 1.00 | 64.75 | B |
| ATOM | 4701 | CB | LEU | B | 184 | −59.591 | 26.404 | 65.714 | 1.00 | 64.22 | B |
| ATOM | 4702 | CG | LEU | B | 184 | −59.141 | 27.868 | 65.706 | 1.00 | 65.22 | B |
| ATOM | 4703 | CD1 | LEU | B | 184 | −59.220 | 28.447 | 67.108 | 1.00 | 67.05 | B |
| ATOM | 4704 | CD2 | LEU | B | 184 | −57.718 | 27.963 | 65.172 | 1.00 | 66.24 | B |
| ATOM | 4705 | C | LEU | B | 184 | −61.617 | 24.991 | 65.393 | 1.00 | 66.29 | B |
| ATOM | 4706 | O | LEU | B | 184 | −61.308 | 23.838 | 65.662 | 1.00 | 66.51 | B |
| ATOM | 4707 | N | SER | B | 185 | −62.487 | 25.314 | 64.440 | 1.00 | 67.84 | B |
| ATOM | 4708 | CA | SER | B | 185 | −63.115 | 24.305 | 63.599 | 1.00 | 69.75 | B |
| ATOM | 4709 | CB | SER | B | 185 | −64.187 | 24.924 | 62.698 | 1.00 | 70.57 | B |
| ATOM | 4710 | OG | SER | B | 185 | −64.565 | 24.026 | 61.660 | 1.00 | 69.10 | B |
| ATOM | 4711 | C | SER | B | 185 | −62.041 | 23.726 | 62.724 | 1.00 | 71.65 | B |
| ATOM | 4712 | O | SER | B | 185 | −61.376 | 24.468 | 62.002 | 1.00 | 73.46 | B |
| ATOM | 4713 | N | MSE | B | 186 | −61.863 | 22.411 | 62.787 | 1.00 | 73.10 | B |
| ATOM | 4714 | CA | MSE | B | 186 | −60.858 | 21.747 | 61.969 | 1.00 | 75.27 | B |
| ATOM | 4715 | CB | MSE | B | 186 | −60.886 | 20.240 | 62.242 | 1.00 | 75.91 | B |
| ATOM | 4716 | CG | MSE | B | 186 | −59.992 | 19.394 | 61.345 | 1.00 | 77.23 | B |
| ATOM | 4717 | SE | MSE | B | 186 | −58.100 | 19.736 | 61.525 | 1.00 | 82.55 | B |
| ATOM | 4718 | CE | MSE | B | 186 | −57.554 | 19.538 | 59.673 | 1.00 | 80.37 | B |
| ATOM | 4719 | C | MSE | B | 186 | −61.197 | 22.031 | 60.507 | 1.00 | 76.45 | B |
| ATOM | 4720 | O | MSE | B | 186 | −60.350 | 22.468 | 59.718 | 1.00 | 75.84 | B |
| ATOM | 4721 | N | HIS | B | 187 | −62.464 | 21.794 | 60.180 | 1.00 | 78.52 | B |
| ATOM | 4722 | CA | HIS | B | 187 | −63.008 | 21.989 | 58.844 | 1.00 | 80.12 | B |
| ATOM | 4723 | CB | HIS | B | 187 | −64.512 | 21.713 | 58.878 | 1.00 | 82.55 | B |
| ATOM | 4724 | CG | HIS | B | 187 | −65.201 | 21.939 | 57.568 | 1.00 | 86.18 | B |
| ATOM | 4725 | CD2 | HIS | B | 187 | −66.365 | 22.564 | 57.267 | 1.00 | 86.87 | B |
| ATOM | 4726 | ND1 | HIS | B | 187 | −64.705 | 21.457 | 56.374 | 1.00 | 87.60 | B |
| ATOM | 4727 | CE1 | HIS | B | 187 | −65.534 | 21.774 | 55.395 | 1.00 | 87.66 | B |
| ATOM | 4728 | NE2 | HIS | B | 187 | −66.549 | 22.445 | 55.910 | 1.00 | 87.80 | B |
| ATOM | 4729 | C | HIS | B | 187 | −62.756 | 23.379 | 58.259 | 1.00 | 80.67 | B |
| ATOM | 4730 | O | HIS | B | 187 | −62.263 | 23.504 | 57.131 | 1.00 | 80.42 | B |
| ATOM | 4731 | N | ALA | B | 188 | −63.103 | 24.411 | 59.033 | 1.00 | 81.13 | B |
| ATOM | 4732 | CA | ALA | B | 188 | −62.949 | 25.807 | 58.622 | 1.00 | 80.64 | B |
| ATOM | 4733 | CB | ALA | B | 188 | −63.838 | 26.695 | 59.482 | 1.00 | 79.82 | B |
| ATOM | 4734 | C | ALA | B | 188 | −61.511 | 26.320 | 58.662 | 1.00 | 80.79 | B |
| ATOM | 4735 | O | ALA | B | 188 | −61.121 | 27.129 | 57.827 | 1.00 | 81.80 | B |
| ATOM | 4736 | N | SER | B | 189 | −60.721 | 25.857 | 59.625 | 1.00 | 80.68 | B |
| ATOM | 4737 | CA | SER | B | 189 | −59.336 | 26.299 | 59.729 | 1.00 | 81.08 | B |
| ATOM | 4738 | CB | SER | B | 189 | −58.776 | 25.982 | 61.116 | 1.00 | 81.86 | B |
| ATOM | 4739 | OG | SER | B | 189 | −59.452 | 26.721 | 62.122 | 1.00 | 81.94 | B |
| ATOM | 4740 | C | SER | B | 189 | −58.450 | 25.675 | 58.660 | 1.00 | 81.32 | B |
| ATOM | 4741 | O | SER | B | 189 | −57.738 | 26.385 | 57.958 | 1.00 | 82.34 | B |
| ATOM | 4742 | N | PHE | B | 190 | −58.478 | 24.353 | 58.536 | 1.00 | 81.32 | B |
| ATOM | 4743 | CA | PHE | B | 190 | −57.664 | 23.697 | 57.520 | 1.00 | 81.99 | B |
| ATOM | 4744 | CB | PHE | B | 190 | −56.712 | 22.668 | 58.139 | 1.00 | 81.69 | B |
| ATOM | 4745 | CG | PHE | B | 190 | −55.777 | 23.230 | 59.172 | 1.00 | 81.72 | B |
| ATOM | 4746 | CD1 | PHE | B | 190 | −56.239 | 23.564 | 60.444 | 1.00 | 81.23 | B |
| ATOM | 4747 | CD2 | PHE | B | 190 | −54.421 | 23.369 | 58.894 | 1.00 | 81.61 | B |
| ATOM | 4748 | CE1 | PHE | B | 190 | −55.361 | 24.022 | 61.427 | 1.00 | 80.70 | B |
| ATOM | 4749 | CE2 | PHE | B | 190 | −53.533 | 23.827 | 59.870 | 1.00 | 82.19 | B |
| ATOM | 4750 | CZ | PHE | B | 190 | −54.005 | 24.152 | 61.140 | 1.00 | 81.54 | B |
| ATOM | 4751 | C | PHE | B | 190 | −58.557 | 22.989 | 56.511 | 1.00 | 82.57 | B |
| ATOM | 4752 | O | PHE | B | 190 | −58.837 | 21.802 | 56.646 | 1.00 | 83.40 | B |
| ATOM | 4753 | N | PRO | B | 191 | −59.026 | 23.712 | 55.487 | 1.00 | 83.71 | B |
| ATOM | 4754 | CD | PRO | B | 191 | −58.926 | 25.169 | 55.303 | 1.00 | 84.25 | B |
| ATOM | 4755 | CA | PRO | B | 191 | −59.889 | 23.120 | 54.462 | 1.00 | 84.10 | B |
| ATOM | 4756 | CB | PRO | B | 191 | −60.307 | 24.324 | 53.624 | 1.00 | 84.31 | B |
| ATOM | 4757 | CG | PRO | B | 191 | −60.207 | 25.473 | 54.584 | 1.00 | 84.26 | B |
| ATOM | 4758 | C | PRO | B | 191 | −59.107 | 22.112 | 53.633 | 1.00 | 85.02 | B |
| ATOM | 4759 | O | PRO | B | 191 | −59.545 | 20.985 | 53.428 | 1.00 | 84.44 | B |
| ATOM | 4760 | N | ALA | B | 192 | −57.937 | 22.537 | 53.168 | 1.00 | 86.75 | B |
| ATOM | 4761 | CA | ALA | B | 192 | −57.074 | 21.699 | 52.346 | 1.00 | 88.71 | B |
| ATOM | 4762 | CB | ALA | B | 192 | −55.786 | 22.455 | 51.996 | 1.00 | 89.46 | B |
| ATOM | 4763 | C | ALA | B | 192 | −56.731 | 20.361 | 52.996 | 1.00 | 89.66 | B |
| ATOM | 4764 | O | ALA | B | 192 | −56.162 | 19.485 | 52.336 | 1.00 | 91.02 | B |
| ATOM | 4765 | N | LEU | B | 193 | −57.057 | 20.201 | 54.281 | 1.00 | 89.43 | B |
| ATOM | 4766 | CA | LEU | B | 193 | −56.784 | 18.941 | 54.979 | 1.00 | 88.91 | B |
| ATOM | 4767 | CB | LEU | B | 193 | −55.923 | 19.164 | 56.225 | 1.00 | 87.00 | B |
| ATOM | 4768 | CG | LEU | B | 193 | −54.452 | 19.521 | 56.018 | 1.00 | 85.59 | B |
| ATOM | 4769 | CD1 | LEU | B | 193 | −53.765 | 19.402 | 57.349 | 1.00 | 84.83 | B |
| ATOM | 4770 | CD2 | LEU | B | 193 | −53.789 | 18.590 | 55.017 | 1.00 | 83.72 | B |
| ATOM | 4771 | C | LEU | B | 193 | −58.052 | 18.199 | 55.376 | 1.00 | 89.34 | B |
| ATOM | 4772 | O | LEU | B | 193 | −58.062 | 16.972 | 55.427 | 1.00 | 88.93 | B |
| ATOM | 4773 | N | TRP | B | 194 | −59.116 | 18.936 | 55.670 | 1.00 | 90.39 | B |
| ATOM | 4774 | CA | TRP | B | 194 | −60.375 | 18.294 | 56.033 | 1.00 | 92.08 | B |
| ATOM | 4775 | CB | TRP | B | 194 | −61.399 | 19.344 | 56.474 | 1.00 | 92.09 | B |
| ATOM | 4776 | CG | TRP | B | 194 | −62.804 | 18.811 | 56.619 | 1.00 | 92.80 | B |
| ATOM | 4777 | CD2 | TRP | B | 194 | −63.413 | 18.318 | 57.818 | 1.00 | 92.81 | B |
| ATOM | 4778 | CE2 | TRP | B | 194 | −64.740 | 17.942 | 57.493 | 1.00 | 92.71 | B |
| ATOM | 4779 | CE3 | TRP | B | 194 | −62.968 | 18.157 | 59.138 | 1.00 | 93.21 | B |
| ATOM | 4780 | CD1 | TRP | B | 194 | −63.759 | 18.712 | 55.637 | 1.00 | 93.15 | B |
| ATOM | 4781 | NE1 | TRP | B | 194 | −64.925 | 18.193 | 56.158 | 1.00 | 92.27 | B |
| ATOM | 4782 | CZ2 | TRP | B | 194 | −65.627 | 17.415 | 58.444 | 1.00 | 92.57 | B |
| ATOM | 4783 | CZ3 | TRP | B | 194 | −63.854 | 17.630 | 60.088 | 1.00 | 93.07 | B |
| ATOM | 4784 | CH2 | TRP | B | 194 | −65.166 | 17.267 | 59.731 | 1.00 | 92.59 | B |
| ATOM | 4785 | C | TRP | B | 194 | −60.909 | 17.502 | 54.829 | 1.00 | 92.85 | B |
| ATOM | 4786 | O | TRP | B | 194 | −61.596 | 16.485 | 54.989 | 1.00 | 92.54 | B |
| ATOM | 4787 | N | ASN | B | 195 | −60.583 | 17.977 | 53.627 | 1.00 | 93.15 | B |
| ATOM | 4788 | CA | ASN | B | 195 | −61.013 | 17.322 | 52.397 | 1.00 | 93.31 | B |
| ATOM | 4789 | CB | ASN | B | 195 | −60.707 | 18.206 | 51.185 | .00 | 92.92 | B |
| ATOM | 4790 | CG | ASN | B | 195 | −61.639 | 19.397 | 51.082 | .00 | 92.64 | B |
| ATOM | 4791 | OD1 | ASN | B | 195 | −62.848 | 19.239 | 50.907 | .00 | 92.49 | B |
| ATOM | 4792 | ND2 | ASN | B | 195 | −61.083 | 20.598 | 51.192 | .00 | 92.47 | B |
| ATOM | 4793 | C | ASN | B | 195 | −60.300 | 15.986 | 52.257 | 1.00 | 93.87 | B |
| ATOM | 4794 | O | ASN | B | 195 | −60.944 | 14.938 | 52.192 | 1.00 | 93.69 | B |
| ATOM | 4795 | N | LEU | B | 196 | −58.970 | 16.028 | 52.224 | 1.00 | 94.28 | B |
| ATOM | 4796 | CA | LEU | B | 196 | −58.162 | 14.817 | 52.094 | 1.00 | 94.39 | B |
| ATOM | 4797 | CB | LEU | B | 196 | −56.678 | 15.142 | 52.297 | 1.00 | 94.34 | B |
| ATOM | 4798 | CG | LEU | B | 196 | −55.917 | 15.467 | 51.006 | 1.00 | 95.46 | B |
| ATOM | 4799 | CD1 | LEU | B | 196 | −56.649 | 16.555 | 50.233 | 1.00 | 95.00 | B |
| ATOM | 4800 | CD2 | LEU | B | 196 | −54.492 | 15.895 | 51.334 | 1.00 | 95.34 | B |
| ATOM | 4801 | C | LEU | B | 196 | −58.573 | 13.678 | 53.028 | 1.00 | 94.12 | B |
| ATOM | 4802 | O | LEU | B | 196 | −58.550 | 12.516 | 52.629 | 1.00 | 94.30 | B |
| ATOM | 4803 | N | GLU | B | 197 | −58.944 | 13.993 | 54.265 | 1.00 | 94.05 | B |
| ATOM | 4804 | CA | GLU | B | 197 | −59.354 | 12.938 | 55.178 | 1.00 | 94.26 | B |
| ATOM | 4805 | CB | GLU | B | 197 | −59.566 | 13.455 | 56.597 | 1.00 | 95.07 | B |
| ATOM | 4806 | CG | GLU | B | 197 | −60.117 | 12.370 | 57.524 | 1.00 | 95.69 | B |
| ATOM | 4807 | CD | GLU | B | 197 | −60.627 | 12.911 | 58.842 | 1.00 | 97.07 | B |
| ATOM | 4808 | OE1 | GLU | B | 197 | −61.510 | 13.799 | 58.825 | 1.00 | 97.55 | B |
| ATOM | 4809 | OE2 | GLU | B | 197 | −60.150 | 12.444 | 59.898 | 1.00 | 97.50 | B |
| ATOM | 4810 | C | GLU | B | 197 | −60.657 | 12.339 | 54.690 | 1.00 | 94.84 | B |
| ATOM | 4811 | O | GLU | B | 197 | −60.837 | 11.121 | 54.730 | 1.00 | 95.53 | B |
| ATOM | 4812 | N | LYS | B | 198 | −61.571 | 13.195 | 54.238 | 1.00 | 94.65 | B |
| ATOM | 4813 | CA | LYS | B | 198 | −62.866 | 12.733 | 53.735 | 1.00 | 94.49 | B |
| ATOM | 4814 | CB | LYS | B | 198 | −63.697 | 13.930 | 53.237 | 1.00 | 95.97 | B |
| ATOM | 4815 | CG | LYS | B | 198 | −65.045 | 13.566 | 52.597 | 1.00 | 96.88 | B |
| ATOM | 4816 | CD | LYS | B | 198 | −64.872 | 13.023 | 51.177 | 1.00 | 96.71 | B |
| ATOM | 4817 | CE | LYS | B | 198 | −66.193 | 12.588 | 50.566 | 1.00 | 96.51 | B |
| ATOM | 4818 | NZ | LYS | B | 198 | −66.001 | 12.124 | 49.162 | 1.00 | 96.21 | B |
| ATOM | 4819 | C | LYS | B | 198 | −62.685 | 11.704 | 52.610 | 1.00 | 92.98 | B |
| ATOM | 4820 | O | LYS | B | 198 | −63.313 | 10.643 | 52.621 | 1.00 | 91.84 | B |
| ATOM | 4821 | N | ALA | B | 199 | −61.823 | 12.034 | 51.648 | 1.00 | 91.77 | B |
| ATOM | 4822 | CA | ALA | B | 199 | −61.537 | 11.161 | 50.509 | 1.00 | 90.52 | B |
| ATOM | 4823 | CB | ALA | B | 199 | −60.996 | 11.990 | 49.331 | 1.00 | 89.39 | B |
| ATOM | 4824 | C | ALA | B | 199 | −60.534 | 10.064 | 50.875 | 1.00 | 89.69 | B |
| ATOM | 4825 | O | ALA | B | 199 | −60.919 | 8.934 | 51.189 | 1.00 | 89.78 | B |
| ATOM | 4826 | N | PHE | B | 200 | −59.251 | 10.410 | 50.837 | 1.00 | 88.11 | B |
| ATOM | 4827 | CA | PHE | B | 200 | −58.184 | 9.467 | 51.143 | 1.00 | 86.33 | B |
| ATOM | 4828 | CB | PHE | B | 200 | −56.832 | 10.165 | 50.985 | 1.00 | 87.33 | B |
| ATOM | 4829 | CG | PHE | B | 200 | −56.535 | 10.604 | 49.571 | 1.00 | 87.92 | B |
| ATOM | 4830 | CD1 | PHE | B | 200 | −56.347 | 9.664 | 48.562 | 1.00 | 87.56 | B |
| ATOM | 4831 | CD2 | PHE | B | 200 | −56.422 | 11.959 | 49.253 | 1.00 | 88.30 | B |
| ATOM | 4832 | CE1 | PHE | B | 200 | −56.048 | 10.066 | 47.254 | 1.00 | 87.68 | B |
| ATOM | 4833 | CE2 | PHE | B | 200 | −56.123 | 12.371 | 47.945 | 1.00 | 87.90 | B |
| ATOM | 4834 | CZ | PHE | B | 200 | −55.935 | 11.423 | 46.947 | 1.00 | 87.80 | B |
| ATOM | 4835 | C | PHE | B | 200 | −58.282 | 8.790 | 52.520 | 1.00 | 84.94 | B |
| ATOM | 4836 | O | PHE | B | 200 | −58.006 | 7.598 | 52.636 | 1.00 | 85.12 | B |
| ATOM | 4837 | N | GLY | B | 201 | −58.663 | 9.529 | 53.559 | 1.00 | 82.81 | B |
| ATOM | 4838 | CA | GLY | B | 201 | −58.782 | 8.919 | 54.879 | 1.00 | 80.05 | B |
| ATOM | 4839 | C | GLY | B | 201 | −57.581 | 9.028 | 55.824 | 1.00 | 78.47 | B |
| ATOM | 4840 | O | GLY | B | 201 | −57.719 | 8.926 | 57.053 | 1.00 | 78.16 | B |
| ATOM | 4841 | N | SER | B | 202 | −56.392 | 9.217 | 55.272 | 1.00 | 75.31 | B |
| ATOM | 4842 | CA | SER | B | 202 | −55.225 | 9.326 | 56.118 | 1.00 | 73.54 | B |
| ATOM | 4843 | CB | SER | B | 202 | −54.400 | 8.030 | 56.073 | 1.00 | 73.40 | B |
| ATOM | 4844 | OG | SER | B | 202 | −53.694 | 7.892 | 54.852 | 1.00 | 74.84 | B |
| ATOM | 4845 | C | SER | B | 202 | −54.403 | 10.509 | 55.645 | 1.00 | 72.11 | B |
| ATOM | 4846 | O | SER | B | 202 | −53.767 | 10.458 | 54.593 | 1.00 | 73.46 | B |
| ATOM | 4847 | N | VAL | B | 203 | −54.430 | 11.578 | 56.428 | 1.00 | 68.83 | B |
| ATOM | 4848 | CA | VAL | B | 203 | −53.705 | 12.789 | 56.095 | 1.00 | 66.26 | B |
| ATOM | 4849 | CB | VAL | B | 203 | −53.422 | 13.609 | 57.373 | 1.00 | 66.52 | B |
| ATOM | 4850 | CG1 | VAL | B | 203 | −52.681 | 14.892 | 57.030 | 1.00 | 66.10 | B |
| ATOM | 4851 | CG2 | VAL | B | 203 | −54.726 | 13.927 | 58.074 | 1.00 | 66.57 | B |
| ATOM | 4852 | C | VAL | B | 203 | −52.389 | 12.515 | 55.368 | 1.00 | 65.13 | B |
| ATOM | 4853 | O | VAL | B | 203 | −52.248 | 12.821 | 54.182 | 1.00 | 63.53 | B |
| ATOM | 4854 | N | ILE | B | 204 | −51.438 | 11.922 | 56.085 | 1.00 | 64.14 | B |
| ATOM | 4855 | CA | ILE | B | 204 | −50.118 | 11.645 | 55.536 | 1.00 | 62.82 | B |
| ATOM | 4856 | CB | ILE | B | 204 | −49.355 | 10.632 | 56.412 | 1.00 | 61.72 | B |
| ATOM | 4857 | CG2 | ILE | B | 204 | −48.282 | 9.921 | 55.592 | 1.00 | 61.76 | B |
| ATOM | 4858 | CG1 | ILE | B | 204 | −48.734 | 11.358 | 57.607 | 1.00 | 60.26 | B |
| ATOM | 4859 | CD1 | ILE | B | 204 | −47.659 | 12.344 | 57.233 | 1.00 | 58.31 | B |
| ATOM | 4860 | C | ILE | B | 204 | −50.136 | 11.146 | 54.108 | 1.00 | 62.61 | B |
| ATOM | 4861 | O | ILE | B | 204 | −49.407 | 11.642 | 53.256 | 1.00 | 62.13 | B |
| ATOM | 4862 | N | LEU | B | 205 | −50.976 | 10.162 | 53.849 | 1.00 | 63.35 | B |
| ATOM | 4863 | CA | LEU | B | 205 | −51.054 | 9.589 | 52.523 | 1.00 | 63.83 | B |
| ATOM | 4864 | CB | LEU | B | 205 | −51.962 | 8.374 | 52.552 | 1.00 | 60.04 | B |
| ATOM | 4865 | CG | LEU | B | 205 | −51.462 | 7.231 | 51.698 | 1.00 | 57.62 | B |
| ATOM | 4866 | CD1 | LEU | B | 205 | −50.058 | 6.865 | 52.116 | 1.00 | 56.79 | B |
| ATOM | 4867 | CD2 | LEU | B | 205 | −52.402 | 6.061 | 51.853 | 1.00 | 58.11 | B |
| ATOM | 4868 | C | LEU | B | 205 | −51.584 | 10.621 | 51.548 | 1.00 | 67.13 | B |
| ATOM | 4869 | O | LEU | B | 205 | −51.032 | 10.802 | 50.464 | 1.00 | 68.36 | B |
| ATOM | 4870 | N | GLY | B | 206 | −52.657 | 11.299 | 51.944 | 1.00 | 70.29 | B |
| ATOM | 4871 | CA | GLY | B | 206 | −53.247 | 12.321 | 51.101 | 1.00 | 73.05 | B |
| ATOM | 4872 | C | GLY | B | 206 | −52.225 | 13.378 | 50.731 | 1.00 | 75.30 | B |
| ATOM | 4873 | O | GLY | B | 206 | −51.950 | 13.591 | 49.551 | 1.00 | 75.65 | B |
| ATOM | 4874 | N | ALA | B | 207 | −51.656 | 14.037 | 51.736 | 1.00 | 77.39 | B |
| ATOM | 4875 | CA | ALA | B | 207 | −50.651 | 15.071 | 51.501 | 1.00 | 79.63 | B |
| ATOM | 4876 | CB | ALA | B | 207 | −49.931 | 15.407 | 52.800 | 1.00 | 78.62 | B |
| ATOM | 4877 | C | ALA | B | 207 | −49.643 | 14.616 | 50.446 | 1.00 | 81.44 | B |
| ATOM | 4878 | O | ALA | B | 207 | −49.317 | 15.363 | 49.523 | 1.00 | 82.41 | B |
| ATOM | 4879 | N | ILE | B | 208 | −49.152 | 13.390 | 50.579 | 1.00 | 83.07 | B |
| ATOM | 4880 | CA | ILE | B | 208 | −48.196 | 12.866 | 49.620 | 1.00 | 85.21 | B |
| ATOM | 4881 | CB | ILE | B | 208 | −47.710 | 11.462 | 50.036 | 1.00 | 84.02 | B |
| ATOM | 4882 | CG2 | ILE | B | 208 | −47.056 | 10.755 | 48.868 | 1.00 | 83.52 | B |
| ATOM | 4883 | CG1 | ILE | B | 208 | −46.714 | 11.593 | 51.190 | 1.00 | 82.94 | B |
| ATOM | 4884 | CD1 | ILE | B | 208 | −45.483 | 12.416 | 50.846 | 1.00 | 81.51 | B |
| ATOM | 4885 | C | ILE | B | 208 | −48.803 | 12.836 | 48.220 | 1.00 | 87.91 | B |
| ATOM | 4886 | O | ILE | B | 208 | −48.096 | 13.038 | 47.235 | 1.00 | 89.03 | B |
| ATOM | 4887 | N | ARG | B | 209 | −50.110 | 12.603 | 48.124 | 1.00 | 90.77 | B |
| ATOM | 4688 | CA | ARG | B | 209 | −50.773 | 12.591 | 46.817 | 1.00 | 94.35 | B |
| ATOM | 4889 | CB | ARG | B | 209 | −51.907 | 11.549 | 46.776 | 1.00 | 96.29 | B |
| ATOM | 4890 | CG | ARG | B | 209 | −52.497 | 11.297 | 45.368 | 1.00 | 98.01 | B |
| ATOM | 4891 | CD | ARG | B | 209 | −51.391 | 10.900 | 44.373 | 1.00 | 99.99 | B |
| ATOM | 4892 | NE | ARG | B | 209 | −51.879 | 10.553 | 43.034 | 1.00 | 100.00 | B |
| ATOM | 4893 | CZ | ARG | B | 209 | −51.086 | 10.351 | 41.980 | 1.00 | 100.00 | B |
| ATOM | 4894 | NH1 | ARG | B | 209 | −49.770 | 10.467 | 42.112 | 1.00 | 100.00 | B |
| ATOM | 4895 | NH2 | ARG | B | 209 | −51.603 | 10.032 | 40.795 | 1.00 | 99.46 | B |
| ATOM | 4896 | C | ARG | B | 209 | −51.338 | 13.982 | 46.498 | 1.00 | 95.56 | B |
| ATOM | 4897 | O | ARG | B | 209 | −52.461 | 14.112 | 45.995 | 1.00 | 96.32 | B |
| ATOM | 4898 | N | SER | B | 210 | −50.554 | 15.015 | 46.803 | 1.00 | 95.85 | B |
| ATOM | 4899 | CA | SER | B | 210 | −50.953 | 16.398 | 46.552 | 1.00 | 96.42 | B |
| ATOM | 4900 | CB | SER | B | 210 | −52.210 | 16.757 | 47.359 | 1.00 | 96.58 | B |
| ATOM | 4901 | OG | SER | B | 210 | −52.033 | 16.520 | 48.745 | 1.00 | 96.93 | B |
| ATOM | 4902 | C | SER | B | 210 | −49.823 | 17.374 | 46.872 | 1.00 | 96.50 | B |
| ATOM | 4903 | O | SER | B | 210 | −50.026 | 18.586 | 46.906 | 1.00 | 96.00 | B |
| ATOM | 4904 | N | ALA | B | 211 | −48.636 | 16.829 | 47.119 | 1.00 | 96.97 | B |
| ATOM | 4905 | CA | ALA | B | 211 | −47.439 | 17.623 | 47.401 | 1.00 | 97.64 | B |
| ATOM | 4906 | CB | ALA | B | 211 | −46.874 | 17.277 | 48.775 | 1.00 | 97.19 | B |
| ATOM | 4907 | C | ALA | B | 211 | −46.469 | 17.199 | 46.303 | 1.00 | 98.30 | B |
| ATOM | 4908 | O | ALA | B | 211 | −45.432 | 17.823 | 46.064 | 1.00 | 97.83 | B |
| ATOM | 4909 | N | ALA | B | 212 | −46.851 | 16.109 | 45.645 | 1.00 | 99.21 | B |
| ATOM | 4910 | CA | ALA | B | 212 | −46.121 | 15.506 | 44.537 | 1.00 | 99.97 | B |
| ATOM | 4911 | CB | ALA | B | 212 | −45.082 | 14.497 | 45.057 | 1.00 | 99.91 | B |
| ATOM | 4912 | C | ALA | B | 212 | −47.217 | 14.794 | 43.740 | 1.00 | 100.00 | B |
| ATOM | 4913 | O | ALA | B | 212 | −47.088 | 13.626 | 43.362 | 1.00 | 99.74 | B |
| ATOM | 4914 | N | ALA | B | 213 | −48.308 | 15.525 | 43.519 | 1.00 | 100.00 | B |
| ATOM | 4915 | CA | ALA | B | 213 | −49.474 | 15.032 | 42.793 | 1.00 | 100.00 | B |
| ATOM | 4916 | CB | ALA | B | 213 | −50.317 | 16.218 | 42.302 | 1.00 | 99.47 | B |
| ATOM | 4917 | C | ALA | B | 213 | −49.096 | 14.131 | 41.621 | 1.00 | 99.64 | B |
| ATOM | 4918 | O | ALA | B | 213 | −48.474 | 14.575 | 40.658 | 1.00 | 99.30 | B |
| ATOM | 4919 | N | ALA | B | 224 | −44.289 | 36.496 | 35.885 | .50 | 76.81 | B |
| ATOM | 4920 | CA | ALA | B | 224 | −43.327 | 35.511 | 35.402 | .50 | 77.37 | B |
| ATOM | 4921 | CB | ALA | B | 224 | −43.612 | 35.179 | 33.936 | .50 | 77.48 | B |
| ATOM | 4922 | C | ALA | B | 224 | −41.889 | 36.011 | 35.564 | .50 | 77.34 | B |
| ATOM | 4923 | O | ALA | B | 224 | −41.396 | 36.807 | 34.760 | .50 | 76.65 | B |
| ATOM | 4924 | N | ALA | B | 225 | −41.222 | 35.533 | 36.612 | .50 | 77.52 | B |
| ATOM | 4925 | CA | ALA | B | 225 | −39.846 | 35.924 | 36.902 | .50 | 77.13 | B |
| ATOM | 4926 | CB | ALA | B | 225 | −39.760 | 36.508 | 38.313 | .50 | 76.69 | B |
| ATOM | 4927 | C | ALA | B | 225 | −38.882 | 34.748 | 36.765 | .50 | 76.72 | B |
| ATOM | 4928 | O | ALA | B | 225 | −39.271 | 33.656 | 36.351 | .50 | 76.06 | B |
| ATOM | 4929 | N | ALA | B | 226 | −37.619 | 34.991 | 37.105 | .50 | 76.75 | B |
| ATOM | 4930 | CA | ALA | B | 226 | −36.582 | 33.968 | 37.040 | .50 | 76.99 | B |
| ATOM | 4931 | CB | ALA | B | 226 | −35.247 | 34.593 | 36.644 | .50 | 76.68 | B |
| ATOM | 4932 | C | ALA | B | 226 | −36.478 | 33.339 | 38.421 | .50 | 77.04 | B |
| ATOM | 4933 | O | ALA | B | 226 | −36.143 | 32.162 | 38.564 | .50 | 77.24 | B |
| ATOM | 4934 | N | ALA | B | 227 | −36.769 | 34.150 | 39.432 | .50 | 77.03 | B |
| ATOM | 4935 | CA | ALA | B | 227 | −36.749 | 33.721 | 40.825 | .50 | 76.99 | B |
| ATOM | 4936 | CB | ALA | B | 227 | −35.825 | 34.630 | 41.641 | .50 | 76.92 | B |
| ATOM | 4937 | C | ALA | B | 227 | −38.193 | 33.840 | 41.316 | .50 | 76.93 | B |
| ATOM | 4938 | O | ALA | B | 227 | −38.460 | 34.282 | 42.441 | .50 | 76.93 | B |
| ATOM | 4939 | N | ALA | B | 228 | −39.117 | 33.442 | 40.446 | .50 | 76.22 | B |
| ATOM | 4940 | CA | ALA | B | 228 | −40.544 | 33.511 | 40.729 | .50 | 75.71 | B |
| ATOM | 4941 | CB | ALA | B | 228 | −41.328 | 33.055 | 39.507 | .50 | 75.27 | B |
| ATOM | 4942 | C | ALA | B | 228 | −40.987 | 32.723 | 41.958 | .50 | 75.23 | B |
| ATOM | 4943 | O | ALA | B | 228 | −41.430 | 33.309 | 42.942 | .50 | 74.73 | B |
| ATOM | 4944 | N | ALA | B | 229 | −40.868 | 31.400 | 41.892 | .50 | 75.29 | B |
| ATOM | 4945 | CA | ALA | B | 229 | −41.271 | 30.518 | 42.988 | .50 | 75.04 | B |
| ATOM | 4946 | CB | ALA | B | 229 | −40.847 | 29.080 | 42.683 | .50 | 74.91 | B |
| ATOM | 4947 | C | ALA | B | 229 | −40.726 | 30.941 | 44.349 | .50 | 74.96 | B |
| ATOM | 4948 | O | ALA | B | 229 | −41.493 | 31.256 | 45.260 | .50 | 73.77 | B |
| ATOM | 4949 | N | ALA | B | 230 | −39.404 | 30.939 | 44.487 | .50 | 75.47 | B |
| ATOM | 4950 | CA | ALA | B | 230 | −38.767 | 31.319 | 45.744 | .50 | 77.25 | B |
| ATOM | 4951 | CB | ALA | B | 230 | −37.277 | 31.543 | 45.524 | .50 | 76.69 | B |
| ATOM | 4952 | C | ALA | B | 230 | −39.408 | 32.580 | 46.322 | .50 | 78.69 | B |
| ATOM | 4953 | O | ALA | B | 230 | −39.404 | 32.796 | 47.536 | .50 | 78.48 | B |
| ATOM | 4954 | N | ALA | B | 231 | −39.956 | 33.408 | 45.438 | .50 | 80.45 | B |
| ATOM | 4955 | CA | ALA | B | 231 | −40.609 | 34.647 | 45.835 | .50 | 81.68 | B |
| ATOM | 4956 | CB | ALA | B | 231 | −40.330 | 35.731 | 44.803 | .50 | 81.63 | B |
| ATOM | 4957 | C | ALA | B | 231 | −42.115 | 34.424 | 45.978 | .50 | 82.80 | B |
| ATOM | 4958 | O | ALA | B | 231 | −42.756 | 35.000 | 46.865 | .50 | 82.72 | B |
| ATOM | 4959 | N | ALA | B | 232 | −42.674 | 33.594 | 45.096 | 1.00 | 84.14 | B |
| ATOM | 4960 | CA | ALA | B | 232 | −44.103 | 33.282 | 45.140 | 1.00 | 84.72 | B |
| ATOM | 4961 | CB | ALA | B | 232 | −44.446 | 32.212 | 44.103 | 1.00 | 84.17 | B |
| ATOM | 4962 | C | ALA | B | 232 | −44.336 | 32.759 | 46.554 | 1.00 | 84.72 | B |
| ATOM | 4963 | O | ALA | B | 232 | −45.338 | 33.079 | 47.205 | 1.00 | 84.51 | B |
| ATOM | 4964 | N | ALA | B | 233 | −43.371 | 31.963 | 47.013 | 1.00 | 84.18 | B |
| ATOM | 4965 | CA | ALA | B | 233 | −43.376 | 31.393 | 48.358 | 1.00 | 82.43 | B |
| ATOM | 4966 | CB | ALA | B | 233 | −42.970 | 29.909 | 48.312 | 1.00 | 82.52 | B |
| ATOM | 4967 | C | ALA | B | 233 | −42.368 | 32.208 | 49.188 | 1.00 | 80.21 | B |
| ATOM | 4968 | O | ALA | B | 233 | −41.623 | 33.034 | 48.649 | 1.00 | 79.87 | B |
| ATOM | 4969 | N | GLY | B | 234 | −42.333 | 31.967 | 50.491 | 1.00 | 77.33 | B |
| ATOM | 4970 | CA | GLY | B | 234 | −41.434 | 32.729 | 51.334 | 1.00 | 74.51 | B |
| ATOM | 4971 | C | GLY | B | 234 | −42.129 | 34.053 | 51.570 | 1.00 | 72.44 | B |
| ATOM | 4972 | O | GLY | B | 234 | −42.427 | 34.775 | 50.615 | 1.00 | 72.98 | B |
| ATOM | 4973 | N | SER | B | 235 | −42.415 | 34.367 | 52.829 | 1.00 | 69.31 | B |
| ATOM | 4974 | CA | SER | B | 235 | −43.100 | 35.608 | 53.156 | 1.00 | 65.19 | B |
| ATOM | 4975 | CB | SER | B | 235 | −43.623 | 35.539 | 54.590 | 1.00 | 64.55 | B |
| ATOM | 4976 | OG | SER | B | 235 | −42.605 | 35.117 | 55.477 | 1.00 | 63.74 | B |
| ATOM | 4977 | C | SER | B | 235 | −42.178 | 36.810 | 52.970 | 1.00 | 63.25 | B |
| ATOM | 4978 | O | SER | B | 235 | −40.955 | 36.669 | 53.005 | 1.00 | 63.82 | B |
| ATOM | 4979 | N | PHE | B | 236 | −42.769 | 37.985 | 52.761 | 1.00 | 60.15 | B |
| ATOM | 4980 | CA | PHE | B | 236 | −42.007 | 39.219 | 52.556 | 1.00 | 57.87 | B |
| ATOM | 4981 | CB | PHE | B | 236 | −41.665 | 39.374 | 51.068 | 1.00 | 57.40 | B |
| ATOM | 4982 | CG | PHE | B | 236 | −42.875 | 39.392 | 50.178 | 1.00 | 57.87 | B |
| ATOM | 4983 | CD1 | PHE | B | 236 | −43.717 | 40.496 | 50.148 | 1.00 | 57.16 | B |
| ATOM | 4984 | CD2 | PHE | B | 236 | −43.237 | 38.261 | 49.455 | 1.00 | 57.30 | B |
| ATOM | 4985 | CE1 | PHE | B | 236 | −44.906 | 40.468 | 49.424 | 1.00 | 58.37 | B |
| ATOM | 4986 | CE2 | PHE | B | 236 | −44.420 | 38.226 | 48.731 | 1.00 | 57.37 | B |
| ATOM | 4987 | CZ | PHE | B | 236 | −45.257 | 39.329 | 48.717 | 1.00 | 57.81 | B |
| ATOM | 4988 | C | PHE | B | 236 | −42.786 | 40.454 | 53.033 | 1.00 | 56.27 | B |
| ATOM | 4989 | O | PHE | B | 236 | −43.857 | 40.344 | 53.626 | 1.00 | 52.96 | B |
| ATOM | 4990 | N | SER | B | 237 | −42.228 | 41.628 | 52.766 | 1.00 | 55.99 | B |
| ATOM | 4991 | CA | SER | B | 237 | −42.862 | 42.885 | 53.141 | 1.00 | 57.08 | B |
| ATOM | 4992 | CB | SER | B | 237 | −42.437 | 43.291 | 54.556 | 1.00 | 56.53 | B |
| ATOM | 4993 | OG | SER | B | 237 | −43.162 | 44.431 | 54.998 | 1.00 | 55.19 | B |
| ATOM | 4994 | C | SER | B | 237 | −42.462 | 43.964 | 52.125 | 1.00 | 57.67 | B |
| ATOM | 4995 | O | SER | B | 237 | −42.167 | 43.649 | 50.967 | 1.00 | 58.91 | B |
| ATOM | 4996 | N | PHE | B | 238 | −42.464 | 45.226 | 52.543 | 1.00 | 57.26 | B |
| ATOM | 4997 | CA | PHE | B | 238 | −42.088 | 46.325 | 51.654 | 1.00 | 56.86 | B |
| ATOM | 4998 | CB | PHE | B | 238 | −43.320 | 46.979 | 51.034 | 1.00 | 56.46 | B |
| ATOM | 4999 | CG | PHE | B | 238 | −44.209 | 46.024 | 50.300 | 1.00 | 55.96 | B |
| ATOM | 5000 | CD1 | PHE | B | 238 | −45.195 | 45.315 | 50.978 | 1.00 | 53.42 | B |
| ATOM | 5001 | CD2 | PHE | B | 238 | −44.061 | 45.831 | 48.927 | 1.00 | 55.08 | B |
| ATOM | 5002 | CE1 | PHE | B | 238 | −46.013 | 44.438 | 50.301 | 1.00 | 54.07 | B |
| ATOM | 5003 | CE2 | PHE | B | 238 | −44.883 | 44.944 | 48.238 | 1.00 | 54.43 | B |
| ATOM | 5004 | CZ | PHE | B | 238 | −45.860 | 44.246 | 48.921 | 1.00 | 54.05 | B |
| ATOM | 5005 | C | PHE | B | 238 | −41.320 | 47.382 | 52.419 | 1.00 | 57.48 | B |
| ATOM | 5006 | O | PHE | B | 238 | −41.647 | 47.680 | 53.562 | 1.00 | 57.86 | B |
| ATOM | 5007 | N | ALA | B | 239 | −40.300 | 47.952 | 51.787 | 1.00 | 58.36 | B |
| ATOM | 5008 | CA | ALA | B | 239 | −39.496 | 48.976 | 52.436 | 1.00 | 58.66 | B |
| ATOM | 5009 | CB | ALA | B | 239 | −38.601 | 49.690 | 51.401 | 1.00 | 59.25 | B |
| ATOM | 5010 | C | ALA | B | 239 | −40.441 | 49.971 | 53.102 | 1.00 | 58.48 | B |
| ATOM | 5011 | O | ALA | B | 239 | −41.242 | 50.622 | 52.426 | 1.00 | 57.90 | B |
| ATOM | 5012 | N | GLY | B | 240 | −40.357 | 50.069 | 54.427 | 1.00 | 58.43 | B |
| ATOM | 5013 | CA | GLY | B | 240 | −41.207 | 50.996 | 55.151 | 1.00 | 58.60 | B |
| ATOM | 5014 | C | GLY | B | 240 | −42.335 | 50.343 | 55.929 | 1.00 | 59.69 | B |
| ATOM | 5015 | O | GLY | B | 240 | −42.992 | 50.992 | 56.732 | 1.00 | 59.04 | B |
| ATOM | 5016 | N | GLY | B | 241 | −42.566 | 49.055 | 55.702 | 1.00 | 61.89 | B |
| ATOM | 5017 | CA | GLY | B | 241 | −43.631 | 48.362 | 56.415 | 1.00 | 62.79 | B |
| ATOM | 5018 | C | GLY | B | 241 | −44.745 | 47.847 | 55.518 | 1.00 | 63.15 | B |
| ATOM | 5019 | O | GLY | B | 241 | −44.803 | 48.159 | 54.322 | 1.00 | 63.17 | B |
| ATOM | 5020 | N | MSE | B | 242 | −45.647 | 47.061 | 56.094 | 1.00 | 62.45 | B |
| ATOM | 5021 | CA | MSE | B | 242 | −46.744 | 46.499 | 55.314 | 1.00 | 62.33 | B |
| ATOM | 5022 | CB | MSE | B | 242 | −47.553 | 45.503 | 56.144 | 1.00 | 63.42 | B |
| ATOM | 5023 | CG | MSE | B | 242 | −46.896 | 44.146 | 56.293 | 1.00 | 65.28 | B |
| ATOM | 5024 | SE | MSE | B | 242 | −46.625 | 43.342 | 54.586 | 1.00 | 68.84 | B |
| ATOM | 5025 | CE | MSE | B | 242 | −48.446 | 42.818 | 54.248 | 1.00 | 68.02 | B |
| ATOM | 5026 | C | MSE | B | 242 | −47.682 | 47.547 | 54.771 | 1.00 | 62.12 | B |
| ATOM | 5027 | O | MSE | B | 242 | −48.362 | 47.310 | 53.779 | 1.00 | 62.43 | B |
| ATOM | 5028 | N | GLN | B | 243 | −47.713 | 48.712 | 55.410 | 1.00 | 61.87 | B |
| ATOM | 5029 | CA | GLN | B | 243 | −48.609 | 49.776 | 54.975 | 1.00 | 59.77 | B |
| ATOM | 5030 | CB | GLN | B | 243 | −48.614 | 50.920 | 55.998 | 1.00 | 60.26 | B |
| ATOM | 5031 | CG | GLN | B | 243 | −49.954 | 51.651 | 56.071 | 1.00 | 62.07 | B |
| ATOM | 5032 | CD | GLN | B | 243 | −49.954 | 52.868 | 56.998 | 1.00 | 63.09 | B |
| ATOM | 5033 | OE1 | GLN | B | 243 | −49.675 | 52.761 | 58.192 | 1.00 | 62.45 | B |
| ATOM | 5034 | NE2 | GLN | B | 243 | −50.282 | 54.032 | 56.443 | 1.00 | 64.02 | B |
| ATOM | 5035 | C | GLN | B | 243 | −48.287 | 50.312 | 53.579 | 1.00 | 57.96 | B |
| ATOM | 5036 | O | GLN | B | 243 | −49.169 | 50.820 | 52.904 | 1.00 | 56.88 | B |
| ATOM | 5037 | N | THR | B | 244 | −47.038 | 50.187 | 53.140 | 1.00 | 57.80 | 8 |
| ATOM | 5038 | CA | THR | B | 244 | −46.651 | 50.683 | 51.814 | 1.00 | 58.06 | B |
| ATOM | 5039 | CB | THR | B | 244 | −45.187 | 50.323 | 51.459 | 1.00 | 57.71 | B |
| ATOM | 5040 | OG1 | THR | B | 244 | −44.294 | 51.052 | 52.310 | 1.00 | 56.91 | B |
| ATOM | 5041 | CG2 | THR | B | 244 | −44.887 | 50.662 | 49.995 | 1.00 | 55.70 | B |
| ATOM | 5042 | C | THR | B | 244 | −47.548 | 50.162 | 50.693 | 1.00 | 59.09 | B |
| ATOM | 5043 | O | THR | B | 244 | −47.742 | 50.846 | 49.686 | 1.00 | 59.47 | B |
| ATOM | 5044 | N | LEU | B | 245 | −48.072 | 48.947 | 50.861 | 1.00 | 58.94 | B |
| ATOM | 5045 | CA | LEU | B | 245 | −48.961 | 48.346 | 49.871 | 1.00 | 58.22 | B |
| ATOM | 5046 | CB | LEU | B | 245 | −49.219 | 46.887 | 50.230 | 1.00 | 57.73 | B |
| ATOM | 5047 | CG | LEU | B | 245 | −50.385 | 46.154 | 49.571 | 1.00 | 57.63 | B |
| ATOM | 5048 | CD1 | LEU | B | 245 | −50.344 | 46.314 | 48.052 | 1.00 | 57.72 | B |
| ATOM | 5049 | CD2 | LEU | B | 245 | −50.305 | 44.682 | 49.969 | 1.00 | 57.77 | B |
| ATOM | 5050 | C | LEU | B | 245 | −50.266 | 49.126 | 49.873 | 1.00 | 58.51 | B |
| ATOM | 5051 | O | LEU | B | 245 | −50.745 | 49.582 | 48.851 | 1.00 | 59.67 | B |
| ATOM | 5052 | N | THR | B | 246 | −50.834 | 49.283 | 51.051 | 1.00 | 59.66 | B |
| ATOM | 5053 | CA | THR | B | 246 | −52.066 | 50.024 | 51.203 | 1.00 | 60.49 | B |
| ATOM | 5054 | CB | THR | B | 246 | −52.416 | 50.134 | 52.698 | 1.00 | 58.36 | B |
| ATOM | 5055 | OG1 | THR | B | 246 | −53.242 | 49.029 | 53.064 | 1.00 | 56.70 | B |
| ATOM | 5056 | CG2 | THR | B | 246 | −53.132 | 51.439 | 53.004 | 1.00 | 59.91 | B |
| ATOM | 5057 | C | THR | B | 246 | −51.963 | 51.424 | 50.589 | 1.00 | 62.74 | B |
| ATOM | 5058 | O | THR | B | 246 | −52.899 | 51.890 | 49.930 | 1.00 | 62.58 | B |
| ATOM | 5059 | N | ASP | B | 247 | −50.823 | 52.082 | 50.807 | 1.00 | 64.39 | B |
| ATOM | 5060 | CA | ASP | B | 247 | −50.605 | 53.442 | 50.314 | 1.00 | 65.92 | B |
| ATOM | 5061 | CB | ASP | B | 247 | −49.331 | 54.035 | 50.916 | 1.00 | 67.00 | B |
| ATOM | 5062 | CG | ASP | B | 247 | −49.411 | 54.205 | 52.427 | 1.00 | 69.18 | B |
| ATOM | 5063 | OD1 | ASP | B | 247 | −50.489 | 53.944 | 53.021 | 1.00 | 68.56 | B |
| ATOM | 5064 | OD2 | ASP | B | 247 | −48.381 | 54.610 | 53.019 | 1.00 | 70.04 | B |
| ATOM | 5065 | C | ASP | B | 247 | −50.523 | 53.525 | 48.800 | 1.00 | 66.62 | B |
| ATOM | 5066 | O | ASP | B | 247 | −50.958 | 54.509 | 48.204 | 1.00 | 67.55 | B |
| ATOM | 5067 | N | ALA | B | 248 | −49.958 | 52.502 | 48.174 | 1.00 | 66.03 | B |
| ATOM | 5068 | CA | ALA | B | 248 | −49.848 | 52.503 | 46.726 | 1.00 | 66.23 | B |
| ATOM | 5069 | CB | ALA | B | 248 | −49.115 | 51.270 | 46.256 | 1.00 | 65.07 | B |
| ATOM | 5070 | C | ALA | B | 248 | −51.243 | 52.552 | 46.116 | 1.00 | 67.41 | B |
| ATOM | 5071 | O | ALA | B | 248 | −51.462 | 53.232 | 45.113 | 1.00 | 68.02 | B |
| ATOM | 5072 | N | ILE | B | 249 | −52.187 | 51.835 | 46.723 | 1.00 | 67.62 | B |
| ATOM | 5073 | CA | ILE | B | 249 | −53.556 | 51.819 | 46.217 | 1.00 | 68.03 | B |
| ATOM | 5074 | CB | ILE | B | 249 | −54.417 | 50.753 | 46.908 | 1.00 | 68.35 | B |
| ATOM | 5075 | CG2 | ILE | B | 249 | −55.852 | 50.832 | 46.395 | 1.00 | 66.35 | B |
| ATOM | 5076 | CG1 | ILE | B | 249 | −53.826 | 49.365 | 46.669 | 1.00 | 69.24 | B |
| ATOM | 5077 | CD1 | ILE | B | 249 | −54.535 | 48.256 | 47.463 | 1.00 | 69.67 | B |
| ATOM | 5078 | C | ILE | B | 249 | −54.231 | 53.159 | 46.446 | 1.00 | 68.46 | B |
| ATOM | 5079 | O | ILE | B | 249 | −55.069 | 53.568 | 45.651 | 1.00 | 67.83 | B |
| ATOM | 5080 | N | CYS | B | 250 | −53.864 | 53.836 | 47.533 | 1.00 | 69.84 | B |
| ATOM | 5081 | CA | CYS | B | 250 | −54.460 | 55.130 | 47.877 | 1.00 | 72.18 | B |
| ATOM | 5082 | CB | CYS | B | 250 | −54.218 | 55.465 | 49.346 | 1.00 | 70.84 | B |
| ATOM | 5083 | SG | CYS | B | 250 | −55.377 | 54.668 | 50.431 | 1.00 | 73.74 | B |
| ATOM | 5084 | C | CYS | B | 250 | −54.002 | 56.302 | 47.033 | 1.00 | 73.74 | B |
| ATOM | 5085 | O | CYS | B | 250 | −54.669 | 57.338 | 46.998 | 1.00 | 74.75 | B |
| ATOM | 5086 | N | LYS | B | 251 | −52.862 | 56.151 | 46.368 | 1.00 | 75.36 | B |
| ATOM | 5087 | CA | LYS | B | 251 | −52.349 | 57.215 | 45.516 | 1.00 | 76.92 | B |
| ATOM | 5088 | CB | LYS | B | 251 | −50.829 | 57.104 | 45.363 | 1.00 | 75.51 | B |
| ATOM | 5089 | CG | LYS | B | 251 | −50.036 | 57.262 | 46.659 | 1.00 | 74.75 | B |
| ATOM | 5090 | CD | LYS | B | 251 | −50.271 | 58.604 | 47.343 | .00 | 74.89 | B |
| ATOM | 5091 | CE | LYS | B | 251 | −51.536 | 58.599 | 48.190 | .00 | 74.80 | B |
| ATOM | 5092 | NZ | LYS | B | 251 | −51.756 | 59.904 | 48.870 | .00 | 74.83 | B |
| ATOM | 5093 | C | LYS | B | 251 | −53.025 | 57.110 | 44.152 | 1.00 | 78.93 | B |
| ATOM | 5094 | O | LYS | B | 251 | −52.696 | 57.849 | 43.219 | 1.00 | 80.37 | B |
| ATOM | 5095 | N | ASP | B | 252 | −53.971 | 56.182 | 44.046 | 1.00 | 80.13 | B |
| ATOM | 5096 | CA | ASP | B | 252 | −54.711 | 55.968 | 42.812 | 1.00 | 81.47 | B |
| ATOM | 5097 | CB | ASP | B | 252 | −54.357 | 54.609 | 42.212 | 1.00 | 83.23 | B |
| ATOM | 5098 | CG | ASP | B | 252 | −52.988 | 54.602 | 41.561 | 1.00 | 85.40 | B |
| ATOM | 5099 | OD1 | ASP | B | 252 | −52.045 | 55.170 | 42.157 | 1.00 | 86.98 | B |
| ATOM | 5100 | OD2 | ASP | B | 252 | −52.851 | 54.024 | 40.459 | 1.00 | 86.23 | B |
| ATOM | 5101 | C | ASP | B | 252 | −56.196 | 56.038 | 43.096 | 1.00 | 81.86 | B |
| ATOM | 5102 | O | ASP | B | 252 | −56.979 | 55.282 | 42.537 | 1.00 | 81.24 | B |
| ATOM | 5103 | N | LEU | B | 253 | −56.567 | 56.953 | 43.981 | 1.00 | 82.98 | B |
| ATOM | 5104 | CA | LEU | B | 253 | −57.957 | 57.156 | 44.361 | 1.00 | 85.66 | B |
| ATOM | 5105 | CB | LEU | B | 253 | −58.316 | 56.280 | 45.568 | 1.00 | 85.49 | B |
| ATOM | 5106 | CG | LEU | B | 253 | −58.215 | 54.754 | 45.450 | 1.00 | 85.58 | B |
| ATOM | 5107 | CD1 | LEU | B | 253 | −58.517 | 54.119 | 46.802 | 1.00 | 85.22 | B |
| ATOM | 5108 | CD2 | LEU | B | 253 | −59.182 | 54.244 | 44.396 | 1.00 | 85.13 | B |
| ATOM | 5109 | C | LEU | B | 253 | −58.128 | 58.629 | 44.727 | 1.00 | 87.90 | B |
| ATOM | 5110 | O | LEU | B | 253 | −57.811 | 59.036 | 45.847 | 1.00 | 89.07 | B |
| ATOM | 5111 | N | ARG | B | 254 | −58.625 | 59.427 | 43.787 | 1.00 | 89.52 | B |
| ATOM | 5112 | CA | ARG | B | 254 | −58.811 | 60.856 | 44.029 | 1.00 | 91.46 | B |
| ATOM | 5113 | CB | ARG | B | 254 | −59.587 | 61.495 | 42.874 | 1.00 | 93.48 | B |
| ATOM | 5114 | CG | ARG | B | 254 | −58.704 | 61.885 | 41.683 | 1.00 | 96.69 | B |
| ATOM | 5115 | CD | ARG | B | 254 | −57.706 | 63.002 | 42.050 | 1.00 | 97.98 | B |
| ATOM | 5116 | NE | ARG | B | 254 | −56.969 | 63.491 | 40.882 | 1.00 | 99.09 | B |
| ATOM | 5117 | CZ | ARG | B | 254 | −56.153 | 64.546 | 40.885 | 1.00 | 99.36 | B |
| ATOM | 5118 | NH1 | ARG | B | 254 | −55.956 | 65.241 | 41.996 | 1.00 | 99.17 | B |
| ATOM | 5119 | NH2 | ARG | B | 254 | −55.535 | 64.911 | 39.769 | 1.00 | 99.28 | B |
| ATOM | 5120 | C | ARG | B | 254 | −59.482 | 61.187 | 45.357 | 1.00 | 91.23 | B |
| ATOM | 5121 | O | ARG | B | 254 | −60.057 | 60.322 | 46.012 | 1.00 | 91.19 | B |
| ATOM | 5122 | N | GLU | B | 255 | −59.391 | 62.455 | 45.742 | 1.00 | 91.54 | B |
| ATOM | 5123 | CA | GLU | B | 255 | −59.951 | 62.948 | 46.997 | 1.00 | 91.83 | B |
| ATOM | 5124 | CB | GLU | B | 255 | −59.788 | 64.474 | 47.064 | 1.00 | 92.62 | B |
| ATOM | 5125 | CG | GLU | B | 255 | −58.339 | 64.955 | 46.965 | 1.00 | 92.72 | B |
| ATOM | 5126 | CD | GLU | B | 255 | −58.214 | 66.462 | 47.073 | .00 | 92.63 | B |
| ATOM | 5127 | OE1 | GLU | B | 255 | −58.635 | 67.022 | 48.107 | .00 | 92.60 | B |
| ATOM | 5128 | OE2 | GLU | B | 255 | −57.693 | 67.086 | 46.125 | .00 | 92.60 | B |
| ATOM | 5129 | C | GLU | B | 255 | −61.417 | 62.578 | 47.240 | 1.00 | 91.31 | B |
| ATOM | 5130 | O | GLU | B | 255 | −61.853 | 62.435 | 48.389 | 1.00 | 90.89 | B |
| ATOM | 5131 | N | ASP | B | 256 | −62.175 | 62.430 | 46.159 | 1.00 | 90.47 | B |
| ATOM | 5132 | CA | ASP | B | 256 | −63.588 | 62.088 | 46.263 | 1.00 | 89.88 | B |
| ATOM | 5133 | CB | ASP | B | 256 | −64.333 | 62.569 | 45.012 | 1.00 | 90.46 | B |
| ATOM | 5134 | CG | ASP | B | 256 | −63.706 | 62.062 | 43.727 | .00 | 90.33 | B |
| ATOM | 5135 | OD1 | ASP | B | 256 | −64.256 | 62.349 | 42.643 | .00 | 90.41 | B |
| ATOM | 5136 | OD2 | ASP | B | 256 | −62.662 | 61.380 | 43.798 | .00 | 90.41 | B |
| ATOM | 5137 | C | ASP | B | 256 | −63.777 | 60.584 | 46.430 | 1.00 | 88.81 | B |
| ATOM | 5138 | O | ASP | B | 256 | −64.901 | 60.096 | 46.564 | 1.00 | 90.28 | B |
| ATOM | 5139 | N | GLU | B | 257 | −62.667 | 59.858 | 46.432 | 1.00 | 86.42 | B |
| ATOM | 5140 | CA | GLU | B | 257 | −62.695 | 58.409 | 46.561 | 1.00 | 83.70 | B |
| ATOM | 5141 | CB | GLU | B | 257 | −61.557 | 57.793 | 45.759 | 1.00 | 85.28 | B |
| ATOM | 5142 | CG | GLU | B | 257 | −62.001 | 57.038 | 44.524 | 1.00 | 87.85 | B |
| ATOM | 5143 | CD | GLU | B | 257 | −62.377 | 57.956 | 43.378 | 1.00 | 89.58 | B |
| ATOM | 5144 | OE1 | GLU | B | 257 | −63.196 | 58.876 | 43.609 | 1.00 | 90.92 | B |
| ATOM | 5145 | OE2 | GLU | B | 257 | −61.860 | 57.754 | 42.251 | 1.00 | 89.09 | B |
| ATOM | 5146 | C | GLU | B | 257 | −62.598 | 57.893 | 47.987 | 1.00 | 81.91 | B |
| ATOM | 5147 | O | GLU | B | 257 | −63.398 | 57.053 | 48.395 | 1.00 | 81.75 | B |
| ATOM | 5148 | N | LEU | B | 258 | −61.612 | 58.383 | 48.736 | 1.00 | 78.78 | B |
| ATOM | 5149 | CA | LEU | B | 258 | −61.399 | 57.940 | 50.111 | 1.00 | 75.60 | B |
| ATOM | 5150 | CB | LEU | B | 258 | −59.896 | 57.735 | 50.350 | 1.00 | 75.24 | B |
| ATOM | 5151 | CG | LEU | B | 258 | −59.442 | 57.117 | 51.673 | .00 | 74.56 | B |
| ATOM | 5152 | CD1 | LEU | B | 258 | −60.030 | 55.723 | 51.813 | .00 | 74.29 | B |
| ATOM | 5153 | CD2 | LEU | B | 258 | −57.921 | 57.061 | 51.713 | .00 | 74.14 | B |
| ATOM | 5154 | C | LEU | B | 258 | −61.955 | 58.921 | 51.131 | 1.00 | 73.31 | B |
| ATOM | 5155 | O | LEU | B | 258 | −61.504 | 60.050 | 51.201 | 1.00 | 74.70 | B |
| ATOM | 5156 | N | ARG | B | 259 | −62.927 | 58.493 | 51.926 | 1.00 | 71.36 | B |
| ATOM | 5157 | CA | ARG | B | 259 | −63.511 | 59.365 | 52.945 | 1.00 | 70.95 | B |
| ATOM | 5158 | CB | ARG | B | 259 | −65.030 | 59.429 | 52.780 | 1.00 | 72.28 | B |
| ATOM | 5159 | CG | ARG | B | 259 | −65.505 | 59.745 | 51.372 | 1.00 | 73.12 | B |
| ATOM | 5160 | CD | ARG | B | 259 | −66.979 | 60.098 | 51.380 | .00 | 73.69 | B |
| ATOM | 5161 | NE | ARG | B | 259 | −67.208 | 61.411 | 51.975 | .00 | 74.41 | B |
| ATOM | 5162 | CZ | ARG | B | 259 | −68.405 | 61.890 | 52.295 | .00 | 74.79 | B |
| ATOM | 5163 | NH1 | ARG | B | 259 | −69.491 | 61.161 | 52.082 | .00 | 75.00 | B |
| ATOM | 5164 | NH2 | ARG | B | 259 | −68.518 | 63.103 | 52.819 | .00 | 75.06 | B |
| ATOM | 5165 | C | ARG | B | 259 | −63.184 | 58.853 | 54.352 | 1.00 | 70.37 | B |
| ATOM | 5166 | O | ARG | B | 259 | −63.849 | 57.954 | 54.867 | 1.00 | 71.62 | B |
| ATOM | 5167 | N | LEU | B | 260 | −62.181 | 59.444 | 54.987 | 1.00 | 68.00 | B |
| ATOM | 5168 | CA | LEU | B | 260 | −61.761 | 59.005 | 56.317 | 1.00 | 67.01 | B |
| ATOM | 5169 | CB | LEU | B | 260 | −60.296 | 59.415 | 56.524 | 1.00 | 66.25 | B |
| ATOM | 5170 | CG | LEU | B | 260 | −59.349 | 59.064 | 55.361 | 1.00 | 64.39 | B |
| ATOM | 5171 | CD1 | LEU | B | 260 | −58.057 | 59.866 | 55.480 | 1.00 | 63.81 | B |
| ATOM | 5172 | CD2 | LEU | B | 260 | −59.070 | 57.571 | 55.338 | 1.00 | 62.17 | B |
| ATOM | 5173 | C | LEU | B | 260 | −62.636 | 59.511 | 57.480 | 1.00 | 66.42 | B |
| ATOM | 5174 | O | LEU | B | 260 | −63.287 | 60.528 | 57.367 | 1.00 | 66.06 | B |
| ATOM | 5175 | N | ASN | B | 261 | −62.664 | 58.779 | 58.590 | .00 | 67.54 | B |
| ATOM | 5176 | CA | ASN | B | 261 | −63.469 | 59.194 | 59.743 | .00 | 68.50 | B |
| ATOM | 5177 | CB | ASN | B | 261 | −63.144 | 60.628 | 60.116 | .00 | 68.57 | B |
| ATOM | 5178 | CG | ASN | B | 261 | −61.694 | 60.826 | 60.356 | 1.00 | 68.57 | B |
| ATOM | 5179 | OD1 | ASN | B | 261 | −61.172 | 60.426 | 61.392 | 1.00 | 69.51 | B |
| ATOM | 5180 | ND2 | ASN | B | 261 | −61.013 | 61.437 | 59.396 | 1.00 | 68.90 | B |
| ATOM | 5181 | C | ASN | B | 261 | −64.954 | 59.105 | 59.457 | .00 | 69.28 | B |
| ATOM | 5182 | O | ASN | B | 261 | −65.784 | 59.322 | 60.339 | .00 | 69.28 | B |
| ATOM | 5183 | N | ALA | B | 262 | −65.277 | 58.814 | 58.207 | 1.00 | 70.42 | B |
| ATOM | 5184 | CA | ALA | B | 262 | −66.655 | 58.685 | 57.778 | 1.00 | 71.38 | B |
| ATOM | 5185 | CB | ALA | B | 262 | −66.760 | 59.065 | 56.318 | 1.00 | 70.35 | B |
| ATOM | 5186 | C | ALA | B | 262 | −67.180 | 57.256 | 58.001 | 1.00 | 72.65 | B |
| ATOM | 5187 | O | ALA | B | 262 | −67.353 | 56.493 | 57.043 | 1.00 | 71.88 | B |
| ATOM | 5188 | N | ARG | B | 263 | −67.426 | 56.910 | 59.270 | 1.00 | 74.22 | B |
| ATOM | 5189 | CA | ARG | B | 263 | −67.932 | 55.587 | 59.646 | 1.00 | 75.65 | B |
| ATOM | 5190 | CB | ARG | B | 263 | −67.798 | 55.373 | 61.167 | 1.00 | 76.58 | B |
| ATOM | 5191 | CG | ARG | B | 263 | −68.969 | 54.607 | 61.794 | 1.00 | 79.91 | B |
| ATOM | 5192 | CD | ARG | B | 263 | −68.655 | 54.068 | 63.181 | 1.00 | 81.03 | B |
| ATOM | 5193 | NE | ARG | B | 263 | −68.133 | 52.705 | 63.113 | 1.00 | 82.95 | B |
| ATOM | 5194 | CZ | ARG | B | 263 | −66.971 | 52.322 | 63.636 | 1.00 | 83.57 | B |
| ATOM | 5195 | NH1 | ARG | B | 263 | −66.200 | 53.207 | 64.266 | 1.00 | 82.34 | B |
| ATOM | 5196 | NH2 | ARG | B | 263 | −66.588 | 51.050 | 63.537 | 1.00 | 83.02 | B |
| ATOM | 5197 | C | ARG | B | 263 | −69.387 | 55.376 | 59.218 | 1.00 | 75.33 | B |
| ATOM | 5198 | O | ARG | B | 263 | −70.240 | 56.234 | 59.446 | 1.00 | 76.04 | B |
| ATOM | 5199 | N | VAL | B | 264 | −69.658 | 54.228 | 58.602 | 1.00 | 74.40 | B |
| ATOM | 5200 | CA | VAL | B | 264 | −70.999 | 53.890 | 58.148 | 1.00 | 73.60 | B |
| ATOM | 5201 | CB | VAL | B | 264 | −70.956 | 52.731 | 57.144 | 1.00 | 73.08 | B |
| ATOM | 5202 | CG1 | VAL | B | 264 | −72.365 | 52.260 | 56.840 | 1.00 | 72.89 | B |
| ATOM | 5203 | CG2 | VAL | B | 264 | −70.248 | 53.179 | 55.868 | 1.00 | 71.76 | B |
| ATOM | 5204 | C | VAL | B | 264 | −71.880 | 53.493 | 59.326 | 1.00 | 74.16 | B |
| ATOM | 5205 | O | VAL | B | 264 | −71.517 | 52.622 | 60.116 | 1.00 | 74.43 | B |
| ATOM | 5206 | N | LEU | B | 265 | −73.041 | 54.135 | 59.436 | 1.00 | 75.34 | B |
| ATOM | 5207 | CA | LEU | B | 265 | −73.984 | 53.859 | 60.523 | 1.00 | 76.28 | B |
| ATOM | 5208 | CB | LEU | B | 265 | −74.451 | 55.171 | 61.154 | 1.00 | 74.60 | B |
| ATOM | 5209 | CG | LEU | B | 265 | −73.315 | 56.051 | 61.681 | 1.00 | 74.93 | B |
| ATOM | 5210 | CD1 | LEU | B | 265 | −73.843 | 57.399 | 62.159 | 1.00 | 73.82 | B |
| ATOM | 5211 | CD2 | LEU | B | 265 | −72.621 | 55.317 | 62.808 | 1.00 | 74.69 | B |
| ATOM | 5212 | C | LEU | B | 265 | −75.198 | 53.060 | 60.049 | 1.00 | 77.14 | B |
| ATOM | 5213 | O | LEU | B | 265 | −75.590 | 52.063 | 60.674 | 1.00 | 75.95 | B |
| ATOM | 5214 | N | ALA | B | 266 | −75.782 | 53.495 | 58.938 | 1.00 | 78.33 | B |
| ATOM | 5215 | CA | ALA | B | 266 | −76.952 | 52.822 | 58.405 | 1.00 | 80.54 | B |
| ATOM | 5216 | CB | ALA | B | 266 | −78.206 | 53.615 | 58.777 | 1.00 | 79.90 | B |
| ATOM | 5217 | C | ALA | B | 266 | −76.907 | 52.587 | 56.894 | 1.00 | 82.12 | B |
| ATOM | 5218 | O | ALA | B | 266 | −76.253 | 53.314 | 56.143 | 1.00 | 82.15 | B |
| ATOM | 5219 | N | LEU | B | 267 | −77.603 | 51.542 | 56.466 | 1.00 | 83.96 | B |
| ATOM | 5220 | CA | LEU | B | 267 | −77.703 | 51.200 | 55.059 | 1.00 | 85.43 | B |
| ATOM | 5221 | CB | LEU | B | 267 | −77.122 | 49.811 | 54.783 | 1.00 | 84.65 | B |
| ATOM | 5222 | CG | LEU | B | 267 | −75.597 | 49.720 | 54.765 | 1.00 | 84.06 | B |
| ATOM | 5223 | CD1 | LEU | B | 267 | −75.164 | 48.291 | 54.432 | 1.00 | 83.71 | B |
| ATOM | 5224 | CD2 | LEU | B | 267 | −75.055 | 50.703 | 53.736 | 1.00 | 83.33 | B |
| ATOM | 5225 | C | LEU | B | 267 | −79.187 | 51.213 | 54.738 | 1.00 | 87.03 | B |
| ATOM | 5226 | O | LEU | B | 267 | −80.026 | 51.003 | 55.624 | 1.00 | 87.19 | B |
| ATOM | 5227 | N | SER | B | 268 | −79.508 | 51.469 | 53.475 | 1.00 | 88.39 | B |
| ATOM | 5228 | CA | SER | B | 268 | −80.893 | 51.519 | 53.042 | 1.00 | 89.27 | B |
| ATOM | 5229 | CB | SER | B | 268 | −81.494 | 52.888 | 53.376 | 1.00 | 90.38 | B |
| ATOM | 5230 | OG | SER | B | 268 | −81.167 | 53.291 | 54.700 | 1.00 | 92.24 | B |
| ATOM | 5231 | C | SER | B | 268 | −80.963 | 51.283 | 51.543 | 1.00 | 89.86 | B |
| ATOM | 5232 | O | SER | B | 268 | −80.301 | 51.968 | 50.763 | 1.00 | 88.54 | B |
| ATOM | 5233 | N | CYS | B | 269 | −81.755 | 50.298 | 51.143 | 1.00 | 91.48 | B |
| ATOM | 5234 | CA | CYS | B | 269 | −81.922 | 49.999 | 49.733 | 1.00 | 93.80 | B |
| ATOM | 5235 | CB | CYS | B | 269 | −81.411 | 48.595 | 49.425 | 1.00 | 93.96 | B |
| ATOM | 5236 | SG | CYS | B | 269 | −81.904 | 47.383 | 50.651 | 1.00 | 96.87 | B |
| ATOM | 5237 | C | CYS | B | 269 | −83.397 | 50.119 | 49.376 | 1.00 | 95.22 | B |
| ATOM | 5238 | O | CYS | B | 269 | −84.264 | 49.576 | 50.065 | 1.00 | 95.85 | B |
| ATOM | 5239 | N | SER | B | 270 | −83.671 | 50.860 | 48.308 | 1.00 | 96.16 | B |
| ATOM | 5240 | CA | SER | B | 270 | −85.032 | 51.066 | 47.836 | 1.00 | 96.30 | B |
| ATOM | 5241 | CB | SER | B | 270 | −85.308 | 52.560 | 47.658 | 1.00 | 96.76 | B |
| ATOM | 5242 | OG | SER | B | 270 | −84.383 | 53.148 | 46.756 | 1.00 | 96.65 | B |
| ATOM | 5243 | C | SER | B | 270 | −85.178 | 50.346 | 46.505 | 1.00 | 96.69 | B |
| ATOM | 5244 | O | SER | B | 270 | −84.253 | 50.331 | 45.693 | 1.00 | 96.58 | B |
| ATOM | 5245 | N | CYS | B | 271 | −86.337 | 49.744 | 46.283 | .00 | 96.99 | B |
| ATOM | 5246 | CA | CYS | B | 271 | −86.572 | 49.015 | 45.050 | .00 | 97.45 | B |
| ATOM | 5247 | CB | CYS | B | 271 | −86.878 | 47.554 | 45.383 | .00 | 97.93 | B |
| ATOM | 5248 | SG | CYS | B | 271 | −85.668 | 46.821 | 46.533 | 1.00 | 100.00 | B |
| ATOM | 5249 | C | CYS | B | 271 | −87.720 | 49.654 | 44.275 | .00 | 97.29 | B |
| ATOM | 5250 | O | CYS | B | 271 | −87.902 | 50.871 | 44.324 | .00 | 97.20 | B |
| ATOM | 5251 | N | ALA | B | 272 | −88.483 | 48.833 | 43.559 | .00 | 97.23 | B |
| ATOM | 5252 | CA | ALA | B | 272 | −89.620 | 49.306 | 42.772 | .00 | 97.21 | B |
| ATOM | 5253 | CB | ALA | B | 272 | −90.861 | 49.387 | 43.653 | .00 | 97.24 | B |
| ATOM | 5254 | C | ALA | B | 272 | −89.359 | 50.658 | 42.113 | .00 | 97.20 | B |
| ATOM | 5255 | O | ALA | B | 272 | −88.222 | 50.984 | 41.780 | .00 | 97.22 | B |
| ATOM | 5256 | N | ALA | B | 273 | −90.424 | 51.436 | 41.932 | .00 | 97.17 | B |
| ATOM | 5257 | CA | ALA | B | 273 | −90.348 | 52.758 | 41.313 | .00 | 97.11 | B |
| ATOM | 5258 | CB | ALA | B | 273 | −89.247 | 53.589 | 41.973 | .00 | 97.11 | B |
| ATOM | 5259 | C | ALA | B | 273 | −90.105 | 52.660 | 39.810 | .00 | 97.08 | B |
| ATOM | 5260 | O | ALA | B | 273 | −90.485 | 51.676 | 39.174 | .00 | 97.09 | B |
| ATOM | 5261 | N | ALA | B | 274 | −89.471 | 53.685 | 39.247 | .00 | 97.01 | B |
| ATOM | 5262 | CA | ALA | B | 274 | −89.182 | 53.721 | 37.818 | .00 | 96.94 | B |
| ATOM | 5263 | CB | ALA | B | 274 | −89.781 | 54.975 | 37.201 | .00 | 96.92 | B |
| ATOM | 5264 | C | ALA | B | 274 | −87.683 | 53.675 | 37.548 | .00 | 96.88 | B |
| ATOM | 5265 | O | ALA | B | 274 | −87.006 | 54.703 | 37.577 | .00 | 96.85 | B |
| ATOM | 5266 | N | ALA | B | 275 | −87.173 | 52.476 | 37.283 | .00 | 96.84 | B |
| ATOM | 5267 | CA | ALA | B | 275 | −85.755 | 52.285 | 37.004 | .00 | 96.85 | B |
| ATOM | 5268 | CB | ALA | B | 275 | −84.912 | 52.869 | 38.136 | .00 | 96.75 | B |
| ATOM | 5269 | C | ALA | B | 275 | −85.443 | 50.805 | 36.837 | .00 | 96.91 | B |
| ATOM | 5270 | O | ALA | B | 275 | −85.900 | 50.164 | 35.889 | .00 | 96.88 | B |
| ATOM | 5271 | N | ALA | B | 276 | −84.664 | 50.270 | 37.770 | 1.00 | 97.14 | B |
| ATOM | 5272 | CA | ALA | B | 276 | −84.276 | 48.861 | 37.741 | 1.00 | 97.25 | B |
| ATOM | 5273 | CB | ALA | B | 276 | −83.456 | 48.567 | 36.473 | 1.00 | 97.69 | B |
| ATOM | 5274 | C | ALA | B | 276 | −83.468 | 48.501 | 38.992 | 1.00 | 96.45 | B |
| ATOM | 5275 | O | ALA | B | 276 | −83.939 | 48.693 | 40.118 | 1.00 | 96.47 | B |
| ATOM | 5276 | N | ALA | B | 277 | −82.259 | 47.982 | 38.789 | .00 | 95.06 | B |
| ATOM | 5277 | CA | ALA | B | 277 | −81.386 | 47.601 | 39.896 | .00 | 93.75 | B |
| ATOM | 5278 | CB | ALA | B | 277 | −79.927 | 47.731 | 39.480 | .00 | 93.66 | B |
| ATOM | 5279 | C | ALA | B | 277 | −81.671 | 48.486 | 41.103 | .00 | 92.94 | B |
| ATOM | 5280 | O | ALA | B | 277 | −81.626 | 49.713 | 41.009 | .00 | 92.73 | B |
| ATOM | 5281 | N | ALA | B | 278 | −81.976 | 47.852 | 42.230 | .00 | 92.12 | B |
| ATOM | 5282 | CA | ALA | B | 278 | −82.299 | 48.560 | 43.462 | 1.00 | 91.66 | B |
| ATOM | 5283 | CB | ALA | B | 278 | −82.389 | 47.565 | 44.620 | 1.00 | 90.95 | B |
| ATOM | 5284 | C | ALA | B | 278 | −81.326 | 49.686 | 43.816 | 1.00 | 91.41 | B |
| ATOM | 5285 | O | ALA | B | 278 | −80.319 | 49.906 | 43.131 | 1.00 | 92.07 | B |
| ATOM | 5286 | N | SER | B | 279 | −81.643 | 50.392 | 44.898 | 1.00 | 90.54 | B |
| ATOM | 5287 | CA | SER | B | 279 | −80.827 | 51.500 | 45.373 | 1.00 | 89.92 | B |
| ATOM | 5288 | CB | SER | B | 279 | −81.717 | 52.690 | 45.757 | 1.00 | 90.64 | B |
| ATOM | 5289 | OG | SER | B | 279 | −80.976 | 53.722 | 46.395 | 1.00 | 90.56 | B |
| ATOM | 5290 | C | SER | B | 279 | −79.994 | 51.087 | 46.574 | .00 | 88.94 | B |
| ATOM | 5291 | O | SER | B | 279 | −80.035 | 49.939 | 47.016 | .00 | 88.87 | B |
| ATOM | 5292 | N | TRP | B | 280 | −79.243 | 52.047 | 47.094 | .00 | 87.78 | B |
| ATOM | 5293 | CA | TRP | B | 280 | −78.380 | 51.841 | 48.243 | 1.00 | 86.61 | B |
| ATOM | 5294 | CB | TRP | B | 280 | −77.141 | 51.043 | 47.832 | 1.00 | 85.87 | B |
| ATOM | 5295 | CG | TRP | B | 280 | −77.303 | 49.564 | 47.947 | 1.00 | 84.37 | B |
| ATOM | 5296 | CD2 | TRP | B | 280 | −77.763 | 48.840 | 49.092 | 1.00 | 83.72 | B |
| ATOM | 5297 | CE2 | TRP | B | 280 | −77.733 | 47.467 | 48.758 | 1.00 | 84.10 | B |
| ATOM | 5298 | CE3 | TRP | B | 280 | −78.198 | 49.219 | 50.370 | 1.00 | 84.35 | B |
| ATOM | 5299 | CD1 | TRP | B | 280 | −77.019 | 48.632 | 46.993 | 1.00 | 84.49 | B |
| ATOM | 5300 | NE1 | TRP | B | 280 | −77.273 | 47.368 | 47.472 | 1.00 | 84.48 | B |
| ATOM | 5301 | CZ2 | TRP | B | 280 | −78.123 | 46.468 | 49.654 | 1.00 | 84.35 | B |
| ATOM | 5302 | CZ3 | TRP | B | 280 | −78.586 | 48.228 | 51.265 | 1.00 | 84.57 | B |
| ATOM | 5303 | CH2 | TRP | B | 280 | −78.546 | 46.865 | 50.900 | 1.00 | 85.51 | B |
| ATOM | 5304 | C | TRP | B | 280 | −77.967 | 53.210 | 48.750 | 1.00 | 86.24 | B |
| ATOM | 5305 | O | TRP | B | 280 | −77.330 | 53.974 | 48.028 | 1.00 | 86.40 | B |
| ATOM | 5306 | N | SER | B | 281 | −78.338 | 53.529 | 49.982 | 1.00 | 85.63 | B |
| ATOM | 5307 | CA | SER | B | 281 | −77.987 | 54.828 | 50.544 | 1.00 | 86.80 | B |
| ATOM | 5308 | CB | SER | B | 281 | −79.240 | 55.680 | 50.775 | 1.00 | 86.95 | B |
| ATOM | 5309 | OG | SER | B | 281 | −80.043 | 55.137 | 51.813 | 1.00 | 87.67 | B |
| ATOM | 5310 | C | SER | B | 281 | −77.239 | 54.662 | 51.856 | 1.00 | 87.15 | B |
| ATOM | 5311 | O | SER | B | 281 | −77.681 | 53.932 | 52.754 | 1.00 | 87.21 | B |
| ATOM | 5312 | N | ILE | B | 282 | −76.114 | 55.358 | 51.978 | 1.00 | 86.99 | B |
| ATOM | 5313 | CA | ILE | B | 282 | −75.315 | 55.247 | 53.184 | 1.00 | 87.01 | B |
| ATOM | 5314 | CB | ILE | B | 282 | −73.849 | 54.960 | 52.815 | 1.00 | 86.78 | B |
| ATOM | 5315 | CG2 | ILE | B | 282 | −73.007 | 54.814 | 54.071 | 1.00 | 85.95 | B |
| ATOM | 5316 | CG1 | ILE | B | 282 | −73.786 | 53.668 | 51.998 | 1.00 | 87.30 | B |
| ATOM | 5317 | CD1 | ILE | B | 282 | −72.394 | 53.265 | 51.571 | 1.00 | 89.34 | B |
| ATOM | 5318 | C | ILE | B | 282 | −75.388 | 56.446 | 54.122 | 1.00 | 86.79 | B |
| ATOM | 5319 | O | ILE | B | 282 | −75.288 | 57.594 | 53.698 | 1.00 | 86.12 | B |
| ATOM | 5320 | N | ILE | B | 283 | −75.579 | 56.157 | 55.404 | 1.00 | 87.00 | B |
| ATOM | 5321 | CA | ILE | B | 283 | −75.647 | 57.194 | 56.421 | 1.00 | 88.37 | B |
| ATOM | 5322 | CB | ILE | B | 283 | −76.889 | 57.041 | 57.346 | 1.00 | 88.79 | B |
| ATOM | 5323 | CG2 | ILE | B | 283 | −77.056 | 58.294 | 58.199 | 1.00 | 87.36 | B |
| ATOM | 5324 | CG1 | ILE | B | 283 | −78.157 | 56.812 | 56.521 | 1.00 | 89.71 | B |
| ATOM | 5325 | CD1 | ILE | B | 283 | −78.292 | 55.397 | 55.949 | 1.00 | 90.93 | B |
| ATOM | 5326 | C | ILE | B | 283 | −74.399 | 57.053 | 57.288 | 1.00 | 89.39 | B |
| ATOM | 5327 | O | ILE | B | 283 | −74.383 | 56.270 | 58.243 | 1.00 | 90.24 | B |
| ATOM | 5328 | N | SER | B | 284 | −73.349 | 57.794 | 56.941 | 1.00 | 89.62 | B |
| ATOM | 5329 | CA | SER | B | 284 | −72.102 | 57.760 | 57.701 | 1.00 | 89.19 | B |
| ATOM | 5330 | CB | SER | B | 284 | −70.888 | 57.677 | 56.766 | 1.00 | 88.83 | B |
| ATOM | 5331 | OG | SER | B | 284 | −70.810 | 58.802 | 55.908 | 1.00 | 88.57 | B |
| ATOM | 5332 | C | SER | B | 284 | −72.022 | 59.022 | 58.535 | 1.00 | 89.19 | B |
| ATOM | 5333 | O | SER | B | 284 | −72.520 | 60.070 | 58.130 | 1.00 | 89.15 | B |
| ATOM | 5334 | N | ALA | B | 285 | −71.398 | 58.922 | 59.700 | 1.00 | 89.89 | B |
| ATOM | 5335 | CA | ALA | B | 285 | −71.278 | 60.070 | 60.585 | 1.00 | 90.95 | B |
| ATOM | 5336 | CB | ALA | B | 285 | −71.154 | 59.596 | 62.033 | 1.00 | 90.98 | B |
| ATOM | 5337 | C | ALA | B | 285 | −70.091 | 60.958 | 60.220 | 1.00 | 91.75 | B |
| ATOM | 5338 | O | ALA | B | 285 | −69.453 | 60.778 | 59.181 | 1.00 | 91.23 | B |
| ATOM | 5339 | N | SER | B | 286 | −69.817 | 61.923 | 61.093 | 1.00 | 93.48 | B |
| ATOM | 5340 | CA | SER | B | 286 | −68.714 | 62.867 | 60.933 | 1.00 | 94.13 | B |
| ATOM | 5341 | CB | SER | B | 286 | −69.026 | 63.877 | 59.826 | 1.00 | 93.67 | B |
| ATOM | 5342 | OG | SER | B | 286 | −68.016 | 64.869 | 59.759 | 1.00 | 93.66 | B |
| ATOM | 5343 | C | SER | B | 286 | −68.505 | 63.606 | 62.260 | 1.00 | 94.95 | B |
| ATOM | 5344 | O | SER | B | 286 | −69.405 | 63.646 | 63.114 | 1.00 | 95.13 | B |
| ATOM | 5345 | N | ALA | B | 287 | −67.318 | 64.184 | 62.433 | .50 | 94.66 | B |
| ATOM | 5346 | CA | ALA | B | 287 | −66.994 | 64.920 | 63.654 | .50 | 94.15 | B |
| ATOM | 5347 | CB | ALA | B | 287 | −65.839 | 64.239 | 64.382 | .50 | 94.14 | B |
| ATOM | 5348 | C | ALA | B | 287 | −66.632 | 66.366 | 63.326 | .50 | 93.61 | B |
| ATOM | 5349 | O | ALA | B | 287 | −66.838 | 67.271 | 64.137 | .50 | 93.03 | B |
| ATOM | 5350 | N | ALA | B | 288 | −66.087 | 66.568 | 62.131 | .50 | 93.24 | B |
| ATOM | 5351 | CA | ALA | B | 288 | −65.701 | 67.894 | 61.672 | .50 | 93.05 | B |
| ATOM | 5352 | CB | ALA | B | 288 | −64.388 | 67.821 | 60.899 | .50 | 92.42 | B |
| ATOM | 5353 | C | ALA | B | 288 | −66.808 | 68.435 | 60.777 | .50 | 93.32 | B |
| ATOM | 5354 | O | ALA | B | 288 | −66.632 | 69.446 | 60.095 | .50 | 93.12 | B |
| ATOM | 5355 | N | ALA | B | 289 | −67.948 | 67.746 | 60.784 | .50 | 93.58 | B |
| ATOM | 5356 | CA | ALA | B | 289 | −69.098 | 68.144 | 59.975 | .50 | 93.68 | B |
| ATOM | 5357 | CB | ALA | B | 289 | −69.202 | 67.252 | 58.739 | .50 | 93.48 | B |
| ATOM | 5358 | C | ALA | B | 289 | −70.400 | 68.084 | 60.774 | .50 | 93.61 | B |
| ATOM | 5359 | O | ALA | B | 289 | −71.408 | 68.667 | 60.372 | .50 | 93.24 | B |
| ATOM | 5360 | N | ALA | B | 290 | −70.371 | 67.376 | 61.901 | .50 | 93.65 | B |
| ATOM | 5361 | CA | ALA | B | 290 | −71.542 | 67.236 | 62.767 | .50 | 93.47 | B |
| ATOM | 5362 | CB | ALA | B | 290 | −71.963 | 68.604 | 63.309 | .50 | 92.92 | B |
| ATOM | 5363 | C | ALA | B | 290 | −72.714 | 66.577 | 62.048 | .50 | 93.49 | B |
| ATOM | 5364 | O | ALA | B | 290 | −72.685 | 66.383 | 60.831 | .50 | 93.47 | B |
| ATOM | 5365 | N | ALA | B | 291 | −73.746 | 66.234 | 62.811 | .50 | 93.69 | B |
| ATOM | 5366 | CA | ALA | B | 291 | −74.933 | 65.593 | 62.258 | .50 | 94.19 | B |
| ATOM | 5367 | CB | ALA | B | 291 | −75.636 | 66.539 | 61.288 | .00 | 94.07 | B |
| ATOM | 5368 | C | ALA | B | 291 | −74.564 | 64.291 | 61.551 | .00 | 94.62 | B |
| ATOM | 5369 | O | ALA | B | 291 | −74.043 | 63.363 | 62.172 | .00 | 94.60 | B |
| ATOM | 5370 | N | ALA | B | 292 | −74.837 | 64.229 | 60.252 | 1.00 | 95.15 | B |
| ATOM | 5371 | CA | ALA | B | 292 | −74.538 | 63.045 | 59.450 | 1.00 | 96.05 | B |
| ATOM | 5372 | CB | ALA | B | 292 | −75.574 | 61.943 | 59.721 | 1.00 | 94.76 | B |
| ATOM | 5373 | C | ALA | B | 292 | −74.536 | 63.414 | 57.968 | 1.00 | 96.54 | B |
| ATOM | 5374 | O | ALA | B | 292 | −74.268 | 64.563 | 57.610 | 1.00 | 96.49 | B |
| ATOM | 5375 | N | GLU | B | 293 | −74.838 | 62.434 | 57.118 | 1.00 | 97.41 | B |
| ATOM | 5376 | CA | GLU | B | 293 | −74.883 | 62.632 | 55.670 | 1.00 | 98.33 | B |
| ATOM | 5377 | CB | GLU | B | 293 | −73.507 | 63.040 | 55.143 | 1.00 | 99.04 | B |
| ATOM | 5378 | CG | GLU | B | 293 | −72.366 | 62.190 | 55.669 | 1.00 | 99.49 | B |
| ATOM | 5379 | CD | GLU | B | 293 | −71.061 | 62.488 | 54.960 | 1.00 | 100.00 | B |
| ATOM | 5380 | OE1 | GLU | B | 293 | −70.831 | 63.675 | 54.636 | 1.00 | 100.00 | B |
| ATOM | 5381 | OE2 | GLU | B | 293 | −70.266 | 61.543 | 54.734 | 1.00 | 100.00 | B |
| ATOM | 5382 | C | GLU | B | 293 | −75.336 | 61.369 | 54.944 | 1.00 | 98.90 | B |
| ATOM | 5383 | O | GLU | B | 293 | −75.259 | 60.261 | 55.491 | 1.00 | 99.23 | B |
| ATOM | 5384 | N | GLU | B | 294 | −75.809 | 61.536 | 53.710 | 1.00 | 98.49 | B |
| ATOM | 5385 | CA | GLU | B | 294 | −76.260 | 60.396 | 52.922 | 1.00 | 98.38 | B |
| ATOM | 5386 | CB | GLU | B | 294 | −77.790 | 60.292 | 52.952 | 1.00 | 98.54 | B |
| ATOM | 5387 | CG | GLU | B | 294 | −78.325 | 59.078 | 52.198 | 1.00 | 99.63 | B |
| ATOM | 5388 | CD | GLU | B | 294 | −79.806 | 58.846 | 52.415 | 1.00 | 100.00 | B |
| ATOM | 5389 | OE1 | GLU | B | 294 | −80.624 | 59.677 | 51.956 | 1.00 | 98.50 | B |
| ATOM | 5390 | OE2 | GLU | B | 294 | −80.145 | 57.824 | 53.053 | 1.00 | 100.00 | B |
| ATOM | 5391 | C | GLU | B | 294 | −75.779 | 60.455 | 51.473 | 1.00 | 97.73 | B |
| ATOM | 5392 | O | GLU | B | 294 | −75.289 | 61.486 | 51.013 | 1.00 | 97.81 | B |
| ATOM | 5393 | N | GLU | B | 295 | −75.915 | 59.328 | 50.776 | 1.00 | 96.91 | B |
| ATOM | 5394 | CA | GLU | B | 295 | −75.527 | 59.193 | 49.373 | 1.00 | 95.48 | B |
| ATOM | 5395 | CB | GLU | B | 295 | −74.003 | 59.115 | 49.239 | 1.00 | 96.14 | B |
| ATOM | 5396 | CG | GLU | B | 295 | −73.262 | 60.402 | 49.559 | 1.00 | 95.41 | B |
| ATOM | 5397 | CD | GLU | B | 295 | −71.777 | 60.292 | 49.298 | .00 | 95.37 | B |
| ATOM | 5398 | OE1 | GLU | B | 295 | −71.394 | 60.039 | 48.137 | .00 | 95.25 | B |
| ATOM | 5399 | OE2 | GLU | B | 295 | −70.993 | 60.456 | 50.253 | .00 | 95.27 | B |
| ATOM | 5400 | C | GLU | B | 295 | −76.157 | 57.914 | 48.809 | 1.00 | 94.18 | B |
| ATOM | 5401 | O | GLU | B | 295 | −76.381 | 56.954 | 49.549 | 1.00 | 94.41 | B |
| ATOM | 5402 | N | ALA | B | 296 | −76.431 | 57.903 | 47.505 | 1.00 | 91.99 | B |
| ATOM | 5403 | CA | ALA | B | 296 | −77.046 | 56.746 | 46.851 | 1.00 | 90.31 | B |
| ATOM | 5404 | CB | ALA | B | 296 | −78.268 | 57.198 | 46.060 | 1.00 | 89.77 | B |
| ATOM | 5405 | C | ALA | B | 296 | −76.079 | 55.994 | 45.929 | 1.00 | 88.91 | B |
| ATOM | 5406 | O | ALA | B | 296 | −75.336 | 56.616 | 45.168 | 1.00 | 89.59 | B |
| ATOM | 5407 | N | PHE | B | 297 | −76.099 | 54.661 | 45.990 | 1.00 | 86.72 | B |
| ATOM | 5408 | CA | PHE | B | 297 | −75.217 | 53.830 | 45.159 | 1.00 | 85.03 | B |
| ATOM | 5409 | CB | PHE | B | 297 | −74.080 | 53.245 | 46.011 | 1.00 | 83.95 | B |
| ATOM | 5410 | CG | PHE | B | 297 | −73.258 | 54.283 | 46.712 | 1.00 | 82.09 | B |
| ATOM | 5411 | CD1 | PHE | B | 297 | −73.698 | 54.841 | 47.906 | 1.00 | 80.79 | B |
| ATOM | 5412 | CD2 | PHE | B | 297 | −72.074 | 54.752 | 46.140 | 1.00 | 80.96 | B |
| ATOM | 5413 | CE1 | PHE | B | 297 | −72.975 | 55.858 | 48.522 | 1.00 | 81.60 | B |
| ATOM | 5414 | CE2 | PHE | B | 297 | −71.343 | 55.770 | 46.745 | 1.00 | 81.23 | B |
| ATOM | 5415 | CZ | PHE | B | 297 | −71.792 | 56.328 | 47.940 | 1.00 | 81.14 | B |
| ATOM | 5416 | C | PHE | B | 297 | −75.946 | 52.684 | 44.454 | 1.00 | 83.99 | B |
| ATOM | 5417 | O | PHE | B | 297 | −77.124 | 52.444 | 44.708 | 1.00 | 84.92 | B |
| ATOM | 5418 | N | ASP | B | 298 | −75.245 | 51.976 | 43.572 | 1.00 | 82.75 | B |
| ATOM | 5419 | CA | ASP | B | 298 | −75.845 | 50.847 | 42.857 | 1.00 | 82.93 | B |
| ATOM | 5420 | CB | ASP | B | 298 | −75.458 | 50.864 | 41.376 | 1.00 | 84.95 | B |
| ATOM | 5421 | CG | ASP | B | 298 | −75.893 | 52.136 | 40.675 | 1.00 | 87.41 | B |
| ATOM | 5422 | OD1 | ASP | B | 298 | −77.043 | 52.584 | 40.910 | 1.00 | 88.06 | B |
| ATOM | 5423 | OD2 | ASP | B | 298 | −75.087 | 52.681 | 39.884 | 1.00 | 88.05 | B |
| ATOM | 5424 | C | ASP | B | 298 | −75.439 | 49.499 | 43.453 | 1.00 | 81.72 | B |
| ATOM | 5425 | O | ASP | B | 298 | −76.175 | 48.508 | 43.349 | 1.00 | 82.89 | B |
| ATOM | 5426 | N | ALA | B | 299 | −74.256 | 49.461 | 44.057 | 1.00 | 78.72 | B |
| ATOM | 5427 | CA | ALA | B | 299 | −73.752 | 48.245 | 44.681 | 1.00 | 75.39 | B |
| ATOM | 5428 | CB | ALA | B | 299 | −72.843 | 47.498 | 43.725 | 1.00 | 74.76 | B |
| ATOM | 5429 | C | ALA | B | 299 | −72.990 | 48.662 | 45.932 | 1.00 | 73.47 | B |
| ATOM | 5430 | O | ALA | B | 299 | −72.572 | 49.813 | 46.056 | 1.00 | 72.95 | B |
| ATOM | 5431 | N | VAL | B | 300 | −72.813 | 47.727 | 46.855 | 1.00 | 70.56 | B |
| ATOM | 5432 | CA | VAL | B | 300 | −72.128 | 48.019 | 48.100 | 1.00 | 68.63 | B |
| ATOM | 5433 | CB | VAL | B | 300 | −73.148 | 48.349 | 49.202 | 1.00 | 66.87 | B |
| ATOM | 5434 | CG1 | VAL | B | 300 | −72.480 | 48.342 | 50.561 | 1.00 | 67.75 | B |
| ATOM | 5435 | CG2 | VAL | B | 300 | −73.772 | 49.703 | 48.929 | 1.00 | 64.56 | B |
| ATOM | 5436 | C | VAL | B | 300 | −71.286 | 46.837 | 48.541 | 1.00 | 69.59 | B |
| ATOM | 5437 | O | VAL | B | 300 | −71.819 | 45.753 | 48.774 | 1.00 | 70.90 | B |
| ATOM | 5438 | N | ILE | B | 301 | −69.972 | 47.029 | 48.646 | 1.00 | 70.09 | B |
| ATOM | 5439 | CA | ILE | B | 301 | −69.108 | 45.938 | 49.089 | 1.00 | 69.40 | B |
| ATOM | 5440 | CB | ILE | B | 301 | −67.801 | 45.855 | 48.274 | 1.00 | 67.86 | B |
| ATOM | 5441 | CG2 | ILE | B | 301 | −66.877 | 44.810 | 48.884 | 1.00 | 66.63 | B |
| ATOM | 5442 | CG1 | ILE | B | 301 | −68.125 | 45.467 | 46.828 | 1.00 | 66.08 | B |
| ATOM | 5443 | CD1 | ILE | B | 301 | −66.916 | 45.352 | 45.915 | 1.00 | 65.83 | B |
| ATOM | 5444 | C | ILE | B | 301 | −68.796 | 46.111 | 50.566 | 1.00 | 69.94 | B |
| ATOM | 5445 | O | ILE | B | 301 | −68.222 | 47.108 | 50.990 | 1.00 | 69.26 | B |
| ATOM | 5446 | N | MSE | B | 302 | −69.229 | 45.131 | 51.344 | 1.00 | 72.09 | B |
| ATOM | 5447 | CA | MSE | B | 302 | −69.038 | 45.127 | 52.781 | 1.00 | 74.25 | B |
| ATOM | 5448 | CB | MSE | B | 302 | −70.063 | 44.188 | 53.416 | 1.00 | 79.03 | B |
| ATOM | 5449 | CG | MSE | B | 302 | −70.155 | 44.310 | 54.909 | 1.00 | 86.85 | B |
| ATOM | 5450 | SE | MSE | B | 302 | −70.749 | 46.072 | 55.384 | 1.00 | 100.00 | B |
| ATOM | 5451 | CE | MSE | B | 302 | −72.659 | 45.749 | 55.339 | 1.00 | 95.16 | B |
| ATOM | 5452 | C | MSE | B | 302 | −67.623 | 44.628 | 53.040 | 1.00 | 72.94 | B |
| ATOM | 5453 | O | MSE | B | 302 | −67.181 | 43.664 | 52.416 | 1.00 | 73.96 | B |
| ATOM | 5454 | N | THR | B | 303 | −66.906 | 45.283 | 53.946 | 1.00 | 70.83 | B |
| ATOM | 5455 | CA | THR | B | 303 | −65.537 | 44.876 | 54.248 | 1.00 | 69.18 | B |
| ATOM | 5456 | CB | THR | B | 303 | −64.519 | 45.713 | 53.414 | 1.00 | 68.98 | B |
| ATOM | 5457 | OG1 | THR | B | 303 | −64.436 | 45.168 | 52.091 | 1.00 | 68.29 | B |
| ATOM | 5458 | CG2 | THR | B | 303 | −63.136 | 45.692 | 54.037 | 1.00 | 69.49 | B |
| ATOM | 5459 | C | THR | B | 303 | −65.221 | 44.952 | 55.739 | 1.00 | 67.78 | B |
| ATOM | 5460 | O | THR | B | 303 | −64.219 | 44.410 | 56.197 | 1.00 | 66.96 | B |
| ATOM | 5461 | N | ALA | B | 304 | −66.089 | 45.603 | 56.503 | 1.00 | 66.99 | B |
| ATOM | 5462 | CA | ALA | B | 304 | −65.875 | 45.699 | 57.936 | 1.00 | 67.16 | B |
| ATOM | 5463 | CB | ALA | B | 304 | −66.845 | 46.681 | 58.544 | 1.00 | 65.82 | B |
| ATOM | 5464 | C | ALA | B | 304 | −66.087 | 44.318 | 58.532 | 1.00 | 68.90 | B |
| ATOM | 5465 | O | ALA | B | 304 | −66.710 | 43.450 | 57.914 | 1.00 | 69.64 | B |
| ATOM | 5466 | N | PRO | B | 305 | −65.553 | 44.081 | 59.735 | 1.00 | 70.72 | B |
| ATOM | 5467 | CD | PRO | B | 305 | −64.526 | 44.872 | 60.431 | 1.00 | 71.10 | B |
| ATOM | 5468 | CA | PRO | B | 305 | −65.720 | 42.770 | 60.373 | 1.00 | 72.13 | B |
| ATOM | 5469 | CB | PRO | B | 305 | −64.869 | 42.893 | 61.634 | 1.00 | 72.14 | B |
| ATOM | 5470 | CG | PRO | B | 305 | −63.757 | 43.793 | 61.176 | 1.00 | 72.33 | B |
| ATOM | 5471 | C | PRO | B | 305 | −67.188 | 42.483 | 60.685 | 1.00 | 73.38 | B |
| ATOM | 5472 | O | PRO | B | 305 | −67.980 | 43.409 | 60.911 | 1.00 | 72.46 | B |
| ATOM | 5473 | N | LEU | B | 306 | −67.537 | 41.196 | 60.703 | 1.00 | 74.48 | B |
| ATOM | 5474 | CA | LEU | B | 306 | −68.906 | 40.764 | 60.967 | 1.00 | 74.86 | B |
| ATOM | 5475 | CB | LEU | B | 306 | −68.989 | 39.230 | 60.939 | 1.00 | 73.29 | B |
| ATOM | 5476 | CG | LEU | B | 306 | −70.200 | 38.629 | 60.221 | .00 | 72.44 | B |
| ATOM | 5477 | CD1 | LEU | B | 306 | −70.516 | 39.427 | 58.964 | .00 | 71.81 | B |
| ATOM | 5478 | CD2 | LEU | B | 306 | −69.910 | 37.179 | 59.874 | .00 | 71.87 | B |
| ATOM | 5479 | C | LEU | B | 306 | −69.452 | 41.306 | 62.288 | 1.00 | 75.27 | B |
| ATOM | 5480 | O | LEU | B | 306 | −70.658 | 41.486 | 62.429 | 1.00 | 76.16 | B |
| ATOM | 5481 | N | CYS | B | 307 | −68.574 | 41.586 | 63.244 | 1.00 | 75.45 | B |
| ATOM | 5482 | CA | CYS | B | 307 | −69.019 | 42.108 | 64.528 | 1.00 | 76.65 | B |
| ATOM | 5483 | CB | CYS | B | 307 | −67.928 | 41.963 | 65.586 | 1.00 | 76.82 | B |
| ATOM | 5484 | SG | CYS | B | 307 | −66.678 | 43.275 | 65.535 | 1.00 | 77.01 | B |
| ATOM | 5485 | C | CYS | B | 307 | −69.376 | 43.578 | 64.394 | 1.00 | 77.84 | B |
| ATOM | 5486 | O | CYS | B | 307 | −69.962 | 44.166 | 65.298 | 1.00 | 79.20 | B |
| ATOM | 5487 | N | ASP | B | 308 | −68.996 | 44.184 | 63.279 | 1.00 | 79.18 | B |
| ATOM | 5488 | CA | ASP | B | 308 | −69.315 | 45.588 | 63.057 | 1.00 | 81.36 | B |
| ATOM | 5489 | CB | ASP | B | 308 | −68.143 | 46.319 | 62.402 | 1.00 | 82.46 | B |
| ATOM | 5490 | CG | ASP | B | 308 | −67.156 | 46.864 | 63.422 | 1.00 | 83.43 | B |
| ATOM | 5491 | OD1 | ASP | B | 308 | −67.471 | 46.816 | 64.634 | 1.00 | 83.86 | B |
| ATOM | 5492 | OD2 | ASP | B | 308 | −66.075 | 47.349 | 63.013 | 1.00 | 83.48 | B |
| ATOM | 5493 | C | ASP | B | 308 | −70.552 | 45.719 | 62.184 | 1.00 | 82.86 | B |
| ATOM | 5494 | O | ASP | B | 308 | −71.277 | 46.709 | 62.269 | 1.00 | 82.75 | B |
| ATOM | 5495 | N | VAL | B | 309 | −70.784 | 44.714 | 61.340 | 1.00 | 84.47 | B |
| ATOM | 5496 | CA | VAL | B | 309 | −71.947 | 44.696 | 60.462 | 1.00 | 85.28 | B |
| ATOM | 5497 | CB | VAL | B | 309 | −71.927 | 43.452 | 59.527 | 1.00 | 84.56 | B |
| ATOM | 5498 | CG1 | VAL | B | 309 | −73.102 | 43.494 | 58.566 | 1.00 | 84.64 | B |
| ATOM | 5499 | CG2 | VAL | B | 309 | −70.635 | 43.415 | 58.734 | 1.00 | 84.12 | B |
| ATOM | 5500 | C | VAL | B | 309 | −73.192 | 44.657 | 61.356 | 1.00 | 86.86 | B |
| ATOM | 5501 | O | VAL | B | 309 | −74.221 | 45.254 | 61.037 | 1.00 | 86.64 | B |
| ATOM | 5502 | N | LYS | B | 310 | −73.077 | 43.975 | 62.492 | 1.00 | 88.48 | B |
| ATOM | 5503 | CA | LYS | B | 310 | −74.189 | 43.860 | 63.430 | 1.00 | 90.13 | B |
| ATOM | 5504 | CB | LYS | B | 310 | −73.815 | 42.933 | 64.603 | 1.00 | 91.20 | B |
| ATOM | 5505 | CG | LYS | B | 310 | −73.147 | 43.630 | 65.802 | 1.00 | 93.52 | B |
| ATOM | 5506 | CD | LYS | B | 310 | −74.165 | 44.373 | 66.693 | 1.00 | 94.14 | B |
| ATOM | 5507 | CE | LYS | B | 310 | −73.472 | 45.216 | 67.761 | 1.00 | 94.40 | B |
| ATOM | 5508 | NZ | LYS | B | 310 | −74.446 | 45.938 | 68.628 | 1.00 | 93.65 | B |
| ATOM | 5509 | C | LYS | B | 310 | −74.624 | 45.226 | 63.966 | 1.00 | 90.36 | B |
| ATOM | 5510 | O | LYS | B | 310 | −75.822 | 45.512 | 64.050 | 1.00 | 91.58 | B |
| ATOM | 5511 | N | SER | B | 311 | −73.658 | 46.062 | 64.339 | 1.00 | 90.09 | B |
| ATOM | 5512 | CA | SER | B | 311 | −73.967 | 47.387 | 64.876 | 1.00 | 90.65 | B |
| ATOM | 5513 | CB | SER | B | 311 | −72.802 | 47.911 | 65.723 | 1.00 | 90.17 | B |
| ATOM | 5514 | OG | SER | B | 311 | −71.587 | 47.894 | 64.990 | 1.00 | 91.57 | B |
| ATOM | 5515 | C | SER | B | 311 | −74.282 | 48.378 | 63.762 | 1.00 | 90.78 | B |
| ATOM | 5516 | O | SER | B | 311 | −74.012 | 49.575 | 63.883 | 1.00 | 89.36 | B |
| ATOM | 5517 | N | MSE | B | 312 | −74.859 | 47.858 | 62.682 | 1.00 | 91.65 | B |
| ATOM | 5518 | CA | MSE | B | 312 | −75.233 | 48.662 | 61.525 | 1.00 | 93.59 | B |
| ATOM | 5519 | CB | MSE | B | 312 | −74.490 | 48.176 | 60.275 | 1.00 | 94.67 | B |
| ATOM | 5520 | CG | MSE | B | 312 | −73.166 | 48.865 | 59.983 | 1.00 | 96.17 | B |
| ATOM | 5521 | SE | MSE | B | 312 | −72.479 | 48.377 | 58.213 | 1.00 | 100.00 | B |
| ATOM | 5522 | CE | MSE | B | 312 | −70.924 | 47.351 | 58.746 | 1.00 | 97.01 | B |
| ATOM | 5523 | C | MSE | B | 312 | −76.742 | 48.625 | 61.244 | 1.00 | 93.60 | B |
| ATOM | 5524 | O | MSE | B | 312 | −77.280 | 47.596 | 60.819 | 1.00 | 93.23 | B |
| ATOM | 5525 | N | LYS | B | 313 | −77.414 | 49.752 | 61.470 | 1.00 | 93.40 | B |
| ATOM | 5526 | CA | LYS | B | 313 | −78.846 | 49.843 | 61.215 | 1.00 | 93.21 | B |
| ATOM | 5527 | CB | LYS | B | 313 | −79.388 | 51.181 | 61.715 | 1.00 | 91.96 | B |
| ATOM | 5528 | CG | LYS | B | 313 | −79.428 | 51.300 | 63.224 | 1.00 | 90.80 | B |
| ATOM | 5529 | CD | LYS | B | 313 | −80.205 | 52.538 | 63.641 | 1.00 | 90.47 | B |
| ATOM | 5530 | CE | LYS | B | 313 | −80.465 | 52.558 | 65.143 | 1.00 | 90.43 | B |
| ATOM | 5531 | NZ | LYS | B | 313 | −81.221 | 53.779 | 65.561 | 1.00 | 89.39 | B |
| ATOM | 5532 | C | LYS | B | 313 | −79.124 | 49.695 | 59.721 | 1.00 | 93.82 | B |
| ATOM | 5533 | O | LYS | B | 313 | −79.032 | 50.659 | 58.962 | 1.00 | 93.77 | B |
| ATOM | 5534 | N | ILE | B | 314 | −79.465 | 48.478 | 59.310 | 1.00 | 94.75 | B |
| ATOM | 5535 | CA | ILE | B | 314 | −79.747 | 48.189 | 57.908 | 1.00 | 95.99 | B |
| ATOM | 5536 | CB | ILE | B | 314 | −78.763 | 47.079 | 57.396 | 1.00 | 96.07 | B |
| ATOM | 5537 | CG2 | ILE | B | 314 | −78.821 | 45.863 | 58.307 | 1.00 | 96.05 | B |
| ATOM | 5538 | CG1 | ILE | B | 314 | −79.073 | 46.703 | 55.947 | 1.00 | 95.91 | B |
| ATOM | 5539 | CD1 | ILE | B | 314 | −78.062 | 45.751 | 55.355 | 1.00 | 95.00 | B |
| ATOM | 5540 | C | ILE | B | 314 | −81.227 | 47.792 | 57.708 | 1.00 | 96.17 | B |
| ATOM | 5541 | O | ILE | B | 314 | −81.808 | 47.072 | 58.528 | 1.00 | 95.71 | B |
| ATOM | 5542 | N | ALA | B | 315 | −81.832 | 48.283 | 56.625 | 1.00 | 96.28 | B |
| ATOM | 5543 | CA | ALA | B | 315 | −83.239 | 48.007 | 56.330 | 1.00 | 96.71 | B |
| ATOM | 5544 | CB | ALA | B | 315 | −84.084 | 49.223 | 56.719 | 1.00 | 96.14 | B |
| ATOM | 5545 | C | ALA | B | 315 | −83.492 | 47.642 | 54.862 | 1.00 | 97.06 | B |
| ATOM | 5546 | O | ALA | B | 315 | −82.762 | 48.081 | 53.969 | 1.00 | 97.22 | B |
| ATOM | 5547 | N | LYS | B | 316 | −84.535 | 46.846 | 54.616 | 1.00 | 97.16 | B |
| ATOM | 5548 | CA | LYS | B | 316 | −84.882 | 46.430 | 53.256 | 1.00 | 97.39 | B |
| ATOM | 5549 | CB | LYS | B | 316 | −85.626 | 45.090 | 53.290 | .00 | 97.00 | B |
| ATOM | 5550 | CG | LYS | B | 316 | −86.923 | 45.104 | 54.087 | .00 | 96.63 | B |
| ATOM | 5551 | CD | LYS | B | 316 | −87.461 | 43.700 | 54.304 | .00 | 96.32 | B |
| ATOM | 5552 | CE | LYS | B | 316 | −86.533 | 42.898 | 55.202 | .00 | 96.12 | B |
| ATOM | 5553 | NZ | LYS | B | 316 | −87.067 | 41.544 | 55.506 | .00 | 95.95 | B |
| ATOM | 5554 | C | LYS | B | 316 | −85.725 | 47.487 | 52.535 | 1.00 | 97.91 | B |
| ATOM | 5555 | O | LYS | B | 316 | −85.232 | 48.578 | 52.235 | 1.00 | 98.46 | B |
| ATOM | 5556 | N | ARG | B | 317 | −86.989 | 47.166 | 52.255 | 1.00 | 98.48 | B |
| ATOM | 5557 | CA | ARG | B | 317 | −87.896 | 48.093 | 51.568 | 1.00 | 98.23 | B |
| ATOM | 5558 | CB | ARG | B | 317 | −89.243 | 47.405 | 51.287 | 1.00 | 98.27 | B |
| ATOM | 5559 | CG | ARG | B | 317 | −90.287 | 48.279 | 50.577 | .00 | 98.66 | B |
| ATOM | 5560 | CD | ARG | B | 317 | −91.125 | 49.082 | 51.566 | .00 | 99.00 | B |
| ATOM | 5561 | NE | ARG | B | 317 | −92.193 | 49.852 | 50.928 | 1.00 | 100.00 | B |
| ATOM | 5562 | CZ | ARG | B | 317 | −93.159 | 50.471 | 51.599 | 1.00 | 99.45 | B |
| ATOM | 5563 | NH1 | ARG | B | 317 | −93.179 | 50.405 | 52.924 | 1.00 | 100.00 | B |
| ATOM | 5564 | NH2 | ARG | B | 317 | −94.102 | 51.146 | 50.952 | 1.00 | 98.56 | B |
| ATOM | 5565 | C | ARG | B | 317 | −88.118 | 49.349 | 52.403 | 1.00 | 97.52 | B |
| ATOM | 5566 | O | ARG | B | 317 | −87.522 | 50.397 | 52.144 | 1.00 | 97.50 | B |
| ATOM | 5567 | N | GLY | B | 318 | −88.983 | 49.231 | 53.402 | 1.00 | 96.59 | B |
| ATOM | 5568 | CA | GLY | B | 318 | −89.272 | 50.351 | 54.277 | 1.00 | 95.99 | B |
| ATOM | 5569 | C | GLY | B | 318 | −89.068 | 49.902 | 55.706 | 1.00 | 95.14 | B |
| ATOM | 5570 | O | GLY | B | 318 | −88.708 | 50.692 | 56.579 | 1.00 | 93.87 | B |
| ATOM | 5571 | N | ASN | B | 319 | −89.313 | 48.614 | 55.936 | .00 | 95.58 | B |
| ATOM | 5572 | CA | ASN | B | 319 | −89.147 | 48.013 | 57.253 | 1.00 | 96.41 | B |
| ATOM | 5573 | CB | ASN | B | 319 | −90.170 | 46.889 | 57.471 | 1.00 | 96.95 | B |
| ATOM | 5574 | CG | ASN | B | 319 | −90.469 | 46.115 | 56.198 | 1.00 | 98.94 | B |
| ATOM | 5575 | OD1 | ASN | B | 319 | −91.247 | 46.567 | 55.355 | 1.00 | 100.00 | B |
| ATOM | 5576 | ND2 | ASN | B | 319 | −89.843 | 44.951 | 56.046 | 1.00 | 99.65 | B |
| ATOM | 5577 | C | ASN | B | 319 | −87.729 | 47.465 | 57.390 | 1.00 | 95.60 | B |
| ATOM | 5578 | O | ASN | B | 319 | −87.151 | 46.950 | 56.426 | 1.00 | 94.76 | B |
| ATOM | 5579 | N | PRO | B | 320 | −87.149 | 47.591 | 58.596 | 1.00 | 95.47 | B |
| ATOM | 5580 | CD | PRO | B | 320 | −87.791 | 48.282 | 59.730 | 1.00 | 95.16 | B |
| ATOM | 5581 | CA | PRO | B | 320 | −85.801 | 47.144 | 58.968 | 1.00 | 95.73 | B |
| ATOM | 5582 | CB | PRO | B | 320 | −85.806 | 47.290 | 60.486 | 1.00 | 94.89 | B |
| ATOM | 5583 | CG | PRO | B | 320 | −86.624 | 48.525 | 60.668 | 1.00 | 95.26 | B |
| ATOM | 5584 | C | PRO | B | 320 | −85.421 | 45.731 | 58.538 | 1.00 | 95.71 | B |
| ATOM | 5585 | O | PRO | B | 320 | −86.087 | 45.099 | 57.712 | 1.00 | 96.40 | B |
| ATOM | 5586 | N | PHE | B | 321 | −84.339 | 45.243 | 59.130 | .00 | 95.82 | B |
| ATOM | 5587 | CA | PHE | B | 321 | −83.830 | 43.915 | 58.842 | 1.00 | 95.81 | B |
| ATOM | 5588 | CB | PHE | B | 321 | −82.575 | 44.033 | 57.983 | 1.00 | 95.85 | B |
| ATOM | 5589 | CG | PHE | B | 321 | −82.191 | 42.765 | 57.300 | 1.00 | 96.09 | B |
| ATOM | 5590 | CD1 | PHE | B | 321 | −83.117 | 42.066 | 56.538 | 1.00 | 96.82 | B |
| ATOM | 5591 | CD2 | PHE | B | 321 | −80.894 | 42.278 | 57.394 | 1.00 | 96.73 | B |
| ATOM | 5592 | CE1 | PHE | B | 321 | −82.757 | 40.899 | 55.876 | .1.00 | 96.58 | B |
| ATOM | 5593 | CE2 | PHE | B | 321 | −80.521 | 41.112 | 56.736 | 1.00 | 96.76 | B |
| ATOM | 5594 | CZ | PHE | B | 321 | −81.456 | 40.422 | 55.976 | 1.00 | 96.57 | B |
| ATOM | 5595 | C | PHE | B | 321 | −83.507 | 43.258 | 60.176 | .00 | 95.68 | B |
| ATOM | 5596 | O | PHE | B | 321 | −83.311 | 42.046 | 60.259 | .00 | 95.63 | B |
| ATOM | 5597 | N | LEU | B | 322 | −83.467 | 44.083 | 61.219 | .00 | 95.61 | B |
| ATOM | 5598 | CA | LEU | B | 322 | −83.171 | 43.632 | 62.572 | .00 | 95.58 | B |
| ATOM | 5599 | CB | LEU | B | 322 | −84.366 | 42.874 | 63.158 | .00 | 95.35 | B |
| ATOM | 5600 | CG | LEU | B | 322 | −85.628 | 43.701 | 63.419 | .00 | 95.17 | B |
| ATOM | 5601 | CD1 | LEU | B | 322 | −86.282 | 44.088 | 62.101 | .00 | 95.06 | B |
| ATOM | 5602 | CD2 | LEU | B | 322 | −86.592 | 42.895 | 64.267 | .00 | 95.06 | B |
| ATOM | 5603 | C | LEU | B | 322 | −81.929 | 42.757 | 62.619 | .00 | 95.73 | B |
| ATOM | 5604 | O | LEU | B | 322 | −81.242 | 42.582 | 61.612 | .00 | 95.69 | B |
| ATOM | 5605 | N | LEU | B | 323 | −81.641 | 42.210 | 63.796 | .00 | 95.97 | B |
| ATOM | 5606 | CA | LEU | B | 323 | −80.471 | 41.363 | 63.965 | 1.00 | 96.32 | B |
| ATOM | 5607 | CB | LEU | B | 323 | −79.212 | 42.159 | 63.584 | 1.00 | 96.38 | B |
| ATOM | 5608 | CG | LEU | B | 323 | −79.144 | 43.645 | 63.963 | 1.00 | 95.99 | B |
| ATOM | 5609 | CD1 | LEU | B | 323 | −78.450 | 43.822 | 65.311 | 1.00 | 95.30 | B |
| ATOM | 5610 | CD2 | LEU | B | 323 | −78.383 | 44.400 | 62.873 | 1.00 | 95.29 | B |
| ATOM | 5611 | C | LEU | B | 323 | −80.347 | 40.779 | 65.377 | 1.00 | 96.33 | B |
| ATOM | 5612 | O | LEU | B | 323 | −79.248 | 40.548 | 65.880 | 1.00 | 96.39 | B |
| ATOM | 5613 | N | ASN | B | 324 | −81.487 | 40.522 | 66.008 | .00 | 95.87 | B |
| ATOM | 5614 | CA | ASN | B | 324 | −81.490 | 39.959 | 67.351 | .00 | 95.53 | B |
| ATOM | 5615 | CB | ASN | B | 324 | −82.881 | 40.094 | 67.976 | .00 | 95.27 | B |
| ATOM | 5616 | CG | ASN | B | 324 | −83.955 | 39.398 | 67.165 | .00 | 95.06 | B |
| ATOM | 5617 | OD1 | ASN | B | 324 | −84.178 | 39.723 | 66.000 | .00 | 94.94 | B |
| ATOM | 5618 | ND2 | ASN | B | 324 | −84.627 | 38.432 | 67.780 | .00 | 94.94 | B |
| ATOM | 5619 | C | ASN | B | 324 | −81.073 | 38.490 | 67.332 | .00 | 95.46 | B |
| ATOM | 5620 | O | ASN | B | 324 | −81.519 | 37.701 | 68.165 | .00 | 95.38 | B |
| ATOM | 5621 | N | PHE | B | 325 | −80.217 | 38.126 | 66.379 | .00 | 95.44 | B |
| ATOM | 5622 | CA | PHE | B | 325 | −79.750 | 36.748 | 66.268 | .00 | 95.51 | B |
| ATOM | 5623 | CB | PHE | B | 325 | −80.898 | 35.845 | 65.798 | .00 | 95.00 | B |
| ATOM | 5624 | CG | PHE | B | 325 | −81.516 | 36.265 | 64.491 | .00 | 94.51 | B |
| ATOM | 5625 | CD1 | PHE | B | 325 | −80.866 | 36.023 | 63.285 | .00 | 94.29 | B |
| ATOM | 5626 | CD2 | PHE | B | 325 | −82.751 | 36.905 | 64.468 | .00 | 94.29 | B |
| ATOM | 5627 | CE1 | PHE | B | 325 | −81.437 | 36.412 | 62.074 | .00 | 94.13 | B |
| ATOM | 5628 | CE2 | PHE | B | 325 | −83.331 | 37.298 | 63.263 | .00 | 94.13 | B |
| ATOM | 5629 | CZ | PHE | B | 325 | −82.672 | 37.051 | 62.064 | .00 | 94.08 | B |
| ATOM | 5630 | C | PHE | B | 325 | −78.534 | 36.568 | 65.356 | .00 | 95.87 | B |
| ATOM | 5631 | O | PHE | B | 325 | −78.352 | 35.504 | 64.764 | .00 | 95.88 | B |
| ATOM | 5632 | N | ILE | B | 326 | −77.701 | 37.601 | 65.249 | 1.00 | 96.51 | B |
| ATOM | 5633 | CA | ILE | B | 326 | −76.497 | 37.522 | 64.412 | 1.00 | 96.77 | B |
| ATOM | 5634 | CB | ILE | B | 326 | −75.808 | 38.925 | 64.228 | 1.00 | 96.87 | B |
| ATOM | 5635 | CG2 | ILE | B | 326 | −76.855 | 39.967 | 63.858 | 1.00 | 96.22 | B |
| ATOM | 5636 | CG1 | ILE | B | 326 | −75.048 | 39.346 | 65.493 | 1.00 | 95.51 | B |
| ATOM | 5637 | CD1 | ILE | B | 326 | −73.563 | 39.072 | 65.427 | 1.00 | 93.28 | B |
| ATOM | 5638 | C | ILE | B | 326 | −75.500 | 36.533 | 65.030 | 1.00 | 96.30 | B |
| ATOM | 5639 | O | ILE | B | 326 | −75.421 | 36.396 | 66.255 | 1.00 | 96.53 | B |
| ATOM | 5640 | N | PRO | B | 327 | −74.724 | 35.832 | 64.185 | 1.00 | 95.44 | B |
| ATOM | 5641 | CD | PRO | B | 327 | −74.818 | 35.855 | 62.714 | 1.00 | 94.77 | B |
| ATOM | 5642 | CA | PRO | B | 327 | −73.732 | 34.842 | 64.627 | 1.00 | 94.52 | B |
| ATOM | 5643 | CB | PRO | B | 327 | −73.195 | 34.284 | 63.307 | 1.00 | 94.83 | B |
| ATOM | 5644 | CG | PRO | B | 327 | −74.350 | 34.473 | 62.357 | 1.00 | 95.21 | B |
| ATOM | 5645 | C | PRO | B | 327 | −72.602 | 35.332 | 65.550 | 1.00 | 93.77 | B |
| ATOM | 5646 | O | PRO | B | 327 | −71.496 | 34.789 | 65.505 | 1.00 | 94.10 | B |
| ATOM | 5647 | N | GLU | B | 328 | −72.885 | 36.334 | 66.385 | 1.00 | 92.31 | B |
| ATOM | 5648 | CA | GLU | B | 328 | −71.904 | 36.902 | 67.325 | 1.00 | 90.14 | B |
| ATOM | 5649 | CB | GLU | B | 328 | −72.480 | 36.939 | 68.746 | 1.00 | 92.27 | B |
| ATOM | 5650 | CG | GLU | B | 328 | −73.651 | 37.893 | 68.962 | 1.00 | 94.43 | B |
| ATOM | 5651 | CD | GLU | B | 328 | −74.339 | 37.661 | 70.308 | 1.00 | 96.29 | B |
| ATOM | 5652 | OE1 | GLU | B | 328 | −73.626 | 37.616 | 71.342 | 1.00 | 96.01 | B |
| ATOM | 5653 | OE2 | GLU | B | 328 | −75.588 | 37.523 | 70.330 | 1.00 | 96.35 | B |
| ATOM | 5654 | C | GLU | B | 328 | −70.568 | 36.159 | 67.366 | 1.00 | 87.18 | B |
| ATOM | 5655 | O | GLU | B | 328 | −70.311 | 35.377 | 68.285 | 1.00 | 86.04 | B |
| ATOM | 5656 | N | VAL | B | 329 | −69.726 | 36.417 | 66.368 | 1.00 | 83.38 | B |
| ATOM | 5657 | CA | VAL | B | 329 | −68.411 | 35.800 | 66.263 | 1.00 | 79.67 | B |
| ATOM | 5658 | CB | VAL | B | 329 | −67.810 | 36.048 | 64.865 | 1.00 | 79.84 | B |
| ATOM | 5659 | CG1 | VAL | B | 329 | −66.432 | 35.424 | 64.769 | 1.00 | 81.26 | B |
| ATOM | 5660 | CG2 | VAL | B | 329 | −68.720 | 35.471 | 63.797 | 1.00 | 79.41 | B |
| ATOM | 5661 | C | VAL | B | 329 | −67.463 | 36.383 | 67.312 | 1.00 | 77.47 | B |
| ATOM | 5662 | O | VAL | B | 329 | −67.478 | 37.589 | 67.562 | 1.00 | 77.69 | B |
| ATOM | 5663 | N | ASP | B | 330 | −66.642 | 35.533 | 67.924 | 1.00 | 74.24 | B |
| ATOM | 5664 | CA | ASP | B | 330 | −65.689 | 35.998 | 68.933 | 1.00 | 71.17 | B |
| ATOM | 5665 | CB | ASP | B | 330 | −65.455 | 34.900 | 69.977 | 1.00 | 72.23 | B |
| ATOM | 5666 | CG | ASP | B | 330 | −66.739 | 34.472 | 70.663 | .00 | 71.59 | B |
| ATOM | 5667 | OD1 | ASP | B | 330 | −67.440 | 35.348 | 71.212 | .00 | 71.64 | B |
| ATOM | 5668 | OD2 | ASP | B | 330 | −67.049 | 33.262 | 70.652 | .00 | 71.67 | B |
| ATOM | 5669 | C | ASP | B | 330 | −64.371 | 36.411 | 68.265 | 1.00 | 68.22 | B |
| ATOM | 5670 | O | ASP | B | 330 | −64.160 | 36.138 | 67.078 | 1.00 | 67.28 | B |
| ATOM | 5671 | N | TYR | B | 331 | −63.492 | 37.074 | 69.016 | 1.00 | 64.73 | B |
| ATOM | 5672 | CA | TYR | B | 331 | −62.221 | 37.535 | 68.459 | 1.00 | 60.60 | B |
| ATOM | 5673 | CB | TYR | B | 331 | −62.288 | 39.027 | 68.152 | 1.00 | 57.76 | B |
| ATOM | 5674 | CG | TYR | B | 331 | −63.275 | 39.361 | 67.076 | 1.00 | 58.23 | B |
| ATOM | 5675 | CD1 | TYR | B | 331 | −64.629 | 39.472 | 67.366 | 1.00 | 58.41 | B |
| ATOM | 5676 | CE1 | TYR | B | 331 | −65.558 | 39.692 | 66.363 | 1.00 | 60.36 | B |
| ATOM | 5677 | CD2 | TYR | B | 331 | −62.867 | 39.488 | 65.747 | 1.00 | 58.01 | B |
| ATOM | 5678 | CE2 | TYR | B | 331 | −63.781 | 39.704 | 64.733 | 1.00 | 59.46 | B |
| ATOM | 5679 | CZ | TYR | B | 331 | −65.133 | 39.803 | 65.044 | 1.00 | 61.61 | B |
| ATOM | 5680 | OH | TYR | B | 331 | −66.066 | 39.978 | 64.038 | 1.00 | 63.14 | B |
| ATOM | 5681 | C | TYR | B | 331 | −61.013 | 37.298 | 69.329 | 1.00 | 59.37 | B |
| ATOM | 5682 | O | TYR | B | 331 | −61.115 | 37.252 | 70.551 | 1.00 | 60.47 | B |
| ATOM | 5683 | N | VAL | B | 332 | −59.858 | 37.160 | 68.691 | 1.00 | 57.81 | B |
| ATOM | 5684 | CA | VAL | B | 332 | −58.624 | 36.969 | 69.428 | 1.00 | 55.70 | B |
| ATOM | 5685 | CB | VAL | B | 332 | −57.589 | 36.175 | 68.640 | 1.00 | 56.34 | B |
| ATOM | 5686 | CG1 | VAL | B | 332 | −56.322 | 36.073 | 69.461 | 1.00 | 57.69 | B |
| ATOM | 5687 | CG2 | VAL | B | 332 | −58.115 | 34.795 | 68.306 | 1.00 | 56.95 | B |
| ATOM | 5688 | C | VAL | B | 332 | −58.033 | 38.342 | 69.674 | 1.00 | 54.85 | B |
| ATOM | 5689 | O | VAL | B | 332 | −57.637 | 39.035 | 68.732 | 1.00 | 54.73 | B |
| ATOM | 5690 | N | PRO | B | 333 | −57.988 | 38.766 | 70.944 | 1.00 | 54.10 | B |
| ATOM | 5691 | CD | PRO | B | 333 | −58.631 | 38.087 | 72.086 | 1.00 | 52.35 | B |
| ATOM | 5692 | CA | PRO | B | 333 | −57.442 | 40.062 | 71.360 | 1.00 | 53.90 | B |
| ATOM | 5693 | CB | PRO | B | 333 | −58.156 | 40.308 | 72.674 | 1.00 | 53.46 | B |
| ATOM | 5694 | CG | PRO | B | 333 | −58.173 | 38.920 | 73.264 | 1.00 | 50.51 | B |
| ATOM | 5695 | C | PRO | B | 333 | −55.931 | 39.948 | 71.566 | 1.00 | 53.58 | B |
| ATOM | 5696 | O | PRO | B | 333 | −55.442 | 38.909 | 72.022 | 1.00 | 54.82 | B |
| ATOM | 5697 | N | LEU | B | 334 | −55.185 | 40.993 | 71.237 | 1.00 | 51.61 | B |
| ATOM | 5698 | CA | LEU | B | 334 | −53.742 | 40.924 | 71.443 | 1.00 | 50.78 | B |
| ATOM | 5699 | CB | LEU | B | 334 | −53.152 | 39.754 | 70.636 | 1.00 | 50.03 | B |
| ATOM | 5700 | CG | LEU | B | 334 | −52.463 | 39.946 | 69.290 | 1.00 | 49.32 | B |
| ATOM | 5701 | CD1 | LEU | B | 334 | −52.432 | 38.601 | 68.628 | 1.00 | 50.83 | B |
| ATOM | 5702 | CD2 | LEU | B | 334 | −53.195 | 40.917 | 68.408 | 1.00 | 49.78 | B |
| ATOM | 5703 | C | LEU | B | 334 | −53.039 | 42.223 | 71.092 | 1.00 | 49.27 | B |
| ATOM | 5704 | O | LEU | B | 334 | −53.535 | 43.003 | 70.281 | 1.00 | 48.97 | B |
| ATOM | 5705 | N | SER | B | 335 | −51.896 | 42.481 | 71.716 | 1.00 | 47.50 | B |
| ATOM | 5706 | CA | SER | B | 335 | −51.193 | 43.700 | 71.376 | 1.00 | 48.27 | B |
| ATOM | 5707 | CB | SER | B | 335 | −51.162 | 44.650 | 72.575 | 1.00 | 48.48 | B |
| ATOM | 5708 | OG | SER | B | 335 | −50.830 | 43.958 | 73.745 | 1.00 | 49.07 | B |
| ATOM | 5709 | C | SER | B | 335 | −49.789 | 43.447 | 70.838 | 1.00 | 47.28 | B |
| ATOM | 5710 | O | SER | B | 335 | −49.123 | 42.490 | 71.226 | 1.00 | 45.79 | B |
| ATOM | 5711 | N | VAL | B | 336 | −49.379 | 44.296 | 69.897 | 1.00 | 47.27 | B |
| ATOM | 5712 | CA | VAL | B | 336 | −48.054 | 44.227 | 69.280 | 1.00 | 46.50 | B |
| ATOM | 5713 | CB | VAL | B | 336 | −48.062 | 44.740 | 67.849 | 1.00 | 43.61 | B |
| ATOM | 5714 | CG1 | VAL | B | 336 | −46.667 | 44.743 | 67.312 | 1.00 | 41.27 | B |
| ATOM | 5715 | CG2 | VAL | B | 336 | −48.939 | 43.881 | 66.999 | 1.00 | 45.44 | B |
| ATOM | 5716 | C | VAL | B | 336 | −47.121 | 45.133 | 70.052 | 1.00 | 48.04 | B |
| ATOM | 5717 | O | VAL | B | 336 | −47.219 | 46.359 | 69.969 | 1.00 | 48.10 | B |
| ATOM | 5718 | N | VAL | B | 337 | −46.216 | 44.541 | 70.812 | 1.00 | 49.35 | B |
| ATOM | 5719 | CA | VAL | B | 337 | −45.276 | 45.343 | 71.578 | 1.00 | 50.03 | B |
| ATOM | 5720 | CB | VAL | B | 337 | −45.059 | 44.743 | 72.982 | 1.00 | 50.28 | B |
| ATOM | 5721 | CG1 | VAL | B | 337 | −44.036 | 45.571 | 73.751 | 1.00 | 51.48 | B |
| ATOM | 5722 | CG2 | VAL | B | 337 | −46.383 | 44.686 | 73.731 | 1.00 | 50.19 | B |
| ATOM | 5723 | C | VAL | B | 337 | −43.934 | 45.442 | 70.857 | 1.00 | 49.63 | B |
| ATOM | 5724 | O | VAL | B | 337 | −43.432 | 44.460 | 70.308 | 1.00 | 48.90 | B |
| ATOM | 5725 | N | ILE | B | 338 | −43.367 | 46.638 | 70.845 | 1.00 | 50.03 | B |
| ATOM | 5726 | CA | ILE | B | 338 | −42.069 | 46.851 | 70.212 | 1.00 | 51.71 | B |
| ATOM | 5727 | CB | ILE | B | 338 | −42.151 | 47.998 | 69.151 | 1.00 | 50.62 | B |
| ATOM | 5728 | CG2 | ILE | B | 338 | −40.859 | 48.756 | 69.085 | 1.00 | 51.19 | B |
| ATOM | 5729 | CG1 | ILE | B | 338 | −42.440 | 47.422 | 67.769 | 1.00 | 50.85 | B |
| ATOM | 5730 | CD1 | ILE | B | 338 | −43.673 | 46.577 | 67.717 | 1.00 | 52.34 | B |
| ATOM | 5731 | C | ILE | B | 338 | −41.001 | 47.186 | 71.265 | 1.00 | 51.66 | B |
| ATOM | 5732 | O | ILE | B | 338 | −41.227 | 48.000 | 72.153 | 1.00 | 51.75 | B |
| ATOM | 5733 | N | THR | B | 339 | −39.851 | 46.532 | 71.193 | 1.00 | 52.65 | B |
| ATOM | 5734 | CA | THR | B | 339 | −38.771 | 46.839 | 72.130 | 1.00 | 54.80 | B |
| ATOM | 5735 | CB | THR | B | 339 | −38.597 | 45.786 | 73.243 | 1.00 | 54.90 | B |
| ATOM | 5736 | OG1 | THR | B | 339 | −38.121 | 44.561 | 72.672 | 1.00 | 55.53 | B |
| ATOM | 5737 | CG2 | THR | B | 339 | −39.910 | 45.552 | 73.971 | 1.00 | 54.76 | B |
| ATOM | 5738 | C | THR | B | 339 | −37.482 | 46.889 | 71.341 | 1.00 | 55.48 | B |
| ATOM | 5739 | O | THR | B | 339 | −37.429 | 46.457 | 70.187 | 1.00 | 56.25 | B |
| ATOM | 5740 | N | THR | B | 340 | −36.433 | 47.405 | 71.959 | 1.00 | 56.03 | B |
| ATOM | 5741 | CA | THR | B | 340 | −35.177 | 47.493 | 71.248 | 1.00 | 56.59 | B |
| ATOM | 5742 | CB | THR | B | 340 | −35.131 | 48.829 | 70.462 | 1.00 | 55.15 | B |
| ATOM | 5743 | OG1 | THR | B | 340 | −34.181 | 48.736 | 69.399 | 1.00 | 55.94 | B |
| ATOM | 5744 | CG2 | THR | B | 340 | −34.764 | 49.983 | 71.367 | 1.00 | 54.51 | B |
| ATOM | 5745 | C | THR | B | 340 | −34.010 | 47.384 | 72.219 | 1.00 | 57.14 | B |
| ATOM | 5746 | O | THR | B | 340 | −33.835 | 48.261 | 73.046 | 1.00 | 57.82 | B |
| ATOM | 5747 | N | PHE | B | 341 | −33.222 | 46.308 | 72.148 | 1.00 | 57.13 | B |
| ATOM | 5748 | CA | PHE | B | 341 | −32.077 | 46.187 | 73.059 | 1.00 | 58.02 | B |
| ATOM | 5749 | CB | PHE | B | 341 | −32.018 | 44.812 | 73.720 | 1.00 | 52.87 | B |
| ATOM | 5750 | CG | PHE | B | 341 | −33.180 | 44.514 | 74.618 | 1.00 | 50.84 | B |
| ATOM | 5751 | CD1 | PHE | B | 341 | −34.470 | 44.417 | 74.107 | 1.00 | 49.97 | B |
| ATOM | 5752 | CD2 | PHE | B | 341 | −32.979 | 44.281 | 75.971 | 1.00 | 49.21 | B |
| ATOM | 5753 | CE1 | PHE | B | 341 | −35.542 | 44.087 | 74.936 | 1.00 | 48.71 | B |
| ATOM | 5754 | CE2 | PHE | B | 341 | −34.043 | 43.952 | 76.810 | 1.00 | 47.73 | B |
| ATOM | 5755 | CZ | PHE | B | 341 | −35.328 | 43.853 | 76.294 | 1.00 | 47.81 | B |
| ATOM | 5756 | C | PHE | B | 341 | −30.761 | 46.423 | 72.333 | 1.00 | 61.04 | B |
| ATOM | 5757 | O | PHE | B | 341 | −30.625 | 46.099 | 71.146 | 1.00 | 62.86 | B |
| ATOM | 5758 | N | LYS | B | 342 | −29.793 | 47.011 | 73.030 | 1.00 | 63.35 | B |
| ATOM | 5759 | CA | LYS | B | 342 | −28.491 | 47.233 | 72.416 | 1.00 | 65.06 | B |
| ATOM | 5760 | CB | LYS | B | 342 | −27.607 | 48.105 | 73.321 | 1.00 | 65.77 | B |
| ATOM | 5761 | CG | LYS | B | 342 | −27.472 | 49.562 | 72.852 | 1.00 | 66.14 | B |
| ATOM | 5762 | CD | LYS | B | 342 | −26.686 | 50.411 | 73.835 | 1.00 | 67.36 | B |
| ATOM | 5763 | CE | LYS | B | 342 | −27.404 | 50.492 | 75.176 | 1.00 | 70.63 | B |
| ATOM | 5764 | NZ | LYS | B | 342 | −26.655 | 51.261 | 76.218 | 1.00 | 72.36 | B |
| ATOM | 5765 | C | LYS | B | 342 | −27.907 | 45.835 | 72.268 | 1.00 | 65.93 | B |
| ATOM | 5766 | O | LYS | B | 342 | −27.972 | 45.032 | 73.208 | 1.00 | 64.55 | B |
| ATOM | 5767 | N | ARG | B | 343 | −27.361 | 45.534 | 71.091 | 1.00 | 68.17 | B |
| ATOM | 5768 | CA | ARG | B | 343 | −26.785 | 44.209 | 70.837 | 1.00 | 70.09 | B |
| ATOM | 5769 | CB | ARG | B | 343 | −26.056 | 44.198 | 69.502 | 1.00 | 69.06 | B |
| ATOM | 5770 | CG | ARG | B | 343 | −26.989 | 44.500 | 68.361 | 1.00 | 71.03 | B |
| ATOM | 5771 | CD | ARG | B | 343 | −26.449 | 44.054 | 67.029 | 1.00 | 73.81 | B |
| ATOM | 5772 | NE | ARG | B | 343 | −27.463 | 44.221 | 65.996 | 1.00 | 76.86 | B |
| ATOM | 5773 | CZ | ARG | B | 343 | −27.304 | 43.882 | 64.721 | 1.00 | 79.04 | B |
| ATOM | 5774 | NH1 | ARG | B | 343 | −26.155 | 43.351 | 64.317 | 1.00 | 79.49 | B |
| ATOM | 5775 | NH2 | ARG | B | 343 | −28.297 | 44.072 | 63.852 | 1.00 | 79.36 | B |
| ATOM | 5776 | C | ARG | B | 343 | −25.861 | 43.725 | 71.945 | 1.00 | 72.02 | B |
| ATOM | 5777 | O | ARG | B | 343 | −25.849 | 42.536 | 72.282 | 1.00 | 72.45 | B |
| ATOM | 5778 | N | GLU | B | 344 | −25.102 | 44.649 | 72.523 | 1.00 | 73.86 | B |
| ATOM | 5779 | CA | GLU | B | 344 | −24.200 | 44.309 | 73.607 | 1.00 | 75.75 | B |
| ATOM | 5780 | CB | GLU | B | 344 | −23.445 | 45.558 | 74.062 | 1.00 | 79.16 | B |
| ATOM | 5781 | CG | GLU | B | 344 | −22.481 | 46.119 | 73.019 | 1.00 | 83.58 | B |
| ATOM | 5782 | CD | GLU | B | 344 | −21.483 | 47.124 | 73.611 | 1.00 | 86.53 | B |
| ATOM | 5783 | OE1 | GLU | B | 344 | −21.916 | 48.209 | 74.075 | 1.00 | 86.67 | B |
| ATOM | 5784 | OE2 | GLU | B | 344 | −20.263 | 46.820 | 73.613 | 1.00 | 87.06 | B |
| ATOM | 5785 | C | GLU | B | 344 | −24.909 | 43.669 | 74.810 | 1.00 | 75.43 | B |
| ATOM | 5786 | O | GLU | B | 344 | −24.259 | 43.070 | 75.659 | 1.00 | 75.48 | B |
| ATOM | 5787 | N | ASN | B | 345 | −26.234 | 43.779 | 74.887 | 1.00 | 75.33 | B |
| ATOM | 5788 | CA | ASN | B | 345 | −26.965 | 43.201 | 76.020 | 1.00 | 74.82 | B |
| ATOM | 5789 | CB | ASN | B | 345 | −27.832 | 44.269 | 76.687 | 1.00 | 77.12 | B |
| ATOM | 5790 | CG | ASN | B | 345 | −27.008 | 45.368 | 77.326 | 1.00 | 77.88 | B |
| ATOM | 5791 | OD1 | ASN | B | 345 | −26.080 | 45.097 | 78.089 | 1.00 | 79.12 | B |
| ATOM | 5792 | ND2 | ASN | B | 345 | −27.349 | 46.619 | 77.024 | 1.00 | 77.74 | B |
| ATOM | 5793 | C | ASN | B | 345 | −27.824 | 41.969 | 75.714 | 1.00 | 73.54 | B |
| ATOM | 5794 | O | ASN | B | 345 | −28.577 | 41.485 | 76.576 | 1.00 | 72.20 | B |
| ATOM | 5795 | N | VAL | B | 346 | −27.720 | 41.471 | 74.487 | 1.00 | 71.03 | B |
| ATOM | 5796 | CA | VAL | B | 346 | −28.459 | 40.281 | 74.100 | 1.00 | 69.11 | B |
| ATOM | 5797 | CB | VAL | B | 346 | −29.085 | 40.448 | 72.689 | 1.00 | 68.72 | B |
| ATOM | 5798 | CG1 | VAL | B | 346 | −29.714 | 39.140 | 72.243 | 1.00 | 67.93 | B |
| ATOM | 5799 | CG2 | VAL | B | 346 | −30.147 | 41.550 | 72.713 | 1.00 | 65.93 | B |
| ATOM | 5800 | C | VAL | B | 346 | −27.448 | 39.131 | 74.117 | 1.00 | 67.75 | B |
| ATOM | 5801 | O | VAL | B | 346 | −26.701 | 38.926 | 73.161 | 1.00 | 68.20 | B |
| ATOM | 5802 | N | LYS | B | 347 | −27.419 | 38.396 | 75.221 | 1.00 | 65.17 | B |
| ATOM | 5803 | CA | LYS | B | 347 | −26.480 | 37.294 | 75.382 | 1.00 | 64.16 | B |
| ATOM | 5804 | CB | LYS | B | 347 | −26.601 | 36.693 | 76.791 | 1.00 | 65.41 | B |
| ATOM | 5805 | CG | LYS | B | 347 | −25.975 | 37.550 | 77.889 | 1.00 | 67.00 | B |
| ATOM | 5806 | CD | LYS | B | 347 | −26.532 | 37.209 | 79.251 | 1.00 | 68.13 | B |
| ATOM | 5807 | CE | LYS | B | 347 | −26.352 | 35.736 | 79.571 | 1.00 | 72.15 | B |
| ATOM | 5808 | NZ | LYS | B | 347 | −26.986 | 35.343 | 80.876 | 1.00 | 73.70 | B |
| ATOM | 5809 | C | LYS | B | 347 | −26.582 | 36.181 | 74.357 | 1.00 | 63.01 | B |
| ATOM | 5810 | O | LYS | B | 347 | −25.618 | 35.894 | 73.671 | 1.00 | 63.53 | B |
| ATOM | 5811 | N | TYR | B | 348 | −27.750 | 35.560 | 74.241 | 1.00 | 62.84 | B |
| ATOM | 5812 | CA | TYR | B | 348 | −27.932 | 34.431 | 73.325 | 1.00 | 61.21 | B |
| ATOM | 5813 | CB | TYR | B | 348 | −28.498 | 33.223 | 74.099 | 1.00 | 61.52 | B |
| ATOM | 5814 | CG | TYR | B | 348 | −27.807 | 32.971 | 75.422 | 1.00 | 62.88 | B |
| ATOM | 5815 | CD1 | TYR | B | 348 | −28.549 | 32.795 | 76.596 | 1.00 | 63.92 | B |
| ATOM | 5816 | CE1 | TYR | B | 348 | −27.926 | 32.661 | 77.842 | 1.00 | 64.80 | B |
| ATOM | 5817 | CD2 | TYR | B | 348 | −26.416 | 32.992 | 75.519 | 1.00 | 63.65 | B |
| ATOM | 5818 | CE2 | TYR | B | 348 | −25.779 | 32.859 | 76.756 | 1.00 | 65.86 | B |
| ATOM | 5819 | CZ | TYR | B | 348 | −26.541 | 32.698 | 77.918 | 1.00 | 66.59 | B |
| ATOM | 5820 | OH | TYR | B | 348 | −25.920 | 32.598 | 79.154 | 1.00 | 67.79 | B |
| ATOM | 5821 | C | TYR | B | 348 | −28.814 | 34.693 | 72.106 | 1.00 | 59.09 | B |
| ATOM | 5822 | O | TYR | B | 348 | −29.916 | 34.149 | 71.998 | 1.00 | 59.89 | B |
| ATOM | 5823 | N | PRO | B | 349 | −28.339 | 35.515 | 71.167 | 1.00 | 56.83 | B |
| ATOM | 5824 | CD | PRO | B | 349 | −27.080 | 36.266 | 71.192 | 1.00 | 55.82 | B |
| ATOM | 5825 | CA | PRO | B | 349 | −29.108 | 35.823 | 69.955 | 1.00 | 56.99 | B |
| ATOM | 5826 | CB | PRO | B | 349 | −28.209 | 36.802 | 69.208 | 1.00 | 55.48 | B |
| ATOM | 5827 | CG | PRO | B | 349 | −27.389 | 37.405 | 70.273 | 1.00 | 56.66 | B |
| ATOM | 5828 | C | PRO | B | 349 | −29.333 | 34.558 | 69.123 | 1.00 | 57.90 | B |
| ATOM | 5829 | O | PRO | B | 349 | −28.539 | 33.613 | 69.178 | 1.00 | 59.19 | B |
| ATOM | 5830 | N | LEU | B | 350 | −30.411 | 34.541 | 68.350 | 1.00 | 57.57 | B |
| ATOM | 5831 | CA | LEU | B | 350 | −30.704 | 33.402 | 67.491 | 1.00 | 56.59 | B |
| ATOM | 5832 | CB | LEU | B | 350 | −31.988 | 32.693 | 67.931 | 1.00 | 56.13 | B |
| ATOM | 5833 | CG | LEU | B | 350 | −31.884 | 31.819 | 69.184 | 1.00 | 57.07 | B |
| ATOM | 5834 | CD1 | LEU | B | 350 | −33.264 | 31.407 | 69.641 | 1.00 | 56.47 | B |
| ATOM | 5835 | CD2 | LEU | B | 350 | −31.030 | 30.600 | 68.885 | 1.00 | 56.95 | B |
| ATOM | 5836 | C | LEU | B | 350 | −30.853 | 33.935 | 66.084 | 1.00 | 56.74 | B |
| ATOM | 5837 | O | LEU | B | 350 | −31.323 | 35.052 | 65.887 | 1.00 | 57.50 | B |
| ATOM | 5838 | N | GLU | B | 351 | −30.422 | 33.151 | 65.108 | 1.00 | 57.04 | B |
| ATOM | 5839 | CA | GLU | B | 351 | −30.520 | 33.563 | 63.724 | 1.00 | 57.16 | B |
| ATOM | 5840 | CB | GLU | B | 351 | −29.341 | 33.015 | 62.929 | 1.00 | 57.54 | B |
| ATOM | 5841 | CG | GLU | B | 351 | −29.252 | 33.548 | 61.507 | 1.00 | 59.33 | B |
| ATOM | 5842 | CD | GLU | B | 351 | −29.056 | 35.051 | 61.456 | .00 | 58.47 | B |
| ATOM | 5843 | OE1 | GLU | B | 351 | −28.050 | 35.542 | 62.009 | .00 | 58.59 | B |
| ATOM | 5844 | OE2 | GLU | B | 351 | −29.909 | 35.741 | 60.860 | .00 | 58.59 | B |
| ATOM | 5845 | C | GLU | B | 351 | −31.823 | 33.013 | 63.176 | 1.00 | 57.94 | B |
| ATOM | 5846 | O | GLU | B | 351 | −32.170 | 31.860 | 63.424 | 1.00 | 58.18 | B |
| ATOM | 5847 | N | GLY | B | 352 | −32.556 | 33.842 | 62.442 | 1.00 | 58.26 | B |
| ATOM | 5848 | CA | GLY | B | 352 | −33.815 | 33.390 | 61.884 | 1.00 | 56.70 | B |
| ATOM | 5849 | C | GLY | B | 352 | −34.879 | 34.463 | 61.858 | 1.00 | 55.68 | B |
| ATOM | 5850 | O | GLY | B | 352 | −34.568 | 35.653 | 61.929 | 1.00 | 55.50 | B |
| ATOM | 5851 | N | PHE | B | 353 | −36.138 | 34.040 | 61.753 | 1.00 | 54.72 | B |
| ATOM | 5852 | CA | PHE | B | 353 | −37.257 | 34.977 | 61.701 | 1.00 | 52.52 | B |
| ATOM | 5853 | CB | PHE | B | 353 | −38.354 | 34.458 | 60.773 | 1.00 | 51.34 | B |
| ATOM | 5854 | CG | PHE | B | 353 | −37.992 | 34.516 | 59.331 | 1.00 | 51.52 | B |
| ATOM | 5855 | CD1 | PHE | B | 353 | −36.659 | 34.612 | 58.935 | 1.00 | 53.23 | B |
| ATOM | 5856 | CD2 | PHE | B | 353 | −38.975 | 34.428 | 58.355 | 1.00 | 53.88 | B |
| ATOM | 5857 | CE1 | PHE | B | 353 | −36.304 | 34.616 | 57.583 | 1.00 | 54.58 | B |
| ATOM | 5858 | CE2 | PHE | B | 353 | −38.644 | 34.427 | 57.001 | 1.00 | 54.20 | B |
| ATOM | 5859 | CZ | PHE | B | 353 | −37.301 | 34.521 | 56.613 | 1.00 | 55.97 | B |
| ATOM | 5860 | C | PHE | B | 353 | −37.837 | 35.247 | 63.067 | 1.00 | 50.63 | B |
| ATOM | 5861 | O | PHE | B | 353 | −38.256 | 36.367 | 63.340 | 1.00 | 48.85 | B |
| ATOM | 5862 | N | GLY | B | 354 | −37.865 | 34.232 | 63.926 | 1.00 | 48.63 | B |
| ATOM | 5863 | CA | GLY | B | 354 | −38.399 | 34.466 | 65.250 | 1.00 | 49.69 | B |
| ATOM | 5864 | C | GLY | B | 354 | −38.547 | 33.259 | 66.138 | 1.00 | 50.17 | B |
| ATOM | 5865 | O | GLY | B | 354 | −37.875 | 32.245 | 65.952 | 1.00 | 51.10 | B |
| ATOM | 5866 | N | VAL | B | 355 | −39.430 | 33.386 | 67.122 | 1.00 | 50.17 | B |
| ATOM | 5867 | CA | VAL | B | 355 | −39.696 | 32.317 | 68.071 | 1.00 | 51.49 | B |
| ATOM | 5868 | CB | VAL | B | 355 | −38.977 | 32.540 | 69.415 | 1.00 | 51.41 | B |
| ATOM | 5869 | CG1 | VAL | B | 355 | −39.286 | 31.393 | 70.364 | 1.00 | 51.97 | B |
| ATOM | 5870 | CG2 | VAL | B | 355 | −37.502 | 32.657 | 69.196 | 1.00 | 53.28 | B |
| ATOM | 5871 | C | VAL | B | 355 | −41.188 | 32.306 | 68.352 | 1.00 | 52.18 | B |
| ATOM | 5872 | O | VAL | B | 355 | −41.812 | 33.364 | 68.456 | 1.00 | 52.31 | B |
| ATOM | 5873 | N | LEU | B | 356 | −41.741 | 31.102 | 68.478 | 1.00 | 50.94 | B |
| ATOM | 5874 | CA | LEU | B | 356 | −43.153 | 30.897 | 68.755 | 1.00 | 49.49 | B |
| ATOM | 5875 | CB | LEU | B | 356 | −43.763 | 29.960 | 67.717 | 1.00 | 49.08 | B |
| ATOM | 5876 | CG | LEU | B | 356 | −43.815 | 30.539 | 66.315 | 1.00 | 48.75 | B |
| ATOM | 5877 | CD1 | LEU | B | 356 | −44.203 | 29.476 | 65.345 | 1.00 | 52.52 | B |
| ATOM | 5878 | CD2 | LEU | B | 356 | −44.824 | 31.643 | 66.279 | 1.00 | 50.97 | B |
| ATOM | 5879 | C | LEU | B | 356 | −43.248 | 30.268 | 70.126 | 1.00 | 49.36 | B |
| ATOM | 5880 | O | LEU | B | 356 | −42.459 | 29.403 | 70.471 | 1.00 | 50.24 | B |
| ATOM | 5881 | N | VAL | B | 357 | −44.225 | 30.686 | 70.909 | 1.00 | 49.63 | B |
| ATOM | 5882 | CA | VAL | B | 357 | −44.350 | 30.149 | 72.243 | 1.00 | 49.72 | B |
| ATOM | 5883 | CB | VAL | B | 357 | −44.387 | 31.279 | 73.286 | 1.00 | 49.73 | B |
| ATOM | 5884 | CG1 | VAL | B | 357 | −44.442 | 30.695 | 74.684 | 1.00 | 49.92 | B |
| ATOM | 5885 | CG2 | VAL | B | 357 | −43.158 | 32.173 | 73.127 | 1.00 | 48.82 | B |
| ATOM | 5886 | C | VAL | B | 357 | −45.572 | 29.274 | 72.394 | 1.00 | 50.38 | B |
| ATOM | 5887 | O | VAL | B | 357 | −46.692 | 29.754 | 72.480 | 1.00 | 49.63 | B |
| ATOM | 5888 | N | PRO | B | 358 | −45.360 | 27.957 | 72.415 | 1.00 | 52.04 | B |
| ATOM | 5889 | CD | PRO | B | 358 | −44.083 | 27.235 | 72.251 | 1.00 | 51.70 | B |
| ATOM | 5890 | CA | PRO | B | 358 | −46.477 | 27.029 | 72.564 | 1.00 | 53.14 | B |
| ATOM | 5891 | CB | PRO | B | 358 | −45.783 | 25.670 | 72.632 | 1.00 | 52.56 | B |
| ATOM | 5892 | CG | PRO | B | 358 | −44.540 | 25.877 | 71.814 | 1.00 | 50.36 | B |
| ATOM | 5893 | C | PRO | B | 358 | −47.202 | 27.367 | 73.855 | 1.00 | 54.66 | B |
| ATOM | 5894 | O | PRO | B | 358 | −46.564 | 27.578 | 74.890 | 1.00 | 54.47 | B |
| ATOM | 5895 | N | SER | B | 359 | −48.529 | 27.416 | 73.789 | 1.00 | 56.33 | B |
| ATOM | 5896 | CA | SER | B | 359 | −49.355 | 27.731 | 74.946 | 1.00 | 57.82 | B |
| ATOM | 5897 | CB | SER | B | 359 | −50.813 | 27.476 | 74.615 | 1.00 | 58.26 | B |
| ATOM | 5898 | OG | SER | B | 359 | −50.969 | 26.168 | 74.112 | 1.00 | 59.37 | B |
| ATOM | 5899 | C | SER | B | 359 | −48.973 | 26.921 | 76.173 | 1.00 | 59.47 | B |
| ATOM | 5900 | O | SER | B | 359 | −49.158 | 27.368 | 77.299 | 1.00 | 61.21 | B |
| ATOM | 5901 | N | LYS | B | 360 | −48.441 | 25.725 | 75.954 | 1.00 | 61.22 | B |
| ATOM | 5902 | CA | LYS | B | 360 | −48.031 | 24.855 | 77.054 | 1.00 | 62.43 | B |
| ATOM | 5903 | CB | LYS | B | 360 | −47.740 | 23.442 | 76.522 | 1.00 | 65.04 | B |
| ATOM | 5904 | CG | LYS | B | 360 | −47.367 | 22.431 | 77.594 | 1.00 | 66.84 | B |
| ATOM | 5905 | CD | LYS | B | 360 | −47.582 | 21.005 | 77.126 | 1.00 | 69.28 | B |
| ATOM | 5906 | CE | LYS | B | 360 | −47.425 | 20.027 | 78.297 | 1.00 | 73.80 | B |
| ATOM | 5907 | NZ | LYS | B | 360 | −47.724 | 18.595 | 77.946 | 1.00 | 75.30 | B |
| ATOM | 5908 | C | LYS | B | 360 | −46.796 | 25.402 | 77.768 | 1.00 | 61.87 | B |
| ATOM | 5909 | O | LYS | B | 360 | −46.447 | 24.969 | 78.864 | 1.00 | 61.70 | B |
| ATOM | 5910 | N | GLU | B | 361 | −46.131 | 26.361 | 77.148 | 1.00 | 61.41 | B |
| ATOM | 5911 | CA | GLU | B | 361 | −44.941 | 26.925 | 77.758 | 1.00 | 62.08 | B |
| ATOM | 5912 | CB | GLU | B | 361 | −44.042 | 27.505 | 76.665 | 1.00 | 63.75 | B |
| ATOM | 5913 | CG | GLU | B | 361 | −43.341 | 26.433 | 75.852 | 1.00 | 65.34 | B |
| ATOM | 5914 | CD | GLU | B | 361 | −42.393 | 25.592 | 76.701 | 1.00 | 68.42 | B |
| ATOM | 5915 | OE1 | GLU | B | 361 | −41.436 | 26.174 | 77.266 | 1.00 | 67.16 | B |
| ATOM | 5916 | OE2 | GLU | B | 361 | −42.604 | 24.355 | 76.805 | 1.00 | 70.46 | B |
| ATOM | 5917 | C | GLU | B | 361 | −45.225 | 27.968 | 78.846 | 1.00 | 60.95 | B |
| ATOM | 5918 | O | GLU | B | 361 | −44.307 | 28.561 | 79.411 | 1.00 | 59.78 | B |
| ATOM | 5919 | N | GLN | B | 362 | −46.496 | 28.194 | 79.143 | 1.00 | 59.87 | B |
| ATOM | 5920 | CA | GLN | B | 362 | −46.836 | 29.148 | 80.183 | 1.00 | 60.04 | B |
| ATOM | 5921 | CB | GLN | B | 362 | −48.339 | 29.431 | 80.183 | 1.00 | 57.82 | B |
| ATOM | 5922 | CG | GLN | B | 362 | −48.782 | 30.255 | 79.000 | 1.00 | 54.89 | B |
| ATOM | 5923 | CD | GLN | B | 362 | −50.266 | 30.478 | 78.976 | 1.00 | 54.52 | B |
| ATOM | 5924 | OE1 | GLN | B | 362 | −51.042 | 29.559 | 78.723 | 1.00 | 52.55 | B |
| ATOM | 5925 | NE2 | GLN | B | 362 | −50.678 | 31.711 | 79.249 | 1.00 | 57.45 | B |
| ATOM | 5926 | C | GLN | B | 362 | −46.403 | 28.552 | 81.517 | 1.00 | 61.34 | B |
| ATOM | 5927 | O | GLN | B | 362 | −45.989 | 29.261 | 82.430 | 1.00 | 61.98 | B |
| ATOM | 5928 | N | GLN | B | 363 | −46.485 | 27.236 | 81.627 | 1.00 | 63.48 | B |
| ATOM | 5929 | CA | GLN | B | 363 | −46.076 | 26.575 | 82.854 | 1.00 | 64.37 | B |
| ATOM | 5930 | CB | GLN | B | 363 | −46.611 | 25.141 | 82.876 | 1.00 | 67.32 | B |
| ATOM | 5931 | CG | GLN | B | 363 | −46.420 | 24.384 | 81.564 | 1.00 | 73.06 | B |
| ATOM | 5932 | CD | GLN | B | 363 | −47.035 | 22.985 | 81.582 | 1.00 | 76.37 | B |
| ATOM | 5933 | OE1 | GLN | B | 363 | −48.225 | 22.814 | 81.895 | 1.00 | 76.40 | B |
| ATOM | 5934 | NE2 | GLN | B | 363 | −46.226 | 21.976 | 81.240 | 1.00 | 77.34 | B |
| ATOM | 5935 | C | GLN | B | 363 | −44.552 | 26.590 | 82.979 | 1.00 | 63.30 | B |
| ATOM | 5936 | O | GLN | B | 363 | −43.986 | 25.851 | 83.774 | 1.00 | 62.22 | B |
| ATOM | 5937 | N | HIS | B | 364 | −43.899 | 27.442 | 82.187 | 1.00 | 63.19 | B |
| ATOM | 5938 | CA | HIS | B | 364 | −42.441 | 27.575 | 82.200 | 1.00 | 63.36 | B |
| ATOM | 5939 | CB | HIS | B | 364 | −41.818 | 26.913 | 80.971 | 1.00 | 66.82 | B |
| ATOM | 5940 | CG | HIS | B | 364 | −41.571 | 25.448 | 81.133 | 1.00 | 70.21 | B |
| ATOM | 5941 | CD2 | HIS | B | 364 | −42.154 | 24.374 | 80.548 | 1.00 | 72.43 | B |
| ATOM | 5942 | ND1 | HIS | B | 364 | −40.630 | 24.945 | 82.006 | 1.00 | 72.47 | B |
| ATOM | 5943 | CE1 | HIS | B | 364 | −40.643 | 23.623 | 81.953 | 1.00 | 73.39 | B |
| ATOM | 5944 | NE2 | HIS | B | 364 | −41.560 | 23.251 | 81.077 | 1.00 | 73.62 | B |
| ATOM | 5945 | C | HIS | B | 364 | −41.989 | 29.031 | 82.262 | 1.00 | 62.44 | B |
| ATOM | 5946 | O | HIS | B | 364 | −40.811 | 29.335 | 82.073 | 1.00 | 62.65 | B |
| ATOM | 5947 | N | GLY | B | 365 | −42.926 | 29.938 | 82.508 | 1.00 | 60.86 | B |
| ATOM | 5948 | CA | GLY | B | 365 | −42.556 | 31.334 | 82.627 | 1.00 | 58.65 | B |
| ATOM | 5949 | C | GLY | B | 365 | −42.703 | 32.200 | 81.401 | 1.00 | 57.76 | B |
| ATOM | 5950 | O | GLY | B | 365 | −42.574 | 33.425 | 81.484 | 1.00 | 57.87 | B |
| ATOM | 5951 | N | LEU | B | 366 | −42.949 | 31.587 | 80.254 | 1.00 | 57.03 | B |
| ATOM | 5952 | CA | LEU | B | 366 | −43.126 | 32.372 | 79.042 | 1.00 | 55.64 | B |
| ATOM | 5953 | CB | LEU | B | 366 | −42.707 | 31.553 | 77.824 | 1.00 | 57.12 | B |
| ATOM | 5954 | CG | LEU | B | 366 | −41.231 | 31.172 | 77.902 | 1.00 | 57.26 | B |
| ATOM | 5955 | CD1 | LEU | B | 366 | −40.874 | 30.242 | 76.759 | 1.00 | 57.74 | B |
| ATOM | 5956 | CD2 | LEU | B | 366 | −40.386 | 32.434 | 77.856 | 1.00 | 57.50 | B |
| ATOM | 5957 | C | LEU | B | 366 | −44.595 | 32.777 | 78.957 | 1.00 | 53.97 | B |
| ATOM | 5958 | O | LEU | B | 366 | −45.493 | 31.919 | 78.858 | 1.00 | 51.34 | B |
| ATOM | 5959 | N | ALA | B | 367 | −44.830 | 34.088 | 79.017 | 1.00 | 50.76 | B |
| ATOM | 5960 | CA | ALA | B | 367 | −46.186 | 34.613 | 78.974 | 1.00 | 49.14 | B |
| ATOM | 5961 | CB | ALA | B | 367 | −46.360 | 35.647 | 80.086 | 1.00 | 47.72 | B |
| ATOM | 5962 | C | ALA | B | 367 | −46.622 | 35.191 | 77.618 | 1.00 | 47.92 | B |
| ATOM | 5963 | O | ALA | B | 367 | −47.808 | 35.466 | 77.422 | 1.00 | 48.53 | B |
| ATOM | 5964 | N | THR | B | 368 | −45.679 | 35.355 | 76.690 | 1.00 | 45.99 | B |
| ATOM | 5965 | CA | THR | B | 368 | −45.978 | 35.894 | 75.366 | 1.00 | 46.38 | B |
| ATOM | 5966 | CB | THR | B | 368 | −44.772 | 36.689 | 74.802 | 1.00 | 48.72 | B |
| ATOM | 5967 | OG1 | THR | B | 368 | −43.719 | 35.793 | 74.400 | 1.00 | 49.95 | B |
| ATOM | 5968 | CG2 | THR | B | 368 | −44.240 | 37.637 | 75.859 | 1.00 | 49.21 | B |
| ATOM | 5969 | C | THR | B | 368 | −46.367 | 34.805 | 74.357 | 1.00 | 46.08 | B |
| ATOM | 5970 | O | THR | B | 368 | −46.334 | 33.619 | 74.679 | 1.00 | 48.25 | B |
| ATOM | 5971 | N | LEU | B | 369 | −46.738 | 35.217 | 73.143 | 1.00 | 45.24 | B |
| ATOM | 5972 | CA | LEU | B | 369 | −47.156 | 34.304 | 72.073 | 1.00 | 44.34 | B |
| ATOM | 5973 | CB | LEU | B | 369 | −48.298 | 34.916 | 71.267 | 1.00 | 43.10 | B |
| ATOM | 5974 | CG | LEU | B | 369 | −49.606 | 35.185 | 71.997 | 1.00 | 45.45 | B |
| ATOM | 5975 | CD1 | LEU | B | 369 | −50.574 | 35.941 | 71.085 | 1.00 | 45.83 | B |
| ATOM | 5976 | CD2 | LEU | B | 369 | −50.200 | 33.848 | 72.449 | 1.00 | 45.87 | B |
| ATOM | 5977 | C | LEU | B | 369 | −46.025 | 34.009 | 71.106 | 1.00 | 45.46 | B |
| ATOM | 5978 | O | LEU | B | 369 | −45.978 | 32.952 | 70.488 | 1.00 | 46.75 | B |
| ATOM | 5979 | N | GLY | B | 370 | −45.126 | 34.966 | 70.952 | 1.00 | 45.53 | B |
| ATOM | 5980 | CA | GLY | B | 370 | −44.012 | 34.787 | 70.049 | 1.00 | 45.25 | B |
| ATOM | 5981 | C | GLY | B | 370 | −43.457 | 36.155 | 69.725 | 1.00 | 46.37 | B |
| ATOM | 5982 | O | GLY | B | 370 | −44.120 | 37.165 | 69.967 | 1.00 | 46.82 | B |
| ATOM | 5983 | N | THR | B | 371 | −42.244 | 36.205 | 69.187 | 1.00 | 45.71 | B |
| ATOM | 5984 | CA | THR | B | 371 | −41.650 | 37.480 | 68.853 | 1.00 | 45.32 | B |
| ATOM | 5985 | CB | THR | B | 371 | −40.791 | 38.047 | 70.054 | 1.00 | 44.74 | B |
| ATOM | 5986 | OG1 | THR | B | 371 | −39.409 | 38.133 | 69.676 | 1.00 | 43.22 | B |
| ATOM | 5987 | CG2 | THR | B | 371 | −40.956 | 37.179 | 71.318 | 1.00 | 38.98 | B |
| ATOM | 5988 | C | THR | B | 371 | −40.816 | 37.365 | 67.580 | 1.00 | 46.77 | B |
| ATOM | 5989 | O | THR | B | 371 | −40.115 | 36.374 | 67.360 | 1.00 | 47.72 | B |
| ATOM | 5990 | N | LEU | B | 372 | −40.926 | 38.380 | 66.733 | 1.00 | 46.65 | B |
| ATOM | 5991 | CA | LEU | B | 372 | −40.204 | 38.429 | 65.480 | 1.00 | 47.13 | B |
| ATOM | 5992 | CB | LEU | B | 372 | −41.010 | 39.219 | 64.463 | 1.00 | 44.56 | B |
| ATOM | 5993 | CG | LEU | B | 372 | −42.289 | 38.553 | 63.972 | 1.00 | 46.19 | B |
| ATOM | 5994 | CD1 | LEU | B | 372 | −43.046 | 39.524 | 63.082 | 1.00 | 45.99 | B |
| ATOM | 5995 | CD2 | LEU | B | 372 | −41.955 | 37.274 | 63.209 | 1.00 | 45.91 | B |
| ATOM | 5996 | C | LEU | B | 372 | −38.840 | 39.085 | 65.649 | 1.00 | 49.42 | B |
| ATOM | 5997 | O | LEU | B | 372 | −38.662 | 39.938 | 66.512 | 1.00 | 51.40 | B |
| ATOM | 5998 | N | PHE | B | 373 | −37.879 | 38.679 | 64.825 | 1.00 | 50.12 | B |
| ATOM | 5999 | CA | PHE | B | 373 | −36.541 | 39.247 | 64.864 | 1.00 | 49.14 | B |
| ATOM | 6000 | CB | PHE | B | 373 | −35.491 | 38.156 | 64.645 | 1.00 | 47.78 | B |
| ATOM | 6001 | CG | PHE | B | 373 | −35.502 | 37.083 | 65.701 | 1.00 | 48.36 | B |
| ATOM | 6002 | CD1 | PHE | B | 373 | −34.727 | 35.942 | 65.556 | 1.00 | 49.98 | B |
| ATOM | 6003 | CD2 | PHE | B | 373 | −36.300 | 37.200 | 66.836 | 1.00 | 50.25 | B |
| ATOM | 6004 | CE1 | PHE | B | 373 | −34.748 | 34.928 | 66.523 | 1.00 | 50.48 | B |
| ATOM | 6005 | CE2 | PHE | B | 373 | −36.327 | 36.187 | 67.816 | 1.00 | 50.76 | B |
| ATOM | 6006 | CZ | PHE | B | 373 | −35.550 | 35.054 | 67.654 | 1.00 | 50.68 | B |
| ATOM | 6007 | C | PHE | B | 373 | −36.525 | 40.277 | 63.748 | 1.00 | 49.32 | B |
| ATOM | 6008 | O | PHE | B | 373 | −35.908 | 40.098 | 62.707 | 1.00 | 47.95 | B |
| ATOM | 6009 | N | SER | B | 374 | −37.231 | 41.367 | 64.011 | 1.00 | 51.38 | B |
| ATOM | 6010 | CA | SER | B | 374 | −37.409 | 42.486 | 63.094 | 1.00 | 53.93 | B |
| ATOM | 6011 | CB | SER | B | 374 | −38.179 | 43.579 | 63.818 | 1.00 | 54.20 | B |
| ATOM | 6012 | OG | SER | B | 374 | −39.268 | 43.009 | 64.530 | 1.00 | 54.64 | B |
| ATOM | 6013 | C | SER | B | 374 | −36.195 | 43.099 | 62.411 | 1.00 | 55.88 | B |
| ATOM | 6014 | O | SER | B | 374 | −36.171 | 43.203 | 61.182 | 1.00 | 55.33 | B |
| ATOM | 6015 | N | SER | B | 375 | −35.201 | 43.531 | 63.186 | 1.00 | 60.01 | B |
| ATOM | 6016 | CA | SER | B | 375 | −34.005 | 44.144 | 62.592 | 1.00 | 62.59 | B |
| ATOM | 6017 | CB | SER | B | 375 | −33.200 | 44.933 | 63.641 | 1.00 | 60.92 | B |
| ATOM | 6018 | OG | SER | B | 375 | −32.891 | 44.142 | 64.770 | 1.00 | 61.32 | B |
| ATOM | 6019 | C | SER | B | 375 | −33.127 | 43.099 | 61.908 | 1.00 | 63.98 | B |
| ATOM | 6020 | O | SER | B | 375 | −32.326 | 43.432 | 61.032 | 1.00 | 63.71 | B |
| ATOM | 6021 | N | MSE | B | 376 | −33.287 | 41.834 | 62.293 | 1.00 | 66.11 | B |
| ATOM | 6022 | CA | MSE | B | 376 | −32.512 | 40.773 | 61.664 | 1.00 | 68.97 | B |
| ATOM | 6023 | CB | MSE | B | 376 | −32.520 | 39.511 | 62.516 | 1.00 | 71.08 | B |
| ATOM | 6024 | CG | MSE | B | 376 | −31.290 | 39.390 | 63.374 | 1.00 | 76.01 | B |
| ATOM | 6025 | SE | MSE | B | 376 | −31.115 | 37.631 | 64.095 | 1.00 | 87.55 | B |
| ATOM | 6026 | CE | MSE | B | 376 | −31.533 | 38.022 | 65.981 | 1.00 | 82.89 | B |
| ATOM | 6027 | C | MSE | B | 376 | −33.051 | 40.467 | 60.275 | 1.00 | 69.03 | B |
| ATOM | 6028 | O | MSE | B | 376 | −32.292 | 40.256 | 59.329 | 1.00 | 68.28 | B |
| ATOM | 6029 | N | MSE | B | 377 | −34.370 | 40.453 | 60.156 | 1.00 | 70.30 | B |
| ATOM | 6030 | CA | MSE | B | 377 | −35.009 | 40.191 | 58.879 | 1.00 | 71.41 | B |
| ATOM | 6031 | CB | MSE | B | 377 | −36.496 | 39.950 | 59.077 | 1.00 | 73.16 | B |
| ATOM | 6032 | CG | MSE | B | 377 | −36.832 | 38.793 | 59.955 | 1.00 | 76.98 | B |
| ATOM | 6033 | SE | MSE | B | 377 | −38.736 | 38.593 | 59.917 | 1.00 | 85.38 | B |
| ATOM | 6034 | CE | MSE | B | 377 | −39.098 | 38.800 | 61.795 | 1.00 | 82.12 | B |
| ATOM | 6035 | C | MSE | B | 377 | −34.838 | 41.410 | 57.987 | 1.00 | 71.35 | B |
| ATOM | 6036 | O | MSE | B | 377 | −34.589 | 41.296 | 56.785 | 1.00 | 71.23 | B |
| ATOM | 6037 | N | PHE | B | 378 | −34.995 | 42.578 | 58.597 | 1.00 | 70.54 | B |
| ATOM | 6038 | CA | PHE | B | 378 | −34.890 | 43.839 | 57.896 | 1.00 | 70.02 | B |
| ATOM | 6039 | CB | PHE | B | 378 | −36.265 | 44.494 | 57.813 | 1.00 | 68.70 | B |
| ATOM | 6040 | CG | PHE | B | 378 | −37.354 | 43.574 | 57.351 | 1.00 | 68.30 | B |
| ATOM | 6041 | CD1 | PHE | B | 378 | −37.489 | 43.251 | 56.002 | 1.00 | 67.81 | B |
| ATOM | 6042 | CD2 | PHE | B | 378 | −38.249 | 43.021 | 58.270 | 1.00 | 67.64 | B |
| ATOM | 6043 | CE1 | PHE | B | 378 | −38.502 | 42.388 | 55.576 | 1.00 | 68.09 | B |
| ATOM | 6044 | CE2 | PHE | B | 378 | −39.266 | 42.156 | 57.858 | 1.00 | 66.01 | B |
| ATOM | 6045 | CZ | PHE | B | 378 | −39.394 | 41.839 | 56.513 | 1.00 | 67.34 | B |
| ATOM | 6046 | C | PHE | B | 378 | −33.959 | 44.737 | 58.698 | 1.00 | 70.99 | B |
| ATOM | 6047 | O | PHE | B | 378 | −34.414 | 45.530 | 59.518 | 1.00 | 72.39 | B |
| ATOM | 6048 | N | PRO | B | 379 | −32.641 | 44.612 | 58.492 | 1.00 | 70.54 | B |
| ATOM | 6049 | CD | PRO | B | 379 | −31.929 | 43.618 | 57.676 | 1.00 | 70.13 | B |
| ATOM | 6050 | CA | PRO | B | 379 | −31.689 | 45.447 | 59.230 | 1.00 | 69.88 | B |
| ATOM | 6051 | CB | PRO | B | 379 | −30.337 | 44.863 | 58.814 | 1.00 | 69.78 | B |
| ATOM | 6052 | CG | PRO | B | 379 | −30.613 | 44.302 | 57.453 | 1.00 | 69.85 | B |
| ATOM | 6053 | C | PRO | B | 379 | −31.799 | 46.971 | 58.989 | 1.00 | 68.98 | B |
| ATOM | 6054 | O | PRO | B | 379 | −31.212 | 47.768 | 59.731 | 1.00 | 67.67 | B |
| ATOM | 6055 | N | ASP | B | 380 | −32.544 | 47.379 | 57.968 | 1.00 | 66.97 | B |
| ATOM | 6056 | CA | ASP | B | 380 | −32.694 | 48.803 | 57.706 | 1.00 | 67.27 | B |
| ATOM | 6057 | CB | ASP | B | 380 | −33.189 | 49.049 | 56.281 | 1.00 | 69.42 | B |
| ATOM | 6058 | CG | ASP | B | 380 | −34.491 | 48.328 | 55.982 | 1.00 | 72.08 | B |
| ATOM | 6059 | OD1 | ASP | B | 380 | −35.148 | 48.667 | 54.977 | 1.00 | 72.20 | B |
| ATOM | 6060 | OD2 | ASP | B | 380 | −34.853 | 47.408 | 56.745 | 1.00 | 73.93 | B |
| ATOM | 6061 | C | ASP | B | 380 | −33.692 | 49.406 | 58.681 | 1.00 | 67.06 | B |
| ATOM | 6062 | O | ASP | B | 380 | −33.753 | 50.622 | 58.858 | 1.00 | 68.62 | B |
| ATOM | 6063 | N | ARG | B | 381 | −34.475 | 48.549 | 59.321 | 1.00 | 65.36 | B |
| ATOM | 6064 | CA | ARG | B | 361 | −35.484 | 49.013 | 60.254 | 1.00 | 62.88 | B |
| ATOM | 6065 | CB | ARG | B | 381 | −36.507 | 47.903 | 60.532 | 1.00 | 61.60 | B |
| ATOM | 6066 | CG | ARG | B | 381 | −37.131 | 47.229 | 59.299 | 1.00 | 56.06 | B |
| ATOM | 6067 | CD | ARG | B | 381 | −38.220 | 48.049 | 58.675 | 1.00 | 53.90 | B |
| ATOM | 6068 | NE | ARG | B | 381 | −38.939 | 47.327 | 57.624 | 1.00 | 53.78 | B |
| ATOM | 6069 | CZ | ARG | B | 381 | −39.782 | 46.315 | 57.835 | 1.00 | 53.67 | B |
| ATOM | 6070 | NH1 | ARG | B | 381 | −40.026 | 45.884 | 59.070 | 1.00 | 53.08 | B |
| ATOM | 6071 | NH2 | ARG | B | 381 | −40.395 | 45.746 | 56.808 | 1.00 | 51.53 | B |
| ATOM | 6072 | C | ARG | B | 381 | −34.918 | 49.509 | 61.577 | 1.00 | 62.43 | B |
| ATOM | 6073 | O | ARG | B | 381 | −35.684 | 49.886 | 62.451 | 1.00 | 64.91 | B |
| ATOM | 6074 | N | ALA | B | 382 | −33.600 | 49.510 | 61.745 | 1.00 | 61.92 | B |
| ATOM | 6075 | CA | ALA | B | 382 | −33.006 | 49.988 | 63.001 | 1.00 | 62.30 | B |
| ATOM | 6076 | CB | ALA | B | 382 | −33.420 | 49.084 | 64.142 | 1.00 | 58.12 | B |
| ATOM | 6077 | C | ALA | B | 382 | −31.479 | 50.130 | 62.995 | 1.00 | 64.25 | B |
| ATOM | 6078 | O | ALA | B | 382 | −30.790 | 49.541 | 62.169 | 1.00 | 65.93 | B |
| ATOM | 6079 | N | PRO | B | 383 | −30.931 | 50.937 | 63.916 | 1.00 | 66.02 | B |
| ATOM | 6080 | CD | PRO | B | 383 | −31.640 | 51.850 | 64.828 | 1.00 | 67.48 | B |
| ATOM | 6081 | CA | PRO | B | 383 | −29.484 | 51.145 | 64.016 | 1.00 | 66.67 | B |
| ATOM | 6082 | CB | PRO | B | 383 | −29.357 | 52.020 | 65.255 | 1.00 | 66.55 | B |
| ATOM | 6083 | CG | PRO | B | 383 | −30.570 | 52.857 | 65.163 | 1.00 | 67.09 | B |
| ATOM | 6084 | C | PRO | B | 383 | −28.741 | 49.813 | 64.160 | 1.00 | 67.19 | B |
| ATOM | 6085 | O | PRO | B | 383 | −29.171 | 48.920 | 64.913 | 1.00 | 66.32 | B |
| ATOM | 6086 | N | ASN | B | 384 | −27.620 | 49.697 | 63.448 | 1.00 | 66.51 | B |
| ATOM | 6087 | CA | ASN | B | 384 | −26.832 | 48.478 | 63.457 | 1.00 | 66.05 | B |
| ATOM | 6088 | CB | ASN | B | 384 | −25.705 | 48.580 | 62.431 | 1.00 | 66.01 | B |
| ATOM | 6089 | CG | ASN | B | 384 | −24.832 | 49.794 | 62.639 | .00 | 66.54 | B |
| ATOM | 6090 | OD1 | ASN | B | 384 | −25.300 | 50.927 | 62.557 | .00 | 66.69 | B |
| ATOM | 6091 | ND2 | ASN | B | 384 | −23.553 | 49.565 | 62.905 | .00 | 66.71 | B |
| ATOM | 6092 | C | ASN | B | 384 | −26.275 | 48.038 | 64.809 | 1.00 | 66.27 | B |
| ATOM | 6093 | O | ASN | B | 384 | −25.731 | 46.939 | 64.918 | 1.00 | 67.03 | B |
| ATOM | 6094 | N | ASN | B | 385 | −26.423 | 48.862 | 65.844 | 1.00 | 65.45 | B |
| ATOM | 6095 | CA | ASN | B | 385 | −25.918 | 48.486 | 67.168 | 1.00 | 64.91 | B |
| ATOM | 6096 | CB | ASN | B | 385 | −25.126 | 49.624 | 67.777 | 1.00 | 66.89 | B |
| ATOM | 6097 | CG | ASN | B | 385 | −26.005 | 50.786 | 68.154 | 1.00 | 69.81 | B |
| ATOM | 6098 | OD1 | ASN | B | 385 | −26.651 | 51.398 | 67.293 | 1.00 | 71.78 | B |
| ATOM | 6099 | ND2 | ASN | B | 385 | −26.045 | 51.100 | 69.449 | 1.00 | 69.70 | B |
| ATOM | 6100 | C | ASN | B | 385 | −27.046 | 48.134 | 68.121 | 1.00 | 64.06 | B |
| ATOM | 6101 | O | ASN | B | 385 | −26.895 | 48.205 | 69.342 | 1.00 | 62.01 | B |
| ATOM | 6102 | N | VAL | B | 386 | −28.187 | 47.759 | 67.558 | 1.00 | 64.84 | B |
| ATOM | 6103 | CA | VAL | B | 386 | −29.341 | 47.401 | 68.377 | 1.00 | 64.08 | B |
| ATOM | 6104 | CB | VAL | B | 386 | −30.208 | 48.659 | 68.638 | 1.00 | 63.92 | B |
| ATOM | 6105 | CG1 | VAL | B | 386 | −30.824 | 49.154 | 67.341 | 1.00 | 63.13 | B |
| ATOM | 6106 | CG2 | VAL | B | 386 | −31.260 | 48.358 | 69.664 | 1.00 | 63.53 | B |
| ATOM | 6107 | C | VAL | B | 386 | −30.189 | 46.294 | 67.727 | 1.00 | 62.25 | B |
| ATOM | 6108 | O | VAL | B | 386 | −30.013 | 45.968 | 66.550 | 1.00 | 62.46 | B |
| ATOM | 6109 | N | TYR | B | 387 | −31.083 | 45.699 | 68.506 | 1.00 | 59.47 | B |
| ATOM | 6110 | CA | TYR | B | 387 | −31.962 | 44.655 | 67.999 | 1.00 | 58.51 | B |
| ATOM | 6111 | CB | TYR | B | 387 | −31.821 | 43.359 | 68.811 | 1.00 | 61.64 | B |
| ATOM | 6112 | CG | TYR | B | 387 | −30.604 | 42.502 | 68.508 | 1.00 | 64.73 | B |
| ATOM | 6113 | CD1 | TYR | B | 387 | −29.980 | 41.779 | 69.530 | 1.00 | 64.69 | B |
| ATOM | 6114 | CE1 | TYR | B | 387 | −28.882 | 40.965 | 69.272 | 1.00 | 64.47 | B |
| ATOM | 6115 | CD2 | TYR | B | 387 | −30.091 | 42.387 | 67.204 | 1.00 | 64.67 | B |
| ATOM | 6116 | CE2 | TYR | B | 387 | −28.988 | 41.570 | 66.935 | 1.00 | 64.74 | B |
| ATOM | 6117 | CZ | TYR | B | 387 | −28.392 | 40.862 | 67.978 | 1.00 | 65.13 | B |
| ATOM | 6118 | OH | TYR | B | 387 | −27.315 | 40.038 | 67.735 | 1.00 | 67.04 | B |
| ATOM | 6119 | C | TYR | B | 387 | −33.386 | 45.136 | 68.158 | 1.00 | 56.60 | B |
| ATOM | 6120 | O | TYR | B | 387 | −33.777 | 45.554 | 69.243 | 1.00 | 55.83 | B |
| ATOM | 6121 | N | LEU | B | 388 | −34.160 | 45.079 | 67.084 | 1.00 | 55.16 | B |
| ATOM | 6122 | CA | LEU | B | 388 | −35.564 | 45.470 | 67.142 | 1.00 | 54.71 | B |
| ATOM | 6123 | CB | LEU | B | 388 | −35.949 | 46.236 | 65.884 | 1.00 | 52.97 | B |
| ATOM | 6124 | CG | LEU | B | 388 | −37.413 | 46.656 | 65.861 | 1.00 | 51.30 | B |
| ATOM | 6125 | CD1 | LEU | B | 388 | −37.728 | 47.436 | 67.129 | 1.00 | 52.08 | B |
| ATOM | 6126 | CD2 | LEU | B | 388 | −37.687 | 47.484 | 64.622 | 1.00 | 51.76 | B |
| ATOM | 6127 | C | LEU | B | 388 | −36.429 | 44.206 | 67.252 | 1.00 | 55.61 | B |
| ATOM | 6128 | O | LEU | B | 388 | −36.440 | 43.375 | 66.336 | 1.00 | 56.76 | B |
| ATOM | 6129 | N | TYR | B | 389 | −37.148 | 44.057 | 68.362 | 1.00 | 54.95 | B |
| ATOM | 6130 | CA | TYR | B | 389 | −37.994 | 42.878 | 68.569 | 1.00 | 54.54 | B |
| ATOM | 6131 | CB | TYR | B | 389 | −37.624 | 42.182 | 69.892 | 1.00 | 55.70 | B |
| ATOM | 6132 | CG | TYR | B | 389 | −36.241 | 41.569 | 69.911 | 1.00 | 57.17 | B |
| ATOM | 6133 | CD1 | TYR | B | 389 | −35.518 | 41.463 | 71.106 | 1.00 | 58.98 | B |
| ATOM | 6134 | CE1 | TYR | B | 389 | −34.211 | 40.958 | 71.119 | 1.00 | 59.77 | B |
| ATOM | 6135 | CD2 | TYR | B | 389 | −35.633 | 41.142 | 68.732 | 1.00 | 56.70 | B |
| ATOM | 6136 | CE2 | TYR | B | 389 | −34.343 | 40.636 | 68.730 | 1.00 | 59.91 | B |
| ATOM | 6137 | CZ | TYR | B | 389 | −33.629 | 40.548 | 69.920 | 1.00 | 60.24 | B |
| ATOM | 6138 | OH | TYR | B | 389 | −32.334 | 40.072 | 69.879 | 1.00 | 59.24 | B |
| ATOM | 6139 | C | TYR | B | 389 | −39.490 | 43.181 | 68.575 | 1.00 | 53.44 | B |
| ATOM | 6140 | O | TYR | B | 389 | −39.956 | 44.011 | 69.355 | 1.00 | 55.49 | B |
| ATOM | 6141 | N | THR | B | 390 | −40.238 | 42.504 | 67.708 | 1.00 | 51.19 | B |
| ATOM | 6142 | CA | THR | B | 390 | −41.688 | 42.677 | 67.639 | 1.00 | 47.52 | B |
| ATOM | 6143 | CB | THR | B | 390 | −42.172 | 42.718 | 66.175 | 1.00 | 46.13 | B |
| ATOM | 6144 | OG1 | THR | B | 390 | −41.741 | 43.938 | 65.566 | 1.00 | 42.82 | B |
| ATOM | 6145 | CG2 | THR | B | 390 | −43.685 | 42.624 | 66.101 | 1.00 | 45.43 | B |
| ATOM | 6146 | C | THR | B | 390 | −42.344 | 41.501 | 68.368 | 1.00 | 47.27 | B |
| ATOM | 6147 | O | THR | B | 390 | −42.345 | 40.379 | 67.875 | 1.00 | 47.48 | B |
| ATOM | 6148 | N | THR | B | 391 | −42.889 | 41.759 | 69.552 | 1.00 | 46.40 | B |
| ATOM | 6149 | CA | THR | B | 391 | −43.529 | 40.710 | 70.335 | 1.00 | 46.48 | B |
| ATOM | 6150 | CB | THR | B | 391 | −43.007 | 40.704 | 71.761 | 1.00 | 45.84 | B |
| ATOM | 6151 | OG1 | THR | B | 391 | −41.612 | 40.403 | 71.748 | 1.00 | 47.01 | B |
| ATOM | 6152 | CG2 | THR | B | 391 | −43.739 | 39.670 | 72.592 | 1.00 | 45.04 | B |
| ATOM | 6153 | C | THR | B | 391 | −45.048 | 40.828 | 70.409 | 1.00 | 48.30 | B |
| ATOM | 6154 | O | THR | B | 391 | −45.594 | 41.922 | 70.552 | 1.00 | 50.22 | B |
| ATOM | 6155 | N | PHE | B | 392 | −45.729 | 39.690 | 70.327 | 1.00 | 47.98 | B |
| ATOM | 6156 | CA | PHE | B | 392 | −47.176 | 39.666 | 70.393 | 1.00 | 46.51 | B |
| ATOM | 6157 | CB | PHE | B | 392 | −47.718 | 38.803 | 69.266 | 1.00 | 44.95 | B |
| ATOM | 6158 | CG | PHE | B | 392 | −47.264 | 39.249 | 67.915 | 1.00 | 46.45 | B |
| ATOM | 6159 | CD1 | PHE | B | 392 | −46.048 | 38.833 | 67.402 | 1.00 | 47.37 | B |
| ATOM | 6160 | CD2 | PHE | B | 392 | −48.026 | 40.138 | 67.171 | 1.00 | 47.27 | B |
| ATOM | 6161 | CE1 | PHE | B | 392 | −45.591 | 39.295 | 66.169 | 1.00 | 45.41 | B |
| ATOM | 6162 | CE2 | PHE | B | 392 | −47.579 | 40.603 | 65.941 | 1.00 | 46.05 | B |
| ATOM | 6163 | CZ | PHE | B | 392 | −46.357 | 40.178 | 65.444 | 1.00 | 46.65 | B |
| ATOM | 6164 | C | PHE | B | 392 | −47.582 | 39.110 | 71.742 | 1.00 | 47.34 | B |
| ATOM | 6165 | O | PHE | B | 392 | −47.147 | 38.027 | 72.122 | 1.00 | 48.37 | B |
| ATOM | 6166 | N | VAL | B | 393 | −48.407 | 39.860 | 72.467 | 1.00 | 47.59 | B |
| ATOM | 6167 | CA | VAL | B | 393 | −48.869 | 39.453 | 73.796 | 1.00 | 47.43 | B |
| ATOM | 6168 | CB | VAL | B | 393 | −48.498 | 40.490 | 74.865 | 1.00 | 45.93 | B |
| ATOM | 6169 | CG1 | VAL | B | 393 | −48.802 | 39.930 | 76.238 | 1.00 | 45.57 | B |
| ATOM | 6170 | CG2 | VAL | B | 393 | −47.056 | 40.900 | 74.731 | 1.00 | 43.00 | B |
| ATOM | 6171 | C | VAL | B | 393 | −50.383 | 39.320 | 73.846 | 1.00 | 48.87 | B |
| ATOM | 6172 | O | VAL | B | 393 | −51.093 | 40.025 | 73.131 | 1.00 | 50.76 | B |
| ATOM | 6173 | N | GLY | B | 394 | −50.869 | 38.440 | 74.716 | 1.00 | 49.72 | B |
| ATOM | 6174 | CA | GLY | B | 394 | −52.302 | 38.236 | 74.857 | 1.00 | 49.47 | B |
| ATOM | 6175 | C | GLY | B | 394 | −52.813 | 36.957 | 74.217 | 1.00 | 50.58 | B |
| ATOM | 6176 | O | GLY | B | 394 | −52.435 | 35.849 | 74.610 | 1.00 | 48.81 | B |
| ATOM | 6177 | N | GLY | B | 395 | −53.684 | 37.112 | 73.221 | 1.00 | 51.86 | B |
| ATOM | 6178 | CA | GLY | B | 395 | −54.238 | 35.956 | 72.537 | 1.00 | 51.27 | B |
| ATOM | 6179 | C | GLY | B | 395 | −55.193 | 35.162 | 73.407 | 1.00 | 51.12 | B |
| ATOM | 6180 | O | GLY | B | 395 | −55.345 | 35.424 | 74.596 | 1.00 | 49.91 | B |
| ATOM | 6181 | N | SER | B | 396 | −55.831 | 34.170 | 72.806 | 1.00 | 52.21 | B |
| ATOM | 6182 | CA | SER | B | 396 | −56.790 | 33.332 | 73.508 | 1.00 | 52.40 | B |
| ATOM | 6183 | CB | SER | B | 396 | −57.244 | 32.198 | 72.587 | 1.00 | 51.94 | B |
| ATOM | 6184 | OG | SER | B | 396 | −57.768 | 32.697 | 71.364 | 1.00 | 51.44 | B |
| ATOM | 6185 | C | SER | B | 396 | −56.260 | 32.751 | 74.820 | 1.00 | 53.37 | B |
| ATOM | 6186 | O | SER | B | 396 | −57.003 | 32.597 | 75.780 | 1.00 | 54.67 | B |
| ATOM | 6187 | N | ARG | B | 397 | −54.976 | 32.430 | 74.878 | 1.00 | 54.94 | B |
| ATOM | 6188 | CA | ARG | B | 397 | −54.432 | 31.849 | 76.101 | 1.00 | 56.15 | B |
| ATOM | 6189 | CB | ARG | B | 397 | −53.021 | 31.311 | 75.866 | 1.00 | 55.46 | B |
| ATOM | 6190 | CG | ARG | B | 397 | −51.953 | 32.367 | 75.888 | 1.00 | 52.27 | B |
| ATOM | 6191 | CD | ARG | B | 397 | −50.670 | 31.800 | 75.381 | 1.00 | 51.86 | B |
| ATOM | 6192 | NE | ARG | B | 397 | −50.792 | 31.313 | 74.012 | 1.00 | 49.97 | B |
| ATOM | 6193 | CZ | ARG | B | 397 | −49.777 | 30.794 | 73.333 | 1.00 | 51.58 | B |
| ATOM | 6194 | NH1 | ARG | B | 397 | −48.581 | 30.706 | 73.907 | 1.00 | 53.40 | B |
| ATOM | 6195 | NH2 | ARG | B | 397 | −49.949 | 30.361 | 72.091 | 1.00 | 51.53 | B |
| ATOM | 6196 | C | ARG | B | 397 | −54.390 | 32.840 | 77.248 | 1.00 | 57.37 | B |
| ATOM | 6197 | O | ARG | B | 397 | −54.185 | 32.457 | 78.393 | 1.00 | 58.78 | B |
| ATOM | 6198 | N | ASN | B | 398 | −54.569 | 34.118 | 76.947 | 1.00 | 58.80 | B |
| ATOM | 6199 | CA | ASN | B | 398 | −54.543 | 35.123 | 78.000 | 1.00 | 60.00 | B |
| ATOM | 6200 | CB | ASN | B | 398 | −53.138 | 35.184 | 78.585 | 1.00 | 60.61 | B |
| ATOM | 6201 | CG | ASN | B | 398 | −53.082 | 35.971 | 79.861 | 1.00 | 62.55 | B |
| ATOM | 6202 | OD1 | ASN | B | 398 | −54.091 | 36.127 | 80.551 | 1.00 | 63.50 | B |
| ATOM | 6203 | ND2 | ASN | B | 398 | −51.895 | 36.466 | 80.198 | 1.00 | 63.85 | B |
| ATOM | 6204 | C | ASN | B | 398 | −55.014 | 36.499 | 77.506 | 1.00 | 60.89 | B |
| ATOM | 6205 | O | ASN | B | 398 | −54.223 | 37.360 | 77.117 | 1.00 | 60.06 | B |
| ATOM | 6206 | N | ARG | B | 399 | −56.331 | 36.676 | 77.533 | 1.00 | 62.25 | B |
| ATOM | 6207 | CA | ARG | B | 399 | −56.997 | 37.891 | 77.082 | 1.00 | 62.34 | B |
| ATOM | 6208 | CB | ARG | B | 399 | −58.507 | 37.658 | 77.057 | 1.00 | 59.96 | B |
| ATOM | 6209 | CG | ARG | B | 399 | −58.928 | 36.599 | 76.092 | 1.00 | 56.80 | B |
| ATOM | 6210 | CD | ARG | B | 399 | −60.416 | 36.453 | 76.071 | 1.00 | 55.98 | B |
| ATOM | 6211 | NE | ARG | B | 399 | −60.821 | 35.665 | 74.918 | 1.00 | 57.45 | B |
| ATOM | 6212 | CZ | ARG | B | 399 | −61.358 | 36.179 | 73.819 | 1.00 | 58.45 | B |
| ATOM | 6213 | NH1 | ARG | B | 399 | −61.562 | 37.489 | 73.733 | 1.00 | 56.87 | B |
| ATOM | 6214 | NH2 | ARG | B | 399 | −61.674 | 35.384 | 72.803 | 1.00 | 59.75 | B |
| ATOM | 6215 | C | ARG | B | 399 | −56.705 | 39.119 | 77.921 | 1.00 | 63.67 | B |
| ATOM | 6216 | O | ARG | B | 399 | −56.486 | 40.206 | 77.388 | 1.00 | 62.89 | B |
| ATOM | 6217 | N | GLU | B | 400 | −56.723 | 38.947 | 79.235 | 1.00 | 65.37 | B |
| ATOM | 6218 | CA | GLU | B | 400 | −56.474 | 40.059 | 80.127 | 1.00 | 68.72 | B |
| ATOM | 6219 | CB | GLU | B | 400 | −57.015 | 39.756 | 81.528 | 1.00 | 71.84 | B |
| ATOM | 6220 | CG | GLU | B | 400 | −58.538 | 39.876 | 81.648 | 1.00 | 76.04 | B |
| ATOM | 6221 | CD | GLU | B | 400 | −59.082 | 41.211 | 81.121 | 1.00 | 77.81 | B |
| ATOM | 6222 | OE1 | GLU | B | 400 | −58.550 | 42.281 | 81.516 | 1.00 | 76.87 | B |
| ATOM | 6223 | OE2 | GLU | B | 400 | −60.050 | 41.180 | 80.319 | 1.00 | 78.76 | B |
| ATOM | 6224 | C | GLU | B | 400 | −55.006 | 40.425 | 80.219 | 1.00 | 68.94 | B |
| ATOM | 6225 | O | GLU | B | 400 | −54.546 | 40.925 | 81.248 | 1.00 | 72.14 | B |
| ATOM | 6226 | N | LEU | B | 401 | −54.260 | 40.173 | 79.154 | 1.00 | 67.32 | B |
| ATOM | 6227 | CA | LEU | B | 401 | −52.848 | 40.508 | 79.150 | 1.00 | 65.62 | B |
| ATOM | 6228 | CB | LEU | B | 401 | −52.010 | 39.250 | 78.980 | 1.00 | 65.30 | B |
| ATOM | 6229 | CG | LEU | B | 401 | −50.528 | 39.461 | 79.254 | 1.00 | 66.27 | B |
| ATOM | 6230 | CD1 | LEU | B | 401 | −50.364 | 39.834 | 80.711 | 1.00 | 65.75 | B |
| ATOM | 6231 | CD2 | LEU | B | 401 | −49.741 | 38.197 | 78.925 | 1.00 | 66.81 | B |
| ATOM | 6232 | C | LEU | B | 401 | −52.617 | 41.456 | 77.983 | 1.00 | 65.38 | B |
| ATOM | 6233 | O | LEU | B | 401 | −51.693 | 42.260 | 77.981 | 1.00 | 65.74 | B |
| ATOM | 6234 | N | ALA | B | 402 | −53.483 | 41.353 | 76.986 | 1.00 | 64.71 | B |
| ATOM | 6235 | CA | ALA | B | 402 | −53.401 | 42.191 | 75.807 | 1.00 | 63.15 | B |
| ATOM | 6236 | CB | ALA | B | 402 | −54.329 | 41.642 | 74.741 | 1.00 | 61.42 | B |
| ATOM | 6237 | C | ALA | B | 402 | −53.794 | 43.620 | 76.178 | 1.00 | 63.62 | B |
| ATOM | 6238 | O | ALA | B | 402 | −53.424 | 44.576 | 75.494 | 1.00 | 62.66 | B |
| ATOM | 6239 | N | LYS | B | 403 | −54.531 | 43.751 | 77.281 | 1.00 | 64.92 | B |
| ATOM | 6240 | CA | LYS | B | 403 | −55.009 | 45.044 | 77.763 | 1.00 | 65.45 | B |
| ATOM | 6241 | CB | LYS | B | 403 | −56.457 | 44.912 | 78.236 | 1.00 | 66.31 | B |
| ATOM | 6242 | CG | LYS | B | 403 | −57.396 | 44.299 | 77.207 | 1.00 | 66.65 | B |
| ATOM | 6243 | CD | LYS | B | 403 | −58.854 | 44.476 | 77.607 | 1.00 | 67.78 | B |
| ATOM | 6244 | CE | LYS | B | 403 | −59.135 | 43.928 | 79.006 | 1.00 | 68.65 | B |
| ATOM | 6245 | NZ | LYS | B | 403 | −60.600 | 43.911 | 79.333 | 1.00 | 69.29 | B |
| ATOM | 6246 | C | LYS | B | 403 | −54.174 | 45.648 | 78.892 | 1.00 | 65.67 | B |
| ATOM | 6247 | O | LYS | B | 403 | −54.587 | 46.621 | 79.521 | 1.00 | 66.32 | B |
| ATOM | 6248 | N | ALA | B | 404 | −53.003 | 45.073 | 79.152 | 1.00 | 66.11 | B |
| ATOM | 6249 | CA | ALA | B | 404 | −52.118 | 45.566 | 80.209 | 1.00 | 64.06 | B |
| ATOM | 6250 | CB | ALA | B | 404 | −50.990 | 44.570 | 80.453 | 1.00 | 63.29 | B |
| ATOM | 6251 | C | ALA | B | 404 | −51.545 | 46.922 | 79.834 | 1.00 | 63.19 | B |
| ATOM | 6252 | O | ALA | B | 404 | −51.560 | 47.305 | 78.657 | 1.00 | 62.18 | B |
| ATOM | 6253 | N | SER | B | 405 | −51.045 | 47.643 | 80.839 | 1.00 | 63.28 | B |
| ATOM | 6254 | CA | SER | B | 405 | −50.458 | 48.973 | 80.631 | 1.00 | 63.95 | B |
| ATOM | 6255 | CB | SER | B | 405 | −50.208 | 49.678 | 81.968 | 1.00 | 63.38 | B |
| ATOM | 6256 | OG | SER | B | 405 | −49.241 | 48.988 | 82.738 | 1.00 | 64.29 | B |
| ATOM | 6257 | C | SER | B | 405 | −49.142 | 48.819 | 79.892 | 1.00 | 63.82 | B |
| ATOM | 6258 | O | SER | B | 405 | −48.494 | 47.772 | 79.989 | 1.00 | 64.21 | B |
| ATOM | 6259 | N | ARG | B | 406 | −48.736 | 49.843 | 79.149 | 1.00 | 62.44 | B |
| ATOM | 6260 | CA | ARG | B | 406 | −47.487 | 49.711 | 78.431 | 1.00 | 62.18 | B |
| ATOM | 6261 | CB | ARG | B | 406 | −47.102 | 51.012 | 77.732 | 1.00 | 61.11 | B |
| ATOM | 6262 | CG | ARG | B | 406 | −45.754 | 50.939 | 77.027 | 1.00 | 59.23 | B |
| ATOM | 6263 | CD | ARG | B | 406 | −45.570 | 52.112 | 76.094 | 1.00 | 59.98 | B |
| ATOM | 6264 | NE | ARG | B | 406 | −46.582 | 52.110 | 75.047 | 1.00 | 59.55 | B |
| ATOM | 6265 | CZ | ARG | B | 406 | −46.689 | 53.040 | 74.107 | 1.00 | 60.93 | B |
| ATOM | 6266 | NH1 | ARG | B | 406 | −45.847 | 54.062 | 74.078 | 1.00 | 62.31 | B |
| ATOM | 6267 | NH2 | ARG | B | 406 | −47.636 | 52.944 | 73.187 | 1.00 | 60.83 | B |
| ATOM | 6268 | C | ARG | B | 406 | −46.391 | 49.282 | 79.400 | 1.00 | 62.72 | B |
| ATOM | 6269 | O | ARG | B | 406 | −45.487 | 48.550 | 79.016 | 1.00 | 62.87 | B |
| ATOM | 6270 | N | THR | B | 407 | −46.478 | 49.713 | 80.657 | 1.00 | 62.88 | B |
| ATOM | 6271 | CA | THR | B | 407 | −45.465 | 49.331 | 81.638 | 1.00 | 62.82 | B |
| ATOM | 6272 | CB | THR | B | 407 | −45.637 | 50.075 | 82.996 | 1.00 | 62.61 | B |
| ATOM | 6273 | OG1 | THR | B | 407 | −45.512 | 51.490 | 82.797 | 1.00 | 61.89 | B |
| ATOM | 6274 | CG2 | THR | B | 407 | −44.556 | 49.640 | 83.980 | 1.00 | 61.53 | B |
| ATOM | 6275 | C | THR | B | 407 | −45.537 | 47.826 | 81.869 | 1.00 | 63.07 | B |
| ATOM | 6276 | O | THR | B | 407 | −44.567 | 47.109 | 81.607 | 1.00 | 63.41 | B |
| ATOM | 6277 | N | GLU | B | 408 | −46.678 | 47.340 | 82.351 | 1.00 | 63.06 | B |
| ATOM | 6278 | CA | GLU | B | 408 | −46.834 | 45.906 | 82.575 | 1.00 | 64.62 | B |
| ATOM | 6279 | CB | GLU | B | 408 | −48.286 | 45.576 | 82.932 | 1.00 | 65.87 | B |
| ATOM | 6280 | CG | GLU | B | 408 | −48.564 | 45.583 | 84.415 | 1.00 | 68.45 | B |
| ATOM | 6281 | CD | GLU | B | 408 | −49.972 | 46.050 | 84.753 | 1.00 | 71.53 | B |
| ATOM | 6282 | OE1 | GLU | B | 408 | −50.318 | 46.066 | 85.957 | 1.00 | 73.48 | B |
| ATOM | 6283 | OE2 | GLU | B | 408 | −50.733 | 46.411 | 83.825 | 1.00 | 72.29 | B |
| ATOM | 6284 | C | GLU | B | 408 | −46.409 | 45.120 | 81.327 | 1.00 | 64.53 | B |
| ATOM | 6285 | O | GLU | B | 408 | −45.788 | 44.061 | 81.436 | 1.00 | 64.16 | B |
| ATOM | 6286 | N | LEU | B | 409 | −46.738 | 45.647 | 80.146 | 1.00 | 63.16 | B |
| ATOM | 6287 | CA | LEU | B | 409 | −46.379 | 44.993 | 78.898 | 1.00 | 61.03 | B |
| ATOM | 6288 | CB | LEU | B | 409 | −47.061 | 45.673 | 77.716 | 1.00 | 60.73 | B |
| ATOM | 6289 | CG | LEU | B | 409 | −48.558 | 45.437 | 77.528 | 1.00 | 59.95 | B |
| ATOM | 6290 | CD1 | LEU | B | 409 | −49.057 | 46.362 | 76.456 | 1.00 | 60.85 | B |
| ATOM | 6291 | CD2 | LEU | B | 409 | −48.829 | 44.002 | 77.144 | 1.00 | 57.72 | B |
| ATOM | 6292 | C | LEU | B | 409 | −44.878 | 45.024 | 78.695 | 1.00 | 60.49 | B |
| ATOM | 6293 | O | LEU | B | 409 | −44.305 | 44.070 | 78.188 | 1.00 | 62.30 | B |
| ATOM | 6294 | N | LYS | B | 410 | −44.237 | 46.118 | 79.090 | 1.00 | 59.61 | B |
| ATOM | 6295 | CA | LYS | B | 410 | −42.787 | 46.247 | 78.941 | 1.00 | 58.22 | B |
| ATOM | 6296 | CB | LYS | B | 410 | −42.328 | 47.629 | 79.389 | 1.00 | 58.57 | B |
| ATOM | 6297 | CG | LYS | B | 410 | −40.852 | 47.882 | 79.220 | 1.00 | 58.88 | B |
| ATOM | 6298 | CD | LYS | B | 410 | −40.426 | 49.143 | 79.990 | 1.00 | 62.24 | B |
| ATOM | 6299 | CE | LYS | B | 410 | −38.923 | 49.391 | 79.874 | 1.00 | 62.94 | B |
| ATOM | 6300 | NZ | LYS | B | 410 | −38.437 | 50.527 | 80.692 | 1.00 | 62.98 | B |
| ATOM | 6301 | C | LYS | B | 410 | −42.075 | 45.183 | 79.769 | 1.00 | 57.72 | B |
| ATOM | 6302 | O | LYS | B | 410 | −41.053 | 44.643 | 79.346 | 1.00 | 58.11 | B |
| ATOM | 6303 | N | GLU | B | 411 | −42.601 | 44.880 | 80.953 | 1.00 | 55.81 | B |
| ATOM | 6304 | CA | GLU | B | 411 | −41.979 | 43.857 | 81.779 | 1.00 | 55.31 | B |
| ATOM | 6305 | CB | GLU | B | 411 | −42.504 | 43.912 | 83.212 | 1.00 | 54.86 | B |
| ATOM | 6306 | CG | GLU | B | 411 | −41.760 | 44.895 | 84.093 | 1.00 | 56.27 | B |
| ATOM | 6307 | CD | GLU | B | 411 | −40.277 | 44.574 | 84.204 | .00 | 56.19 | B |
| ATOM | 6308 | OE1 | GLU | B | 411 | −39.940 | 43.482 | 84.707 | .00 | 56.43 | B |
| ATOM | 6309 | OE2 | GLU | B | 411 | −39.449 | 45.412 | 83.787 | .00 | 56.43 | B |
| ATOM | 6310 | C | GLU | B | 411 | −42.199 | 42.466 | 81.196 | 1.00 | 55.71 | B |
| ATOM | 6311 | O | GLU | B | 411 | −41.245 | 41.720 | 81.054 | 1.00 | 56.74 | B |
| ATOM | 6312 | N | ILE | B | 412 | −43.449 | 42.130 | 80.863 | 1.00 | 55.43 | B |
| ATOM | 6313 | CA | ILE | B | 412 | −43.806 | 40.833 | 80.280 | 1.00 | 54.40 | B |
| ATOM | 6314 | CB | ILE | B | 412 | −45.265 | 40.830 | 79.748 | 1.00 | 55.54 | B |
| ATOM | 6315 | CG2 | ILE | B | 412 | −45.614 | 39.454 | 79.162 | 1.00 | 54.48 | B |
| ATOM | 6316 | CG1 | ILE | B | 412 | −46.240 | 41.154 | 80.876 | 1.00 | 55.83 | B |
| ATOM | 6317 | CD1 | ILE | B | 412 | −46.475 | 40.004 | 81.825 | 1.00 | 57.87 | B |
| ATOM | 6318 | C | ILE | B | 412 | −42.896 | 40.488 | 79.100 | 1.00 | 53.86 | B |
| ATOM | 6319 | O | ILE | B | 412 | −42.328 | 39.389 | 79.033 | 1.00 | 51.52 | B |
| ATOM | 6320 | N | VAL | B | 413 | −42.779 | 41.419 | 78.158 | 1.00 | 52.24 | B |
| ATOM | 6321 | CA | VAL | B | 413 | −41.930 | 41.185 | 77.006 | 1.00 | 53.66 | B |
| ATOM | 6322 | CB | VAL | B | 413 | −42.002 | 42.350 | 75.992 | 1.00 | 51.98 | B |
| ATOM | 6323 | CG1 | VAL | B | 413 | −40.784 | 42.333 | 75.083 | 1.00 | 49.83 | B |
| ATOM | 6324 | CG2 | VAL | B | 413 | −43.259 | 42.213 | 75.143 | 1.00 | 49.15 | B |
| ATOM | 6325 | C | VAL | B | 413 | −40.488 | 40.970 | 77.450 | 1.00 | 55.62 | B |
| ATOM | 6326 | O | VAL | B | 413 | −39.850 | 40.006 | 77.045 | 1.00 | 57.66 | B |
| ATOM | 6327 | N | THR | B | 414 | −39.977 | 41.853 | 78.297 | 1.00 | 57.18 | B |
| ATOM | 6328 | CA | THR | B | 414 | −38.607 | 41.724 | 78.775 | 1.00 | 58.03 | B |
| ATOM | 6329 | CB | THR | B | 414 | −38.255 | 42.849 | 79.776 | 1.00 | 58.68 | B |
| ATOM | 6330 | OG1 | THR | B | 414 | −38.140 | 44.092 | 79.073 | 1.00 | 58.48 | B |
| ATOM | 6331 | CG2 | THR | B | 414 | −36.930 | 42.548 | 80.482 | 1.00 | 59.24 | B |
| ATOM | 6332 | C | THR | B | 414 | −38.355 | 40.380 | 79.449 | 1.00 | 57.44 | B |
| ATOM | 6333 | O | THR | B | 414 | −37.351 | 39.730 | 79.212 | 1.00 | 58.38 | B |
| ATOM | 6334 | N | SER | B | 415 | −39.276 | 39.964 | 80.292 | 1.00 | 57.89 | B |
| ATOM | 6335 | CA | SER | B | 415 | −39.119 | 38.716 | 81.014 | 1.00 | 59.38 | B |
| ATOM | 6336 | CB | SER | B | 415 | −40.360 | 38.451 | 81.862 | 1.00 | 59.13 | B |
| ATOM | 6337 | OG | SER | B | 415 | −40.146 | 37.365 | 82.734 | 1.00 | 60.31 | B |
| ATOM | 6338 | C | SER | B | 415 | −38.878 | 37.538 | 80.088 | 1.00 | 60.93 | B |
| ATOM | 6339 | O | SER | B | 415 | −37.988 | 36.713 | 80.335 | 1.00 | 61.79 | B |
| ATOM | 6340 | N | ASP | B | 416 | −39.683 | 37.455 | 79.030 | 1.00 | 61.36 | B |
| ATOM | 6341 | CA | ASP | B | 416 | −39.569 | 36.367 | 78.062 | 1.00 | 60.31 | B |
| ATOM | 6342 | CB | ASP | B | 416 | −40.827 | 36.297 | 77.185 | 1.00 | 62.01 | B |
| ATOM | 6343 | CG | ASP | B | 416 | −42.019 | 35.678 | 77.919 | 1.00 | 65.08 | B |
| ATOM | 6344 | OD1 | ASP | B | 416 | −43.038 | 35.379 | 77.252 | 1.00 | 66.16 | B |
| ATOM | 6345 | OD2 | ASP | B | 416 | −41.940 | 35.490 | 79.161 | 1.00 | 65.71 | B |
| ATOM | 6346 | C | ASP | B | 416 | −38.324 | 36.483 | 77.189 | 1.00 | 58.31 | B |
| ATOM | 6347 | O | ASP | B | 416 | −37.677 | 35.481 | 76.895 | 1.00 | 58.49 | B |
| ATOM | 6348 | N | LEU | B | 417 | −37.988 | 37.699 | 76.779 | 1.00 | 54.96 | B |
| ATOM | 6349 | CA | LEU | B | 417 | −36.814 | 37.914 | 75.958 | 1.00 | 53.08 | B |
| ATOM | 6350 | CB | LEU | B | 417 | −36.768 | 39.356 | 75.488 | 1.00 | 50.06 | B |
| ATOM | 6351 | CG | LEU | B | 417 | −37.801 | 39.601 | 74.407 | 1.00 | 50.61 | B |
| ATOM | 6352 | CD1 | LEU | B | 417 | −37.700 | 41.015 | 73.894 | 1.00 | 51.42 | B |
| ATOM | 6353 | CD2 | LEU | B | 417 | −37.564 | 38.613 | 73.284 | 1.00 | 50.81 | B |
| ATOM | 6354 | C | LEU | B | 417 | −35.534 | 37.572 | 76.712 | 1.00 | 54.94 | B |
| ATOM | 6355 | O | LEU | B | 417 | −34.536 | 37.160 | 76.123 | 1.00 | 55.05 | B |
| ATOM | 6356 | N | LYS | B | 418 | −35.557 | 37.749 | 78.022 | 1.00 | 56.33 | B |
| ATOM | 6357 | CA | LYS | B | 418 | −34.389 | 37.438 | 78.824 | 1.00 | 57.61 | B |
| ATOM | 6358 | CB | LYS | B | 418 | −34.623 | 37.875 | 80.274 | 1.00 | 57.14 | B |
| ATOM | 6359 | CG | LYS | B | 418 | −33.398 | 37.806 | 81.156 | 1.00 | 55.70 | B |
| ATOM | 6360 | CD | LYS | B | 418 | −33.763 | 37.941 | 82.619 | 1.00 | 56.18 | B |
| ATOM | 6361 | CE | LYS | B | 418 | −32.524 | 37.856 | 83.494 | 1.00 | 56.20 | B |
| ATOM | 6362 | NZ | LYS | B | 418 | −32.864 | 37.767 | 84.940 | 1.00 | 59.26 | B |
| ATOM | 6363 | C | LYS | B | 418 | −34.228 | 35.916 | 78.744 | 1.00 | 59.40 | B |
| ATOM | 6364 | O | LYS | B | 418 | −33.227 | 35.396 | 78.237 | 1.00 | 59.94 | B |
| ATOM | 6365 | N | GLN | B | 419 | −35.243 | 35.208 | 79.227 | 1.00 | 60.07 | B |
| ATOM | 6366 | CA | GLN | B | 419 | −35.242 | 33.750 | 79.236 | 1.00 | 60.60 | B |
| ATOM | 6367 | CB | GLN | B | 419 | −36.525 | 33.254 | 79.909 | 1.00 | 60.92 | B |
| ATOM | 6368 | CG | GLN | B | 419 | −36.794 | 31.772 | 79.777 | 1.00 | 63.81 | B |
| ATOM | 6369 | CD | GLN | B | 419 | −38.015 | 31.334 | 80.581 | 1.00 | 67.03 | B |
| ATOM | 6370 | OE1 | GLN | B | 419 | −39.007 | 32.063 | 80.677 | 1.00 | 67.45 | B |
| ATOM | 6371 | NE2 | GLN | B | 419 | −37.952 | 30.130 | 81.153 | 1.00 | 68.09 | B |
| ATOM | 6372 | C | GLN | B | 419 | −35.090 | 33.100 | 77.855 | 1.00 | 60.05 | B |
| ATOM | 6373 | O | GLN | B | 419 | −34.610 | 31.976 | 77.743 | 1.00 | 60.63 | B |
| ATOM | 6374 | N | LEU | B | 420 | −35.489 | 33.794 | 76.800 | 1.00 | 59.17 | B |
| ATOM | 6375 | CA | LEU | B | 420 | −35.393 | 33.199 | 75.482 | 1.00 | 58.73 | B |
| ATOM | 6376 | CB | LEU | B | 420 | −36.598 | 33.592 | 74.634 | 1.00 | 58.70 | B |
| ATOM | 6377 | CG | LEU | B | 420 | −37.944 | 32.951 | 74.980 | 1.00 | 58.86 | B |
| ATOM | 6378 | CD1 | LEU | B | 420 | −38.997 | 33.535 | 74.067 | 1.00 | 58.73 | B |
| ATOM | 6379 | CD2 | LEU | B | 420 | −37.889 | 31.437 | 74.811 | 1.00 | 57.87 | B |
| ATOM | 6380 | C | LEU | B | 420 | −34.121 | 33.567 | 74.755 | 1.00 | 59.85 | B |
| ATOM | 6381 | O | LEU | B | 420 | −33.451 | 32.699 | 74.187 | 1.00 | 59.59 | B |
| ATOM | 6382 | N | LEU | B | 421 | −33.778 | 34.851 | 74.773 | 1.00 | 59.92 | B |
| ATOM | 6383 | CA | LEU | B | 421 | −32.574 | 35.303 | 74.089 | 1.00 | 60.00 | B |
| ATOM | 6384 | CB | LEU | B | 421 | −32.925 | 36.430 | 73.125 | 1.00 | 57.12 | B |
| ATOM | 6385 | CG | LEU | B | 421 | −34.179 | 36.256 | 72.286 | 1.00 | 55.89 | B |
| ATOM | 6386 | CD1 | LEU | B | 421 | −34.177 | 37.280 | 71.161 | 1.00 | 54.88 | B |
| ATOM | 6387 | CD2 | LEU | B | 421 | −34.220 | 34.861 | 71.724 | 1.00 | 58.46 | B |
| ATOM | 6388 | C | LEU | B | 421 | −31.460 | 35.790 | 75.013 | 1.00 | 61.85 | B |
| ATOM | 6389 | O | LEU | B | 421 | −30.422 | 36.244 | 74.534 | 1.00 | 61.76 | B |
| ATOM | 6390 | N | GLY | B | 422 | −31.658 | 35.693 | 76.324 | 1.00 | 63.75 | B |
| ATOM | 6391 | CA | GLY | B | 422 | −30.651 | 36.199 | 77.236 | 1.00 | 66.80 | B |
| ATOM | 6392 | C | GLY | B | 422 | −30.603 | 37.718 | 77.078 | 1.00 | 70.57 | B |
| ATOM | 6393 | O | GLY | B | 422 | −29.537 | 38.303 | 76.838 | 1.00 | 71.30 | B |
| ATOM | 6394 | N | ALA | B | 423 | −31.764 | 38.368 | 77.176 | 1.00 | 72.31 | B |
| ATOM | 6395 | CA | ALA | B | 423 | −31.825 | 39.821 | 77.043 | 1.00 | 73.73 | B |
| ATOM | 6396 | CB | ALA | B | 423 | −33.191 | 40.253 | 76.535 | 1.00 | 72.18 | B |
| ATOM | 6397 | C | ALA | B | 423 | −31.548 | 40.421 | 78.413 | 1.00 | 74.59 | B |
| ATOM | 6398 | O | ALA | B | 423 | −32.185 | 40.056 | 79.402 | 1.00 | 73.40 | B |
| ATOM | 6399 | N | GLU | B | 424 | −30.584 | 41.334 | 78.465 | 1.00 | 76.35 | B |
| ATOM | 6400 | CA | GLU | B | 424 | −30.202 | 41.964 | 79.721 | 1.00 | 77.42 | B |
| ATOM | 6401 | CB | GLU | B | 424 | −28.672 | 41.907 | 79.873 | 1.00 | 79.22 | B |
| ATOM | 6402 | CG | GLU | B | 424 | −28.101 | 40.488 | 79.969 | 1.00 | 82.07 | B |
| ATOM | 6403 | CD | GLU | B | 424 | −26.571 | 40.449 | 79.878 | 1.00 | 84.69 | B |
| ATOM | 6404 | OE1 | GLU | B | 424 | −26.022 | 40.893 | 78.839 | 1.00 | 84.99 | B |
| ATOM | 6405 | OE2 | GLU | B | 424 | −25.917 | 39.971 | 80.839 | 1.00 | 85.03 | B |
| ATOM | 6406 | C | GLU | B | 424 | −30.693 | 43.410 | 79.902 | 1.00 | 76.33 | B |
| ATOM | 6407 | O | GLU | B | 424 | −30.486 | 44.282 | 79.043 | 1.00 | 74.78 | B |
| ATOM | 6408 | N | GLY | B | 425 | −31.351 | 43.648 | 81.032 | 1.00 | 74.87 | B |
| ATOM | 6409 | CA | GLY | B | 425 | −31.835 | 44.980 | 81.337 | 1.00 | 74.95 | B |
| ATOM | 6410 | C | GLY | B | 425 | −33.153 | 45.378 | 80.701 | 1.00 | 74.79 | B |
| ATOM | 6411 | O | GLY | B | 425 | −33.921 | 44.523 | 80.247 | 1.00 | 75.90 | B |
| ATOM | 6412 | N | GLU | B | 426 | −33.411 | 46.686 | 80.678 | 1.00 | 72.28 | B |
| ATOM | 6413 | CA | GLU | B | 426 | −34.633 | 47.222 | 80.104 | 1.00 | 70.17 | B |
| ATOM | 6414 | CB | GLU | B | 426 | −35.040 | 48.507 | 80.828 | 1.00 | 74.47 | B |
| ATOM | 6415 | CG | GLU | B | 426 | −35.346 | 48.322 | 82.310 | 1.00 | 78.38 | B |
| ATOM | 6416 | CD | GLU | B | 426 | −36.280 | 47.154 | 82.568 | 1.00 | 81.11 | B |
| ATOM | 6417 | OE1 | GLU | B | 426 | −35.847 | 45.997 | 82.374 | 1.00 | 82.42 | B |
| ATOM | 6418 | OE2 | GLU | B | 426 | −37.446 | 47.391 | 82.960 | 1.00 | 83.62 | B |
| ATOM | 6419 | C | GLU | B | 426 | −34.505 | 47.482 | 78.603 | 1.00 | 67.15 | B |
| ATOM | 6420 | O | GLU | B | 426 | −33.423 | 47.742 | 78.088 | 1.00 | 66.73 | B |
| ATOM | 6421 | N | PRO | B | 427 | −35.629 | 47.420 | 77.890 | 1.00 | 63.90 | B |
| ATOM | 6422 | CD | PRO | B | 427 | −36.922 | 47.067 | 78.497 | 1.00 | 63.79 | B |
| ATOM | 6423 | CA | PRO | B | 427 | −35.795 | 47.612 | 76.457 | 1.00 | 62.91 | B |
| ATOM | 6424 | CB | PRO | B | 427 | −37.310 | 47.587 | 76.297 | 1.00 | 61.77 | B |
| ATOM | 6425 | CG | PRO | B | 427 | −37.717 | 46.630 | 77.308 | 1.00 | 61.66 | B |
| ATOM | 6426 | C | PRO | B | 427 | −35.171 | 48.806 | 75.729 | 1.00 | 63.26 | B |
| ATOM | 6427 | O | PRO | B | 427 | −35.236 | 48.845 | 74.503 | 1.00 | 65.82 | B |
| ATOM | 6428 | N | THR | B | 428 | −34.589 | 49.779 | 76.422 | 1.00 | 59.38 | B |
| ATOM | 6429 | CA | THR | B | 428 | −34.014 | 50.918 | 75.702 | 1.00 | 57.40 | B |
| ATOM | 6430 | CB | THR | B | 428 | −32.943 | 50.452 | 74.661 | 1.00 | 57.11 | B |
| ATOM | 6431 | OG1 | THR | B | 428 | −31.653 | 50.400 | 75.285 | 1.00 | 56.66 | B |
| ATOM | 6432 | CG2 | THR | B | 428 | −32.872 | 51.396 | 73.462 | 1.00 | 55.85 | B |
| ATOM | 6433 | C | THR | B | 428 | −35.079 | 51.754 | 74.972 | 1.00 | 57.49 | B |
| ATOM | 6434 | O | THR | B | 428 | −34.848 | 52.930 | 74.653 | 1.00 | 56.51 | B |
| ATOM | 6435 | N | TYR | B | 429 | −36.236 | 51.147 | 74.717 | 1.00 | 56.96 | B |
| ATOM | 6436 | CA | TYR | B | 429 | −37.352 | 51.808 | 74.032 | 1.00 | 57.37 | B |
| ATOM | 6437 | CB | TYR | B | 429 | −36.996 | 52.117 | 72.584 | 1.00 | 56.68 | B |
| ATOM | 6438 | CG | TYR | B | 429 | −38.129 | 52.753 | 71.826 | 1.00 | 57.55 | B |
| ATOM | 6439 | CD1 | TYR | B | 429 | −38.405 | 54.111 | 71.963 | 1.00 | 58.49 | B |
| ATOM | 6440 | CE1 | TYR | B | 429 | −39.451 | 54.714 | 71.263 | 1.00 | 57.70 | B |
| ATOM | 6441 | CD2 | TYR | B | 429 | −38.932 | 52.004 | 70.972 | 1.00 | 58.52 | B |
| ATOM | 6442 | CE2 | TYR | B | 429 | −39.984 | 52.597 | 70.264 | 1.00 | 58.90 | B |
| ATOM | 6443 | CZ | TYR | B | 429 | −40.234 | 53.953 | 70.415 | 1.00 | 58.28 | B |
| ATOM | 6444 | OH | TYR | B | 429 | −41.247 | 54.542 | 69.700 | 1.00 | 57.26 | B |
| ATOM | 6445 | C | TYR | B | 429 | −38.559 | 50.886 | 74.023 | 1.00 | 57.69 | B |
| ATOM | 6446 | O | TYR | B | 429 | −38.407 | 49.680 | 73.837 | 1.00 | 59.02 | B |
| ATOM | 6447 | N | VAL | B | 430 | −39.755 | 51.437 | 74.210 | 1.00 | 56.49 | B |
| ATOM | 6448 | CA | VAL | B | 430 | −40.957 | 50.607 | 74.204 | 1.00 | 55.77 | B |
| ATOM | 6449 | CB | VAL | B | 430 | −41.337 | 50.138 | 75.615 | 1.00 | 54.06 | B |
| ATOM | 6450 | CG1 | VAL | B | 430 | −42.438 | 49.105 | 75.540 | 1.00 | 53.82 | B |
| ATOM | 6451 | CG2 | VAL | B | 430 | −40.146 | 49.551 | 76.296 | 1.00 | 55.44 | B |
| ATOM | 6452 | C | VAL | B | 430 | −42.160 | 51.325 | 73.625 | 1.00 | 56.50 | B |
| ATOM | 6453 | O | VAL | B | 430 | −42.371 | 52.500 | 73.872 | 1.00 | 57.93 | B |
| ATOM | 6454 | N | ASN | B | 431 | −42.954 | 50.606 | 72.846 | 1.00 | 57.00 | B |
| ATOM | 6455 | CA | ASN | B | 431 | −44.153 | 51.162 | 72.238 | 1.00 | 56.14 | B |
| ATOM | 6456 | CB | ASN | B | 431 | −43.820 | 51.867 | 70.924 | 1.00 | 56.67 | B |
| ATOM | 6457 | CG | ASN | B | 431 | −44.735 | 53.050 | 70.642 | 1.00 | 56.99 | B |
| ATOM | 6458 | OD1 | ASN | B | 431 | −45.923 | 53.016 | 70.932 | 1.00 | 57.98 | B |
| ATOM | 6459 | ND2 | ASN | B | 431 | −44.178 | 54.099 | 70.056 | 1.00 | 58.22 | B |
| ATOM | 6460 | C | ASN | B | 431 | −45.046 | 49.965 | 71.959 | 1.00 | 56.42 | B |
| ATOM | 6461 | O | ASN | B | 431 | −44.594 | 48.815 | 72.009 | 1.00 | 57.70 | B |
| ATOM | 6462 | N | HIS | B | 432 | −46.316 | 50.217 | 71.674 | 1.00 | 54.88 | B |
| ATOM | 6463 | CA | HIS | B | 432 | −47.213 | 49.113 | 71.387 | 1.00 | 53.36 | B |
| ATOM | 6464 | CB | HIS | B | 432 | −47.400 | 48.230 | 72.622 | 1.00 | 54.82 | B |
| ATOM | 6465 | CG | HIS | B | 432 | −48.424 | 48.751 | 73.585 | 1.00 | 55.02 | B |
| ATOM | 6466 | CD2 | HIS | B | 432 | −49.603 | 48.231 | 73.999 | 1.00 | 53.39 | B |
| ATOM | 6467 | ND1 | HIS | B | 432 | −48.289 | 49.958 | 74.235 | 1.00 | 56.85 | B |
| ATOM | 6468 | CE1 | HIS | B | 432 | −49.339 | 50.156 | 75.010 | 1.00 | 55.34 | B |
| ATOM | 6469 | NE2 | HIS | B | 432 | −50.151 | 49.123 | 74.885 | 1.00 | 52.11 | B |
| ATOM | 6470 | C | HIS | B | 432 | −48.564 | 49.608 | 70.956 | 1.00 | 51.28 | B |
| ATOM | 6471 | O | HIS | B | 432 | −48.924 | 50.756 | 71.201 | 1.00 | 51.73 | B |
| ATOM | 6472 | N | LEU | B | 433 | −49.308 | 48.721 | 70.311 | 1.00 | 49.37 | B |
| ATOM | 6473 | CA | LEU | B | 433 | −50.652 | 49.023 | 69.877 | 1.00 | 47.54 | B |
| ATOM | 6474 | CB | LEU | B | 433 | −50.716 | 49.125 | 68.368 | 1.00 | 47.20 | B |
| ATOM | 6475 | CG | LEU | B | 433 | −52.062 | 49.639 | 67.859 | 1.00 | 47.48 | B |
| ATOM | 6476 | CD1 | LEU | B | 433 | −52.237 | 51.064 | 68.334 | 1.00 | 45.37 | B |
| ATOM | 6477 | CD2 | LEU | B | 433 | −52.129 | 49.578 | 66.333 | 1.00 | 46.68 | B |
| ATOM | 6478 | C | LEU | B | 433 | −51.506 | 47.865 | 70.361 | 1.00 | 47.34 | B |
| ATOM | 6479 | O | LEU | B | 433 | −51.068 | 46.723 | 70.329 | 1.00 | 47.30 | B |
| ATOM | 6480 | N | TYR | B | 434 | −52.712 | 48.157 | 70.832 | 1.00 | 47.68 | B |
| ATOM | 6481 | CA | TYR | B | 434 | −53.606 | 47.122 | 71.330 | 1.00 | 48.15 | B |
| ATOM | 6482 | CB | TYR | B | 434 | −54.091 | 47.468 | 72.725 | 1.00 | 49.63 | B |
| ATOM | 6483 | CG | TYR | B | 434 | −55.324 | 46.690 | 73.153 | 1.00 | 54.79 | B |
| ATOM | 6484 | CD1 | TYR | B | 434 | −55.305 | 45.290 | 73.230 | 1.00 | 55.18 | B |
| ATOM | 6485 | CE1 | TYR | B | 434 | −56.422 | 44.578 | 73.684 | 1.00 | 55.22 | B |
| ATOM | 6486 | CD2 | TYR | B | 434 | −56.501 | 47.357 | 73.535 | 1.00 | 54.94 | B |
| ATOM | 6487 | CE2 | TYR | B | 434 | −57.619 | 46.652 | 73.990 | 1.00 | 54.83 | B |
| ATOM | 6488 | CZ | TYR | B | 434 | −57.573 | 45.264 | 74.063 | 1.00 | 55.37 | B |
| ATOM | 6489 | OH | TYR | B | 434 | −58.672 | 44.561 | 74.509 | 1.00 | 56.15 | B |
| ATOM | 6490 | C | TYR | B | 434 | −54.820 | 46.892 | 70.454 | 1.00 | 47.96 | B |
| ATOM | 6491 | O | TYR | B | 434 | −55.634 | 47.779 | 70.270 | 1.00 | 46.36 | B |
| ATOM | 6492 | N | TRP | B | 435 | −54.940 | 45.688 | 69.915 | 1.00 | 49.49 | B |
| ATOM | 6493 | CA | TRP | B | 435 | −56.088 | 45.358 | 69.087 | 1.00 | 50.44 | B |
| ATOM | 6494 | CB | TRP | B | 435 | −55.676 | 44.544 | 67.850 | 1.00 | 48.08 | B |
| ATOM | 6495 | CG | TRP | B | 435 | −54.606 | 45.137 | 67.012 | 1.00 | 45.28 | B |
| ATOM | 6496 | CD2 | TRP | B | 435 | −54.783 | 45.958 | 65.856 | 1.00 | 45.50 | B |
| ATOM | 6497 | CE2 | TRP | B | 435 | −53.492 | 46.289 | 65.374 | 1.00 | 45.32 | B |
| ATOM | 6498 | CE3 | TRP | B | 435 | −55.906 | 46.452 | 65.177 | 1.00 | 43.24 | B |
| ATOM | 6499 | CD1 | TRP | B | 435 | −53.259 | 45.004 | 67.186 | 1.00 | 46.11 | B |
| ATOM | 6500 | NE1 | TRP | B | 435 | −52.579 | 45.694 | 66.204 | 1.00 | 45.36 | B |
| ATOM | 6501 | CZ2 | TRP | B | 435 | −53.298 | 47.089 | 64.245 | 1.00 | 43.21 | B |
| ATOM | 6502 | CZ3 | TRP | B | 435 | −55.713 | 47.249 | 64.053 | 1.00 | 40.69 | B |
| ATOM | 6503 | CH2 | TRP | B | 435 | −54.418 | 47.559 | 63.599 | 1.00 | 42.79 | B |
| ATOM | 6504 | C | TRP | B | 435 | −57.013 | 44.511 | 69.971 | 1.00 | 51.61 | B |
| ATOM | 6505 | O | TRP | B | 435 | −56.610 | 43.454 | 70.465 | 1.00 | 51.44 | B |
| ATOM | 6506 | N | SER | B | 436 | −58.242 | 44.975 | 70.184 | 1.00 | 52.65 | B |
| ATOM | 6507 | CA | SER | B | 436 | −59.180 | 44.216 | 71.006 | 1.00 | 53.15 | B |
| ATOM | 6508 | CB | SER | B | 436 | −60.274 | 45.125 | 71.570 | 1.00 | 53.47 | B |
| ATOM | 6509 | OG | SER | B | 436 | −61.101 | 45.636 | 70.540 | 1.00 | 56.72 | B |
| ATOM | 6510 | C | SER | B | 436 | −59.805 | 43.095 | 70.178 | 1.00 | 53.17 | B |
| ATOM | 6511 | O | SER | B | 436 | −60.337 | 42.134 | 70.726 | 1.00 | 53.26 | B |
| ATOM | 6512 | N | LYS | B | 437 | −59.733 | 43.219 | 68.857 | 1.00 | 52.53 | B |
| ATOM | 6513 | CA | LYS | B | 437 | −60.279 | 42.198 | 67.979 | 1.00 | 53.45 | B |
| ATOM | 6514 | CB | LYS | B | 437 | −61.655 | 42.607 | 67.464 | 1.00 | 53.80 | B |
| ATOM | 6515 | CG | LYS | B | 437 | −62.658 | 42.770 | 68.567 | 1.00 | 54.76 | B |
| ATOM | 6516 | CD | LYS | B | 437 | −64.050 | 42.962 | 68.024 | 1.00 | 57.22 | B |
| ATOM | 6517 | CE | LYS | B | 437 | −65.009 | 43.264 | 69.174 | 1.00 | 60.87 | B |
| ATOM | 6518 | NZ | LYS | B | 437 | −64.629 | 44.502 | 69.939 | 1.00 | 62.95 | B |
| ATOM | 6519 | C | LYS | B | 437 | −59.340 | 41.969 | 66.818 | 1.00 | 53.82 | B |
| ATOM | 6520 | O | LYS | B | 437 | −59.671 | 42.239 | 65.664 | 1.00 | 56.00 | B |
| ATOM | 6521 | N | ALA | B | 438 | −58.167 | 41.448 | 67.138 | 1.00 | 54.07 | B |
| ATOM | 6522 | CA | ALA | B | 438 | −57.141 | 41.191 | 66.149 | 1.00 | 53.78 | B |
| ATOM | 6523 | CB | ALA | B | 438 | −55.902 | 40.664 | 66.841 | 1.00 | 54.25 | B |
| ATOM | 6524 | C | ALA | B | 438 | −57.572 | 40.242 | 65.037 | 1.00 | 53.51 | B |
| ATOM | 6525 | O | ALA | B | 438 | −57.503 | 40.588 | 63.852 | 1.00 | 51.18 | B |
| ATOM | 6526 | N | PHE | B | 439 | −58.013 | 39.045 | 65.410 | 1.00 | 53.40 | B |
| ATOM | 6527 | CA | PHE | B | 439 | −58.418 | 38.082 | 64.397 | 1.00 | 54.29 | B |
| ATOM | 6528 | CB | PHE | B | 439 | −57.428 | 36.927 | 64.286 | 1.00 | 51.25 | B |
| ATOM | 6529 | CG | PHE | B | 439 | −56.040 | 37.320 | 63.926 | 1.00 | 49.16 | B |
| ATOM | 6530 | CD1 | PHE | B | 439 | −55.138 | 37.730 | 64.911 | 1.00 | 49.16 | B |
| ATOM | 6531 | CD2 | PHE | B | 439 | −55.582 | 37.148 | 62.626 | 1.00 | 46.99 | B |
| ATOM | 6532 | CE1 | PHE | B | 439 | −53.806 | 37.943 | 64.607 | 1.00 | 45.30 | B |
| ATOM | 6533 | CE2 | PHE | B | 439 | −54.260 | 37.357 | 62.315 | 1.00 | 44.20 | B |
| ATOM | 6534 | CZ | PHE | B | 439 | −53.370 | 37.751 | 63.310 | 1.00 | 45.16 | B |
| ATOM | 6535 | C | PHE | B | 439 | −59.765 | 37.448 | 64.677 | 1.00 | 56.70 | B |
| ATOM | 6536 | O | PHE | B | 439 | −60.155 | 37.263 | 65.832 | 1.00 | 55.87 | B |
| ATOM | 6537 | N | PRO | B | 440 | −60.476 | 37.068 | 63.607 | 1.00 | 58.91 | B |
| ATOM | 6538 | CD | PRO | B | 440 | −60.068 | 37.300 | 62.218 | 1.00 | 59.37 | B |
| ATOM | 6539 | CA | PRO | B | 440 | −61.789 | 36.427 | 63.649 | 1.00 | 61.80 | B |
| ATOM | 6540 | CB | PRO | B | 440 | −62.214 | 36.400 | 62.187 | 1.00 | 61.04 | B |
| ATOM | 6541 | CG | PRO | B | 440 | −61.386 | 37.473 | 61.560 | 1.00 | 61.49 | B |
| ATOM | 6542 | C | PRO | B | 440 | −61.534 | 35.016 | 64.141 | 1.00 | 64.75 | B |
| ATOM | 6543 | O | PRO | B | 440 | −60.661 | 34.319 | 63.615 | 1.00 | 65.71 | B |
| ATOM | 6544 | N | LEU | B | 441 | −62.284 | 34.579 | 65.135 | 1.00 | 67.20 | B |
| ATOM | 6545 | CA | LEU | B | 441 | −62.072 | 33.242 | 65.629 | 1.00 | 70.18 | B |
| ATOM | 6546 | CB | LEU | B | 441 | −62.273 | 33.216 | 67.144 | 1.00 | 70.20 | B |
| ATOM | 6547 | CG | LEU | B | 441 | −61.301 | 32.319 | 67.920 | 1.00 | 71.16 | B |
| ATOM | 6548 | CD1 | LEU | B | 441 | −59.905 | 32.437 | 67.311 | 1.00 | 69.66 | B |
| ATOM | 6549 | CD2 | LEU | B | 441 | −61.296 | 32.715 | 69.407 | 1.00 | 70.76 | B |
| ATOM | 6550 | C | LEU | B | 441 | −63.010 | 32.263 | 64.930 | 1.00 | 73.11 | B |
| ATOM | 6551 | O | LEU | B | 441 | −64.212 | 32.234 | 65.212 | 1.00 | 72.91 | B |
| ATOM | 6552 | N | TYR | B | 442 | −62.449 | 31.495 | 63.989 | 1.00 | 76.53 | B |
| ATOM | 6553 | CA | TYR | B | 442 | −63.190 | 30.472 | 63.246 | 1.00 | 79.51 | B |
| ATOM | 6554 | CB | TYR | B | 442 | −62.338 | 29.884 | 62.116 | 1.00 | 80.74 | B |
| ATOM | 6555 | CG | TYR | B | 442 | −62.008 | 30.804 | 60.951 | 1.00 | 82.42 | B |
| ATOM | 6556 | CD1 | TYR | B | 442 | −61.258 | 30.329 | 59.862 | 1.00 | 82.35 | B |
| ATOM | 6557 | CE1 | TYR | B | 442 | −60.970 | 31.142 | 58.765 | 1.00 | 81.32 | B |
| ATOM | 6558 | CD2 | TYR | B | 442 | −62.460 | 32.123 | 60.911 | 1.00 | 82.09 | B |
| ATOM | 6559 | CE2 | TYR | B | 442 | −62.177 | 32.946 | 59.812 | 1.00 | 82.04 | B |
| ATOM | 6560 | CZ | TYR | B | 442 | −61.433 | 32.444 | 58.744 | 1.00 | 81.67 | B |
| ATOM | 6561 | OH | TYR | B | 442 | −61.161 | 33.235 | 57.650 | 1.00 | 82.39 | B |
| ATOM | 6562 | C | TYR | B | 442 | −63.487 | 29.348 | 64.236 | 1.00 | 81.16 | B |
| ATOM | 6563 | O | TYR | B | 442 | −62.778 | 28.347 | 64.263 | 1.00 | 81.25 | B |
| ATOM | 6564 | N | GLY | B | 443 | −64.528 | 29.515 | 65.045 | 1.00 | 83.37 | B |
| ATOM | 6565 | CA | GLY | B | 443 | −64.863 | 28.505 | 66.033 | 1.00 | 85.66 | B |
| ATOM | 6566 | C | GLY | B | 443 | −65.752 | 27.365 | 65.567 | 1.00 | 87.55 | B |
| ATOM | 6567 | O | GLY | B | 443 | −65.720 | 26.959 | 64.401 | 1.00 | 86.71 | B |
| ATOM | 6568 | N | ALA | B | 444 | −66.549 | 26.850 | 66.499 | 1.00 | 89.34 | B |
| ATOM | 6569 | CA | ALA | B | 444 | −67.459 | 25.742 | 66.230 | 1.00 | 91.57 | B |
| ATOM | 6570 | CB | ALA | B | 444 | −68.072 | 25.252 | 67.544 | 1.00 | 91.20 | B |
| ATOM | 6571 | C | ALA | B | 444 | −68.566 | 26.117 | 65.241 | 1.00 | 92.90 | B |
| ATOM | 6572 | O | ALA | B | 444 | −68.654 | 25.563 | 64.140 | 1.00 | 93.28 | B |
| ATOM | 6573 | N | ASN | B | 445 | −69.409 | 27.062 | 65.641 | 1.00 | 94.54 | B |
| ATOM | 6574 | CA | ASN | B | 445 | −70.523 | 27.506 | 64.802 | 1.00 | 95.57 | B |
| ATOM | 6575 | CB | ASN | B | 445 | −71.510 | 28.331 | 65.635 | 1.00 | 97.22 | B |
| ATOM | 6576 | CG | ASN | B | 445 | −70.915 | 29.660 | 66.107 | 1.00 | 99.36 | B |
| ATOM | 6577 | OD1 | ASN | B | 445 | −71.573 | 30.429 | 66.812 | 1.00 | 100.00 | B |
| ATOM | 6578 | ND2 | ASN | B | 445 | −69.664 | 29.933 | 65.718 | 1.00 | 99.63 | B |
| ATOM | 6579 | C | ASN | B | 445 | −70.083 | 28.338 | 63.602 | 1.00 | 94.62 | B |
| ATOM | 6580 | O | ASN | B | 445 | −70.846 | 29.175 | 63.122 | 1.00 | 93.34 | B |
| ATOM | 6581 | N | TYR | B | 446 | −68.864 | 28.129 | 63.120 | 1.00 | 94.45 | B |
| ATOM | 6582 | CA | TYR | B | 446 | −68.412 | 28.908 | 61.978 | 1.00 | 94.94 | B |
| ATOM | 6583 | CB | TYR | B | 446 | −66.882 | 28.980 | 61.927 | 1.00 | 96.55 | B |
| ATOM | 6584 | CG | TYR | B | 446 | −66.407 | 30.389 | 61.654 | 1.00 | 97.28 | B |
| ATOM | 6585 | CD1 | TYR | B | 446 | −66.578 | 31.396 | 62.612 | 1.00 | 97.62 | B |
| ATOM | 6586 | CE1 | TYR | B | 446 | −66.246 | 32.720 | 62.338 | 1.00 | 98.41 | B |
| ATOM | 6587 | CD2 | TYR | B | 446 | −65.880 | 30.741 | 60.411 | 1.00 | 97.38 | B |
| ATOM | 6588 | CE2 | TYR | B | 446 | −65.545 | 32.063 | 60.123 | 1.00 | 98.52 | B |
| ATOM | 6589 | CZ | TYR | B | 446 | −65.731 | 33.049 | 61.089 | 1.00 | 99.08 | B |
| ATOM | 6590 | OH | TYR | B | 446 | −65.413 | 34.360 | 60.803 | 1.00 | 98.78 | B |
| ATOM | 6591 | C | TYR | B | 446 | −68.965 | 28.353 | 60.671 | 1.00 | 94.24 | B |
| ATOM | 6592 | O | TYR | B | 446 | −68.858 | 28.985 | 59.616 | 1.00 | 93.63 | B |
| ATOM | 6593 | N | ASP | B | 447 | −69.563 | 27.169 | 60.756 | 1.00 | 94.15 | B |
| ATOM | 6594 | CA | ASP | B | 447 | −70.168 | 26.519 | 59.601 | 1.00 | 93.15 | B |
| ATOM | 6595 | CB | ASP | B | 447 | −70.499 | 25.062 | 59.925 | .00 | 93.37 | B |
| ATOM | 6596 | CG | ASP | B | 447 | −69.300 | 24.289 | 60.432 | .00 | 93.42 | B |
| ATOM | 6597 | OD1 | ASP | B | 447 | −69.476 | 23.124 | 60.844 | .00 | 93.55 | B |
| ATOM | 6598 | OD2 | ASP | B | 447 | −68.181 | 24.845 | 60.418 | .00 | 93.56 | B |
| ATOM | 6599 | C | ASP | B | 447 | −71.456 | 27.271 | 59.302 | 1.00 | 92.79 | B |
| ATOM | 6600 | O | ASP | B | 447 | −71.899 | 27.339 | 58.156 | 1.00 | 92.78 | B |
| ATOM | 6601 | N | SER | B | 448 | −72.049 | 27.837 | 60.352 | 1.00 | 92.46 | B |
| ATOM | 6602 | CA | SER | B | 448 | −73.290 | 28.595 | 60.231 | 1.00 | 92.63 | B |
| ATOM | 6603 | CB | SER | B | 448 | −74.096 | 28.506 | 61.532 | 1.00 | 92.05 | B |
| ATOM | 6604 | OG | SER | B | 448 | −73.556 | 29.354 | 62.534 | 1.00 | 92.63 | B |
| ATOM | 6605 | C | SER | B | 448 | −73.033 | 30.069 | 59.891 | 1.00 | 93.28 | B |
| ATOM | 6606 | O | SER | B | 448 | −73.836 | 30.696 | 59.192 | 1.00 | 94.49 | B |
| ATOM | 6607 | N | VAL | B | 449 | −71.925 | 30.623 | 60.390 | 1.00 | 92.87 | B |
| ATOM | 6608 | CA | VAL | B | 449 | −71.576 | 32.019 | 60.123 | 1.00 | 91.33 | B |
| ATOM | 6609 | CB | VAL | B | 449 | −70.174 | 32.368 | 60.676 | .00 | 91.03 | B |
| ATOM | 6610 | CG1 | VAL | B | 449 | −69.826 | 33.811 | 60.343 | .00 | 90.60 | B |
| ATOM | 6611 | CG2 | VAL | B | 449 | −70.140 | 32.154 | 62.180 | .00 | 90.56 | B |
| ATOM | 6612 | C | VAL | B | 449 | −71.587 | 32.247 | 58.612 | 1.00 | 91.21 | B |
| ATOM | 6613 | O | VAL | B | 449 | −72.273 | 33.143 | 58.115 | 1.00 | 90.75 | B |
| ATOM | 6614 | N | LEU | B | 450 | −70.837 | 31.422 | 57.884 | 1.00 | 90.51 | B |
| ATOM | 6615 | CA | LEU | B | 450 | −70.768 | 31.528 | 56.427 | 1.00 | 90.68 | B |
| ATOM | 6616 | CB | LEU | B | 450 | −69.953 | 30.372 | 55.856 | 1.00 | 91.28 | B |
| ATOM | 6617 | CG | LEU | B | 450 | −68.524 | 30.239 | 56.380 | 1.00 | 92.05 | B |
| ATOM | 6618 | CD1 | LEU | B | 450 | −67.938 | 28.912 | 55.924 | 1.00 | 92.19 | B |
| ATOM | 6619 | CD2 | LEU | B | 450 | −67.685 | 31.412 | 55.886 | 1.00 | 91.35 | B |
| ATOM | 6620 | C | LEU | B | 450 | −72.155 | 31.528 | 55.786 | 1.00 | 90.61 | B |
| ATOM | 6621 | O | LEU | B | 450 | −72.378 | 32.209 | 54.783 | 1.00 | 90.57 | B |
| ATOM | 6622 | N | ASP | B | 451 | −73.076 | 30.751 | 56.365 | 1.00 | 90.31 | B |
| ATOM | 6623 | CA | ASP | B | 451 | −74.455 | 30.654 | 55.876 | 1.00 | 88.94 | B |
| ATOM | 6624 | CB | ASP | B | 451 | −75.255 | 29.651 | 56.714 | .00 | 88.76 | B |
| ATOM | 6625 | CG | ASP | B | 451 | −74.782 | 28.225 | 56.534 | .00 | 88.63 | B |
| ATOM | 6626 | OD1 | ASP | B | 451 | −74.857 | 27.713 | 55.398 | .00 | 88.61 | B |
| ATOM | 6627 | OD2 | ASP | B | 451 | −74.339 | 27.616 | 57.529 | .00 | 88.65 | B |
| ATOM | 6628 | C | ASP | B | 451 | −75.159 | 32.007 | 55.939 | 1.00 | 88.12 | B |
| ATOM | 6629 | O | ASP | B | 451 | −75.881 | 32.378 | 55.012 | 1.00 | 87.87 | B |
| ATOM | 6630 | N | ALA | B | 452 | −74.951 | 32.730 | 57.043 | 1.00 | 86.85 | B |
| ATOM | 6631 | CA | ALA | B | 452 | −75.565 | 34.042 | 57.244 | 1.00 | 86.11 | B |
| ATOM | 6632 | CB | ALA | B | 452 | −75.311 | 34.530 | 58.675 | 1.00 | 85.79 | B |
| ATOM | 6633 | C | ALA | B | 452 | −75.028 | 35.059 | 56.239 | 1.00 | 85.76 | B |
| ATOM | 6634 | O | ALA | B | 452 | −75.792 | 35.839 | 55.660 | 1.00 | 84.98 | B |
| ATOM | 6635 | N | ILE | B | 453 | −73.710 | 35.046 | 56.037 | 1.00 | 85.41 | B |
| ATOM | 6636 | CA | ILE | B | 453 | −73.069 | 35.964 | 55.096 | 1.00 | 84.36 | B |
| ATOM | 6637 | CB | ILE | B | 453 | −71.547 | 35.720 | 55.006 | 1.00 | 82.97 | B |
| ATOM | 6638 | CG2 | ILE | B | 453 | −70.934 | 36.639 | 53.960 | .00 | 82.67 | B |
| ATOM | 6639 | CG1 | ILE | B | 453 | −70.907 | 35.961 | 56.374 | .00 | 82.23 | B |
| ATOM | 6640 | CD1 | ILE | B | 453 | −69.420 | 35.722 | 56.403 | 1.00 | 80.81 | B |
| ATOM | 6641 | C | ILE | B | 453 | −73.683 | 35.757 | 53.721 | 1.00 | 84.23 | B |
| ATOM | 6642 | O | ILE | B | 453 | −73.890 | 36.709 | 52.963 | 1.00 | 83.77 | B |
| ATOM | 6643 | N | ASP | B | 454 | −73.978 | 34.502 | 53.408 | 1.00 | 84.23 | B |
| ATOM | 6644 | CA | ASP | B | 454 | −74.588 | 34.172 | 52.131 | 1.00 | 84.90 | B |
| ATOM | 6645 | CB | ASP | B | 454 | −74.398 | 32.684 | 51.827 | 1.00 | 85.31 | B |
| ATOM | 6646 | CG | ASP | B | 454 | −72.926 | 32.303 | 51.688 | 1.00 | 86.55 | B |
| ATOM | 6647 | OD1 | ASP | B | 454 | −72.223 | 32.937 | 50.863 | 1.00 | 86.06 | B |
| ATOM | 6648 | OD2 | ASP | B | 454 | −72.469 | 31.374 | 52.397 | 1.00 | 86.66 | B |
| ATOM | 6649 | C | ASP | B | 454 | −76.071 | 34.530 | 52.154 | 1.00 | 85.09 | B |
| ATOM | 6650 | O | ASP | B | 454 | −76.607 | 35.018 | 51.157 | 1.00 | 84.56 | B |
| ATOM | 6651 | N | LYS | B | 455 | −76.725 | 34.304 | 53.293 | 1.00 | 84.86 | B |
| ATOM | 6652 | CA | LYS | B | 455 | −78.141 | 34.620 | 53.414 | 1.00 | 85.12 | B |
| ATOM | 6653 | CB | LYS | B | 455 | −78.670 | 34.234 | 54.803 | .00 | 83.96 | B |
| ATOM | 6654 | CG | LYS | B | 455 | −80.165 | 34.485 | 54.995 | .00 | 82.85 | B |
| ATOM | 6655 | CD | LYS | B | 455 | −80.656 | 34.013 | 56.361 | .00 | 82.02 | B |
| ATOM | 6656 | CE | LYS | B | 455 | −80.530 | 32.503 | 56.514 | .00 | 81.55 | B |
| ATOM | 6657 | NZ | LYS | B | 455 | −81.336 | 31.770 | 55.498 | .00 | 81.16 | B |
| ATOM | 6658 | C | LYS | B | 455 | −78.306 | 36.113 | 53.188 | 1.00 | 85.87 | B |
| ATOM | 6659 | O | LYS | B | 455 | −79.200 | 36.551 | 52.462 | 1.00 | 85.52 | B |
| ATOM | 6660 | N | MSE | B | 456 | −77.418 | 36.887 | 53.802 | 1.00 | 87.48 | B |
| ATOM | 6661 | CA | MSE | B | 456 | −77.461 | 38.337 | 53.685 | 1.00 | 89.96 | B |
| ATOM | 6662 | CB | MSE | B | 456 | −76.385 | 38.969 | 54.583 | 1.00 | 91.72 | B |
| ATOM | 6663 | CG | MSE | B | 456 | −76.443 | 40.499 | 54.649 | 1.00 | 95.00 | B |
| ATOM | 6664 | SE | MSE | B | 456 | −75.027 | 41.363 | 55.735 | 1.00 | 100.00 | B |
| ATOM | 6665 | CE | MSE | B | 456 | −76.092 | 41.938 | 57.268 | 1.00 | 96.22 | B |
| ATOM | 6666 | C | MSE | B | 456 | −77.262 | 38.752 | 52.228 | 1.00 | 89.91 | B |
| ATOM | 6667 | O | MSE | B | 456 | −77.996 | 39.598 | 51.712 | 1.00 | 89.51 | B |
| ATOM | 6668 | N | GLU | B | 457 | −76.281 | 38.137 | 51.567 | 1.00 | 90.38 | B |
| ATOM | 6669 | CA | GLU | B | 457 | −75.973 | 38.434 | 50.162 | 1.00 | 91.54 | B |
| ATOM | 6670 | CB | GLU | B | 457 | −74.641 | 37.796 | 49.761 | 1.00 | 90.76 | B |
| ATOM | 6671 | CG | GLU | B | 457 | −73.428 | 38.402 | 50.436 | 1.00 | 91.36 | B |
| ATOM | 6672 | CD | GLU | B | 457 | −72.123 | 37.765 | 49.970 | 1.00 | 92.56 | B |
| ATOM | 6673 | OE1 | GLU | B | 457 | −71.907 | 36.561 | 50.245 | 1.00 | 92.80 | B |
| ATOM | 6674 | OE2 | GLU | B | 457 | −71.311 | 38.466 | 49.325 | 1.00 | 93.08 | B |
| ATOM | 6675 | C | GLU | B | 457 | −77.053 | 38.001 | 49.160 | 1.00 | 92.36 | B |
| ATOM | 6676 | O | GLU | B | 457 | −77.420 | 38.775 | 48.272 | 1.00 | 92.32 | B |
| ATOM | 6677 | N | LYS | B | 458 | −77.532 | 36.760 | 49.282 | 1.00 | 93.50 | B |
| ATOM | 6678 | CA | LYS | B | 458 | −78.581 | 36.240 | 48.401 | 1.00 | 93.22 | B |
| ATOM | 6679 | CB | LYS | B | 458 | −78.831 | 34.756 | 48.682 | .00 | 93.36 | B |
| ATOM | 6680 | CG | LYS | B | 458 | −77.650 | 33.854 | 48.363 | .00 | 93.28 | B |
| ATOM | 6681 | CD | LYS | B | 458 | −77.987 | 32.394 | 48.622 | .00 | 93.29 | B |
| ATOM | 6682 | CE | LYS | B | 458 | −76.828 | 31.483 | 48.250 | .00 | 93.28 | B |
| ATOM | 6683 | NZ | LYS | B | 458 | −75.598 | 31.797 | 49.027 | .00 | 93.28 | B |
| ATOM | 6684 | C | LYS | B | 458 | −79.861 | 37.037 | 48.655 | 1.00 | 93.68 | B |
| ATOM | 6685 | O | LYS | B | 458 | −80.726 | 37.153 | 47.783 | 1.00 | 93.36 | B |
| ATOM | 6686 | N | ASN | B | 459 | −79.967 | 37.579 | 49.866 | 1.00 | 93.77 | B |
| ATOM | 6687 | CA | ASN | B | 459 | −81.110 | 38.395 | 50.258 | 1.00 | 93.51 | B |
| ATOM | 6688 | CB | ASN | B | 459 | −81.181 | 38.502 | 51.795 | 1.00 | 93.10 | B |
| ATOM | 6689 | CG | ASN | B | 459 | −82.116 | 39.611 | 52.279 | 1.00 | 93.31 | B |
| ATOM | 6690 | OD1 | ASN | B | 459 | −83.279 | 39.684 | 51.893 | 1.00 | 93.47 | B |
| ATOM | 6691 | ND2 | ASN | B | 459 | −81.600 | 40.474 | 53.138 | 1.00 | 93.64 | B |
| ATOM | 6692 | C | ASN | B | 459 | −80.942 | 39.777 | 49.618 | 1.00 | 93.18 | B |
| ATOM | 6693 | O | ASN | B | 459 | −81.289 | 39.966 | 48.447 | 1.00 | 93.00 | B |
| ATOM | 6694 | N | LEU | B | 460 | −80.398 | 40.721 | 50.389 | 1.00 | 91.85 | B |
| ATOM | 6695 | CA | LEU | B | 460 | −80.164 | 42.093 | 49.945 | 1.00 | 90.17 | B |
| ATOM | 6696 | CB | LEU | B | 460 | −79.491 | 42.886 | 51.069 | 1.00 | 87.94 | B |
| ATOM | 6697 | CG | LEU | B | 460 | −80.378 | 43.722 | 51.997 | .00 | 87.77 | B |
| ATOM | 6698 | CD1 | LEU | B | 460 | −81.729 | 43.067 | 52.218 | .00 | 87.24 | B |
| ATOM | 6699 | CD2 | LEU | B | 460 | −79.649 | 43.916 | 53.307 | .00 | 87.24 | B |
| ATOM | 6700 | C | LEU | B | 460 | −79.299 | 42.128 | 48.688 | 1.00 | 90.09 | B |
| ATOM | 6701 | O | LEU | B | 460 | −78.079 | 42.009 | 48.763 | 1.00 | 89.40 | B |
| ATOM | 6702 | N | PRO | B | 461 | −79.932 | 42.293 | 47.510 | 1.00 | 90.57 | B |
| ATOM | 6703 | CD | PRO | B | 461 | −81.393 | 42.380 | 47.310 | 1.00 | 90.22 | B |
| ATOM | 6704 | CA | PRO | B | 461 | −79.232 | 42.342 | 46.217 | 1.00 | 89.83 | B |
| ATOM | 6705 | CB | PRO | B | 461 | −80.379 | 42.448 | 45.203 | 1.00 | 90.79 | B |
| ATOM | 6706 | CG | PRO | B | 461 | −81.549 | 41.797 | 45.924 | 1.00 | 90.50 | B |
| ATOM | 6707 | C | PRO | B | 461 | −78.254 | 43.506 | 46.094 | 1.00 | 88.26 | B |
| ATOM | 6708 | O | PRO | B | 461 | −78.478 | 44.583 | 46.649 | 1.00 | 87.06 | B |
| ATOM | 6709 | N | GLY | B | 462 | −77.171 | 43.279 | 45.358 | 1.00 | 87.63 | B |
| ATOM | 6710 | CA | GLY | B | 462 | −76.172 | 44.317 | 45.169 | 1.00 | 86.51 | B |
| ATOM | 6711 | C | GLY | B | 462 | −75.114 | 44.357 | 46.256 | 1.00 | 84.45 | B |
| ATOM | 6712 | O | GLY | B | 462 | −73.968 | 44.728 | 45.998 | 1.00 | 84.56 | B |
| ATOM | 6713 | N | LEU | B | 463 | −75.506 | 43.979 | 47.469 | 1.00 | 82.25 | B |
| ATOM | 6714 | CA | LEU | B | 463 | −74.606 | 43.964 | 48.612 | 1.00 | 79.71 | B |
| ATOM | 6715 | CB | LEU | B | 463 | −75.404 | 43.987 | 49.909 | 1.00 | 79.01 | B |
| ATOM | 6716 | CG | LEU | B | 463 | −74.542 | 43.987 | 51.166 | 1.00 | 78.94 | B |
| ATOM | 6717 | CD1 | LEU | B | 463 | −73.817 | 45.312 | 51.250 | 1.00 | 79.20 | B |
| ATOM | 6718 | CD2 | LEU | B | 463 | −75.405 | 43.781 | 52.399 | 1.00 | 79.76 | B |
| ATOM | 6719 | C | LEU | B | 463 | −73.727 | 42.724 | 48.606 | 1.00 | 78.89 | B |
| ATOM | 6720 | O | LEU | B | 463 | −74.232 | 41.606 | 48.600 | 1.00 | 78.46 | B |
| ATOM | 6721 | N | PHE | B | 464 | −72.412 | 42.927 | 48.606 | 1.00 | 77.88 | B |
| ATOM | 6722 | CA | PHE | B | 464 | −71.469 | 41.814 | 48.629 | 1.00 | 77.01 | B |
| ATOM | 6723 | CB | PHE | B | 464 | −70.661 | 41.744 | 47.338 | 1.00 | 76.13 | B |
| ATOM | 6724 | CG | PHE | B | 464 | −71.491 | 41.563 | 46.113 | 1.00 | 75.87 | B |
| ATOM | 6725 | CD1 | PHE | B | 464 | −72.158 | 42.648 | 45.547 | 1.00 | 77.07 | B |
| ATOM | 6726 | CD2 | PHE | B | 464 | −71.591 | 40.309 | 45.511 | 1.00 | 75.36 | B |
| ATOM | 6727 | CE1 | PHE | B | 464 | −72.916 | 42.493 | 44.391 | 1.00 | 77.93 | B |
| ATOM | 6728 | CE2 | PHE | B | 464 | −72.341 | 40.128 | 44.357 | 1.00 | 75.21 | B |
| ATOM | 6729 | CZ | PHE | B | 464 | −73.008 | 41.222 | 43.791 | 1.00 | 78.57 | B |
| ATOM | 6730 | C | PHE | B | 464 | −70.504 | 41.954 | 49.800 | 1.00 | 76.70 | B |
| ATOM | 6731 | O | PHE | B | 464 | −69.882 | 42.999 | 49.986 | 1.00 | 76.74 | B |
| ATOM | 6732 | N | TYR | B | 465 | −70.383 | 40.898 | 50.591 | 1.00 | 75.93 | B |
| ATOM | 6733 | CA | TYR | B | 465 | −69.480 | 40.920 | 51.723 | 1.00 | 75.88 | B |
| ATOM | 6734 | CB | TYR | B | 465 | −70.037 | 40.048 | 52.851 | 1.00 | 75.49 | B |
| ATOM | 6735 | CG | TYR | B | 465 | −69.252 | 40.099 | 54.147 | 1.00 | 75.72 | B |
| ATOM | 6736 | CD1 | TYR | B | 465 | −68.044 | 39.414 | 54.289 | 1.00 | 75.67 | B |
| ATOM | 6737 | CE1 | TYR | B | 465 | −67.314 | 39.469 | 55.485 | 1.00 | 77.01 | B |
| ATOM | 6738 | CD2 | TYR | B | 465 | −69.717 | 40.844 | 55.233 | 1.00 | 76.41 | B |
| ATOM | 6739 | CE2 | TYR | B | 465 | −68.996 | 40.911 | 56.433 | 1.00 | 77.37 | B |
| ATOM | 6740 | CZ | TYR | B | 465 | −67.795 | 40.224 | 56.555 | 1.00 | 77.60 | B |
| ATOM | 6741 | OH | TYR | B | 465 | −67.076 | 40.308 | 57.735 | 1.00 | 77.51 | B |
| ATOM | 6742 | C | TYR | B | 465 | −68.155 | 40.373 | 51.220 | 1.00 | 76.45 | B |
| ATOM | 6743 | O | TYR | B | 465 | −68.129 | 39.518 | 50.333 | 1.00 | 75.75 | B |
| ATOM | 6744 | N | ALA | B | 466 | −67.059 | 40.887 | 51.767 | 1.00 | 77.54 | B |
| ATOM | 6745 | CA | ALA | B | 466 | −65.727 | 40.442 | 51.377 | 1.00 | 79.63 | B |
| ATOM | 6746 | CB | ALA | B | 466 | −65.278 | 41.157 | 50.114 | 1.00 | 78.02 | B |
| ATOM | 6747 | C | ALA | B | 466 | −64.719 | 40.685 | 52.495 | 1.00 | 81.66 | B |
| ATOM | 6748 | O | ALA | B | 466 | −63.573 | 41.055 | 52.229 | 1.00 | 82.51 | B |
| ATOM | 6749 | N | GLY | B | 467 | −65.151 | 40.483 | 53.741 | 1.00 | 82.80 | B |
| ATOM | 6750 | CA | GLY | B | 467 | −64.264 | 40.671 | 54.877 | 1.00 | 84.32 | B |
| ATOM | 6751 | C | GLY | B | 467 | −63.159 | 39.625 | 54.891 | 1.00 | 85.72 | B |
| ATOM | 6752 | O | GLY | B | 467 | −62.649 | 39.239 | 53.835 | 1.00 | 85.85 | B |
| ATOM | 6753 | N | ASN | B | 468 | −62.769 | 39.176 | 56.083 | 1.00 | 86.58 | B |
| ATOM | 6754 | CA | ASN | B | 468 | −61.728 | 38.151 | 56.206 | 1.00 | 87.02 | B |
| ATOM | 6755 | CB | ASN | B | 468 | −60.432 | 38.746 | 56.776 | 1.00 | 85.37 | B |
| ATOM | 6756 | CG | ASN | B | 468 | −60.492 | 38.962 | 58.268 | 1.00 | 84.58 | B |
| ATOM | 6757 | OD1 | ASN | B | 468 | −61.515 | 39.386 | 58.808 | 1.00 | 83.88 | B |
| ATOM | 6758 | ND2 | ASN | B | 468 | −59.383 | 38.687 | 58.946 | 1.00 | 83.55 | B |
| ATOM | 6759 | C | ASN | B | 468 | −62.266 | 37.040 | 57.108 | 1.00 | 87.52 | B |
| ATOM | 6760 | O | ASN | B | 468 | −61.533 | 36.146 | 57.546 | 1.00 | 87.05 | B |
| ATOM | 6761 | N | HIS | B | 469 | −63.569 | 37.116 | 57.369 | 1.00 | 87.70 | B |
| ATOM | 6762 | CA | HIS | B | 469 | −64.257 | 36.132 | 58.184 | 1.00 | 87.49 | B |
| ATOM | 6763 | CB | HIS | B | 469 | −65.484 | 36.763 | 58.853 | 1.00 | 87.39 | B |
| ATOM | 6764 | CG | HIS | B | 469 | −65.151 | 37.884 | 59.791 | 1.00 | 87.20 | B |
| ATOM | 6765 | CD2 | HIS | B | 469 | −64.219 | 38.864 | 59.715 | 1.00 | 86.70 | B |
| ATOM | 6766 | ND1 | HIS | B | 469 | −65.820 | 38.083 | 60.981 | 1.00 | 86.68 | B |
| ATOM | 6767 | CE1 | HIS | B | 469 | −65.313 | 39.135 | 61.598 | 1.00 | 85.61 | B |
| ATOM | 6768 | NE2 | HIS | B | 469 | −64.340 | 39.628 | 60.852 | 1.00 | 86.59 | B |
| ATOM | 6769 | C | HIS | B | 469 | −64.676 | 34.990 | 57.263 | 1.00 | 87.13 | B |
| ATOM | 6770 | O | HIS | B | 469 | −65.224 | 33.980 | 57.706 | 1.00 | 86.43 | B |
| ATOM | 6771 | N | ARG | B | 470 | −64.395 | 35.159 | 55.975 | 1.00 | 87.02 | B |
| ATOM | 6772 | CA | ARG | B | 470 | −64.736 | 34.150 | 54.985 | 1.00 | 87.14 | B |
| ATOM | 6773 | CB | ARG | B | 470 | −65.575 | 34.781 | 53.864 | 1.00 | 87.05 | B |
| ATOM | 6774 | CG | ARG | B | 470 | −66.398 | 33.783 | 53.061 | 1.00 | 86.74 | B |
| ATOM | 6775 | CD | ARG | B | 470 | −66.458 | 34.175 | 51.589 | 1.00 | 88.16 | B |
| ATOM | 6776 | NE | ARG | B | 470 | −67.287 | 35.349 | 51.295 | 1.00 | 88.75 | B |
| ATOM | 6777 | CZ | ARG | B | 470 | −68.622 | 35.355 | 51.287 | 1.00 | 89.07 | B |
| ATOM | 6778 | NH1 | ARG | B | 470 | −69.306 | 34.252 | 51.568 | 1.00 | 88.68 | B |
| ATOM | 6779 | NH2 | ARG | B | 470 | −69.280 | 36.460 | 50.959 | 1.00 | 88.17 | B |
| ATOM | 6780 | C | ARG | B | 470 | −63.476 | 33.490 | 54.397 | 1.00 | 86.91 | B |
| ATOM | 6781 | O | ARG | B | 470 | −63.040 | 32.432 | 54.870 | 1.00 | 87.02 | B |
| ATOM | 6782 | N | GLY | B | 471 | −62.889 | 34.128 | 53.386 | 1.00 | 85.58 | B |
| ATOM | 6783 | CA | GLY | B | 471 | −61.709 | 33.586 | 52.720 | 1.00 | 84.91 | B |
| ATOM | 6784 | C | GLY | B | 471 | −60.512 | 33.083 | 53.524 | 1.00 | 83.73 | B |
| ATOM | 6785 | O | GLY | B | 471 | −59.825 | 32.154 | 53.101 | 1.00 | 83.13 | B |
| ATOM | 6786 | N | GLY | B | 472 | −60.250 | 33.687 | 54.677 | 1.00 | 82.78 | B |
| ATOM | 6787 | CA | GLY | B | 472 | −59.111 | 33.274 | 55.479 | 1.00 | 80.64 | B |
| ATOM | 6788 | C | GLY | B | 472 | −58.393 | 34.498 | 56.015 | 1.00 | 79.60 | B |
| ATOM | 6789 | O | GLY | B | 472 | −58.385 | 35.548 | 55.366 | 1.00 | 80.28 | B |
| ATOM | 6790 | N | LEU | B | 473 | −57.779 | 34.374 | 57.186 | 1.00 | 77.45 | B |
| ATOM | 6791 | CA | LEU | B | 473 | −57.097 | 35.512 | 57.790 | 1.00 | 75.77 | B |
| ATOM | 6792 | CB | LEU | B | 473 | −57.260 | 35.460 | 59.311 | 1.00 | 74.13 | B |
| ATOM | 6793 | CG | LEU | B | 473 | −57.401 | 34.081 | 59.947 | 1.00 | 73.24 | B |
| ATOM | 6794 | CD1 | LEU | B | 473 | −56.293 | 33.149 | 59.482 | 1.00 | 73.45 | B |
| ATOM | 6795 | CD2 | LEU | B | 473 | −57.370 | 34.246 | 61.452 | 1.00 | 73.86 | B |
| ATOM | 6796 | C | LEU | B | 473 | −55.624 | 35.706 | 57.433 | 1.00 | 74.66 | B |
| ATOM | 6797 | O | LEU | B | 473 | −54.735 | 35.237 | 58.137 | 1.00 | 76.38 | B |
| ATOM | 6798 | N | SER | B | 474 | −55.376 | 36.417 | 56.338 | 1.00 | 72.61 | B |
| ATOM | 6799 | CA | SER | B | 474 | −54.018 | 36.706 | 55.883 | 1.00 | 70.49 | B |
| ATOM | 6800 | CB | SER | B | 474 | −53.357 | 35.452 | 55.315 | 1.00 | 67.58 | B |
| ATOM | 6801 | OG | SER | B | 474 | −53.899 | 35.129 | 54.055 | 1.00 | 66.91 | B |
| ATOM | 6802 | C | SER | B | 474 | −54.102 | 37.784 | 54.804 | 1.00 | 70.01 | B |
| ATOM | 6803 | O | SER | B | 474 | −55.010 | 37.764 | 53.973 | 1.00 | 70.65 | B |
| ATOM | 6804 | N | VAL | B | 475 | −53.171 | 38.732 | 54.826 | 1.00 | 68.49 | B |
| ATOM | 6805 | CA | VAL | B | 475 | −53.174 | 39.807 | 53.843 | 1.00 | 67.33 | B |
| ATOM | 6806 | CB | VAL | B | 475 | −51.804 | 40.489 | 53.776 | 1.00 | 66.92 | B |
| ATOM | 6807 | CG1 | VAL | B | 475 | −51.890 | 41.735 | 52.907 | 1.00 | 66.15 | B |
| ATOM | 6808 | CG2 | VAL | B | 475 | −51.334 | 40.826 | 55.177 | 1.00 | 67.34 | B |
| ATOM | 6809 | C | VAL | B | 475 | −53.519 | 39.284 | 52.446 | 1.00 | 66.74 | B |
| ATOM | 6810 | O | VAL | B | 475 | −54.434 | 39.791 | 51.787 | 1.00 | 65.55 | B |
| ATOM | 6811 | N | GLY | B | 476 | −52.784 | 38.259 | 52.014 | 1.00 | 65.69 | B |
| ATOM | 6812 | CA | GLY | B | 476 | −53.003 | 37.673 | 50.705 | 1.00 | 64.61 | B |
| ATOM | 6813 | C | GLY | B | 476 | −54.441 | 37.287 | 50.416 | 1.00 | 64.52 | B |
| ATOM | 6814 | O | GLY | B | 476 | −54.987 | 37.651 | 49.374 | 1.00 | 64.77 | B |
| ATOM | 6815 | N | LYS | B | 477 | −55.057 | 36.545 | 51.331 | 1.00 | 64.17 | B |
| ATOM | 6816 | CA | LYS | B | 477 | −56.437 | 36.109 | 51.159 | 1.00 | 63.53 | B |
| ATOM | 6817 | CB | LYS | B | 477 | −56.850 | 35.168 | 52.298 | 1.00 | 65.23 | B |
| ATOM | 6818 | CG | LYS | B | 477 | −56.293 | 33.743 | 52.183 | 1.00 | 67.80 | B |
| ATOM | 6819 | CD | LYS | B | 477 | −56.978 | 32.952 | 51.064 | 1.00 | 69.47 | B |
| ATOM | 6820 | CE | LYS | B | 477 | −56.487 | 31.505 | 50.984 | 1.00 | 69.01 | B |
| ATOM | 6821 | NZ | LYS | B | 477 | −55.069 | 31.403 | 50.518 | 1.00 | 68.19 | B |
| ATOM | 6822 | C | LYS | B | 477 | −57.415 | 37.269 | 51.076 | 1.00 | 62.50 | B |
| ATOM | 6823 | O | LYS | B | 477 | −58.125 | 37.405 | 50.087 | 1.00 | 62.53 | B |
| ATOM | 6824 | N | ALA | B | 478 | −57.459 | 38.102 | 52.109 | 1.00 | 62.43 | B |
| ATOM | 6825 | CA | ALA | B | 478 | −58.378 | 39.242 | 52.119 | 1.00 | 62.65 | B |
| ATOM | 6826 | CB | ALA | B | 478 | −58.141 | 40.100 | 53.358 | 1.00 | 63.60 | B |
| ATOM | 6827 | C | ALA | B | 478 | −58.220 | 40.092 | 50.862 | 1.00 | 62.26 | B |
| ATOM | 6828 | O | ALA | B | 478 | −59.184 | 40.655 | 50.345 | 1.00 | 62.20 | B |
| ATOM | 6829 | N | LEU | B | 479 | −56.994 | 40.192 | 50.376 | 1.00 | 62.27 | B |
| ATOM | 6830 | CA | LEU | B | 479 | −56.741 | 40.967 | 49.183 | 1.00 | 63.16 | B |
| ATOM | 6831 | CB | LEU | B | 479 | −55.249 | 40.952 | 48.881 | 1.00 | 62.49 | B |
| ATOM | 6832 | CG | LEU | B | 479 | −54.790 | 41.807 | 47.710 | 1.00 | 63.29 | B |
| ATOM | 6833 | CD1 | LEU | B | 479 | −53.320 | 42.149 | 47.910 | 1.00 | 63.65 | B |
| ATOM | 6834 | CD2 | LEU | B | 479 | −55.039 | 41.068 | 46.394 | 1.00 | 62.67 | B |
| ATOM | 6835 | C | LEU | B | 479 | −57.543 | 40.342 | 48.044 | 1.00 | 64.47 | B |
| ATOM | 6836 | O | LEU | B | 479 | −58.162 | 41.051 | 47.245 | 1.00 | 65.10 | B |
| ATOM | 6837 | N | SER | B | 480 | −57.532 | 39.008 | 47.988 | 1.00 | 65.73 | B |
| ATOM | 6838 | CA | SER | B | 480 | −58.261 | 38.256 | 46.970 | 1.00 | 65.34 | B |
| ATOM | 6839 | CB | SER | B | 480 | −57.911 | 36.772 | 47.040 | 1.00 | 63.28 | B |
| ATOM | 6840 | OG | SER | B | 480 | −56.676 | 36.521 | 46.416 | 1.00 | 64.58 | B |
| ATOM | 6841 | C | SER | B | 480 | −59.764 | 38.399 | 47.119 | 1.00 | 65.39 | B |
| ATOM | 6842 | O | SER | B | 480 | −60.474 | 38.583 | 46.134 | 1.00 | 65.83 | B |
| ATOM | 6843 | N | SER | B | 481 | −60.255 | 38.303 | 48.347 | 1.00 | 65.39 | B |
| ATOM | 6844 | CA | SER | B | 481 | −61.682 | 38.417 | 48.556 | 1.00 | 67.45 | B |
| ATOM | 6845 | CB | SER | B | 481 | −62.046 | 38.137 | 50.018 | 1.00 | 67.34 | B |
| ATOM | 6846 | OG | SER | B | 481 | −61.384 | 39.016 | 50.901 | 1.00 | 70.97 | B |
| ATOM | 6847 | C | SER | B | 481 | −62.141 | 39.798 | 48.138 | 1.00 | 68.30 | B |
| ATOM | 6848 | O | SER | B | 481 | −63.301 | 39.987 | 47.779 | 1.00 | 70.31 | B |
| ATOM | 6849 | N | GLY | B | 482 | −61.225 | 40.760 | 48.172 | 1.00 | 68.51 | B |
| ATOM | 6850 | CA | GLY | B | 482 | −61.566 | 42.111 | 47.765 | 1.00 | 70.06 | B |
| ATOM | 6851 | C | GLY | B | 482 | −61.720 | 42.188 | 46.255 | 1.00 | 70.65 | B |
| ATOM | 6852 | O | GLY | B | 482 | −62.693 | 42.742 | 45.746 | 1.00 | 70.10 | B |
| ATOM | 6853 | N | CYS | B | 483 | −60.747 | 41.628 | 45.542 | 1.00 | 71.87 | B |
| ATOM | 6854 | CA | CYS | B | 483 | −60.768 | 41.602 | 44.084 | 1.00 | 74.80 | B |
| ATOM | 6855 | CB | CYS | B | 483 | −59.523 | 40.889 | 43.550 | 1.00 | 76.13 | B |
| ATOM | 6856 | SG | CYS | B | 483 | −57.974 | 41.739 | 43.845 | 1.00 | 79.95 | B |
| ATOM | 6857 | C | CYS | B | 483 | −62.000 | 40.849 | 43.589 | 1.00 | 74.87 | B |
| ATOM | 6858 | O | CYS | B | 483 | −62.780 | 41.342 | 42.767 | 1.00 | 74.65 | B |
| ATOM | 6859 | N | ASN | B | 484 | −62.153 | 39.636 | 44.098 | 1.00 | 74.94 | B |
| ATOM | 6860 | CA | ASN | B | 484 | −63.257 | 38.792 | 43.721 | 1.00 | 75.18 | B |
| ATOM | 6861 | CB | ASN | B | 484 | −63.136 | 37.453 | 44.421 | 1.00 | 76.41 | B |
| ATOM | 6862 | CG | ASN | B | 484 | −63.878 | 36.376 | 43.699 | 1.00 | 79.19 | B |
| ATOM | 6863 | OD1 | ASN | B | 484 | −63.496 | 35.989 | 42.593 | 1.00 | 79.80 | B |
| ATOM | 6864 | ND2 | ASN | B | 484 | −64.961 | 35.887 | 44.303 | 1.00 | 81.77 | B |
| ATOM | 6865 | C | ASN | B | 484 | −64.600 | 39.433 | 44.057 | 1.00 | 75.64 | B |
| ATOM | 6866 | O | ASN | B | 484 | −65.621 | 39.079 | 43.468 | 1.00 | 76.56 | B |
| ATOM | 6867 | N | ALA | B | 485 | −64.609 | 40.366 | 45.005 | 1.00 | 74.45 | B |
| ATOM | 6868 | CA | ALA | B | 485 | −65.851 | 41.031 | 45.366 | 1.00 | 74.63 | B |
| ATOM | 6869 | CB | ALA | B | 485 | −65.727 | 41.683 | 46.730 | 1.00 | 73.36 | B |
| ATOM | 6870 | C | ALA | B | 485 | −66.145 | 42.082 | 44.303 | 1.00 | 75.36 | B |
| ATOM | 6871 | O | ALA | B | 485 | −67.291 | 42.263 | 43.881 | 1.00 | 75.77 | B |
| ATOM | 6872 | N | ALA | B | 486 | −65.092 | 42.761 | 43.863 | 1.00 | 75.72 | B |
| ATOM | 6873 | CA | ALA | B | 486 | −65.215 | 43.806 | 42.853 | 1.00 | 76.93 | B |
| ATOM | 6874 | CB | ALA | B | 486 | −63.843 | 44.425 | 42.572 | 1.00 | 76.30 | B |
| ATOM | 6875 | C | ALA | B | 486 | −65.855 | 43.325 | 41.542 | 1.00 | 77.08 | B |
| ATOM | 6876 | O | ALA | B | 486 | −66.841 | 43.917 | 41.082 | 1.00 | 75.99 | B |
| ATOM | 6877 | N | ASP | B | 487 | −65.309 | 42.265 | 40.938 | 1.00 | 76.58 | B |
| ATOM | 6878 | CA | ASP | B | 487 | −65.876 | 41.784 | 39.680 | 1.00 | 75.99 | B |
| ATOM | 6879 | CB | ASP | B | 487 | −64.859 | 40.979 | 38.875 | 1.00 | 74.65 | B |
| ATOM | 6880 | CG | ASP | B | 487 | −64.337 | 39.817 | 39.623 | 1.00 | 73.69 | B |
| ATOM | 6881 | OD1 | ASP | B | 487 | −65.056 | 39.364 | 40.534 | 1.00 | 73.77 | B |
| ATOM | 6882 | OD2 | ASP | B | 487 | −63.221 | 39.359 | 39.291 | 1.00 | 73.64 | B |
| ATOM | 6883 | C | ASP | B | 487 | −67.156 | 40.983 | 39.874 | 1.00 | 75.88 | B |
| ATOM | 6884 | O | ASP | B | 487 | −67.774 | 40.543 | 38.903 | 1.00 | 75.32 | B |
| ATOM | 6885 | N | LEU | B | 488 | −67.548 | 40.787 | 41.130 | 1.00 | 75.54 | B |
| ATOM | 6886 | CA | LEU | B | 488 | −68.802 | 40.107 | 41.405 | 1.00 | 75.45 | B |
| ATOM | 6887 | CB | LEU | B | 488 | −68.856 | 39.589 | 42.845 | 1.00 | 74.84 | B |
| ATOM | 6888 | CG | LEU | B | 488 | −69.307 | 38.130 | 42.996 | 1.00 | 75.58 | B |
| ATOM | 6889 | CD1 | LEU | B | 488 | −68.198 | 37.199 | 42.470 | 1.00 | 74.03 | B |
| ATOM | 6890 | CD2 | LEU | B | 488 | −69.615 | 37.821 | 44.462 | 1.00 | 74.48 | B |
| ATOM | 6891 | C | LEU | B | 488 | −69.779 | 41.265 | 41.229 | 1.00 | 75.84 | B |
| ATOM | 6892 | O | LEU | B | 488 | −70.828 | 41.129 | 40.606 | 1.00 | 75.96 | B |
| ATOM | 6893 | N | VAL | B | 489 | −69.400 | 42.417 | 41.774 | 1.00 | 76.14 | B |
| ATOM | 6894 | CA | VAL | B | 489 | −70.215 | 43.613 | 41.657 | 1.00 | 77.63 | B |
| ATOM | 6895 | CB | VAL | B | 489 | −69.547 | 44.838 | 42.350 | 1.00 | 77.94 | B |
| ATOM | 6896 | CG1 | VAL | B | 489 | −70.141 | 46.152 | 41.825 | 1.00 | 75.60 | B |
| ATOM | 6897 | CG2 | VAL | B | 489 | −69.759 | 44.754 | 43.856 | 1.00 | 79.08 | B |
| ATOM | 6898 | C | VAL | B | 489 | −70.352 | 43.907 | 40.176 | 1.00 | 78.65 | B |
| ATOM | 6899 | O | VAL | B | 489 | −71.446 | 44.185 | 39.691 | 1.00 | 79.99 | B |
| ATOM | 6900 | N | ILE | B | 490 | −69.234 | 43.832 | 39.461 | 1.00 | 79.37 | B |
| ATOM | 6901 | CA | ILE | B | 490 | −69.222 | 44.103 | 38.030 | 1.00 | 79.92 | B |
| ATOM | 6902 | CB | ILE | B | 490 | −67.808 | 43.929 | 37.440 | 1.00 | 77.81 | B |
| ATOM | 6903 | CG2 | ILE | B | 490 | −67.878 | 43.812 | 35.924 | 1.00 | 76.93 | B |
| ATOM | 6904 | CG1 | ILE | B | 490 | −66.938 | 45.121 | 37.870 | 1.00 | 76.77 | B |
| ATOM | 6905 | CD1 | ILE | B | 490 | −65.509 | 45.105 | 37.343 | 1.00 | 74.12 | B |
| ATOM | 6906 | C | ILE | B | 490 | −70.209 | 43.222 | 37.288 | 1.00 | 82.26 | B |
| ATOM | 6907 | O | ILE | B | 490 | −70.804 | 43.654 | 36.302 | 1.00 | 82.52 | B |
| ATOM | 6908 | N | SER | B | 491 | −70.396 | 41.996 | 37.769 | 1.00 | 85.22 | B |
| ATOM | 6909 | CA | SER | B | 491 | −71.349 | 41.082 | 37.141 | 1.00 | 88.03 | B |
| ATOM | 6910 | CB | SER | B | 491 | −71.182 | 39.659 | 37.676 | 1.00 | 88.66 | B |
| ATOM | 6911 | OG | SER | B | 491 | −72.071 | 38.775 | 37.015 | 1.00 | 89.47 | B |
| ATOM | 6912 | C | SER | B | 491 | −72.760 | 41.571 | 37.437 | 1.00 | 89.06 | B |
| ATOM | 6913 | O | SER | B | 491 | −73.617 | 41.577 | 36.560 | 1.00 | 89.62 | B |
| ATOM | 6914 | N | TYR | B | 492 | −72.996 | 41.963 | 38.685 | 1.00 | 90.30 | B |
| ATOM | 6915 | CA | TYR | B | 492 | −74.295 | 42.484 | 39.084 | 1.00 | 92.98 | B |
| ATOM | 6916 | CB | TYR | B | 492 | −74.312 | 42.735 | 40.597 | 1.00 | 92.50 | B |
| ATOM | 6917 | CG | TYR | B | 492 | −75.393 | 43.681 | 41.078 | 1.00 | 92.99 | B |
| ATOM | 6918 | CD1 | TYR | B | 492 | −76.569 | 43.205 | 41.669 | 1.00 | 92.28 | B |
| ATOM | 6919 | CE1 | TYR | B | 492 | −77.539 | 44.090 | 42.157 | 1.00 | 92.63 | B |
| ATOM | 6920 | CD2 | TYR | B | 492 | −75.219 | 45.063 | 40.977 | 1.00 | 94.12 | B |
| ATOM | 6921 | CE2 | TYR | B | 492 | −76.177 | 45.953 | 41.453 | 1.00 | 94.87 | B |
| ATOM | 6922 | CZ | TYR | B | 492 | −77.330 | 45.467 | 42.045 | 1.00 | 94.65 | B |
| ATOM | 6923 | OH | TYR | B | 492 | −78.242 | 46.380 | 42.536 | 1.00 | 95.46 | B |
| ATOM | 6924 | C | TYR | B | 492 | −74.506 | 43.792 | 38.311 | 1.00 | 94.92 | B |
| ATOM | 6925 | O | TYR | B | 492 | −75.624 | 44.110 | 37.886 | 1.00 | 95.49 | B |
| ATOM | 6926 | N | LEU | B | 493 | −73.422 | 44.547 | 38.138 | 1.00 | 96.66 | B |
| ATOM | 6927 | CA | LEU | B | 493 | −73.459 | 45.808 | 37.398 | 1.00 | 98.08 | B |
| ATOM | 6928 | CB | LEU | B | 493 | −72.037 | 46.352 | 37.224 | .00 | 97.91 | B |
| ATOM | 6929 | CG | LEU | B | 493 | −71.602 | 46.887 | 35.853 | .00 | 97.96 | B |
| ATOM | 6930 | CD1 | LEU | B | 493 | −72.426 | 48.104 | 35.467 | .00 | 97.95 | B |
| ATOM | 6931 | CD2 | LEU | B | 493 | −70.126 | 47.243 | 35.905 | .00 | 97.95 | B |
| ATOM | 6932 | C | LEU | B | 493 | −74.067 | 45.477 | 36.043 | 1.00 | 98.87 | B |
| ATOM | 6933 | O | LEU | B | 493 | −74.828 | 46.258 | 35.468 | 1.00 | 99.22 | B |
| ATOM | 6934 | N | GLU | B | 494 | −73.712 | 44.296 | 35.551 | 1.00 | 99.47 | B |
| ATOM | 6935 | CA | GLU | B | 494 | −74.207 | 43.785 | 34.285 | 1.00 | 99.84 | B |
| ATOM | 6936 | CB | GLU | B | 494 | −73.074 | 43.089 | 33.525 | 1.00 | 99.64 | B |
| ATOM | 6937 | CG | GLU | B | 494 | −72.044 | 44.043 | 32.937 | 1.00 | 99.47 | B |
| ATOM | 6938 | CD | GLU | B | 494 | −70.753 | 43.340 | 32.558 | 1.00 | 99.91 | B |
| ATOM | 6939 | OE1 | GLU | B | 494 | −70.823 | 42.281 | 31.898 | 1.00 | 99.35 | B |
| ATOM | 6940 | OE2 | GLU | B | 494 | −69.668 | 43.850 | 32.919 | 1.00 | 100.00 | B |
| ATOM | 6941 | C | GLU | B | 494 | −75.332 | 42.797 | 34.596 | 1.00 | 100.00 | B |
| ATOM | 6942 | O | GLU | B | 494 | −76.173 | 43.060 | 35.460 | 1.00 | 100.00 | B |
| ATOM | 6943 | N | SER | B | 495 | −75.341 | 41.660 | 33.904 | 1.00 | 100.00 | B |
| ATOM | 6944 | CA | SER | B | 495 | −76.372 | 40.646 | 34.114 | 1.00 | 100.00 | B |
| ATOM | 6945 | CB | SER | B | 495 | −76.186 | 39.972 | 35.482 | 1.00 | 100.00 | B |
| ATOM | 6946 | OG | SER | B | 495 | −77.137 | 38.937 | 35.681 | 1.00 | 100.00 | B |
| ATOM | 6947 | C | SER | B | 495 | −77.760 | 41.280 | 34.031 | 1.00 | 100.00 | B |
| ATOM | 6948 | O | SER | B | 495 | −78.754 | 40.677 | 34.446 | 1.00 | 100.00 | B |
| ATOM | 6949 | N | VAL | B | 496 | −77.811 | 42.502 | 33.494 | 1.00 | 100.00 | B |
| ATOM | 6950 | CA | VAL | B | 496 | −79.059 | 43.250 | 33.331 | 1.00 | 100.00 | B |
| ATOM | 6951 | CB | VAL | B | 496 | −78.902 | 44.719 | 33.853 | 1.00 | 99.66 | B |
| ATOM | 6952 | CG1 | VAL | B | 496 | −80.193 | 45.502 | 33.633 | 1.00 | 99.24 | B |
| ATOM | 6953 | CG2 | VAL | B | 496 | −78.551 | 44.709 | 35.343 | 1.00 | 98.03 | B |
| ATOM | 6954 | C | VAL | B | 496 | −79.477 | 43.242 | 31.847 | 1.00 | 99.88 | B |
| ATOM | 6955 | O | VAL | B | 496 | −79.540 | 44.284 | 31.188 | 1.00 | 99.37 | B |
| ATOM | 6956 | N | SER | B | 497 | −79.766 | 42.043 | 31.345 | 1.00 | 99.66 | B |
| ATOM | 6957 | CA | SER | B | 497 | −80.168 | 41.838 | 29.960 | 1.00 | 99.90 | B |
| ATOM | 6958 | CB | SER | B | 497 | −81.502 | 42.539 | 29.683 | 1.00 | 100.00 | B |
| ATOM | 6959 | OG | SER | B | 497 | −81.890 | 42.392 | 28.325 | 1.00 | 100.00 | B |
| ATOM | 6960 | C | SER | B | 497 | −79.101 | 42.367 | 29.008 | 1.00 | 100.00 | B |
| ATOM | 6961 | O | SER | B | 497 | −79.476 | 42.814 | 27.898 | 1.00 | 100.00 | B |
| ATOM | 6962 | OXT | SER | B | 497 | −77.905 | 42.304 | 29.381 | 1.00 | 99.54 | B |
| ATOM | 6963 | O | HOH | S | 1 | −11.384 | 13.913 | 3.362 | 1.00 | 74.94 | S |
| ATOM | 6964 | O | HOH | S | 5 | −6.594 | 16.179 | 3.588 | 1.00 | 56.71 | S |
| ATOM | 6965 | O | HOH | S | 6 | −4.811 | 19.521 | 7.115 | 1.00 | 36.03 | S |
| ATOM | 6966 | O | HOH | S | 7 | −30.924 | 13.584 | 8.604 | 1.00 | 55.23 | S |
| ATOM | 6967 | O | HOH | S | 8 | −27.998 | −.460 | 19.146 | 1.00 | 24.55 | S |
| ATOM | 6968 | O | HOH | S | 9 | −12.328 | −5.487 | 29.353 | 1.00 | 55.61 | S |
| ATOM | 6969 | O | HOH | S | 11 | −19.496 | 3.989 | 27.504 | 1.00 | 53.47 | S |
| ATOM | 6970 | O | HOH | S | 15 | −6.201 | 4.415 | 5.445 | 1.00 | 51.09 | S |
| ATOM | 6971 | O | HOH | S | 18 | −15.729 | 19.122 | 22.218 | 1.00 | 56.61 | S |
| ATOM | 6972 | O | HOH | S | 19 | −6.250 | 8.318 | 7.872 | 1.00 | 54.78 | S |
| ATOM | 6973 | O | HOH | S | 20 | −22.252 | −9.353 | 15.514 | 1.00 | 49.74 | S |
| ATOM | 6974 | O | HOH | S | 22 | −30.570 | 13.627 | 5.382 | 1.00 | 68.26 | S |
| ATOM | 6975 | O | HOH | S | 24 | −51.487 | −22.002 | 38.497 | 1.00 | 65.23 | S |
| ATOM | 6976 | O | HOH | S | 25 | −52.166 | −21.116 | 40.854 | 1.00 | 63.66 | S |
| ATOM | 6977 | O | HOH | S | 26 | −53.033 | .412 | 40.585 | 1.00 | 53.14 | S |
| ATOM | 6978 | O | HOH | S | 27 | −44.263 | .522 | 41.550 | 1.00 | 47.86 | S |
| ATOM | 6979 | O | HOH | S | 30 | −39.614 | 18.643 | 21.833 | 1.00 | 46.36 | S |
| ATOM | 6980 | O | HOH | S | 31 | −33.506 | .080 | 4.601 | 1.00 | 65.44 | S |
| ATOM | 6981 | O | HOH | S | 32 | −28.356 | 13.335 | 4.302 | 1.00 | 68.08 | S |
| ATOM | 6982 | O | HOH | S | 36 | −64.085 | −13.038 | 26.260 | 1.00 | 44.50 | S |
| ATOM | 6983 | O | HOH | S | 38 | −56.937 | −5.236 | 8.138 | 1.00 | 55.69 | S |
| ATOM | 6984 | O | HOH | S | 39 | −57.826 | −10.166 | 8.046 | 1.00 | 52.17 | S |
| ATOM | 6985 | O | HOH | S | 40 | −27.197 | −13.975 | 18.648 | 1.00 | 57.77 | S |
| ATOM | 6986 | O | HOH | S | 41 | −15.781 | 3.759 | 33.575 | 1.00 | 58.24 | S |
| ATOM | 6987 | O | HOH | S | 42 | −28.227 | 1.976 | 34.987 | 1.00 | 52.11 | S |
| ATOM | 6988 | O | HOH | S | 43 | −32.250 | −4.103 | 8.737 | 1.00 | 63.09 | S |
| ATOM | 6989 | O | HOH | S | 44 | −16.224 | 10.197 | 6.554 | 1.00 | 55.62 | S |
| ATOM | 6990 | O | HOH | S | 46 | −20.700 | −12.761 | 36.445 | 1.00 | 53.48 | S |
| ATOM | 6991 | O | HOH | S | 49 | −57.329 | −26.383 | 28.802 | 1.00 | 46.20 | S |
| ATOM | 6992 | O | HOH | S | 50 | −57.757 | −27.752 | 26.669 | 1.00 | 32.71 | S |
| ATOM | 6993 | O | HOH | S | 51 | −21.644 | −15.800 | 19.973 | 1.00 | 58.75 | S |
| ATOM | 6994 | O | HOH | S | 52 | −21.724 | −17.519 | 23.060 | 1.00 | 52.80 | S |
| ATOM | 6995 | O | HOH | S | 53 | −20.048 | −8.772 | 36.489 | 1.00 | 50.05 | S |
| ATOM | 6996 | O | HOH | S | 55 | −32.248 | .319 | 31.608 | 1.00 | 62.20 | S |
| ATOM | 6997 | O | HOH | S | 58 | −30.699 | −3.889 | 35.608 | 1.00 | 44.07 | S |
| ATOM | 6998 | O | HOH | S | 60 | −27.125 | 15.250 | 30.857 | 1.00 | 52.79 | S |
| ATOM | 6999 | O | HOH | S | 61 | −24.709 | 15.476 | 31.743 | 1.00 | 54.91 | S |
| ATOM | 7000 | O | HOH | S | 63 | 1.006 | .872 | 29.653 | 1.00 | 63.21 | S |
| ATOM | 7001 | O | HOH | S | 66 | −3.578 | −10.432 | 35.145 | 1.00 | 90.84 | S |
| ATOM | 7002 | O | HOH | S | 68 | −27.284 | −15.873 | 34.517 | 1.00 | 57.49 | S |
| ATOM | 7003 | O | HOH | S | 71 | −16.887 | −14.619 | 34.473 | 1.00 | 56.68 | S |
| ATOM | 7004 | O | HOH | S | 72 | −29.736 | −21.015 | 33.771 | 1.00 | 47.78 | S |
| ATOM | 7005 | O | HOH | S | 74 | −40.260 | −20.514 | 41.776 | 1.00 | 36.33 | S |
| ATOM | 7006 | O | HOH | S | 75 | −45.199 | −16.277 | 47.060 | 1.00 | 61.75 | S |
| ATOM | 7007 | O | HOH | S | 76 | −44.704 | −15.208 | 49.886 | 1.00 | 53.84 | S |
| ATOM | 7008 | O | HOH | S | 77 | −41.864 | −1.210 | 18.134 | 1.00 | 36.54 | S |
| ATOM | 7009 | O | HOH | S | 78 | −43.286 | −25.883 | 20.729 | 1.00 | 50.17 | S |
| ATOM | 7010 | O | HOH | S | 79 | −11.803 | 7.229 | 31.753 | 1.00 | 63.57 | S |
| ATOM | 7011 | O | HOH | S | 80 | −5.736 | .314 | 25.529 | 1.00 | 82.34 | S |
| ATOM | 7012 | O | HOH | S | 81 | −20.761 | 15.902 | 20.696 | 1.00 | 59.05 | S |
| ATOM | 7013 | O | HOH | S | 82 | −29.672 | 11.035 | 32.136 | 1.00 | 45.90 | S |
| ATOM | 7014 | O | HOH | S | 83 | −50.558 | −16.832 | 33.998 | 1.00 | 40.20 | S |
| ATOM | 7015 | O | HOH | S | 84 | −53.472 | −20.214 | 36.945 | 1.00 | 73.31 | S |
| ATOM | 7016 | O | HOH | S | 85 | −58.240 | −.964 | 47.627 | 1.00 | 55.26 | S |
| ATOM | 7017 | O | HOH | S | 86 | −30.881 | 6.453 | 59.168 | 1.00 | 43.54 | S |
| ATOM | 7018 | O | HOH | S | 87 | −36.225 | 6.056 | 53.652 | 1.00 | 57.17 | S |
| ATOM | 7019 | O | HOH | S | 88 | −39.963 | −.148 | 18.344 | 1.00 | 92.32 | S |
| ATOM | 7020 | O | HOH | S | 89 | −45.532 | 1.693 | 9.069 | 1.00 | 53.49 | S |
| ATOM | 7021 | O | HOH | S | 90 | −25.748 | 14.609 | 2.642 | 1.00 | 50.86 | S |
| ATOM | 7022 | O | HOH | S | 101 | −45.041 | 37.337 | 60.749 | 1.00 | 61.70 | S |
| ATOM | 7023 | O | HOH | S | 102 | −71.489 | 54.649 | 32.668 | 1.00 | 58.30 | S |
| ATOM | 7024 | O | HOH | S | 103 | −70.820 | 61.011 | 35.904 | 1.00 | 69.76 | S |
| ATOM | 7025 | O | HOH | S | 104 | −45.466 | 53.428 | 45.959 | 1.00 | 61.74 | S |
| ATOM | 7026 | O | HOH | S | 105 | −73.098 | 60.870 | 34.900 | 1.00 | 87.02 | S |
| ATOM | 7027 | O | HOH | S | 106 | −75.161 | 56.127 | 35.070 | 1.00 | 54.67 | S |
| ATOM | 7028 | O | HOH | S | 107 | −44.084 | 19.296 | 77.708 | 1.00 | 47.62 | S |
| ATOM | 7029 | O | HOH | S | 108 | −56.019 | 45.427 | 55.852 | 1.00 | 78.60 | S |
| ATOM | 7030 | O | HOH | S | 109 | −79.744 | 38.687 | 62.912 | 1.00 | 70.07 | S |
| ATOM | 7031 | O | HOH | S | 110 | −41.507 | 56.358 | 45.160 | 1.00 | 51.99 | S |
| ATOM | 7032 | O | HOH | S | 112 | −44.125 | 21.171 | 59.641 | 1.00 | 54.60 | S |
| ATOM | 7033 | O | HOH | S | 113 | −40.467 | 18.380 | 53.118 | 1.00 | 60.01 | S |
| ATOM | 7034 | O | HOH | S | 114 | −55.744 | 10.817 | 71.357 | 1.00 | 46.25 | S |
| ATOM | 7035 | O | HOH | S | 115 | −74.387 | 59.087 | 46.397 | 1.00 | 65.78 | S |
| ATOM | 7036 | O | HOH | S | 116 | −42.583 | 30.762 | 38.378 | 1.00 | 67.53 | S |
| ATOM | 7037 | O | HOH | S | 118 | −68.742 | 40.592 | 36.060 | 1.00 | 74.33 | S |
| ATOM | 7038 | O | HOH | S | 119 | −74.293 | 53.898 | 34.458 | 1.00 | 54.39 | S |
| ATOM | 7039 | O | HOH | S | 120 | −62.855 | 49.835 | 62.831 | 1.00 | 55.69 | S |
| ATOM | 7040 | O | HOH | S | 123 | −45.860 | 48.136 | 49.606 | 1.00 | 100.00 | S |
| ATOM | 7041 | O | HOH | S | 125 | −34.617 | 22.918 | 85.317 | 1.00 | 63.19 | S |
| ATOM | 7042 | O | HOH | S | 126 | −35.094 | 22.207 | 58.216 | 1.00 | 71.08 | S |
| ATOM | 7043 | O | HOH | S | 127 | −42.007 | 22.841 | 59.208 | 1.00 | 73.57 | S |
| ATOM | 7044 | O | HOH | S | 130 | −42.957 | 36.730 | 39.893 | 1.00 | 65.65 | S |
| ATOM | 7045 | O | HOH | S | 131 | −45.779 | 54.145 | 52.839 | 1.00 | 65.63 | S |
| ATOM | 7046 | O | HOH | S | 132 | −50.448 | 58.848 | 41.295 | 1.00 | 66.07 | S |
| ATOM | 7047 | O | HOH | S | 133 | −59.385 | 57.803 | 42.031 | 1.00 | 63.76 | S |
| ATOM | 7048 | O | HOH | S | 135 | −76.408 | 35.037 | 69.261 | 1.00 | 62.05 | S |
| ATOM | 7049 | O | HOH | S | 136 | −23.430 | 38.928 | 72.925 | 1.00 | 79.99 | S |
| ATOM | 7050 | O | HOH | S | 137 | −22.407 | 37.909 | 75.773 | 1.00 | 52.23 | S |
| ATOM | 7051 | O | HOH | S | 138 | −26.854 | 52.710 | 62.762 | 1.00 | 82.86 | S |
| ATOM | 7052 | O | HOH | S | 139 | −26.215 | 53.924 | 69.307 | 1.00 | 47.98 | S |
| ATOM | 7053 | O | HOH | S | 140 | −59.392 | 48.490 | 68.933 | 1.00 | 59.28 | S |
| ATOM | 7054 | O | HOH | S | 144 | −64.196 | 59.584 | 40.911 | 1.00 | 48.70 | S |
| ATOM | 7055 | O | HOH | S | 147 | −51.247 | 43.061 | 68.408 | 1.00 | 100.00 | S |
| ATOM | 7056 | O | HOH | S | 149 | −31.011 | 35.119 | 84.971 | 1.00 | 46.17 | S |
| ATOM | 7057 | O | HOH | S | 150 | −28.011 | 36.915 | 84.631 | 1.00 | 71.97 | S |
| ATOM | 7058 | O | HOH | S | 151 | −62.722 | 48.486 | 67.828 | 1.00 | 43.11 | S |
| ATOM | 7059 | O | HOH | S | 152 | −62.601 | 46.520 | 73.322 | 1.00 | 54.03 | S |
| ATOM | 7060 | O | HOH | S | 156 | −63.224 | 28.528 | 52.162 | 1.00 | 73.79 | S |
| ATOM | 7061 | O | HOH | S | 158 | −57.290 | 29.361 | 62.476 | 1.00 | 70.85 | S |
| ATOM | 7062 | O | HOH | S | 159 | −55.360 | 50.029 | 54.609 | 1.00 | 35.73 | S |
| ATOM | 7063 | O | HOH | S | 160 | −42.789 | 44.899 | 63.663 | 1.00 | 40.12 | S |
| ATOM | 7064 | O | HOH | S | 161 | −44.118 | 39.384 | 38.892 | 1.00 | 60.90 | S |
| ATOM | 7065 | O | HOH | S | 162 | −42.416 | 52.683 | 49.444 | 1.00 | 62.15 | S |
| ATOM | 7066 | O | HOH | S | 163 | −38.881 | 15.343 | 64.226 | 1.00 | 48.03 | S |
| ATOM | 7067 | O | HOH | S | 164 | −31.650 | 17.595 | 64.271 | 1.00 | 36.27 | S |
| ATOM | 7068 | O | HOH | S | 165 | −35.747 | 7.148 | 71.042 | 1.00 | 62.30 | S |
| ATOM | 7069 | O | HOH | S | 166 | −36.313 | 8.237 | 65.996 | 1.00 | 42.40 | S |
| ATOM | 7070 | O | HOH | S | 167 | −37.124 | 16.883 | 64.087 | 1.00 | 79.73 | S |
| ATOM | 7071 | O | HOH | S | 168 | −31.659 | 9.284 | 67.115 | 1.00 | 63.42 | S |
| ATOM | 7072 | O | HOH | S | 169 | −42.906 | 27.949 | 45.841 | 1.00 | 56.86 | S |
| ATOM | 7073 | O | HOH | S | 170 | −42.592 | 44.490 | 57.926 | 1.00 | 51.73 | S |
| ATOM | 7074 | O | HOH | S | 171 | −87.375 | 52.109 | 39.952 | 1.00 | 89.55 | S |
| ATOM | 7075 | O | HOH | S | 172 | −72.807 | 66.418 | 54.046 | 1.00 | 73.88 | S |
| ATOM | 7076 | O | HOH | S | 173 | −23.849 | 50.585 | 76.016 | 1.00 | 57.87 | S |
| ATOM | 7077 | O | HOH | S | 174 | −21.130 | 52.198 | 75.322 | 1.00 | 63.57 | S |
| ATOM | 7078 | O | HOH | S | 175 | −23.397 | 47.186 | 79.518 | 1.00 | 67.76 | S |
| ATOM | 7079 | O | HOH | S | 176 | −52.323 | 24.109 | 84.731 | 1.00 | 38.85 | S |
| ATOM | 7080 | O | HOH | S | 208 | −40.894 | 8.300 | 48.188 | 1.00 | 77.53 | S |
| ATOM | 7081 | O | HOH | S | 210 | −41.492 | .614 | 45.927 | 1.00 | 46.30 | S |
| ATOM | 7082 | O | HOH | S | 214 | −39.147 | 4.509 | 45.967 | 1.00 | 38.40 | S |
| ATOM | 7083 | O | HOH | S | 215 | −39.673 | 2.044 | 47.084 | 1.00 | 49.08 | S |
| ATOM | 7084 | O | HOH | S | 216 | −39.357 | 2.024 | 44.742 | 1.00 | 49.84 | S |
| ATOM | 7085 | O | HOH | S | 217 | −39.412 | −.089 | 45.737 | 1.00 | 27.55 | S |
| ATOM | 7086 | O | HOH | S | 218 | −41.233 | 8.437 | 45.729 | 1.00 | 41.66 | S |
| ATOM | 7087 | O | HOH | S | 219 | −43.094 | 8.453 | 47.429 | 1.00 | 40.36 | S |
| ATOM | 7088 | O | HOH | S | 220 | −42.515 | 6.352 | 48.678 | 1.00 | 54.08 | S |
| ATOM | 7089 | O | HOH | S | 221 | −44.490 | 7.421 | 49.569 | 1.00 | 43.28 | S |
| ATOM | 7090 | O | HOH | S | 223 | −41.747 | 3.219 | 46.895 | 1.00 | 51.01 | S |
| ATOM | 7091 | O | HOH | S | 224 | −44.095 | 5.447 | 50.963 | 1.00 | 50.58 | S |
| ATOM | 7092 | O | HOH | S | 225 | −46.381 | 6.400 | 50.868 | 1.00 | 45.59 | S |
| ATOM | 7093 | O | HOH | S | 226 | −44.557 | 7.655 | 52.508 | 1.00 | 37.19 | S |
| ATOM | 7094 | O | HOH | S | 228 | −44.970 | 5.210 | 48.690 | 1.00 | 96.85 | S |
| ATOM | 7095 | O | HOH | S | 258 | −46.357 | 13.881 | 53.974 | 1.00 | 53.46 | S |
| ATOM | 7096 | O | HOH | S | 260 | −47.079 | 17.425 | 58.110 | 1.00 | 66.88 | S |
| ATOM | 7097 | O | HOH | S | 264 | −47.846 | 17.074 | 55.051 | 1.00 | 83.18 | S |
| ATOM | 7098 | O | HOH | S | 266 | −47.986 | 17.267 | 60.528 | 1.00 | 49.74 | S |
| ATOM | 7099 | O | HOH | S | 268 | −45.577 | 16.216 | 52.906 | 1.00 | 43.41 | S |
| ATOM | 7100 | O | HOH | S | 269 | −45.071 | 15.695 | 55.154 | 1.00 | 44.87 | S |
| ATOM | 7101 | O | HOH | S | 270 | −43.667 | 13.646 | 53.612 | 1.00 | 43.74 | S |
| ATOM | 7102 | O | HOH | S | 271 | −42.177 | 11.986 | 55.529 | 1.00 | 36.47 | S |
| ATOM | 7103 | O | HOH | S | 273 | −49.306 | 16.874 | 57.396 | 1.00 | 49.42 | S |
| ATOM | 7104 | O | HOH | S | 275 | −44.707 | 11.699 | 57.245 | 1.00 | 39.66 | S |
| ATOM | 7105 | O | HOH | S | 278 | −44.273 | 13.402 | 55.913 | 1.00 | 36.83 | S |
| ATOM | 7106 | AP | FAD | A | 600 | −25.052 | −2.876 | 19.915 | 1.00 | 42.03 | A |
| ATOM | 7107 | AO1 | FAD | A | 600 | −24.849 | −4.171 | 20.580 | 1.00 | 44.95 | A |
| ATOM | 7108 | AO2 | FAD | A | 600 | −26.388 | −2.505 | 19.427 | 1.00 | 43.63 | A |
| ATOM | 7109 | AO5* | FAD | A | 600 | −24.064 | −2.727 | 18.674 | 1.00 | 43.66 | A |
| ATOM | 7110 | AC5* | FAD | A | 600 | −22.656 | −3.030 | 18.758 | 1.00 | 45.36 | A |
| ATOM | 7111 | AC4* | FAD | A | 600 | −22.217 | −3.355 | 17.318 | 1.00 | 46.97 | A |
| ATOM | 7112 | AO4* | FAD | A | 600 | −20.894 | −3.842 | 17.342 | 1.00 | 48.76 | A |
| ATOM | 7113 | AC3* | FAD | A | 600 | −23.031 | −4.441 | 16.622 | 1.00 | 48.16 | A |
| ATOM | 7114 | AO3* | FAD | A | 600 | −23.758 | −3.931 | 15.536 | 1.00 | 49.79 | A |
| ATOM | 7115 | AC2* | FAD | A | 600 | −22.000 | −5.468 | 16.248 | 1.00 | 49.28 | A |
| ATOM | 7116 | AO2* | FAD | A | 600 | −22.268 | −6.223 | 15.091 | 1.00 | 50.49 | A |
| ATOM | 7117 | AC1* | FAD | A | 600 | −20.728 | −4.627 | 16.190 | 1.00 | 49.54 | A |
| ATOM | 7118 | AN9 | FAD | A | 600 | −19.450 | −5.365 | 16.321 | 1.00 | 50.36 | A |
| ATOM | 7119 | AC8 | FAD | A | 600 | −19.135 | −6.335 | 17.242 | 1.00 | 51.61 | A |
| ATOM | 7120 | AN7 | FAD | A | 600 | −17.901 | −6.800 | 17.099 | 1.00 | 50.42 | A |
| ATOM | 7121 | AC5 | FAD | A | 600 | −17.391 | −6.103 | 16.038 | 1.00 | 50.53 | A |
| ATOM | 7122 | AC6 | FAD | A | 600 | −16.130 | −6.138 | 15.402 | 1.00 | 49.69 | A |
| ATOM | 7123 | AN6 | FAD | A | 600 | −15.138 | −6.923 | 15.761 | 1.00 | 48.85 | A |
| ATOM | 7124 | AN1 | FAD | A | 600 | −15.941 | −5.294 | 14.340 | 1.00 | 50.91 | A |
| ATOM | 7125 | AC2 | FAD | A | 600 | −16.956 | −4.458 | 13.935 | 1.00 | 51.28 | A |
| ATOM | 7126 | AN3 | FAD | A | 600 | −18.178 | −4.372 | 14.500 | 1.00 | 49.43 | A |
| ATOM | 7127 | AC4 | FAD | A | 600 | −18.349 | −5.208 | 15.544 | 1.00 | 50.29 | A |
| ATOM | 7128 | N1 | FAD | A | 600 | −33.435 | −.762 | 25.304 | 1.00 | 40.64 | A |
| ATOM | 7129 | C2 | FAD | A | 600 | −34.452 | .141 | 25.563 | 1.00 | 42.03 | A |
| ATOM | 7130 | O2 | FAD | A | 600 | −34.206 | 1.295 | 25.911 | 1.00 | 44.05 | A |
| ATOM | 7131 | N3 | FAD | A | 600 | −35.783 | −.295 | 25.406 | 1.00 | 39.06 | A |
| ATOM | 7132 | C4 | FAD | A | 600 | −36.139 | −1.568 | 25.012 | 1.00 | 40.17 | A |
| ATOM | 7133 | O4 | FAD | A | 600 | −37.340 | −1.875 | 24.885 | 1.00 | 44.45 | A |
| ATOM | 7134 | C4A | FAD | A | 600 | −35.093 | −2.493 | 24.747 | 1.00 | 39.39 | A |
| ATOM | 7135 | N5 | FAD | A | 600 | −35.372 | −3.808 | 24.325 | 1.00 | 37.90 | A |
| ATOM | 7136 | C5A | FAD | A | 600 | −34.333 | −4.696 | 24.024 | 1.00 | 36.51 | A |
| ATOM | 7137 | C6 | FAD | A | 600 | −34.617 | −5.989 | 23.577 | 1.00 | 32.62 | A |
| ATOM | 7138 | C7 | FAD | A | 600 | −33.602 | −6.900 | 23.232 | 1.00 | 30.69 | A |
| ATOM | 7139 | C7M | FAD | A | 600 | −33.963 | −8.274 | 22.748 | 1.00 | 27.68 | A |
| ATOM | 7140 | C8 | FAD | A | 600 | −32.236 | −6.479 | 23.350 | 1.00 | 32.76 | A |
| ATOM | 7141 | C8M | FAD | A | 600 | −31.097 | −7.414 | 22.988 | 1.00 | 35.56 | A |
| ATOM | 7142 | C9 | FAD | A | 600 | −31.922 | −5.176 | 23.813 | 1.00 | 32.84 | A |
| ATOM | 7143 | C9A | FAD | A | 600 | −32.952 | −4.261 | 24.157 | 1.00 | 36.52 | A |
| ATOM | 7144 | N10 | FAD | A | 600 | −32.687 | −2.921 | 24.618 | 1.00 | 39.03 | A |
| ATOM | 7145 | C10 | FAD | A | 600 | −33.745 | −2.043 | 24.902 | 1.00 | 39.19 | A |
| ATOM | 7146 | C1* | FAD | A | 600 | −31.289 | −2.404 | 24.799 | 1.00 | 40.11 | A |
| ATOM | 7147 | C2* | FAD | A | 600 | −30.753 | −1.613 | 23.632 | 1.00 | 41.68 | A |
| ATOM | 7148 | O2* | FAD | A | 600 | −31.160 | −2.242 | 22.444 | 1.00 | 41.76 | A |
| ATOM | 7149 | C3* | FAD | A | 600 | −29.218 | −1.565 | 23.753 | 1.00 | 42.48 | A |
| ATOM | 7150 | O3* | FAD | A | 600 | −28.806 | −.930 | 24.966 | 1.00 | 43.50 | A |
| ATOM | 7151 | C4* | FAD | A | 600 | −28.487 | −.790 | 22.642 | 1.00 | 42.60 | A |
| ATOM | 7152 | O4* | FAD | A | 600 | −28.941 | −1.151 | 21.347 | 1.00 | 43.39 | A |
| ATOM | 7153 | C5* | FAD | A | 600 | −26.991 | −1.034 | 22.733 | 1.00 | 42.33 | A |
| ATOM | 7154 | O5* | FAD | A | 600 | −26.439 | −.318 | 21.647 | 1.00 | 41.84 | A |
| ATOM | 7155 | P | FAD | A | 600 | −24.926 | −.348 | 21.280 | 1.00 | 41.75 | A |
| ATOM | 7156 | O1P | FAD | A | 600 | −24.823 | .508 | 20.084 | 1.00 | 40.93 | A |
| ATOM | 7157 | O2P | FAD | A | 600 | −24.080 | .063 | 22.456 | 1.00 | 43.59 | A |
| ATOM | 7158 | O3P | FAD | A | 600 | −24.555 | −1.826 | 20.984 | 1.00 | 40.83 | A |
| ATOM | 7159 | AP | FAD | B | 600 | −59.263 | 45.292 | 58.016 | 1.00 | 65.18 | B |
| ATOM | 7160 | AO1 | FAD | B | 600 | −59.684 | 45.207 | 59.435 | 1.00 | 63.09 | B |
| ATOM | 7161 | AO2 | FAD | B | 600 | −57.838 | 45.476 | 57.673 | 1.00 | 65.77 | B |
| ATOM | 7162 | AO5* | FAD | B | 600 | −60.047 | 46.448 | 57.278 | 1.00 | 64.73 | B |
| ATOM | 7163 | AC5* | FAD | B | 600 | −61.468 | 46.577 | 57.405 | 1.00 | 64.70 | B |
| ATOM | 7164 | AC4* | FAD | B | 600 | −61.712 | 48.080 | 57.390 | 1.00 | 65.67 | B |
| ATOM | 7165 | AO4* | *FAD | B | 600 | −63.057 | 48.329 | 57.770 | 1.00 | 65.73 | B |
| ATOM | 7166 | AC3* | FAD | B | 600 | −60.832 | 48.876 | 58.378 | 1.00 | 64.85 | B |
| ATOM | 7167 | AO3* | FAD | B | 600 | −59.921 | 49.721 | 57.714 | 1.00 | 62.75 | B |
| ATOM | 7168 | AC2* | FAD | B | 600 | −61.849 | 49.583 | 59.243 | 1.00 | 64.98 | B |
| ATOM | 7169 | AO2* | FAD | B | 600 | −61.472 | 50.818 | 59.798 | 1.00 | 62.70 | B |
| ATOM | 7170 | AC1* | FAD | B | 600 | −63.063 | 49.631 | 58.310 | 1.00 | 67.29 | B |
| ATOM | 7171 | AN9 | FAD | B | 600 | −64.353 | 49.868 | 58.969 | 1.00 | 69.52 | B |
| ATOM | 7172 | AC8 | FAD | B | 600 | −64.876 | 49.189 | 60.046 | 1.00 | 70.53 | B |
| ATOM | 7173 | AN7 | FAD | B | 600 | −66.064 | 49.654 | 60.403 | 1.00 | 71.83 | B |
| ATOM | 7174 | AC5 | FAD | B | 600 | −66.323 | 50.680 | 59.521 | 1.00 | 71.71 | B |
| ATOM | 7175 | AC6 | FAD | B | 600 | −67.423 | 51.562 | 59.391 | 1.00 | 72.41 | B |
| ATOM | 7176 | AN6 | FAD | B | 600 | −68.481 | 51.536 | 60.181 | 1.00 | 73.17 | B |
| ATOM | 7177 | AN1 | FAD | B | 600 | −67.372 | 52.502 | 58.375 | 1.00 | 73.01 | B |
| ATOM | 7178 | AC2 | FAD | B | 600 | −66.272 | 52.552 | 57.536 | 1.00 | 72.79 | B |
| ATOM | 7179 | AN3 | FAD | B | 600 | −65.187 | 51.737 | 57.612 | 1.00 | 70.55 | B |
| ATOM | 7180 | AC4 | FAD | B | 600 | −65.261 | 50.820 | 58.619 | 1.00 | 70.68 | B |
| ATOM | 7181 | N1 | FAD | B | 600 | −51.429 | 38.362 | 57.882 | 1.00 | 54.20 | B |
| ATOM | 7182 | C2 | FAD | B | 600 | −50.318 | 37.880 | 57.201 | 1.00 | 56.88 | B |
| ATOM | 7183 | O2 | FAD | B | 600 | −50.417 | 37.352 | 56.098 | 1.00 | 58.60 | B |
| ATOM | 7184 | N3 | FAD | B | 600 | −49.052 | 38.015 | 57.806 | 1.00 | 56.01 | B |
| ATOM | 7185 | C4 | FAD | B | 600 | −48.833 | 38.596 | 59.040 | 1.00 | 55.00 | B |
| ATOM | 7186 | O4 | FAD | B | 600 | −47.676 | 38.685 | 59.509 | 1.00 | 52.53 | B |
| ATOM | 7187 | C4A | FAD | B | 600 | −49.971 | 39.086 | 59.730 | 1.00 | 55.38 | B |
| ATOM | 7188 | N5 | FAD | B | 600 | −49.836 | 39.694 | 60.980 | 1.00 | 55.06 | B |
| ATOM | 7189 | C5A | FAD | B | 600 | −50.937 | 40.197 | 61.655 | 1.00 | 53.09 | B |
| ATOM | 7190 | C6 | FAD | B | 600 | −50.769 | 40.811 | 62.895 | 1.00 | 50.76 | B |
| ATOM | 7191 | C7 | FAD | B | 600 | −51.842 | 41.347 | 63.617 | 1.00 | 50.30 | B |
| ATOM | 7192 | C7M | FAD | B | 600 | −51.587 | 42.001 | 64.955 | 1.00 | 49.27 | B |
| ATOM | 7193 | C8 | FAD | B | 600 | −53.156 | 41.252 | 63.045 | 1.00 | 50.37 | B |
| ATOM | 7194 | C8M | FAD | B | 600 | −54.359 | 41.801 | 63.757 | 1.00 | 52.57 | B |
| ATOM | 7195 | C9 | FAD | B | 600 | −53.349 | 40.630 | 61.786 | 1.00 | 51.47 | B |
| ATOM | 7196 | C9A | FAD | B | 600 | −52.253 | 40.090 | 61.064 | 1.00 | 53.33 | B |
| ATOM | 7197 | N10 | FAD | B | 600 | −52.393 | 39.452 | 59.785 | 1.00 | 54.36 | B |
| ATOM | 7198 | C10 | FAD | B | 600 | −51.262 | 38.952 | 59.113 | 1.00 | 54.50 | B |
| ATOM | 7199 | C1* | FAD | B | 600 | −53.734 | 39.300 | 59.131 | 1.00 | 58.81 | B |
| ATOM | 7200 | C2* | FAD | B | 600 | −54.045 | 40.306 | 58.015 | 1.00 | 61.94 | B |
| ATOM | 7201 | O2* | FAD | B | 600 | −53.302 | 41.477 | 58.240 | 1.00 | 63.28 | B |
| ATOM | 7202 | C3* | FAD | B | 600 | −55.557 | 40.596 | 58.031 | 1.00 | 62.80 | B |
| ATOM | 7203 | O3* | FAD | B | 600 | −56.298 | 39.389 | 57.803 | 1.00 | 63.67 | B |
| ATOM | 7204 | C4* | FAD | B | 600 | −56.070 | 41.575 | 56.955 | 1.00 | 64.14 | B |
| ATOM | 7205 | O4* | FAD | B | 600 | −55.404 | 42.824 | 57.025 | 1.00 | 64.37 | B |
| ATOM | 7206 | C5* | FAD | B | 600 | −57.574 | 41.801 | 57.117 | 1.00 | 66.04 | B |
| ATOM | 7207 | O5* | FAD | B | 600 | −57.976 | 42.711 | 56.088 | 1.00 | 66.91 | B |
| ATOM | 7208 | P | FAD | B | 600 | −59.462 | 43.226 | 55.944 | 1.00 | 69.02 | B |
| ATOM | 7209 | O1P | FAD | B | 600 | −59.437 | 44.139 | 54.774 | 1.00 | 67.78 | B |
| ATOM | 7210 | O2P | FAD | B | 600 | −60.403 | 42.062 | 55.867 | 1.00 | 68.07 | B |
| ATOM | 7211 | O3P | FAD | B | 600 | −59.831 | 43.976 | 57.291 | 1.00 | 67.47 | B |
| ATOM | 7212 | C1 | INH | A | 601 | −39.713 | −6.665 | 28.521 | 1.00 | 57.06 | A |
| ATOM | 7213 | C10 | INH | A | 601 | −39.060 | −7.840 | 28.849 | 1.00 | 56.85 | A |
| ATOM | 7214 | BR1 | INH | A | 601 | −39.728 | −9.247 | 29.869 | 1.00 | 52.87 | A |
| ATOM | 7215 | C9 | INH | A | 601 | −37.775 | −7.709 | 28.260 | 1.00 | 57.72 | A |
| ATOM | 7216 | C12 | INH | A | 601 | −36.604 | −8.700 | 28.332 | 1.00 | 58.10 | A |
| ATOM | 7217 | F4 | INH | A | 601 | −35.600 | −8.174 | 29.067 | 1.00 | 60.01 | A |
| ATOM | 7218 | F3 | INM | A | 601 | −36.968 | −9.880 | 28.904 | 1.00 | 56.89 | A |
| ATOM | 7219 | F2 | INH | A | 601 | −36.165 | −8.911 | 27.075 | 1.00 | 59.72 | A |
| ATOM | 7220 | N8 | INH | A | 601 | −37.684 | −6.511 | 27.562 | 1.00 | 58.36 | A |
| ATOM | 7221 | C11 | INH | A | 601 | −36.525 | −5.914 | 26.928 | 1.00 | 60.05 | A |
| ATOM | 7222 | N2 | INH | A | 601 | −38.837 | −5.843 | 27.762 | 1.00 | 58.43 | A |
| ATOM | 7223 | C2 | INH | A | 601 | −41.082 | −6.190 | 28.866 | 1.00 | 58.10 | A |
| ATOM | 7224 | C3 | INH | A | 601 | −41.187 | −4.888 | 29.402 | 1.00 | 57.33 | A |
| ATOM | 7225 | C4 | INH | A | 601 | −42.433 | −4.344 | 29.752 | 1.00 | 59.22 | A |
| ATOM | 7226 | C5 | INH | A | 601 | −43.615 | −5.131 | 29.559 | 1.00 | 61.60 | A |
| ATOM | 7227 | CL1 | INH | A | 601 | −45.193 | −4.528 | 29.962 | 1.00 | 70.46 | A |
| ATOM | 7228 | C6 | INH | A | 601 | −43.535 | −6.433 | 29.025 | 1.00 | 59.85 | A |
| ATOM | 7229 | C7 | INH | A | 601 | −42.288 | −6.979 | 28.672 | 1.00 | 58.95 | A |
| ATOM | 7230 | F1 | INH | A | 601 | −42.258 | −8.205 | 28.171 | 1.00 | 59.32 | A |
| ATOM | 7231 | C13 | INH | A | 601 | −42.470 | −2.949 | 30.309 | 1.00 | 57.04 | A |
| ATOM | 7232 | O1 | INH | A | 601 | −41.903 | −1.940 | 29.653 | 1.00 | 56.12 | A |
| ATOM | 7233 | O2 | INH | A | 601 | −43.033 | −2.749 | 31.363 | 1.00 | 57.22 | A |
| ATOM | 7234 | C13 | TRI | A | 603 | −38.241 | −.656 | 40.601 | 1.00 | 85.81 | A |
| ATOM | 7235 | C14 | TRI | A | 603 | −39.196 | −1.627 | 40.215 | 1.00 | 85.30 | A |
| ATOM | 7236 | C15 | TRI | A | 603 | −40.548 | −1.294 | 39.924 | 1.00 | 84.87 | A |
| ATOM | 7237 | O1 | TRI | A | 603 | −41.443 | −2.338 | 39.540 | 1.00 | 82.00 | A |
| ATOM | 7238 | C1 | TRI | A | 603 | −42.414 | −2.158 | 38.495 | 1.00 | 79.20 | A |
| ATOM | 7239 | C2 | TRI | A | 603 | −42.305 | −3.263 | 37.429 | 1.00 | 78.44 | A |
| ATOM | 7240 | O2 | TRI | A | 603 | −42.486 | −2.588 | 36.170 | 1.00 | 79.51 | A |
| ATOM | 7241 | C3 | TRI | A | 603 | −41.429 | −2.814 | 35.218 | 1.00 | 78.62 | A |
| ATOM | 7242 | C4 | TRI | A | 603 | −41.286 | −1.516 | 34.389 | 1.00 | 78.92 | A |
| ATOM | 7243 | O3 | TRI | A | 603 | −40.174 | −1.637 | 33.483 | 1.00 | 78.27 | A |
| ATOM | 7244 | C16 | TRI | A | 603 | −40.922 | .077 | 40.034 | 1.00 | 86.26 | A |
| ATOM | 7245 | C17 | TRI | A | 603 | −39.958 | 1.044 | 40.424 | 1.00 | 87.06 | A |
| ATOM | 7246 | C18 | TRI | A | 603 | −38.584 | .723 | 40.723 | 1.00 | 87.73 | A |
| ATOM | 7247 | C5 | TRI | A | 603 | −37.564 | 1.869 | 41.156 | 1.00 | 90.00 | A |
| ATOM | 7248 | C8 | TRI | A | 603 | −38.378 | 3.087 | 41.754 | 1.00 | 90.86 | A |
| ATOM | 7249 | C7 | TRI | A | 603 | −36.636 | 1.271 | 42.268 | 1.00 | 89.96 | A |
| ATOM | 7250 | C6 | TRI | A | 603 | −36.635 | 2.277 | 39.904 | 1.00 | 92.29 | A |
| ATOM | 7251 | C9 | TRI | A | 603 | −36.346 | 3.689 | 39.182 | 1.00 | 93.40 | A |
| ATOM | 7252 | C12 | TRI | A | 603 | −35.463 | 3.553 | 37.898 | 1.00 | 93.25 | A |
| ATOM | 7253 | C11 | TRI | A | 603 | −37.643 | 4.486 | 38.764 | 1.00 | 93.49 | A |
| ATOM | 7254 | C10 | TRI | A | 603 | −35.509 | 4.576 | 40.177 | 1.00 | 93.44 | A |
| ATOM | 7255 | C1 | INH | B | 601 | −46.588 | 35.632 | 65.116 | 1.00 | 62.63 | B |
| ATOM | 7256 | C10 | INH | B | 601 | −47.368 | 35.550 | 66.249 | 1.00 | 61.24 | B |
| ATOM | 7257 | BR1 | INH | B | 601 | −46.926 | 34.630 | 67.801 | 1.00 | 62.38 | B |
| ATOM | 7258 | C9 | INH | B | 601 | −48.550 | 36.274 | 65.934 | 1.00 | 62.42 | B |
| ATOM | 7259 | C12 | INH | B | 601 | −49.827 | 36.475 | 66.814 | 1.00 | 63.37 | B |
| ATOM | 7260 | F4 | INH | B | 601 | −50.820 | 35.720 | 66.297 | 1.00 | 63.39 | B |
| ATOM | 7261 | F3 | INH | B | 601 | −49.655 | 36.090 | 68.101 | 1.00 | 64.90 | B |
| ATOM | 7262 | F2 | INH | B | 601 | −50.202 | 37.768 | 66.810 | 1.00 | 63.34 | B |
| ATOM | 7263 | N8 | INH | B | 601 | −48.432 | 36.822 | 64.658 | 1.00 | 62.11 | B |
| ATOM | 7264 | C11 | INH | B | 601 | −49.423 | 37.517 | 63.876 | 1.00 | 63.73 | B |
| ATOM | 7265 | N2 | INH | B | 601 | −47.268 | 36.387 | 64.131 | 1.00 | 63.22 | B |
| ATOM | 7266 | C2 | INH | B | 601 | −45.256 | 35.018 | 64.854 | 1.00 | 63.69 | B |
| ATOM | 7267 | C3 | INH | B | 601 | −45.095 | 34.306 | 63.638 | 1.00 | 64.77 | B |
| ATOM | 7268 | C4 | INH | B | 601 | −43.874 | 33.675 | 63.308 | 1.00 | 66.18 | B |
| ATOM | 7269 | C5 | INH | B | 601 | −42.794 | 33.768 | 64.238 | 1.00 | 66.88 | B |
| ATOM | 7270 | CL1 | INH | B | 601 | −41.255 | 33.030 | 63.970 | 1.00 | 73.36 | B |
| ATOM | 7271 | C6 | INH | B | 601 | −42.941 | 34.476 | 65.440 | 1.00 | 66.26 | B |
| ATOM | 7272 | C7 | INH | B | 601 | −44.148 | 35.105 | 65.762 | 1.00 | 64.07 | B |
| ATOM | 7273 | F1 | INH | B | 601 | −44.228 | 35.755 | 66.904 | 1.00 | 64.24 | B |
| ATOM | 7274 | C13 | INH | B | 601 | −43.777 | 32.930 | 61.989 | 1.00 | 65.64 | B |
| ATOM | 7275 | O1 | INH | B | 601 | −43.555 | 33.578 | 60.851 | 1.00 | 65.46 | B |
| ATOM | 7276 | O2 | INH | B | 601 | −43.905 | 31.721 | 61.975 | 1.00 | 68.18 | B |
| ATOM | 7277 | C13 | TRI | B | 603 | −48.937 | 22.863 | 60.689 | 1.00 | 86.13 | B |
| ATOM | 7278 | C14 | TRI | B | 603 | −48.016 | 23.282 | 61.691 | 1.00 | 85.45 | B |
| ATOM | 7279 | C15 | TRI | B | 603 | −46.644 | 23.507 | 61.401 | 1.00 | 85.61 | B |
| ATOM | 7280 | O1 | TRI | B | 603 | −45.778 | 23.937 | 62.431 | 1.00 | 83.66 | B |
| ATOM | 7281 | C1 | TRI | B | 603 | −44.783 | 24.954 | 62.157 | 1.00 | 83.29 | B |
| ATOM | 7282 | C2 | TRI | B | 603 | −45.091 | 26.298 | 62.869 | 1.00 | 83.42 | B |
| ATOM | 7283 | O2 | TRI | B | 603 | −44.894 | 27.325 | 61.869 | 1.00 | 84.12 | B |
| ATOM | 7284 | C3 | TRI | B | 603 | −45.509 | 28.585 | 62.183 | 1.00 | 84.00 | B |
| ATOM | 7285 | C4 | TRI | B | 603 | −45.936 | 29.227 | 60.840 | 1.00 | 85.18 | B |
| ATOM | 7286 | O3 | TRI | B | 603 | −47.064 | 30.105 | 61.048 | 1.00 | 84.11 | B |
| ATOM | 7287 | C16 | TRI | B | 603 | −46.211 | 23.293 | 60.067 | 1.00 | 87.58 | B |
| ATOM | 7288 | C17 | TRI | B | 603 | −47.130 | 22.874 | 59.075 | 1.00 | 88.05 | B |
| ATOM | 7289 | C18 | TRI | B | 603 | −48.524 | 22.643 | 59.332 | 1.00 | 87.72 | B |
| ATOM | 7290 | C5 | TRI | B | 603 | −49.489 | 22.183 | 58.146 | 1.00 | 89.11 | B |
| ATOM | 7291 | C8 | TRI | B | 603 | −48.605 | 21.825 | 56.885 | 1.00 | 89.82 | B |
| ATOM | 7292 | C7 | TRI | B | 603 | −50.259 | 20.906 | 58.634 | 1.00 | 89.15 | B |
| ATOM | 7293 | C6 | TRI | B | 603 | −50.558 | 23.353 | 57.833 | 1.00 | 89.70 | B |
| ATOM | 7294 | C9 | TRI | B | 603 | −50.864 | 24.181 | 56.483 | 1.00 | 91.02 | B |
| ATOM | 7295 | C12 | TRI | B | 603 | −49.700 | 25.130 | 56.027 | 1.00 | 90.65 | B |
| ATOM | 7296 | C11 | TRI | B | 603 | −51.263 | 23.264 | 55.258 | 1.00 | 90.82 | B |
| ATOM | 7297 | C10 | TRI | B | 603 | −52.106 | 25.103 | 56.772 | 1.00 | 90.23 | B |
| END | | | | | | | | | | | |

### REFERENCES

1. Arnould S., Takahashi M. & Camadro J. M. (1999): Acylation stabilizes a protease-resistant conformation of protoporphyrinogen oxidase, the molecular target of diphenyl ether-type herbicides. Proc. Natl. Acad. Sci. USA 96, 14825-14830.
2. Binda C. et al. (1999): A 30 angstrom long U-shaped catalytic tunnel in the crystal structure of polyamine oxidase. Structure 7, 265-276.
3. Binda C., Newton-Vinson P., Hubalek F., Edmondson D. E. & Mattevi A. (2002): Structure of human monoamine oxidase B, a drug target for the treatment of neurological disorders. Nature Struct. Biol. 9, 22-26.
4. Blundel, T., Carney, D., Gardner, S., Hayes, F., Howlin, B., Hubbard, T., Overington, J., Singh, D. A., Sibanda, B. L., and Sutcliffe, M. (1988): Knowledge-based protein modelling and design. Eur. J. Biochem. 172, 513-520.
5. Böhm H. J. (1992): LUDI: Rule-Based Automatic Design of New Substituents for Enzyme Inhibitor Leads. J. Comp.-Aided Mol. Design 6, 593-606.
6. Brünger et al. (1998): Recent developments for the efficient crystallographic refinement of macromolecular structures. Current Opinion in Structural Biology 8, 606-611.
7. Brünger A. T., Adams P. D., Clore G. M., DeLano W. L., Gros P., Grosse-Kunstleve R. W., Jiang J.-S., Kuszewski J., Nilges M., Pannu Navraj S., Read R. J., Rice L. M., Simonson T., Warren G. L. (1998): Crystallography & NMR System: a new software suite for macromolecular structure determination. Acta Crystallographica, Section D, 54 905-921.
8. Fortelle E. de la & Bricogne G. (1997). Maximum-Likelihood Heavy-Atom Parameter Refinement for Multiple Isomorphous Replacement and Multiwavelength Anomalous Diffraction Methods. In Methods in Enzymology, Vol. 276. Macromolecular Crystallography Part A. C. W. Carter and R. M. Sweet ed. Academic Press, San Diego, pp. 472-494.
9. Greer, J. (1985): Model structure for the Inflammatory Protein C5a. Science 228, 1055.
10. Jones T. A., Zou J. Y., Cowan S. W. & Kjeldgaard M. (1991): Improved methods for building protein models in electron density maps and the location of errors in these models. Acta Cryst. A 47, 110-119.
11. Jones G. et al. (1997): Development and Validation of a Genetic Algorithm for Flexible Docking. J. Mol. Biol. 267, 727-748.
12. Kabsch W. (1999): Automatic Processing of Rotation Diffraction Data from Crystals of Initially Unknown Symmetry and Cell Constants. J. Appl. Cryst. 32, 120-124.
13. Lermontova I., Kruse E., Mock H. P. & Grimm B. (1997): Cloning and characterization of a plastidal and a mitochondrial isoform of tobacco protoporphyrinogen IX oxidase. Proc. Natl. Acad. Sci. USA 94, 8895-8900.
14. Loll P. J., Picot D. & Garavito R. M. (1995): The Structural Basis of Aspirin Activity Inferred from the Crystal-Structure of Inactivated Prostaglandin H-2 Synthase. Nature Structural Biology 2, 637-643.
15. Mizutani, H. et al. (1996): Three-dimensional structure of porcine kidney D-amino acid oxidase at 3.0 angstrom resolution. J. Biochem. 120, 14-17.
16. Moon J. B. & Howe W. J. (1991): Computer design of bioactive molecules: a method for receptor-based de novo ligand design. Proteins 11, 314-328.
17. Navaza (1994), AMoRe: an Automated Package for Molecular Replacement. Acta Crystallographica, A50, 157-163.
18. Nishibata Y. & Itai A. (1991): Automatic creation of drug candidate structures based on receptor structure. Starting point for artificial lead generation. Tetrahedron 47, 8985-8990.
24. Rarey M. et al. (1996): Predicting Receptor-Ligand Interactions by an Incremental Construction Algorithm. J. Mol. Biol. 261, 470-489.
25. Weeks C. M. & Miller R. (1999): The design and implementation of SnB version 2.0. J. Appl. Crystall. 32, 120-124.

1. A crystal of a protoporphyrinogen IX oxidase.

2. The crystal according to claim 1, comprising space group C222₁.

3. The crystal according to claim 1 comprising cell constants 119.1, 147.3 and 127.0 Å.

4. The crystal according to claim 1 wherein said crystal is the crystal of a Nicotiana tabacum protoporphyrinogen IX oxidase.

5. The crystal according to claim 1 wherein the protoporphyrinogen Ix oxidase is complexed with a low-molecular-weight molecule.

6. The crystal according to claim 1 wherein the protoporphyrinogen IX oxidase is complexed with 4-bromo-3-(5′-carboxy-4′-chloro-2′-fluorophenyl)-1-methyl-5-trifluoromethylpyrazole.

7. The crystal according to claim 1 wherein said crystal has the structural coordinates defined in Table 2.

8. The crystal according to claim 1 comprising a ligand-binding pocket which is defined by the following amino acids at a distance of 4.5 Å from the bound ligand: Arg⁹⁸, Ala¹⁷⁴, Gly¹⁷⁵, Thr¹⁷⁶, Cys¹⁷⁷, Gly¹⁷⁸, Leu³³⁴, Phe³⁵³, Gly³⁵⁴, Val³⁵⁵, leu³⁵⁶, Leu³⁶⁹, Gly³⁷⁰, Thr³⁷¹, Leu³⁷², Phe³⁹², Phe⁴³⁹.

9. The crystal having essentially the same structure as defined in claim 7, wherein at least one amino acid according to SEQ ID NO:1 has been replaced with another amino acid and/or one amino acid has been deleted and/or one amino acid has been inserted.

10. A process for crystallizing a selenomethionine derivative of a PPO, comprising the following steps:
(a) preparing a recombinant preparation of a PPO in a host cell in the presence of selenomethionine,
(b) isolating the PPO from the host cell
(c) purifying the PPO, and
(d) preparing a crystal according claim 1 from said purified PPO.

11. The process according to claim 10, wherein the crystal is generated by means of sitting-drop vapour diffusion.

12. The process according to claim 10 wherein the crystallization solution comprises 10% polyethylene glycol 1000, 0.1 M sodium citrate/HCl, pH 4.0 and 0.2 M sodium chloride.

13. A process for determining the structure of a PPO which has an identity of at least 40% to the sequence according to SEQ ID NO: 1, comprising the following steps:
(a) aligning the amino acid sequence of the PPO with the amino acid sequence according to SEQ ID NO: 1,
(b) modelling the structure of the corresponding homologous regions of the PPO according to the structural data for these regions of Table 2, and
(c) determining the conformation of the PPO.

14. A process for identifying one or more ligands of a protoporphyrinogen IX oxidase, comprising the following steps:
(a) producing a three-dimensional structure of a PPO,
(b) producing the three-dimensional structure of a candidate molecule, and
(c) fitting the three-dimensional structure of the candidate molecule into the three-dimensional structure of the PPO, and
(d) selecting the candidate molecule which is capable of interacting specifically with the PPO.

15. A process for identifying one or more ligands of a protoporphyrinogen IX oxidase, comprising the following steps:
(a) producing a three-dimensional structure of a PPO,
(b) modelling a compound capable of interacting specifically with the PPO.

16. The process according to claim 14 wherein in step (a) only the coordinates of at least two atoms of a PPO are produced.

17. The process according to claim 14 wherein in step (a) the coordinates of atoms selected from the amino acids Arg⁹⁸, Ala¹⁷⁴, Gly¹⁷⁵, Thr¹⁷⁶, Cys¹⁷⁷, Gly¹⁷⁸, Leu³³⁴, Phe ³⁵³, Gly⁵⁴, Val³⁵⁵, Leu³⁵⁶, Leu³⁶⁹, Gly³⁷⁰, Thr³⁷¹, Leu³⁷², Phe³⁹², Phe ⁴³⁹ are produced.

18. The process according to claim 14 wherein the coordinates in step (a) correspond to the structural coordinates according to Table 2.

19. The process according to claim 14 additionally comprising the following steps:
(a) synthesizing the identified ligand,
(b) forming a complex of a PPO and the ligand,
(c) X-ray structural analysis of the complex, and
(d) determining the ability of the ligand to interact specifically with the PPO.

20. The process according to claim 14 additionally comprising the following steps:
(a) synthesizing the identified ligand, and
(b) determining the ability of the ligand to inhibit the enzymic activity of the PPO in vitro.

21. A process for determining the structure of a ligand bound to a PPO, comprising the following steps:
(a) preparing a crystal of the complex of the PPO and the ligand, and
(b) determining the structure of the complex by using the structural coordinates according to Table 2.

22. A process for controlling undesired plants comprising allowing one or more compounds found by the process according to claim 14 to act on undesired plants and/or their habitat.

23. A method for identifying a herbicide comprising the step of identifying a herbicide using the crystal of a protoporphyrinogen IX oxidase according to claim 1.

24. The process according to claim 15 wherein in step (a) only the coordinates of at least two atoms of a PPO are produced.

25. The process according to claim 15 wherein in step (a) the coordinates of atoms selected from the amino acids Arg⁹⁸, Ala¹⁷⁴, Gly¹⁷⁵, Thr¹⁷⁶, Cys¹⁷⁷, Gly¹⁷⁸, Leu³³⁴, Phe³, Gly¹⁵⁴, Val¹⁵⁵, Leu³⁵⁶, Leu³⁶⁹, Gly³⁷⁰, Thr³⁷¹, Leu³⁷², Phe³⁹², Phe ⁴³⁹ are produced.

26. The process according to claim 15 wherein the coordinates in step (a) correspond to the structural coordinates according to Table 2.

27. The process according to claim 15 additionally comprising the following steps:
(a) synthesizing the identified ligand,
(b) forming a complex of a PPO and the ligand,
(c) X-ray structural analysis of the complex, and
(d) determining the ability of the ligand to interact specifically with the PPO.

28. The process according to claim 15 additionally comprising the following steps:
(a) synthesizing the identified ligand, and
(c) determining the ability of the ligand to inhibit the enzymatic activity of the PPO in vitro.

29. A process for controlling undesired plants comprising allowing one or more compounds found by the process according to claim 15 to act on undesired plants and/or their habitat.


SEQUENCE LISTING (1 sequence)

SEQ ID NO: 1 - protein, 503 aa, Nicotiana tabacum
  PRT (1)..(503)
APSAGEDKHSSAKRVAVIGAGVSGLAAAYKLKIHGLNVTVFEAEGKAGGKLRSVSQDGLIWDEGANTMTESEGDVTFLIDSLGLREKQQFPLSQNKRYIARNGTPVLLPSNPIDLIKSNFLSTGSKLQMLLEPILWKNKKLSQVSDSHESVSGFFQRHFGKEVVDYLIDPFVAGTCGGDPDSLSMHHSFPELWNLEKRFGSVILGAIRSKLSPKNEKKQGPPQTSANKKRQRGSFSFLGGMQTLTDAICKDLREDELRLNSRVLELSCSCTEDSAIDSWSIISASPHKRQSEEESFDAVIMTAPLCDVKSMKIAKRGNPFLLNFIPEVDYVPLSVVITTFKRENVKYPLEGFGVLVPSKEQQHGLKTLGTLFSSMMFPDRAPNNVYLYTTFVGGSRNRELAKASRTELKEIVTSDLKQLLGAEGEPTYVNHLYWSKAFPLYGHNYDSVLDAIDKMEKNLPGLFYAGNHRGGLSVGKALSSGCNAADLVISYLESVSTDSKRHC